(19) United States
(12) Patent Application Publication
(10) Pub. No.: US 2026/0226068 A1
(43) Pub. Date: Aug. 6, 2026

(54) IMIDAZOTRIAZINE IL-17A MODULATORS AND USES THEREOF

(71) Applicant: DICE ALPHA, INC., Indianapolis, IN (US)

(72) Inventors: Hassane BELABED, San Francisco, CA (US); Gary Edward Lee BRANDT, Alameda, CA (US); Paul R. FATHEREE, San Francisco, CA (US); Michael David FREIDBERG, Belmont, CA (US); Maureen Kay REILLY, Emerald Hills, CA (US)

(21) Appl. No.: 19/156,336

(22) PCT Filed: Feb. 9, 2024
(86) PCT No.: PCT/US2024/015156

§ 371 Date: Aug. 13, 2025

Related U.S. Application Data
(60) Provisional application No. 63/484,684, filed on Feb. 13, 2023.
(60) Provisional application No. 63/511,519, filed on Jun. 30, 2023.
(60) Provisional application No. 63/516,399, filed on Jul. 28, 2023.
(60) Provisional application No. 63/602,304, filed on Nov. 22, 2023.

(51) Int. Cl. C07D 487/04; A61K 31/53; A61K 31/5377; A61K 31/5386; A61K 31/55; A61P 29/00; C07D 519/00

(52) U.S. Cl. CPC C07D 487/04; A61K 31/53; A61K 31/5377; A61K 31/5386; A61K 31/55; A61P 29/00; C07D 519/00

(57) ABSTRACT

The disclosure herein provides lactam substituted imidazotriazine compounds or salts of Formula (I′), (I), (II), (II-a), (II-b), or (A), and pharmaceutical compositions thereof, for the modulation of IL-17A. These compounds are useful in the treatment of inflammatory conditions such as psoriasis.

### BACKGROUND OF THE INVENTION

The IL-17 family consists of six cytokines (IL-17A through IL-17F). Interleukin-17A (IL-17A), is an established pro-inflammatory cytokine, which is involved in the induction of IL-6, IL-8, G-CSF, TNF-α, IL-1β, PGE2, and IFN-γ, as well as numerous chemokines and other effectors. IL-17A can form homodimers or heterodimers with its family member, IL-17F and can bind to both IL-17 receptors, IL-17 RA and IL-17 RC, in order to mediate signaling. IL-17A is a major pathological cytokine expressed by Th17 cells, which are involved in the pathology of inflammation and autoimmunity, and also CD8+ T cells, γδ cells, NK cells, NKT cells, macrophages and dendritic cells. Additionally, IL-17A and Th17 are necessary for defense against various microbes despite their involvement in inflammation and autoimmune disorders. Further, IL-17A can act in cooperation with other inflammatory cytokines such as TNF-α, IFN-γ, and IL-1β to mediate pro-inflammatory effects.

To date, there are a few biologics (Secukinumab and Ixekizumab) that have been approved to modulate IL-17A for the treatment of inflammatory diseases, such as psoriasis, ankylosing spondylitis, and psoriatic arthritis. These treatments require injection to a patient as they are not readily absorbed by the gut when orally ingested. Further, these approved biologic treatments have a high cost of entry for patients, limiting the availability to the patient population in need thereof.

There are a few small molecule modulators of IL-17A that have been approved for oral administration. However, while these have the convenience of oral administration and a lower cost of entry for patients, they lack the efficacy of approved biologics. Therefore, there exists a need for the development of potent small molecule IL-17A modulators for the treatment of inflammatory diseases and other associated disorders.

### SUMMARY OF THE INVENTION

In some aspects, the present disclosure provides a compound represented by the structure of Formula (I′):

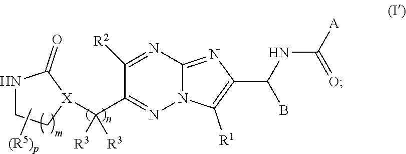

or a pharmaceutically acceptable salt thereof wherein:
X is selected from CR⁴ or N;
A is selected from (a) and (b):
   (a) C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN;
   (b) —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
      C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), —CN; and C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN; and
      C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
each R′ is selected from hydrogen, halogen, —OR¹⁸A, —N(R^{18A})₂, —C(O)R^{18A}, —C(O)N(R^{18A})₂, —NO₂, —CN; and C₁₋₆ alkyl optionally substituted with one or more from halogen, —OR^{18A}, —N(R^{18A})₂, —C(O)R^{18A}, —C(O)N(R^{18A})₂, —NO₂, and —CN;
each R″ is selected from hydrogen, halogen, —OR^{19A}, —N(R^{19A})₂, —C(O)R^{19A}, —C(O)N(R^{19A})₂, —NO₂, —CN; and C₁₋₆ alkyl optionally substituted with one or more from halogen, —OR^{19A}, —N(R^{19A})₂, —C(O)R^{19A}, —C(O)N(R^{19A})₂, —NO₂, and —CN;
B is selected from —C(R⁶)₃ and C₃₋₁₀ carbocycle, wherein the C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) hydrogen, halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R² is selected from:
   hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN;
   C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN; and C₁₋₆ alkyl optionally substituted with one or more substituents independently selected from halogen, —OR^{15A}, —SR^{1A5}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN;
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, 3- to 10-membered heterocycle, and C₁₋₆ alkyl, the C₃₋₁₀ carbocycle, 3- to 10-membered heterocycle, and C₁₋₆ alkyl, are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl);
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl and C₃₋₁₀ carbocycle, each of which is optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
or two R⁵ are taken together with the atoms to which they are connected, come together to form a C₃₋₆ carbocycle or 3- to 6-membered heterocycle; the C₃₋₆ carbocycle and 3- to 6-membered heterocycle are each optionally substituted with one or more substituents selected from: halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), —CN, C₁₋₁₀ alkyl and C₃₋₁₀ carbocycle, the C₁₋₁₀ alkyl and C₃₋₁₀ carbocycle are each optionally substituted with one or more substituents independently selected from halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN;
or R⁴ and one R⁵ may come together to form a 3- to 6-membered carbocycle, wherein the 3- to 6-membered carbocycle is optionally substituted with one or more substituents independently selected from:
   halogen, —OR^{20A}, —N(R^{20A})₂, —C(O)R^{20A}, —C(O)N(R^{20A})₂, —NO₂, ═O, ═S, ═N(R^{20A}), —CN;
   C₁₋₁₀ alkyl and C₃₋₁₀ carbocycle, each of which is optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), —CN;
R¹¹, R^{11A}, R¹², R¹³, R¹⁴, R¹⁵, R^{15A}, R¹⁶, R¹⁷, R^{17A}, R^{18A}, R^{19A}, and R^{20A} are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2;
p is selected 0, 1, 2, 3 4, 5, and 6; and
z is selected from 0, 1, and 2.

In certain aspects, the present disclosure provides a compound represented by the structure of Formula (I):

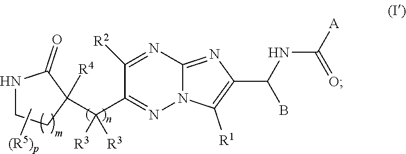

or a pharmaceutically acceptable salt thereof wherein:
A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
each R′ is selected from hydrogen, halogen, —OR^{18A}, —N(R^{18A})₂, —C(O)R^{18A}, —C(O)N(R^{18A})₂, —NO₂, —CN; and C₁₋₆ alkyl optionally substituted with one or more from halogen, —OR^{18A}, —N(R^{18A})₂, —C(O)R^{18A}, —C(O)N(R^{18A})₂, —NO₂, and —CN;
each R″ is selected from hydrogen, halogen, —OR^{19A}, —N(R^{19A})₂, —C(O)R^{19A}, —C(O)N(R^{19A})₂, —NO₂, —CN; and C₁₋₆ alkyl optionally substituted with one or more from halogen, —OR^{19A}, —N(R^{19A})₂, —C(O)R^{19A}, —C(O)N(R^{19A})₂, —NO₂, and —CN;
B is selected from —CH(R⁶)₂ and C₃₋₁₀ carbocycle, wherein the C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN:
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R² is selected from:
   hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN;
   C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN; and
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
R¹¹, R¹², R¹³, R¹⁴, R¹⁵, R^{15A}, R¹⁶, R¹⁷, R^{18A}, and R^{19A} are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2;
p is selected 1, 2, 3 and 4; and
z is selected from 0, 1, and 2.

In certain embodiments, Formula (I′), Formula (I), or Formula (II), is represented by the structure of Formula (II-a):

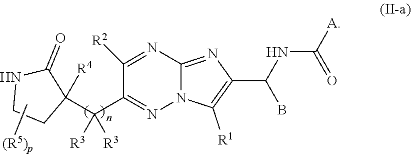

In certain embodiments, Formula (I′), Formula (I), or Formula (II), is represented by the structure of Formula (II-b):

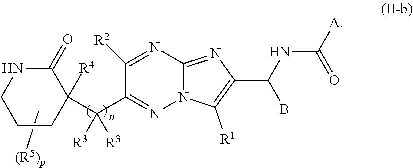

In certain aspects, the present disclosure provides a pharmaceutical composition comprising a compound or salt of Formula (I′), (I), (II), (II-a), or (II-b), and a pharmaceutically acceptable excipient.

In certain aspects, the present disclosure provides a method of modulating IL-17A in a subject in need thereof, comprising administering to the subject a compound or salt of Formula (I′), (I), (II), (II-a), or (II-b) or a pharmaceutical composition thereof.

In certain aspects, the present disclosure provides a method of treating an inflammatory disease or condition comprising administering to a subject in need thereof a compound or salt of Formula (I′), (I), (II), (II-a), or (II-b), or a pharmaceutical composition thereof. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene—refers to a substituted or unsubstituted alkylene chain with from x to y carbons in the alkylene chain. For example, —C₁₋₆ alkylene—may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene—refers to a substituted or unsubstituted alkenylene chain with from x to y carbons in the alkenylene chain. For example, —C₂₋₆ alkenylene—may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene—refers to a substituted or unsubstituted alkynylene chain with from x to y carbons in the alkynylene chain. For example, —C₂₋₆ alkynylene—may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms—any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 5- to 12-membered aromatic ring radical whose ring structure comprise at least one heteroatom, preferably between one to four heteroatoms. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted.

Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro, 2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R′ is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In some aspects, the present disclosure provides a compound represented by the structure of Formula (A):

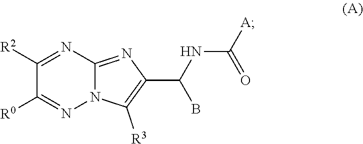

or a pharmaceutically acceptable salt thereof wherein:
A is selected from:
   —O—(C₁₋₁₀ alkyl) and C₁₋₁₀ alkyl, the C₁₋₁₀ alkyl of each is optionally substituted with one or more substituents independently selected from:
      halogen, —OR¹¹, —SR¹¹, —N(R¹¹)₂, —C(O)R¹¹, —C(O)N(R¹¹)₂, —C(O)OR¹¹, —OC(O)R¹¹, —N(R¹¹)C(O)R¹¹, —N(R¹)S(O)₂R¹¹, —C(O)OR¹¹, —OC(O)R¹¹, —S(O)R¹¹, —S(O)₂R¹¹, —NO₂, —CN; and
      3- to 10-membered heterocycle and C₃₋₁₀ carbocycle, any of which is optionally substituted with one or more substituents independently selected from halogen, —OR¹¹, —SR¹¹, —N(R¹¹)₂, —C(O)R¹¹, —C(O)N(R)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   3- to 10-membered heterocycle and C₃₋₁₀ carbocycle, any of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
      C₁₋₁₀ alkyl optionally substituted with one or more substituents independently selected from halogen, —OR¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
      C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —CH(R⁶)₂ and C₃₋₁₀ carbocycle, wherein the C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R⁰ and R² are each independently selected from (a) and (b):

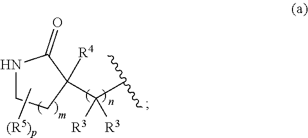

and
   (b) a substituent selected from:
      hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN;
      C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN; and
      C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
   wherein one of R⁰ and R² is selected from (a);
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
R¹¹, R¹², R¹³, R¹⁴, R¹⁵, R¹⁶, and R¹⁷ are each independently selected at each occurrence from: hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2; and
p is selected 1, 2, 3 and 4.

In some aspects, the present disclosure provides a compound represented by the structure of Formula (I′):

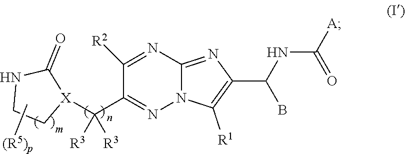

or a pharmaceutically acceptable salt thereof wherein:
X is selected from CR⁴ or N;
A is selected from (a) and (b):
   (a) C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN;
   (b) —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
      C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), —CN; and C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN; and
      C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
each R′ is selected from hydrogen, halogen, —OR^{18A}, —N(R^{18A})₂, —C(O)R^{18A}, —C(O)N(R^{18A})₂, —NO₂, —CN; and C₁₋₆ alkyl optionally substituted with one or more from halogen, —OR^{18A}, —N(R^{18A})₂, —C(O)R^{18A}, —C(O)N(R^{18A})₂, —NO₂, and —CN;
each R″ is selected from hydrogen, halogen, —OR^{19A}, —N(R^{19A})₂, —C(O)R^{19A}, —C(O)N(R^{19A})₂, —NO₂, —CN; and C₁₋₆ alkyl optionally substituted with one or more from halogen, —OR^{19A}, —N(R^{19A})₂, —C(O)R^{19A}, —C(O)N(R^{19A})₂, —NO₂, and —CN;
B is selected from —C(R⁶)₃ and C₃₋₁₀ carbocycle, wherein the C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) hydrogen, halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN:
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R² is selected from:
   hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN;
   C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN; and C₁₋₆ alkyl optionally substituted with one or more substituents independently selected from halogen, —OR^{15A}, —SR^{1A5}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN;
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, 3- to 10-membered heterocycle, and C₁₋₆ alkyl, the C₃₋₁₀ carbocycle, 3- to 10-membered heterocycle, and C₁₋₆ alkyl, are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl);
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl and C₃₋₁₀ carbocycle, each of which is optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
or two R⁵ are taken together with the atoms to which they are connected, come together to form a C₃₋₆ carbocycle or 3- to 6-membered heterocycle; the C₃₋₆ carbocycle and 3- to 6-membered heterocycle are each optionally substituted with one or more substituents selected from: halogen, —OR^{17A}, —SR^{17A}, —N(R^{17A})₂, —C(O)R^{17A}, —C(O)N(R^{17A})₂, —N(R^{17A})C(O)R¹⁷, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), —CN, C₁₋₁₀ alkyl and C₃₋₁₀ carbocycle, the C₁₋₁₀ alkyl and C₃₋₁₀ carbocycle are each optionally substituted with one or more substituents independently selected from halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN;
or R⁴ and one R⁵ may come together to form a 3- to 6-membered carbocycle, wherein the 3- to 6-membered carbocycle is optionally substituted with one or more substituents independently selected from:
   halogen, —OR^{20A}, —N(R^{20A})₂, —C(O)R^{20A}, —C(O)N(R^{20A})₂, —NO₂, ═O, ═S, ═N(R^{20A}), —CN;
   C₁₋₁₀ alkyl and C₃₋₁₀ carbocycle, each of which is optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), and —CN;
R¹¹, R^{11A}, R¹², R¹³, R¹⁴, R¹⁵, R^{15A}, R¹⁶, R¹⁷, R^{17A}, R^{18A}, R^{19A}, and R^{20A} are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2;
p is selected 0, 1, 2, 3 4, 5, and 6; and
z is selected from 0, 1, and 2.

In some embodiments, for the compound or salt of Formula (I′), A is for the compound or salt of Formula (I′), C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN. In some embodiments, A is C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —NO₂, ═O, ═S, ═N(R^{11A}), and —CN.

In some In some embodiments, for the compound or salt of Formula (I′), A is C₁₋₆ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)R^{11A}, —OC(O)R^{11A}, —S(O)R^{11A}, —S(O)₂R^{11A}, —NO₂, ═O, ═S, ═N(R^{11A}), and —CN. In some embodiments, A is C₁₋₆ alkyl optionally substituted with one or more substituents independently selected from halogen, —OR^{11A}, —SR^{11A}, —N(R^{11A})₂, —C(O)R^{11A}, —C(O)N(R^{11A})₂, —NO₂, ═O, ═S, ═N(R^{11A}), and —CN. In some embodiments, A is C₁₋₃ alkyl optionally substituted with one or more substituents independently selected from halogen, —OR^{11A}, —N(R^{11A})₂, —NO₂, ═O, ═S, ═N(R^{11A}), and —CN.

In some embodiments, for the compound or salt of Formula (I′), A is selected —O—(CR′R″)_{z}(3- to 10-membered heterocycle) and —O—(CR′R″)_{z}(C₃₋₁₀ carbocycle), the 3 to 10-membered heterocycle and C₃₋₁₀ carbocycle are each optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), —CN; and C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN; and
   C₃₋₁₀ carbocycle and 3- to 10-membered heterocycle, each of which optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I′), A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from: C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), —CN, and C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN.

In some embodiments, for the compound or salt of Formula (I′), A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3- to 10-membered heterocycle and C₃₋₁₀ carbocycle are each optionally substituted with one or more substituents independently selected from: C₁₋₁₀ alkyl optionally substituted with one or more substituents independently selected C₃₋₁₀ carbocycle and 3- to 10-membered heterocycle, each of which is optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), —CN, and C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN.

In some embodiments, for the compound or salt of Formula (I′), A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from: C₁₋₁₀ alkyl optionally substituted with one or more substituents independently selected C₃₋₁₀ carbocycle and 3- to 10-membered heterocycle, each of which is optionally substituted with one or more C₁₋₁₀ alkyl optionally substituted with one or more substituents independently selected from halogen, —OR^{11A}, —SR^{11A}, —N(R^{11A})₂, —C(O)R^{11A}, —C(O)N(R^{11A})₂, —N(R^{11A})C(O)R^{11A}, —C(O)OR^{11A}, —OC(O)R^{11A}, —S(O)R^{11A}, —S(O)₂R^{11A}, —NO₂, ═O, ═S, ═N(R^{11A}), and —CN. In some embodiments, A is

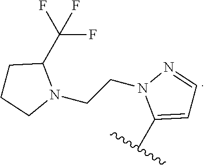 [SMILES: *c1ccnn1CCN1CCCC1C(F)(F)F]

In some embodiments, for the compound or salt of Formula (I′), A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), and —CN. In some embodiments, A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from C₃₋₁₀ carbocycle and 3- to 10-membered heterocycle, each of which is optionally substituted with one or more substituents independently selected from C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from C₃₋₁₀ carbocycle and 3- to 10-membered heterocycle, each of which is optionally substituted with one or more substituents independently selected from C₁₋₆ alkyl optionally substituted with one or more substituents independently selected from: halogen, —OR¹¹, —N(R¹¹)₂, —C(O)R¹¹, —NO₂, ═O, ═N(R¹¹), and —CN.

In some embodiments, for the compound or salt of Formula (I′), R² is selected from:
hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, N(R¹⁵)S(O)₂R¹⁵, —C(O)OR¹⁵, —OC(O)R¹⁵, —S(O)R¹⁵, —S(O)₂R¹⁵, —NO₂, —CN;
C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN; and C₁₋₆ alkyl optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN;
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, 3- to 10-membered heterocycle, and C₁₋₆ alkyl, the C₃₋₁₀ carbocycle, 3- to 10-membered heterocycle, and C₁₋₆ alkyl, are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl).

In some embodiments, for the compound or salt of Formula (I′), R² is selected from C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN, and C₁₋₆ alkyl optionally substituted with one or more substituents independently selected from halogen, —OR^{15A}, —SR^{1A5}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN.

In some embodiments, for the compound or salt of Formula (I′), R² is C₁₋₆ alkyl optionally substituted with one or more substituents independently selected from C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN, and C₁₋₆ alkyl optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN.

In some embodiments, for the compound or salt of Formula (I′), R² is C₁₋₆ alkyl optionally substituted with one or more substituents independently selected from azetidinyl, pyrrolidinyl, piperazinyl, and morpholinyl, the azetidinyl, pyrrolidinyl, piperazinyl, morpholinyl are each optionally substituted with one or more substituents independently selected from halogen, —OR^{15A}, —N(R^{15A})₂, —NO₂, ═O, and —CN, and C₁₋₆ alkyl optionally substituted with one or more substituents independently selected from halogen, —OR^{15A}, —N(R^{15A})₂, —NO₂, and —CN. In some embodiments, R² is selected from

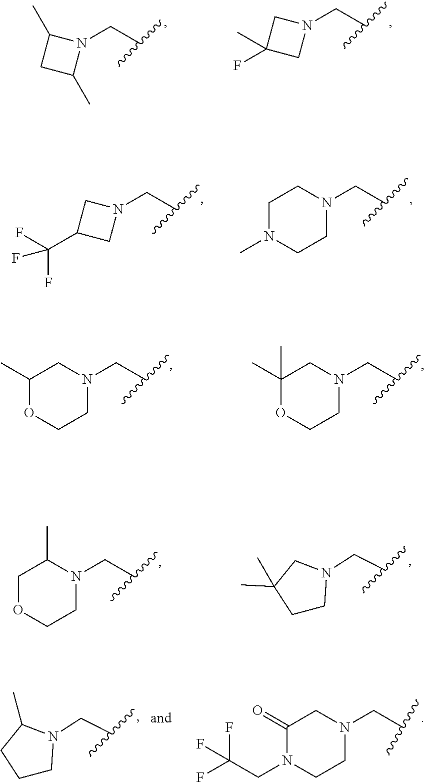

In some embodiments, for the compound or salt of Formula (I′), R² is C₁₋₆ alkyl optionally substituted with one or more 5- to 10-membered bicyclic heterocycle optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), —CN, and C₁₋₆ alkyl optionally substituted with one or more substituents independently selected from halogen, —OR^{15A}, —SR^{1A5}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more 5- to 10-membered bridged heterocycle optionally substituted with one or more substituents independently selected from halogen, —OR^{15A}, —N(R^{15A})₂, —NO₂, and —CN; and C₁₋₆ alkyl optionally substituted with one or more substituents independently selected from halogen, —OR^{15A}, —N(R^{15A})₂, —NO₂, and —CN. In some embodiments, R² is

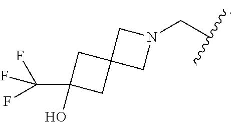 [SMILES: *CN1CC2(C1)CC(O)(C(F)(F)F)C2]

In some embodiments, for the compound or salt of Formula (I′), R² is selected from: C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, 3- to 10-membered heterocycle, and C₁₋₆ alkyl, the C₃₋₁₀ carbocycle, 3- to 10-membered heterocycle and C₁₋₆ alkyl, are each optionally substituted with one or more substituents independently selected from halogen, —OR¹⁵, —SR¹⁵, —N(R¹⁵)₂, —C(O)R⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl).

In some embodiments, for the compound or salt of Formula (I′), R² is selected from: C₃₋₁₀ carbocycle and 3- to 10-membered heterocycle, each of which is optionally substituted with one or more substituents independently selected from C₃₋₁₀ carbocycle, 3- to 10-membered heterocycle, and C₁₋₆ alkyl, the C₃₋₁₀ carbocycle, 3- to 10-membered heterocycle and C₁₋₆ alkyl, are each optionally substituted with one or more substituents independently selected from halogen, —OR¹⁵, —SR^{1S}, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl). In some embodiments, R² is selected from: C₃₋₁₀ carbocycle and 3- to 10-membered heterocycle, each of which is optionally substituted with one or more substituents independently selected from C₃₋₁₀ carbocycle, 3- to 10-membered heterocycle, and C₁₋₆ alkyl, the C₃₋₁₀ carbocycle, 3- to 10-membered heterocycle and C₁₋₆ alkyl, are each optionally substituted with one or more substituents independently selected from C₃₋₁₀ carbocycle, C₃₋₁₀ carbocycle-(C₁₋₆ alkyl), C₃₋₁₀ carbocycle-(C₁₋₆ haloalkyl), 3- to 10-membered heterocycle, 3- to 10-membered heterocycle(C₁₋₆ alkyl), and 3- to 10-membered heterocycle(C₁₋₆ haloalkyl). In some embodiments, R² is selected from:

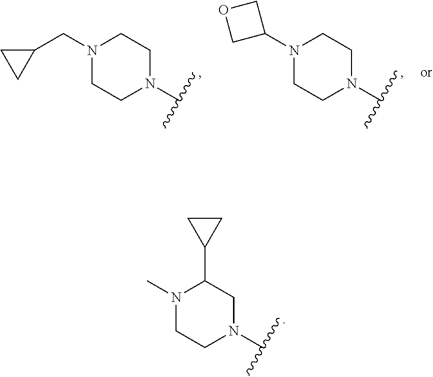 [SMILES: *N1CCN(CC2CC2)CC1 ; *N1CCN(C2COC2)CC1 ; *N1CCN(C)C(C2CC2)C1]

In some embodiments, for the compound or salt of Formula (I′), each R⁵ is independently selected at each occurrence from C₁₋₁₀ alkyl and C₃₋₁₀ carbocycle, each of which is optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁵ is C₃₋₁₀ carbocycle, each of which is optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁵ is C₃₋₆ carbocycle, each of which is optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₆ carbocycle and 3- to 6-membered heterocycle; wherein the C₃₋₆ carbocycle and 3- to 6-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN.

In some embodiments, for the compound or salt of Formula (I′), two R⁵ are taken together with the atoms to which they are connected, come together to form a C₃₋₅ carbocycle or a 3- to 5-membered heterocycle; the C₃₋₆ carbocycle and 3- to 6-membered heterocycle are each optionally substituted with one or more substituents selected from: halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{7A}, —S(O)₂R^{17A}, —NO₂, ═O, ═S, ═N(R^{7A}), and —CN.

In some embodiments, for the compound or salt of Formula (I′), two R⁵ substituents may come together to form a 3- to 6-membered carbocycle, wherein the 3- to 6-membered carbocycle is optionally substituted with one or more substituents independently selected from: —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN.

In some embodiments, for the compound or salt of Formula (I′), two R⁵ substituents may come together to form a C₃₋₆ carbocycle or 3- to 6-membered heterocycle; the C₃₋₆ carbocycle and 3- to 6-membered heterocycle are each optionally substituted with one or more substituents selected from C₁₋₁₀ alkyl and C₃₋₁₀ carbocycle, the C₁₋₁₀ alkyl and C₃₋₁₀ carbocycle are each optionally substituted with one or more substituents independently selected from halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN. In some embodiments, two R⁵ substituents may come together to form a C₃₋₆ carbocycle or 3- to 6-membered heterocycle; the C₃₋₆ carbocycle and 3- to 6-membered heterocycle are each optionally substituted with one or more substituents selected from C₁₋₆ alkyl and C₃₋₆ carbocycle, the C₁₋₁₀ alkyl and C₃₋₁₀ carbocycle are each optionally substituted with one or more substituents independently selected from halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN. In some embodiments, two R⁵ substituents may come together to form a C₃₋₆ carbocycle or 3- to 6-membered heterocycle; the C₃₋₆ carbocycle and 3- to 6-membered heterocycle are each optionally substituted with one or more substituents selected from C₁₋₆ alkyl optionally substituted with one or more substituents independently selected from halogen, —OR^{17A}, —SR^{17A}, —N(R^{17A})₂, —C(O)R^{17A}, —NO₂, ═O, ═S, ═N(R^{17A}), and —CN. In some embodiments, two R⁵ substituents may come together to form a C₃₋₆ carbocycle or 3- to 6-membered heterocycle; the C₃₋₆ carbocycle and 3- to 6-membered heterocycle are each optionally substituted with one or more substituents selected from C₃₋₆ carbocycle optionally substituted with one or more substituents independently selected from halogen, —OR^{17A}, —SR^{17A}, —N(R^{17A})₂, —C(O)R^{17A}, —NO₂, ═O, ═S, ═N(R^{17A}), and —CN.

In some embodiments, for the compound or salt of Formula (I′), two R⁵ substituents on adjacent carbon atoms may come together to form an optionally substituted 3-membered cycloalkyl. In some embodiments, two R⁵ substituents on adjacent carbon atoms may come together to form an optionally substituted 3-membered cycloalkyl, including the two carbon atoms to which the two R⁵ substituents are attached. For example,

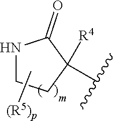

may be represented by, but not limited to,

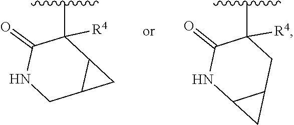

and may be optionally substituted as described herein. In some embodiments, two R⁵ substituents may come together to form a 4-membered cycloalkyl optionally substituted with one or more substituents independently selected from: halogen, —OR^{17A}, —SR^{17A}, —N(R^{17A})₂, —C(O)R^{17A}, —C(O)N(R^{17A})₂, —N(R^{17A})C(O)R^{17A}, —N(R^{17A})S(O)₂R^{17A}, —C(O)OR^{17A}, —OC(O)R^{17A}, —S(O)R^{17A}, —S(O)₂R^{17A}, —NO₂, ═O, ═S, ═N(R^{17A}), and —CN. In some embodiments, two R⁵ substituents on different carbon atoms may come together to form an optionally substituted 4-membered cycloalkyl. In some embodiments, two R⁵ substituents on different carbon atoms may come together to form an optionally substituted 4-membered cycloalkyl, including the two carbon atoms to which the two R⁵ substituents are attached. For example,

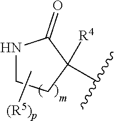

may be represented by, but not limited to

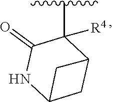

and may be optionally substituted as described herein.

In some embodiments, for the compound or salt of Formula (I′),

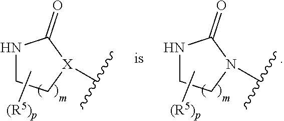

In some embodiments,

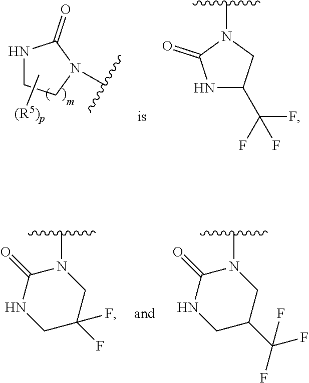

In some embodiments,

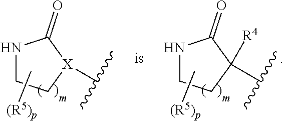

In some embodiments, wherein

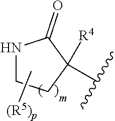

is selected from

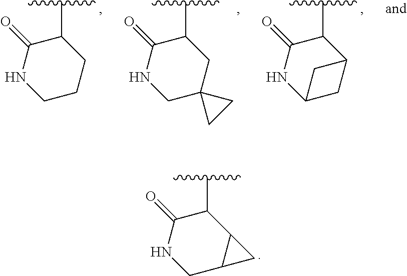 [SMILES: *C1CCCNC1=O ; *C1CC2(CC2)CNC1=O ; *C1C(=O)NC2CC1C2 ; *C1C(=O)NCC2CC21]

In some embodiments, for the compound or salt of Formula (I′), one R⁴ and one R⁵ may come together to form a 3- to 6-membered carbocycle, wherein the 3- to 6-membered carbocycle is optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), —CN, C₁₋₁₀ alkyl and C₃₋₁₀ carbocycle, the C₁₋₁₀ alkyl and C₃₋₁₀ carbocycle are each optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), and —CN. In some embodiments, one R⁴ and one R⁵ may come together to form a 3- to 6-membered carbocycle, wherein the 3- to 6-membered carbocycle is optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), —CN, C₁₋₆ alkyl and C₃₋₆ carbocycle, the C₁₋₆ alkyl and C₃₋₆ carbocycle are each optionally substituted with one or more substituents independently selected from halogen, —OR^{20A}, —N(R^{20A})₂, —C(O)R^{20A}, —C(O)N(R^{20A})₂, —NO₂, ═O, ═S, ═N(R^{20A}), and —CN.

In some embodiments, for the compound or salt of Formula (I′), p is 0.

In some embodiments, for the compound or salt of Formula (I′), p is 5.

In some embodiments, for the compound or salt of Formula (I′), p is 6.

In some embodiments, B is selected from —C(R⁶)₃ and each R⁶ is independently selected at each occurrence from (i), (ii), and (iii):
(i) hydrogen, halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle and 3- to 10-membered heterocycle, each of which is optionally substituted with one or more substituents independently selected from: halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN.

In some aspects, the structure of Formula (I′) is represented by the structure of Formula (I).

In some aspects, the present disclosure provides a compound represented by the structure of Formula (I):

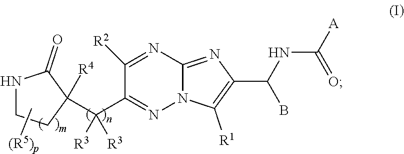

or a pharmaceutically acceptable salt thereof wherein:
A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
each R′ is selected from hydrogen, halogen, —OR^{18A}, —N(R^{18A})₂, —C(O)R^{18A}, —C(O)N(R^{18A})₂, —NO₂, —CN; and C₁₋₆ alkyl optionally substituted with one or more from halogen, —OR^{18A}, —N(R^{18A})₂, —C(O)R^{18A}, —C(O)N(R^{18A})₂, —NO₂, and —CN;
each R″ is selected from hydrogen, halogen, —OR^{19A}, —N(R^{19A})₂, —C(O)R^{19A}, —C(O)N(R^{19A})₂, —NO₂, —CN; and C₁₋₆ alkyl optionally substituted with one or more from halogen, —OR^{19A}, —N(R^{19A})₂, —C(O)R^{19A}, —C(O)N(R^{19A})₂, —NO₂, and —CN;
B is selected from —CH(R⁶)₂ and C₃₋₁₀ carbocycle, wherein the C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹, —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R² is selected from:
   hydrogen, halogen, —OR¹⁵, —SR¹⁵, —N(R¹⁵)₂, —C(O)R¹⁵, —C(O)N(R¹⁵)₂, —N(R¹⁵)C(O)R¹¹, —N(R¹⁵)S(O)₂R¹⁵, —C(O)OR¹⁵, —OC(O)R¹⁵, —S(O)R¹⁵, —S(O)₂R¹⁵, —NO₂, —CN;
   C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN; and
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
R¹¹, R¹², R¹³, R¹⁴, R¹⁵, R^{15A}, R¹⁶, R¹⁷, R^{18A}, and R^{19A} are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2;
p is selected 1, 2, 3 and 4; and z is selected from 0, 1, and 2.

In some embodiments, for the compound or salt of Formula (I′) or (I), z is 0.

In some embodiments, for the compound or salt of Formula (I′) or (I), z is 1.

In some embodiments, for the compound or salt of Formula (I′) or (I), z is 2.

In some embodiments, for the compound or salt of Formula (I′) or (I), R′ is selected from hydrogen, halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl. In some embodiments, R′ is selected from hydrogen, halogen, C₁₋₆ alkyl, and C₁₋₆ haloalkyl. In some embodiments, R′ is hydrogen. In some embodiments, R′ is C₁₋₆ alkyl. In some embodiments, R is C₁₋₆ haloalkyl.

In some embodiments, for the compound or salt of Formula (I′) or (I), R″ is selected from hydrogen, halogen, —OR^{18A}, —N(R^{18A})₂, —C(O)R^{18A}, —C(O)N(R^{18A})₂, —NO₂, —CN, C₁₋₆ alkyl, and C₁₋₆ haloalkyl. In some embodiments, R″ is selected from hydrogen, halogen, C₁₋₆ alkyl, and C₁₋₆ haloalkyl. In some embodiments, R″ is hydrogen. In some embodiments, R″ is C₁₋₆ alkyl. In some embodiments, R″ is C₁₋₆ haloalkyl.

In some embodiments, for the compound or salt of Formula (I′) or (I), R′ and R″ are each independently selected from hydrogen, halogen, C₁₋₆ alkyl, and C₁₋₆ haloalkyl. In some embodiments, R′ and R″ are each independently selected from hydrogen, C₁₋₆ alkyl, and C₁₋₆ haloalkyl. In some embodiments, R′ and R″ are each independently hydrogen.

In some embodiments, for the compound or salt of Formula (I′) or (I), A is selected —O—(CR′R″)_{z}(3- to 10-membered heterocycle) and —O—(CR′R″)_{z}(C₃₋₁₀ carbocycle), the 3 to 10-membered heterocycle and C₃₋₁₀ carbocycle are each optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I′) or (I), A is —O—(CR′R″)_{z}(3- to 10-membered heterocycle), the 3 to 10-membered heterocycle is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is —O—(CR′R″)_{z}(3- to 10-membered heterocycle), the 3 to 10-membered heterocycle is optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, —CN; and C₁₋₆ alkyl optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN.

In some embodiments, for the compound or salt of Formula (I′) or (I), A is —O—(CR′R″)_{z}(5-membered heteroaryl), the 5-membered heteroaryl is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is —O—(CR′R″)_{z}(5-membered heteroaryl), the 5-membered heteroaryl is optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, —CN; and C₁₋₆ alkyl optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN.

In some embodiments, for the compound or salt of Formula (A), (I′), or (I), A is —O—(CR′R″)_{z}(5-membered heteroaryl), the 5-membered heteroaryl is optionally substituted with one or more substituents independently selected from halogen, —OR¹¹, —N(R¹¹)₂, —C(O)R¹¹, —NO₂, —CN; and C₁₋₆ alkyl optionally substituted with one or more substituents independently selected from: halogen, —OR¹¹, —N(R¹¹)₂, —C(O)R¹¹, —NO₂, and —CN. In some embodiments, A is —O—(CR′R″)_{z}(5-membered heteroaryl), the 5-membered heteroaryl of —O—(CR′R″)_{z}(5-membered heteroaryl) is selected from pyrrolyl, furanyl, thiophenyl, imidazolyl, pyrazolyl, oxadiazolyl, isoxadiazolyl, triazolyl, and tetrazolyl, each of which is optionally substituted with one or more substituents independently selected from halogen, —OR¹¹, —N(R¹¹)₂, —C(O)R¹¹, —NO₂, —CN; and C₁₋₆ alkyl optionally substituted with one or more substituents independently selected from: halogen, —OR¹¹, —N(R¹¹)₂, —C(O)R¹¹, —NO₂, and —CN. In some embodiments, A

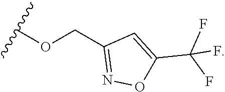 [SMILES: *OCc1cc(C(F)(F)F)on1]

In some embodiments, for the compound or salt of Formula (I′) or (I), R² is C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle. In some embodiments, R² is C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is C₁₋₁₀ alkyl optionally substituted with one or more substituents independently selected from C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R¹⁵, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₂₋₁₀ alkenyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle. In some embodiments, R² is C₂₋₁₀ alkenyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is C₂₋₁₀ alkenyl optionally substituted with one or more substituents independently selected from C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN.

In some embodiments, for the compound or salt of Formula (I′) or (I), R² is C₁₋₁₀ alkyl optionally substituted with one or more substituents independently selected from C₃₋₁₀ carbocycle and 3- to 10-membered heterocycle each of which is optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more substituents independently selected from C₃₋₈ cycloalkyl and 3- to 8-membered heterocycloalkyl, each of which is optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more substituents independently selected from C₃₋₈ cycloalkyl and 3- to 8-membered heterocycloalkyl, each of which is optionally substituted with one or more substituents independently selected from halogen, —OR^{15A}, —N(R^{15A})₂, —C(O)R^{15A}, —C(O)N(R^{15A})₂, —N(R^{15A})C(O)R^{15A}, —C(O)OR^{15A}, —OC(O)R^{15A}, —NO₂, and —CN.

In some embodiments, for the compound or salt of Formula (I′) or (I), R² is C₁₋₆ alkyl optionally substituted with one or more 3- to 8-membered heterocycloalkyl optionally substituted with one or more substituents independently selected from halogen, —OR^{15A}, —N(R^{15A})₂, —C(O)R^{15A}, —C(O)N(R^{5A})₂, —N(R^{15A})C(O)R^{15A}, —C(O)OR^{15A}, —OC(O)R^{15A}, —NO₂, and —CN. In some embodiments, R² is C₁₋₆ alkyl optionally substituted with one or more substituents independently selected from oxtanyl, tetrahydrofuranyl, tetrahydropyranyl, dioxoanyl, and morpholinyl, each of which is optionally substituted with one or more substituents independently selected from halogen, —OR^{15A}, —N(R^{15A})₂, —C(O)R^{15A}, —C(O)N(R^{15A})₂, —N(R^{15A})C(O)R^{15A}, —C(O)OR^{15A}, —OC(O)R^{15A}, —NO₂, and —CN. In some embodiments, R² is C₁₋₆ alkyl optionally substituted with one or more substituents independently selected from oxtanyl, tetrahydrofuranyl, tetrahydropyranyl, dioxoanyl, and morpholinyl. In some embodiments, R² is selected from:

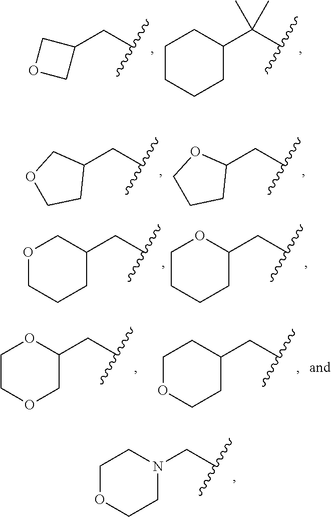

In some embodiments, for the compound or salt of Formula (I′) or (I), R² is C₁₋₁₀ alkyl optionally substituted with one or more substituents independently selected from C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is selected from methyl, ethyl, propyl, and isopropyl, each of which is optionally substituted with one or more substituents independently selected from C₃₋₆ carbocycle and 3- to 6-membered heterocycle, the C₃₋₆ carbocycle and 3- to 6-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more substituents independently selected from C₃₋₆ carbocycle and 3- to 6-membered heterocycle, the C₃₋₆ carbocycle and 3- to 6-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN.

In some embodiments, for the compound or salt of Formula (I′) or (I), R² is C₁₋₁₀ alkyl optionally substituted with one or more substituents independently selected from azetidinyl, pyrrolidinyl, piperidinyl, piperazinyl, oxtanyl, tetrahydrofuranyl, tetrahydropyranyl, morpholinyl, and imidazolyl, each of which is optionally substituted with one or more substituents independently selected from halogen, —OR¹⁵A, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}) and —CN. In some embodiments, R² is C₁₋₆ alkyl optionally substituted with one or more substituents independently selected from azetidinyl, pyrrolidinyl, piperidinyl, piperazinyl, oxtanyl, tetrahydrofuranyl, tetrahydropyranyl, morpholinyl, and imidazolyl, each of which is optionally substituted with one or more substituents independently selected from halogen, —OR¹¹, —N(R¹¹)₂, —NO₂, and —CN. In some embodiments, R² is selected from:

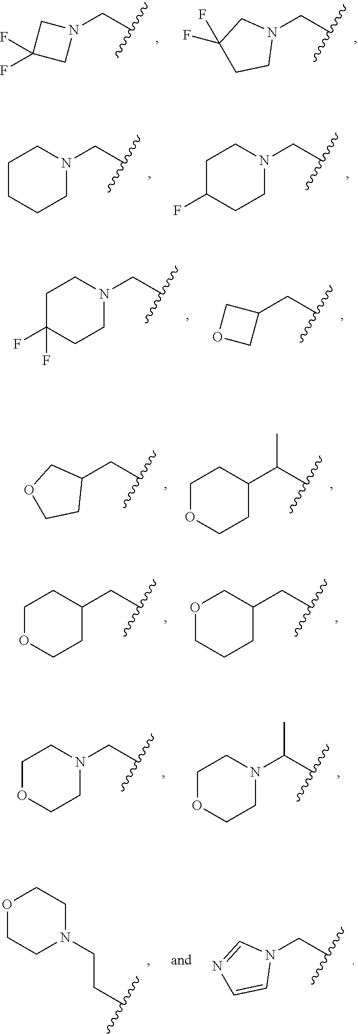

In some embodiments, for the compound or salt of Formula (I′) or (I), R² is C₁₋₁₀ alkyl optionally substituted with one or more substituents independently selected from 3- to 8-membered monocyclic heterocycle and 5- to 10-membered bicyclic heterocycle, are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more 5- to 10-membered bicyclic heterocycle optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² is C₁₋₆ alkyl optionally substituted with one or more 5- to 10-membered fused heterocycle selected from 5- to 10-membered fused heterocycle, 5- to 10-membered bridged heterocycle, or 5- to 10-membered fused heterocycle, each of which is optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO², ═O, ═S, ═N(R^{15A}), and —CN. In some embodiments, R² is selected from:

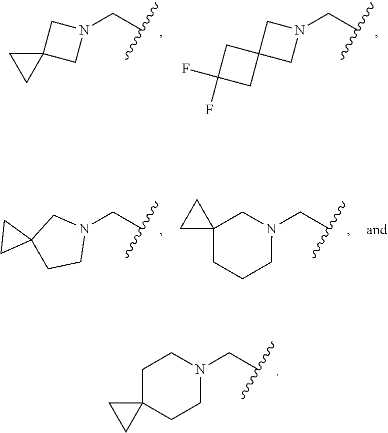

In some embodiments, for the compound or salt of Formula (I′) or (I), R² is C₁₋₆ alkyl optionally substituted with one or more 5- to 10-membered bridged heterocycle optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{45A}, —S(O)₂R^{15A}, —NO₂, ═O, ═S, ═N(R^{15A}), and —CN. In some embodiments, R² is

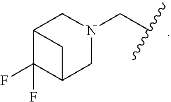 [SMILES: *CN1CC2CC(C1)C2(F)F]

In some embodiments, for the compound or salt of Formula (I′) or (I), R² is C₂₋₆ alkenyl optionally substituted with one or more substituents independently selected from halogen, —OR¹⁵, —N(R¹⁵)₂, —C(O)R¹⁵, —NO₂, and —CN. In some embodiments, R² is selected from ethenyl, propenyl, butenyl, each of which is optionally substituted with one or more substituents independently selected from halogen, —OR¹⁵, —N(R¹⁵)₂, —C(O)R¹⁵, —NO₂, and —CN. In some embodiments, R² is

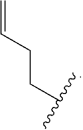 [SMILES: *CCC=C]

In some embodiments, for the compound or salt of Formula (I′) or (I), R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, and —CN. In some embodiments, R² is selected from hydrogen, halogen, —OR¹⁵, —N(R¹⁵)₂, —C(O)R¹⁵, —C(O)N(R¹⁵)₂, —N(R¹⁵)C(O)R¹⁵, —NO₂, and —CN. In some embodiments, R² is —N(R¹⁵)₂. wherein each R¹⁵ is independently selected from C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN.

In some embodiments, for the compound or salt of Formula (I′) or (I), R² is —N(R¹⁵)₂. wherein each R¹⁵ is independently selected from C₁₋₆ alkyl optionally substituted with one or more substituents independently selected from halogen, —NH₂, —CN, and 4-membered heterocycle, wherein each 4-membered heterocycle are optionally substituted with one or more substituents independently selected from: halogen, —OH, C₁₋₆ alkyl, —O—C₁₋₆ alkyl, —O—C₁₋₆ haloalkyl, —NH₂, —NO₂, ═O, and —CN. In some embodiments, R² is

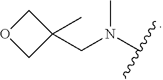 [SMILES: *N(C)CC1(C)COC1]

In some aspects, the structure of Formula (I′) or (I) is represented by the structure of Formula (II):

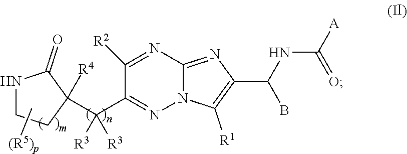

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
   C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —CH(R⁶)₂ and C₃₋₁₀ carbocycle,
   wherein the C₃₋₁₀ carbocycle is optionally substituted with one or more substituents independently selected from:
      halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
      C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle, wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁶ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, —CN,
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle and 3- to 10-membered heterocycle, each of which is optionally substituted with one or more substituents independently selected from:
      halogen, —OR¹³, —SR¹³, —N(R¹³)₂, —C(O)R¹³, —C(O)N(R¹³)₂, —N(R¹³)C(O)R, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —C(O)OR¹³, —OC(O)R¹³, —S(O)R¹³, —S(O)₂R¹³, —NO₂, ═O, ═S, ═N(R¹³), and —CN;
R¹ is selected from:
   hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, —CN; and
   C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN;
R² is selected from:
   hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹, —OC(O)R¹⁵, —S(O)R¹⁵, —S(O)₂R¹⁵, —NO₂, ═O, ═S, ═N(R¹⁵), and —CN; and
   C₃₋₁₀ carbocycle and 3- to 10-membered heterocycle, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ and R⁴ is independently selected at each occurrence from:
   hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and
   C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R⁵ is independently selected at each occurrence from:
   halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and
   C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
R¹¹, R¹², R¹³, R¹⁴, R¹⁵, R¹⁶, and R¹⁷ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
m is selected from 1, 2, and 3;
n is selected from 1 and 2; and
p is selected 1, 2, 3, and 4.

In some embodiments, for the compound or salt of Formula (A), (I′), (I), or (II), m is 1.

In some embodiments, for the compound or salt of Formula (A), (I′), (I), or (II), m is 2.

In some embodiments, for the compound or salt of Formula (A), (I′), (I), or (II), m is 3.

In certain embodiments, the structure of Formula (I′), (I), or (II), is represented by Formula (II-a):

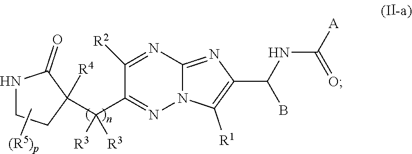

In certain embodiments, the structure of Formula (I), (I), or (II), is represented by the structure of Formula (II-b):

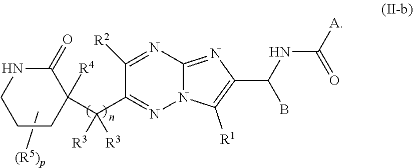

In some embodiments, for the compound or salt of Formula (A), (I′), (I), (II), (II-a), or (II-b), p is selected from 0, 1, 2, 3, and 4. In some embodiments, p is selected from 0, 1, 2, and 3. In some embodiments, p is selected from 0, 1, and 2. In some embodiments, p is selected from 0, and 1. In some embodiments, p is selected from 1, 2, 3, and 4. In some embodiments, p is selected from 2, 3, and 4. In some embodiments, p is selected from 3 and 4. In some embodiments, p is 0. In some embodiments, p is 1. In some embodiments, p is 2. In some embodiments, p is 3. In some embodiments, p is 4.

In some embodiments, for the compound or salt of Formula (A), (I′), (I), (II), (II-a), or (II-b), each R⁵ is independently selected at each occurrence from:
halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, ═O, ═S, ═N(R¹⁷), and —CN.

In some embodiments, for the compound or salt of Formula (A), (I′), (I), (II), (II-a), or (II-b), each R⁵ is independently selected at each occurrence from:
halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, C(O)OR¹⁷, —OC(O)R¹⁷, —S(O)R¹⁷, —S(O)₂R¹⁷, —NO₂, and —CN; and
C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, C(O)OR¹⁷, —OC(O)R¹⁷, —S(O)R¹⁷, —S(O)₂R¹⁷, —NO₂, ═O, ═S, ═N(R¹⁷), and CN.

In some embodiments, for the compound or salt of Formula (A), (I′), (I), (II), (II-a), or (II-b), each R⁵ is independently selected at each occurrence from C₁₋₆ alkyl optionally substituted with one or more substituents independently selected from halogen, —OR¹⁷, —N(R¹⁷)₂, —C(O)R¹⁷, —C(O)N(R¹⁷)₂, —NO₂, ═O, and CN. In some embodiments, each R⁵ is independently selected at each occurrence from methyl, ethyl, propyl, and isopropyl, each of which is optionally substituted with one or more halogen, —OR¹⁷, —N(R¹⁷)₂, and —CN. In some embodiments, each R⁵ is —CF₃. In some embodiments, R⁵ is C₁₋₆haloalkyl. In some embodiments, R⁵ is —CF₃.

In some embodiments, for the compound or salt of Formula (A), (I′), (I), (II), (II-a), or (II-b), each R⁵ is independently selected at each occurrence from halogen, —OR¹⁷, —SR¹⁷, —N(R¹⁷)₂, —C(O)R¹⁷, —NO₂, and —CN. In some embodiments, each R⁵ is independently selected at each occurrence from halogen. In some embodiments, each R⁵ is —F.

In some embodiments, for the compound or salt of Formula (A), (I′), (I), (II), (II-a), or (II-b),

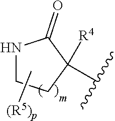

is selected from

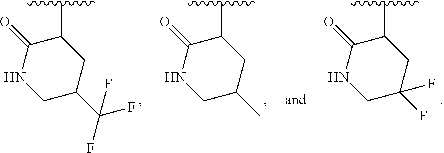

In some embodiments, for the compound or salt of Formula (A), (I′), (I), (II), (II-a), or (II-b), each R³ and R⁴ is independently selected at each occurrence from: hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ and R⁴ is independently selected at each occurrence from: hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, and —CN. In some embodiments, each R³ and R⁴ is independently selected at each occurrence from: C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ and R⁴ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, and —OC(O)R¹⁶. In some embodiments, each R³ and R⁴ is independently selected at each occurrence from hydrogen, halogen, —OR¹⁶, —SR¹⁶, —N(R¹⁶)₂, —C(O)R⁶, —C(O)N(R⁶)₂, —C(O)OR⁶, and —OC(O)R¹⁶. In some embodiments, each R³ and R⁴ is independently selected at each occurrence from hydrogen and halogen. In some embodiments, each R³ and R⁴ is hydrogen. In some embodiments, R³ is hydrogen. In some embodiments, R⁴ is hydrogen.

In some embodiments, for the compound or salt of Formula (A), (I′), (I), (II), (II-a), or (II-b), n is selected from 1 and 2. In some embodiments, n is 1. In some embodiments, n is 2.

In some embodiments, for the compound or salt of Formula (A), (I′), (I), (II), (II-a), or (II-b), A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN; and
C₃₋₆ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (A), (I′), (I), (II), (II-a), or (II-b), A is 5- to 6-membered heteroaryl optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN;
C₁₋₁₀ alkyl optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN; and
C₃₋₁₀ carbocycle optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN.

In some embodiments, for the compound or salt of Formula (A), (I′), (I), (II), (II-a), or (II-b), A is selected from 5- to 6-membered heteroaryl and C₃₋₆ carbocycle, any of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, and —CN. In some embodiments, A is 5- to 6-membered heteroaryl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN.

In some embodiments, for the compound or salt of Formula (A), (I′), (I), (II), (II-a), or (II-b), A is selected from 5- to 6-membered heteroaryl and C₃₋₆ carbocycle, any of which is optionally substituted with C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is 5- to 6-membered heteroaryl optionally substituted with C₃₋₁₀ carbocycle optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN. In some embodiments, A is selected from 5- to 6-membered heteroaryl optionally substituted with C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, and —CN. In some embodiments, A is selected from 5- to 6-membered heteroaryl optionally substituted with C₁₋₁₀ alkyl optionally substituted with halogen.

In some embodiments, for the compound or salt of Formula (A), (I′), (I), (II), (II-a), or (II-b), A is selected from 5- to 6-membered heteroaryl and C₃₋₆ carbocycle, any of which is optionally substituted with C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN; and C₁₋₁₀ alkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is selected from 5- to 6-membered heteroaryl optionally substituted with C₃₋₁₀ carbocycle optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, and —S(O)₂R¹¹. In some embodiments, A is selected from 5- to 6-membered heteroaryl optionally substituted with C₃₋₁₀ carbocycle optionally substituted with halogen.

In some embodiments, for the compound or salt of Formula (A), (I′), (I), (II), (II-a), or (II-b), A is selected from pyrazolyl, imidazolyl, oxazolyl, isoxazolyl, oxadiazolyl, triazolyl, and tetrazolyl, each of which is optionally substituted with one or more C₁₋₁₀ alkyl optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN. In some embodiments, A is selected from pyrazolyl, isoxazolyl, and oxadiazolyl, each of which is optionally substituted with one or more C₁₋₃ alkyl optionally substituted with one or more substituents independently selected from halogen, —OR¹¹, —N(R¹¹)₂, —NO₂, and CN. In some embodiments, A is selected from pyrazolyl, isoxazolyl, and oxadiazolyl, each of which is optionally substituted with methyl, ethyl, propyl, and isopropyl, each of which is optionally substituted with one or more substituents independently selected from halogen, —OR¹¹, —N(R¹¹)₂, —NO₂, and —CN. In some embodiments, A is selected from:

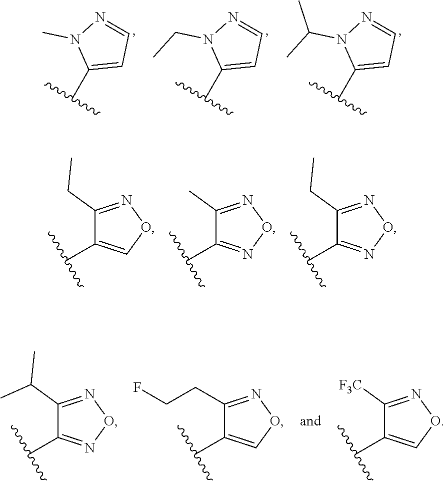

In some embodiments, A is selected from:

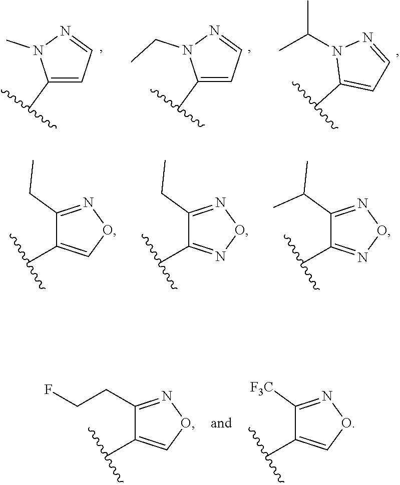

In some embodiments, A is selected from:

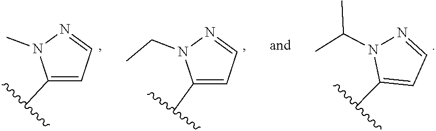

In some embodiments, A is selected from:

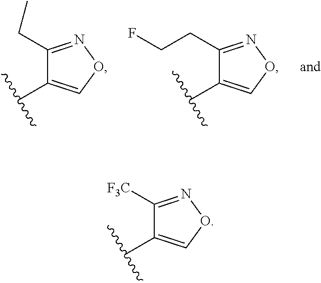

In some embodiments, A is selected from:

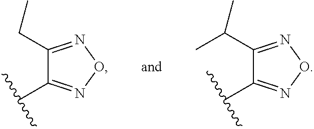

In some embodiments, A is selected from:

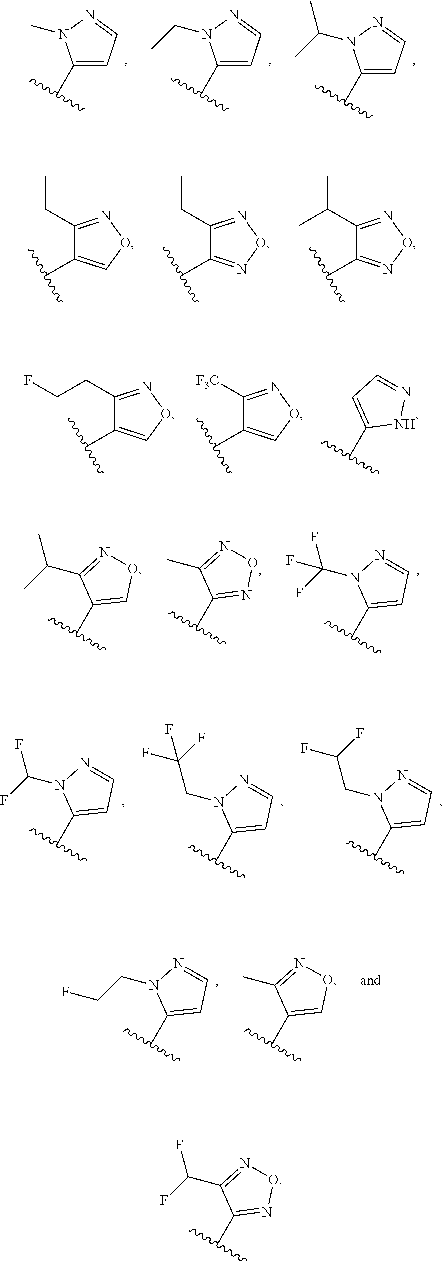

In some embodiments, A is selected from:

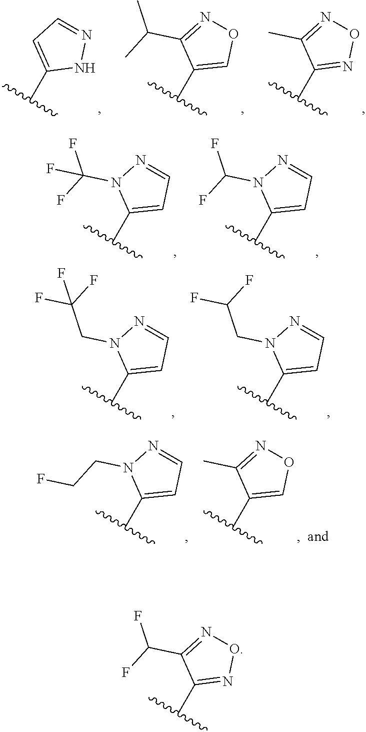

In some embodiments, A is

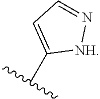 [SMILES: *c1ccn[nH]1]

In some embodiments, A is selected from:

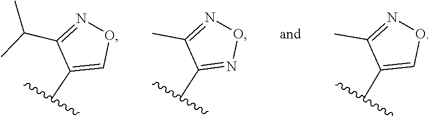

In some embodiments,

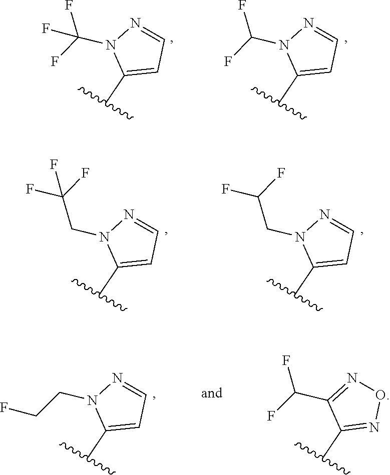 [SMILES: *c1ccnn1C(F)(F)F ; *c1ccnn1C(F)F ; *c1ccnn1CC(F)(F)F ; *c1ccnn1CC(F)F ; *c1ccnn1CCF ; *c1nonc1C(F)F]

In some embodiments, for the compound or salt of Formula (A), (I′), (I), (II), (II-a), or (II-b), A is selected from pyrazolyl, imidazolyl, oxazolyl, isoxazolyl, oxadiazolyl, triazolyl, and tetrazolyl, each of which is optionally substituted with one or more C₃₋₁₀ carbocycle optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN. In some embodiments, A is selected from pyrazolyl, isoxazolyl, and oxadiazolyl, any of which is optionally substituted with C₃₋₆ saturated carbocycle optionally substituted with one or more substituents independently selected from halogen, —OR¹¹, —N(R¹¹)₂, —NO₂, and —CN. In some embodiments, A is selected from pyrazolyl, isoxazolyl, and oxadiazolyl, any of which is optionally substituted with cyclopropyl, cyclobutyl, cyclopentyl, and cyclohexyl, any of which is optionally substituted with one or more substituents independently selected from halogen, —OR¹¹, —N(R¹¹)₂, —NO₂, and —CN. In some embodiments, A is selected from:

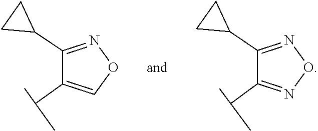

In some embodiments, A is selected from

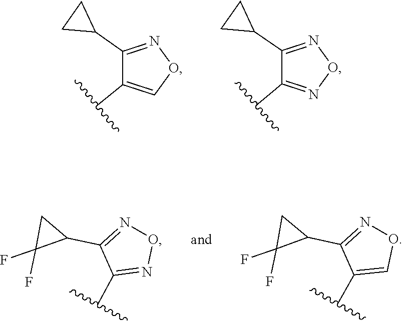 [SMILES: *c1conc1C1CC1 ; *c1nonc1C1CC1 ; *c1nonc1C1CC1(F)F ; "and" ; *c1conc1C1CC1(F)F]

In some embodiments, A is selected from

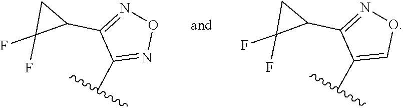 [SMILES: *c1nonc1C1CC1(F)F ; "and" ; *c1conc1C1CC1(F)F]

In some embodiments, for the compound or salt of Formula (A), (I′), (I), or (II), A is selected from pyrimidinyl, pyrazolyl, imidazolyl, oxazolyl, isoxazolyl, oxadiazolyl, triazolyl, and tetrazolyl, each of which is optionally substituted with one or more C₁₋₁₀ alkyl optionally substituted with one or more substituents independently selected from halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, and —CN. In some embodiments, A is selected from is wherein A is selected from pyrimidinyl, pyrazolyl, imidazolyl, triazolyl, isoxazolyl, and oxadiazolyl, each of which is optionally substituted with one or more C₁₋₁₀ alkyl optionally substituted with one or more substituents independently selected from halogen, —OR¹¹, —N(R¹¹)₂, —NO₂, and CN. In some embodiments, A is selected from is wherein A is selected from pyrimidinyl, pyrazolyl, imidazolyl, triazolyl, isoxazolyl, and oxadiazolyl, each of which is optionally substituted with methyl, ethyl, propyl, tert-butyl, and isopropyl, each of which is optionally substituted with one or more substituents independently selected from halogen, —OR¹¹, —N(R¹¹)₂, —NO₂, and —CN. In some embodiments, A is selected from

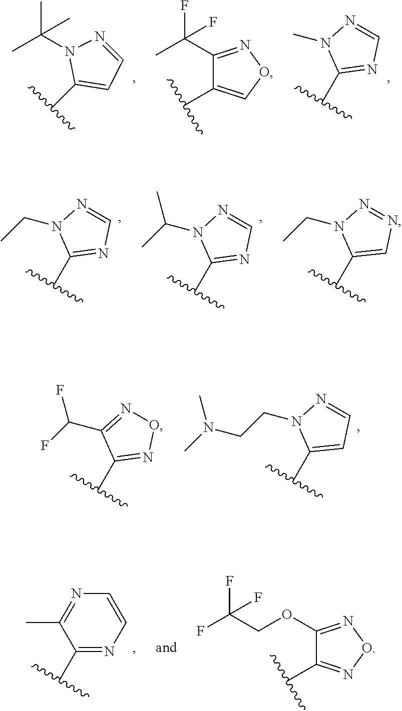

In some embodiments, for the compound or salt of Formula (A), (I′), (I), (II), A is 5- to 6-membered heteroaryl optionally substituted with one or more C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is selected from pyrimidinyl, pyrazolyl, imidazolyl, oxazolyl, isoxazolyl, oxadiazolyl, triazolyl, and tetrazolyl, each of which is optionally substituted with one or more C₁₋₁₀ alkyl optionally substituted with one or more substituents selected from C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN. In some embodiments, A is selected from pyrazolyl, imidazolyl, oxadiazolyl, each of which is optionally substituted with one or more C₁₋₃ alkyl optionally substituted with one or more substituents selected from C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —NO₂, and CN. In some embodiments, A is selected from pyrazolyl, imidazolyl, oxadiazolyl, each of which is optionally substituted with one or more C₁₋₃ alkyl optionally substituted with one or more substituents selected from azetidinyl, pyrrolidinyl, piperazinyl, and morpholinyl; wherein the azetidinyl, pyrrolidinyl, piperazinyl, and morpholinyl are each optionally substituted with one or more substituents selected from: halogen, —OR¹¹, —N(R¹¹)₂, —NO₂, and CN. In some embodiments, A is selected from

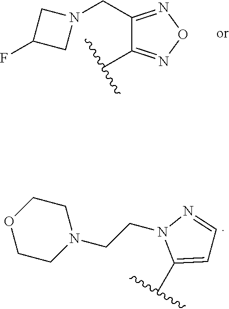 [SMILES: *c1nonc1CN1CC(F)C1 ; "or" ; *c1ccnn1CCN1CCOCC1]

In some embodiments, for the compound or salt of Formula (A), (I′), (I), (II), (II-a), or (II-b), A is C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R′)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (A), (I′), (I), (II), (II-a), or (II-b), A is C₃₋₆ carbocycle optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN. In some embodiments, A is C₃₋₆ saturated carbocycle optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN. In some embodiments, A is C₃₋₆ cycloalkyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN. In some embodiments, A is selected from cyclopropyl, cyclobutyl, and cyclopentyl, each of which is optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN. In some embodiments A is C₃ carbocycle optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, and —CN. In some embodiments, A is

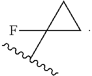 [SMILES: *C1(F)CC1]

In some embodiments, for the compound or salt of Formula (A), (I′), (I), (II), (II-a), or (II-b), B is optionally substituted C₃₋₁₀ carbocycle. In some embodiments, B is optionally substituted C₃₋₁₀ saturated carbocycle. In some embodiments, B is optionally substituted C₃₋₁₀ unsaturated carbocycle. In some embodiments, B is selected from C₃₋₄ carbocycle, C₃₋₅ carbocycle, C₃₋₆ carbocycle, C₃₋₇ carbocycle, C₃₋₈ carbocycle, C₃₋₉ carbocycle, and C₃₋₁₀ carbocycle, each of which is optionally substituted with substituents as defined in Formula (I′) or (I). In some embodiments, B is selected from C₃ carbocycle, C₄ carbocycle, C₅ carbocycle, C₆ carbocycle, C₇ carbocycle, C₈ carbocycle, C₉ carbocycle, and C₁₀ carbocycle, each of which is optionally substituted with substituents as defined in Formula (I′) or (I). In some embodiments, B is selected from C₃₋₈ monocyclic carbocycle and C₅₋₁₀ bicyclic carbocycle, each of which is optionally substituted with substituents as defined in Formula (I′) or (I). In some embodiments, B is C₅₋₁₀ bicyclic carbocycle, the C₅₋₁₀ bicyclic carbocycle is selected from C₅₋₁₀ fused carbocycle, C₅₋₁₀ bridged carbocycle, and C₅₋₁₀ spirocyclic carbocycle, each of which is optionally substituted with substituents as defined in Formula (I′) or (I).

In some embodiments, for the compound or salt of Formula (A), (I′), (I), (II), (II-a), or (II-b), B is selected from —CH(R⁶)₂ and C₃₋₁₀ carbocycle, wherein the C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN.

In some embodiments, for the compound or salt of Formula (A), (I′), (I), (II), (II-a), or (II-b), B is selected from —CH(R⁶)₂ and C₃₋₁₀ carbocycle, wherein the C₃₋₁₀ carbocycle is optionally substituted with one or more substituents independently selected from: halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), and —CN.

In some embodiments, for the compound or salt of Formula (A), (I′), (I), (II), (II-a), or (II-b), B is —CH(R⁶)₂.

In some embodiments, for the compound or salt of Formula (A), (I′), (I), (II), (II-a), or (II-b), B is selected from —CH(R⁶)₂ and C₃₋₁₀ carbocycle, wherein the C₃₋₁₀ carbocycle is optionally substituted with one or more substituents independently selected from: halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), and —CN.

In some embodiments, for the compound or salt of Formula (A), (I′), (I), (II), (II-a), or (II-b), B is C₃₋₁₀ carbocycle, wherein the C₃₋₁₀ carbocycle is optionally substituted with one or more substituents independently selected from: halogen, C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN. In some embodiments, B is C₆₋₁₀ carbocycle optionally substituted with one or more substituents independently selected from: halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, CN, and C₁₋₆ alkyl optionally substituted with one or more substituents independently selected from: halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, and CN. In some embodiments, B is selected from cyclohexyl and cycloheptyl, each of which is optionally substituted with one or more substituents independently selected from: halogen, —OR¹², —N(R¹²)₂, —NO₂, ═O, CN, and C₁₋₆ alkyl optionally substituted with one or more substituents independently selected from: halogen, —OR¹², —N(R¹²)₂, —NO₂, ═O, and CN. In some embodiments, B is selected from:

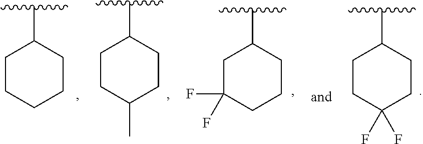

In some embodiments, B is selected from:

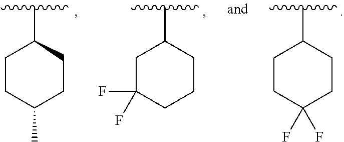

In some embodiments, B is selected from:

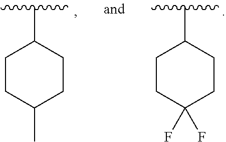

In some embodiments, B is selected from:

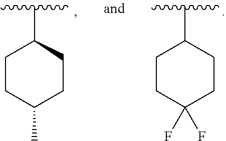

In some embodiments, B is selected from:

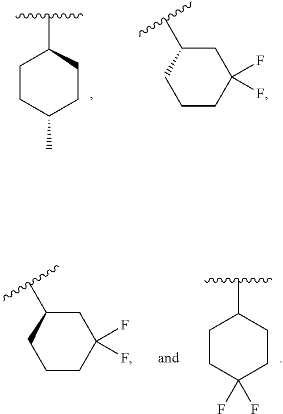

In some embodiments, B is

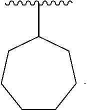 [SMILES: *C1CCCCCC1]

In some embodiments, for the compound or salt of Formula (A), (I′), (I), (II), (II-a), or (II-b), B is C₄₋₁₀ carbocycle optionally substituted with one or more substituents independently selected from
halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, CN, and
C₃₋₆ carbocycle optionally substituted with one or more substituents independently selected from: halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, and CN.

In some embodiments, for the compound or salt of Formula (A), (I′), (I), (II), (II-a), or (II-b), B is selected from C₄₋₇ monocyclic carbocycle, C₅₋₁₀ fused carbocycle, and C₅₋₁₀ spirocyclic carbocycle, any of which is optionally substituted with one or more substituents independently selected from
halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, CN, and
C₃₋₆ carbocycle optionally substituted with one or more substituents independently selected from: halogen, —OR¹², —N(R¹²)₂, —C(O)R¹², —C(O)N(R¹²)₂, —N(R¹²)C(O)R¹², C(O)OR¹², —OC(O)R¹², —NO₂, ═O, and CN.

In some embodiments, for the compound or salt of Formula (A), (I′), (I), (II), (II-a), or (II-b), B is selected from cyclobutyl, bicyclo[3.1.0]hexanyl, and spiro[3.3]heptanyl, any of which is optionally substituted with one or more substituents independently selected from halogen, —OR¹², —N(R¹²)₂, —NO₂, ═O, CN, and C₃₋₆ carbocycle optionally substituted with one or more substituents independently selected from halogen, —OR¹², —N(R¹²)₂, —NO₂, ═O, and CN. In some embodiments, B is selected from:

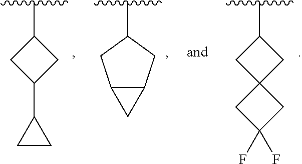

In some embodiments, for the compound or salt of Formula (A), (I′), (I), (II), (II-a), or (II-b), B is selected from cyclobutyl, bicyclo[3.1.0]hexanyl, bicyclo[5.1.0]octanyl, and spiro[3.3]heptanyl, any of which is optionally substituted with one or more substituents independently selected from halogen, —OR¹², —N(R¹²)₂, —NO₂, ═O, CN, and C₃₋₆ carbocycle optionally substituted with one or more substituents independently selected from halogen, —OR¹², —N(R¹²)₂, —NO₂, ═O, and CN. In some embodiments, B is

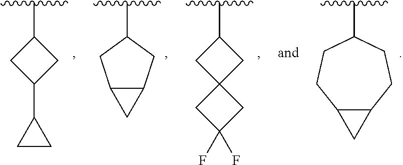

In some embodiments, for the compound or salt of Formula (A), (I′), (I), (II), (II-a), or (II-b), each R⁶ of —CH(R⁶)₂ is independently selected from:
halogen, —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, —CN;
C₁₋₆ alkyl optionally substituted with one or more substituents independently selected from halogen, —OR¹³, —N(R¹³)₂, —C(O)R¹³, —C(O)N(R¹³)₂, —N(R¹³)C(O)R¹³, —N(R¹³)S(O)₂R¹³, C(O)OR¹³, —OC(O)R¹³, —NO₂, ═O, ═S, ═N(R¹³), and CN; and
C₃₋₈ carbocycle optionally substituted with one or more substituents independently selected from: halogen, —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, ═O, ═S, ═N(R¹³), —CN; and C₁₋₆ alkyl optionally substituted with one or more substituents independently selected from halogen,
   —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, ═O, ═S, ═N(R¹³), and —CN.

In some embodiments, for the compound or salt of Formula (A), (I′), (I), (II), (II-a), or (II-b), each R⁶ of —CH(R⁶)₂ is independently selected from halogen, —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, and —CN.

In some embodiments, for the compound or salt of Formula (A), (I′), (I), (II), (II-a), or (II-b), each R⁶ of —CH(R⁶)₂ is independently selected from C₁₋₆ alkyl optionally substituted with one or more substituents independently selected from halogen, —OR¹³, —N(R¹³)₂, —C(O)R¹³, —C(O)N(R¹³)₂, —N(R¹³)C(O)R¹³, —N(R¹³)S(O)₂R¹³, C(O)OR¹³, —OC(O)R¹³, —NO₂, ═O, ═S, ═N(R¹³), and CN.

In some embodiments, for the compound or salt of Formula (A), (I′), (I), (II), (II-a), or (II-b), each R⁶ of —CH(R⁶)₂ is independently selected from C₃₋₈ carbocycle optionally substituted with one or more substituents independently selected from: halogen, —OR¹³, —N(R¹³)₂, —C(O)R³, —C(O)N(R¹³)₂, —N(R¹³)C(O)R¹³, —N(R¹³)S(O)₂R¹³, —C(O)OR¹³, —OC(O)R¹³, —NO₂, ═O, ═S, ═N(R¹³), —CN; and C₁₋₆ alkyl optionally substituted with one or more substituents independently selected from halogen, —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, ═O, ═S, ═N(R¹³), and —CN. In some embodiments, each R⁶ of —CH(R⁶)₂ is independently selected from C₃₋₇ saturated carbocycle optionally substituted with one or more substituents independently selected from: halogen, —OR¹³, —N(R¹³)₂, —NO₂, —CN; and C₁₋₆ alkyl optionally substituted with one or more substituents independently selected from: halogen, —OR¹³, —N(R¹³)₂, —NO₂, and —CN. In some embodiments, —CH(R⁶)₂ is

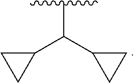 [SMILES: *C(C1CC1)C1CC1]

n some embodiments, for the compound or salt of Formula (A), (I), (II), (II-a), or (II-b), each R⁶ of —CH(R⁶)₂ is independently selected from:
C₁₋₆ alkyl optionally substituted with one or more substituents independently selected from halogen, —OR¹³, —N(R¹³)₂, —C(O)R¹³, —C(O)N(R¹³)₂, —N(R¹³)C(O)R¹³, —N(R¹³)S(O)₂R¹³, C(O)OR¹³, —OC(O)R¹³, —NO₂, ═O, ═S, ═N(R¹³), and CN; and
C₃₋₈ carbocycle optionally substituted with one or more substituents independently selected from: halogen, —OR¹³, —N(R¹³)₂, —C(O)R¹³, —C(O)N(R¹³)₂, —N(R¹³)C(O)R¹, —N(R¹³)S(O)₂R¹³, —C(O)OR¹³, —OC(O)R¹³, —NO₂, ═O, ═S, ═N(R¹³), —CN; and C₁₋₆ alkyl optionally substituted with one or more substituents independently selected from halogen, —OR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —NO₂, ═O, ═S, ═N(R¹³), and —CN.

In some embodiments, for the compound or salt of Formula (A), (I′), (I), (II), (II-a), or (II-b), each R⁶ of —CH(R⁶)₂ is independently selected from: C₁₋₆ alkyl optionally substituted with one or more substituents independently selected from halogen, —OR¹³, —N(R¹³)₂, —NO₂, ═O, and CN; and C₃₋₈ carbocycle optionally substituted with one or more substituents independently selected from: halogen, —OR¹³, —N(R¹³)₂, —C(O)OR¹³, —OC(O)R¹³, —NO₂, ═O, ═S, ═N(R¹³), —CN; and C₁₋₆ alkyl optionally substituted with one or more substituents independently selected from halogen, —OR¹³, —N(R¹³)₂, —NO₂, ═O, and —CN. In some embodiments, each R⁶ of —CH(R⁶)₂ is independently selected from C₁₋₆ alkyl optionally substituted with halogen, —OR¹³, —NO₂, —CN, and C₃₋₆ carbocycle optionally substituted with one or more substituents independently selected from halogen, —OR¹³, —NO₂, —CN, and C₁₋₆ alkyl optionally substituted with one or more substituents independently selected from halogen, —OR¹³, —NO₂, and —CN. In some embodiments, —CH(R⁶)₂ is selected from:

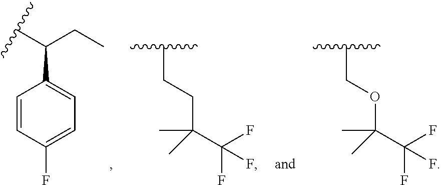

In some embodiments, for the compound or salt of Formula (A), (I′), (I), (II), (II-a), or (II-b), R¹ is selected from hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, and —CN. In some embodiments, R¹ is selected from hydrogen, halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, and —OC(O)R¹⁴. In some embodiments, R¹ is selected from hydrogen, halogen, —OR¹⁴, —SR¹⁴, —N(R¹⁴)₂, —C(O)R¹⁴, —C(O)N(R¹⁴)₂, and —OC(O)R¹⁴. In some embodiments, R¹ is hydrogen.

In some embodiments, for the compound or salt of Formula (A), (I′), (I), (II), (II-a), or (II-b), R² is selected from:
hydrogen, halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, CN;
C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, ═O, CN; and
3- to 10-membered saturated heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, ═O, and —CN.

In some embodiments, for the compound or salt of Formula (A), (I′), (I), (II), (II-a), or (II-b), R² is selected from: hydrogen, halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, and CN. In some embodiments, R² is selected from: hydrogen, halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, and —OC(O)R¹⁵. In some embodiments, R² is selected from: hydrogen, halogen, —OR¹⁵, and —N(R¹⁵)₂. In some embodiments, R² is hydrogen.

In some embodiments, for the compound or salt of Formula (A), (I′), (I), (II), (II-a), or (II-b), R² is selected from: C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, ═O, CN. In some embodiments, R² is selected from methyl, ethyl, propyl, and isopropyl, each of which is optionally substituted with one or more substituents independently selected from halogen, —OR¹⁵, —NO₂, ═O, and —CN. In some embodiments, R² is selected from methyl, ethyl, propyl, and isopropyl, each of which is optionally substituted with one or more halogen atoms. In some embodiments, R² is selected from

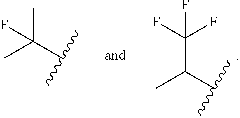

In some embodiments, R² is

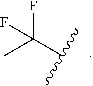 [SMILES: *C(C)(F)F]

In some embodiments, R² is selected from methyl, ethyl, propyl, and isopropyl. In some embodiments, R² is selected from

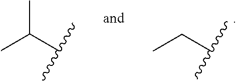

In some embodiments, R² is selected from methyl, ethyl, propyl, isopropyl, and tert-butyl, each of which is optionally substituted with one or more substituents independently selected from halogen, —OR¹⁵, —NO₂, ═O, and —CN. In some embodiments, R² is selected from

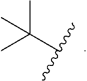 [SMILES: *C(C)(C)C ; "."]

In some embodiments, for the compound or salt of Formula (A), (I′), (I), (II), (II-a), or (II-b), R² is selected from methyl, ethyl, propyl, isopropyl, and tert-butyl, each of which is optionally substituted with one or more substituents independently selected from halogen, —N(R¹⁵)₂, —OR⁵, —NO₂, ═O, and —CN. In some embodiments, R² is selected from

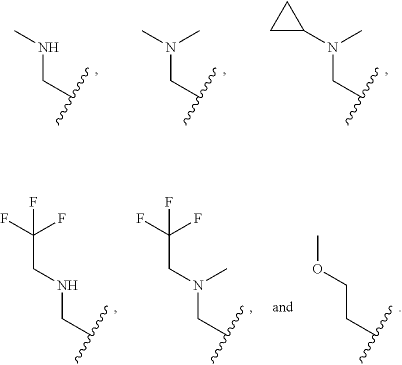

In some embodiments, for the compound or salt of Formula (A), (I′), (I), (II), (II-a), or (II-b), R² is selected from 3- to 10-membered saturated heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —NO₂, ═O, and —CN. In some embodiments, R² is selected from 3- to 10-membered saturated heterocycle optionally substituted with one or more substituents independently selected from halogen, —OR¹⁵, —N(R¹⁵)₂, —NO₂, ═O, and —CN. In some embodiments, R² is selected from tetrahydropyranyl, morpholinyl, 2-oxa-5-azabicyclo[2.2.1]heptanyl, 6-oxa-3-azabicyclo[3.1.1]heptanyl, 8-oxa-3-azabicyclo[3.2.1]octanyl, any of which is optionally substituted with one or more substituents independently selected from halogen, —OR¹⁵, —N(R¹⁵)₂, —NO₂, ═O, and —CN. In some embodiments, R² is

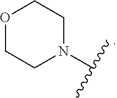 [SMILES: *N1CCOCC1]

In some embodiments, for the compound or salt of Formula (A), (I′), (I), (II), (II-a), or (II-b), R² is optionally substituted 3- to 10-membered heterocycle. In some embodiments, R² is optionally substituted 3- to 10-membered saturated heterocycle. In some embodiments, R² is optionally substituted 3- to 10-membered unsaturated heterocycle. In some embodiments, R² is selected from 3- to 4-membered heterocycle, 3- to 5-membered heterocycle, 3- to 6-membered heterocycle, 3- to 7-membered heterocycle, 3- to 8-membered heterocycle, 3- to 9-membered heterocycle, and 3- to 10-membered heterocycle, any of which is optionally substituted with substituents as defined in Formula (I′) or (I).

In some embodiments, R² is selected from 3-membered heterocycle, 4-membered heterocycle, 5-membered heterocycle, 6-membered heterocycle, 7-membered heterocycle, 8-membered heterocycle, 9-membered heterocycle, and 10-membered heterocycle, any of which is optionally substituted with substituents as defined in Formula (I′) or (I). In some embodiments, R² is selected from 3- to 8-membered monocyclic heterocycle and 5- to 10-membered bicyclic heterocycle, each of which is optionally substituted with substituents as defined in Formula (I′) or (I). In some embodiments, the 5- to 10-membered bicyclic heterocycle of R² is selected from 5- to 10-membered fused heterocycle, 5- to 10-membered bridged heterocycle, and 5- to 10-membered spirocyclic heterocycle, each of which is optionally substituted with substituents as defined in Formula (I′) or (I).

In some embodiments, for the compound or salt of Formula (A), (I′), (I), (II), (II-a), or (II-b), R² is 3- to 10-membered heterocycle optionally with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from 3- to 8-membered monocyclic heterocycle and 5- to 10-membered bicyclic heterocycle, each of which is optionally with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), and —CN.

In some embodiments, for the compound or salt of Formula (A), (I′), (I), (II), (II-a), or (II-b), R² is 3- to 8-membered monocyclic heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN; and C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, and —CN. In some embodiments, R² is selected from

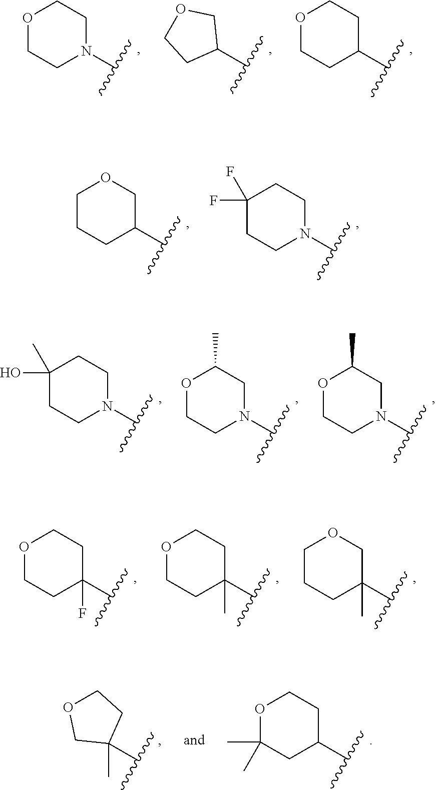

In some embodiments, for the compound or salt of Formula (A), (I′), (I), (II), (II-a), or (II-b), R² is 3- to 8-membered monocyclic heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, ═O, —CN; and C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, and —CN. In some embodiments, R² is selected from

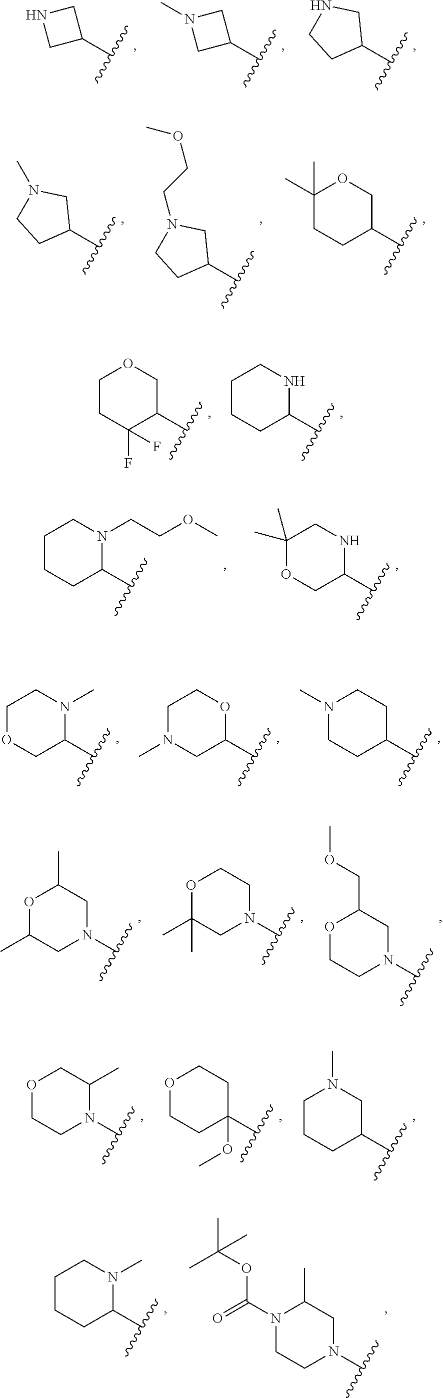

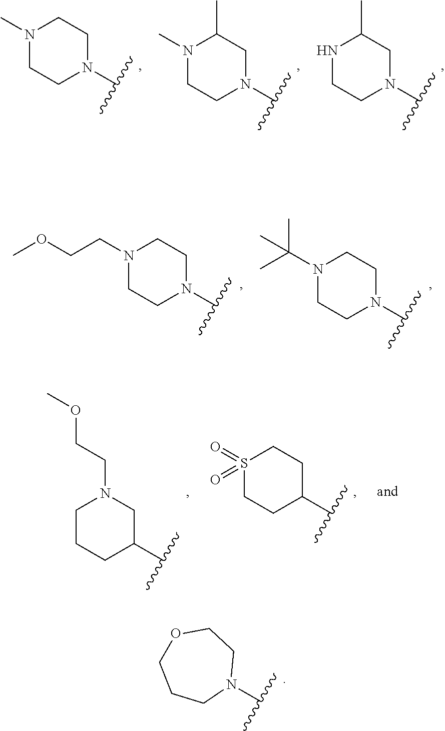

In some embodiments, for the compound or salt of Formula (A), (I′), (I), (II), (II-a), or (II-b), R² is 5- to 10-membered bicyclic heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN; and C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, and —CN. In some embodiments, R² is selected from

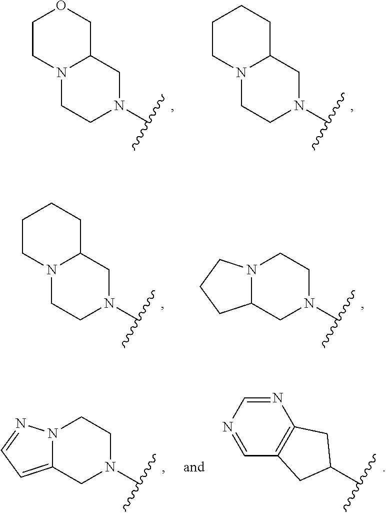 [SMILES: *N1CCN2CCOCC2C1 ; *N1CCN2CCCCC2C1 ; *N1CCN2CCCCC2C1 ; *N1CCN2CCCC2C1 ; *N1CCn2nccc2C1 ; *C1Cc2cncnc2C1]

some embodiments, R² is 5- to 10-membered bridged heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN; and C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, and —CN. In some embodiments, R² is selected from

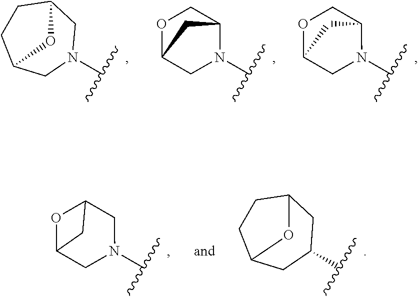

In some embodiments, R² is selected from

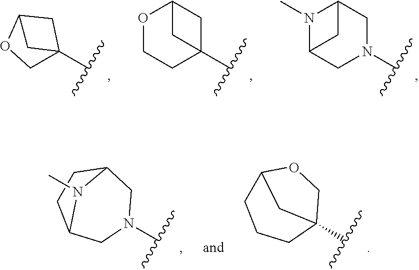

In some embodiments, for the compound or salt of Formula (A), (I′), (I), (II), (II-a), or (II-b), R² is 5- to 10-membered spirocyclic heterocycle optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN; and C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, and —CN. In some embodiments, R² is selected from

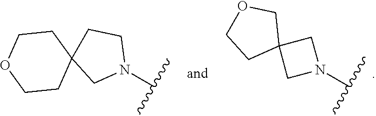 [SMILES: *N1CCC2(CCOCC2)C1 ; *N1CC2(CCOC2)C1]

In some embodiments, R² is selected from

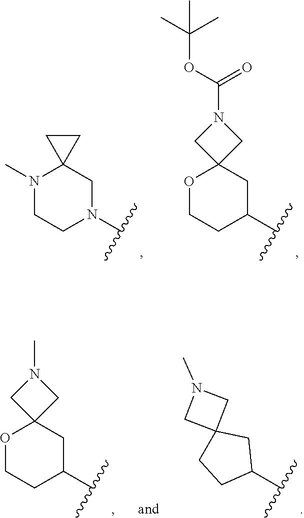

In some embodiments, for the compound or salt of Formula (A), (I′), (I), (II), (II-a), or (II-b), R² is optionally substituted C₃₋₁₀ carbocycle. In some embodiments, R² is optionally substituted C₃₋₁₀ saturated carbocycle. In some embodiments, R² is optionally substituted C₃₋₁₀ unsaturated carbocycle. In some embodiments, R² is selected from C₃₋₄ carbocycle, C₃₋₅ carbocycle, C₃₋₆ carbocycle, C₃₋₇ carbocycle, C₃₋₈ carbocycle, C₃₋₉ carbocycle, and C₃₋₁₀ carbocycle, each of which is optionally substituted with substituents as defined in Formula (I′) or (I). In some embodiments, R² is selected from C₃ carbocycle, C₄ carbocycle, C₅ carbocycle, C₆ carbocycle, C₇ carbocycle, C₈ carbocycle, C₉ carbocycle, and C₁₀ carbocycle, each of which is optionally substituted with substituents as defined in Formula (I) or (I). In some embodiments, R² is selected from C₃₋₈ monocyclic carbocycle and C₅₋₁₀ bicyclic carbocycle, each of which is optionally substituted with substituents as defined in Formula (I). In some embodiments, R² is C₅₋₁₀ bicyclic carbocycle, the C₅₋₁₀ bicyclic carbocycle is selected from C₅₋₁₀ fused carbocycle, C₅₋₁₀ bridged carbocycle, and C₅₋₁₀ spirocyclic carbocycle, each of which is optionally substituted with substituents as defined in Formula (I′) or (I).

In some embodiments, for the compound or salt of Formula (A), (I′), (I), (II), (II-a), or (II-b), R² is selected from C₃₋₁₀ carbocycle optionally substituted with one or more substituents independently selected from halogen, —OR¹⁵, —SR¹⁵, —N(R¹⁵)₂, —NO₂, ═O, —CN; and C₁₋₆ alkyl optionally substituted with one or more substituents independently selected from halogen, —OR¹⁵, —SR¹⁵, —N(R¹⁵)₂, —NO₂, ═O, and —CN. In some embodiments, R² is selected from C₃₋₆ carbocycle optionally substituted with one or more substituents independently selected from halogen, —OR¹⁵, —SR¹⁵, —N(R¹⁵)₂, —NO₂, ═O, —CN; and C₁₋₃ alkyl optionally substituted with one or more substituents independently selected from halogen, —OR¹⁵, —SR¹⁵, —N(R¹⁵)₂, and —CN. In some embodiments, R² is

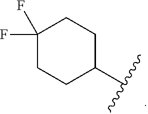 [SMILES: *C1CCC(F)(F)CC1]

In some embodiments, R² is selected from

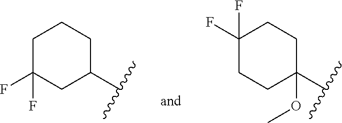 [SMILES: *C1CCCC(F)(F)C1 ; *C1(OC)CCC(F)(F)CC1]

In some embodiments, for the compound or salt of Formula (A), (I′), (I), (II), (II-a), or (II-b), R² is —N(R¹⁵)₂. In some embodiments, R² is —N(R¹⁵)₂ wherein each R¹⁵ is independently selected at each occurrence from:
C₁₋₆ alkyl optionally substituted with one or more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In some embodiments, for the compound or salt of Formula (A), (I′), (I), (II), (II-a), or (II-b), R² is —N(R¹⁵)₂ wherein each R¹⁵ is independently selected at each occurrence from C₁₋₆ alkyl optionally substituted with one or more substituents independently selected from halogen, —O—C₁₋₆ alkyl, —O—C₁₋₆ haloalkyl, —NH₂, —NO₂, ═O, and —CN; and C₃₋₆ carbocycle and 4- to 6-membered heterocycle, the C₃₋₆ carbocycle and 4- to 6-membered heterocycle are each optionally substituted with one or more substituents independently selected from: halogen, —OH, —O—C₁₋₆ alkyl, —O—C₁₋₆ haloalkyl, —NH₂, —NO₂, ═O, and —CN. In some embodiments, R² is selected from

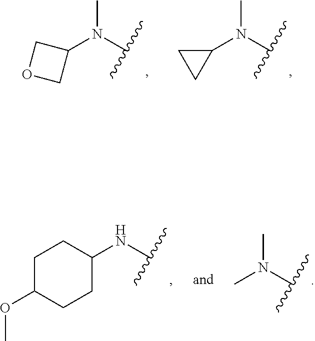

In some embodiments, R² is

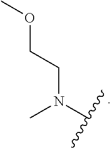 [SMILES: *N(C)CCOC]

In some embodiments, for the compound or salt of Formula (A), (I′), (I), (II), (II-a), or (II-b), R² is —OR¹⁵. In some embodiments, R² is —OR¹⁵ wherein R¹⁵ is independently selected at each occurrence from:
C₁₋₆ alkyl optionally substituted with one or more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In some embodiments, for the compound or salt of Formula (A), (I′), (I), (II), (II-a), or (II-b), R² is —OR¹⁵, wherein R¹⁵ is selected from hydrogen; C₁₋₆ alkyl optionally substituted with one or more substituents independently selected from halogen, —O—C₁₋₆ alkyl, —O—C₁₋₆ haloalkyl, —NH₂, —NO², ═O, and —CN; and C₃₋₆ carbocycle and 4- to 6-membered heterocycle, the C₃₋₆ carbocycle and 4- to 6-membered heterocycle are each optionally substituted with one or more substituents independently selected from: halogen, —OH, —O—C₁₋₆ alkyl, —O—C₁₋₆ haloalkyl, —NH₂, —NO₂, ═O, and —CN. In some embodiments, R² is

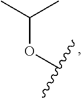 [SMILES: *OC(C)C]

In some aspects, the present disclosure provides a compound of Formula (I′) selected from a compound of Table 1, or pharmaceutically salts thereof.

In some aspects, the present disclosure provides a compound of Formula (I) selected from a compound of Table 1, or pharmaceutically salts thereof.

In some aspects, the present disclosure provides a compound of Formula (II) selected from a compound of Table 1, or pharmaceutically salts thereof.

In some aspects, the present disclosure provides a compound of Formula (II-a) selected from a compound of Table 1, or pharmaceutically salts thereof.

In some aspects, the present disclosure provides a compound of Formula (II-b) selected from a compound of Table 1, or pharmaceutically salts thereof.

In some aspects, the present disclosure provides a compound of Formula (I′) or Formula (I) selected from:

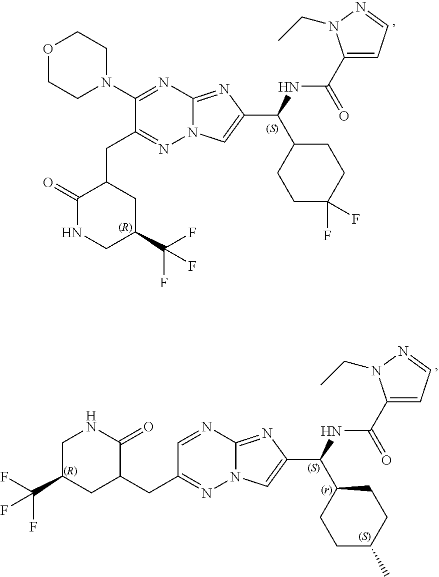

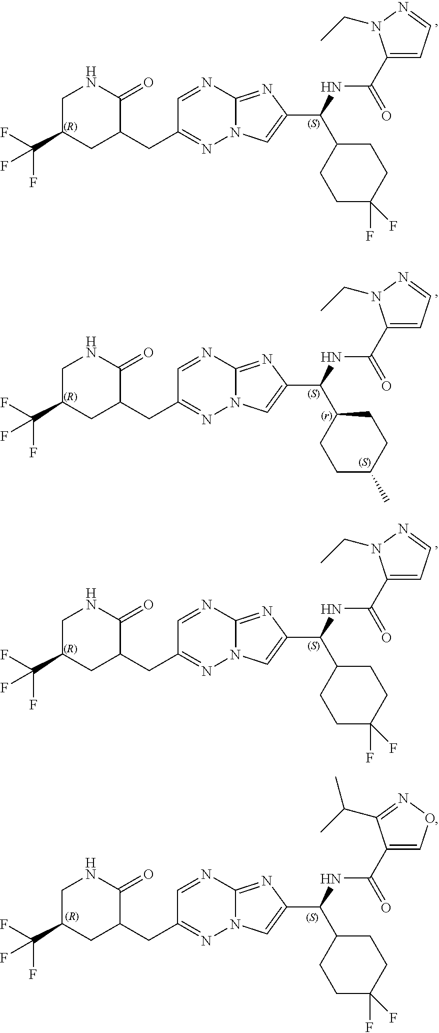

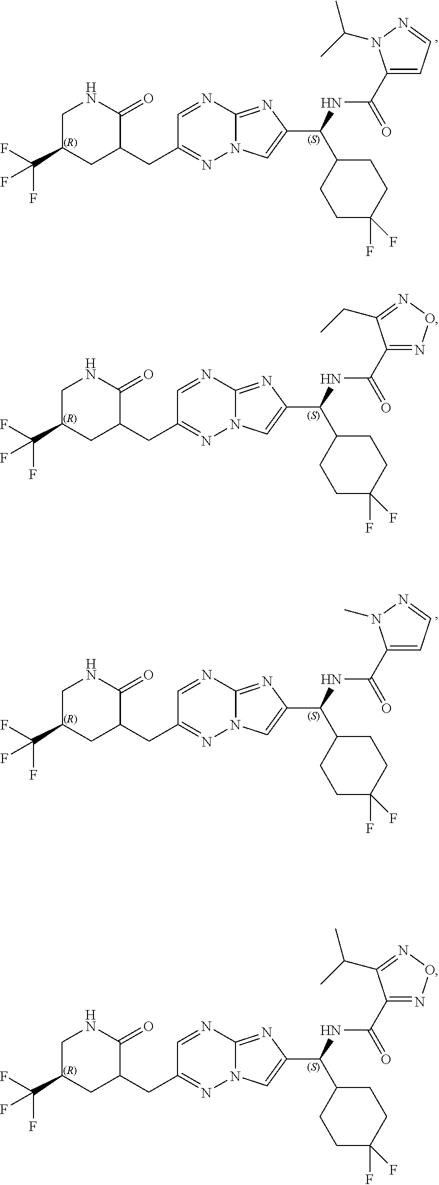

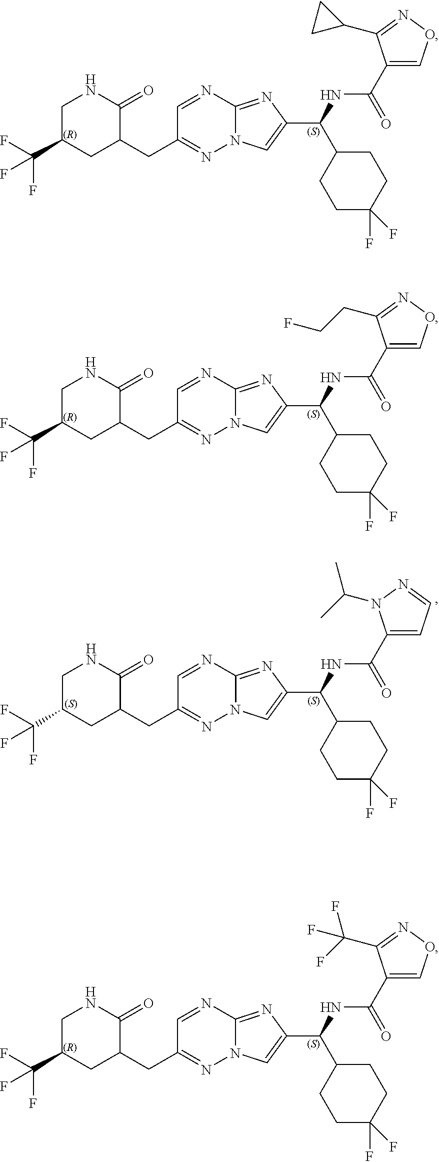

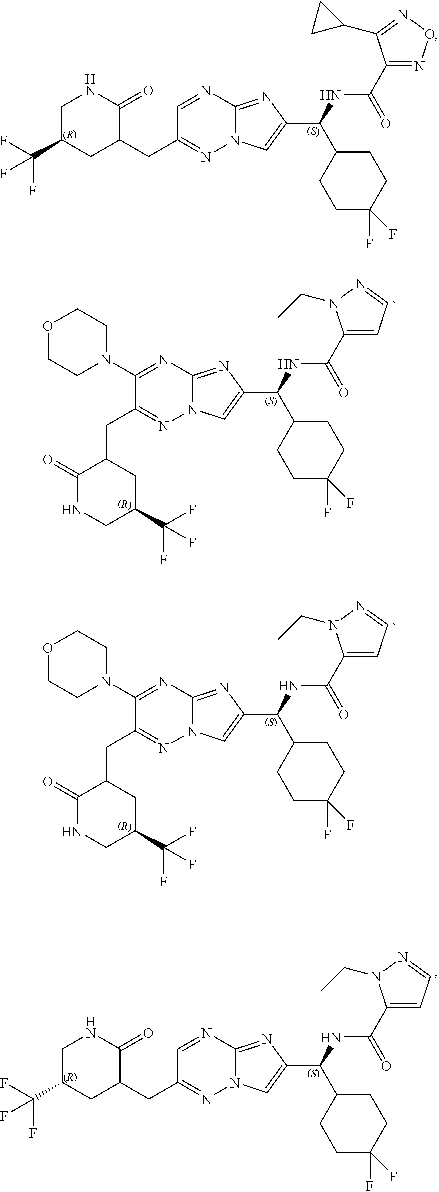

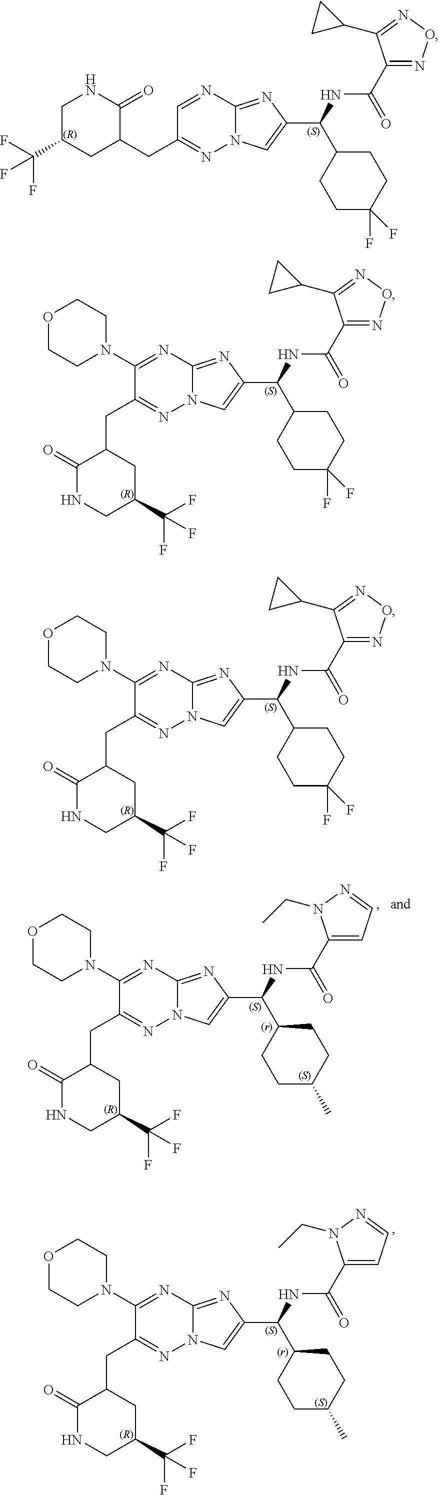

or pharmaceutically acceptable salts thereof.

In certain aspects, the present disclosure provides a compound of formula

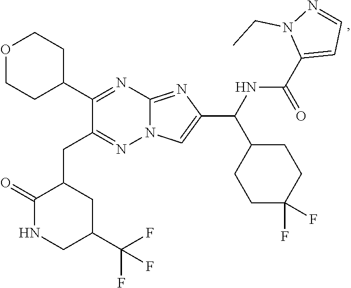 [SMILES: CCn1nccc1C(=O)NC(c1cn2nc(CC3CC(C(F)(F)F)CNC3=O)c(C3CCOCC3)nc2n1)C1CCC(F)(F)CC1]

or a pharmaceutically acceptable salt. In some embodiments, the compound is

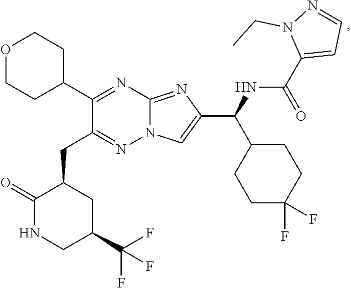 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments the compound is

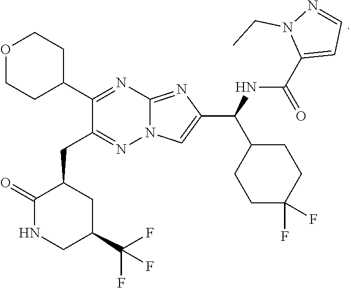 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

In some embodiments, the compound is

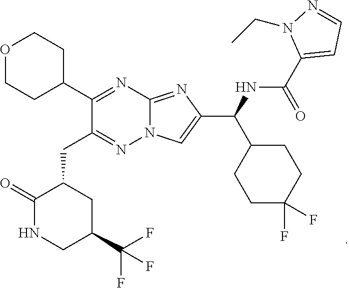 [SMILES: CCn1nccc1C(=O)N[C@H](c1cn2nc(C[C@@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

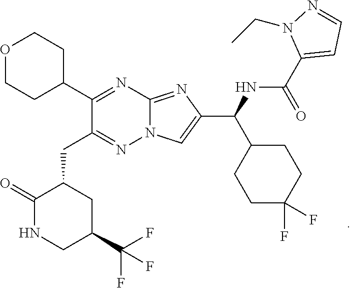 [SMILES: CCn1nccc1C(=O)N[C@H](c1cn2nc(C[C@@H]3C[C@@H](C(F)(F)F)CNC3=O)c(C3CCOCC3)nc2n1)C1CCC(F)(F)CC1]

In some embodiments, the compound is

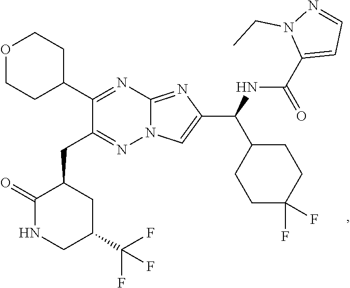 [SMILES: CCn1nccc1C(=O)N[C@H](c1cn2nc(C[C@H]3C[C@H](C(F)(F)F)CNC3=O)c(C3CCOCC3)nc2n1)C1CCC(F)(F)CC1]

or a pharmaceutically acceptable salt thereof. In some embodiments, the compound is

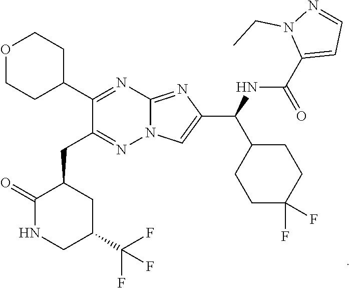 [SMILES: CCn1nccc1C(=O)N[C@H](c1cn2nc(C[C@H]3C[C@H](C(F)(F)F)CNC3=O)c(C3CCOCC3)nc2n1)C1CCC(F)(F)CC1]

In certain aspects, the present disclosure provides a compound of formula

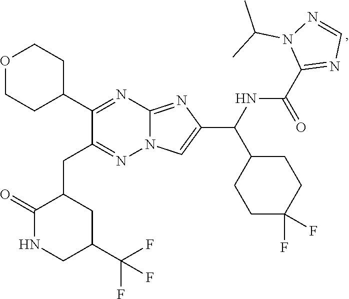 [SMILES: CC(C)n1ncnc1C(=O)NC(c1cn2nc(CC3CC(C(F)(F)F)CNC3=O)c(C3CCOCC3)nc2n1)C1CCC(F)(F)CC1]

r a pharmaceutically acceptable salt thereof. In some embodiments, the compound is

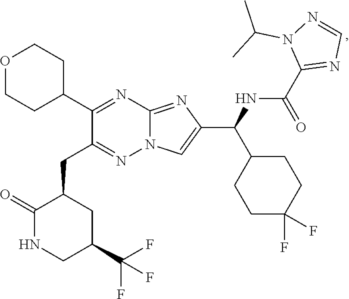 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

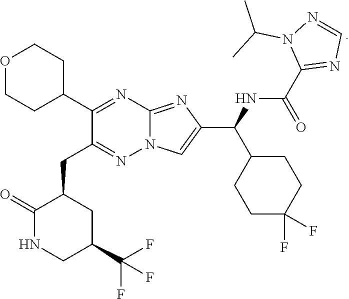 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a compound of formula

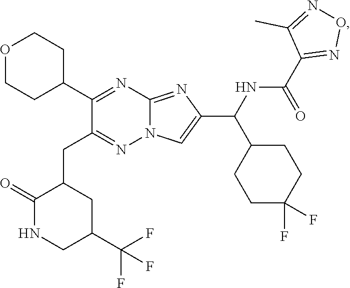 [SMILES: Cc1nonc1C(=O)NC(c1cn2nc(CC3CC(C(F)(F)F)CNC3=O)c(C3CCOCC3)nc2n1)C1CCC(F)(F)CC1]

or a pharmaceutically acceptable salt thereof. In some embodiments, the compound is

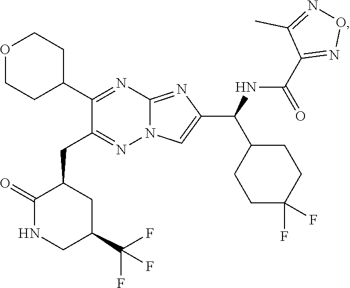 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

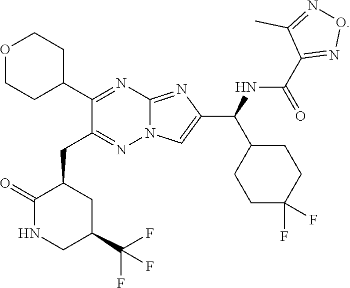 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (A), (I′), (I), (II), (II-a), or (II-b), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (A), (I′), (I), (II), (II-a), or (II-b), may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration. In some aspects, the compounds or salts of Table 1 may in exist as diastereomers, enantiomers, or other stereoisomeric forms. In some embodiments, the compounds or salts of Table 1 are single isomers whose absolute stereochemistry (i.e., arrangement in space of one or more stereocenters) may be unknown.

In certain embodiments, compounds or salts for Formula (A), (I′), (I), (II), (II-a), or (II-b), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975). Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (A), (I′), (I), (II), (II-a), or (II-b), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

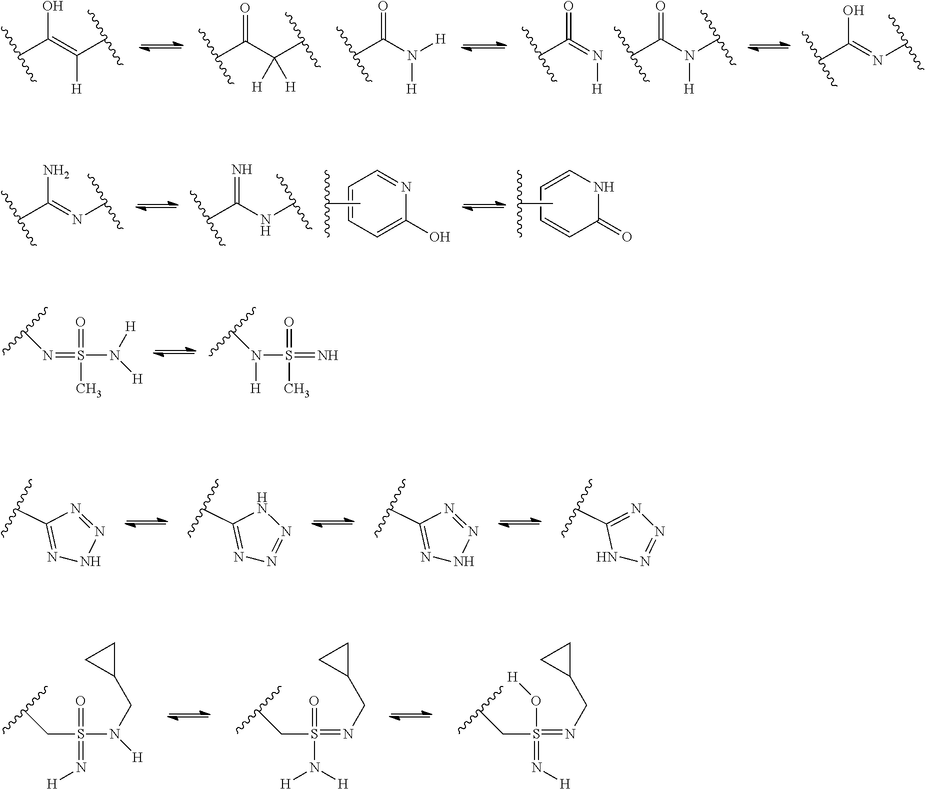

The compounds of Formula (A), (I′), (I), (II), (II-a), or (II-b), can be used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds of Formula (A), (I′), (I), (II), (II-a), or (II-b), have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds of Formula (A), (I′), (I), (II), (II-a), or (II-b), are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of Formula (A), (I′), (I), (II), (II-a), or (II-b), optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (A), (I′), (I), (II), (II-a), or (II-b). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

In certain embodiments, compounds or salts of Formula (A), (I′), (I), (II), (II-a), or (II-b), may be prodrugs. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal.

In some embodiments, the design of a prodrug increases the lipophilicity of the pharmaceutical agent. In some embodiments, the design of a prodrug increases the effective water solubility. See, e.g., Fedorak et al., Am. J Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagentsfor Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (A), (I′), (I), (II), (II-a), or (II-b), and at least one pharmaceutically acceptable excipient. In some embodiments, the pharmaceutical composition comprises a compound or salt of Formula (A), (I′), (I), (II), (II-a), or (II-b), and a pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Pharmaceutical compositions as often further can comprise more than one active compound (e.g., a compound, salt or conjugate and other agents) as necessary for the particular indication being treated. The active compounds can have complementary activities that do not adversely affect each other. Such molecules can be present in combination in amounts that are effective for the purpose intended.

A compound or salt of any one of Formula (A), (I′), (I), (II), (II-a), or (II-b), may be formulated in any suitable pharmaceutical formulation. A pharmaceutical formulation of the present disclosure typically contains an active ingredient (e.g., compound or salt of any one of Formula (A), (I′), (I), (II), (II-a), or (II-b), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

In certain embodiments, a compound or salt of Formula (A), (I′), (I), (II), (II-a), or (II-b), is formulated with a chelating agent or other material capable of binding metal ions, such as ethylene diamine tetra acetic acid (EDTA) and its salts are capable of enhancing the stability of a compound or salt of Formula (A), (I′), (I), (II), (II-a), or (II-b).

Pharmaceutical formulations may be provided in any suitable form, which may depend on the route of administration.

In some embodiments, the disclosure provides a pharmaceutical composition for oral administration containing at least one compound or salt of any one of Formula (A), (I′), (I), (II), (II-a), or (II-b), and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (A), (I′), (I), (II), (II-a), or (II-b), moistened with an inert liquid diluent.

Pharmaceutical compositions may also be prepared from a compound or salt of any one of Formula (A), (I′), (I), (II), (II-a), or (II-b), and one or more pharmaceutically acceptable excipients. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

In some aspects, the present disclosure provides a method of modulating IL-17 A in a subject in need thereof, comprising administering to the subject a compound or salt of Formula (A), (I′), (I), (II), (II-a), or (II-b), or a pharmaceutical composition thereof.

Increased levels of IL-17A have been associated with several conditions including airway inflammation, rheumatoid arthritis (RA), osteoarthritis, bone erosion, intraperitoneal abscesses and adhesions, inflammatory bowel disorder (IBD), allograft rejection, psoriasis, psoriatic arthritis, ankylosing spondylitis, certain types of cancer, angiogenesis, atherosclerosis and multiple sclerosis (MS). Both IL-17A and IL-17R are upregulated in the synovial tissue of RA patients. IL-17A exerts its role in pathogenesis of RA through IL-1-β and TNF-α dependent and independent pathways. IL-17A stimulates secretion of other cytokines and chemokines, e.g., TNF-α, IL-1β, IL-6, IL-8 and Gro-α. IL-17A directly contributes to disease progression in RA. Injection of IL-17A into the mouse knee promotes joint destruction independently of IL-I β activity (Ann Rheum Dis 2000, 59:529-32). Anti-IL-1β antibody has no effect on IL-17A induced inflammation and joint damage (J. Immunol 2001, 167:1004-1013). In a streptococcal cell wall (SCW)-induced murine arthritis model, IL-17A induced inflammatory cell infiltration and proteoglycan depletion in wild-type and IL-1β knockout and TNF-α knockout mice. IL-17A knockout mice are phenotypically normal in the absence of antigenic challenge but have markedly reduced arthritis following type II collagen immunization (J. Immunol 2003, 171:6173-6177). Increased levels of IL-17A-secreting cells have also been observed in the facet joints of patients suffering from ankylosing spondylitis (H Appel et al., Arthritis Res Therap. 2011, 13:R95).

Multiple sclerosis is an autoimmune disease characterized by central nervous system (CNS) inflammation with damage to the myelin sheath surrounding axons. A hallmark of MS is that T cells infiltrate into the CNS. Higher numbers of IL-17A mRNA-expressing blood mono-nuclear cells (MNC) are detected during MS clinical exacerbation compared to remission (Multiple Sclerosis, 5:101-104, 1999). Furthermore, experimental autoimmune encephalomyelitis (“EAE”), a preclinical animal model for MS is significantly suppressed in IL-17A knockout mice.

In certain aspects, the disclosure provides methods of modulating IL-17A in a subject in need thereof, comprising administering to said subject a compound or salt of Formula (A), (I′), (I), (II), (II-a), or (II-b). In certain embodiments, a compound or salt of Formula (A), (I′), (I), (II), (II-a), or (II-b), inhibits the activity of IL-17A in a subject in need thereof.

In certain embodiments, a compound or salt of Formula (A), (I′), (I), (II), (II-a), or (II-b), can be used to treat or prevent a disease or condition that is mediated directly or indirectly by IL-17A. Such diseases include inflammatory diseases and conditions, proliferative diseases (e.g., cancer), autoimmune diseases and other disease described herein. The methods generally involve administering therapeutically effective amounts of compounds disclosed herein or a pharmaceutical composition thereof to the subject. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

In some aspects, the present disclosure provides a method of a method of treating or preventing an inflammatory disease or condition in a subject in need thereof, comprising administering to the subject a compound or salt of Formula (A), (I′), (I), (II), (II-a), or (II-b), or a pharmaceutical composition thereof. In certain embodiments, a compound or salt of Formula (A), (I′), (I), (II), (II-a), or (II-b), is administered to a subject in need thereof to treat an inflammatory disease or condition, e.g., psoriasis.

In certain embodiments, a compound or salt of Formula (A), (I′), (I), (II), (II-a), or (II-b), is used to treat or prevent an inflammatory disease or condition is selected from, plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis. In certain embodiments, a compound or salt of Formula (A), (I′), (I), (II), (II-a), or (II-b), is used to treat or prevent psoriasis. In certain embodiments, a compound or salt of Formula (A), (I′), (I), (II), (II-a), or (II-b), is used for the treatment or prevention of a condition including, but not limited to, airway inflammation, ankylosing spondylitis, asthma, RA (including juvenile RA), as well as other inflammatory disorders, conditions, or diseases.

In certain embodiments, the present disclosure provides a method of treating psoriasis comprising administering to a subject in need thereof a compound or salt of Formula (A), (I′), (I), (II), (II-a), or (II-b).

In certain embodiments, the present disclosure provides a method of treating psoriatic arthritis comprising administering to a subject in need thereof a compound or salt of Formula (A), (I′), (I), (II), (II-a), or (II-b).

In certain embodiments, the present disclosure provides a method of treating ankylosing spondylitis comprising administering to a subject in need thereof a compound or salt of Formula (A), (I′), (I), (II), (II-a), or (II-b).

In certain aspects, the present disclosure provides a method of treating an inflammatory disease or an inflammatory condition selected from the group consisting of ankylosing spondylitis, aspsoriatic arthritis, erythrodermic psoriasis, guttate psoriasis, hidradenitis suppurutiva, inverse psoriasis, non-infectious uveitis, palmoplantar psoriasis, plaque psoriasis, pustular psoriasis, rheumatoid arthritis, and spondyloarthritis, wherein the method comprises administering to a subject in need thereof a compound of the formula:

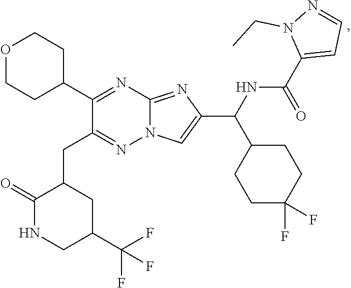 [SMILES: CCn1nccc1C(=O)NC(c1cn2nc(CC3CC(C(F)(F)F)CNC3=O)c(C3CCOCC3)nc2n1)C1CCC(F)(F)CC1]

or a pharmaceutically acceptable salt thereof. In some embodiments, the compound is

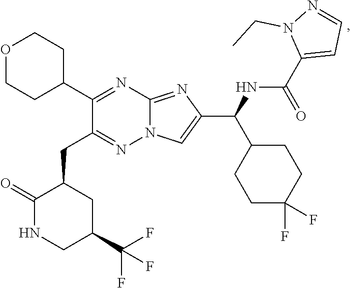 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

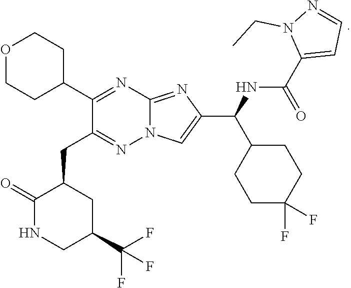 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriasis comprising administering to a subject in need thereof a compound of the formula:

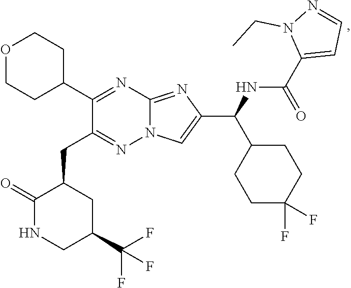 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

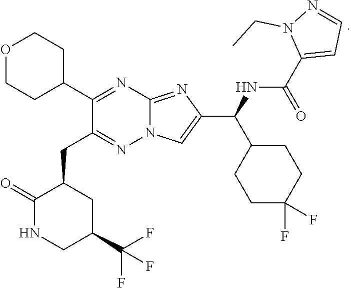 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriatic arthritis comprising administering to a subject in need thereof a compound of the formula:

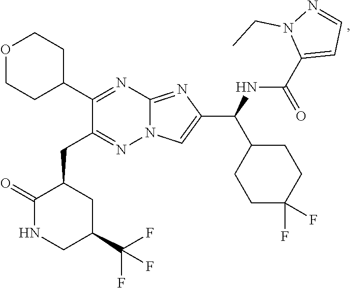 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

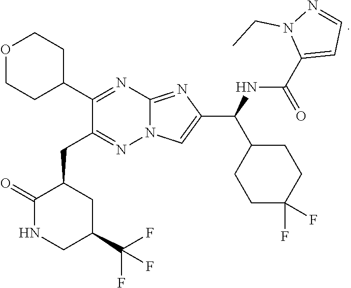 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating ankylosing spondylitis comprising administering to a subject in need thereof a compound of the formula:

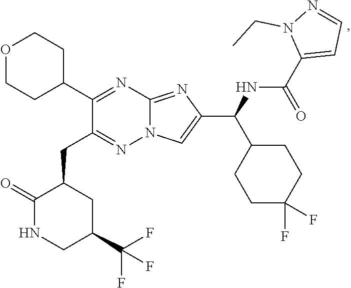 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

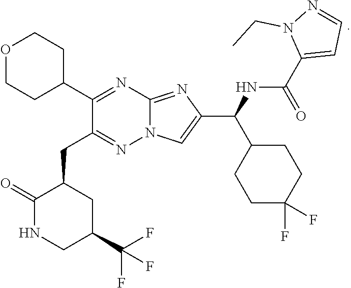 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating an inflammatory disease or an inflammatory condition selected from the group consisting of ankylosing spondylitis, aspsoriatic arthritis, erythrodermic psoriasis, guttate psoriasis, hidradenitis suppurutiva, inverse psoriasis, non-infectious uveitis, palmoplantar psoriasis, plaque psoriasis, pustular psoriasis, rheumatoid arthritis, and spondyloarthritis, wherein the method comprises administering to a subject in need thereof a compound of the formula:

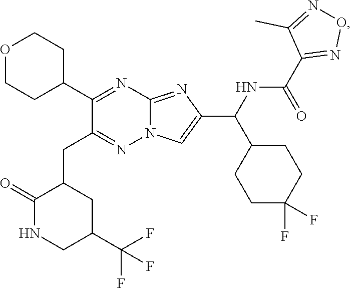 [SMILES: Cc1nonc1C(=O)NC(c1cn2nc(CC3CC(C(F)(F)F)CNC3=O)c(C3CCOCC3)nc2n1)C1CCC(F)(F)CC1]

or a pharmaceutically acceptable salt thereof. In some embodiments, the compounds is

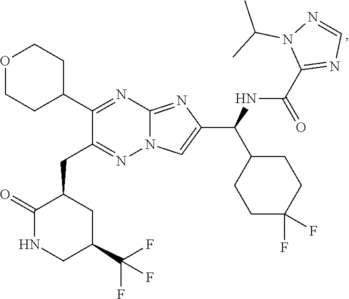 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

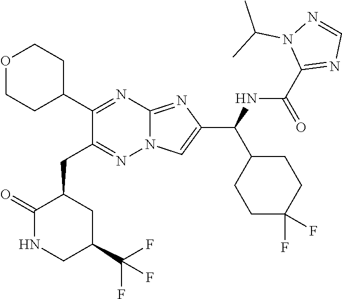 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriasis comprising administering to a subject in need thereof a compound of the formula:

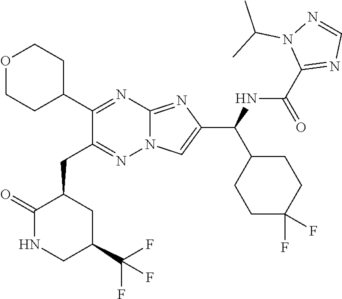 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

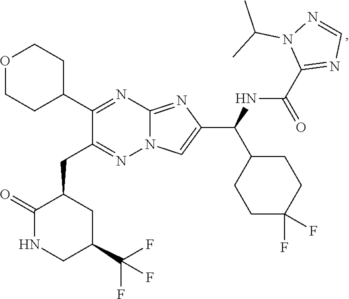 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriatic arthritis comprising administering to a subject in need thereof a compound of the formula:

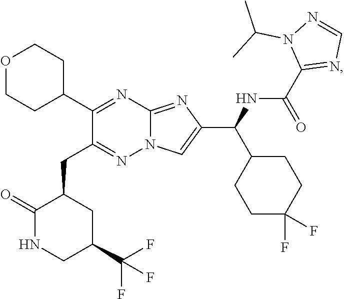 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

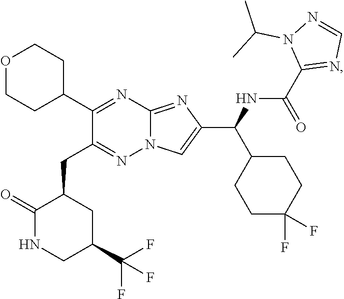 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating ankylosing spondylitis comprising administering to a subject in need thereof a compound of the formula:

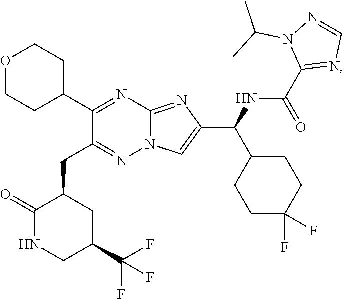 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

pr a pharmaceutically acceptable salt. In some embodiments, the compounds is

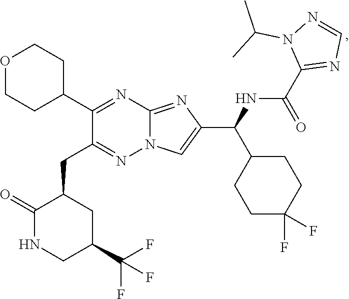 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating an inflammatory disease or an inflammatory condition selected from the group consisting of ankylosing spondylitis, aspsoriatic arthritis, erythrodermic psoriasis, guttate psoriasis, hidradenitis suppurutiva, inverse psoriasis, non-infectious uveitis, palmoplantar psoriasis, plaque psoriasis, pustular psoriasis, rheumatoid arthritis, and spondyloarthritis, wherein the method comprises administering to a subject in need thereof a compound of the formula:

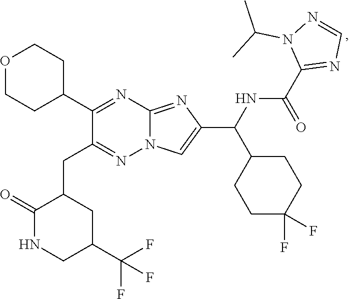 [SMILES: CC(C)n1ncnc1C(=O)NC(c1cn2nc(CC3CC(C(F)(F)F)CNC3=O)c(C3CCOCC3)nc2n1)C1CCC(F)(F)CC1]

or a pharmaceutically acceptable salt thereof. In some embodiments, the compounds is

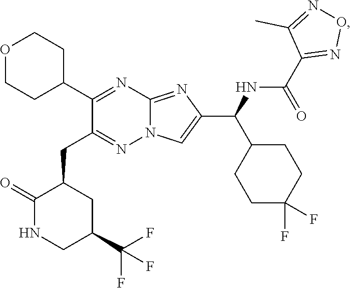 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt thereof. In some embodiments, the compound is

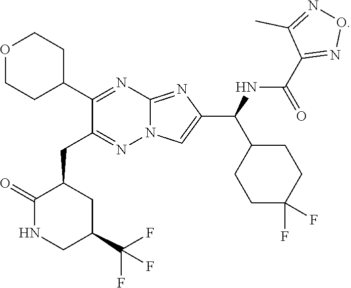 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriasis comprising administering to a subject in need thereof a compound of the formula:

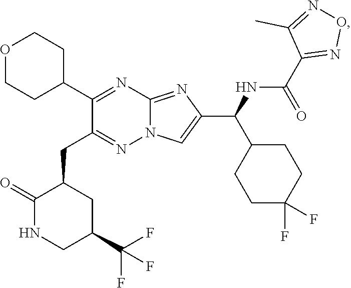 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

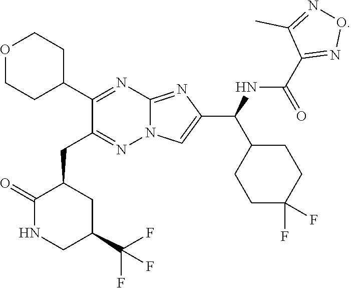 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating psoriatic arthritis comprising administering to a subject in need thereof a compound of the formula:

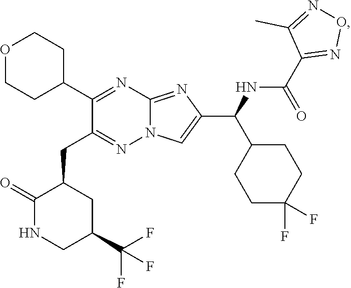 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is

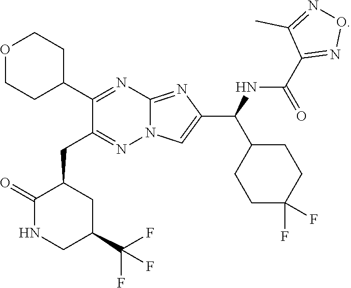 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

In certain aspects, the present disclosure provides a method of treating ankylosing spondylitis comprising administering to a subject in need thereof a compound of the formula:

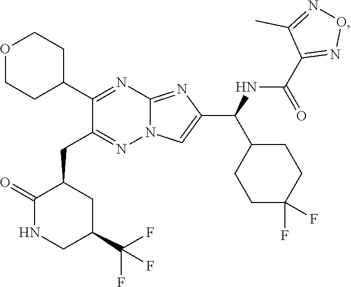 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

or a pharmaceutically acceptable salt. In some embodiments, the compounds is F

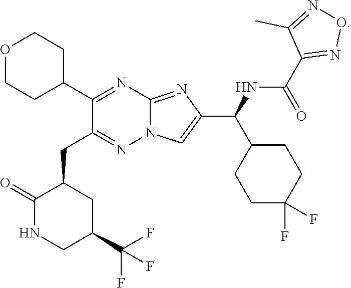 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]

### EXAMPLES

The invention now being generally described, it will be more readily understood by reference to the following examples which are included merely for purposes of illustration of certain aspects and embodiments of the present invention and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-313 show exemplary procedures for the preparation of the claimed IL-17A modulators. Example 314 provides IL-17 A/A and A/F IC₅₀ inhibition data and Example 315 provides mouse oral bioavailability data.

### Example 1: Preparation of Benzyl ((S)-3-bromo-1-((1R,4S)-4-methylcyclohexyl)-2-oxopropyl)carbamate

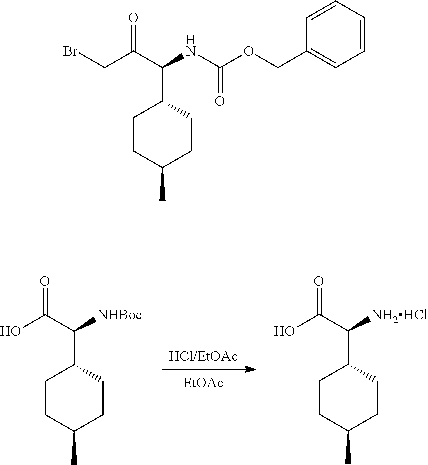

To a solution of (S)-2-((tert-butoxycarbonyl)amino)-2-((1R,4S)-4-methylcyclohexyl)acetic acid (250 g, 921 mmol, 1.00 eq) in EtOAc (500 mL) was added a solution of HCl in EtOAc (4 M, 2.5 L, 10.8 eq). The reaction mixture was stirred at a temperature maintained between 0 and 10° C. for 2 h. The reaction mixture was then concentrated under reduced pressure to afford (S)-2-amino-2-((1R,4S)-4-methylcyclohexyl)acetic acid (200 g, crude, HCl salt) as a white solid.

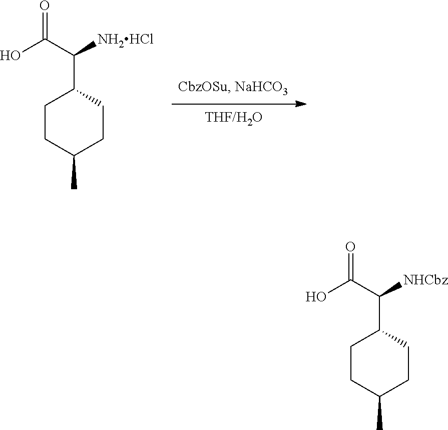

To a solution of (S)-2-amino-2-((1R,4S)-4-methylcyclohexyl)acetic acid (193 g, 934 mmol, 1.00 eq) in THF (800 mL) and water (800 mL) was added potassium carbonate (387 g, 2.80 mol, 3.00 eq) followed by N-(Benzyloxycarbonyloxy)succinimide (233 g, 934 mmol, 1.00 eq). The reaction mixture was stirred at a temperature maintained between 10 and 20° C. for 12 h. The reaction mixture was extracted twice with EtOAc. The combined organic layers were washed with brine, dried over anhydrous sodium sulfate, filtered and concentrated under reduced pressure to give a residue, which was triturated with isopropanol at 25° C. for 30 min to afford (S)-2-(((benzyloxy)carbonyl)amino)-2-((1R,4S)-4-methylcyclohexyl)acetic acid (200 g, 633 mmol, 67.8% yield) as a white solid. LCMS [M+Na]⁺=328.2 m/z.

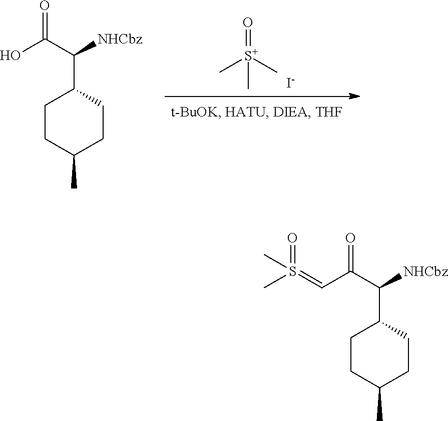

To a solution of trimethylsulfoxonium iodide (648 g, 2.95 mol, 3.00 eq) in THF (1500 mL) was added potassium tert-butoxide (330 g, 2.95 mol, 3.00 eq). The reaction mixture was stirred at 80° C. for 2 h (Reaction 1). In a separate flask, to a solution of (S)-2-(((benzyloxy)carbonyl)amino)-2-((1R,4S)-4-methylcyclohexyl)acetic acid (300 g, 982 mmol, 1.00 eq) in THF (1500 mL) was added N,N-diisopropylethylamine (165 g, 1.28 mol, 222 mL, 1.30 eq) followed by 1-[bis(dimethylamino)methylene]-1H-1,2,3-triazolo[4,5-b]pyridinium 3-oxide hexafluorophosphate (HATU, 448 g, 1.18 mol, 1.20 eq). The reaction mixture was stirred at 25° C. for 2 h before adding the trimethylsulfoxonium solution to the HATU adduct slowly at 25° C. The resulting mixture was stirred at 25° C. for 2 h. The reaction mixture was quenched by the addition of water and then diluted with EtOAc. An extraction was performed twice with EtOAc. The combined organic layers were washed with brine, dried over anhydrous sodium sulfate, filtered, and concentrated under reduced pressure to give a residue, which was purified by column chromatography (SiO₂, petroleum ether/EtOAc) to afford benzyl ((S)-3-(dimethyl(oxo)-l⁶-sulfaneylidene)-1-((1R,4S)-4-methylcyclohexyl)-2-oxopropyl)carbamate (400 g, crude) as an off-white solid. LCMS [M+H]⁺=380.1 m/z.

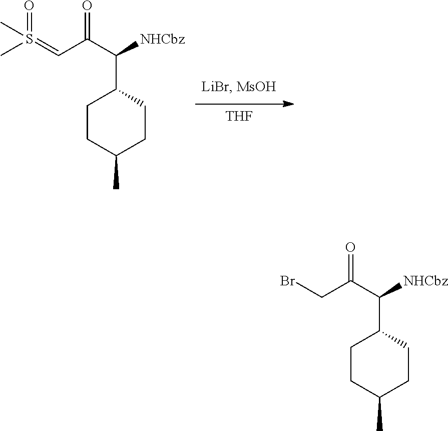 [SMILES: CS(C)(=O)=CC(=O)[C@@H](NC(=O)OCc1ccccc1)[C@H]1CC[C@H](C)CC1 ; C[C@H]1CC[C@H]([C@H](NC(=O)OCc2ccccc2)C(=O)CBr)CC1 ; "THF"]

To a solution of benzyl ((S)-3-(dimethyl(oxo)-l⁶-sulfaneylidene)-1-((1R,4S)-4-methylcyclohexyl)-2-oxopropyl)carbamate (150 g, 395 mmol, 1.00 eq) in THF (1500 mL) was added lithium bromide (34.3 g, 395 mmol, 1.00 eq) followed by methanesulfonic acid (38.0 g, 395 mmol, 28.1 mL, 1.00 eq). The reaction mixture was stirred at 45° C. for 12 h. The reaction mixture was quenched by the addition of water, and then extracted twice with EtOAc. The combined organic layers were washed with brine, dried over anhydrous sodium sulfate, filtered, and concentrated under reduced pressure to give a residue, which was purified by prep-HPLC (column: Phenomenex luna C18 250*100 mm*10 mm; mobile phase: [water (FA)—ACN]; B %: 60%-80%) to afford benzyl ((S)-3-bromo-1-((1R,4S)-4-methylcyclohexyl)-2-oxopropyl)carbamate (18.2 g, 47.8 mmol, 12.1% yield) as a white solid. ¹H NMR (400 MHz, CDCl₃) d 7.31-7.37 (m, 5H), 5.31-5.33 (m, 1H), 5.09 (s, 2H), 4.58-4.60 (m, 1H), 3.99-4.10 (m, 2H), 1.65-1.80 (m, 4H), 1.42-1.48 (m, 1H), 1.25-1.40 (m, 2H), 0.91-1.10 (m, 3H), 0.86-0.88 (m, 3H). LCMS [M+H]⁺=382.1 m/z.

### Example 2: Preparation of Benzyl (S)-(3-bromo-1-(4,4-difluorocyclohexyl)-2-oxopropyl)carbamate

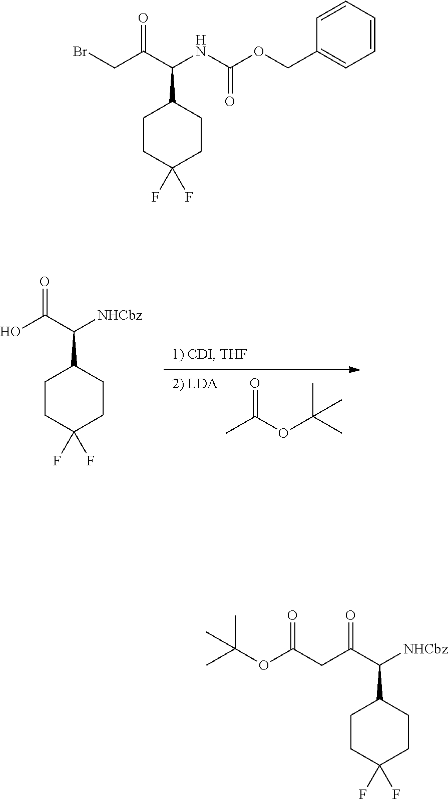

A solution of (S)-2-(((benzyloxy)carbonyl)amino)-2-(4,4-difluorocyclohexyl)acetic acid (30.0 g, 91.6 mmol, 1.00 eq) in THF (320 mL) and 1,1′-carbonyldiimidazole (16.3 g, 100 mmol, 1.10 eq) was stirred at 0° C. for 2 h. Meanwhile, a solution of tert-butyl acetate (31.9 g, 274 mmol, 36.8 mL, 3.00 eq) in THF (200 mL) was added a solution of lithium diisopropylamide (2 M, 160 mL, 3.50 eq), and the mixture was stirred at −70° C. for 1 h. The two reaction mixtures were combined and stirred at −70° C. for 1 h. The resulting mixture was diluted with a saturated aqueous solution of ammonium chloride, extracted with EtOAc, washed with brine, dried over anhydrous sodium sulfate, filtered, and concentrated under reduced pressure to give a residue, which was purified by column chromatography (SiO₂, petroleum ether/EtOAc) to afford tert-butyl (S)-4-(((benzyloxy)carbonyl)amino)-4-(4,4-difluorocyclohexyl)-3-oxobutanoate (30.0 g, 70.5 mmol, 76.9% yield) as a yellow oil. LCMS [M+Na]⁺=448.1 m/z.

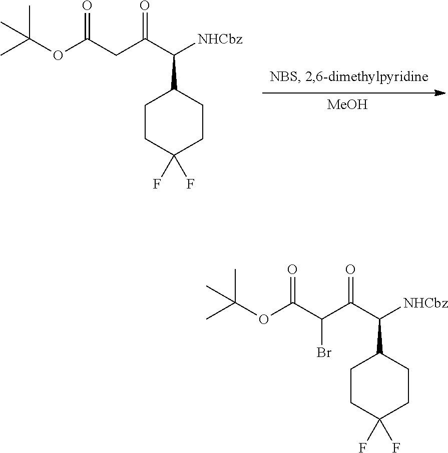 [SMILES: "MeOH" ; CC(C)(C)OC(=O)CC(=O)[C@@H](NC(=O)OCc1ccccc1)C1CCC(F)(F)CC1 ; CC(C)(C)OC(=O)C(Br)C(=O)[C@@H](NC(=O)OCc1ccccc1)C1CCC(F)(F)CC1]

To a solution of tert-butyl (S)-4-(((benzyloxy)carbonyl)amino)-4-(4,4-difluorocyclohexyl)-3-oxobutanoate (30.0 g, 70.5 mmol, 1.00 eq) in MeOH (300 mL) was added N-bromosuccinimide (10.0 g, 56.4 mmol, 0.800 eq) and 2,6-dimethylpyridine (610 mg, 5.64 mmol, 0.08 eq) at 0° C. The mixture was stirred at RT for 2 h. The reaction was diluted with a saturated aqueous solution of sodium hydrogen carbonate, extracted with EtOAc, dried over anhydrous sodium sulfate, filtered, and concentrated under reduced pressure to afford tert-butyl (4S)-4-(((benzyloxy)carbonyl)amino)-2-bromo-4-(4,4-difluorocyclohexyl)-3-oxobutanoate (20.0 g, 39.6 mmol, 56.2% yield) as a yellow solid.

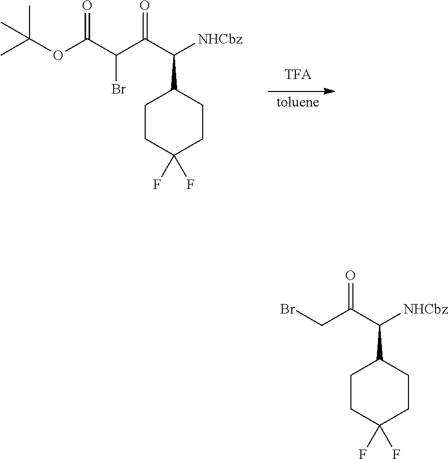 [SMILES: CC(C)(C)OC(=O)C(Br)C(=O)[C@@H](NC(=O)OCc1ccccc1)C1CCC(F)(F)CC1 ; O=C(N[C@H](C(=O)CBr)C1CCC(F)(F)CC1)OCc1ccccc1]

To a solution of tert-butyl (4S)-4-(((benzyloxy)carbonyl)amino)-2-bromo-4-(4,4-difluorocyclohexyl)-3-oxobutanoate (20.0 g, 39.6 mmol, 1.00 eq) in toluene (300 mL) was added trifluoroacetic acid (27.1 g, 237 mmol, 17.6 mL, 6.00 eq). The reaction mixture was stirred at 75° C. for 2 h. The reaction mixture was diluted with a saturated aqueous solution of sodium hydrogen carbonate, and extracted with EtOAc, dried over anhydrous sodium sulfate, filtered, and concentrated under reduced pressure to give a residue, which was purified by prep-HPLC (basic condition; column: Kromasil Eternity XT (250 mm×80 mm, 10 mm); mobile phase: [water (NH₄HCO₃)—ACN]; B %: 45%-70%) to afford benzyl (S)-(3-bromo-1-(4,4-difluorocyclohexyl)-2-oxopropyl)carbamate. The title compound was isolated as the first eluting, single stereoisomer by chiral SFC purification (column: Daicel Chiralcel OJ (250 mm×50 mm, 10 mm); mobile phase: [Neu-MeOH]; B %: 20%); (12.0 g, 26.4 mmol, 66.7% yield) as a white solid. ¹H NMR (400 MHz, CDCl₃) d 7.04-7.34 (m, 5H), 5.34 (d, J=8.8 Hz, 1H), 5.12 (s, 2H), 4.67 (dd, J=5.2 Hz, J=8.8 Hz, 1H), 4.03 (d, J=7.2 Hz, 2H), 2.15-2.12 (m, 2H), 1.97-1.94 (m, 1H), 1.81-1.35 (m, 6H). LCMS [M+H]⁺=404.0 m/z.

### Example 3: Preparation of Benzyl (S)-4-bromo-1,1-dicyclopropyl-3-oxobutan-2-yl)carbamate

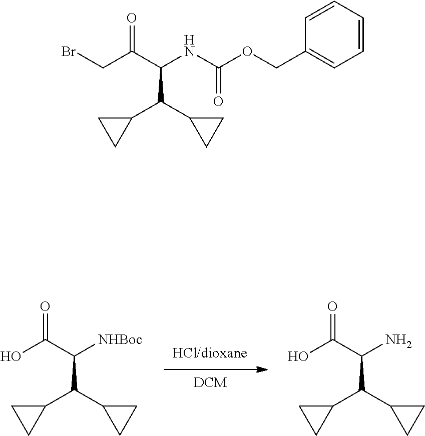

A solution of (S)-2-((tert-butoxycarbonyl)amino)-3,3-dicyclopropylpropanoic acid (15.0 g, 55.6 mmol, 1.00 eq) in dichloromethane (150 mL) and hydrochloric acid in dioxane (4 M, 25.0 mL, 1.80 eq) was stirred at 20° C. for 2 h. The reaction mixture was concentrated under reduced pressure to afford (S)-2-amino-3,3-dicyclopropylpropanoic acid (11.0 g, 53.4 mmol, 96.0% yield, HCl salt) as a white solid. LCMS [M+H]⁺=170.1 m/z.

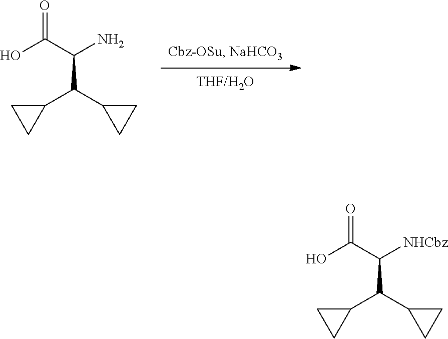 [SMILES: N[C@H](C(=O)O)C(C1CC1)C1CC1 ; O=C(N[C@H](C(=O)O)C(C1CC1)C1CC1)OCc1ccccc1]

To a solution of (S)-2-amino-3,3-dicyclopropylpropanoic acid (11.0 g, 53.4 mmol, 1.00 eq, HCl salt) in tetrahydrofuran (100 mL) and water (50.0 mL) was added benzyl (2,5-dioxopyrrolidin-1-yl) carbonate (26.7 g, 106 mmol, 2.00 eq) and sodium hydrogen carbonate (13.5 g, 160 mmol, 6.24 mL, 3.00 eq). The reaction mixture was stirred at room temperature for 2 h. The reaction mixture was diluted with a 1M aqueous solution of hydrochloric acid to pH=4 and extracted with ethyl acetate, dried over anhydrous sodium sulfate, filtered, and concentrated under reduced pressure to give a residue which was purified by column chromatography (SiO₂, petroleum ether/ethyl acetate) to afford (S)-2-(((benzyloxy)carbonyl)amino)-3,3-dicyclopropylpropanoic acid (10.0 g, 32.9 mmol, 61.6% yield) as a white solid. ¹H NMR (400 MHz, CDCl₃) δ 7.41-7.30 (m, 5H), 5.57 (d, J=9.2 Hz, 1H), 5.14 (s, 2H), 4.73-4.47 (m, 1H), 0.88-0.67 (m, 3H), 0.59-0.35 (m, 4H), 0.32-0.13 (m, 4H). LCMS [M+H]⁺=304.2 m/z.

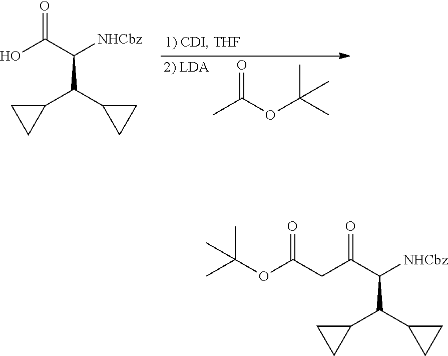

A solution of (S)-2-(((benzyloxy)carbonyl)amino)-3,3-dicyclopropylpropanoic acid (20.0 g, 65.9 mmol, 1.00 eq) in tetrahydrofuran (250 mL) and 1,1′-carbonyldiimidazole (11.8 g, 72.5 mmol, 1.10 eq) was stirred at 0° C. for 2 h. Meanwhile, to a solution of tert-butyl acetate (23.0 g, 198 mmol, 26.5 mL, 3.00 eq) in tetrahydrofuran (250 mL) was added a solution of lithium diisopropylamide (2 M, 98.9 mL, 3.00 eq) and the reaction mixture was stirred at −78° C. for 2 h. The two reaction mixtures were mixed and stirred at −78° C. for 2 h. The reaction mixture was diluted with a saturated aqueous solution of ammonium chloride, extracted with ethyl acetate, dried over anhydrous sodium sulfate, filtered, and concentrated under reduced pressure to give a residue which was purified by column chromatography (SiO₂, petroleum ether/ethyl acetate) to afford tert-butyl (S)-4-(((benzyloxy)carbonyl)amino)-5,5-dicyclopropyl-3-oxopentanoate (15.0 g, 37.3 mmol, 56.6% yield) as a yellow oil. LCMS [M+Na]⁺=424.3 m/z.

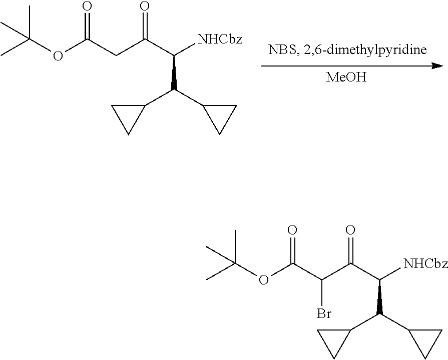 [SMILES: "MeOH" ; CC(C)(C)OC(=O)CC(=O)[C@@H](NC(=O)OCc1ccccc1)C(C1CC1)C1CC1 ; CC(C)(C)OC(=O)C(Br)C(=O)[C@@H](NC(=O)OCc1ccccc1)C(C1CC1)C1CC1]

To a solution of tert-butyl (S)-4-(((benzyloxy)carbonyl)amino)-5,5-dicyclopropyl-3-oxopentanoate (15.0 g, 37.3 mmol, 1.00 eq) in methanol (150 mL) was added N-bromosuccinimide (5.32 g, 29.8 mmol, 0.800 eq) and 2,6-dimethylpyridine (321 mg, 2.99 mmol, 348 μL, 0.0800 eq) at 0° C. The reaction mixture was stirred at 15° C. for 2 h. The reaction mixture was concentrated under reduced pressure to afford tert-butyl (4S)-4-(((benzyloxy)carbonyl)amino)-2-bromo-5,5-dicyclopropyl-3-oxopentanoate (15.0 g, 31.2 mmol, 83.5% yield) as a yellow solid. LCMS [M+H]⁺=480.2 m/z.

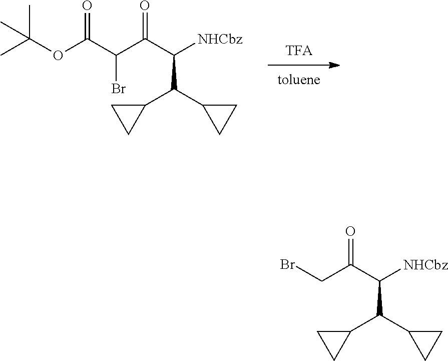 [SMILES: CC(C)(C)OC(=O)C(Br)C(=O)[C@@H](NC(=O)OCc1ccccc1)C(C1CC1)C1CC1 ; O=C(N[C@H](C(=O)CBr)C(C1CC1)C1CC1)OCc1ccccc1]

To a solution of tert-butyl (4S)-4-(((benzyloxy)carbonyl)amino)-2-bromo-5,5-dicyclopropyl-3-oxopentanoate (15.0 g, 31.2 mmol, 1.00 eq) in toluene (150 mL) was added trifluoroacetic acid (17.8 g, 156 mmol, 11.5 mL, 5.00 eq). The reaction mixture was stirred at 80° C. for 2 h. The residue was diluted with a saturated aqueous solution of sodium hydrogen carbonate to pH=7 and extracted with ethyl acetate, dried over anhydrous sodium sulfate, filtered, and concentrated under reduced pressure to give a residue which was purified by prep-HPLC (neutral condition; column: Kromasil Eternity XT (250 mm×80 mm, 10 μm); mobile phase: [water (NH₄HCO₃)—acetonitrile]; B %: 45%-75%) to afford benzyl (S)-(4-bromo-1,1-dicyclopropyl-3-oxobutan-2-yl)carbamate. The title compound was isolated as the first eluting, single stereoisomer by chiral SFC purification (column: Daicel Chiralcel OJ (250 mm×50 mm, 10 μm); mobile phase: [Neu-MeOH]; B %: 20%-20%, 4.3 min); (6.00 g, 15.7 mmol, 50.3% yield) and was obtained as a white solid. ¹H NMR (400 MHz, CDCl₃) δ 7.43-7.31 (m, 5H), 5.65-5.55 (d, J=8.0 Hz, 1H), 5.12 (s, 2H), 4.88-4.78 (m, 1H), 4.26-4.10 (m, 2H), 0.79-0.63 (m, 3H), 0.59-0.41 (m, 4H), 0.30-0.14 (m, 4H). LCMS [M+H]⁺=380.1 m/z.

### Example 4: Preparation of Benzyl (S)-3-bromo-1-cycloheptyl-2-oxopropyl)carbamate

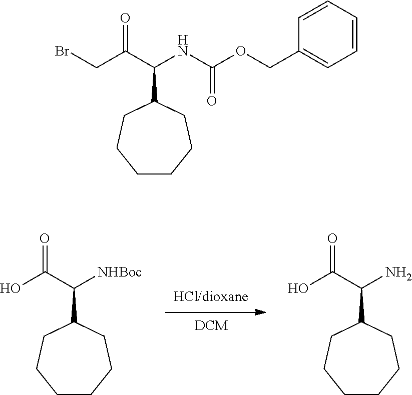

To a solution of (S)-2-((tert-butoxycarbonyl)amino)-2-cycloheptylacetic acid (45.0 g, 147 mmol, 1.00 eq) in dichloromethane (400 mL) was added hydrochloric acid in dioxane (4.00 M, 368 mL, 10.0 eq) at 0° C. The mixture was stirred at room temperature for 3 h. The reaction mixture was concentrated under reduced pressure to afford (S)-2-amino-2-cycloheptylacetic acid (30.0 g, 144 mmol, 97.9% yield, HCl salt) as a white solid. LCMS [M+H]⁺=172.2 m/z.

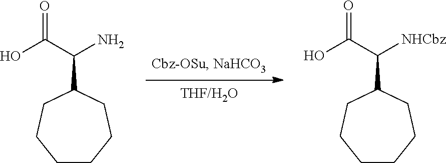 [SMILES: N[C@H](C(=O)O)C1CCCCCC1 ; "THF/H2O" ; O=C(N[C@H](C(=O)O)C1CCCCCC1)OCc1ccccc1]

To a solution of (S)-2-amino-2-cycloheptylacetic acid (30.0 g, 144 mmol, 1.00 eq, HCl salt) in tetrahydrofuran (300 mL) and water (100 mL) was added benzyl (2,5-dioxopyrrolidin-1-yl) carbonate (71.9 g, 288 mmol, 2.00 eq) and sodium hydrogen carbonate (36.4 g, 433 mmol, 16.8 mL, 3.00 eq). The reaction mixture was stirred at room temperature for 2 h. The reaction mixture was diluted with a 1M aqueous solution of hydrochloric acid to adjust pH=2, extracted with dichloromethane. The combined organic layers were dried over anhydrous sodium sulfate, filtered, and concentrated under reduced pressure to give a residue which was purified by column chromatography (SiO₂, petroleum ether/ethyl acetate) to afford (S)-2-(((benzyloxy)carbonyl)amino)-2-cycloheptylacetic acid (43.0 g, 140 mmol, 97.4% yield) as a white solid. ¹H NMR (400 MHz, CDCl₃) δ 7.42-7.31 (m, 5H), 5.27 (d, J=9.2 Hz, 1H), 5.13 (s, 2H), 4.40-4.37 (dd, J1=4.0, J2=8.8 Hz, 1H), 2.19-2.05 (d, J=3.5 Hz, 1H), 1.80-1.30 (m, 12H). LCMS [M+H]⁺=306.1 m/z.

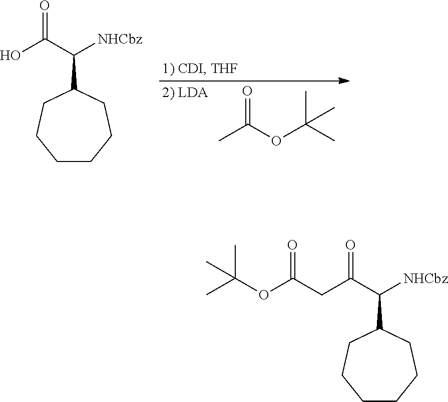

A solution of (S)-2-(((benzyloxy)carbonyl)amino)-2-cycloheptylacetic acid (56.0 g, 183 mmol, 1.00 eq) and 1,1′-carbonyldiimidazole (32.7 g, 201 mmol, 1.10 eq) in tetrahydrofuran (600 mL) was stirred at 0° C. for 2 h. Meanwhile, to a solution of tert-butyl acetate (63.9 g, 550 mmol, 73.7 mL, 3.00 eq) in tetrahydrofuran (500 mL) was added a solution of lithium diisopropylamide (2 M, 275 mL, 3.00 eq) and the reaction mixture was stirred at −78° C. for 2 h. The two reaction mixtures were mixed and stirred at −78° C. for 2 h. The reaction mixture was quenched by the addition of a saturated aqueous solution of ammonium chloride and extracted with ethyl acetate, dried over anhydrous sodium sulfate, filtered, and concentrated under reduced pressure to give a residue which was purified by column chromatography (SiO₂, petroleum ether/ethyl acetate) to afford tert-butyl (S)-4-(((benzyloxy)carbonyl)amino)-4-cycloheptyl-3-oxobutanoate (36.0 g, 89.2 mmol, 48.6% yield) as a white solid. ¹H NMR (400 MHz, CDCl₃) δ 7.39-7.31 (m, 5H), 5.37 (d, J=8.8 Hz, 1H), 5.12 (s, 2H), 4.50-4.47 (dd, J1=3.6, J2=8.8 Hz, 1H), 3.48-3.42 (m, 2H), 2.14-2.06 (m, 1H), 1.85-1.55 (m, 7H), 1.49-1.44 (m, 11H), 1.39-1.13 (m, 3H). LCMS [M+Na]⁺=426.2 m/z.

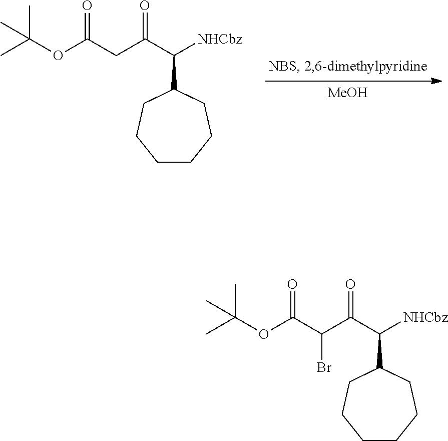 [SMILES: "MeOH" ; CC(C)(C)OC(=O)CC(=O)[C@@H](NC(=O)OCc1ccccc1)C1CCCCCC1 ; CC(C)(C)OC(=O)C(Br)C(=O)[C@@H](NC(=O)OCc1ccccc1)C1CCCCCC1]

To a solution of tert-butyl (S)-4-(((benzyloxy)carbonyl)amino)-4-cycloheptyl-3-oxobutanoate (36.0 g, 89.2 mmol, 1.00 eq) in methanol (400 mL) was added N-bromosuccinimide (13.5 g, 75.8 mmol, 0.85 eq) and 2,6-dimethylpyridine (771 mg, 7.14 mmol, 0.08 eq) at 0° C. The reaction mixture was stirred at 15° C. for 2 h. The reaction mixture was diluted with water and extracted with ethyl acetate. The combined organic layers were washed with a saturated aqueous solution of sodium hydrogen carbonate, dried over anhydrous sodium sulfate, filtered, and concentrated under reduced pressure to afford tert-butyl (4S)-4-(((benzyloxy)carbonyl)amino)-2-bromo-4-cycloheptyl-3-oxobutanoate (40.0 g, 82.9 mmol, 92.9% yield) as a yellow oil.

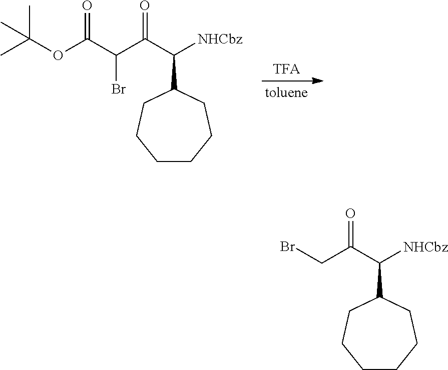 [SMILES: CC(C)(C)OC(=O)C(Br)C(=O)[C@@H](NC(=O)OCc1ccccc1)C1CCCCCC1 ; O=C(N[C@H](C(=O)CBr)C1CCCCCC1)OCc1ccccc1]

To a solution of tert-butyl (4S)-4-(((benzyloxy)carbonyl)amino)-2-bromo-4-cycloheptyl-3-oxobutanoate (40.0 g, 82.9 mmol, 1.00 eq) in toluene (400 mL) was added trifluoroacetic acid (56.7 g, 497 mmol, 36.8 mL, 6.00 eq) at 0° C. The reaction mixture was stirred at 75° C. for 2 h. The reaction mixture was diluted with water and a saturated aqueous solution of sodium hydrogen carbonate was added to adjust the pH=9. The reaction mixture was extracted with dichloromethane and the combined organic layers were dried over anhydrous sodium sulfate, filtered, and concentrated under reduced pressure to give a residue which was purified by prep-HPLC (basic condition; column: Kromasil Eternity XT (250 mm×80 mm, 10 μm); mobile phase: [water (NH₄OH)—acetonitrile]; B %: 55%-85%, 20 min) to afford benzyl (S)-(3-bromo-1-cycloheptyl-2-oxopropyl)carbamate. The title compound was isolated as the first eluting, single stereoisomer by chiral SFC purification (column: Daicel Chiralcel OJ (250 mm×30 mm, 10 μm); mobile phase: [0.1% NH₃H₂O in IPA]; B %: 20%-20%, 3 min); (11.3 g, 29.5 mmol, 35.6% yield) and was obtained as a white solid. ¹H NMR (400 MHz, MeOD) δ 7.39-7.27 (m, 5H), 5.10 (s, 2H), 4.45-4.37 (m, 1H), 4.22 (s, 2H), 2.21-2.04 (m, 1H), 1.70-1.28 (m, 12H). LCMS [M+H]⁺=384.2 m/z.

### Example 5: Preparation of Benzyl ((1S)-3-bromo-1-(3,3-difluorocyclohexyl)-2-oxopropyl)carbamate

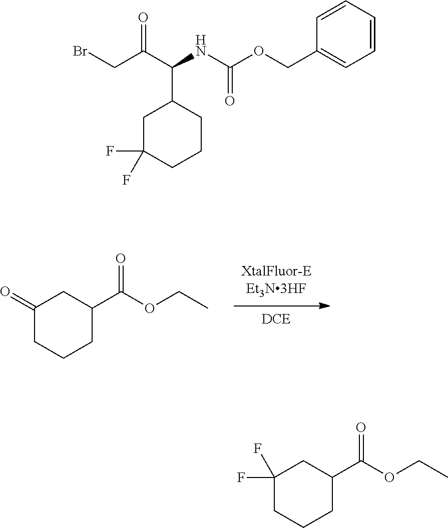 [SMILES: O=C(N[C@H](C(=O)CBr)C1CCCC(F)(F)C1)OCc1ccccc1 ; CCOC(=O)C1CCCC(=O)C1 ; "DCE" ; CCOC(=O)C1CCCC(F)(F)C1]

To a solution of N,N-diethylamino-S,S-difluorosulfinium tetrafluoroborate (5.05 kg, 22.0 mol, 2.50 eq) and triethylamine trihydrofluoride (2.13 kg, 13.2 mol, 2.15 L, 1.50 eq) in 1,2-dichloroethane (25.0 L) was added dropwise ethyl 3-oxocyclohexane-1-carboxylate (1.50 kg, 8.81 mol, 1.00 eq) over a period of 30 min. The reaction mixture was stirred at 80° C. for 3 h. The reaction mixture was poured into a saturated aqueous solution of sodium hydrogen carbonate (15.0 L) at 20° C. and the organic phase was separated. The aqueous phase was extracted with dichloromethane, the combined organic phases were washed with a 10% aqueous solution of citric acid and brine. Upon concentration under reduced pressure to give a residue, a distillation under vacuum (80° C., −0.095 Mpa) was performed to afford ethyl 3,3-difluorocyclohexane-1-carboxylate (1.26 kg, 6.56 mol, 74.38% yield) as a yellow oil. ¹H NMR (400 MHz, CDCl₃) δ 4.15 (q, J=7.2 Hz, 2H), 2.57-2.64 (m, 1H), 2.30-2.37 (m, 1H), 1.99-2.12 (m, 2H), 1.78-1.94 (m, 2H), 1.53-1.73 (m, 2H), 1.35-1.45 (m, 1H), 1.26 (t, J=7.2 Hz, 3H).

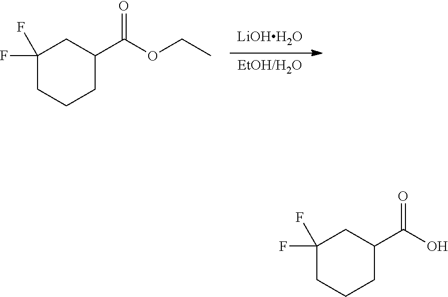

To a solution of ethyl 3,3-difluorocyclohexane-1-carboxylate (1.26 kg, 6.56 mol, 1.00 eq) in ethanol (7.56 L) and water (5.04 L) was added portionwise lithium hydroxide monohydrate (330 g, 7.88 mol, 1.20 eq) at 0° C. The reaction mixture was stirred at 20° C. for 2 h. The reaction mixture was concentrated under reduced pressure. The resulting residue was diluted with a 3M aqueous solution of hydrochloric acid to adjust pH=3 and extracted with dichloromethane. The combined organic phases were dried over anhydrous sodium sulfate, filtered, and concentrated under reduced pressure to give a residue, which was stirred in petroleum ether at room temperature for 5 h to afford 3,3-difluorocyclohexane-1-carboxylic acid (655 g, 3.99 mol) as a white solid. ¹H NMR (400 MHz, CDCl₃) δ 2.66-2.72 (m, 1H), 2.33-2.41 (m, 1H), 2.06-2.14 (m, 2H), 1.81-1.97 (m, 2H), 1.57-1.76 (s, 2H), 1.40-1.49 (m, 1H).

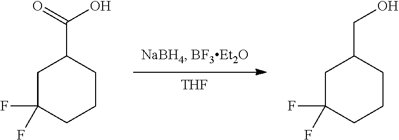 [SMILES: O=C(O)C1CCCC(F)(F)C1 ; OCC1CCCC(F)(F)C1 ; "THF"]

To a solution of 3,3-difluorocyclohexane-1-carboxylic acid (50.0 g, 305 mmol, 1.00 eq) in tetrahydrofuran (600 mL) was added portionwise sodium borohydride (12.7 g, 335 mmol, 1.10 eq) at 0° C. followed by a dropwise addition of boron trifluoride diethyl etherate (43.2 g, 305 mmol, 37.5 mL, 1.00 eq). The reaction mixture was stirred at room temperature for 3 h. The reaction mixture was quenched by the addition of a saturated aqueous solution of ammonium chloride and extracted with ethyl acetate, dried over anhydrous sodium sulfate, filtered, and concentrated under reduced pressure to afford (3,3-difluorocyclohexyl)methanol (45.0 g, 300 mmol, 98.3% yield) as a yellow oil. ¹H NMR (400 MHz, DMSO-d₆) δ 4.56 (t, J=6.4 Hz, 1H), 3.22-3.32 (m, 2H), 2.00-2.07 (m, 1H), 1.93-1.98 (m, 1H), 1.58-1.79 (m, 4H), 1.35-1.51 (m, 2H), 0.95-1.02 (m, 1H).

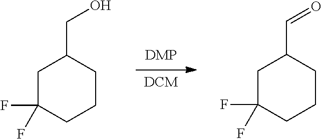

To a solution of (3,3-difluorocyclohexyl)methanol (40.0 g, 266 mmol, 1.00 eq) in dichloromethane (1.00 L) was added Dess-Martin periodinane (124 g, 293 mmol, 90.8 mL, 1.10 eq) at 0° C. The reaction mixture was stirred at 20° C. for 1 h. The reaction mixture was concentrated under reduced pressure and the resulting residue was purified by re-crystallization from petroleum ether at 20° C. to afford 3,3-difluorocyclohexane-1-carbaldehyde (39.0 g, 263 mmol, 98.8% yield) as a yellow oil. ¹H NMR (400 MHz, CDCl₃) δ 9.67 (d, J=1.6 Hz, 1H), 2.62-2.71 (m, 1H), 2.31-2.40 (m, 1H), 1.97-2.06 (m, 2H), 1.75-1.92 (m, 4H), 1.51-1.57 (m, 1H).

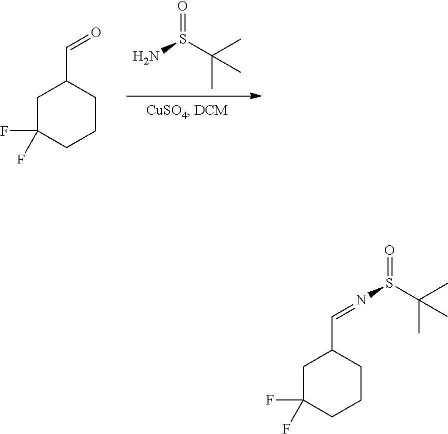

To a solution of 3,3-difluorocyclohexane-1-carbaldehyde (39.0 g, 263 mmol, 1.00 eq) and (S)-2-methylpropane-2-sulfinamide (38.2 g, 316 mmol, 1.20 eq) in dichloromethane (600 mL) was added copper sulfate (42.0 g, 263 mmol, 40.4 mL, 1.00 eq). The reaction mixture was stirred at room temperature for 10 h. The reaction mixture was diluted with water and extracted with dichloromethane. The combined organic layers were washed with brine, dried over anhydrous sodium sulfate, filtered, and concentrated under reduced pressure to afford (S)—N-((3,3-difluorocyclohexyl)methylene)-2-methylpropane-2-sulfinamide (49.0 g, 195 mmol, 74.0% yield) as a yellow oil. ¹H NMR (400 MHz, CDCl₃) δ 8.00-8.02 (m, 1H), 2.75-2.82 (m, 1H), 2.30-2.36 (m, 1H), 2.11-2.13 (m, 1H), 1.87-1.93 (m, 1H), 1.53-1.84 (m, 5H), 1.18 (s, 9H).

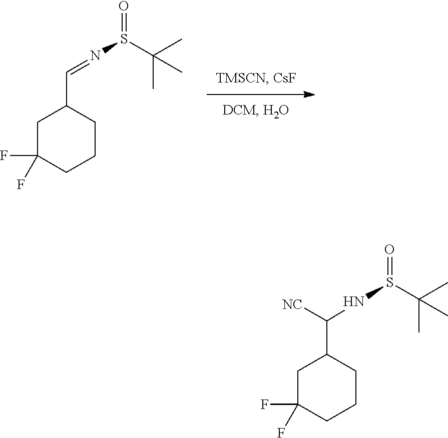 [SMILES: CC(C)(C)[S@](=O)N=CC1CCCC(F)(F)C1 ; CC(C)(C)[S@](=O)NC(C#N)C1CCCC(F)(F)C1]

To a solution of (S)—N-((3,3-difluorocyclohexyl)methylene)-2-methylpropane-2-sulfinamide (49.0 g, 195 mmol, 1.00 eq) in dichloromethane (600 mL) was successively added water (7.00 g, 389 mmol, 7.00 mL, 1.99 eq), cesium fluoride (5.92 g, 39.0 mmol, 1.44 mL, 0.200 eq) and trimethylsilyl cyanide (39.0 g, 390 mmol, 49.0 mL, 2.00 eq). The reaction mixture was stirred at 20° C. for 5 h. The reaction mixture was quenched by the addition of water at 0° C., and an extraction with dichloromethane was performed. The combined organic layers were washed with brine, dried over anhydrous sodium sulfate, filtered, and concentrated under reduced pressure to give a residue which was purified by column chromatography (SiO₂, petroleum ether/ethyl acetate) to afford (S)—N-(cyano(3,3-difluorocyclohexyl)methyl)-2-methylpropane-2-sulfinamide (49.0 g, 176 mmol, 90.2% yield) as a yellow oil. ¹H NMR (400 MHz, CDCl₃) δ 4.11-4.16 (m, 1H), 2.27-2.30 (m, 1H), 2.11-2.20 (m, 2H), 1.89-2.02 (m, 2H), 1.38-1.75 (m, 4H), 1.26 (s, 9H).

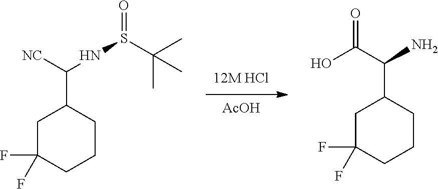 [SMILES: "AcOH" ; CC(C)(C)[S@](=O)NC(C#N)C1CCCC(F)(F)C1 ; N[C@H](C(=O)O)C1CCCC(F)(F)C1]

To a solution of (S)—N-(cyano(3,3-difluorocyclohexyl)methyl)-2-methylpropane-2-sulfinamide (49.0 g, 176 mmol, 1.00 eq) in acetic acid (250 mL) was added hydrochloric acid (12 M, 250 mL, 17.0 eq). The reaction mixture was stirred at 110° C. for 12 h. The reaction mixture was concentrated under reduced pressure to afford (2S)-2-amino-2-(3,3-difluorocyclohexyl)acetic acid (31.0 g, 160 mmol, 91.1% yield) as a brown solid.

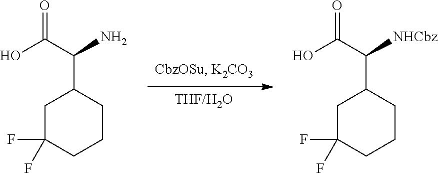 [SMILES: N[C@H](C(=O)O)C1CCCC(F)(F)C1 ; "THF/H2O" ; O=C(N[C@H](C(=O)O)C1CCCC(F)(F)C1)OCc1ccccc1]

To a solution of (2S)-2-amino-2-(3,3-difluorocyclohexyl)acetic acid (31.0 g, 160 mmol, 1.00 eq) in tetrahydrofuran (250 mL) and water (250 mL) was added potassium carbonate (66.5 g, 481 mmol, 3.00 eq) followed by N-(Benzyloxycarbonyloxy)succinimide (44.0 g, 176 mmol, 1.10 eq). The reaction mixture was stirred at 20° C. for 10 h. The reaction mixture was concentrated under reduced pressure and the resulting residue was diluted with a 1M aqueous solution of hydrochloric acid to adjust pH=3, then extracted with dichloromethane. The combined organic layers were washed with brine, dried over anhydrous sodium sulfate, filtered, and concentrated under reduced pressure to give a residue which was purified by prep-HPLC (0.1% FA condition) to afford (2S)-2-(((benzyloxy)carbonyl)amino)-2-(3,3-difluorocyclohexyl)acetic acid (40.0 g, 122 mmol, 76.1% yield) as a white solid. LCMS [M+H]⁺=328.2 m/z.

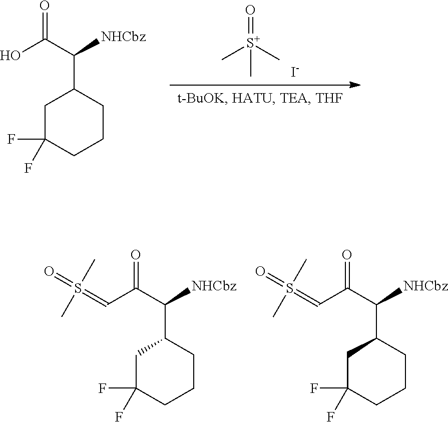

To a solution of trimethylsulfoxonium iodide (66.6 g, 302 mmol, 3.00 eq) in tetrahydrofuran (400 mL) was added a solution of potassium tert-butoxide (33.9 g, 302 mmol, 3.00 eq) in tetrahydrofuran (400 mL). The reaction mixture was stirred at 65° C. for 2 h and then cooled down to 0° C. (Reaction 1).

In parallel, to a solution of (2S)-2-(((benzyloxy)carbonyl)amino)-2-(3,3-difluorocyclohexyl)acetic acid (33.0 g, 101 mmol, 1.00 eq) in tetrahydrofuran (300 mL) was added triethylamine (13.3 g, 131 mmol, 18.2 mL, 1.30 eq) followed by 1-[bis(dimethylamino)methylene]-1H-1,2,3-triazolo[4,5-b]pyridinium 3-oxide hexafluorophosphate (HATU, 49.8 g, 131 mmol, 1.30 eq) at 0° C. The reaction mixture was stirred at room temperature for 2 h. The suspension was then filtered, and the filtrate was cooled down to 0° C. (Reaction 2).

The content of the Reaction 2 was slowly added to the Reaction 1 at 0° C. and the resulting mixture was further stirred at 0° C. for 2 h. The reaction mixture was quenched by the addition of water and then diluted with ethyl acetate. An extraction was performed with ethyl acetate. The combined organic layers were dried over anhydrous sodium sulfate, filtered, and concentrated under reduced pressure to give a residue, which was purified by prep-HPLC (0.1% FA condition) to afford benzyl ((1S)-1-(3,3-difluorocyclohexyl)-3-(dimethyl(oxo)-λ⁶-sulfaneylidene)-2-oxopropyl)carbamate (20.0 g, 49.2 mmol, 48.7% yield) as a white solid. LCMS [M+H]⁺=402.3 m/z.

Benzyl ((S)-1-((R)-3,3-difluorocyclohexyl)-3-(dimethyl(oxo)-λ⁶-sulfaneylidene)-2-oxopropyl)carbamate was isolated as the first eluting, single stereoisomer by chiral SFC purification (column: Daicel Chiralpak IF (250 mm×30 mm, 10 μm); mobile phase: [CO₂-0.1% NH₃·H₂O in EtOH]; B %: 35%); (9.00 g, 22.4 mmol, 47.3% yield) and was obtained as a white solid. LCMS [M+H]⁺=402.2 m/z.

Benzyl ((S)-1-((S)-3,3-difluorocyclohexyl)-3-(dimethyl(oxo)-λ⁶-sulfaneylidene)-2-oxopropyl)carbamate was isolated as the second eluting, single stereoisomer by chiral SFC purification (column: Daicel Chiralpak IF (250 mm×30 mm, 10 μm); mobile phase: [CO₂-0.1% NH₃·H₂O in EtOH]; B %: 35%); (9.00 g, 22.4 mmol, 47.3% yield) and was obtained as a white solid. LCMS [M+H]⁺=402.2 m/z.

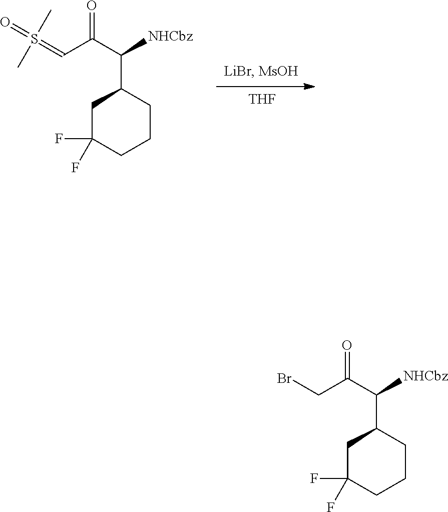 [SMILES: CS(C)(=O)=CC(=O)[C@@H](NC(=O)OCc1ccccc1)[C@H]1CCCC(F)(F)C1 ; O=C(N[C@H](C(=O)CBr)[C@H]1CCCC(F)(F)C1)OCc1ccccc1 ; "THF"]

To a solution of benzyl ((S)-1-((S)-3,3-difluorocyclohexyl)-3-(dimethyl(oxo)-λ⁶-sulfaneylidene)-2-oxopropyl)carbamate (4.00 g, 9.96 mmol, 1.00 eq) in tetrahydrofuran (15.0 mL) was added lithium bromide (1.30 g, 14.9 mmol, 375 μL, 1.50 eq) followed by methanesulfonic acid (1.44 g, 14.9 mmol, 1.07 mL, 1.50 eq). The reaction mixture was stirred at 45° C. for 1 h. The reaction mixture was filtered and concentrated under reduced pressure to give a residue, which was purified by column chromatography (SiO₂, petroleum ether/ethyl acetate) to afford benzyl ((S)-3-bromo-1-((S)-3,3-difluorocyclohexyl)-2-oxopropyl)carbamate (3.30 g, 8.16 mmol, 81.9% yield) as a white solid. ¹H NMR (400 MHz, CDCl₃) δ 7.27-7.41 (m, 5H), 5.34-5.42 (m, 1H), 5.12 (s, 2H), 4.71-4.79 (m, 1H), 3.99-4.08 (m, 1H), 3.77 (s, 1H), 2.12-2.15 (m, 2H), 1.75-1.84 (m, 2H), 1.59-1.67 (m, 1H), 1.38-1.55 (m, 2H), 0.82-1.29 (m, 2H).

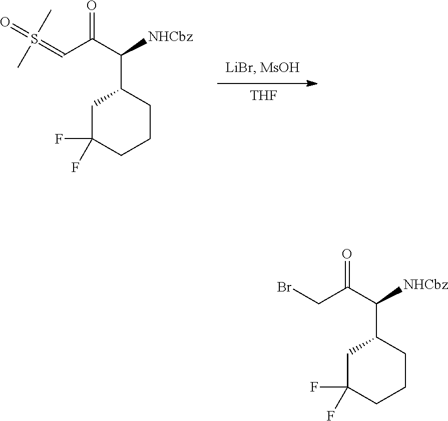 [SMILES: CS(C)(=O)=CC(=O)[C@@H](NC(=O)OCc1ccccc1)[C@@H]1CCCC(F)(F)C1 ; O=C(N[C@H](C(=O)CBr)[C@@H]1CCCC(F)(F)C1)OCc1ccccc1 ; "THF"]

To a solution of benzyl ((S)-1-((R)-3,3-difluorocyclohexyl)-3-(dimethyl(oxo)-λ⁶-sulfaneylidene)-2-oxopropyl)carbamate (5.00 g, 12.45 mmol, 1.00 eq) in tetrahydrofuran (30.0 mL) was added lithium bromide (1.60 g, 18.7 mmol, 471 μL, 1.50 eq) followed by methanesulfonic acid (1.81 g, 18.7 mmol, 1.34 mL, 1.50 eq). The reaction mixture was stirred at 45° C. for 1 h. The reaction mixture was filtered and concentrated under reduced pressure to give a residue, which was purified by column chromatography (SiO₂, petroleum ether/ethyl acetate) to afford benzyl ((S)-3-bromo-1-((R)-3,3-difluorocyclohexyl)-2-oxopropyl)carbamate (3.40 g, 8.41 mmol, 67.0% yield) as a white solid. ¹H NMR (400 MHz, CDCl₃) δ 7.33-7.41 (m, 5H), 5.35-5.39 (m, 1H), 5.13 (s, 2H), 4.71-4.79 (m, 1H), 3.99-4.07 (m, 2H), 2.21-2.26 (m, 2H), 1.81-1.86 (m, 1H), 1.45-1.67 (m, 5H), 0.98-1.08 (m, 1H).

### Example 6: Preparation of 1-(tert-butyl) 3-methyl (5R)-2-oxo-5-(trifluoromethyl)piperidine 1,3-dicarboxylate

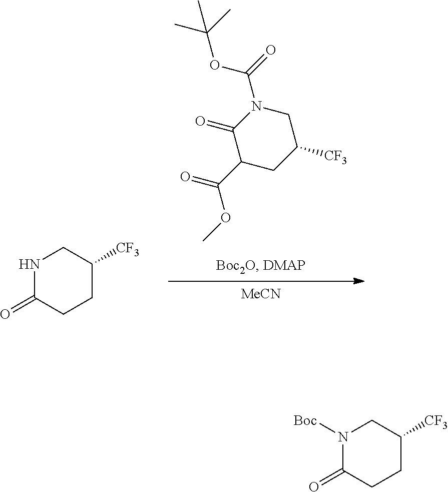 [SMILES: "MeCN" ; COC(=O)C1C[C@@H](C(F)(F)F)CN(C(=O)OC(C)(C)C)C1=O ; O=C1CC[C@@H](C(F)(F)F)CN1 ; CC(C)(C)OC(=O)N1C[C@H](C(F)(F)F)CCC1=O]

To a solution of (R)-5-(trifluoromethyl)piperidin-2-one (6.60 g, 39.5 mmol, 1.00 eq) in acetonitrile (50.0 mL) was added successively di-tert-butyl dicarbonate (10.3 g, 47.4 mmol, 10.9 mL, 1.20 eq) and 4-dimethylaminopyridine (965 mg, 7.90 mmol, 0.20 eq). The reaction mixture was stirred at room temperature for 3 h. The reaction mixture was concentrated under reduced pressure to give a residue, which was purified by column chromatography (SiO₂, petroleum ether:ethyl acetate) to afford tert-butyl (R)-2-oxo-5-(trifluoromethyl)piperidine-1-carboxylate (8.00 g, 29.9 mmol, 75.8% yield) as a white solid. LCMS [M+Na]⁺=290.1 m/z.

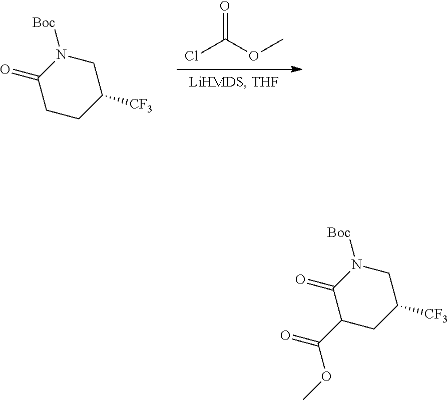

To a solution of tert-butyl (R)-2-oxo-5-(trifluoromethyl)piperidine-1-carboxylate (8.00 g, 29.9 mmol, 1.00 eq) in tetrahydrofuran (100 mL) was added a solution of lithium bis(trimethylsilyl)amide (1 M, 59.9 mL, 2.00 eq) at −78° C. The reaction mixture was stirred at −78° C. for 1 h and methyl chloroformate (5.45 g, 57.7 mmol, 4.47 mL, 1.93 eq) was then added. The reaction mixture was stirred at −78° C. for 2 h. The reaction mixture was quenched by the addition of a saturated aqueous solution of ammonium chloride at 0° C. and then extracted with ethyl acetate. The combined organic layers were dried over anhydrous sodium sulfate, filtered, and concentrated under reduced pressure to give a residue, which was purified by column chromatography (SiO₂, petroleum ether:ethyl acetate) to afford 1-(tert-butyl) 3-methyl (5R)-2-oxo-5-(trifluoromethyl)piperidine-1,3-dicarboxylate (7.00 g, 21.5 mmol, 71.9% yield) as a white solid. LCMS [2M+Na]⁺=673.2 m/z.

### Example 7: Preparation of 1-(tert-butyl) 3-methyl (5S)-2-oxo-5-(trifluoromethyl)piperidine-1,3-dicarboxylate

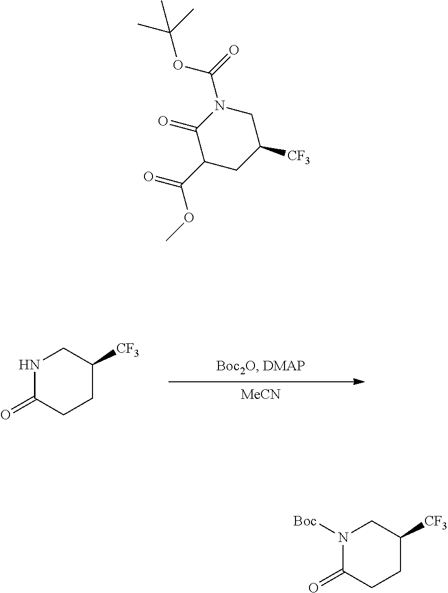 [SMILES: "MeCN" ; COC(=O)C1C[C@H](C(F)(F)F)CN(C(=O)OC(C)(C)C)C1=O ; O=C1CC[C@H](C(F)(F)F)CN1 ; CC(C)(C)OC(=O)N1C[C@@H](C(F)(F)F)CCC1=O]

To a solution of (S)-5-(trifluoromethyl)piperidin-2-one (1.50 g, 8.98 mmol, 1.00 eq) in acetonitrile (10.0 mL) was added successively di-tert-butyl dicarbonate (2.94 g, 13.4 mmol, 3.09 mL, 1.50 eq) and 4-dimethylaminopyridine (219 mg, 1.80 mmol, 0.20 eq). The reaction mixture was stirred at room temperature for 12 h. The reaction mixture was concentrated under reduced pressure to give a residue, which was purified by column chromatography (SiO₂, petroleum ether:ethyl acetate) to afford tert-butyl (S)-2-oxo-5-(trifluoromethyl)piperidine-1-carboxylate (1.30 g, 4.86 mmol, 54.2% yield) as a white solid. ¹H NMR (400 MHz, CDCl₃) δ 3.99 (dd, J1=5.8, J2=13.6 Hz, 1H), 3.75 (dd, J=8.4, 13.6 Hz, 1H), 2.77-2.42 (m, 3H), 2.18-2.11 (m, 1H), 2.00-1.86 (m, 1H), 1.53 (s, 9H).

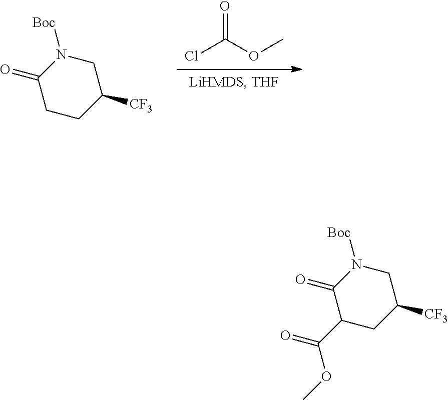

To a solution of tert-butyl (S)-2-oxo-5-(trifluoromethyl)piperidine-1-carboxylate (1.30 g, 4.86 mmol, 1.00 eq) in tetrahydrofuran (10 mL) was added a solution of lithium bis(trimethylsilyl)amide (1 M, 7.30 mL, 1.50 eq) at −70° C. The reaction mixture was stirred at −70° C. for 30 min and methyl chloroformate (689 mg, 7.30 mmol, 565 μL, 1.50 eq) was then added. The reaction mixture was stirred at −70° C. for 1 h. The reaction mixture was quenched by the addition of a saturated aqueous solution of ammonium chloride at 0° C. and then extracted with ethyl acetate. The combined organic layers were dried over anhydrous sodium sulfate, filtered, and concentrated under reduced pressure to give a residue, which was purified by column chromatography (SiO₂, petroleum ether:ethyl acetate) to afford 1-(tert-butyl) 3-methyl (5S)-2-oxo-5-(trifluoromethyl)piperidine-1,3-dicarboxylate (1.30 g, 4.00 mmol, 82.1% yield) as a white solid. ¹H NMR (400 MHz, CDCl₃) δ 4.03-3.93 (m, 1H), 3.88-3.97 (m, 3H), 3.77-3.49 (m, 1H), 2.95-2.58 (m, 1H), 2.57-2.08 (m, 2H), 1.53 (d, J=2.4 Hz, 9H). LCMS [2M+Na]⁺=673.2 m/z.

### Example 8: Preparation of (3R,5R)-3-((3-amino-1,2,4-triazin-6-yl)methyl)-5-(trifluoromethyl)piperidin-2-one

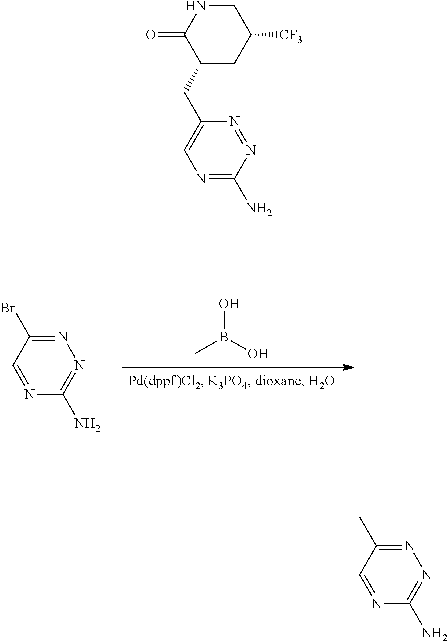

To a solution of 6-bromo-1,2,4-triazin-3-amine (20.0 g, 114 mmol, 1.00 eq) and methylboronic acid (34.2 g, 571 mmol, 5.00 eq) in dioxane (200 mL) and water (40.0 mL) was added successively [1,1′-Bis(diphenylphosphino)ferrocene]dichloropalladium(II) (8.36 g, 11.4 mmol, 0.100 eq) and potassium phosphate tribasic (72.8 g, 343 mmol, 3.00 eq). The reaction mixture was degassed and purged with nitrogen (3 cycles), and then stirred at 100° C. for 12 h under nitrogen atmosphere. The reaction mixture was cooled down to room temperature, filtered, and concentrated under reduced pressure to afford 6-methyl-1,2,4-triazin-3-amine (35.0 g, crude) as a brown oil. LCMS [M+H]⁺=111.4 m/z.

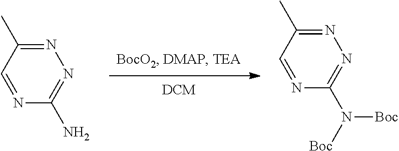

To a solution of 6-methyl-1,2,4-triazin-3-amine (35.0 g, 318 mmol, 1.00 eq) in dichloromethane (300 mL) was successively added 4-dimethylaminopyridine (7.77 g, 63.6 mmol, 0.20 eq), di-tert-butyl dicarbonate (173 g, 795 mmol, 182 mL, 2.50 eq) and triethylamine (48.2 g, 477 mmol, 66.4 mL, 1.50 eq). The reaction mixture was stirred at room temperature for 4 h. The reaction mixture was diluted with water and extracted with dichloromethane/methanol (10/1). The combined organic layers were washed with brine, dried over anhydrous sodium sulfate, filtered, and concentrated under reduced pressure to give a residue, which was purified by column chromatography (SiO₂, petroleum ether/ethyl acetate) to afford tert-butyl (tert-butoxycarbonyl)(6-methyl-1,2,4-triazin-3-yl)carbamate (14.2 g, 45.8 mmol, 14.4% yield) as a yellow solid. ¹H NMR (400 MHz, CDCl₃) δ 8.52 (s, 1H), 2.78 (s, 3H), 1.44-1.46 (m, 18H). LCMS [2M+Na]⁺=643.3 m/z.

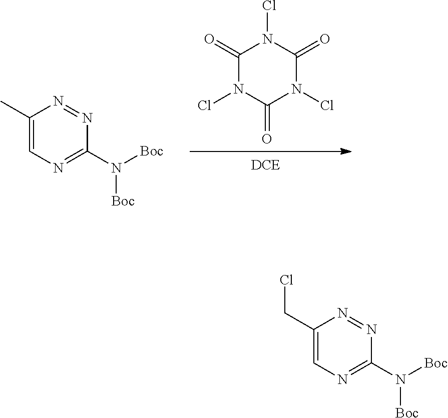

To a solution of tert-butyl (tert-butoxycarbonyl)(6-methyl-1,2,4-triazin-3-yl)carbamate (15.0 g, 48.3 mmol, 1.00 eq) in dichloroethane (50.0 mL) was added trichloroisocyanuric acid (4.49 g, 19.3 mmol, 0.40 eq). The reaction mixture was stirred at 90° C. for 4 h. The reaction mixture was diluted with water and extracted with dichloromethane. The combined organic layers were washed with brine, dried over anhydrous sodium sulfate, filtered, and concentrated under reduced pressure to give a residue, which was purified by column chromatography (SiO₂, petroleum ether/ethyl acetate) to afford tert-butyl (tert-butoxycarbonyl)(6-(chloromethyl)-1,2,4-triazin-3-yl)carbamate (2.70 g, 7.83 mmol, 16.2% yield) as a yellow solid. ¹H NMR (400 MHz, CDCl₃) δ 8.85 (s, 1H), 4.94 (s, 2H), 1.47 (s, 18H). LCMS [2M+Na]⁺=711.1 m/z.

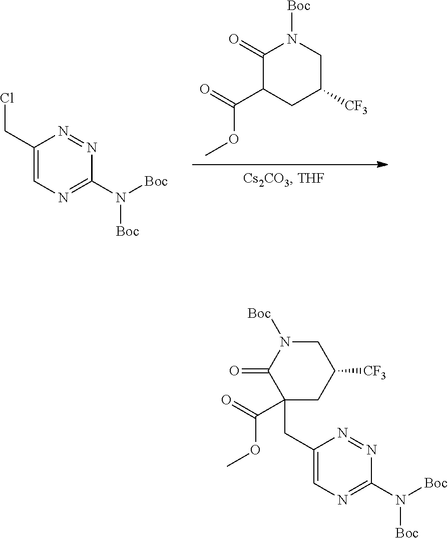

To a solution of 1-(tert-butyl) 3-methyl (3R,5R)-2-oxo-5-(trifluoromethyl)piperidine-1,3-dicarboxylate (2.83 g, 8.70 mmol, 1.20 eq) in tetrahydrofuran (20.00 mL) was added cesium carbonate (4.72 g, 14.5 mmol, 2.00 eq). The reaction mixture was stirred at room temperature for 30 minutes prior to the addition of tert-butyl (tert-butoxycarbonyl)(6-(chloromethyl)-1,2,4-triazin-3-yl)carbamate (2.50 g, 7.25 mmol, 1.00 eq). The reaction mixture was further stirred at 50° C. for 1 h. The reaction mixture was cooled down to room temperature, diluted with water and extracted with ethyl acetate. The combined organic layers were washed with brine, dried over anhydrous sodium sulfate, filtered, and concentrated under reduced pressure to give a residue, which was purified by column chromatography (SiO₂, petroleum ether/ethyl acetate) to afford 1-(tert-butyl) 3-methyl (3R,5R)-3-((3-(bis(tert-butoxycarbonyl)amino)-1,2,4-triazin-6-yl)methyl)-2-oxo-5-(trifluoromethyl)piperidine-1,3-dicarboxylate (4.00 g, 6.31 mmol, 87.1% yield) as a yellow oil. ¹H NMR (400 MHz, CDCl₃) δ 8.73-8.78 (m, 1H), 4.24-4.38 (m, 1H), 3.79-3.80 (m, 3H), 3.60-3.66 (m, 2H), 3.36-3.45 (m, 1H), 2.85-2.95 (m, 1H), 2.60-2.68 (m, 1H), 2.28-2.37 (m, 1H), 1.53-1.54 (m, 9H), 1.43-1.45 (m, 18H). LCMS [M+H]⁺=634.3 m/z.

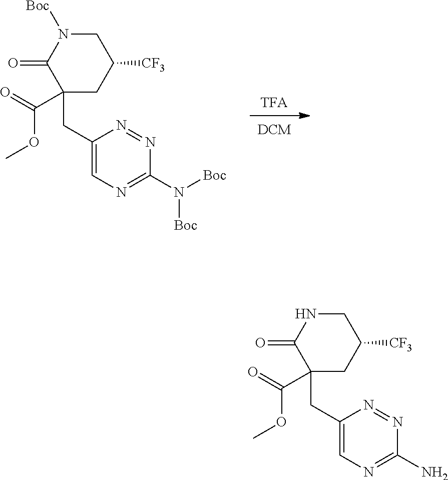

To a solution of 1-(tert-butyl) 3-methyl (3R,5R)-3-((3-(bis(tert-butoxycarbonyl)amino)-1,2,4-triazin-6-yl)methyl)-2-oxo-5-(trifluoromethyl)piperidine-1,3-dicarboxylate (2.00 g, 2.73 mmol, 1.00 eq) in dichloromethane (20.0 mL) was added trifluoroacetic acid (3.11 g, 27.3 mmol, 2.02 mL, 10.0 eq). The reaction mixture was stirred at room temperature for 2 h. The reaction mixture was concentrated under reduced pressure and was quenched with a saturated aqueous solution of sodium hydrogen carbonate. An extraction was performed with dichloromethane/methanol (8/1). The combined organic layers were washed with brine, dried over anhydrous sodium sulfate, filtered, and concentrated under reduced pressure to afford methyl (3R,5R)-3-((3-amino-1,2,4-triazin-6-yl)methyl)-2-oxo-5-(trifluoromethyl)piperidine-3-carboxylate (0.65 g, TFA salt) as a yellow oil. LCMS [M+H]⁺=334.3 m/z.

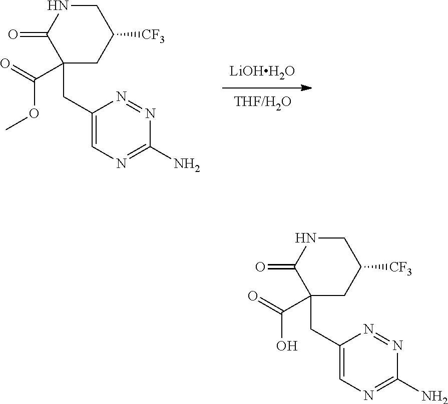 [SMILES: COC(=O)C1(Cc2cnc(N)nn2)C[C@@H](C(F)(F)F)CNC1=O ; "LiOH·H2O" ; "THF/H2O" ; Nc1ncc(CC2(C(=O)O)C[C@@H](C(F)(F)F)CNC2=O)nn1]

To a solution of methyl (3R,5R)-3-((3-amino-1,2,4-triazin-6-yl)methyl)-2-oxo-5-(trifluoromethyl)piperidine-3-carboxylate (200 g, 356 mmol, 1.00 eq, TFA salt) in THF (2.00 L) and water (1.00 L) was added LiOH·H₂O (59.8 g, 1.43 mol, 4.00 eq). The reaction mixture was stirred at 20° C. for 8 h. The reaction mixture was adjusted to pH=3~4 with 1M HCl and extracted with ethyl acetate. The combined organic layers were washed with water and the combined organic layers were concentrated under reduced pressure to give a residue. The aqueous layer was lyophilized to afford (3R,5R)-3-((3-amino-1,2,4-triazin-6-yl)methyl)-2-oxo-5-(trifluoromethyl)piperidine-3-carboxylic acid (140 g, crude) as a yellow solid. LCMS [M+H]⁺=320.1 m/z.

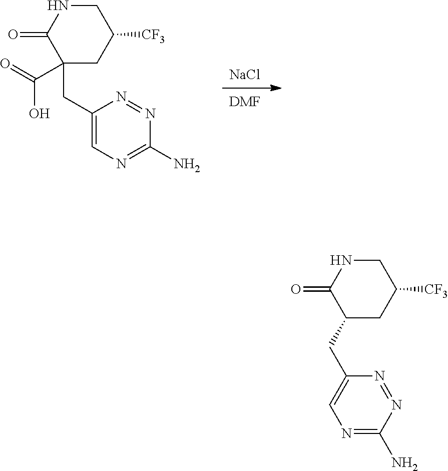

To a solution of (3R,5R)-3-((3-amino-1,2,4-triazin-6-yl)methyl)-2-oxo-5-(trifluoromethyl)piperidine-3-carboxylic acid (140 g, 439 mmol, 1.00 eq) in N,N-dimethylformamide (1.50 L) was added NaCl (76.9 g, 1.32 mol, 3.00 eq). The reaction mixture was stirred at 100° C. for 3 h. The reaction mixture was concentrated under reduced pressure to give a residue, which was purified by reversed phase-HPLC (column: Agela-H1000GC500 YMC-Triart Prep C18 15 μm 120A; Flow rate: 400 ml/min; Mobile phase: MeCN/H₂O; Gradient B %: 0-20% 45 min; Instrument: I.D.100 mm*400 mm) to afford (3R,5R)-3-((3-amino-1,2,4-triazin-6-yl)methyl)-5-(trifluoromethyl)piperidin-2-one (55.0 g, 200 mmol, 27.5% yield) as a yellow solid.

The title compound was isolated as the second eluting, single stereoisomer by chiral SFC purification (column: Daicel Chiralpak AY (250 mm×50 mm, 10 μm); mobile phase: [CO₂-EtOH(0.1% NH₃·H₂O)]; B %: 45%, isocratic elution mode); (38.1 g, 134 mmol, 67.0% yield) and was obtained as a light yellow solid. ¹H NMR (400 MHz, D₂O) δ 8.28 (s, 1H), 3.48-3.47 (m, 1H), 3.35-3.24 (m, 2H), 2.99-2.91 (m, 3H), 2.11-2.08 (m, 1H), 1.73-1.64 (m, 1H). LCMS [M+H]⁺=276.1 m/z.

### Example 9: Preparation of Benzyl (S)-(3-bromo-1-(4,4-difluorocyclohexyl)-2-oxopropyl)carbamate (Compound 1 and Compound 2)

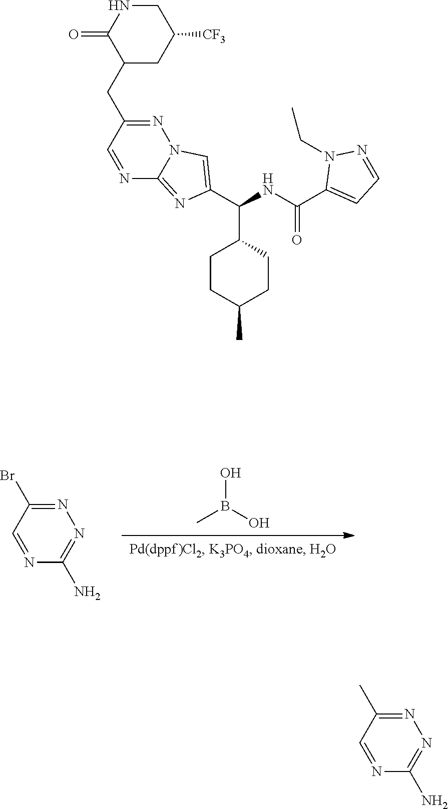

To a solution of 6-bromo-1,2,4-triazin-3-amine (20.0 g, 114 mmol, 1.00 eq) and methylboronic acid (34.2 g, 571 mmol, 5.00 eq) in dioxane (200 mL) and water (40.0 mL) was added successively [1,1′-bis(diphenylphosphino)ferrocene]dichloropalladium(II) (8.36 g, 11.4 mmol, 0.100 eq) and potassium phosphate tribasic (72.8 g, 343 mmol, 3.00 eq). The reaction mixture was degassed and purged with nitrogen (3 cycles), and then stirred at 100° C. for 12 h under nitrogen atmosphere. The reaction mixture was cooled to RT, filtered, and concentrated under reduced pressure to afford 6-methyl-1,2,4-triazin-3-amine (35.0 g, crude) as a brown oil. LCMS [M+H]⁺=111.4 m/z.

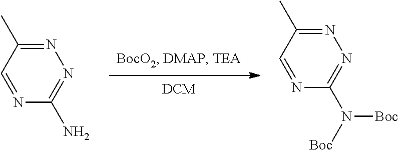

To a solution of 6-methyl-1,2,4-triazin-3-amine (35.0 g, 318 mmol, 1.00 eq) in DCM (300 mL) was successively added 4-dimethylaminopyridine (7.77 g, 63.6 mmol, 0.20 eq), di-tert-butyl dicarbonate (173 g, 795 mmol, 182 mL, 2.50 eq) and triethylamine (48.2 g, 477 mmol, 66.4 mL, 1.50 eq). The reaction mixture was stirred at 25° C. for 4 h. The reaction mixture was diluted with water and extracted with DCM/MeOH (10/1). The combined organic layers were washed with brine, dried over anhydrous sodium sulfate, filtered, and concentrated under reduced pressure to give a residue, which was purified by column chromatography (SiO₂, petroleum ether/EtOAc) to afford tert-butyl (tert-butoxycarbonyl)(6-methyl-1,2,4-triazin-3-yl)carbamate (14.2 g, 45.8 mmol, 14.4% yield) as a yellow solid. ¹H NMR (400 MHz, CDCl₃) d 8.52 (s, 1H), 2.78 (s, 3H), 1.44-1.46 (m, 18H). LCMS [2M+Na]⁺=643.3 m/z.

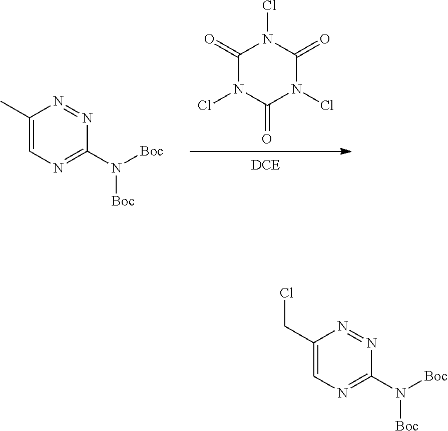

To a solution of tert-butyl (tert-butoxycarbonyl)(6-methyl-1,2,4-triazin-3-yl)carbamate (15.0 g, 48.3 mmol, 1.00 eq) in DCE (50.0 mL) was added trichloroisocyanuric acid (4.49 g, 19.3 mmol, 0.40 eq). The reaction mixture was stirred at 90° C. for 4 h. The reaction mixture was diluted with water and extracted with DCM. The combined organic layers were washed with brine, dried over anhydrous sodium sulfate, filtered, and concentrated under reduced pressure to give a residue, which was purified by column chromatography (SiO₂, petroleum ether/EtOAc) to afford tert-butyl (tert-butoxycarbonyl)(6-(chloromethyl)-1,2,4-triazin-3-yl)carbamate (2.70 g, 7.83 mmol, 16.2% yield) as a yellow solid. ¹H NMR (400 MHz, CDCl₃) d 8.85 (s, 1H), 4.94 (s, 2H), 1.47 (s, 18H). LCMS [2M+Na]⁺=711.1 m/z.

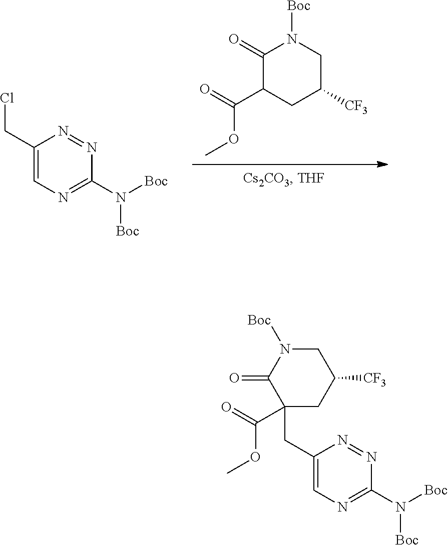

To a solution of 1-(tert-butyl) 3-methyl (5R)-2-oxo-5-(trifluoromethyl)piperidine-1,3-dicarboxylate (2.83 g, 8.70 mmol, 1.20 eq) in THF (20 mL) was added cesium carbonate (4.72 g, 14.5 mmol, 2.00 eq). The reaction mixture was stirred at 25° C. for 30 min prior to the addition of tert-butyl (tert-butoxycarbonyl)(6-(chloromethyl)-1,2,4-triazin-3-yl)carbamate (2.50 g, 7.25 mmol, 1.00 eq). The reaction mixture was further stirred at 50° C. for 1 h. The reaction mixture was cooled to RT, diluted with water, and extracted with EtOAc. The combined organic layers were washed with brine, dried over anhydrous sodium sulfate, filtered, and concentrated under reduced pressure to give a residue, which was purified by column chromatography (SiO₂, petroleum ether/EtOAc) to afford 1-(tert-butyl) 3-methyl (5R)-3-((3-(bis(tert-butoxycarbonyl)amino)-1,2,4-triazin-6-yl)methyl)-2-oxo-5-(trifluoromethyl)piperidine-1,3-dicarboxylate (4.00 g, 6.31 mmol, 87.1% yield) as a yellow oil. ¹H NMR (400 MHz, CDCl₃) d 8.73-8.78 (m, 1H), 4.24-4.38 (m, 1H), 3.79-3.80 (m, 3H), 3.60-3.66 (m, 2H), 3.36-3.45 (m, 1H), 2.85-2.95 (m, 1H), 2.60-2.68 (m, 1H), 2.28-2.37 (m, 1H), 1.53-1.54 (m, 9H), 1.43-1.45 (m, 18H). LCMS [M+H]⁺=634.3 m/z.

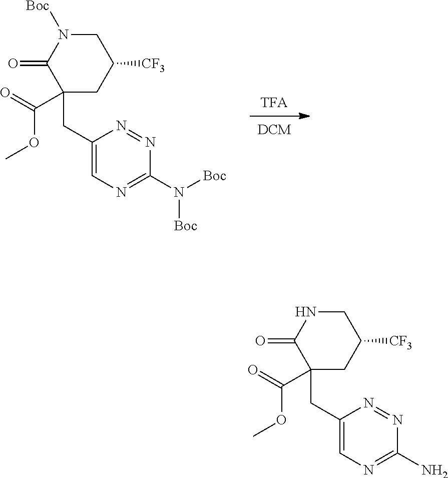

To a solution of 1-(tert-butyl) 3-methyl (5R)-3-((3-(bis(tert-butoxycarbonyl)amino)-1,2,4-triazin-6-yl)methyl)-2-oxo-5-(trifluoromethyl)piperidine-1,3-dicarboxylate (2.00 g, 2.73 mmol, 1.00 eq) in DCM (20.0 mL) was added trifluoroacetic acid (3.11 g, 27.3 mmol, 2.02 mL, 10.0 eq). The reaction mixture was stirred at 25° C. for 2 h. The reaction mixture was concentrated under reduced pressure and diluted with a saturated aqueous solution of sodium hydrogen carbonate. An extraction was performed with DCM/MeOH (8/1). The combined organic layers were washed with brine, dried over anhydrous sodium sulfate, filtered, and concentrated under reduced pressure to afford methyl (5R)-3-((3-amino-1,2,4-triazin-6-yl)methyl)-2-oxo-5-(trifluoromethyl)piperidine-3-carboxylate (0.65 g, crude) as a yellow oil. LCMS [M+H]⁺=334.3 m/z.

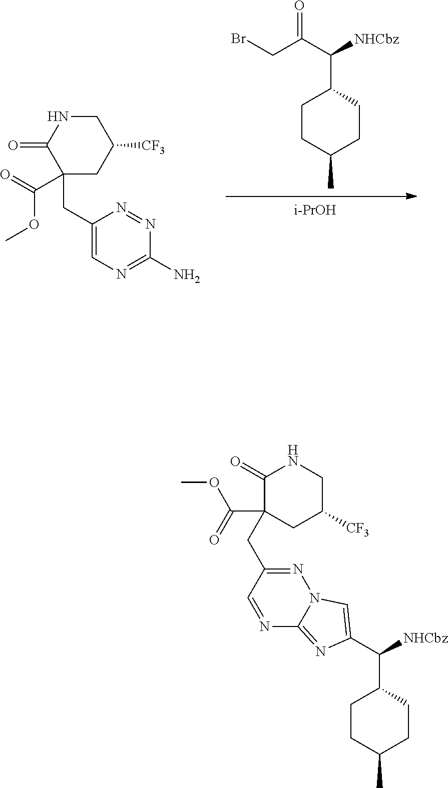 [SMILES: "i-PrOH" ; COC(=O)C1(Cc2cnc(N)nn2)C[C@@H](C(F)(F)F)CNC1=O ; C[C@H]1CC[C@H]([C@H](NC(=O)OCc2ccccc2)C(=O)CBr)CC1 ; COC(=O)C1(Cc2cnc3nc([C@@H](NC(=O)OCc4ccccc4)[C@H]4CC[C@H](C)CC4)cn3n2)C[C@@H](C(F)(F)F)CNC1=O]

To a solution of methyl (5R)-3-((3-amino-1,2,4-triazin-6-yl)methyl)-2-oxo-5-(trifluoromethyl)piperidine-3-carboxylate (250 mg, 750 μmol, 1.00 eq) in isopropanol (5.00 mL) was added benzyl ((S)-3-bromo-1-((1R,4S)-4-methylcyclohexyl)-2-oxopropyl)carbamate (574 mg, 1.50 mmol, 2.00 eq). The reaction mixture was stirred at 70° C. for 5 h. The reaction mixture was cooled to RT and diluted with a saturated aqueous solution of sodium hydrogen carbonate. An extraction was performed with DCM/MeOH (10/1). The combined organic layers were washed with brine, dried over anhydrous sodium sulfate, filtered, and concentrated under reduced pressure to give a residue, which was purified by prep-TLC (SiO₂, petroleum ether/EtOAc) to afford methyl (5R)-3-((6-((S)-(((benzyloxy)carbonyl)amino)((1R,4S)-4-methylcyclohexyl)methyl)imidazo[1,2-b][1,2,4]triazin-2-yl)methyl)-2-oxo-5-(trifluoromethyl)piperidine-3-carboxylate (120 mg, 195 μmol, 25.9% yield) as a yellow oil. LCMS [M+H]⁺=617.3 m/z.

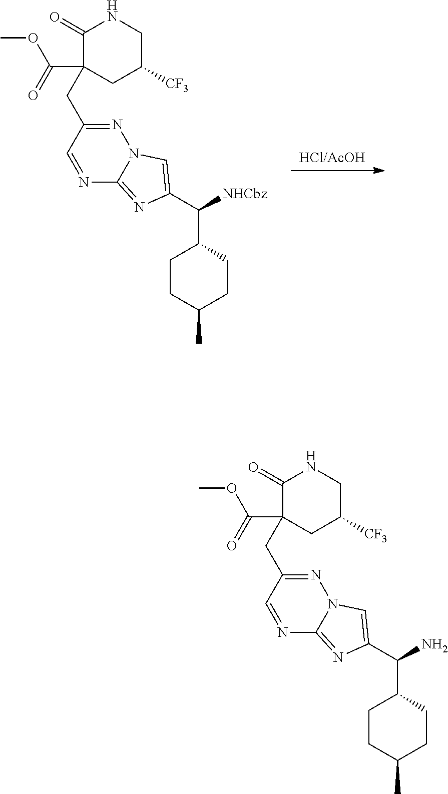 [SMILES: COC(=O)C1(Cc2cnc3nc([C@@H](NC(=O)OCc4ccccc4)[C@H]4CC[C@H](C)CC4)cn3n2)C[C@@H](C(F)(F)F)CNC1=O ; "HCl/AcOH" ; COC(=O)C1(Cc2cnc3nc([C@@H](N)[C@H]4CC[C@H](C)CC4)cn3n2)C[C@@H](C(F)(F)F)CNC1=O]

To a solution of methyl (5R)-3-((6-((S)-(((benzyloxy)carbonyl)amino)((1R,4S)-4-methylcyclohexyl)methyl)imidazo[1,2-b][1,2,4]triazin-2-yl)methyl)-2-oxo-5-(trifluoromethyl)piperidine-3-carboxylate (120 mg, 195 μmol, 1.00 eq) in HCl (12.0 M, 1.00 mL, 61.7 eq) was added acetic acid (0.10 mL). The reaction mixture was stirred at 60° C. for 30 min. The reaction mixture was cooled to RT and the pH was adjusted to pH=9 with a saturated aqueous solution of sodium hydrogen carbonate and was extracted with EtOAc. The combined organic layers were dried over anhydrous sodium sulfate, filtered and concentrated under reduced pressure to afford methyl (5R)-3-((6-((S)-amino((1R,4S)-4-methylcyclohexyl)methyl)imidazo[1,2-b][1,2,4]triazin-2-yl)methyl)-2-oxo-5-(trifluoromethyl)piperidine-3-carboxylate (60.0 mg, 124 μmol, 63.9% yield) as a yellow oil. LCMS [M+H]⁺=483.2 m/z.

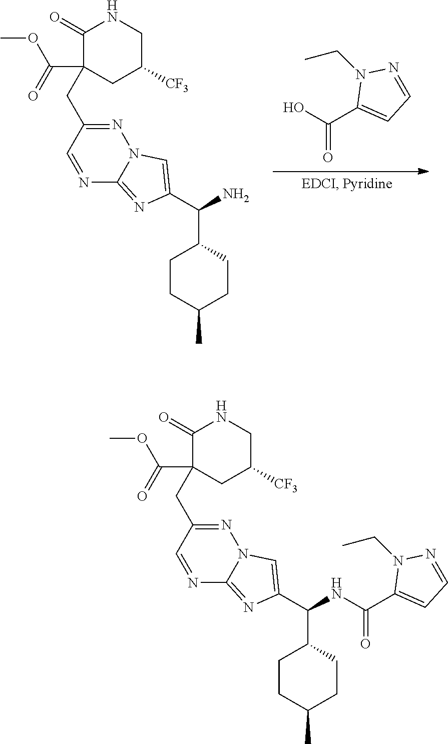

To a solution of methyl (5R)-3-((6-((S)-amino((1R,4S)-4-methylcyclohexyl)methyl)imidazo[1,2-b][1,2,4]triazin-2-yl)methyl)-2-oxo-5-(trifluoromethyl)piperidine-3-carboxylate (65.0 mg, 135 μmol, 1.00 eq), 1-ethyl-1H-pyrazole-5-carboxylic acid (22.6 mg, 162 μmol, 1.20 eq) in pyridine (2.00 mL) was added EDCI (103 mg, 539 mol, 4.00 eq). The reaction mixture was stirred at 25° C. for 2 h. The reaction mixture was diluted with water and extracted with DCM/MeOH (10/1). The combined organic layers were washed with brine, dried over anhydrous sodium sulfate, filtered, and concentrated under reduced pressure to afford methyl (5R)-3-((6-((S)-(1-ethyl-1H-pyrazole-5-carboxamido)((1R,4S)-4-methylcyclohexyl)methyl)imidazo[1,2-b][1,2,4]triazin-2-yl)methyl)-2-oxo-5-(trifluoromethyl)piperidine-3-carboxylate (60.0 mg, 99.2 μmol, 73.7% yield) as a yellow oil. LCMS [M+H]⁺=605.3 m/z.

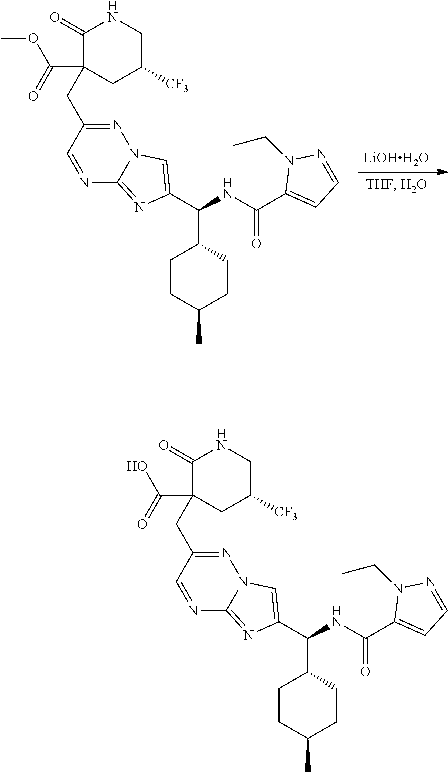

To a solution of methyl (5R)-3-((6-((S)-(1-ethyl-1H-pyrazole-5-carboxamido)((1R,4S)-4-methylcyclohexyl)methyl)imidazo[1,2-b][1,2,4]triazin-2-yl)methyl)-2-oxo-5-(trifluoromethyl)piperidine-3-carboxylate (60.0 mg, 99.2 μmol, 1.00 eq) in THF (1.00 mL) and water (0.10 mL) was added lithium hydroxide monohydrate (16.7 mg, 397 μmol, 4.00 eq). The reaction mixture was stirred at 25° C. for 3 h. The reaction mixture was then adjusted to pH=4 with a 1.0 M aqueous solution of HCl. The resulting solution was concentrated under reduced pressure to afford (5R)-3-((6-((S)-(1-ethyl-1H-pyrazole-5-carboxamido)((1R,4S)-4-methylcyclohexyl)methyl)imidazo[1,2-b][1,2,4]triazin-2-yl)methyl)-2-oxo-5-(trifluoromethyl)piperidine-3-carboxylic acid (58.0 mg, 98.2 μmol, 99.0% yield) as a yellow oil. LCMS [M+H]⁺=591.2 m/z.

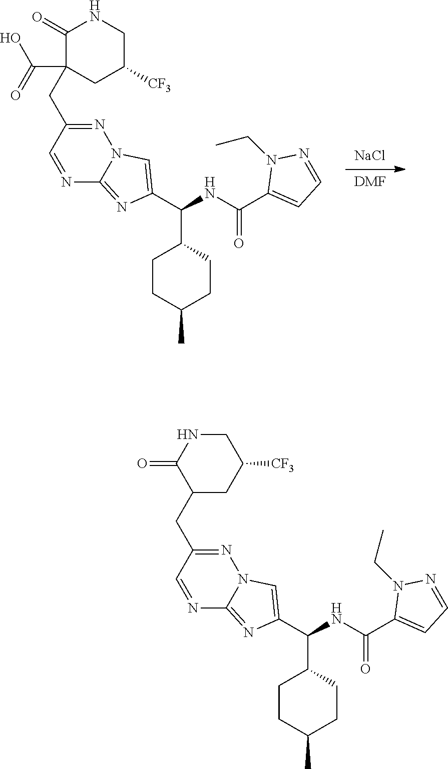

To a solution of (5R)-3-((6-((S)-(1-ethyl-1H-pyrazole-5-carboxamido)((1R,4S)-4-methylcyclohexyl)methyl)imidazo[1,2-b][1,2,4]triazin-2-yl)methyl)-2-oxo-5-(trifluoromethyl)piperidine-3-carboxylic acid (58.0 mg, 98.2 μmol, 1.00 eq) in N,N-dimethylformamide (1.00 mL) was added sodium chloride (23.0 mg, 393 μmol, 4.00 eq). The reaction mixture was stirred at 110° C. for 1 h. The reaction mixture was cooled to RT and diluted with water. An extraction was performed with DCM/MeOH (10/1), and the combined organic layers were washed with brine, dried over anhydrous sodium sulfate, filtered, and concentrated under reduced pressure to give a residue, which was purified by prep-TLC (SiO₂, DCM/MeOH) to afford 1-ethyl-N-((1S)-((1R,4S)-4-methylcyclohexyl)(2-(((5R)-2-oxo-5-(trifluoromethyl)piperidin-3-yl)methyl)imidazo[1,2-b][1,2,4]triazin-6-yl)methyl)-1H-pyrazole-5-carboxamide (26.0 mg, 47.6 μmol, 48.4% yield) as a yellow oil. LCMS [M+H]⁺=547.3 m/z. Compound 2 was isolated as the second eluting, single stereoisomer by chiral SFC purification (column: Daicel Chiralpak IC (250 mm×30 mm, 10 um); mobile phase: [CO₂-0.1% NH₃·H₂O in EtOH]; B %: 50%) (12.66 mg, 21.2 μmol, 44.5% yield) as a yellow solid. LCMS [M+H]⁺=547.3 m/z. Compound 1 was isolated as the first eluting, single stereoisomer by chiral SFC purification.

Example 10: Preparation of (5R)-3-((6-((S))-amino(4,4-difluoroyclohexyl) methyl)imidazo[1,2-b][1,2,4]triazin-2-yl)methyl)-5-(trifluoromethyl)piperidin-2-one (Intermediate 1)

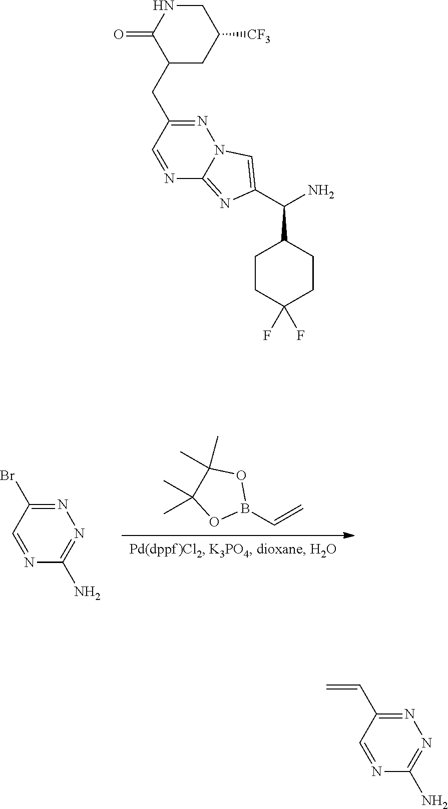

To a solution of 6-bromo-1,2,4-triazin-3-amine (10.0 g, 57.2 mmol, 1.00 eq) and 4,4,5,5-tetramethyl-2-vinyl-1,3,2-dioxaborolane (9.68 g, 62.9 mmol, 10.7 mL, 1.10 eq) in dioxane (100 mL) and water (20 mL) was added successively [1,1′-bis(diphenylphosphino)ferrocene]dichloropalladium(II) (3.35 g, 4.57 mmol, 0.0800 eq) and potassium phosphate tribasic (24.3 g, 114 mmol, 2.00 eq). The reaction mixture was degassed and purged with nitrogen (3 cycles), and then stirred at 80° C. for 2 h under nitrogen atmosphere. The reaction mixture was cooled to RT and concentrated under reduced pressure to give a residue, which was purified by column chromatography (SiO₂, petroleum ether/EtOAc) to afford 6-vinyl-1,2,4-triazin-3-amine (4.50 g, 32.0 mmol, 56.0% yield) as a yellow solid. LCMS [M+H]⁺=123.0 m/z.

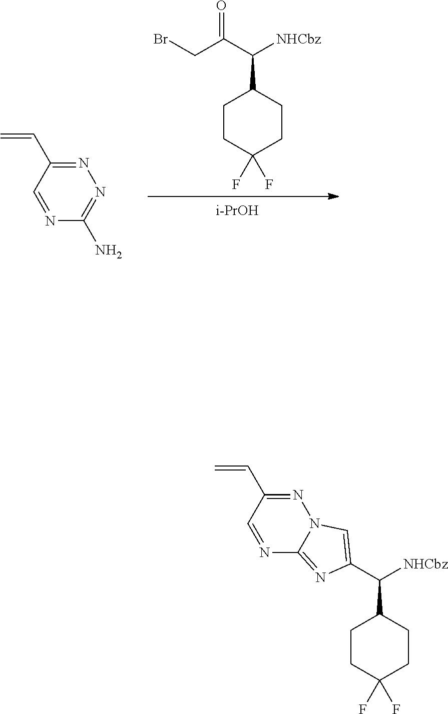

To a solution of benzyl (S)-(3-bromo-1-(4,4-difluorocyclohexyl)-2-oxopropyl)carbamate (1.50 g, 3.71 mmol, 1.00 eq) in isopropanol (20.0 mL) was added 6-vinyl-1,2,4-triazin-3-amine (679 mg, 4.82 mmol, 1.30 eq). The reaction mixture was stirred at 90° C. for 2 h. The reaction mixture was cooled to RT and concentrated under reduced pressure to give a residue, which was purified by column chromatography (SiO₂, petroleum ether/EtOAc) to afford benzyl (S)-((4,4-difluorocyclohexyl)(2-vinylimidazo[1,2-b][1,2,4]triazin-6-yl)methyl)carbamate (1.40 g, 3.28 mmol, 88.3% yield) as a yellow oil. ¹H NMR (400 MHz, CDCl₃) d 8.65 (s, 1H), 7.78 (s, 1H), 7.35-7.39 (m, 5H), 6.83 (dd, J₁=18.0 Hz, J₂=11.6 Hz, 1H), 6.31 (d, J=18.0 Hz, 1H), 5.82 (d, J=11.2 Hz, 1H), 5.75 (d, J=8.4 Hz, 1H), 5.10-5.13 (m, 2H), 2.10-2.15 (m, 2H), 1.94-1.98 (m, 1H), 1.75-1.81 (m, 2H), 1.62-1.66 (m, 2H), 1.37-1.46 (m, 2H). LCMS [M+H]⁺=428.2 m/z.

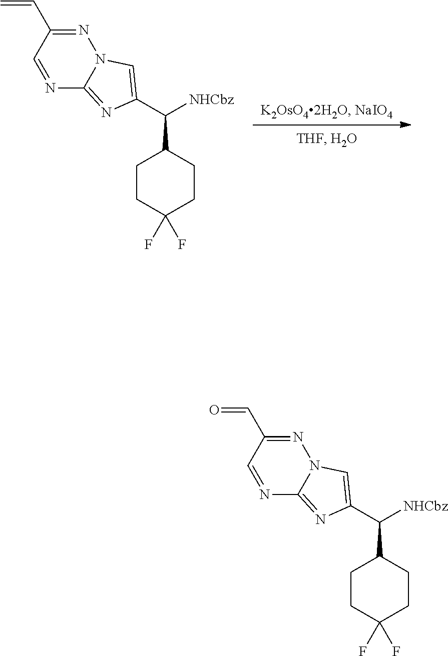

To a solution of benzyl (S)-((4,4-difluorocyclohexyl)(2-vinylimidazo[1,2-b][1,2,4]triazin-6-yl)methyl)carbamate (900 mg, 2.11 mmol, 1.00 eq) in THF (10 mL) and water (20 mL) was added sodium periodate (1.80 g, 8.42 mmol, 467 μL, 4.00 eq) followed by potassium osmate(IV) dihydrate (155 mg, 421 μmol, 0.200 eq). The reaction mixture was stirred at 25° C. for 2 h. The reaction mixture was then diluted with water and extracted with EtOAc. The combined organic layers were washed with brine, dried over anhydrous sodium sulfate, filtered, and concentrated under reduced pressure to give a residue, which was purified by column chromatography (SiO₂, petroleum ether/EtOAc) to afford benzyl (S)-((4,4-difluorocyclohexyl)(2-formylimidazo[1,2-b][1,2,4]triazin-6-yl)methyl)carbamate (390 mg, 908 μmol, 43.1% yield) as a yellow oil. ¹H NMR (400 MHz, CDCl₃) d 10.10 (s, 1H), 9.01 (s, 1H), 7.99 (s, 1H), 7.33-7.37 (m, 5H), 5.67 (d, J=9.6 Hz, 1H), 5.08-5.13 (m, 2H), 4.97 (t, J=8.0 Hz, 1H), 2.11-2.20 (m, 2H), 1.93-2.00 (m, 1H), 1.62-1.79 (m, 4H), 1.37-1.50 (m, 2H).

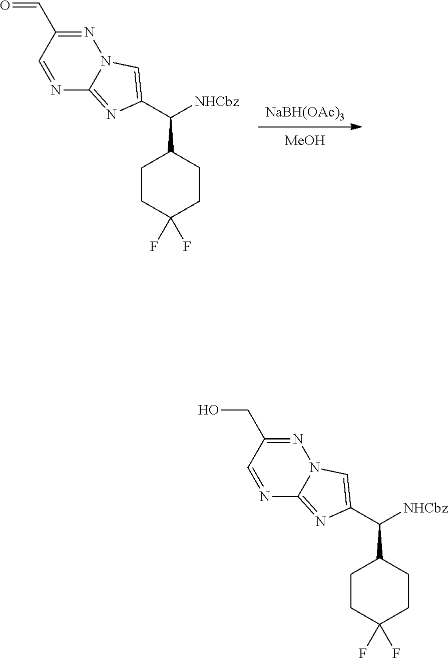 [SMILES: "MeOH" ; O=Cc1cnc2nc([C@@H](NC(=O)OCc3ccccc3)C3CCC(F)(F)CC3)cn2n1 ; O=C(N[C@H](c1cn2nc(CO)cnc2n1)C1CCC(F)(F)CC1)OCc1ccccc1 ; "NaBH(OAc)3"]

To a solution of benzyl (S)-((4,4-difluorocyclohexyl)(2-formylimidazo[1,2-b][1,2,4]triazin-6-yl)methyl)carbamate (390 mg, 908 μmol, 1.00 eq) in MeOH (6.00 mL) was added sodium triacetoxyborohydride (577 mg, 2.72 mmol, 3.00 eq). The reaction mixture was stirred at 25° C. for 3 h. The reaction mixture was diluted with water and extracted with a mixture of DCM and MeOH (10/1). The combined organic layers were washed with brine, dried over anhydrous sodium sulfate, filtered, and concentrated under reduced pressure to give a residue, which was purified by prep-TLC (SiO₂, DCM/MeOH) to afford benzyl (S)-((4,4-difluorocyclohexyl)(2-(hydroxymethyl)imidazo[1,2-b][1,2,4]triazin-6-yl)methyl)carbamate (180 mg, 417 μmol, 45.9% yield) as a yellow solid. LCMS [M+H]⁺=432.2 m/z.

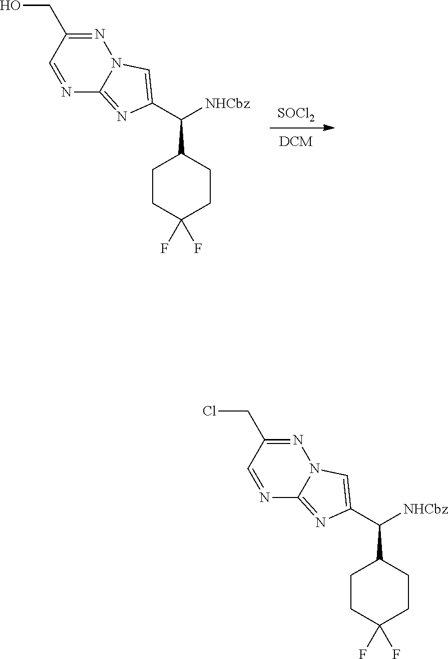

To a solution of benzyl (S)-((4,4-difluorocyclohexyl)(2-(hydroxymethyl)imidazo[1,2-b][1,2,4]triazin-6-yl)methyl)carbamate (180 mg, 417 μmol, 1.00 eq) in DCM (4.00 mL) was added thionyl chloride (149 mg, 1.25 mmol, 90.9 μL, 3.00 eq) at 0° C. The reaction mixture was stirred at 25° C. for 1 h. The reaction mixture was concentrated under reduced pressure to afford benzyl (S)-((2-(chloromethyl)imidazo[1,2-b][1,2,4]triazin-6-yl)(4,4-difluorocyclohexyl)methyl)carbamate (185 mg, crude) as a yellow solid. LCMS [M+H]⁺=450.2 m/z.

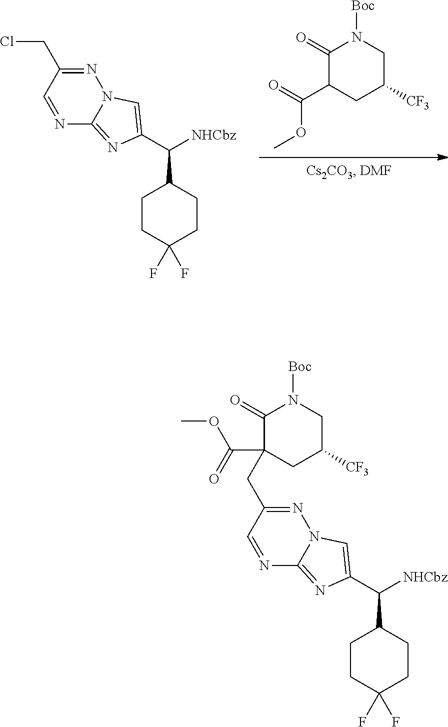

To a solution of 1-(tert-butyl) 3-methyl (5R)-2-oxo-5-(trifluoromethyl)piperidine-1,3-dicarboxylate (201 mg, 617 μmol, 1.50 eq) in N,N-dimethylformamide (5.00 mL) was added cesium carbonate (402 mg, 1.23 mmol, 3.00 eq). The reaction mixture was stirred at 25° C. for 30 min before adding benzyl (S)-((2-(chloromethyl)imidazo[1,2-b][1,2,4]triazin-6-yl)(4,4-difluorocyclohexyl)methyl)carbamate (185 mg, 411 μmol, 1.00 eq). The reaction mixture was stirred at 40° C. for 2 h. The reaction mixture was diluted with water and extracted with EtOAc. The combined organic layers were washed with brine, dried over anhydrous sodium sulfate, filtered, and concentrated under reduced pressure to give a residue, which was purified by prep-TLC (SiO₂, petroleum ether/EtOAc) to afford 1-(tert-butyl) 3-methyl (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-1,3-dicarboxylate (200 mg, 271 μmol, 65.8% yield) as a yellow solid. LCMS [M+H]⁺=739.3 m/z.

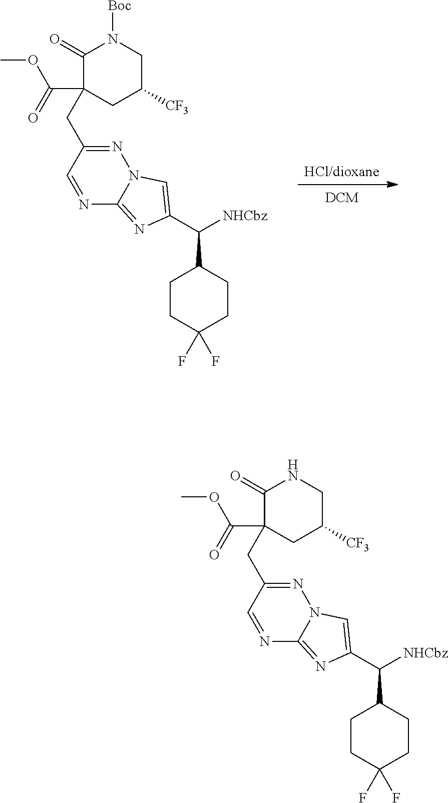

To a solution of 1-(tert-butyl) 3-methyl (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-1,3-dicarboxylate (200 mg, 271 μmol, 1.00 eq) in DCM (2.00 mL) was added a solution of HCl in dioxane (4.00 M, 0.500 mL, 7.39 eq). The reaction mixture was stirred at 25° C. for 2 h. The reaction mixture was concentrated under reduced pressure to afford methyl (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylate (180 mg, crude) as a yellow oil. LCMS [M+H]⁺=639.3 m/z.

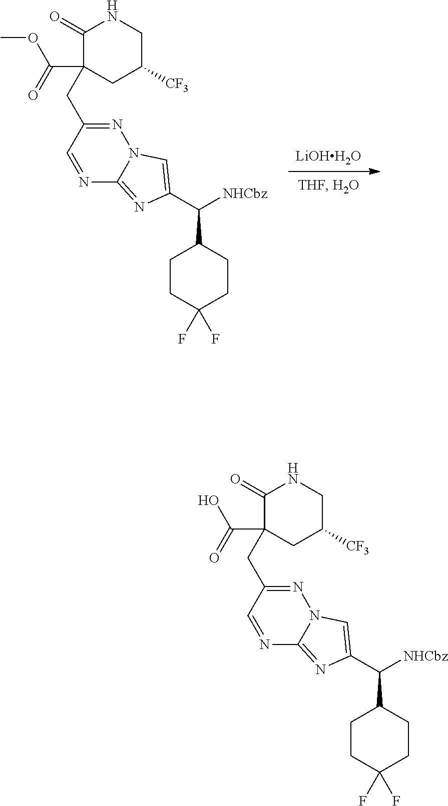

To a solution of methyl (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylate (180 mg, 267 μmol, 1.00 eq) in THF (3.00 mL) and water (0.50 mL) was added lithium hydroxide monohydrate (55.9 mg, 1.33 mmol, 5.00 eq). The reaction mixture was stirred at 25° C. for 2 h. The reaction mixture was then adjusted to pH=4 with a 1.0 M aqueous solution of HCl. The resulting solution was concentrated under reduced pressure to afford (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylic acid (165 mg, 264 μmol, 99.1% yield) as a yellow oil. LCMS [M+H]⁺=625.3 m/z.

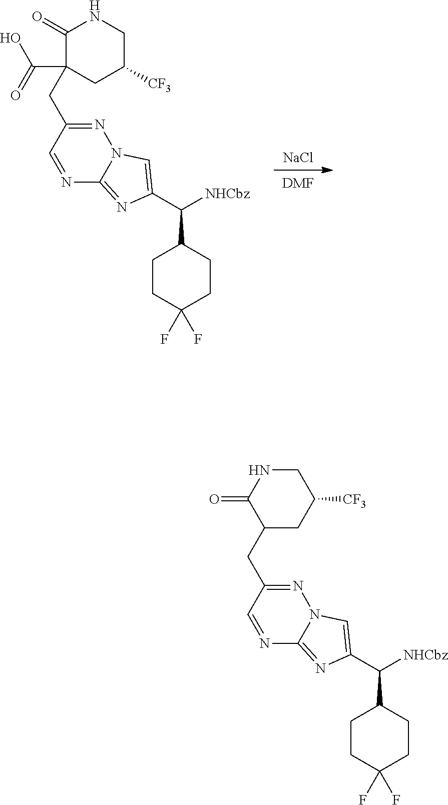

To a solution of (5R)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylic acid (165 mg, 264 μmol, 1.00 eq) in N,N-dimethylformamide (3.00 mL) was added sodium chloride (77.2 mg, 1.32 mmol, 5.00 eq). The reaction mixture was stirred at 110° C. for 1 h. The reaction mixture was cooled to RT and diluted with water. An extraction was performed with EtOAc, the combined organic layers were washed with brine, dried over anhydrous sodium sulfate, filtered, and concentrated under reduced pressure to give a residue, which was purified by prep-TLC (SiO₂, DCM/MeOH) to afford benzyl ((1S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)carbamate (125 mg, 215 μmol, 81.5% yield) as a yellow oil. LCMS [M+H]⁺=581.2 m/z. The desired isomer and title compound was isolated as the first eluting, single stereoisomer by chiral SFC purification (column: Daicel Chiralpak IG (250 mm×30 mm, 10 um); mobile phase: [CO₂-0.1% NH₃·H₂O in EtOH]; B %: 60%, isocratic elution mode); (65.0 mg, 112 μmol, 90.3% yield) as a yellow solid. ¹H NMR (400 MHz, CDCl₃) d 8.41 (s, 1H), 7.76 (s, 1H), 7.30-7.39 (m, 5H), 5.96 (s, 1H), 5.81 (d, J=9.2 Hz, 1H), 5.05-5.16 (m, 2H), 4.87 (t, J=8.0 Hz, 1H), 3.55-3.64 (m, 1H), 3.38-3.54 (m, 2H), 3.14 (dd, J₁=15.2 Hz, J₂=7.2 Hz, 1H), 2.94-3.02 (m, 1H), 2.68-2.85 (m, 1H), 2.26-2.36 (m, 1H), 2.09-2.18 (m, 2H), 1.92-2.01 (m, 1H), 1.71-1.77 (m, 1H), 1.59-1.69 (m, 4H), 1.33-1.50 (m, 2H). LCMS [M+H]⁺=581.2 m/z.

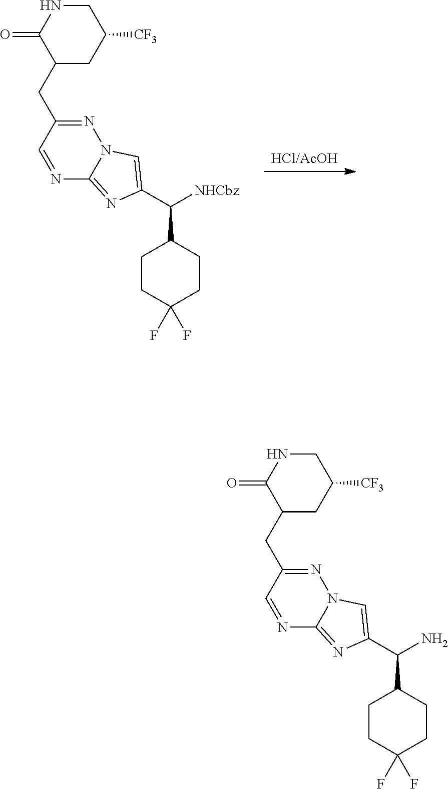 [SMILES: O=C(N[C@H](c1cn2nc(CC3C[C@@H](C(F)(F)F)CNC3=O)cnc2n1)C1CCC(F)(F)CC1)OCc1ccccc1 ; "HCl/AcOH" ; N[C@H](c1cn2nc(CC3C[C@@H](C(F)(F)F)CNC3=O)cnc2n1)C1CCC(F)(F)CC1]

To a solution of benzyl ((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)carbamate (65.0 mg, 112 μmol, 1.00 eq) in HCl (12.0 M, 1.00 mL, 107 eq) was added acetic acid (0.100 mL). The reaction mixture was stirred at 60° C. for 30 min. The reaction mixture was cooled to RT and diluted with water. An extraction was performed with DCM. The aqueous layer was adjusted to pH=8 with a saturated aqueous solution of sodium hydrogen carbonate and was extracted with a mixture of DCM/MeOH (10/1). The combined organic layers were dried over anhydrous sodium sulfate, filtered and concentrated under reduced pressure to afford (5R)-3-((6-((S)-amino(4,4-difluorocyclohexyl)methyl)imidazo[1,2-b][1,2,4]triazin-2-yl)methyl)-5-(trifluoromethyl)piperidin-2-one (Intermediate 1, 30.0 mg, 67.2 μmol, 60.0% yield) as a light yellow solid. LCMS [M+H]⁺=447.2 m/z.

### Example 11: Preparation of (5S)-3-((6-((S)-amino(4,4-difluorocyclohexyl)methyl)imidazo[1,2-b][1,2,4]triazin-2-yl)methyl)-5-(trifluoromethyl)piperidin-2-one (Intermediate 2)

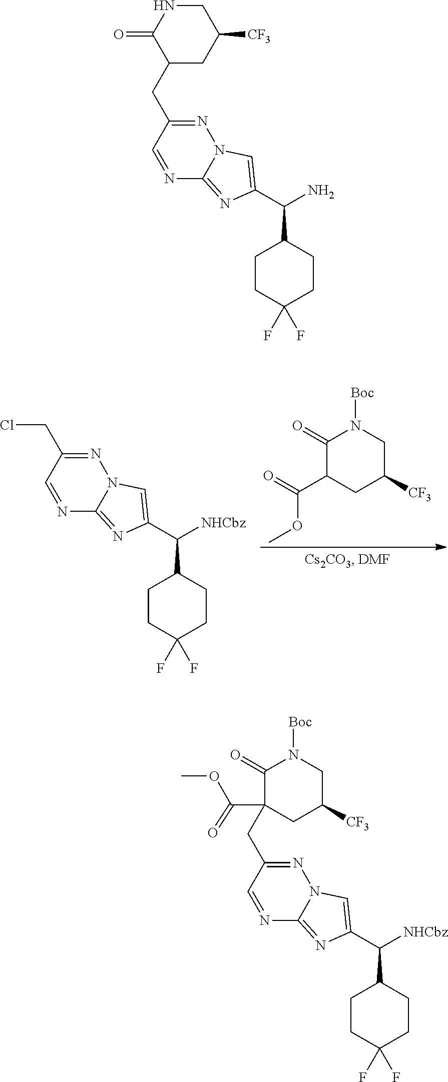 [SMILES: N[C@H](c1cn2nc(CC3C[C@H](C(F)(F)F)CNC3=O)cnc2n1)C1CCC(F)(F)CC1 ; O=C(N[C@H](c1cn2nc(CCl)cnc2n1)C1CCC(F)(F)CC1)OCc1ccccc1 ; COC(=O)C1C[C@H](C(F)(F)F)CN(C(=O)OC(C)(C)C)C1=O ; COC(=O)C1(Cc2cnc3nc([C@@H](NC(=O)OCc4ccccc4)C4CCC(F)(F)CC4)cn3n2)C[C@H](C(F)(F)F)CN(C(=O)OC(C)(C)C)C1=O]

To a solution of 1-(tert-butyl) 3-methyl (5S)-2-oxo-5-(trifluoromethyl)piperidine-1,3-dicarboxylate (2.45 g, 7.54 mmol, 1.30 eq) in N,N-dimethylformamide (26.0 mL) was added cesium carbonate (5.67 g, 17.4 mmol, 3.00 eq). The reaction mixture was stirred at 25° C. for 1 h prior to the addition of benzyl (S)-((2-(chloromethyl)imidazo[1,2-b][1,2,4]triazin-6-yl)(4,4-difluorocyclohexyl)methyl)carbamate (2.61 g, 5.80 mmol, 1.00 eq). The reaction mixture was further stirred at 40° C. for 2 h. The reaction mixture was diluted with water and extracted with DCM. The combined organic layers were washed with brine, dried over anhydrous sodium sulfate, filtered, and concentrated under reduced pressure to give a residue, which was purified by column chromatography (SiO₂, petroleum ether/EtOAc) to afford 1-(tert-butyl) 3-methyl (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-1,3-dicarboxylate (3.30 g, crude) as a white solid. LCMS [M+H]⁺=739.6 m/z.

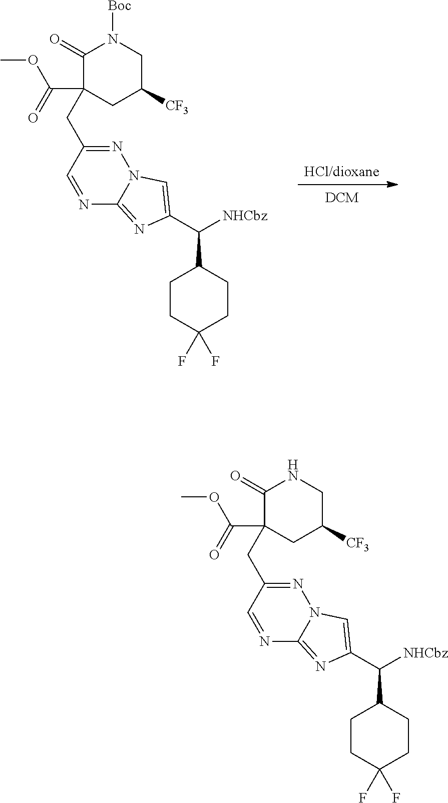

To a solution of 1-(tert-butyl) 3-methyl (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-1,3-dicarboxylate (3.30 g, 4.47 mmol, 1.00 eq) in DCM (26.0 mL) was added a solution of HCl in dioxane (4.00 M, 12.6 mL, 11.3 eq). The reaction mixture was stirred at 25° C. for 1 h. The reaction mixture was filtered and concentrated under reduced pressure to afford methyl (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylate (2.85 g, crude) as a white solid. LCMS [M+H]⁺=639.3 m/z.

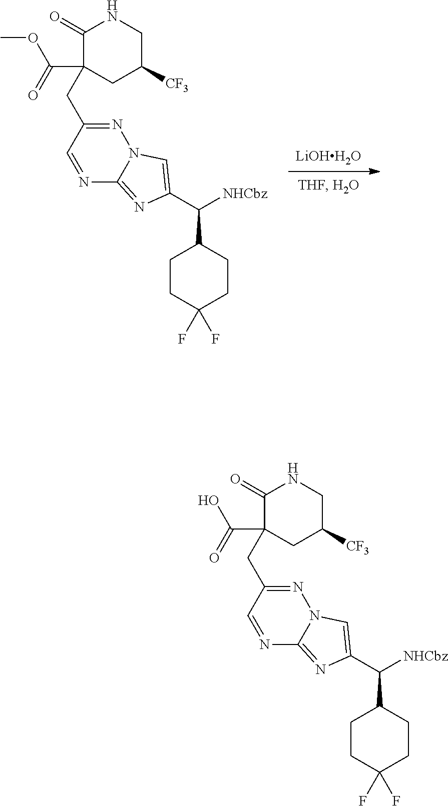

To a solution of methyl (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylate (2.85 g, 4.46 mmol, 1.00 eq) in THF (30.0 mL) and water (5.00 mL) was added lithium hydroxide monohydrate (561 mg, 13.3 mmol, 3.00 eq). The reaction mixture was stirred at 25° C. for 2 h. The combined reaction mixture was extracted with EtOAc. The pH of the aqueous phase was adjusted to 3 with a 1.0 M aqueous solution of HCl and extracted with EtOAc. The combined organic layers were washed with brine, dried over anhydrous sodium sulfate, filtered, and concentrated under reduced pressure to afford (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylic acid (2.79 g, crude) as a white solid. LCMS [M+H]⁺=625.2 m/z.

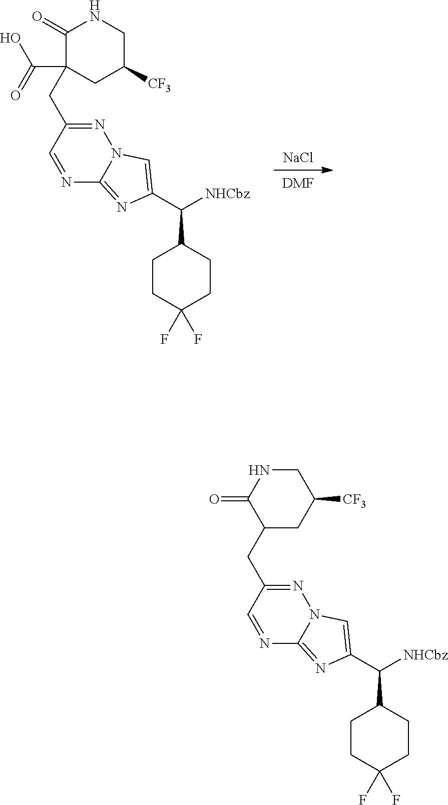

To a solution of (5S)-3-((6-((S)-(((benzyloxy)carbonyl)amino)(4,4-difluorocyclohexyl)methyl)imidazo[1,2-b][1,2,4]triazin-2-yl)methyl)-2-oxo-5-(trifluoromethyl)piperidine-3-carboxylic acid (2.79 g, 4.47 mmol, 1.00 eq) in N,N-dimethylformamide (26.0 mL) was added sodium chloride (1.31 g, 22.3 mmol, 5.00 eq). The reaction mixture was stirred at 110° C. for 1 h. The reaction mixture was cooled to RT and diluted with water. An extraction was performed with DCM. The combined organic layers were washed with brine, dried over anhydrous sodium sulfate, filtered, and concentrated under reduced pressure to afford benzyl ((1S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)carbamate. The desired stereoisomer and title compound was isolated as the first eluting, single stereoisomer by chiral SFC purification (column: Daicel Chiralpak IG (250 mm×30 mm, 10 um); mobile phase: [CO₂-0.1% NH₃·H₂O in EtOH]; B %: 50%, isocratic elution mode); (0.60 g, 1.03 mmol, 23.0% yield) as a white solid. ¹H NMR (400 MHz, CDCl₃) d 8.43 (s, 1H), 7.75 (s, 1H), 7.21-7.32 (m, 5H), 5.93-6.06 (m, 1H), 5.74 (d, J=12.0 Hz, 1H), 4.97-5.15 (m, 2H), 4.79 (t, J=8.0 Hz, 1H), 3.24-3.60 (m, 3H), 2.92-3.09 (m, 2H), 2.54-2.75 (m, 1H), 2.13-2.21 (m, 1H), 1.99-2.08 (m, 2H), 1.80-1.96 (m, 3H), 1.56-1.71 (m, 3H), 1.25-1.43 (m, 2H). LCMS [M+H]⁺=581.2 m/z. The second eluting isomer of this synthesis was used for the synthesis of Compound 5.

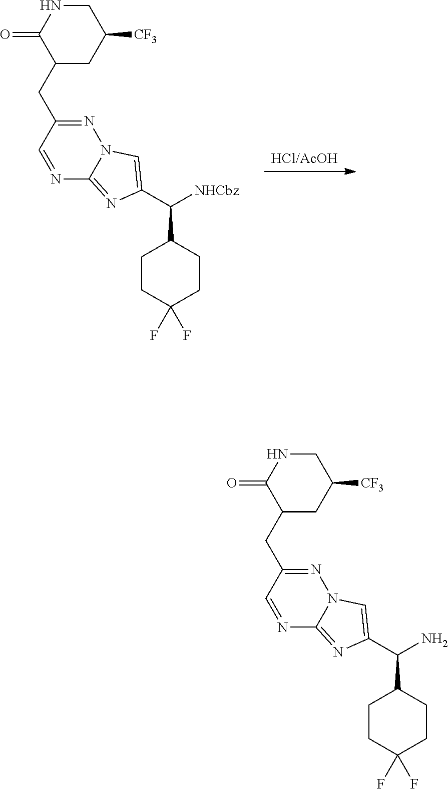 [SMILES: O=C(N[C@H](c1cn2nc(CC3C[C@H](C(F)(F)F)CNC3=O)cnc2n1)C1CCC(F)(F)CC1)OCc1ccccc1 ; "HCl/AcOH" ; N[C@H](c1cn2nc(CC3C[C@H](C(F)(F)F)CNC3=O)cnc2n1)C1CCC(F)(F)CC1]

To a solution of benzyl ((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)carbamate (0.60 g, 1.03 mmol, 1.00 eq) in HCl (12.0 M, 3.75 mL, 43.5 eq) was added acetic acid (1.00 mL). The reaction mixture was stirred at 60° C. for 30 min. The reaction mixture was cooled to RT and diluted with water (30.0 mL). An extraction was performed with EtOAc. The aqueous layer was adjusted to pH=8 with a saturated aqueous solution of sodium hydrogen carbonate and was extracted with EtOAc. The combined organic layers were dried over anhydrous sodium sulfate, filtered, and concentrated under reduced pressure to afford (5S)-3-((6-((S)-amino(4,4-difluorocyclohexyl)methyl)imidazo[1,2-b][1,2,4]triazin-2-yl)methyl)-5-(trifluoromethyl)piperidin-2-one (Intermediate 2, 0.40 g, 0.90 mmol, 87.4% yield) as a white solid. LCMS [M+H]⁺=447.2 m/z.

### General Procedure 1

To a solution of (5R)-3-((6-((S)-amino(4,4-difluorocyclohexyl)methyl)imidazo[1,2-b][1,2,4]triazin-2-yl)methyl)-5-(trifluoromethyl)piperidin-2-one (Intermediate 1) or (5S)-3-((6-((S)-amino(4,4-difluorocyclohexyl)methyl)imidazo[1,2-b][1,2,4]triazin-2-yl)methyl)-5-(trifluoromethyl)piperidin-2-one (Intermediate 2) (1.00 eq) and carboxylic acid in pyridine (2.00 mL) was added EDCI (3.00 eq). The reaction mixture was stirred at 25° C. for 1-2 h. The reaction mixture was diluted with water and extracted with EtOAc. The combined organic layers were dried over anhydrous sodium sulfate, filtered, and concentrated under reduced pressure to give a residue, which was purified by prep-TLC or prep-HPLC to afford the final compound.

### Example 12: Preparation of N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 3)

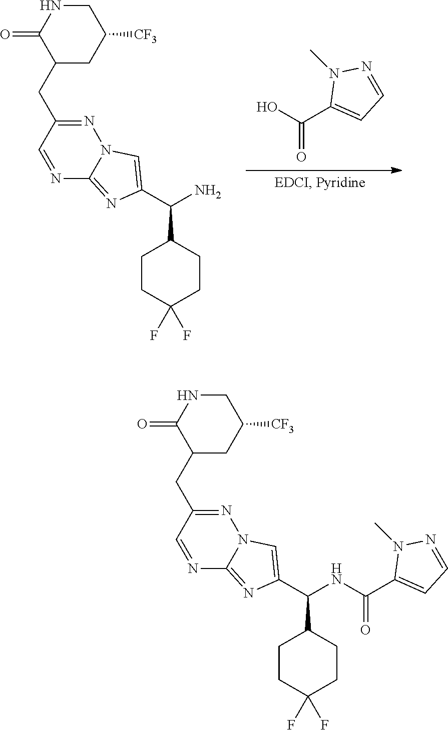

Compound 3 was synthesized following General Procedure 1, employing Intermediate 1 and 1-methyl-1H-pyrazole-5-carboxylic acid (13.5 mg, 107 μmol, 1.20 eq), and purified by prep-HPLC (column: Phenomenex luna C18 150 mm×25 mm, 10 um; mobile phase: [water (FA)—ACN]; B %: 33%-63%) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (28.6 mg, 50.9 μmol, 56.8% yield) as a yellow solid. LCMS [M+H]⁺=555.1 m/z.

### Example 13: Preparation of N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 4 and 5)

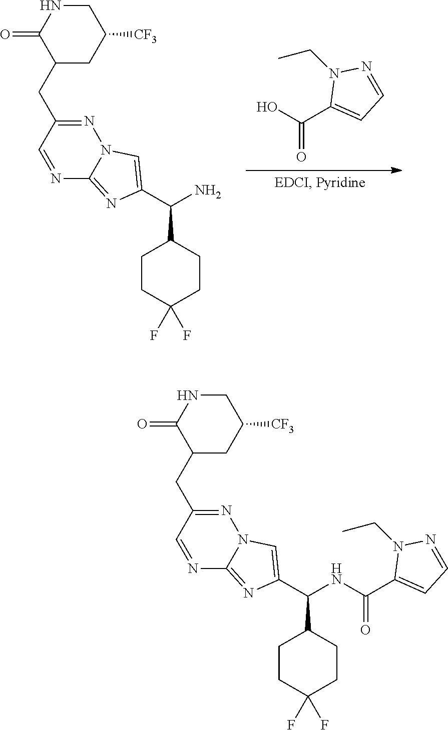

Compound 4 was synthesized following General Procedure 1, employing Intermediate 1 and 1-ethyl-1H-pyrazole-5-carboxylic acid (23.5 mg, 168 μmol, 1.50 eq), and purified by prep-TLC (SiO₂, DCM/MeOH) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (27 mg, 44.45 μmol, 39.7% yield) as a white solid. LCMS [M+H]⁺=569.2 m/z.

Compound 5 was synthesized using an analogous procedure of Compound 4, starting the second eluting isomer of the benzyl ((4,4-difluorocyclohexyl)(2-((2-oxo-5-(trifluoromethyl)piperidin-3-yl)methyl)imidazo[1,2-b][1,2,4]triazin-6-yl)methyl)carbamate synthesis.

### Example 14: Preparation of N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (Compound 6)

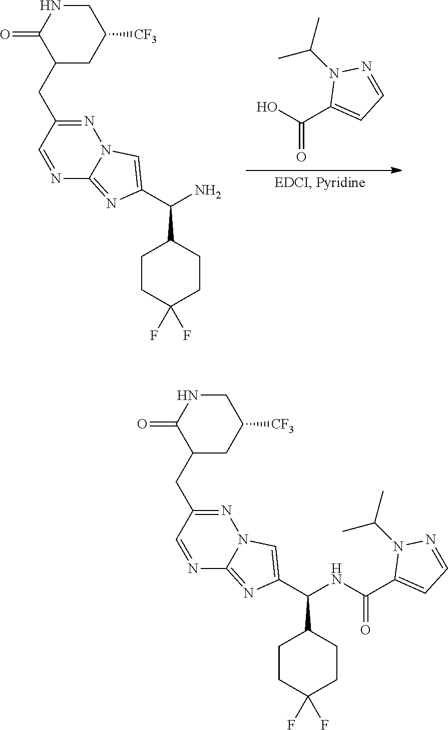

Compound 6 was synthesized following General Procedure 1, employing Intermediate 1 and 1-isopropyl-1H-pyrazole-5-carboxylic acid (9.50 mg, 61.6 μmol, 1.10 eq), and purified by prep-TLC (SiO₂, DCM/MeOH) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (28.5 mg, 47.8 μmol, 85.4% yield) as a white solid. LCMS [M+H]⁺=583.3 m/z.

### Example 15: Preparation of N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-3-(2-fluoroethyl)isoxazole-4-carboxamide (Compound 7)

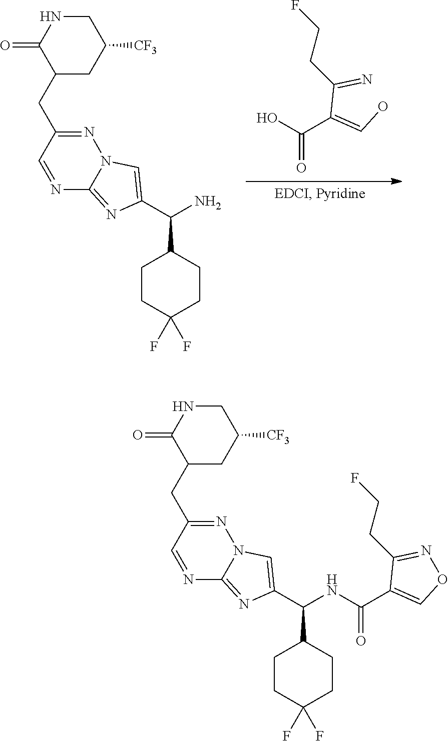

Compound 7 was synthesized following General Procedure 1, employing Intermediate 1 and 3-(2-fluoroethyl)isoxazole-4-carboxylic acid (10.6 mg, 67.2 μmol, 1.00 eq), and purified by Prep-HPLC (column: Waters Xbridge 150 mm×25 mm, 10 um; mobile phase: [water (NH₄HCO₃)—ACN]; gradient: B %: 34%-64%) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-3-(2-fluoroethyl)isoxazole-4-carboxamide (20.6 mg, 34.82 μmol, 51.8% yield) as a white solid. LCMS [M+H]⁺=588.3 m/z.

### Example 16: Preparation of N—((S)-4,4-difluorocyclohexyl)(2-(((5R)-2-oxo-5-(trifluoromethyl)piperidin-3-yl)methyl)imidazo[1,2-b][1,2,4]triazin-6-yl)methyl)-3-isopropylisoxazole-4-carboxamide (Compound 8)

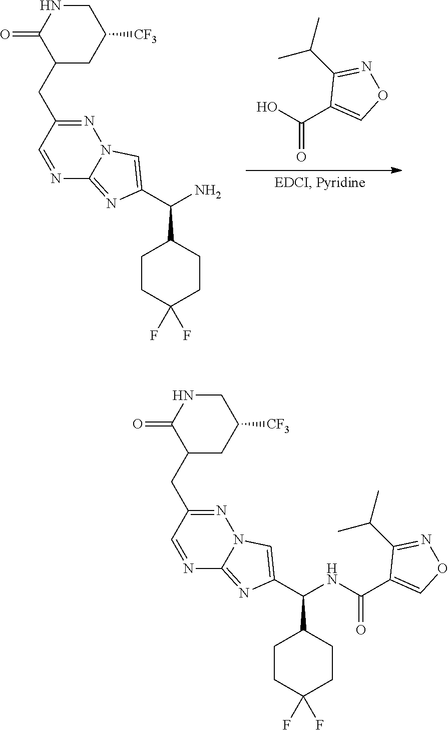

Compound 8 was synthesized following General Procedure 1, employing Intermediate 1 and 3-isopropylisoxazole-4-carboxylic acid (16.6 mg, 107 μmol, 1.20 eq), and purified by prep-HPLC (column: Phenomenex luna C18 150 mm×25 mm, 10 um; mobile phase: [water (FA)—ACN]; B %: 41%-71%) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-3-isopropylisoxazole-4-carboxamide (36.0 mg, 60.0 μmol, 67.0% yield) as a white solid. LCMS [M+H]⁺=584.3 m/z.

### Example 17: Preparation of 3-cyclopropyl-N—((S)-(4,4-difluorocyclohexyl(2-(((5R)-2-oxo-5-(trifluoromethyl)piperidin-3-yl)methyl)imidazo[1,2-b][1,2,4]triazin-6-yl)methyl)isoxazole-4-carboxamide (Compound 9)

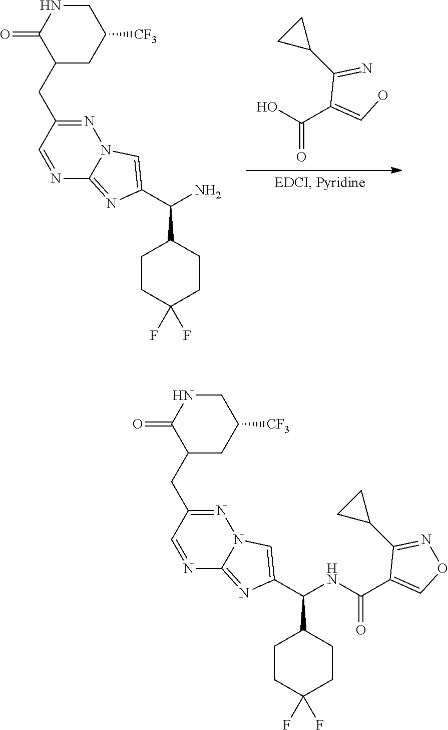

Compound 9 was synthesized following General Procedure 1, employing Intermediate 1 and 3-cyclopropylisoxazole-4-carboxylic acid (14.4 mg, 94.0 μmol, 1.20 eq), and purified by prep-TLC (SiO₂, DCM/MeOH) to afford 3-cyclopropyl-N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)isoxazole-4-carboxamide (15.8 mg, 26.8 μmol, 34.2% yield) as a white solid. LCMS [M+H]⁺=582.3 m/z.

### Example 18: Preparation of N—((S)-(4,4-difluorocyclohexyl)(2-(((5R)?2-oxo-5-(trifluoromethyl)piperidin-3-yl)methyl)imidazo[1,2][1,2,4]triazin-6-yl)methyl-3-(trifluoromethyl)isoxazole-4-carboxamide (Compound 10)

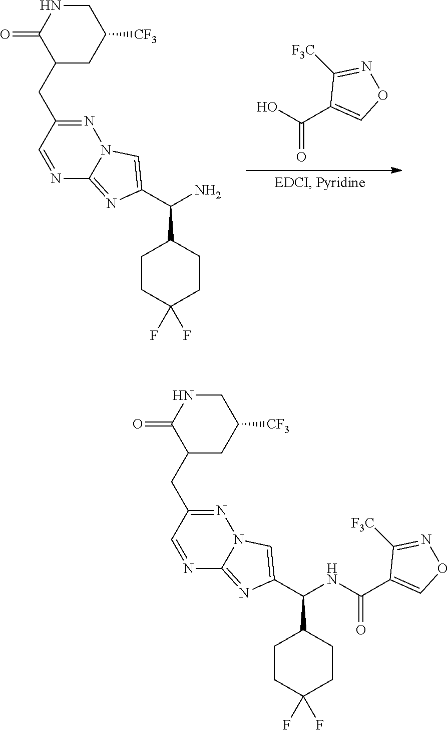

Compound 10 was synthesized following General Procedure 1, employing Intermediate 1 and with 3-(trifluoromethyl)isoxazole-4-carboxylic acid (18.2 mg, 100 μmol, 1.50 eq), and purified by prep-HPLC (column: Phenomenex luna C18 150 mm×25 mm, 10 um; mobile phase: [water (FA)—ACN]; B %: 41%-71%) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-3-(trifluoromethyl)isoxazole-4-carboxamide (27.9 mg, 45.5 μmol, 67.7% yield) as a white solid. LCMS [M+H]⁺=610.1 m/z.

### Example 19: Preparation of N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (Compound 11)

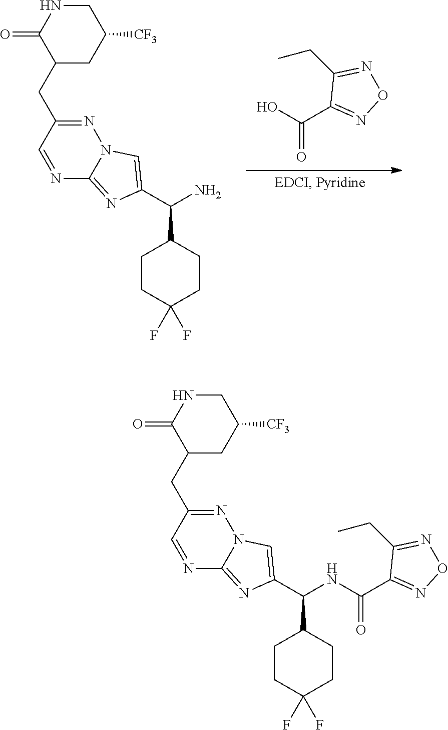

Compound 11 was synthesized following General Procedure 1, employing Intermediate 1 and 4-ethyl-1,2,5-oxadiazole-3-carboxylic acid (15.2 mg, 107 μmol, 1.20 eq), and purified by prep-TLC (SiO₂, DCM/MeOH) to afford N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (28.8 mg, 49.6 μmol, 55.3% yield) as a white solid. LCMS [M+H]⁺=571.3 m/z.

### Example 20: Preparation of N—((S)-(4,4-difluorocyclohexyl(2-(((5R)-2-oxo-5-(trifluoromethyl)piperidin-3-yl)methyl)imidazo[1,2-b][1,2,4]triazin-6-yl)methyl)-4-isopropyl-1,2,5-oxadiazole-3-carboxamide (Compound 12)

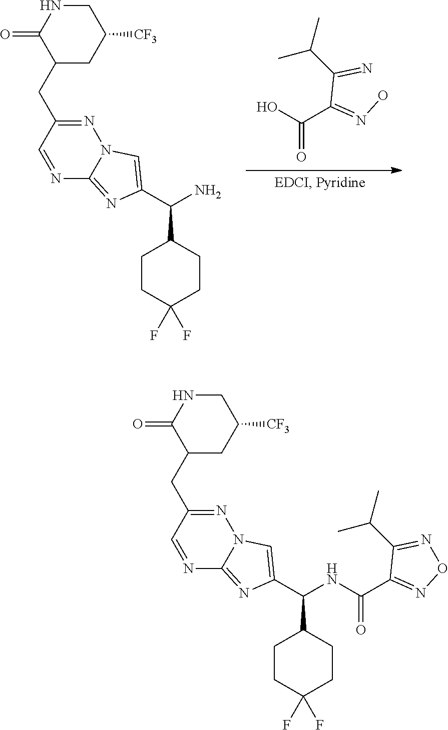

Compound 12 was synthesized following General Procedure 1, employing Intermediate 1 and 4-isopropyl-1,2,5-oxadiazole-3-carboxylic acid (16.7 mg, 107 μmol, 1.20 eq), and purified by prep-HPLC (column: Phenomenex luna C18 150 mm×25 mm, 10 um; mobile phase: [water (FA)—ACN]; B %: 47%-77%) to afford N—((S)-(4,4-difluorocyclohexyl)(2-(((3R)-2-oxo-5-(trifluoromethyl)piperidin-3-yl)methyl)imidazo[1,2-b][1,2,4]triazin-6-yl)methyl)-4-isopropyl-1,2,5-oxadiazole-3-carboxamide (24.98 mg, 42.5 μmol, 47.4% yield, 99.5% purity) as a white solid. LCMS [M+H]⁺=585.2 m/z.

### Example 21: Preparation of 4-cyclopropyl-N—((S)-(4,4-difluorocyclohexyl)(2-(((5R)-2-oxo-5-(trifluoromethyl)piperidin-3-yl)methyl)imidazo[1,2-b][1,2,4]triazin-6-methyl)-1,2,5-oxadiazole-3-carboxamide (Compound 13)

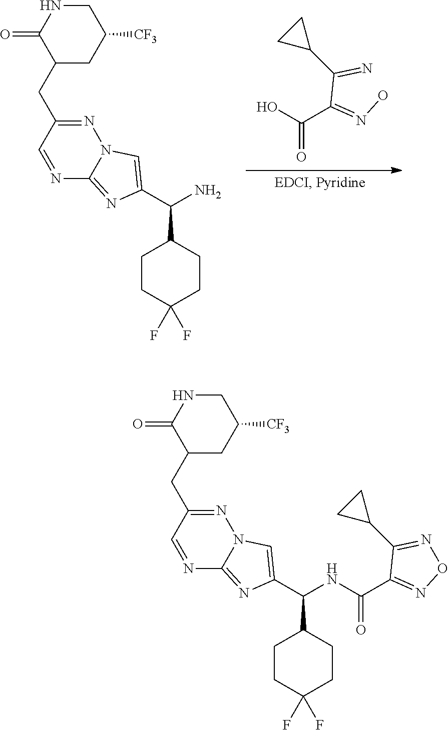

Compound 13 was synthesized following General Procedure 1, employing Intermediate 1 and 4-cyclopropyl-1,2,5-oxadiazole-3-carboxylic acid (34.5 mg, 224 μmol, 2.00 eq), and purified by prep-HPLC (column: Phenomenex luna C18 150 mm×25 mm, 10 um; mobile phase: [water (FA)—ACN]; B %: 42%-72%) to afford 4-cyclopropyl-N—((S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1,2,5-oxadiazole-3-carboxamide (50.5 mg, 84.99 μmol, 75.9% yield) as a white solid. LCMS [M+H]⁺=583.2 m/z.

### Example 22: Preparation of N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 14)

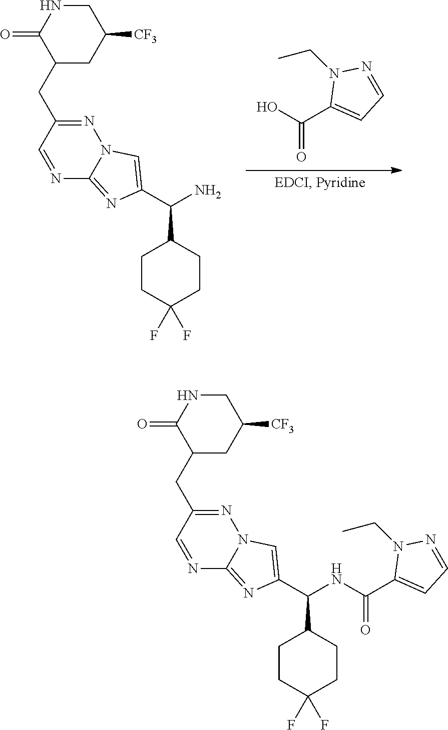

Compound 14 was synthesized following General Procedure 1, employing Intermediate 2 and 1-ethyl-1H-pyrazole-5-carboxylic acid (18.8 mg, 134 μmol, 1.50 eq), and purified by prep-HPLC (column: Phenomenex luna C18 150 mm×25 mm, 10 um; mobile phase: [water (FA)—ACN]; B %: 34%-64%) to afford N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (32.9 mg, 57.5 μmol, 64.2% yield) as a white solid. LCMS [M+H]⁺=569.4 m/z.

### Example 23: Preparation of N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (Compound 15)

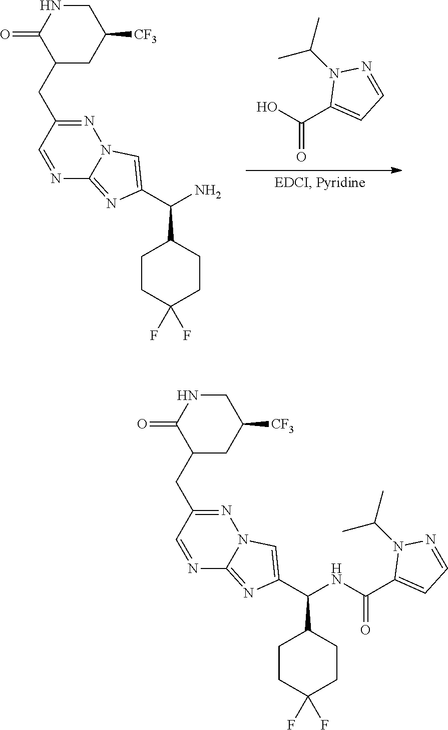

Compound 15 was synthesized following General Procedure 1, employing Intermediate 2 and 1-isopropyl-1H-pyrazole-5-carboxylic acid (20.7 mg, 134 μmol, 1.50 eq), and purified by prep-HPLC (column: Phenomenex luna C18 150 mm×25 mm, 10 um; mobile phase: [water (FA)—ACN]; B %: 36%-66%) to afford N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (33.8 mg, 56.2 μmol, 62.7% yield) as a white solid. LCMS [M+H]⁺=583.4 m/z.

### Example 24: Preparation of 4-cyclopropyl-N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 16)

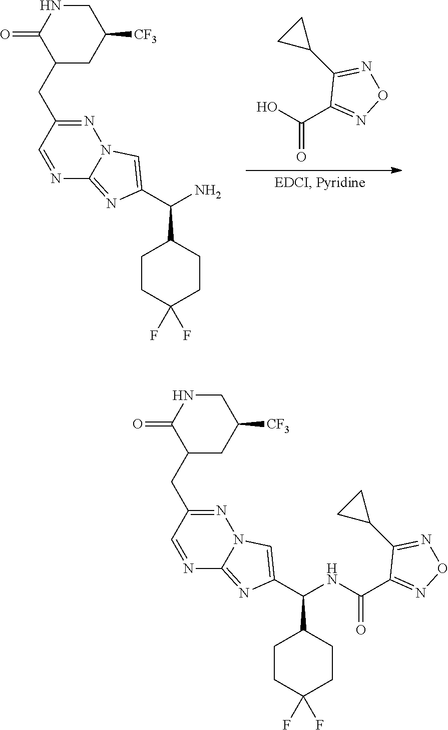

Compound 16 was synthesized following General Procedure 1, employing Intermediate 2 and 4-cyclopropyl-1,2,5-oxadiazole-3-carboxylic acid (20.7 mg, 134 μmol, 1.50 eq) and was purified by prep-HPLC (column: Phenomenex luna C18 150 mm×25 mm, 10 um; mobile phase: [water (FA)—ACN]; B %: 72%, isocratic elution mode) to afford 4-cyclopropyl-N—((S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)-1,2,5-oxadiazole-3-carboxamide (31.5 mg, 54.1 μmol, 60.3% yield) as a white solid. LCMS [M+H]⁺=583.4 m/z.

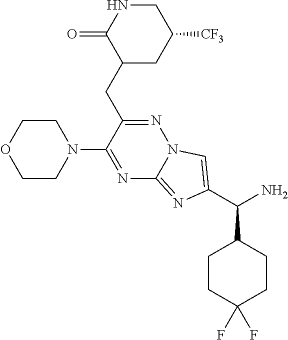 [SMILES: N[C@H](c1cn2nc(CC3C[C@@H](C(F)(F)F)CNC3=O)c(N3CCOCC3)nc2n1)C1CCC(F)(F)CC1]

### (5R)-3-((6-((S)-amino(4,4-difluorocyclohexyl)methyl)-3-morpholinoimidazo[1,2-b][1,2,4]triazin-2-yl)methyl)-5-(trifluoromethyl)piperidin-2-one (Intermediate 4)

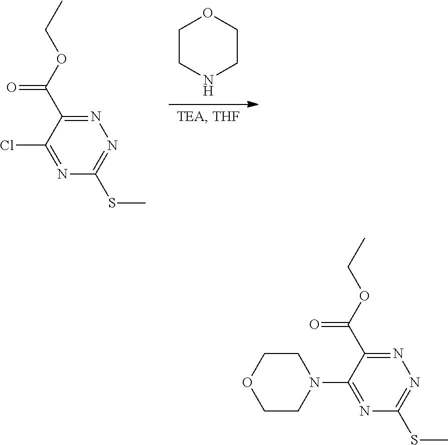

To a solution of ethyl 5-chloro-3-(methylthio)-1,2,4-triazine-6-carboxylate (15.0 g, 64.1 mmol, 1.00 eq) in THF (100 mL) was added triethylamine (9.74 g, 96.2 mmol, 13.4 mL, 1.50 eq) followed by morpholine (6.15 g, 70.6 mmol, 6.21 mL, 1.10 eq). The reaction mixture was stirred at 15° C. for 1 h. The reaction mixture was diluted with water and extracted with EtOAc. The combined organic layers were dried over anhydrous sodium sulfate, filtered, and concentrated under reduced pressure to afford ethyl 3-(methylthio)-5-morpholino-1,2,4-triazine-6-carboxylate (16.0 g, 56.3 mmol, 87.7% yield) as a yellow solid. ¹H NMR (400 MHz, CDCl₃) d 4.36-4.39 (m, 2H), 3.57-3.71 (m, 8H), 2.51-2.52 (m, 3H), 1.16-1.20 (m, 3H). LCMS [M+H]⁺=285.0 m/z.

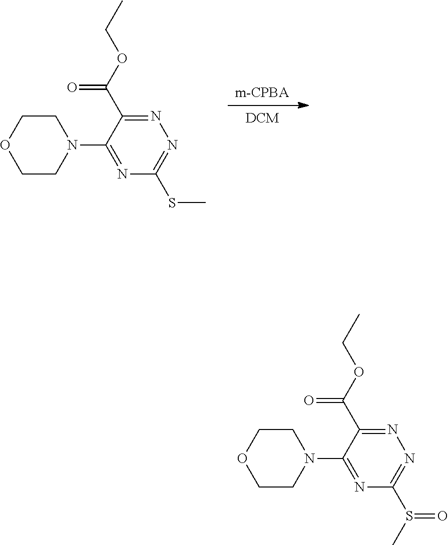

To a solution of ethyl 3-(methylthio)-5-morpholino-1,2,4-triazine-6-carboxylate (3.00 g, 10.5 mmol, 1.00 eq) in DCM (30 mL) was added 3-chloroperbenzoic acid (2.28 g, 10.5 mmol, 80% purity, 1.00 eq) at 0° C. The reaction mixture was stirred at 0° C. for 30 min. The reaction mixture was concentrated under reduced pressure to afford ethyl 3-(methylsulfinyl)-5-morpholino-1,2,4-triazine-6-carboxylate (3.20 g, crude) as a yellow oil. LCMS [M+H]⁺=301.0 m/z.

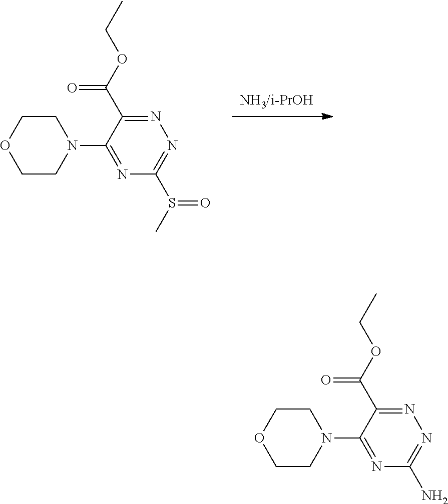 [SMILES: CCOC(=O)c1nnc(S(C)=O)nc1N1CCOCC1 ; "NH3/i-PrOH" ; CCOC(=O)c1nnc(N)nc1N1CCOCC1]

A solution of ethyl 3-(methylsulfinyl)-5-morpholino-1,2,4-triazine-6-carboxylate (3.00 g, 9.99 mmol, 1.00 eq) in ammonia in isopropanol (3.5 M, 11.42 mL, 4.00 eq) was stirred at 0° C. for 15 min. The reaction mixture was filtered and concentrated under reduced pressure to afford ethyl 3-amino-5-morpholino-1,2,4-triazine-6-carboxylate (2.10 g, 8.29 mmol, 83.0% yield) as a yellow solid. LCMS [M+H]⁺=254.0 m/z.

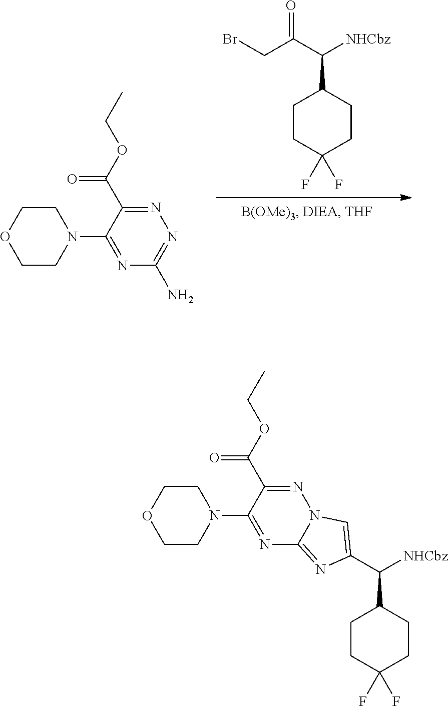

To a solution of ethyl 3-amino-5-morpholino-1,2,4-triazine-6-carboxylate (1.50 g, 5.92 mmol, 1.00 eq) in THF (20.0 mL) was added benzyl (S)-(3-bromo-1-(4,4-difluorocyclohexyl)-2-oxopropyl)carbamate (2.87 g, 7.11 mmol, 1.20 eq), trimethyl borate (2.46 g, 23.6 mmol, 2.68 mL, 4.00 eq), and N,N-diisopropylethylamine (3.06 g, 23.6 mmol, 4.13 mL, 4.00 eq). The reaction mixture was stirred at 70° C. for 1 h. The reaction was cooled to RT. The reaction mixture was filtered and concentrated under reduced pressure to give a residue, which was purified by column chromatography (SiO₂, petroleum ether/EtOAc) to afford ethyl (S)-6-((((benzyloxy)carbonyl)amino)(4,4-difluorocyclohexyl)methyl)-3-morpholinoimidazo[1,2-b][1,2,4]triazine-2-carboxylate (2.40 g, 4.30 mmol, 72.5% yield) as a yellow solid. LCMS [M+H]⁺=559.4 m/z.

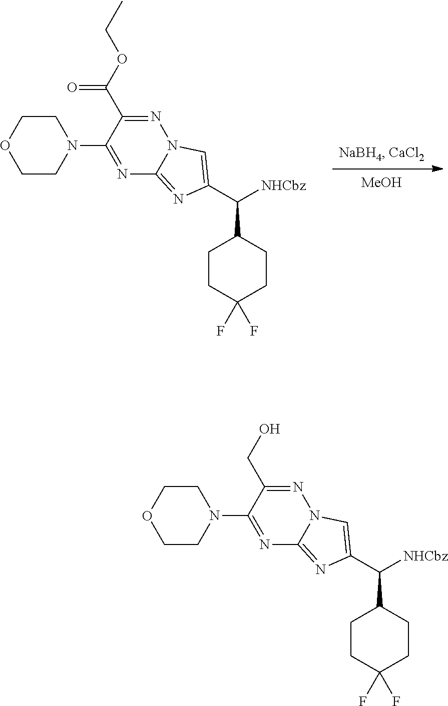

To a solution of ethyl (S)-6-((((benzyloxy)carbonyl)amino)(4,4-difluorocyclohexyl)methyl)-3-morpholinoimidazo[1,2-b][1,2,4]triazine-2-carboxylate (2.20 g, 3.94 mmol, 1.00 eq) in MeOH (15.0 mL) was added sodium borohydride (0.290 g, 7.67 mmol, 1.95 eq) and calcium chloride (437 mg, 3.94 mmol, 1.00 q) at 0° C. The reaction mixture was stirred at 15° C. for 1 h. The reaction mixture was quenched by the addition of a saturated aqueous solution of ammonium chloride at 0° C., and then diluted with water and extracted with EtOAc. The combined organic layers were dried over anhydrous sodium sulfate, filtered, and concentrated under reduced pressure to give a residue, which was purified by column chromatography (SiO₂, petroleum ether/EtOAc) to afford benzyl (S)-((4,4-difluorocyclohexyl)(2-(hydroxymethyl)-3-morpholinoimidazo[1,2-b][1,2,4]triazin-6-yl)methyl)carbamate (0.600 g, 1.16 mmol, 29.5% yield) as a yellow solid. LCMS [M+H]⁺=517.3 m/z.

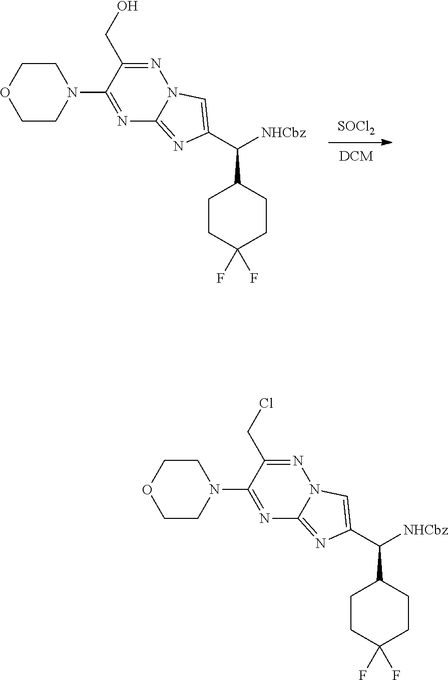

To a solution of benzyl (S)-((4,4-difluorocyclohexyl)(2-(hydroxymethyl)-3-morpholinoimidazo[1,2-b][1,2,4]triazin-6-yl)methyl)carbamate (0.600 g, 1.16 mmol, 1.00 eq) in DCM (5.00 mL) was added thionyl chloride (414 mg, 3.48 mmol, 253 μL, 3.00 eq). The reaction mixture was stirred at 0° C. for 1 h. The reaction mixture was concentrated under reduced pressure to afford benzyl (S)-((2-(chloromethyl)-3-morpholinoimidazo[1,2-b][1,2,4]triazin-6-yl)(4,4-difluorocyclohexyl)methyl)carbamate (0.600 g, 1.12 mmol, 96.5% yield) as a yellow solid.

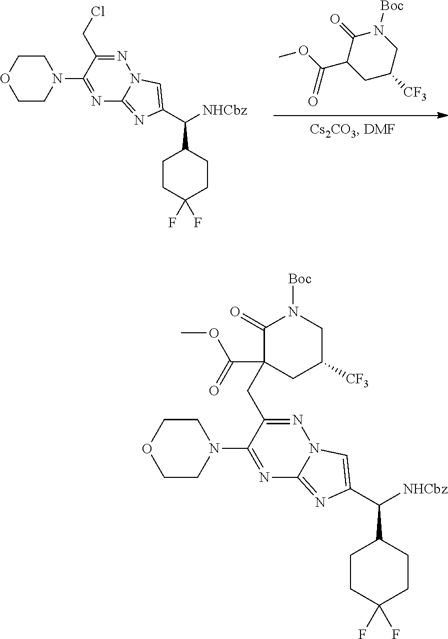

To a solution of benzyl (S)-((2-(chloromethyl)-3-morpholinoimidazo[1,2-b][1,2,4]triazin-6-yl)(4,4-difluorocyclohexyl)methyl)carbamate (250 mg, 467 μmol, 1.00 eq) and 1-(tert-butyl) 3-methyl (5R)-2-oxo-5-(trifluoromethyl)piperidine-1,3-dicarboxylate (228 mg, 701 μmol, 1.50 eq) in N,N-dimethylformamide (4.00 mL) was added cesium carbonate (456 mg, 1.40 mmol, 3.00 eq). The reaction mixture was stirred at 25° C. for 12 h. The reaction mixture was concentrated under reduced pressure to give a residue, which was diluted with water and extracted with EtOAc. The combined organic layers were dried over anhydrous sodium sulfate, filtered, and concentrated under reduced pressure to give a residue, which was purified by column chromatography (SiO₂, petroleum ether/EtOAc) to afford 1-(tert-butyl) 3-methyl (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-1,3-dicarboxylate as a yellow solid. LCMS [M+H]⁺=824.3 m/z.

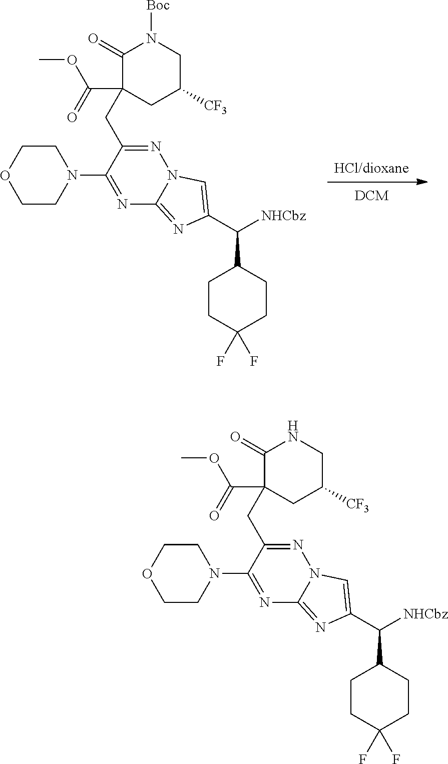

To a solution of 1-(tert-butyl) 3-methyl (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-1,3-dicarboxylate (250 mg, 303 μmol, 1.00 eq) in DCM (3.00 mL) was added a solution of HCl in dioxane (4.00 M, 2.65 mL, 35.0 eq) at 0° C. The reaction mixture was stirred at 25° C. for 1 h. The reaction mixture was concentrated under reduced pressure to afford methyl (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-3-carboxylate as a yellow solid. LCMS [M+H]⁺=724.3 m/z.

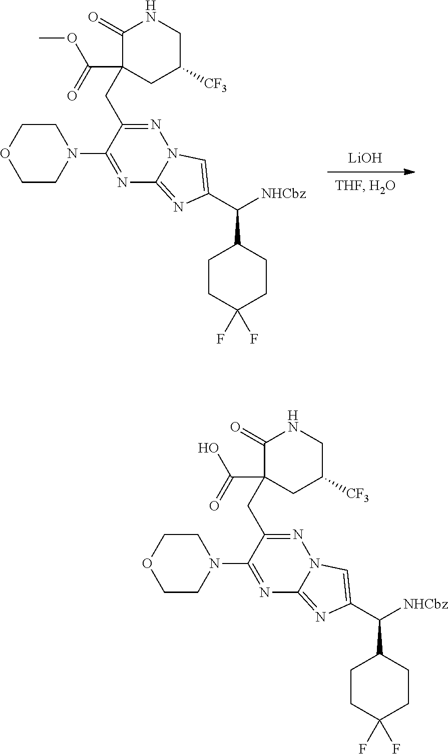

To a solution of methyl (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-3-carboxylate (0.300 g, 414 μmol, 1.00 eq) in THF (5.00 mL) was added lithium hydroxide monohydrate (34.7 mg, 829 μmol, 2.00 eq) and water (1.00 mL). The reaction mixture was then adjusted to pH=5 with a 1.0 M aqueous solution of HCl at 0° C. The resulting solution was concentrated under reduced pressure to afford (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-3-carboxylic acid (0.29 g, 408 μmol, 98.5% yield) as a yellow solid. LCMS [M+H]⁺=710.4 m/z.

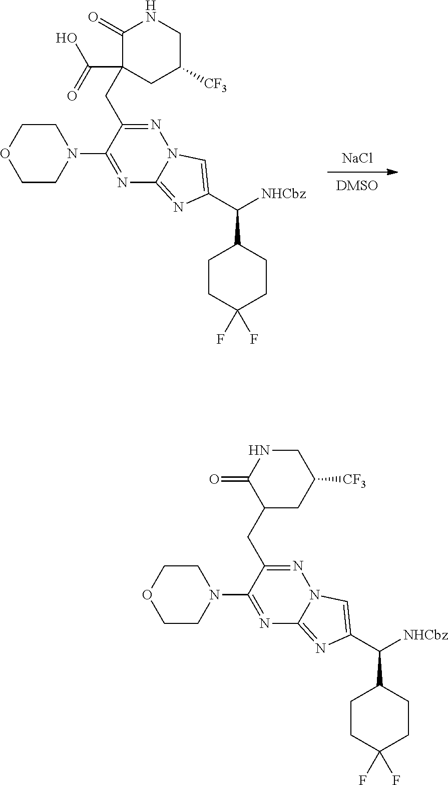

To a solution of (5R)-3-((6-((S)-(((benzyloxy)carbonyl)amino)(4,4-difluorocyclohexyl)methyl)-3-morpholinoimidazo[1,2-b][1,2,4]triazin-2-yl)methyl)-2-oxo-5-(trifluoromethyl)piperidine-3-carboxylic acid (0.290 g, 408 μmol, 1.00 eq) in dimethyl sulfoxide (1.00 mL) was added sodium chloride (95.5 mg, 1.63 mmol, 4.00 eq). The reaction mixture was stirred at 110° C. for 1 h. The reaction was cooled to RT. The reaction mixture was diluted with water and extracted with EtOAc. The combined organic layers were dried over anhydrous sodium sulfate, filtered, and concentrated under reduced pressure to afford benzyl ((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)carbamate (260 mg, 390 μmol, 95.58% yield) as a yellow solid. LCMS [M+H]⁺=666.4 m/z.

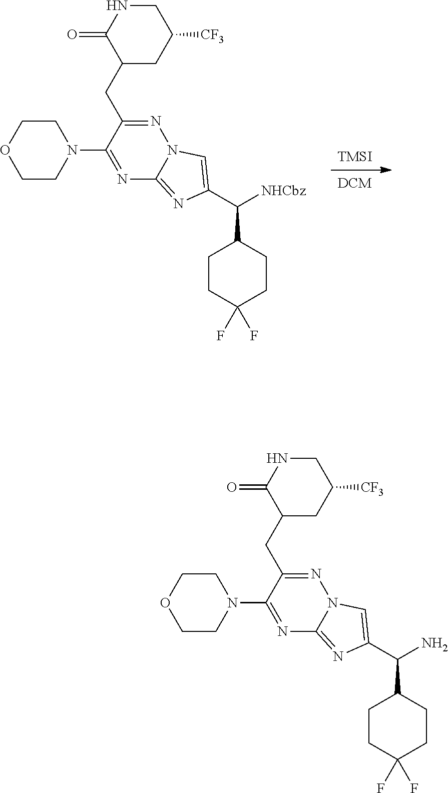

To a solution of benzyl ((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)carbamate (0.250 g, 375 μmol, 1.00 eq) in DCM (1.00 mL) was added iodotrimethylsilane (150 mg, 751 μmol, 102 μL, 2.00 eq). The reaction mixture was stirred at 0° C. for 30 min. The reaction mixture was treated with 1.0 M aqueous solution of HCl to reach pH=4 and was extracted with EtOAc. The aqueous layer was adjusted to pH=9 with a saturated aqueous solution of sodium hydrogen carbonate and was extracted with EtOAc, dried over anhydrous sodium sulfate, filtered, and concentrated under reduced pressure to afford (5R)-3-((6-((S)-amino(4,4-difluorocyclohexyl)methyl)-3-morpholinoimidazo[1,2-b][1,2,4]triazin-2-yl)methyl)-5-(trifluoromethyl)piperidin-2-one (Intermediate 4, 170 mg, 319 μmol, 85.1% yield) as a yellow solid. LCMS [M+H]⁺=532.3 m/z.

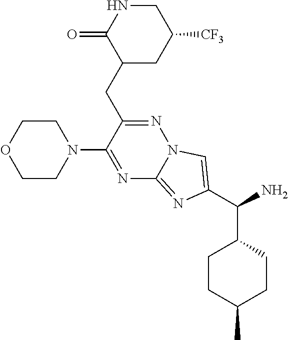 [SMILES: C[C@H]1CC[C@H]([C@H](N)c2cn3nc(CC4C[C@@H](C(F)(F)F)CNC4=O)c(N4CCOCC4)nc3n2)CC1]

### (5R)-3-((6-((S)-amino((1R,4S)-4-methylcyclohexyl)methyl)-3-morpholinoimidazo[1,2-b][1,2,4]triazin-2-yl)methyl)-5-(trifluoromethyl)piperidin-2-one (Intermediate 5)

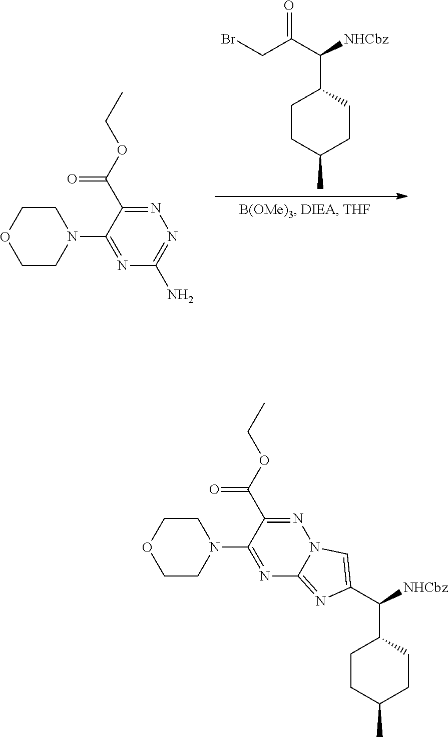

To a solution of ethyl 3-amino-5-morpholino-1,2,4-triazine-6-carboxylate (1.50 g, 5.92 mmol, 1.00 eq) in THF (10.0 mL) was added benzyl ((S)-3-bromo-1-((1R,4S)-4-methylcyclohexyl)-2-oxopropyl)carbamate (2.72 g, 7.11 mmol, 1.20 eq), trimethyl borate (3.08 g, 29.6 mmol, 3.34 mL, 5.00 eq) and N,N-diisopropylethylamine (3.83 g, 29.6 mmol, 5.16 mL, 5.00 eq). The reaction mixture was stirred at 70° C. for 1 h. The reaction was cooled to RT. The reaction mixture was filtered and concentrated under reduced pressure to give a residue, which was purified by column chromatography (SiO₂, petroleum ether/EtOAc) to afford ethyl 6-((S)-(((benzyloxy)carbonyl)amino)((1R,4S)-4-methylcyclohexyl)methyl)-3-morpholinoimidazo[1,2-b][1,2,4]triazine-2-carboxylate (2.50 g, 4.66 mmol, 78.6% yield) as a yellow gum. LCMS [M+H]⁺=537.5 m/z.

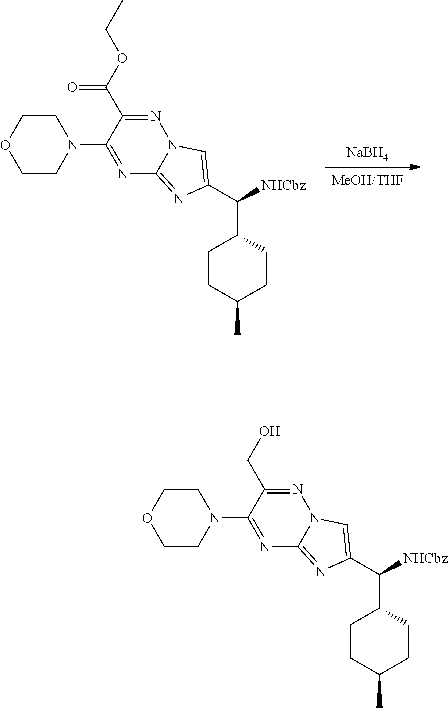 [SMILES: CCOC(=O)c1nn2cc([C@@H](NC(=O)OCc3ccccc3)[C@H]3CC[C@H](C)CC3)nc2nc1N1CCOCC1 ; "MeOH/THF" ; "NaBH4" ; C[C@H]1CC[C@H]([C@H](NC(=O)OCc2ccccc2)c2cn3nc(CO)c(N4CCOCC4)nc3n2)CC1]

To a solution of ethyl 6-((S)-(((benzyloxy)carbonyl)amino)((1R,4S)-4-methylcyclohexyl)methyl)-3-morpholinoimidazo[1,2-b][1,2,4]triazine-2-carboxylate (200 mg, 372 μmol, 1.00 eq) in THF (5.00 mL) was added sodium borohydride (50.0 mg, 1.32 mmol, 3.55 eq) at 0° C. The reaction mixture was stirred at 25° C. for 1 h. The reaction mixture was then diluted with water, adjusted to pH=7 with a saturated aqueous solution of ammonium chloride and extracted with EtOAc. The combined organic layers were dried over anhydrous sodium sulfate, filtered and concentrated under reduced pressure to give a residue, which was purified by prep-TLC (SiO₂, DCM/MeOH) to afford benzyl ((S)-(2-(hydroxymethyl)-3-morpholinoimidazo[1,2-b][1,2,4]triazin-6-yl)((1R,4S)-4-methylcyclohexyl)methyl)carbamate (40.0 mg, 80.8 μmol, 21.7% yield) as a white solid. LCMS [M+H]⁺=495.3 m/z.

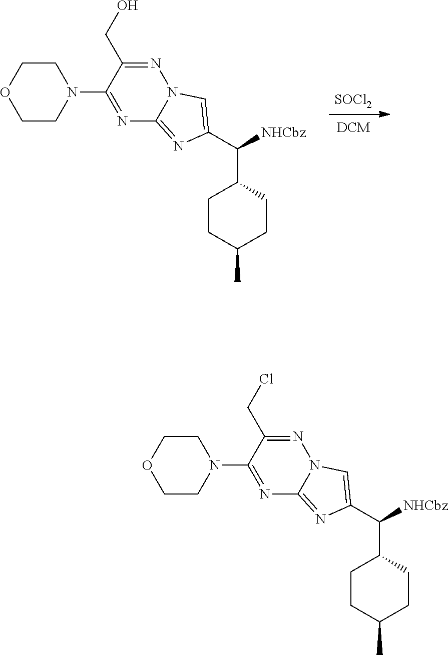

To a solution of benzyl ((S)-(2-(hydroxymethyl)-3-morpholinoimidazo[1,2-b][1,2,4]triazin-6-yl)((1R,4S)-4-methylcyclohexyl)methyl)carbamate (40.0 mg, 80.8 μmol, 1.00 eq) in DCM (5.00 mL) was added thionyl chloride (360 mg, 3.03 mmol, 220 μL, 3.00 eq) at 0° C. The reaction mixture was stirred at 25° C. for 1 h. The reaction mixture was concentrated under reduced pressure to afford benzyl ((S)-(2-(chloromethyl)-3-morpholinoimidazo[1,2-b][1,2,4]triazin-6-yl)((1R,4S)-4-methylcyclohexyl)methyl)carbamate (518 mg, 1.01 mmol, 99.8% yield) as a yellow solid. LCMS [M+H]⁺=513.3 m/z.

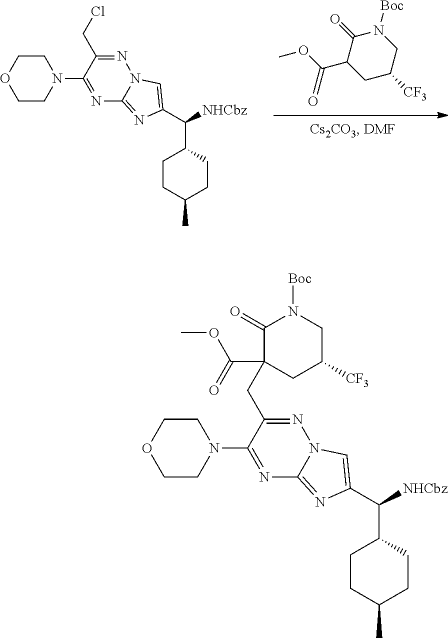

To a solution of benzyl ((S)-(2-(chloromethyl)-3-morpholinoimidazo[1,2-b][1,2,4]triazin-6-yl)((1R,4S)-4-methylcyclohexyl)methyl)carbamate (250 mg, 487 μmol, 1.00 eq) and 1-(tert-butyl) 3-methyl (5R)-2-oxo-5-(trifluoromethyl)piperidine-1,3-dicarboxylate (237 mg, 731 μmol, 1.50 eq) in N,N-dimethylformamide (4.00 mL) was added cesium carbonate (476 mg, 1.46 mmol, 3.00 eq). The reaction mixture was stirred at 25° C. for 12 h. The reaction mixture was concentrated under reduced pressure to give a residue, which was diluted with water and extracted with EtOAc. The combined organic layers were dried over anhydrous sodium sulfate, filtered and concentrated under reduced pressure to give a residue, which was purified by column chromatography (SiO₂, petroleum ether/EtOAc) to afford 1-(tert-butyl) 3-methyl (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-1,3-dicarboxylate (300 mg, 374 mol, 76.8% yield) as a yellow solid. LCMS [M+H]⁺=802.5 m/z.

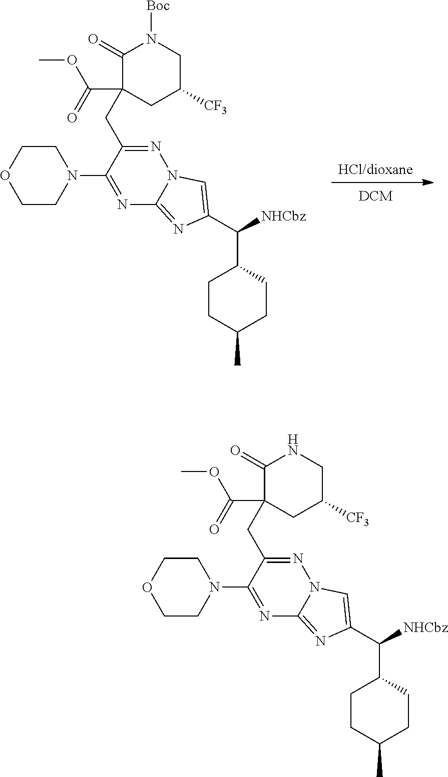

To a solution of 1-(tert-butyl) 3-methyl (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-1,3-dicarboxylate (270 mg, 337 μmol, 1.00 eq) in DCM (3.00 mL) was added a solution of HCl in dioxane (4.00 M, 3.00 mL, 35.6 eq) at 0° C. The reaction mixture was stirred at 25° C. for 1 h. The reaction mixture was concentrated under reduced pressure to afford methyl (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-3-carboxylate (200 mg, 285 μmol, 84.6% yield) as a yellow solid. LCMS [M+H]⁺=702.4 m/z.

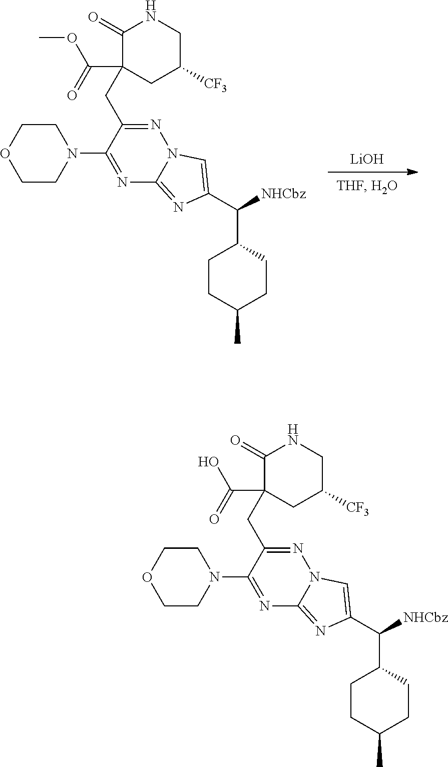

To a solution of methyl (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-3-carboxylate (200 mg, 285 μmol, 1.00 eq) in THF (4.00 mL) was added lithium hydroxide monohydrate (23.9 mg, 570 μmol, 2.00 eq) and water (0.80 mL). The reaction mixture was stirred at 25° C. for 1 h. The reaction mixture was then adjusted to pH=7 with a 1.0 M aqueous solution of HCl. The resulting solution was concentrated under reduced pressure to afford (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-3-carboxylic acid (180 mg, 262 μmol, 91.8% yield) a yellow solid.

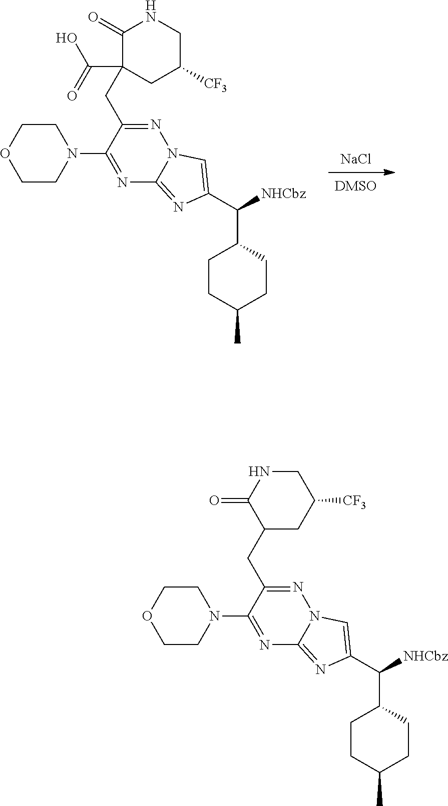

To a solution of (5R)-3-((6-((S)-(((benzyloxy)carbonyl)amino)((1R,4S)-4-methylcyclohexyl)methyl)-3-morpholinoimidazo[1,2-b][1,2,4]triazin-2-yl)methyl)-2-oxo-5-(trifluoromethyl)piperidine-3-carboxylic acid (180 mg, 261 μmol, 1.00 eq) in dimethyl sulfoxide (1.00 mL) was added sodium chloride (61.2 mg, 1.05 mmol, 4.00 eq). The reaction mixture was stirred at 110° C. for 2 h. The reaction mixture was cooled to RT. The reaction mixture was diluted with water and extracted with EtOAc. The combined organic layers were dried over anhydrous sodium sulfate, filtered and concentrated under reduced pressure to give a residue, which was purified by prep-TLC (SiO₂, DCM/MeOH) to afford benzyl ((1S)-((1R,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)carbamate (145 mg, 225 μmol, 86.0% yield) as a yellow solid. LCMS [M+H]⁺=644.4 m/z.

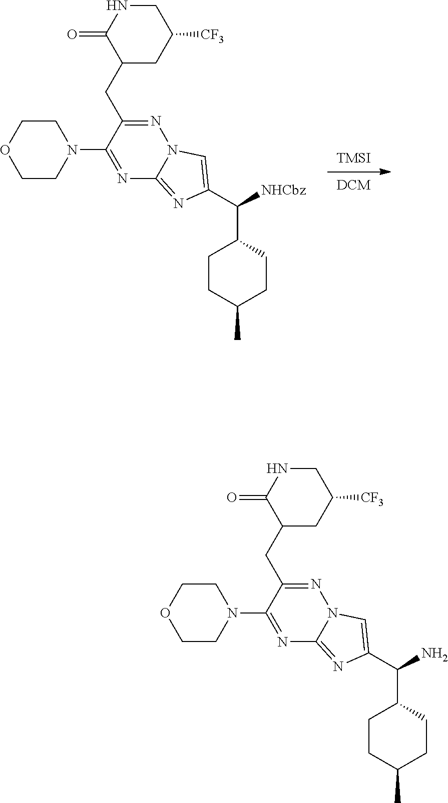

To a solution of benzyl ((1S)-((1R,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)carbamate (130 mg, 202 μmol, 1.00 eq) in DCM (1.00 mL) was added iodotrimethylsilane (121 mg, 606 μmol, 82.4 μL, 3.00 eq) at 0° C. The reaction mixture was stirred at 25° C. for 2 h. The reaction mixture was quenched by the addition of a 1.0 M aqueous solution of HCl (pH=4) and extracted with DCM. The aqueous layer was adjusted to pH=8 with a saturated aqueous solution of sodium hydrogen carbonate and was extracted with DCM, dried over anhydrous sodium sulfate, filtered and concentrated under reduced pressure to afford (5R)-3-((6-((S)-amino((1R,4S)-4-methylcyclohexyl)methyl)-3-morpholinoimidazo[1,2-b][1,2,4]triazin-2-yl)methyl)-5-(trifluoromethyl)piperidin-2-one (Intermediate 5, 100 mg, 196 μmol, 97.1% yield) as a yellow solid. LCMS [M+H]⁺=510.4 m/z.

### General Procedure 2

To a solution of Intermediate 4 or 5 (1.00 eq) and carboxylic acid in pyridine (3.00 mL) was added EDCI (3.00 eq). The reaction mixture was stirred at 25° C. for 1-2 h. The reaction mixture was diluted with water and extracted with EtOAc. The combined organic layers were dried over anhydrous sodium sulfate, filtered, and concentrated under reduced pressure to give a residue, which was purified by prep-TLC followed by prep-SFC to afford the final compound.

### Example 25: Preparation of N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 17, Compound 18, and Compound 19)

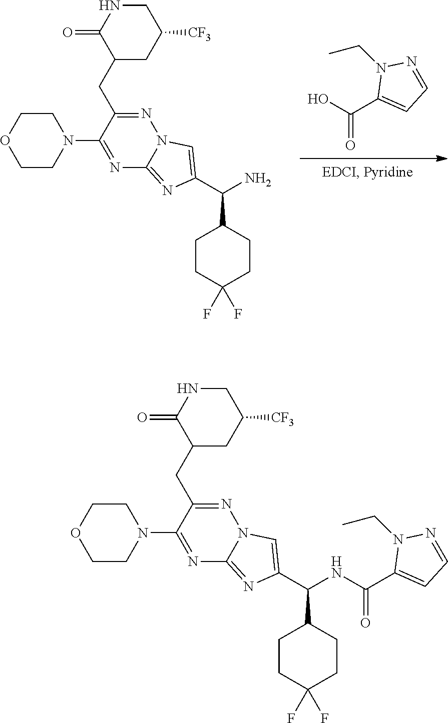

Compound 17 was synthesized following General Procedure 2, employing Intermediate 4 and 1-ethyl-1H-pyrazole-5-carboxylic acid (23.7 mg, 169 μmol, 1.50 eq), and purified by prep-TLC (SiO₂, DCM/MeOH) followed by chiral SFC purification (column: Daicel Chiralcel OX (250 mm×30 mm, 10 um); mobile phase: [CO₂-EtOH (0.1% NH₃·H₂O)]; B %: 40%, isocratic elution mode) to afford as the first eluting, single stereoisomer N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (29.41 mg, 43.97 μmol, 38.95% yield, 95.8% purity) as a yellow solid. LCMS [M+H]⁺=654.4 m/z.

Compound 18 was isolated as the second eluting, single stereoisomer by chiral SFC purification (18.84 mg, 29.86 μmol, 26.45% yield, 97.6% purity) as a yellow solid. LCMS [M+H]⁺=654.4 m/z. Compound 19 was isolated as mixture of isomers.

### Example 26: Preparation of 4-cyclopropyl-N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 20 and Compound 21)

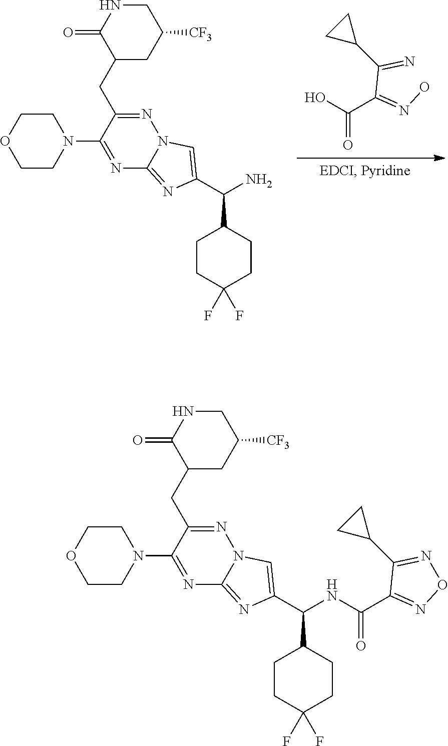

Compound 20 was synthesized following General Procedure 2, employing Intermediate 4 and 4-cyclopropyl-1,2,5-oxadiazole-3-carboxylic acid (39.1 mg, 253 μmol, 1.50 eq), and purified by prep-TLC (SiO₂, DCM/MeOH) followed by chiral SFC purification (column: Daicel Chiralcel OX (250 mm×30 mm, 10 um); mobile phase: [CO₂-EtOH (0.1% NH₃·H₂O)]; B %: 40%, isocratic elution mode) to afford as the first eluting, single stereoisomer 4-cyclopropyl-N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1,2,5-oxadiazole-3-carboxamide (39.66 mg, 50.4 μmol, 29.8% yield, 96.3% purity) as a yellow solid. LCMS [M+H]⁺=668.4 m/z.

Compound 21 was isolated as the second eluting, single stereoisomer by chiral SFC purification (23.82 mg, 51.1 μmol, 30.1% yield, 97.5% purity) as a yellow solid. LCMS [M+H]⁺=668.4 m/z.

### Example 27: Preparation of 1-ethyl-N-((1S)-(1R,4S)-4-methylcyclohexyl)(3-morpholino-2-(((5R)-2-oxo-5-trifluoromethyl)piperidin-3-yl)methyl)imidazo[1,2-b][1,2,4]triazin-6-yl)methyl)-1H-pyrazole-5-carboxamide (Compound 22 and Compound 23)

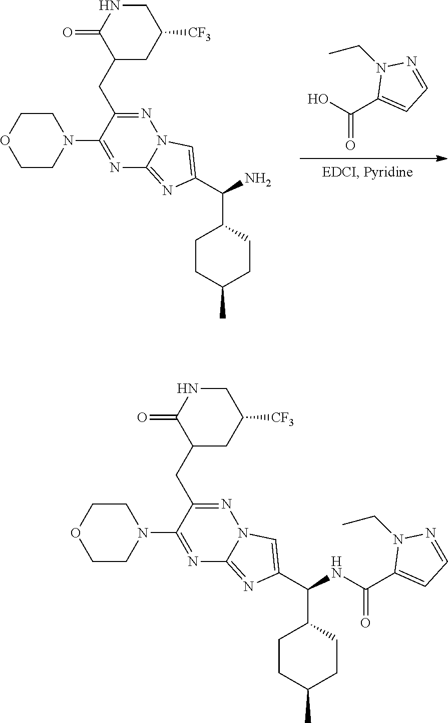

Compound 22 was synthesized following General Procedure 2, employing Intermediate 5 and 1-ethyl-1H-pyrazole-5-carboxylic acid (41.2 mg, 294 μmol, 1.50 eq), and purified by prep-TLC (SiO₂, DCM/MeOH) to afford 1-ethyl-N-((1S)-((1R,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)-1H-pyrazole-5-carboxamide (50.0 mg, 79.1 μmol, 40.3% yield) as a yellow solid. LCMS [M+H]⁺=632.3 m/z.

Compound 22 was isolated as the first eluting, single stereoisomer by chiral SFC purification (column: Daicel Chiralcel OD (250 mm×30 mm, 10 um); mobile phase: [CO₂-0.1% NH₃·H₂O in EtOH]; B %: 60%, isocratic elution mode); (26.5 mg, 42.0 μmol, 53.1% yield, 95.8% purity) and was obtained as a yellow solid. LCMS [M+H]⁺=632.4 m/z.

Compound 23 was isolated as the second eluting, single stereoisomer by chiral SFC purification (column: Daicel Chiralcel OD (250 mm×30 mm, 10 um); mobile phase: [CO₂-0.1% NH₃·H₂O in EtOH]; B %: 60%, isocratic elution mode); (21.1 mg, 33.4 μmol, 42.3% yield, 96.0% purity) and was obtained as a yellow solid. LCMS [M+H]⁺=632.4 m/z.

### Example 28: General Scheme 1 and 2

General Scheme 1 and 2

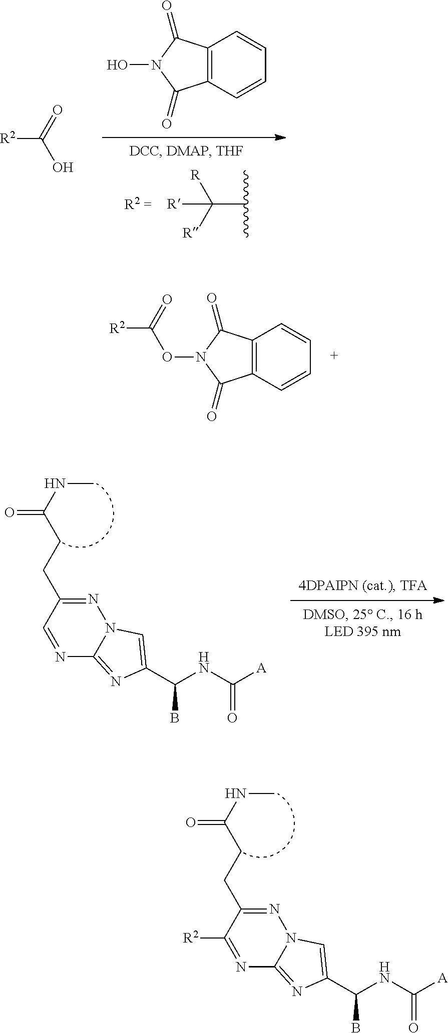

As described in General Scheme 1, R²

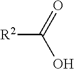

of OH is selected from R² as defined in Formula (A), (I′), (I), (II), (II-a), or (II-b). In some embodiments, R² of

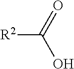

is selected from C₁₋₁₀ alkyl and C₂₋₁₀ alkenyl, each of which is optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, —N(R^{15A}), and —CN, wherein R¹⁵ and R^{15A} are as defined in Formula (I′) or Formula (I).

In some embodiments, R² of

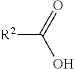

is selected from C₂₋₁₀ alkenyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² of

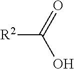

is selected from C₂₋₁₀ alkenyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle. In some embodiments, R² of

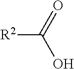

is selected from C₂₋₆ alkenyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN, C₃₋₁₀ carbocycle and 3- to 10-membered heterocycle. In some embodiments, R² of

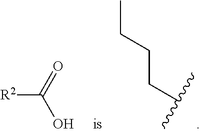

In some embodiments, R² of

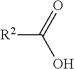

is selected from C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle, the C₃₋₁₀ carbocycle and 3- to 10-membered heterocycle are each optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R¹⁵, —NO₂, ═O, ═S, ═N(R^{15A}), and —CN. In some embodiments, R² of

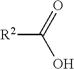

is selected from C₁₋₁₀ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, ═O, ═S, ═N(R¹⁵), —CN, C₃₋₁₀ carbocycle and 3- to 10-membered heterocycle. In some embodiments, R² of

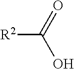

is selected from C₁₋₆ alkyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —NO₂, —CN, C₃₋₁₀ carbocycle and 3- to 10-membered heterocycle. In some embodiments, R² of

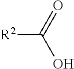

is selected from C₁₋₆ alkyl optionally substituted with one or more substituents independently selected from halogen, —OR¹⁵, —N(R¹⁵)₂, —C(O)R¹⁵, —NO₂, and —CN. In some embodiments, R² of

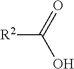

is selected from,

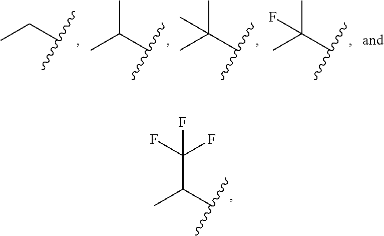

and or stereoisomers thereof.

In some embodiments, R² of

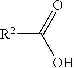

is selected from C₁₋₁₀ alkyl optionally substituted with one or more substituents independently selected C₃₋₁₀ carbocycle and 3- to 10-membered heterocycle, each of which optionally substituted with one or more substituents independently selected from halogen, —OR^{15A}, —SR^{15A}, —N(R^{15A})₂, —C(O)R^{15A}, —C(O)N(R^{15A})₂, —N(R^{15A})C(O)R^{15A}, —N(R^{15A})S(O)₂R^{15A}, —C(O)OR^{15A}, —OC(O)R^{15A}, —S(O)R^{15A}, —S(O)₂R^{15A}, —NO₂, ═O, ═S, ═N(R^{15A}), and —CN. In some embodiments, R² of

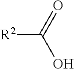

is selected from C₁₋₁₀ alkyl optionally substituted with one or more substituents independently selected C₃₋₁₀ carbocycle and 3- to 10-membered heterocycle. In some embodiments, R² of

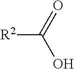

is selected from

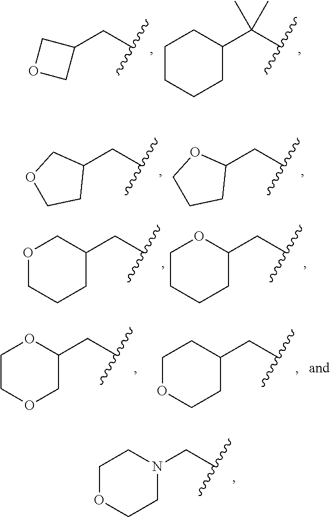

or stereoisomers thereof.

In some embodiments, R² of

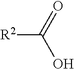

is selected from

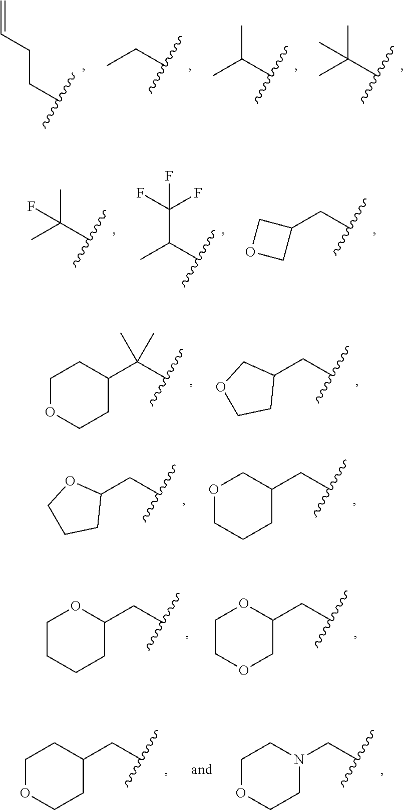

or stereoisomers thereof.

As described in General Scheme 1, A is as defined in Formula (A), (I′), (I), (II), (II-a), or (II-b). In some embodiments, A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted as defined as in Formula (I′) or Formula (I), and wherein z is as defined as in Formula (I′) or Formula (I). In some embodiments, A is selected from:

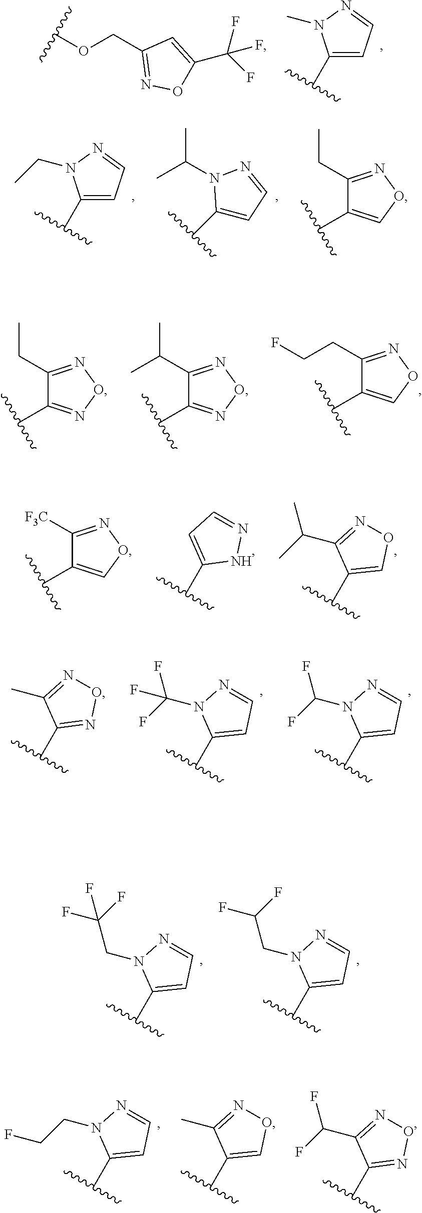

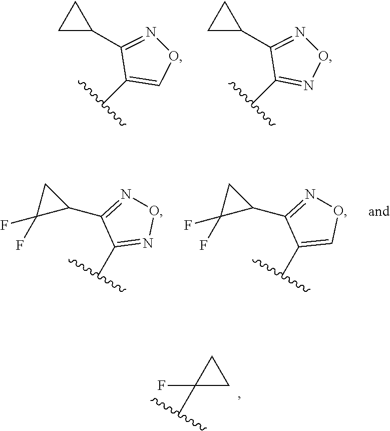

stereoisomers thereof.

As described in General Scheme 1, B is as defined in Formula (A), (I′), (I), (II), (II-a), or (II-b). In some embodiments, B is selected from —CH(R⁶)₂ and optionally substituted C₃₋₁₀ carbocycle, wherein each R⁶ and the optionally substituents of the C₃₋₁₀ carbocycle are as defined in Formula (I′) or Formula (I). In some embodiments, B is selected from

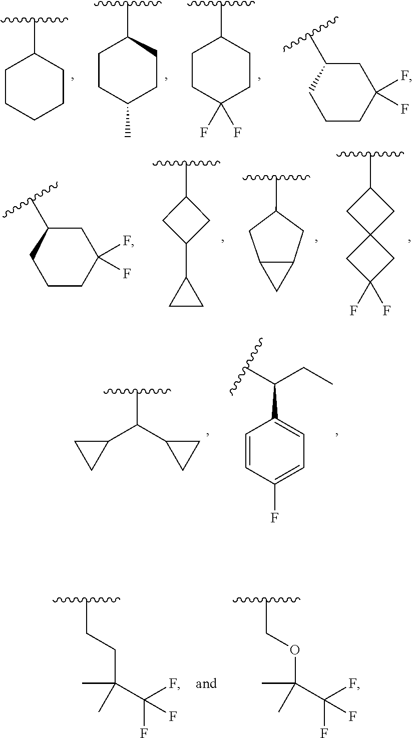

or stereoisomers thereof.

As described in General Scheme 1,

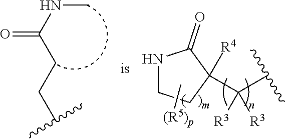

as defined in Formula (I′) or Formula (I), wherein R³, R⁴, R⁵, n, m, and p are each as defined as in Formula (I′) or Formula (I).

### General Scheme 2

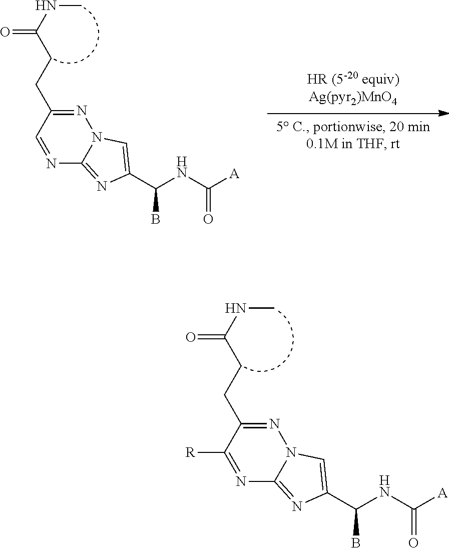

As described in General Scheme 2, R of HR, is R² as defined in Formula (I′) or Formula (I). In some embodiments, R of HR, is —N(R¹⁵)₂ of R² as defined in Formula (I′) or Formula (I), and wherein each R¹⁵ is as defined in Formula (I′) or Formula (I). In some embodiments,

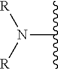

is —N(R¹⁵)₂ of R² as defined in Formula (I′) or Formula (I), and wherein each R¹⁵ is as defined in Formula (I′) or Formula (I).

In some embodiments, R of HR, is —N(R¹⁵)₂ of R² as defined in Formula (I′) or Formula (I), and wherein each R¹⁵ is selected from C₁₋₆ alkyl optionally substituted with one or more substituents independently selected from halogen, —O—C₁₋₆ alkyl, —O—C₁₋₆ haloalkyl, —NH₂, —NO₂, ═O, —CN; C₃₋₆ carbocycle and 4- to 6-membered heterocycle, the C₃₋₆ carbocycle and 4- to 6-membered heterocycle are each optionally substituted with one or more substituents independently selected from: halogen, —OH, —O—C₁₋₆ alkyl, —O—C₁₋₆ haloalkyl, —NH₂, —NO₂, ═O, and —CN. In some embodiments, R of HR is selected from:

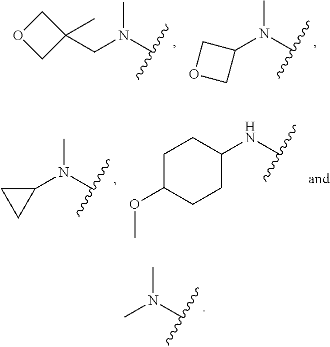

In some embodiments,

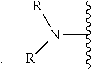

is —N(R¹⁵)₂ of R² as defined in Formula (I′) or Formula (I), and wherein each R¹⁵ is selected from C₁₋₆ alkyl optionally substituted with one or more substituents independently selected from halogen, —O—C₁₋₆ alkyl, —O—C₁₋₆ haloalkyl, —NH₂, —NO₂, ═O, —CN; C₃₋₆ carbocycle and 4- to 6-membered heterocycle, the C₃₋₆ carbocycle and 4- to 6-membered heterocycle are each optionally substituted with one or more substituents independently selected from: halogen, —OH, —O—C₁₋₆ alkyl, —O—C₁₋₆ haloalkyl, —NH₂, —NO₂, ═O, and —CN. In some embodiments,

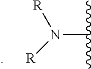

is selected from:

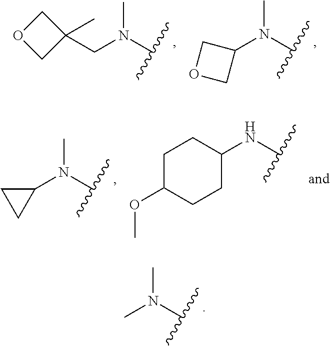

As described in General Scheme 2, A is as defined in Formula (A), (I′), (I), (II), (II-a), or (II-b). In some embodiments, A is selected from —(CR′R″)_{z}(3- to 10-membered heterocycle), —(CR′R″)_{z}(C₃₋₁₀ carbocycle), —O—(CR′R″)_{z}(3- to 10-membered heterocycle), and —O—(CR′R″)_{z}(C₃₋₁₀ carbocycle), the 3 to 10-membered heterocycle and C₃₋₁₀ carbocycle are each optionally substituted as defined as in Formula (I′) or Formula (I), and wherein z is as defined as in Formula (I′) or Formula (I). In some embodiments, A is selected from:

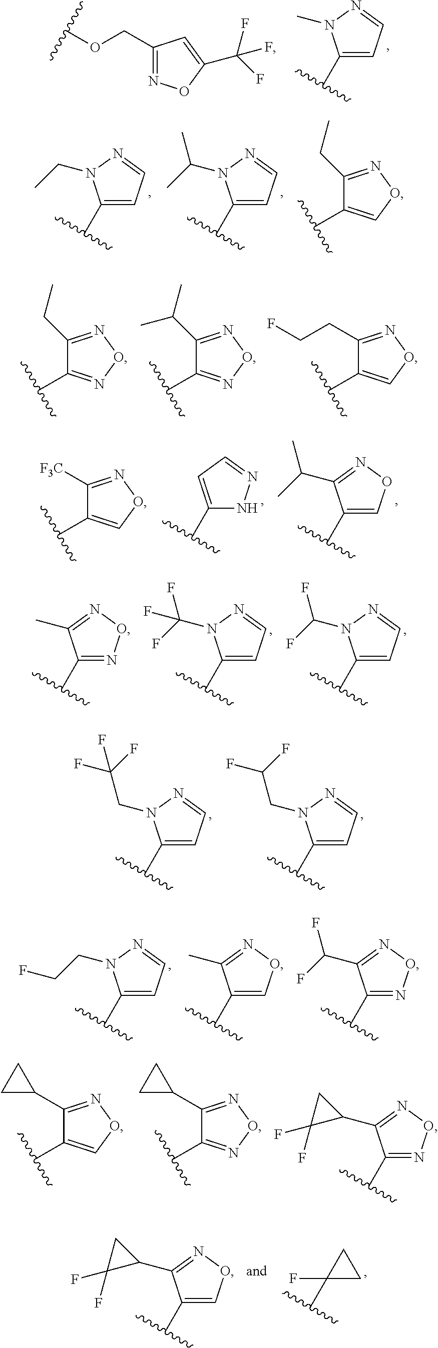

or stereoisomers thereof.

As described in General Scheme 2, B is as defined in Formula (A), (I′), (I), (II), (II-a), or (II-b). In some embodiments, B is selected from —CH(R⁶)₂ and optionally substituted C₃₋₁₀ carbocycle, wherein each R⁶ and the optionally substituents of the C₃₋₁₀ carbocycle are as defined in Formula (I′) or Formula (I). In some embodiments, B is selected from:

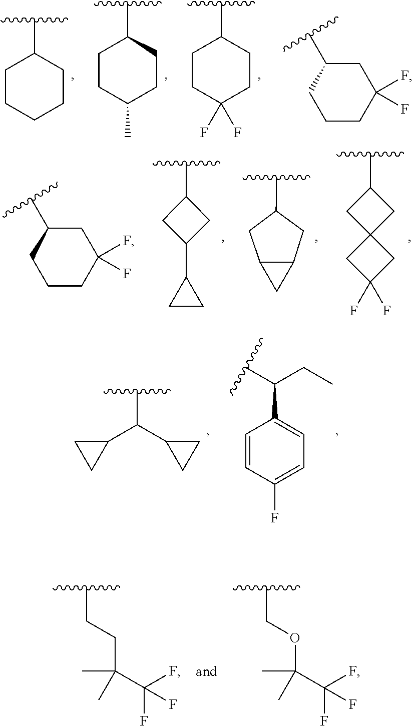

or stereoisomers thereof.

As described in General Scheme 2,

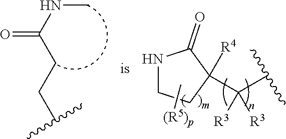

as defined in Formula (I′) or Formula (I), wherein R³, R⁴, R, n, m, and p are each as defined as in Formula (I′) or Formula (I).

### Example 29: General Procedure 3

### General Procedure 3

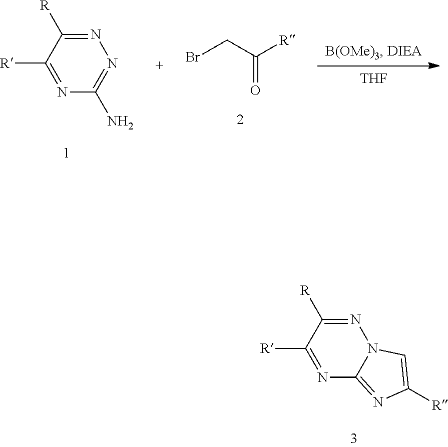

To a solution of aminotriazine 1 (1.00 eq) and bromoketone 2 (1.00-1.20 eq) in tetrahydrofuran was added N,N-diisopropylethylamine (5.00-10.0 eq) and trimethyl borate (5.00-10.0 eq). The reaction mixture was stirred at 70° C. for 3-18 h. The reaction mixture was then diluted with water and extracted with ethyl acetate. The combined organic layers were washed with water then brine, dried over anhydrous sodium sulfate, filtered, and concentrated under reduced pressure to give a residue, which was then purified by prep-TLC, column chromatography, prep-HPLC, and/or prep-SFC to afford imidazo[1,2-b][1,2,4]triazine 3.

### Example 30: General Procedure 4

### General Procedure 4

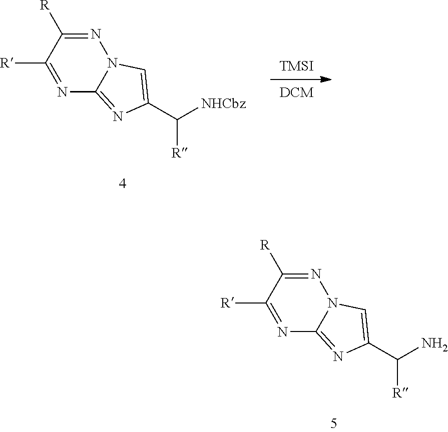

To a solution of Cbz-protected primary amine 4 (1.00 eq) in dichloromethane was added iodotrimethylsilane (3.00-5.00 eq) at 0° C. The mixture was stirred at 20° C. for 2 h. The reaction mixture was diluted with 1.0 M aqueous solution of hydrochloric acid and washed with ethyl acetate. The aqueous phase was adjusted to pH=8-9 with a saturated aqueous solution of sodium hydrogen carbonate and extracted with ethyl acetate. The combined organic layers were dried over anhydrous sodium sulfate, filtered, and concentrated under reduced pressure to afford free primary amine 5.

### Example 31: General Procedure 5

### General Procedure 5

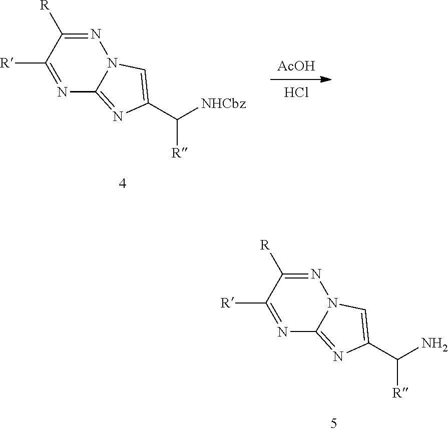

To a solution of Cbz-protected primary amine 4 (1.00 eq) in acetic acid was added concentrated HCl (aqueous 37.0%). The reaction mixture was stirred at 60° C. for 0.5-1 h. The reaction mixture was cooled to room temperature. The pH was adjusted to 8-9 with a saturated aqueous solution of sodium hydrogen carbonate and extracted with ethyl acetate. The combined organic layers were dried over anhydrous sodium sulfate, filtered, and concentrated under reduced pressure to afford free primary amine 5.

### Example 32: General Procedure 6

### General Procedure 6

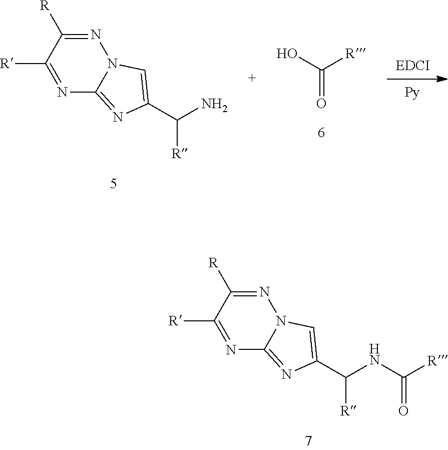

A solution of primary amine 5 (1.00 eq), carboxylic acid 6 (1.20-3.00 eq), and EDCI (2.00-3.00 eq) in pyridine were stirred at room temperature for 1-18 h. The reaction mixture was then diluted with water and extracted with ethyl acetate. The combined organic layers were optionally washed with water then brine, dried over anhydrous sodium sulfate, filtered, and concentrated under reduced pressure to give a residue, and the residue was purified by prep-TLC, column chromatography, prep-HPLC, and/or prep-SFC to afford amide 7.

### Example 33: General Procedure 7

### General Procedure 7

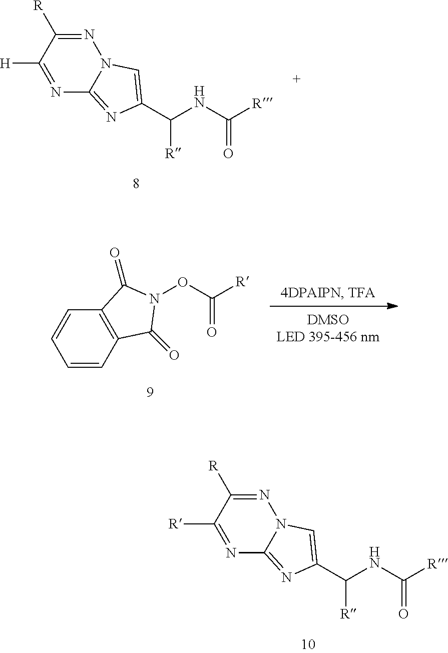

To a solution of imidazo[1,2-b][1,2,4]triazine 8 (1.00 eq), activated ester 9 (1.50-3.00 eq), and 1,3-dicyano-2,4,5,6-tetrakis(diphenylamino)-benzene (4DPAIPN, 0.02 eq) in DMSO was added TFA (1.50-3.00 eq). The reaction mixture was degassed and purged with N₂. The reaction mixture was stirred at room temperature for 16 h while irradiating with a LED (395-456 nm). The reaction mixture was then diluted with water and extracted with ethyl acetate. The combined organic layers were optionally washed with water then brine, dried over anhydrous sodium sulfate, filtered, and concentrated under reduced pressure to give a residue, and the residue was purified by prep-TLC, column chromatography, prep-HPLC, and/or prep-SFC to afford amide 10.

### Example 34: General Procedure 8

### General Procedure 8

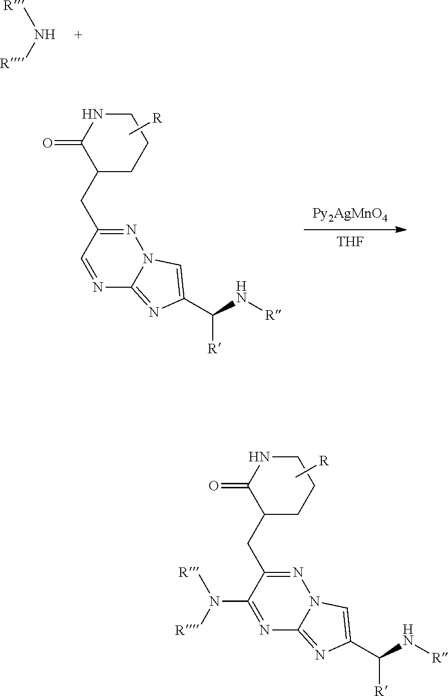

To a solution of imidazo[1,2-b][1,2,4]triazine derivative (1.00 eq) and amine (10.0-20.0 eq) in THF (0.05-0.2 M) at 0-5° C. was added bis(pyridine)silver(I) permanganate (1.50 eq) in small portions over the course of 15 min. Additional bis(pyridine)silver(I) permanganate was added at 5° C. until the reaction was complete. The reaction mixture was then diluted with dichloromethane and filtered through packed MgSO₄ atop Celite with dichloromethane/ethyl acetate washes. Purification by column chromatography, prep-TLC, and/or prep-HPLC, optionally followed by basic workup (for freebase), afforded the corresponding 7-(amino)imidazo[1,2-b][1,2,4]triazine derivative.

### Example 35: General Scheme A

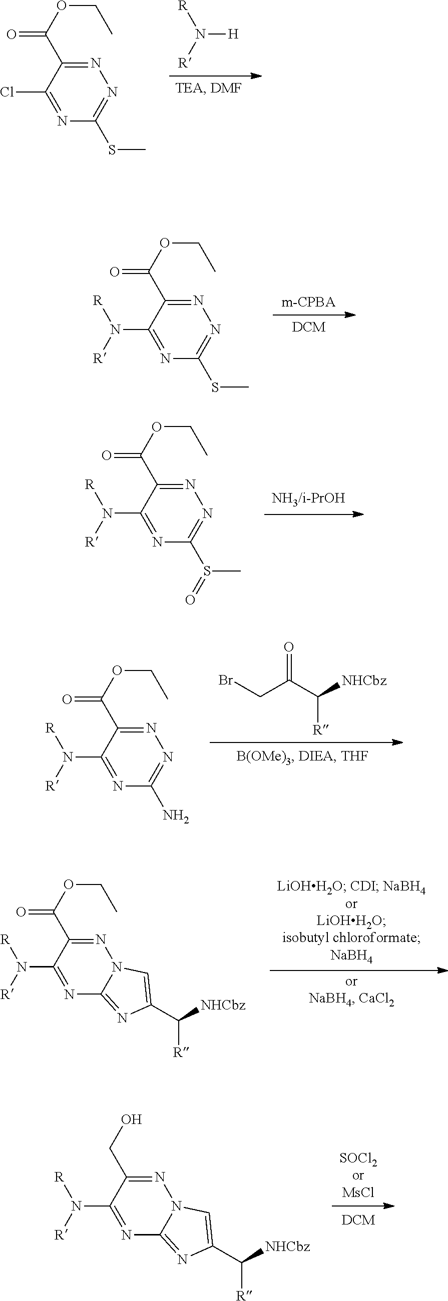

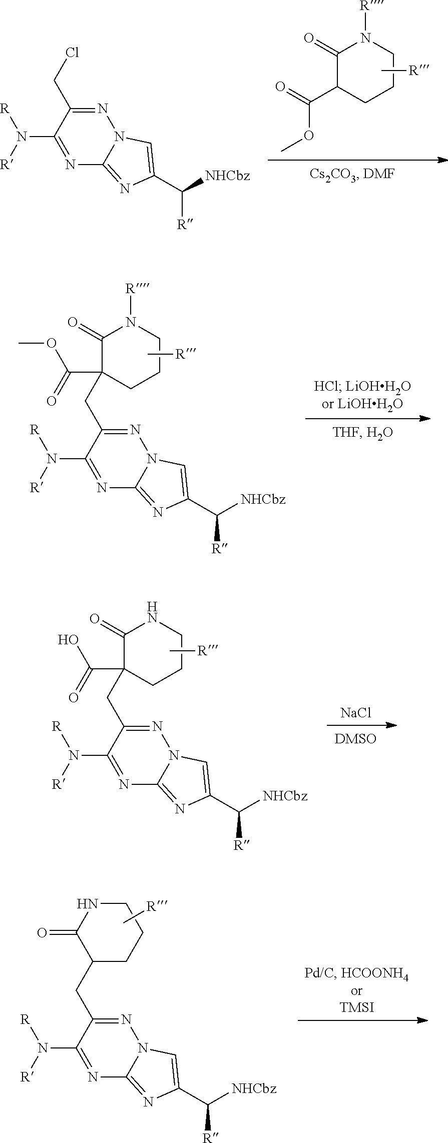

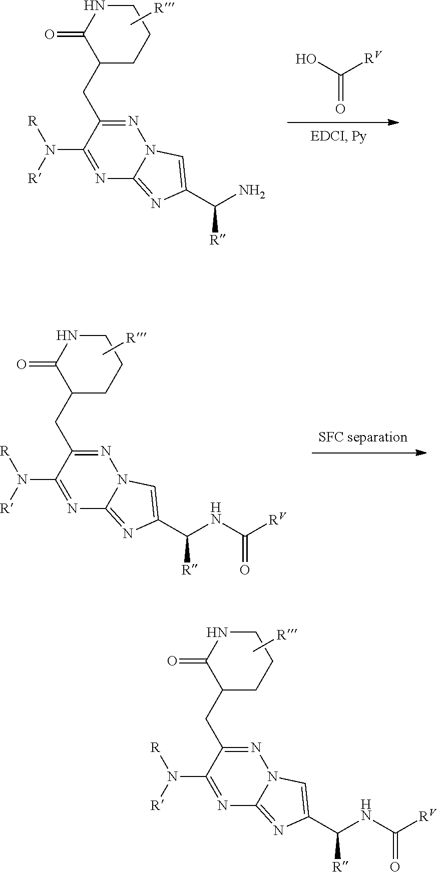

Step 1: To a solution of ethyl 5-chloro-3-(methylthio)-1,2,4-triazine-6-carboxylate (1.00 eq) and amine (1.00 eq) in dichloromethane (0.5 M) was added TEA (2.50 eq). The reaction mixture was stirred at 15° C. for 1 h then diluted with H₂O and extracted with ethyl acetate (3×). The combined organic layers were dried over Na₂SO₄, filtered, and concentrated under reduced pressure to give crude S_{N}Ar product.

Step 2: To crude S_{N}Ar product (1.00 eq) in dichloromethane (0.4 M) was added m-CPBA (1.30 eq). The reaction mixture was stirred at 0° C. for 1 h. This crude oxidation mixture was used in the next step.

Step 3: Crude oxidation mixture (1.00 eq) and NH₃/i-PrOH (7 M, 3.00 eq) were stirred at 15° C. for 1 h. The reaction mixture was filtered and concentrated under reduced pressure to give a residue, which was purified by column chromatography to afford diamino-triazine.

Step 4: Diamino-triazine was subjected to imidazole cyclization with Cbz-protected (amino)bromoketone (0.70-1.00 eq) using General Procedure 3. Upon purification by column chromatography, ethyl imidazo[1,2-b]triazine-carboxylate derivative was obtained as the product.

Step 5: The synthesis of benzylic alcohol from ethyl imidazo[1,2-b]triazine-carboxylate derivative was carried out by one of three procedures: A) To a solution of ethyl imidazo[1,2-b]triazine-carboxylate derivative (1.00 eq) in THF (0.25 M) was added LiOH·H₂O (2.00-3.00 eq) and H₂O (1/10 THF vol). The mixture was stirred at 0-room temperature for 1-2 h. The reaction mixture was then diluted with H₂O and pH was adjust to 4 with HCl (1 M). Then the mixture was extracted with ethyl acetate (3×). The combined organic layers were dried over with Na₂SO₄, filtered, and concentrated under reduced pressure to give lactam-carboxylic acid. A 0.2 M solution of lactam-carboxylic acid in THF with isobutyl chloroformate (3.00-4.00 eq) and TEA (3.00 eq) was stirred at room temperature for 20 min then filtered and concentrated, the residue taken up into solution with THF/H₂O (5:1, 0.13 M), stirred with NaBH₄ (2.00-5.00 eq, 0° C.) at room temperature for 30 min, and the reaction mixture quenched with sat. aq. NH₄Cl and water, extracted with ethyl acetate (3×), and the combined organic layers wash with brine (3×), dried with Na₂SO₄, filtered, concentrated, and purified by column chromatography (SiO₂) to give benzylic alcohol. Or B) A solution of lactam-carboxylic acid (made in same way as Step 5A) in THF (0.15 M) was stirred with CDI (3.00 eq) at 25-45° C. for 2-12 h, followed by addition of NaBH₄ (2.00-2.50 eq) and H₂O (1/27 THF vol) at 0° C., stirring for 0.5 h, quenching at 0° C. with sat. aq. NH₄C₁ and H₂O, extraction with ethyl acetate (4×), drying combined organic layers over Na₂SO₄, filtering concentrating, and purifying by column chromatography (SiO₂) to give benzylic alcohol. Or C) A solution of ethyl imidazo[1,2-b][1,2,4]triazine-carboxylate derivative (1.00 eq) in EtOH or MeOH (0.19-0.26 M) was added NaBH₄ (2.00-3.50 eq) and CaCl₂ (1.00 eq) at 0° C. The reaction mixture was stirred at 15-room temperature for 1 h. The reaction mixture was quenched with sat. aq. NH₄Cl 200 mL, then optionally concentrated under reduced pressure to remove MeOH and diluted with water. The aqueous phase was extracted with ethyl acetate (3×) and the combined organic layers were dried over anhydrous Na₂SO₄, filtered, and concentrated under reduced pressure to give a residue. The residue was purified by column chromatography to afford benzylic alcohol product.

Step 6: The synthesis of benzylic chloride from benzylic alcohol was carried out via one of the two following procedures: A) A solution of benzylic alcohol (1.00 eq) in dichloromethane (0.1-0.2 M) was stirred with TEA (4.00 eq) and MsCl (4.00-9.00 eq) at 0° C. for 1 h, then concentrated directly to give benzylic chloride. Or B) A solution of benzylic alcohol (1.00 eq) in dichloromethane (0.15 M) was stirred with thionyl chloride (3.00-5.00 eq, 0° C.) at room temperature for 1 h then concentrated directly to give benzylic chloride.

Step 7: To a solution of 1,3-dicarbonyl-lactam (0.33-2.00 eq) in THF (0.6 M) was added Cs₂CO₃ (1.00-2.00 eq) at room temperature. The mixture was stirred at room temperature for 30 min, then was added benzylic chloride (1.00-3.00 eq) and was stirred at 15-70° C. for 2-5 h. The mixture was diluted with H₂O and extracted with ethyl acetate (3×). The combined organic layers were dried over by Na₂SO₄, filtered, and concentrated under reduced pressure to give a residue, which was purified by prep-TLC to give lactam methy product.

Step 8: If alkylated lactam product from previous step was Boc protected, deprotection was carried out via the following procedure prior to saponification: A solution of Boc-protected lactam (1.00 eq) in dichloromethane (0.1 M) was added 4 M HCl/dioxane (20-35 eq) at 0° C., then the reaction mixture was stirred at room temperature for 1 h. The reaction mixture was concentrated under the vacuum to give crude unprotected lactam methyl ester. Saponification procedure: To a solution of unprotected lactam methyl ester (1.00 eq) in THF/H₂O (5-6:1, 0.09-0.15 M) was added LiOH·H₂O (2.00-5.00 eq) at room temperature. The mixture was stirred at room temperature for 2 h then optionally acidified to pH=7 with aq. HCl and concentrated under reduced pressure to dryness to give crude lactam-carboxylic acid (Li salt or freebase).

Step 9: To a solution of crude lactam-carboxylic acid (Li salt or freebase, 1.00 eq) in DMSO (0.15 M) was added NaCl (2.00-3.00 eq) and stirred at 25-80° C. for 0.5 h. The reaction mixture was then diluted with H₂O and extracted with ethyl acetate (2-3×). The combined organic layers were dried over Na₂SO₄, filtered, and concentrated under reduced pressure and optionally purified by prep-TLC to give decarboxylated CBz-amine product.

Step 10: Cbz-deprotection of decarboxylated CBz-amine was carried out by one of two procedures. A) To a solution of decarboxylated CBz-amine (1.00 eq) in MeOH (0.06-0.1 M) was added HCOONH₄ (3.00-20.0 eq) and Pd/C (10.0% purity, 0.15-7.80 eq), and the mixture was stirred at 15-room temperature for 2 h, optionally under N₂, then filtered, and concentrated under reduced pressure to give benzylic amine. Or B) A solution of decarboxylated CBz-amine (1.00 eq) in dichloromethane (0.075 M) with TMSI (3.00 eq, 0° C.) was stirred at room temperature for 1 h, diluted with water, acidified to pH=3 (1 M HCl), washed with ethyl acetate (2×), basified to pH=9 (sat. aq. NaHCO₃), and extracted with ethyl acetate (3×); the combined organic layers were then dried over anhydrous Na₂SO₄, filtered, and concentrated under reduced pressure to afford benzylic amine.

Step 11: Benzylic amine from the previous step was coupled with carboxylic acid using General Procedure 6 to afford the desired amide product as a crude mixture of diastereomers that were further purified by prep-TLC, prep-HPLC, and/or column chromatography and subsequently separated by chiral SFC to give individual stereoisomers.

### Example 36: Preparation of N-((1S)-(4,4-difluorocyclohexyl)(3-(dimethylamino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 48 and 49)

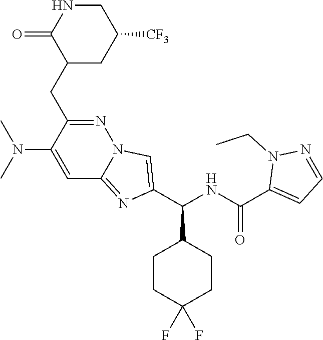 [SMILES: CCn1nccc1C(=O)N[C@H](c1cn2nc(CC3C[C@@H](C(F)(F)F)CNC3=O)c(N(C)C)cc2n1)C1CCC(F)(F)CC1]

The synthesis of individual stereoisomers of N-((1S)-(4,4-difluorocyclohexyl)(3-(dimethylamino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using dimethylamine hydrochloride (1.70 eq) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, 1-(tert-butyl) 3-methyl (5R)-2-oxo-5-(trifluoromethyl)piperidine-1,3-dicarboxylate in step 7, HCl/dioxane prior to saponification in step 8, TMSI for Step 10B, and 1-ethyl-1H-pyrazole-5-carboxylic acid in step 11. The mixture of stereoisomers was purified by prep-TLC (10:1 dichloromethane/MeOH) and individual stereoisomers were subsequently separated by prep-SFC (column: REGIS(S,S) WHELK-O1 (250 mm*25 mm, 10 μm); mobile phase: [CO₂-MeOH (0.1% NH₃H₂O)]; B %: 55%, isocratic elution mode). The first eluting single stereoisomer, Compound 48 (29.28 mg, 46.9 μmol), was obtained as a yellow solid. LCMS [M+H]⁺=612.3 nm/z.

The second eluting single stereoisomer, Compound 49 (30.01 mg, 48.4 μmol) was obtained as a yellow solid. LCMS [M+H]⁺=612.3 m/z.

### Example 37: Preparation of N-((1S)-(4,4-difluorocyclohexyl)(3-morpholino-2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 50 and 54)

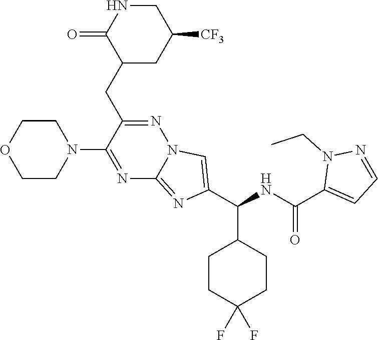 [SMILES: CCn1nccc1C(=O)N[C@H](c1cn2nc(CC3C[C@H](C(F)(F)F)CNC3=O)c(N3CCOCC3)nc2n1)C1CCC(F)(F)CC1]

The synthesis of individual stereoisomers of N-((1 S)-(4,4-difluorocyclohexyl)(3-morpholino-2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using morpholine (1.10 eq, freebase) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, 1-(tert-butyl) 3-methyl (5S)-2-oxo-5-(trifluoromethyl)piperidine-1,3-dicarboxylate in step 7, HCl/dioxane prior to saponification in step 8, TMSI for Step 10B, and 1-ethyl-1H-pyrazole-5-carboxylic acid in step 11 The mixture of stereoisomers was purified by prep-TLC (10:1 dichloromethane/MeOH) and individual stereoisomers were subsequently separated by prep-SFC (column: DAICEL CHIRALPAK AS (250 mm*30 mm, 10 μm); mobile phase: [CO₂-MeOH (0.1% NH₃H₂O)]; B %:30%, isocratic elution mode). The first eluting single stereoisomer, Compound 50 (33.35 mg, 38.25 μmol, 42% yield) was obtained as a yellow solid. LCMS [M+H]⁺=654.4 m/z. The second eluting single stereoisomer, Compound 54 (19.82 mg, 38.25 μmol, 42% yield), was obtained as a yellow solid. LCMS [M+H]⁺=654.4 m/z.

### Example 38: Preparation of N-((1S)-(4,4-difluorocyclohexyl)(3-(dimethylamino)-2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 51 and 55)

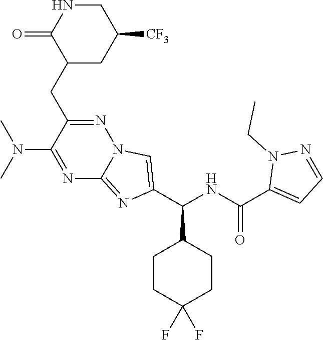 [SMILES: CCn1nccc1C(=O)N[C@H](c1cn2nc(CC3C[C@H](C(F)(F)F)CNC3=O)c(N(C)C)nc2n1)C1CCC(F)(F)CC1]

The synthesis of individual stereoisomers of N-((1 S)-(4,4-difluorocyclohexyl)(3-(dimethylamino)-2-(((5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using dimethylamine hydrochloride (1.70 eq) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, 1-(tert-butyl) 3-methyl (5S)-2-oxo-5-(trifluoromethyl)piperidine-1,3-dicarboxylate in step 7, HCl/dioxane prior to saponification in step 8, TMSI for Step 10B, and 1-ethyl-1H-pyrazole-5-carboxylic acid in step 11. The mixture of stereoisomers was purified by prep-TLC (dichloromethane:MeOH=10:1, Rf=0.50) then stereoisomers were separated by prep-SFC (column: DAICEL CHIRALCEL OD (250 mm*30 mm, 10 μm); mobile phase: [CO₂-i-PrOH (0.1% NH₃H₂O)]; B %: 45%, isocratic elution mode) and prep-HPLC (column: Waters Xbridge 150*25 mm*5 μm; mobile phase: [water (NH₄HCO₃)—acetonitrile]; gradient: 30%-60% B over 10 min). The first eluting single stereoisomer, Compound 51 (30.00 mg, 47.8 μmol, 49% yield) was obtained as a yellow solid. LCMS [M+H]⁺=612.3 m/z. The second eluting single stereoisomer, Compound 55 (28.70 mg, 45.9 μmol, 47% yield) was obtained as a yellow solid. LCMS [M+H]⁺=612.2 m/z.

### Example 39: Preparation of N-((1S)-(2-((5,5-difluoro-2-oxopiperidin-3-yl)methyl)-3-morpholinoimidazo[1,2-b][1,2,4]triazin-6-yl)(4,4-difluorocyclohexyl)methyl)-1-ethyl-1H-pyrazole-5-carboxamide (Compounds 64 and 65)

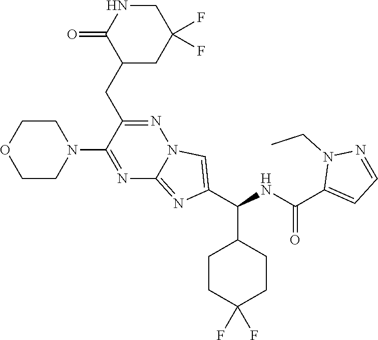 [SMILES: CCn1nccc1C(=O)N[C@H](c1cn2nc(CC3CC(F)(F)CNC3=O)c(N3CCOCC3)nc2n1)C1CCC(F)(F)CC1]

The synthesis of individual stereoisomers of N-((1S)-(2-((5,5-difluoro-2-oxopiperidin-3-yl)methyl)-3-morpholinoimidazo[1,2-b][1,2,4]triazin-6-yl)(4,4-difluorocyclohexyl)methyl)-1-ethyl-1H-pyrazole-5-carboxamide was carried out following General Scheme A, using morpholine (1.10 eq) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, methyl 5,5-difluoro-2-oxopiperidine-3-carboxylate in step 7, TMSI for Step 10B, and 1-ethyl-1H-pyrazole-5-carboxylic acid in step 11. The mixture of stereoisomers was purified by prep-TLC (dichloromethane:MeOH=10:1) then stereoisomers were separated by prep-SFC (column: REGIS (S, S) WHELK-O1 (250 mm*25 mm, 10 um); mobile phase: [CO₂-EtOH (0.1% NH₃H₂O)]; B %:50%, isocratic elution mode, Rt=1.181, 1.350 min). The first eluting single stereoisomer, Compound 64 (22.65 mg, 36.0 μmol, 28% yield), was obtained as a yellow solid. LCMS [M+H]⁺=622.4 m/z. The second eluting single stereoisomer, Compound 65 (16.16 mg, 24.9 μmol, 19% yield), was obtained as a yellow solid. LCMS [M+H]⁺=622.3 m/z.

### Example 40: Preparation of N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (Compounds 79 and 80)

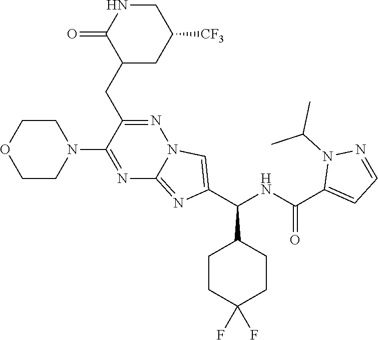 [SMILES: CC(C)n1nccc1C(=O)N[C@H](c1cn2nc(CC3C[C@@H](C(F)(F)F)CNC3=O)c(N3CCOCC3)nc2n1)C1CCC(F)(F)CC1]

The synthesis of individual stereoisomers of N-((1 S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide was carried out following General Scheme A, using morpholine (1.10 eq) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, 1-(tert-butyl) 3-methyl (5R)-2-oxo-5-(trifluoromethyl)piperidine-1,3-dicarboxylate in step 7, HCl/dioxane prior to saponification in step 8, TMSI for Step 10B, and 1-isopropyl-1H-pyrazole-5-carboxylic acid in step 11. The mixture of stereoisomers was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-acetonitrile/EtOH (0.1% NH₃H₂O)]; B %:60%, isocratic elution mode, Rt=0.495, 1.201 min). The first eluting single stereoisomer, Compound 79 (29.7 mg, 44.0 μmol, 29% yield), was obtained as a white solid. LCMS [M+H]⁺=668.3 m/z. The second eluting single stereoisomer, Compound 80 (34.6 mg, 50.2 μmol, 33% yield), was obtained as a white solid. LCMS [M+H]⁺=668.3 m/z.

### Example 41: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compounds 81 and 82)

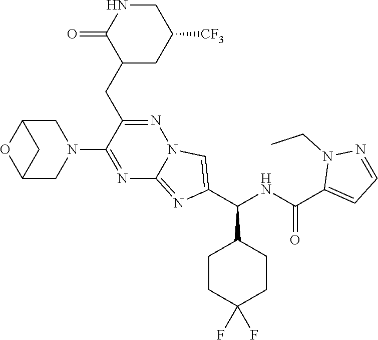 [SMILES: CCn1nccc1C(=O)N[C@H](c1cn2nc(CC3C[C@@H](C(F)(F)F)CNC3=O)c(N3CC4CC(C3)O4)nc2n1)C1CCC(F)(F)CC1]

The synthesis of individual stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl (S′)-(3-bromo-1-(4,4-difluorocyclohexyl)-2-oxopropyl)carbamate (1.10 eq) in Step 4, isobutyl chloroformate (3.00 eq) for Step 5A, MsCl in Step 6A, methyl (5R)-2-oxo-5-(trifluoromethyl)piperidine-3-carboxylate (0.33 eq) in step 7, Pd/C (10.0% purity) for Step 10A, and 1-ethyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was purified by prep-TLC (15:1 dichloromethane/MeOH) and individual stereoisomers were subsequently separated by prep-SFC (column: DAICEL CHIRALCEL OD (250 mm*30 mm, 10 μm); mobile phase: [CO₂-iPrOH/acetonitrile]; B %: 35%, isocratic elution mode). The first eluting single stereoisomer, Compound 81 (3.63 mg, 4.95 μmol, 11% yield), was obtained as a green solid. LCMS [M+H]⁺=666.3 m/z. The second eluting single stereoisomer, Compound 82 (2.45 mg, 3.53 μmol, 8% yield), was obtained as a green solid. LCMS [M+H]⁺=666.3 m/z.

### Example 42: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-ethyl-1H-pyrazole-5-carboxamide (Compounds 92 and 93)

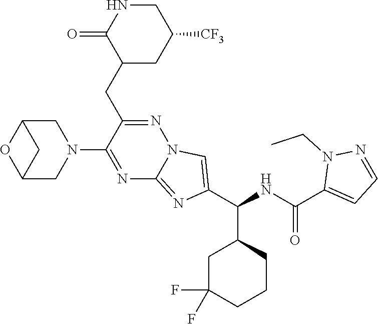 [SMILES: CCn1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]

The synthesis of individual stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-ethyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl ((S)-3-bromo-1-((S)-3,3-difluorocyclohexyl)-2-oxopropyl)carbamate (0.70 eq) in Step 4, CDI (3.00 eq) for Step 5B, MsCl for Step 6A, methyl (5R)-2-oxo-5-(trifluoromethyl)piperidine-3-carboxylate (2.00 eq) in step 7, Pd/C (10.0% purity) for Step 10A, and 1-ethyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD-H (250 mm*30 mm, 5 μm); mobile phase: [CO₂-EtOH (0.1% NH₃H₂O)]; B %:35%, isocratic elution mode) and prep-SFC again (column: REGIS(S,S)WHELK-O1 (250 mm*25 mm, 10 μm); mobile phase: [CO₂-i-PrOH/acetonitrile]; B %:45%, isocratic elution mode). The first eluting single stereoisomer, Compound 92 (15.0 mg, 21.44 μmol, 29% yield), was obtained as a yellow solid. LCMS [M+H]⁺=666.3 m/z. The second eluting single stereoisomer, Compound 93 (15.0 mg, 21.44 μmol, 29% yield), was obtained as a yellow solid. LCMS [M+H]⁺=666.3 m/z.

### Example 43: Preparation of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compounds 83 and 84)

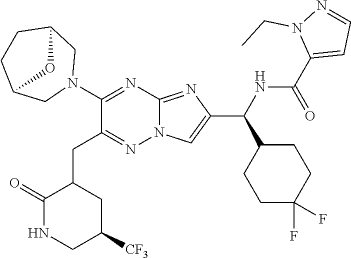 [SMILES: CCn1nccc1C(=O)N[C@H](c1cn2nc(CC3C[C@@H](C(F)(F)F)CNC3=O)c(N3C[C@H]4CC[C@@H](C3)O4)nc2n1)C1CCC(F)(F)CC1]

The synthesis of individual stereoisomers of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was carried out following General Scheme A, using 8-oxa-3-azabicyclo[3.2.1]octane (1.10 eq, HCl salt) in Step 1, benzyl (S)-(3-bromo-1-(4,4-difluorocyclohexyl)-2-oxopropyl)carbamate (1.10 eq) in Step 4, CDI (3.00 eq) for Step 5B, thionyl chloride (3.00 eq) in Step 6B, methyl (5R)-2-oxo-5-(trifluoromethyl)piperidine-3-carboxylate (1.00 eq) in step 7, Pd/C (10.0% purity) for Step 10A, and 1-ethyl-1H-pyrazole-5-carboxylic acid in step 11. The mixture of stereoisomers of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (25 0 mm*30 mm, 10 μm); mobile phase: [CO2-i-PrOH (0.1% NH₃H₂O)]; B %: 45%, isocratic elution mode, Rt=1.806, 2.167). The first eluting single stereoisomer, Compound 83 (45.37 mg, 64.8 μmol, 36% yield), was obtained as a yellow solid. LCMS [M+H]⁺=680.3 m/z. The second eluting single stereoisomer, Compound 84 (26.70 mg, 38.5 μmol, 21% yield), was obtained as a yellow solid. LCMS [M+H]⁺=680.3 m/z.

### Example 44: Preparation of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (Compounds 104 and 105)

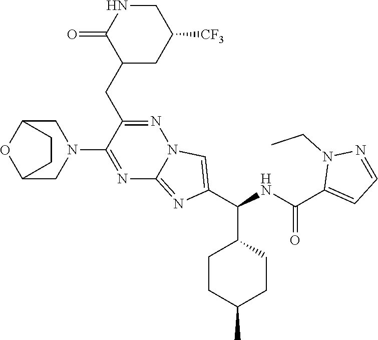 [SMILES: CCn1nccc1C(=O)N[C@H](c1cn2nc(CC3C[C@@H](C(F)(F)F)CNC3=O)c(N3CC4CCC(C3)O4)nc2n1)[C@H]1CC[C@H](C)CC1]

The synthesis of individual stereoisomers of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was carried out following General Scheme A, using 8-oxa-3-azabicyclo[3.2.1]octane (1.10 eq, HCl salt) in Step 1, benzyl ((S)-3-bromo-1-((1r,4S)-4-methylcyclohexyl)-2-oxopropyl)carbamate (1.20 eq) in Step 4, CDI (3.00 eq) for Step 5B, thionyl chloride (3.00 eq) in Step 6B, methyl (5R)-2-oxo-5-(trifluoromethyl)piperidine-3-carboxylate (1.20 eq) in step 7, Pd/C (10.0% purity) for Step 10A, and 1-ethyl-1H-pyrazole-5-carboxylic acid in step 11. The mixture of stereoisomers of N-((1S)-(3-(8-oxa-3-azabicyclo[3.2.1]octan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO2-EtOH (0.1% NH₃H₂O)]; B %: 45%, isocratic elution mode) to give two peaks. The first eluting single stereoisomer was further purified by prep-TLC (SiO2, Petroleum ether:ethyl acetate=0:1) to give Compound 104 (16.36 mg, 23.2 μmol, 31% yield), obtained as a yellow solid. LCMS [M+H]⁺=658.3 m/z. The second eluting single stereoisomer, Compound 105 (18.79 mg, 27.8 μmol, 37% yield), was obtained as a yellow solid. LCMS [M+H]⁺=658.3 m/z.

### Example 45: Preparation of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compounds 85 and 86)

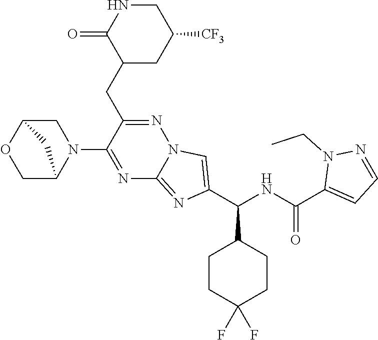 [SMILES: CCn1nccc1C(=O)N[C@H](c1cn2nc(CC3C[C@@H](C(F)(F)F)CNC3=O)c(N3C[C@H]4C[C@@H]3CO4)nc2n1)C1CCC(F)(F)CC1]

The synthesis of individual stereoisomers of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was carried out following General Scheme A, using (1R,4R)-2-oxa-5-azabicyclo[2.2.1]heptane (1.10 eq, HCl salt) in Step 1, benzyl (S)-(3-bromo-1-(4,4-difluorocyclohexyl)-2-oxopropyl)carbamate (1.00 eq) in Step 4, NaBH₄/CaCl₂ in Step 5C, thionyl chloride in Step 6B, 1-(tert-butyl) 3-methyl (5R)-2-oxo-5-(trifluoromethyl)piperidine-1,3-dicarboxylate in step 7, HCl/dioxane prior to saponification in step 8, TMSI for Step 10B, and 1-ethyl-1H-pyrazole-5-carboxylic acid in step 11. The mixture of stereoisomers of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OX (250 mm*30 mm, 10 μm); mobile phase: [CO₂-EtOH (0.1% NH₃H₂O)]; B %:35%, isocratic elution mode). The first eluting single stereoisomer, Compound 85 (22.57 mg, 32.2 μmol, 31% yield), was obtained as a yellow solid. LCMS [M+H]⁺=666.4 m/z. The second eluting single stereoisomer was further purified by prep-HPLC (column: Phenomenex luna C18 150*25 mm*10 μm; mobile phase: [water (FA)—acetonitrile]; gradient:15%-45% B over 10 min) to afford Compound 86 (14.41 mg, 21.0 μmol, 20% yield) as a yellow solid. LCMS [M+H]⁺=666.4 m/z.

### Example 46: Preparation of N-((1S)—((S)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 90 and 91)

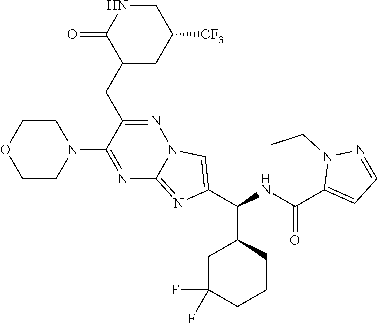 [SMILES: CCn1nccc1C(=O)N[C@H](c1cn2nc(CC3C[C@@H](C(F)(F)F)CNC3=O)c(N3CCOCC3)nc2n1)[C@H]1CCCC(F)(F)C1]

The synthesis of individual stereoisomers of N-((1S)—((S)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using morpholine (1.00 eq, freebase) in Step 1, benzyl (S)-(3-bromo-1-(4,4-difluorocyclohexyl)-2-oxopropyl)carbamate (0.90 eq) in Step 4, CDI (3.00 eq) for Step 5B, thionyl chloride (3.00 eq) in Step 6B, methyl (5R)-2-oxo-5-(trifluoromethyl)piperidine-3-carboxylate (1.10 eq) in step 7, TMSI for Step 10B, and 1-ethyl-1H-pyrazole-5-carboxylic acid in step 11. The mixture of stereoisomers of N-((1S)—((S)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-MeOH (0.1% NH₃H₂O)]; B %: 55%, isocratic elution mode, Rt=1.708, 2.373). The first eluting single stereoisomer, Compound 90 (25.7 mg, 38.1 μmol, 38% yield), was obtained as a yellow solid. LCMS [M+H]⁺=654.3 m/z. The second eluting single stereoisomer, Compound 91 (21.7 mg, 32.6 μmol, 32% yield), was obtained as a yellow solid. LCMS [M+H]⁺=654.3 m/z.

### Example 47: Preparation of N-((1S)—((R)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 94 and 95)

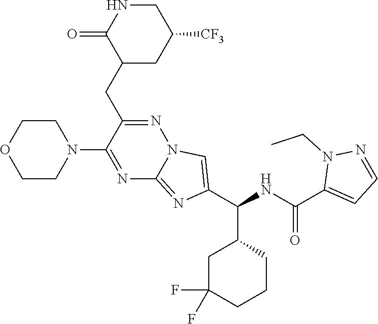 [SMILES: CCn1nccc1C(=O)N[C@H](c1cn2nc(CC3C[C@@H](C(F)(F)F)CNC3=O)c(N3CCOCC3)nc2n1)[C@@H]1CCCC(F)(F)C1]

The synthesis of individual stereoisomers of N-((1S)—((R)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out in the same manner as that of N-((1S)—((S)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, with the only changes being the use of benzyl ((S)-3-bromo-1-((R)-3,3-difluorocyclohexyl)-2-oxopropyl)carbamate as the bromoketone in this synthesis and the final purification. Individual stereoisomers of N-((1S)—((R)-3,3-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide were separated by prep-SFC (column: DAICEL CHIRALCEL OD (250 mm*30 mm, 10 μm); mobile phase: [CO2-MeOH (0.1% NH₃H₂O)]; B %:65%, isocratic elution mode). The first eluting single stereoisomer, Compound 94 (27.67 mg, 39.75 μmol, 37% yield), was obtained as a yellow solid. LCMS [M+H]⁺=654.3 m/z. The second eluting single stereoisomer, Compound 95 (18.56 mg, 28.34 μmol, 26% yield), was obtained as a yellow solid. LCMS [M+H]⁺=654.3 m/z.

### Example 48: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-ethyl-1H-pyrazole-5-carboxamide (Compounds 96 and 97)

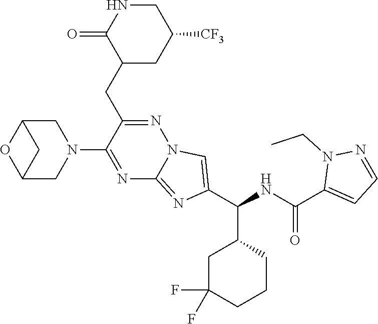 [SMILES: CCn1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]

The synthesis of individual stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-ethyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl ((S)-3-bromo-1-((R)-3,3-difluorocyclohexyl)-2-oxopropyl)carbamate (1.30 eq) in Step 4, isobutyl chloroformate (2.00 eq) for Step 5A, MsCl for Step 6A, methyl (5R)-2-oxo-5-(trifluoromethyl)piperidine-3-carboxylate (1.50 eq) in step 7, Pd/C (10.0% purity) for Step 10A, and 1-ethyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-i-PrOH]; B %: 40%, isocratic elution mode). The first eluting single stereoisomer, Compound 96 (16.6 mg, 24.2 μmol, 46% yield), was obtained as a yellow solid. LCMS [M+H]⁺=666.2 m/z. The second eluting single stereoisomer, Compound 97 (12.1 mg, 17.4 μmol, 33% yield), was obtained as a yellow solid. LCMS [M+H]⁺=666.3 m/z.

### Example 49: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-isopropyl-1H-pyrazole-5-carboxamide (Compounds 129 and 131)

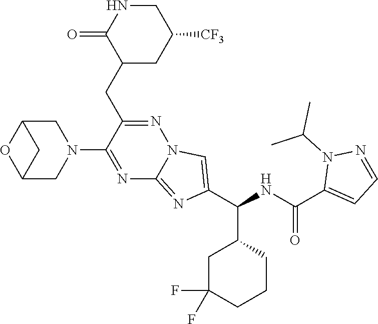 [SMILES: CC(C)n1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]

The synthesis of individual stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-isopropyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl ((S)-3-bromo-1-((R)-3,3-difluorocyclohexyl)-2-oxopropyl)carbamate (1.30 eq) in Step 4, isobutyl chloroformate (2.00 eq) for Step 5A, MsCl for Step 6A, methyl (5R)-2-oxo-5-(trifluoromethyl)piperidine-3-carboxylate (1.50 eq) in step 7, Pd/C (10.0% purity) for Step 10A, and 1-isopropyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((R)-3,3-difluorocyclohexyl)methyl)-1-isopropyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-i-PrOH]; B %: 40%, isocratic elution mode). The first eluting single stereoisomer, Compound 129 (28.4 mg, 40.0 μmol, 38% yield), was obtained as a yellow solid. LCMS [M+H]⁺=680.3 m/z. The second eluting single stereoisomer, Compound 131 (24.4 mg, 35.1 μmol, 34% yield), was obtained as a yellow solid. LCMS [M+H]⁺=680.3 m/z.

### Example 50: Preparation of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compounds 98 and 99)

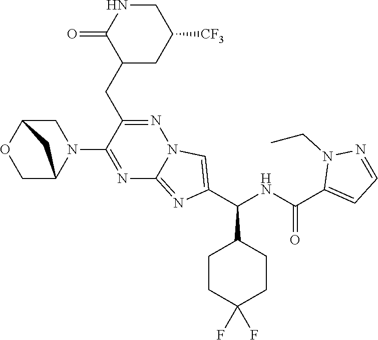 [SMILES: CCn1nccc1C(=O)N[C@H](c1cn2nc(CC3C[C@@H](C(F)(F)F)CNC3=O)c(N3C[C@@H]4C[C@H]3CO4)nc2n1)C1CCC(F)(F)CC1]

The synthesis of individual stereoisomers of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was carried out following General Scheme A, using (1S,4S)-2-oxa-5-azabicyclo[2.2.1]heptane (1.10 eq, HCl salt) in Step 1, benzyl (S′)-(3-bromo-1-(4,4-difluorocyclohexyl)-2-oxopropyl)carbamate (1.00 eq) in Step 4, NaBH₄/CaCl₂ in Step 5C, thionyl chloride in Step 6B, 1-(tert-butyl) 3-methyl (5R)-2-oxo-5-(trifluoromethyl)piperidine-1,3-dicarboxylate in step 7, HCl/dioxane prior to saponification in step 8, TMSI for Step 10B, and 1-ethyl-1H-pyrazole-5-carboxylic acid in step 11. The mixture of stereoisomers of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was purified by prep-TLC (10:1 dichloromethane/MeOH) and individual stereoisomers were subsequently separated by prep-SFC (basic condition, column: DAICEL CHIRALCEL OJ (250 mm*30 mm, 10 um); mobile phase: [CO₂-EtOH (0.1% NH₃H₂O)]; B %: 30%, isocratic elution mode). The first eluting single stereoisomer, Compound 98 (26.47 mg, 38.4 μmol, 28% yield), was obtained as a yellow solid. LCMS [M+H]⁺=666.3 m/z. The second eluting single stereoisomer, Compound 99 (25.83 mg, 37.9 μmol, 28% yield) was obtained as a yellow solid. LCMS [M+H]⁺=666.4 m/z.

### Example 51: Preparation of 1-isopropyl-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)-1H-pyrazole-5-carboxamide (Compounds 100 and 101)

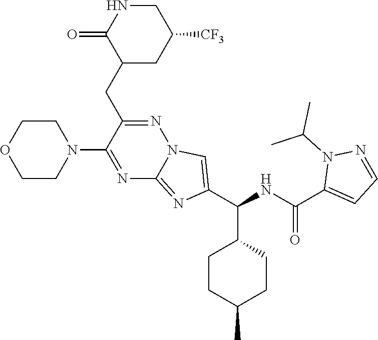 [SMILES: CC(C)n1nccc1C(=O)N[C@H](c1cn2nc(CC3C[C@@H](C(F)(F)F)CNC3=O)c(N3CCOCC3)nc2n1)[C@H]1CC[C@H](C)CC1]

The synthesis of individual stereoisomers of 1-isopropyl-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)-1H-pyrazole-5-carboxamide was carried out following General Scheme A, using morpholine (1.10 eq, freebase) in Step 1, benzyl ((S)-3-bromo-1-((1r,4S)-4-methylcyclohexyl)-2-oxopropyl)carbamate (1.00 eq) in Step 4, NaBH₄/CaCl₂ in Step 5C, thionyl chloride in Step 6B, methyl (5R)-2-oxo-5-(trifluoromethyl)piperidine-3-carboxylate in step 7, TMSI for Step 10B, and 1-isopropyl-1H-pyrazole-5-carboxylic acid in step 11. The mixture of stereoisomers of 1-isopropyl-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-EtOH (0.1% NH₃H₂O)]; B %:60%, isocratic elution mode). The first eluting single stereoisomer, Compound 100 (46.2 mg, 71.5 μmol, 46% yield), was obtained as a yellow solid. LCMS [M+H]⁺=646.6 m/z. The second eluting single stereoisomer, Compound 101 (29.4 mg, 45.6 μmol, 29% yield) was obtained as a yellow solid. LCMS [M+H]⁺=646.5 m/z.

### Example 52: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (Compounds 102 and 103)

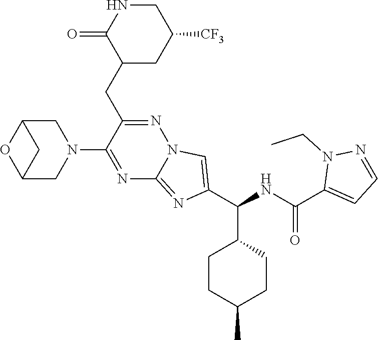 [SMILES: CCn1nccc1C(=O)N[C@H](c1cn2nc(CC3C[C@@H](C(F)(F)F)CNC3=O)c(N3CC4CC(C3)O4)nc2n1)[C@H]1CC[C@H](C)CC1]

The synthesis of individual stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl ((S)-3-bromo-1-((1r,4S)-4-methylcyclohexyl)-2-oxopropyl)carbamate (1.10 eq) in Step 4, isobutyl chloroformate (4.00 eq) for Step 5A, MsCl (6.00 eq) in Step 6A, methyl (5R)-2-oxo-5-(trifluoromethyl)piperidine-3-carboxylate (2.00 eq) in step 7, Pd/C (10.0% purity) for Step 10A, and 1-ethyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-i-PrOH/acetonitrile]; B %: 35%, isocratic elution mode). The first eluting single stereoisomer, Compound 102 (28.0 mg, 56.2 μmol, 52% yield), was obtained as a yellow solid. LCMS [M+H]⁺=644.4 m/z. The second eluting single stereoisomer, Compound 103 (14.6 mg, 30.6 μmol, 28% yield), was obtained as a yellow solid. LCMS [M+H]⁺=644.4 m/z.

### Example 53: Preparation of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (Compounds 106 and 107)

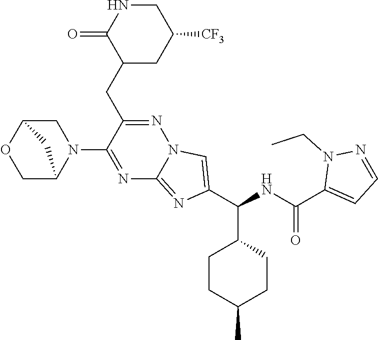 [SMILES: CCn1nccc1C(=O)N[C@H](c1cn2nc(CC3C[C@@H](C(F)(F)F)CNC3=O)c(N3C[C@H]4C[C@@H]3CO4)nc2n1)[C@H]1CC[C@H](C)CC1]

The synthesis of individual stereoisomers of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was carried out following General Scheme A, using (1R,4R)-2-oxa-5-azabicyclo[2.2.1]heptane (1.10 eq, HCl salt) in Step 1, benzyl ((S)-3-bromo-1-((1r,4S)-4-methylcyclohexyl)-2-oxopropyl)carbamate (1.15 eq) in Step 4, NaBH₄/CaCl₂ for Step 5C, thionyl chloride (3.00 eq) in Step 6B, methyl (5R)-2-oxo-5-(trifluoromethyl)piperidine-3-carboxylate (1.10 eq) in step 7, Pd/C (10.0% purity) for Step 10A, and 1-ethyl-1H-pyrazole-5-carboxylic acid in step 11. The mixture of stereoisomers of N-((1S)-(3-((1R,4R)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OX (250 mm*30 mm, 10 μm); mobile phase: [CO₂-MeOH (0.1% NH₃H₂O)]; B %: 50%, isocratic elution mode). The first eluting single stereoisomer, Compound 106 (22.39 mg, 33.2 μmol, 22% yield), was obtained as a yellow solid. LCMS [M+H]+=644.4 m/z. The second eluting single stereoisomer, Compound 107 (28.40 mg, 40.8 μmol, 27% yield), was obtained as a yellow solid. LCMS [M+H]+=644.4 m/z.

### Example 54: Preparation of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (Compounds 108 and 109)

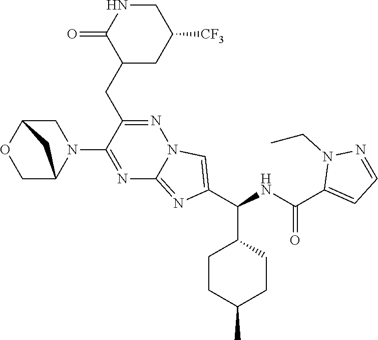 [SMILES: CCn1nccc1C(=O)N[C@H](c1cn2nc(CC3C[C@@H](C(F)(F)F)CNC3=O)c(N3C[C@@H]4C[C@H]3CO4)nc2n1)[C@H]1CC[C@H](C)CC1]

The synthesis of individual stereoisomers of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was carried out following General Scheme A, using (1S,4S)-2-oxa-5-azabicyclo[2.2.1]heptane (1.10 eq, HCl salt) in Step 1, benzyl ((S)-3-bromo-1-((1r,4S)-4-methylcyclohexyl)-2-oxopropyl)carbamate (1.00 eq) in Step 4, NaBH₄/CaCl₂ for Step 5C, thionyl chloride (5.00 eq) in Step 6B, 1-(tert-butyl) 3-methyl (5R)-2-oxo-5-(trifluoromethyl)piperidine-1,3-dicarboxylate (1.10 eq) in step 7, HCl/dioxane prior to saponification in step 8, Pd/C (10.0% purity) for Step 10A, and 1-ethyl-1H-pyrazole-5-carboxylic acid in step 11. The mixture of stereoisomers of N-((1S)-(3-((1S,4S)-2-oxa-5-azabicyclo[2.2.1]heptan-5-yl)-2-(((5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was purified by prep-TLC (10:1 dichloromethane/MeOH) and individual stereoisomers were subsequently separated by prep-SFC (column: REGIS (S,S) WHELK-O1 (250 mm*25 mm, 10 μm); mobile phase: [CO₂-acetonitrile/MeOH (0.1% NH₃H₂O)]; B %: 50%, isocratic elution mode).

The first eluting single stereoisomer, Compound 108 (34.6 mg, 52.2 μmol, 42% yield), was obtained as a yellow solid. LCMS [M+H]+=644.3 m/z. The second eluting single stereoisomer, Compound 109 (19.63 mg, 28.88 μmol, 23% yield), was obtained as a yellow solid. LCMS [M+H]+=644.3 m/z.

### Example 55: Preparation of N-((1S)-2,2-dicyclopropyl-1-(3-morpholino-2-(((5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (Compounds 111 and 112)

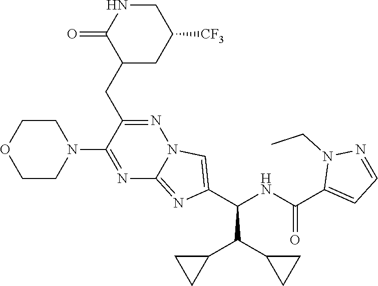 [SMILES: CCn1nccc1C(=O)N[C@H](c1cn2nc(CC3C[C@@H](C(F)(F)F)CNC3=O)c(N3CCOCC3)nc2n1)C(C1CC1)C1CC1]

The synthesis of individual stereoisomers of N-((1 S)-2,2-dicyclopropyl-1-(3-morpholino-2-(((5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide was carried out following General Scheme A, using morpholine (1.00 eq, freebase) in Step 1, benzyl (S)-(4-bromo-1,1-dicyclopropyl-3-oxobutan-2-yl)carbamate (1.20 eq) in Step 4, NaBH₄/CaCl₂ for Step 5C, thionyl chloride (3.00 eq) in Step 6B, 1-(tert-butyl) 3-methyl (5R)-2-oxo-5-(trifluoromethyl)piperidine-1,3-dicarboxylate (1.05 eq) in step 7, HCl/dioxane prior to saponification in step 8, TMSI for Step 10B, and 1-ethyl-1H-pyrazole-5-carboxylic acid in step 11. The mixture of stereoisomers of N-((1 S)-2,2-dicyclopropyl-1-(3-morpholino-2-(((5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-i-PrOH (0.1% NH₃H₂O)]; B %: 65%, isocratic elution mode). The first eluting single stereoisomer, Compound 111 (27.4 mg, 41.7 μmol, 29% yield), was obtained as a yellow solid. LCMS [M+H]⁺=630.3 m/z. The second eluting single stereoisomer, Compound 112 (26.0 mg, 40.4 μmol, 28% yield), was obtained as a yellow solid. LCMS [M+H]⁺=630.4 m/z.

### Example 56: Preparation of N-((1S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-3-(2,2-difluorocyclopropyl)isoxazole-4-carboxamide (Compounds 113, 114, 115, and 116)

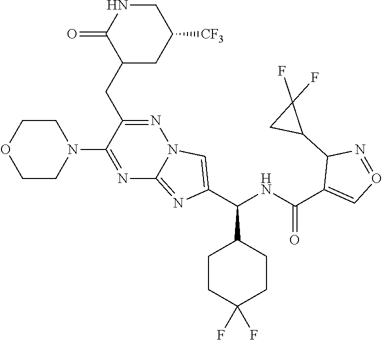

The synthesis of individual stereoisomers of N-((1 S)-(4,4-difluorocyclohexyl)(3-morpholino-2-(((5R)-2-oxo-5-(trifluoromethyl)piperidin-3-yl)methyl)imidazo[1,2-b][1,2,4]triazin-6-yl)methyl)-3-(2,2-difluorocyclopropyl)isoxazole-4-carboxamide was carried out following General Scheme A, using morpholine (1.10 eq) in Step 1, benzyl (S)-(3-bromo-1-(4,4-difluorocyclohexyl)-2-oxopropyl)carbamate (1.20 eq) in Step 4, NaBH₄/CaCl₂ in Step 5C, thionyl chloride in Step 6B, 1-(tert-butyl) 3-methyl (5R)-2-oxo-5-(trifluoromethyl)piperidine-1,3-dicarboxylate in step 7, HCl/dioxane prior to saponification in step 8, TMSI for Step 10B, and 3-(2,2-difluorocyclopropyl)isoxazole-4-carboxylic acid in step 11. The mixture of 4 stereoisomers was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-MeOH (0.1% NH₃H₂O)]; B %: 55%, isocratic elution mode) to give two peaks, each with two single stereoisomers. Peak 1 was further purified by prep-SFC (column: DAICEL CHIRALPAK IG (250 mm*30 mm, 10 μm); mobile phase: [CO₂-EtOH (0.1% NH₃H₂O)]; B %: 40%, isocratic elution mode) to give two individual stereoisomers. The first eluting single stereoisomer, Compound 113 (58.47 mg, 78.2 μmol, 22% yield), was obtained as a yellow solid. LCMS [M+H]⁺=703.2 m/z. The second eluting single stereoisomer, Compound 114 (44.85 mg, 62.6 μmol, 18% yield), was obtained as a yellow solid. LCMS [M+H]⁺=703.3 m/z. Peak 2 from the initial SFC separation was further purified by prep-SFC (column: DAICEL CHIRALPAK IG (250 mm*30 mm, 10 μm); mobile phase: [CO₂-EtOH (0.1% NH₃H₂O)]; B %: 45%, isocratic elution mode). The first eluting single stereoisomer from this separation, Compound 115 (32.93 mg, 46.4 μmol, 13% yield), was obtained as a yellow solid. LCMS [M+H]⁺=703.2 m/z. The second eluting single stereoisomer from this separation, Compound 116 (35.66 mg, 49.6 μmol, 14% yield), was obtained as a yellow solid. LCMS [M+H]⁺=703.2 m/z.

### Example 57: Preparation of N—((S)—((S)-3,3-difluorocyclohexyl)(3-(dimethylamino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 117)

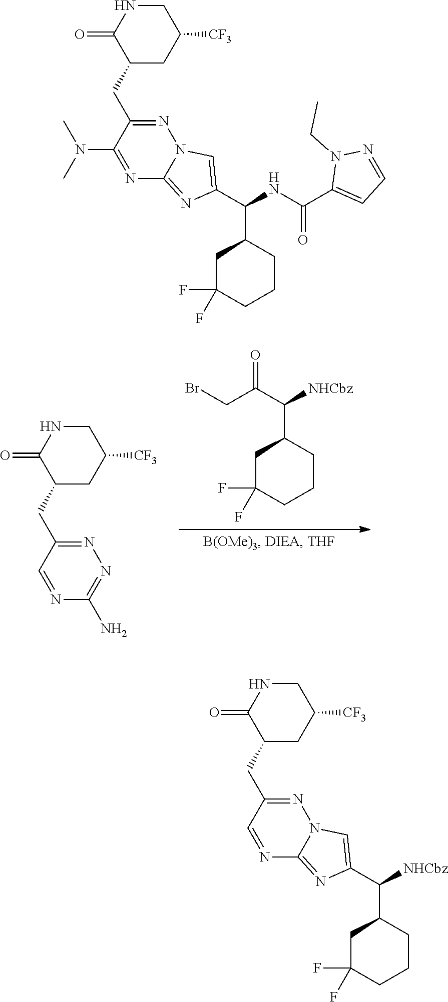 [SMILES: CCn1nccc1C(=O)N[C@H](c1cn2nc(C[C@H]3C[C@@H](C(F)(F)F)CNC3=O)c(N(C)C)nc2n1)[C@H]1CCCC(F)(F)C1 ; Nc1ncc(C[C@H]2C[C@@H](C(F)(F)F)CNC2=O)nn1 ; O=C(N[C@H](C(=O)CBr)[C@H]1CCCC(F)(F)C1)OCc1ccccc1 ; O=C(N[C@H](c1cn2nc(C[C@H]3C[C@@H](C(F)(F)F)CNC3=O)cnc2n1)[C@H]1CCCC(F)(F)C1)OCc1ccccc1]

To a solution of (3R,5R)-3-((3-amino-1,2,4-triazin-6-yl)methyl)-5-(trifluoromethyl)piperidin-2-one (400 mg, 1.45 mmol, 1.00 eq) and benzyl ((S)-3-bromo-1-((R)-3,3-difluorocyclohexyl)-2-oxopropyl)carbamate (646 mg, 1.60 mmol, 1.10 eq) in THF (6.00 mL) was added B(OMe)₃ (453 mg, 4.36 mmol, 492 μL, 3.00 eq) and DIEA (939 mg, 7.27 mmol, 1.27 mL, 5.00 eq). The mixture was stirred at 70° C. for 5 h. The mixture was diluted with H₂O (15.0 mL) and extracted with ethyl acetate (15.0 mL*3). The combined organic layers were dried over Na₂SO₄, filtered, and concentrated under reduced pressure to give a residue, which was purified by column chromatography (SiO₂, ethyl acetate:petroleum ether=1:0) to afford benzyl ((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (0.580 g, 999 μmol, 69% yield) as a yellow solid. LCMS [M+H]+=581.4 m/z.

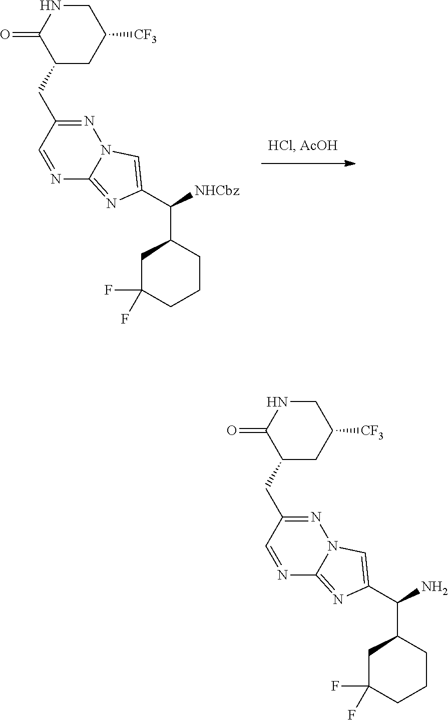 [SMILES: O=C(N[C@H](c1cn2nc(C[C@H]3C[C@@H](C(F)(F)F)CNC3=O)cnc2n1)[C@H]1CCCC(F)(F)C1)OCc1ccccc1 ; N[C@H](c1cn2nc(C[C@H]3C[C@@H](C(F)(F)F)CNC3=O)cnc2n1)[C@H]1CCCC(F)(F)C1]

Benzyl ((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (0.580 g, 999 μmol, 1.00 eq) was subjected to the conditions in General Procedure 5 (0.2 M in 10:1 aq. HCl/AcOH, 55° C., 0.5 h) to afford (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one (290 mg, 649 μmol, 65% yield) as a yellow solid. LCMS [M+H]+=447.2 m/z.

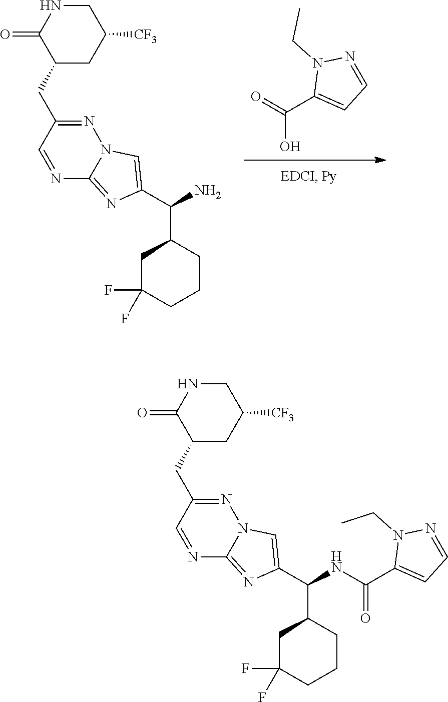

Amide coupling of (3R,5R)-3-((6-((S)-amino((S)-3,3-difluorocyclohexyl)methyl) imidazo[1,2-b][1,2,4]triazin-2-yl)methyl)-5-(trifluoromethyl)piperidin-2-one (0.290 g, 649 μmol, 1.00 eq) with 1-ethyl-1H-pyrazole-5-carboxylic acid (100 mg, 714 μmol, 1.10 eq) using EDCI (373 mg, 1.95 mmol, 3.00 eq) in pyridine (2.00 mL) was carried out following the conditions in General Procedure 6. The product, N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (260 mg, 457 μmol, 70% yield), was obtained as a white solid. LCMS [M+H]+=569.2 m/z.

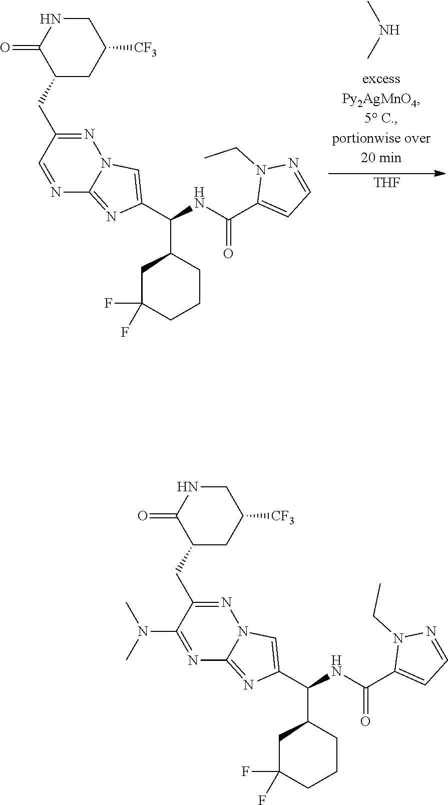

Oxidative nucleophilic substitution of hydrogen of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with dimethylamine (2 M in THF, 20.0 eq) was carried out following General Procedure 8, using bis(pyridine)silver(I) permanganate (5.00 eq) in THF (0.18 M) and purifying by prep-TLC (dichloromethane/MeOH, 10:1) to afford N—((S)—((S)-3,3-difluorocyclohexyl)(3-(dimethylamino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 117 (69% yield). LCMS [M+H]⁺=612.3 m/z.

### Example 58: Preparation of N—((S)—((S)-3,3-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 121)

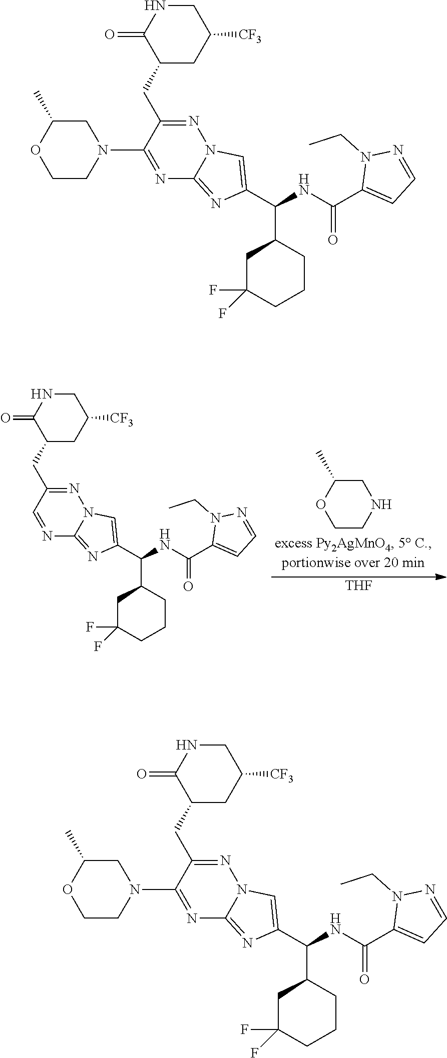

Oxidative nucleophilic substitution of hydrogen of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with (R)-2-methylmorpholine (20.0 eq) was carried out following General Procedure 8, using bis(pyridine)silver(I) permanganate (5.00 eq) in THF (0.14 M) and purifying by prep-TLC (dichloromethane/MeOH, 10:1) to afford N—((S)—((S)-3,3-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 121 (91% yield). LCMS [M+H]⁺=668.3 m/z.

### Example 59: Preparation of N—((S)—((S)-3,3-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 123)

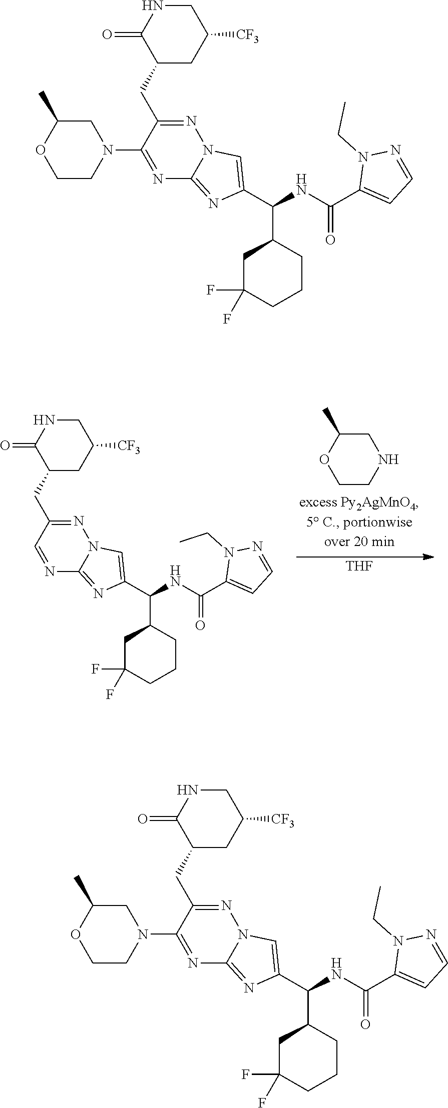

Oxidative nucleophilic substitution of hydrogen of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with (S)-2-methylmorpholine (20.0) was carried out following General Procedure 8, using bis(pyridine)silver(I) permanganate (5.00 eq) in THF (0.14 M) and purifying by prep-TLC (dichloromethane/MeOH, 10:1) to afford N—((S)—((S)-3,3-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide Compound 123 (85% yield). LCMS [M+H]⁺=668.5 m/z.

### Example 60: Preparation of 1-fluoro-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)cyclopropane-1-carboxamide (Compounds 118 and 120)

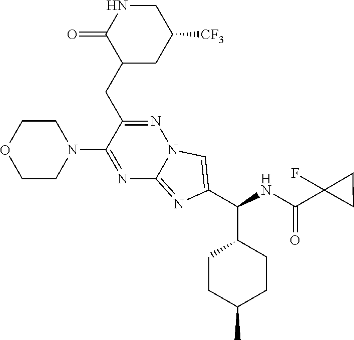 [SMILES: C[C@H]1CC[C@H]([C@H](NC(=O)C2(F)CC2)c2cn3nc(CC4C[C@@H](C(F)(F)F)CNC4=O)c(N4CCOCC4)nc3n2)CC1]

The synthesis of individual stereoisomers of 1-fluoro-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)cyclopropane-1-carboxamide was carried out in the same manner as that of 1-isopropyl-N-((1 S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)-1H-pyrazole-5-carboxamide, with the only changes being the use of 1-ethyl-1H-pyrazole-5-carboxylic acid in Step 11 of this synthesis and the final purification. Individual stereoisomers of 1-fluoro-N-((1S)-((1r,4S)-4-methylcyclohexyl)(3-morpholino-2-(((5R)-2-oxo-5-(trifluoromethyl)piperidin-3-yl)methyl)imidazo[1,2-b][1,2,4]triazin-6-yl)methyl)cyclopropane-1-carboxamide were separated by prep-SFC (column: DAICEL CHIRALCEL OX (250 mm*30 mm, 10 μm); mobile phase: [CO₂-(0.1% NH₃H₂O)]; B %:60%, isocratic elution mode). The first eluting single stereoisomer, Compound 118 (22.8 mg, 37.2 μmol, 44% yield), was obtained as a yellow solid. LCMS [M+H]⁺=596.4 m/z. The second eluting single stereoisomer, Compound 120 (23.7 mg, 37.9 μmol, 45% yield), was obtained as a yellow solid. LCMS [M+H]⁺=596.4 m/z.

### Example 61: Preparation of N—((S)—((R)-3,3-difluorocyclohexyl)(3-(dimethylamino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 119)

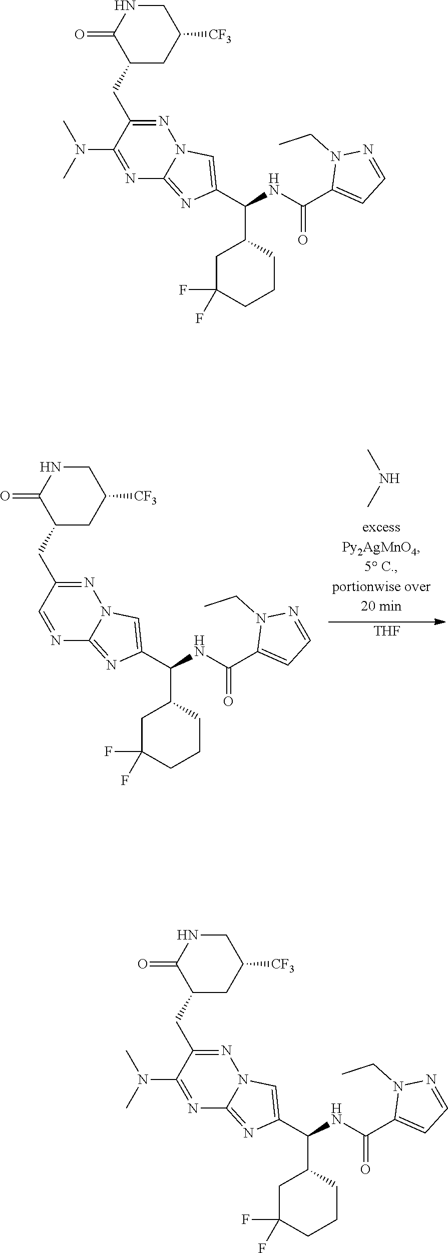

Oxidative nucleophilic substitution of hydrogen of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with dimethylamine (2 M in THF, 20.0 eq) was carried out following General Procedure 8, using bis(pyridine)silver(I) permanganate (1.50 eq) in THF (0.1 M) and purifying by prep-TLC (dichloromethane/MeOH, 10:1) to afford N—((S)—((R)-3,3-difluorocyclohexyl)(3-(dimethylamino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 119 (30% yield). LCMS [M+H]⁺=612.4 m/z.

### Example 62: Preparation of N-((1S)—((R)-3,3-difluorocyclohexyl)(3-((R)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 122 and 124)

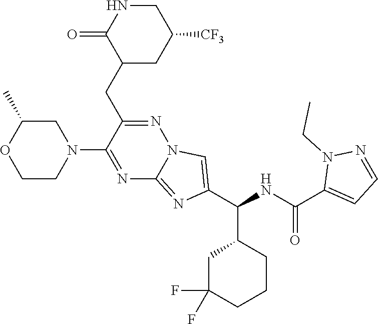 [SMILES: CCn1nccc1C(=O)N[C@H](c1cn2nc(CC3C[C@@H](C(F)(F)F)CNC3=O)c(N3CCO[C@H](C)C3)nc2n1)[C@@H]1CCCC(F)(F)C1]

The synthesis of individual stereoisomers of N-((1 S)—((R)-3,3-difluorocyclohexyl)(3-((R)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using (R)-2-methylmorpholine (1.05 eq, HCl salt) in Step 1, benzyl ((S)-3-bromo-1-((R)-3,3-difluorocyclohexyl)-2-oxopropyl)carbamate (0.80 eq) in Step 4, isobutyl chloroformate for Step 5A, thionyl chloride (3.00 eq) in Step 6B, 1-(tert-butyl) 3-methyl (5R)-2-oxo-5-(trifluoromethyl)piperidine-1,3-dicarboxylate (1.50 eq) in step 7, HCl/dioxane prior to saponification in step 8, TMSI for Step 10B, and 1-ethyl-1H-pyrazole-5-carboxylic acid in step 11. The mixture of stereoisomers of N-((1S)—((R)-3,3-difluorocyclohexyl)(3-((R)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was purified by prep-TLC (10:1 dichloromethane/MeOH) and individual stereoisomers were subsequently separated by prep-SFC (column: DAICEL CHIRALCEL OD (250 mm*30 mm, 10 μm); mobile phase: [CO₂-i-PrOH (0.1% NH₃·H₂O)]; B %:35%, isocratic elution mode) and the Peak 1 was further purified by prep-SFC (column: (S,S) WHELK-O1 (250 mm*30 mm, 10 μm); mobile phase: [CO₂-i-PrOH (0.1% NH₃·H₂O)]; B %:60%, isocratic elution mode). The first eluting single stereoisomer was further purified by prep-SFC (column: (S,S) WHELK-O1 (250 mm*30 mm, 10 μm); mobile phase: [CO₂-i-PrOH (0.1% NH₃·H₂O)]; B %:60%, isocratic elution mode) to afford Compound 122 (38.32 mg, 56.4 μmol, 25% yield) as a yellow solid. LCMS [M+H]⁺=668.4 m/z. The second eluting single stereoisomer was further purified by prep-SFC (column: (S,S) WHELK-O1 (250 mm*30 mm, 10 μm); mobile phase: [CO₂-i-PrOH (0.1% NH₃·H₂O)]; B %:60%, isocratic elution mode) to afford Compound 124 (27.29 mg, 40.3 μmol, 18% yield) as a yellow solid. LCMS [M+H]⁺=668.4 m/z.

### Example 63: Preparation of N-((1S)—((R)-3,3-difluorocyclohexyl)(3-((S)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 207 and 208)

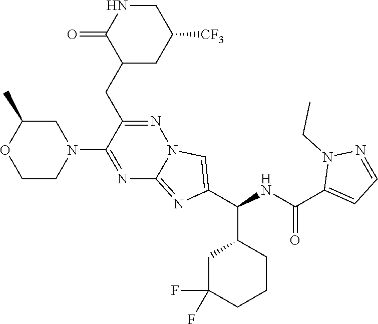 [SMILES: CCn1nccc1C(=O)N[C@H](c1cn2nc(CC3C[C@@H](C(F)(F)F)CNC3=O)c(N3CCO[C@@H](C)C3)nc2n1)[C@@H]1CCCC(F)(F)C1]

The synthesis of individual stereoisomers of N-((1 S)—((R)-3,3-difluorocyclohexyl)(3-((S)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was carried out following General Scheme A, using (S)-2-methylmorpholine (1.05 eq, HCl salt) in Step 1, benzyl ((S)-3-bromo-1-((R)-3,3-difluorocyclohexyl)-2-oxopropyl)carbamate (0.80 eq) in Step 4, isobutyl chloroformate for Step 5A, thionyl chloride (3.00 eq) in Step 6B, 1-(tert-butyl) 3-methyl (5R)-2-oxo-5-(trifluoromethyl)piperidine-1,3-dicarboxylate (1.50 eq) in step 7, HCl/dioxane prior to saponification in step 8, TMSI for Step 10B, and 1-ethyl-1H-pyrazole-5-carboxylic acid in step 11. The mixture of stereoisomers of N-((1 S)—((R)-3,3-difluorocyclohexyl)(3-((S)-2-methylmorpholino)-2-(((5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was purified by prep-TLC (10:1 dichloromethane/MeOH) and individual stereoisomers were subsequently separated by prep-SFC (column: REGIS (S, S) WHELK-O1 (250 mm*25 mm, 10 μm); mobile phase: [CO₂-EtOH (0.1% NH₃H₂O)]; B %:40%, isocratic elution mode). The first eluting single stereoisomer, Compound 207 (15.0 mg, 22.4 μmol, 25% yield), was obtained as a white solid. LCMS [M+H]⁺=668.4 m/z. The second eluting single stereoisomer, Compound 208 (5.00 mg, 7.49 μmol, 8% yield), was obtained as a white solid. LCMS [M+H]⁺=668.4 m/z.

### Example 64: Preparation of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-isopropyl-1H-pyrazole-5-carboxamide (Compounds 128 and 130)

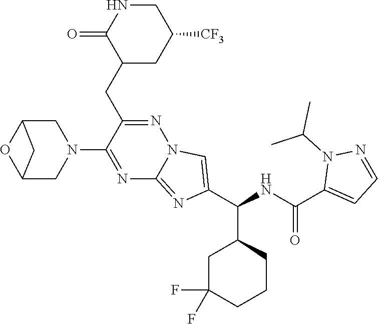 [SMILES: CC(C)n1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]

The synthesis of individual stereoisomers of N-((1 S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-isopropyl-1H-pyrazole-5-carboxamide was carried out following General Scheme A, using 6-oxa-3-azabicyclo[3.1.1]heptane (1.00 eq, p-toluenesulfonic acid salt) in Step 1, benzyl ((S)-3-bromo-1-((R)-3,3-difluorocyclohexyl)-2-oxopropyl)carbamate (1.10 eq) in Step 4, isobutyl chloroformate (4.00 eq) for Step 5A, MsCl (4.00 eq) in Step 6A, methyl (5R)-2-oxo-5-(trifluoromethyl)piperidine-3-carboxylate (1.50 eq) in step 7, Pd/C (10.0% purity) for Step 10A, and 1-isopropyl-1H-pyrazole-5-carboxylic acid in step 11. The mixture of stereoisomers of N-((1S)-(3-(6-oxa-3-azabicyclo[3.1.1]heptan-3-yl)-2-(((5R)-2-oxo-5-(trifluoromethyl)piperidin-3-yl)methyl)imidazo[1,2-b][1,2,4]triazin-6-yl)((S)-3,3-difluorocyclohexyl)methyl)-1-isopropyl-1H-pyrazole-5-carboxamide was purified by prep-TLC (10:1 dichloromethane/MeOH) and individual stereoisomers were subsequently separated by prep-SFC (column: DAICEL CHTRALCEL OD (250 mm*30 mm, 10 μm); mobile phase: [CO₂-i-PrOH]; B %: 40%, isocratic elution mode). The first eluting single stereoisomer, Compound 128 (7.82 mg, 10.72 μmol, 8% yield), was obtained as a yellow solid. LCMS [M+H]⁺=680.4 m/z. The second eluting single stereoisomer, Compound 130 (7.07 mg, 9.46 μmol, 7.34% yield), was obtained as a yellow solid. LCMS [M+H]⁺=680.4 m/z.

### Example 65: Preparation of N—((S)-(3-(cyclopropyl(methyl)amino)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 143)

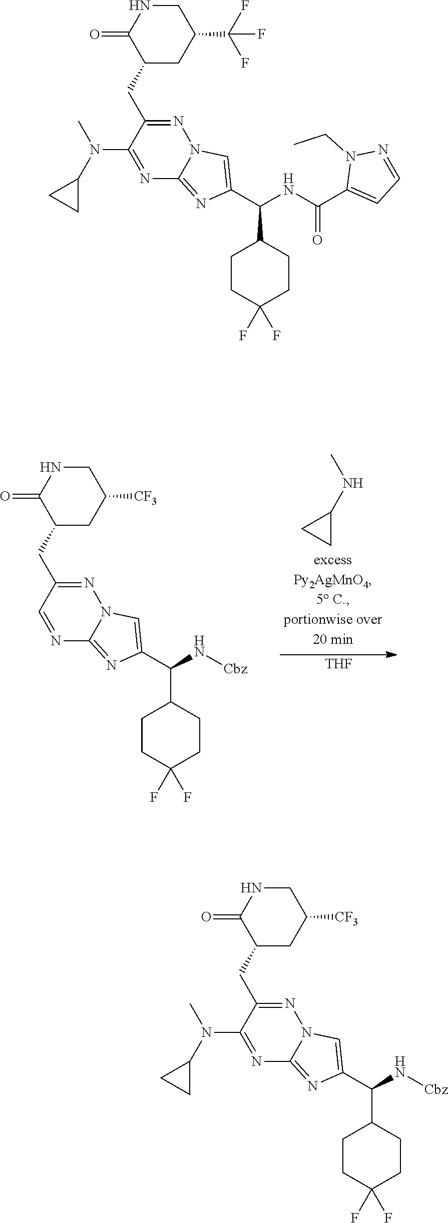

Oxidative nucleophilic substitution of hydrogen of benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (1.00 eq) with N-methylcyclopropanamine (20.0 eq) was carried out following General Procedure 8, using bis(pyridine)silver(I) permanganate (1.50 eq) in THF (0.1 M) and purifying by prep-HPLC (column: Gemini C18 250*21.2 mm*5 μm; mobile phase: [water (TFA)—acetonitrile]; gradient: 10%-55% B over 25 min) followed by a basic workup (basification with 1 M NaOH, extraction with 3× ethyl acetate, and Na₂SO₄ for drying) to afford benzyl ((S)-(3-(cyclopropyl(methyl)amino)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)carbamate as an oily yellow solid (47% yield). LCMS [M+H]⁺=650.3 m/z.

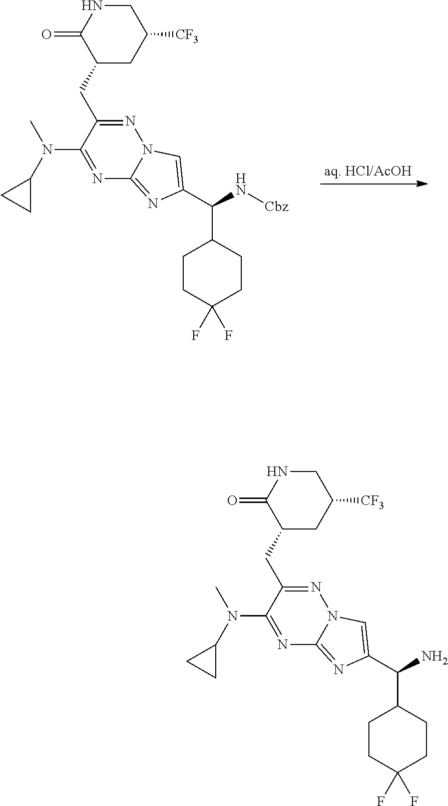 [SMILES: CN(c1nc2nc([C@@H](NC(=O)OCc3ccccc3)C3CCC(F)(F)CC3)cn2nc1C[C@H]1C[C@@H](C(F)(F)F)CNC1=O)C1CC1 ; CN(c1nc2nc([C@@H](N)C3CCC(F)(F)CC3)cn2nc1C[C@H]1C[C@@H](C(F)(F)F)CNC1=O)C1CC1]

To a solution of benzyl ((S)-(3-(cyclopropyl(methyl)amino)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)carbamate (22.4 mg, 32.7 μmol, 95% pure) in AcOH (0.65 mL, 0.05 M) was added 12 M HCl (0.27 mL, 100 eq). The reaction mixture was allowed to stir at 60° C. for 18 h. After cooling, the reaction mixture was dripped slowly into a mixture of 10 mL sat. aq. NaHCO₃ and 3 mL 1 M NaOH. This mixture was then extracted with ethyl acetate (3×), and the combined organic layers were dried over Na₂SO₄, filtered through Na₂SO₄, and concentrated to give (3R,5R)-3-((6-((S)-amino(4,4-difluorocyclohexyl)methyl)-3-(cyclopropyl(methyl)amino)imidazo[1,2-b][1,2,4]triazin-2-yl)methyl)-5-(trifluoromethyl)piperidin-2-one as a yellow oil (5.6 mg, 28% yield, 85% pure). LCMS [M+H]⁺=516.2 m/z.

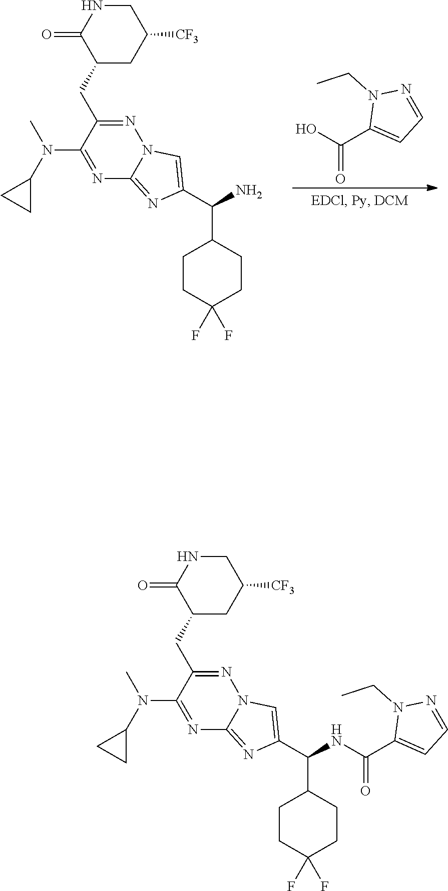

Amide coupling of (3R,5R)-3-((6-((S)-amino(4,4-difluorocyclohexyl)methyl)-3-(cyclopropyl(methyl)amino)imidazo[1,2-b][1,2,4]triazin-2-yl)methyl)-5-(trifluoromethyl)piperidin-2-one (5.6 mg, 9.2 μmol, 85% pure) with 1-ethyl-1H-pyrazole-5-carboxylic acid (2.3 mg, 16.4 μmol, 1.8 eq) using EDCI (2.4 mg, 12.5 μmol. 1.4 eq) in pyridine (0.1 mL) and dichloromethane (0.1 mL) was carried out following the conditions in General Procedure 6. The product was purified by prep-HPLC (column: Gemini C18 250*21.2 mm*5 μm; mobile phase: [water (TFA)—acetonitrile]; gradient: 10%-60% B over 25 min) followed by a basic workup (basification with 1 M NaOH, extraction with 3× ethyl acetate, and Na₂SO₄ for drying) to afford N—((S)-(3-(cyclopropyl(methyl)amino)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide, Compound 143 (2.8 mg, 4.2 μmol, 45% yield) as a yellow solid. LCMS [M+H]⁺=638.3 m/z.

### Example 66: Preparation of 1-ethyl-N—((S)-((1r,4S)-4-methylcyclohexyl)(3-((R)-2-methylmorpholino)-2-(((3R,5R)-2-oxo-5-(trifluoromethyl) Piperidin-3-yl)methyl)imidazo[1,2-b][1,2,4]triazin-6-yl)methyl)-1H-pyrazole-5-carboxamide (Compound 157)

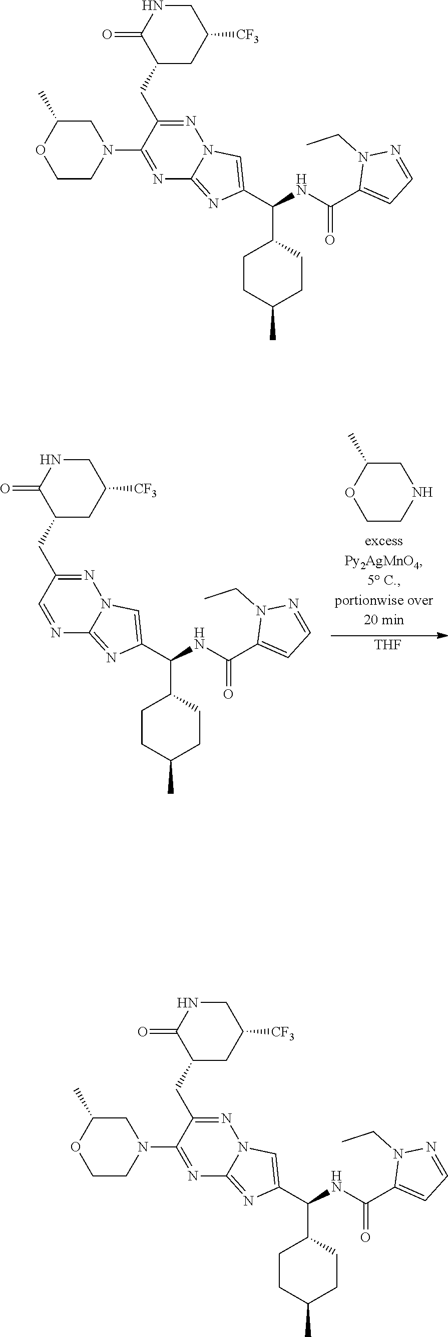

To a solution of 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (50.0 mg, 91.5 μmol, 1.00 eq) and (R)-2-methylmorpholine (185 mg, 1.83 mmol, 20.0 eq) in THF (1.00 mL) was added portion-wise bis(pyridine)silver(I) permanganate (53.1 mg, 137 μmol, 1.50 eq) at 0° C. The mixture was stirred at 5° C. for 10 min then the reaction mixture was partitioned between ethyl acetate (80.0 mL) and H₂O (80.0 mL). The organic phase was separated, washed with brine (20.0 mL*3), dried over Na₂SO₄, filtered, and concentrated under reduced pressure to give a residue. The residue was purified by prep-TLC (SiO₂, dichloromethane:MeOH=10:1). The title compound, 1-ethyl-N—((S)-((1r,4S)-4-methylcyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide, Compound 157 (22.03 mg, 32.7 μmol, 36% yield), was obtained as a yellow solid. LCMS [M+H]⁺=646.4 m/z.

### Example 67: Preparation of 1-ethyl-N—((S)-((1r,4S)-4-methylcyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (Compound 158)

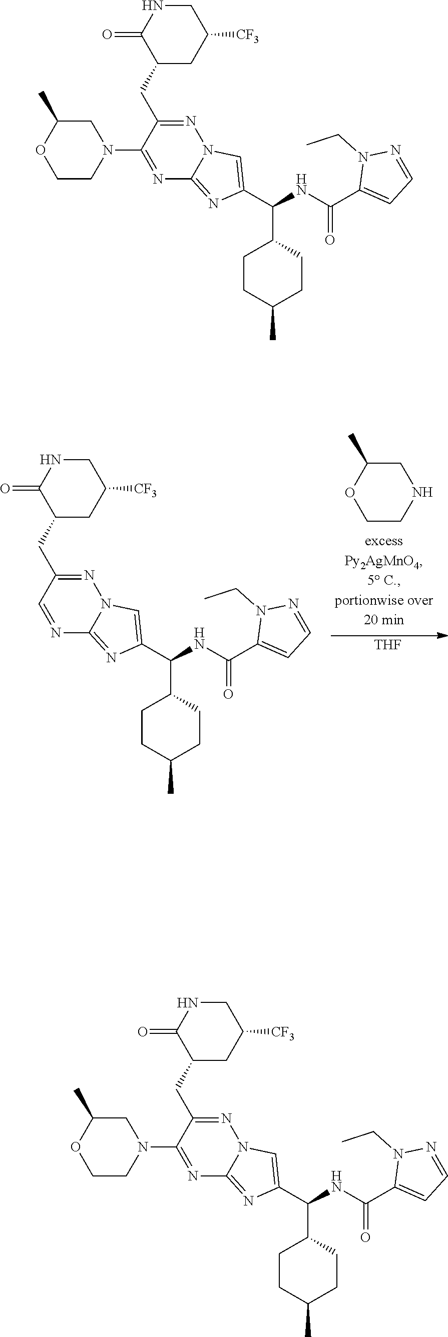

To a solution of 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (50.0 mg, 91.5 μmol, 1.00 eq) and (S)-2-methylmorpholine (185 mg, 1.83 mmol, 20.0 eq) in THF (1.00 mL) was added portion-wise bis(pyridine)silver(I) permanganate (53.1 mg, 137 μmol, 1.50 eq) at 0° C. The mixture was stirred at 5° C. for 10 min. The reaction mixture was then partitioned between ethyl acetate (80.0 mL) and H₂O (80.0 mL). The organic phase was separated, washed with brine (20.0 mL*3), dried over Na₂SO₄, filtered, and concentrated under reduced pressure to give a residue. The residue was purified by prep-TLC (SiO₂, dichloromethane:MeOH=10:1). The title compound 1-ethyl-N—((S)-((1r,4S)-4-methylcyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide Compound 158 (35.95 mg, 53.28 μmol, 58% yield), was obtained as a yellow solid. LCMS [M+H]⁺=646.4 m/z.

### Example 68: Preparation of N—((S)-(4,4-difluorocyclohexyl)(2-(((3R,5R)-2-oxo-5-(trifluoromethyl)piperidin-3-yl)methyl)-3-(8-oxa-2-azaspiro[4.5]decan-2-yl)imidazo[1,2-b][1,2,4]triazin-6-yl)methyl)-1-ethyl-1H-pyrazole-5-carboxamide (Compound 162)

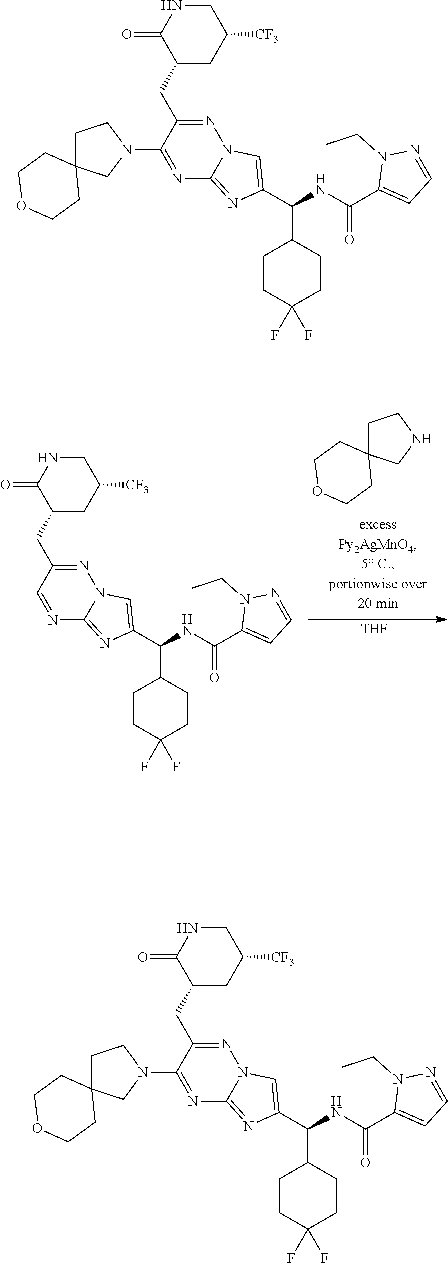 [SMILES: CCn1nccc1C(=O)N[C@H](c1cn2nc(C[C@H]3C[C@@H](C(F)(F)F)CNC3=O)c(N3CCC4(CCOCC4)C3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; C1CC2(CCOCC2)CN1 ; CCn1nccc1C(=O)N[C@H](c1cn2nc(C[C@H]3C[C@@H](C(F)(F)F)CNC3=O)c(N3CCC4(CCOCC4)C3)nc2n1)C1CCC(F)(F)CC1 ; "THF"]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with 8-oxa-2-azaspiro[4.5]decane (19.5 eq) was carried out following General Procedure 8, using bis(pyridine)silver(I) permanganate (7.70 eq) in THF (0.08 M) and purifying by prep-HPLC (column: Gemini C18 250*21.2 mm*5 μm; mobile phase: [water (TFA)—acetonitrile]; gradient: 10%-60% B over 26 min) to afford N—((S)-(4,4-difluorocyclohexyl)(2-(((3R,5R)-2-oxo-5-(trifluoromethyl)piperidin-3-yl)methyl)-3-(8-oxa-2-azaspiro[4.5]decan-2-yl)imidazo[1,2-b][1,2,4]triazin-6-yl)methyl)-1-ethyl-1H-pyrazole-5-carboxamide, Compound 162 (20% yield). LCMS [M+H]⁺=708.3 m/z.

### Example 69: Preparation of N—((S)-(4,4-difluorocyclohexyl)(3-(methyl(oxetan-3-yl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 180)

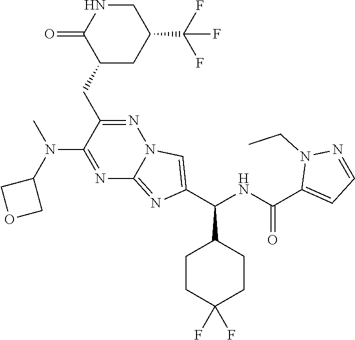 [SMILES: CCn1nccc1C(=O)N[C@H](c1cn2nc(C[C@H]3C[C@@H](C(F)(F)F)CNC3=O)c(N(C)C3COC3)nc2n1)C1CCC(F)(F)CC1]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with N-methyloxetan-3-amine (20.0 eq) was carried out following General Procedure 8, using bis(pyridine)silver(I) permanganate (5.2 eq) in THF (0.08 M) and purifying by prep-HPLC (column: Gemini C18 250*21.2 mm*5 μm; mobile phase: [water (TFA)—acetonitrile], gradient: 10%-55% B over 26 min) and lyophilizing directly to afford N—((S)-(4,4-difluorocyclohexyl)(3-(methyl(oxetan-3-yl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 180 (22% yield, trifluoroacetate salt). LCMS [M+H]⁺=654.2 m/z.

### Example 70: Preparation of (5-(trifluoromethyl)isoxazol-3-yl)methyl ((S)-(4,4-difluorocyclohexyl)(3-morpholino-2-(((3R,5R)-2-oxo-5-(trifluoromethyl)piperidin-3-yl)methyl)imidazo[1,2-b][1,2,4]triazin-6-yl)methyl)carbamate (Compound 181)

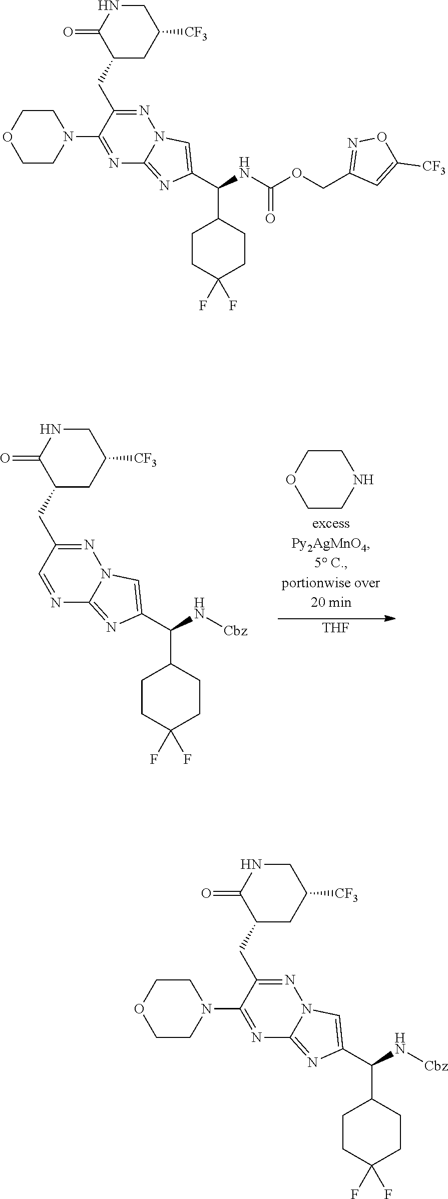 [SMILES: O=C(N[C@H](c1cn2nc(C[C@H]3C[C@@H](C(F)(F)F)CNC3=O)c(N3CCOCC3)nc2n1)C1CCC(F)(F)CC1)OCc1cc(C(F)(F)F)on1 ; O=C(N[C@H](c1cn2nc(C[C@H]3C[C@@H](C(F)(F)F)CNC3=O)cnc2n1)C1CCC(F)(F)CC1)OCc1ccccc1 ; C1COCCN1 ; O=C(N[C@H](c1cn2nc(C[C@H]3C[C@@H](C(F)(F)F)CNC3=O)c(N3CCOCC3)nc2n1)C1CCC(F)(F)CC1)OCc1ccccc1 ; "THF"]

Oxidative nucleophilic substitution of hydrogen of benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (1.00 eq) with morpholine (20.0 eq) was carried out following General Procedure 8, using bis(pyridine)silver(I) permanganate (5.00 eq) in THF (0.05 M) and purifying by column chromatography (SiO₂, dichloromethane/MeOH 1:0 to 10:1) to afford benzyl ((S)-(4,4-difluorocyclohexyl)(3-morpholino-2-(((3R,5R)-2-oxo-5-(trifluoromethyl)piperidin-3-yl)methyl)imidazo[1,2-b][1,2,4]triazin-6-yl)methyl)carbamate (100% yield). LCMS [M+H]⁺=666.3 m/z.

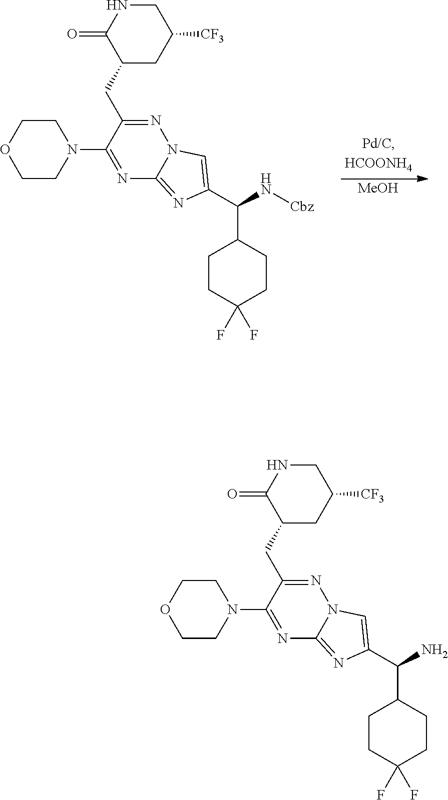 [SMILES: "MeOH" ; O=C(N[C@H](c1cn2nc(C[C@H]3C[C@@H](C(F)(F)F)CNC3=O)c(N3CCOCC3)nc2n1)C1CCC(F)(F)CC1)OCc1ccccc1 ; N[C@H](c1cn2nc(C[C@H]3C[C@@H](C(F)(F)F)CNC3=O)c(N3CCOCC3)nc2n1)C1CCC(F)(F)CC1]

To a solution of benzyl ((S)-(4,4-difluorocyclohexyl)(3-morpholino-2-(((3R,5R)-2-oxo-5-(trifluoromethyl)piperidin-3-yl)methyl)imidazo[1,2-b][1,2,4]triazin-6-yl)methyl)carbamate (35.4 mg, 52.6 μmol, 1.00 eq) in MeOH (1.05 mL) was added Pd/C (10.0% purity, 25.4 mg) and ammonium formate (65.2 mg, 1.034 mmol, 19.7 eq). The mixture was heated to 60° C. for 0.5 h, then re-charged with Pd/C (10.0% purity, 26.3 mg) and ammonium formate (87.6 mg, 1.389 mmol, 26.4 eq) and heated again to 60° C. for 18 h. After cooling, the reaction mixture was diluted with ethyl acetate, filtered through Na₂SO₄/Celite, and concentrated to give crude product, which was purified by prep-HPLC (column: Gemini C18 250*21.2 mm*5 μm; mobile phase: [water (TFA)—acetonitrile]; gradient: 10%-40% B over 25 min). Product-containing fractions were combined, concentrated to remove MeCN, basified with 1 M NaOH, and extracted with ethyl acetate (3×). The combined organic layers were dried over Na₂SO₄, filtered through Na₂SO₄, and concentrated. The product, (3R,5R)-3-((6-((S)-amino(4,4-difluorocyclohexyl)methyl)-3-morpholinoimidazo[1,2-b][1,2,4]triazin-2-yl)methyl)-5-(trifluoromethyl)piperidin-2-one (23.5 mg, 43.5 μmol, 83% yield), was obtained as a clear light-green film. LCMS [M+H]⁺=532.2 m/z.

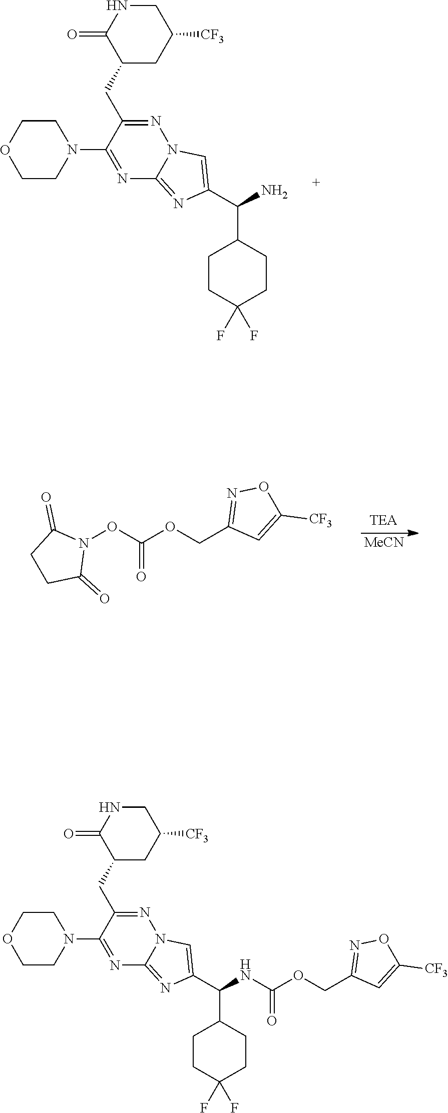

To a solution of (3R,5R)-3-((6-((S)-amino(4,4-difluorocyclohexyl)methyl)-3-morpholinoimidazo[1,2-b][1,2,4]triazin-2-yl)methyl)-5-(trifluoromethyl)piperidin-2-one (23.5 mg, 43.5 μmol, 1.00 eq) and 2,5-dioxopyrrolidin-1-yl ((5-(trifluoromethyl)isoxazol-3-yl)methyl) carbonate (20.1, 65.2 μmol, 1.50 eq) in MeCN (2.00 mL) was added TEA (18.2 uL, 130.4 μmol, 3.00 eq). The reaction mixture was stirred at room temperature for 75 min, then directly purified by prep-HPLC (column: Gemini C18 250*21.2 mm*5 μm; mobile phase: [water (TFA)—acetonitrile]; gradient: 10%-60% B over 25 min). Product-containing fractions were combined, concentrated to remove MeCN, basified with sat. aq. NaHCO₃, and extracted with ethyl acetate (3×). The combined organic layers were dried over Na₂SO₄, filtered through Na₂SO₄, and concentrated. After lyophilization, the title compound, (5-(trifluoromethyl)isoxazol-3-yl)methyl ((S)-(4,4-difluorocyclohexyl)(3-morpholino-2-(((3R,5R)-2-oxo-5-(trifluoromethyl)piperidin-3-yl)methyl)imidazo[1,2-b][1,2,4]triazin-6-yl)methyl)carbamate, Compound 181 (21.0 mg, 29.0 mol, 67% yield), was obtained as a yellow solid. LCMS [M+H]⁺=725.2 m/z.

### Example 71: Preparation of N—((S)-(4,4-difluorocyclohexyl)(3-(4,4-difluoropiperidin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 192)

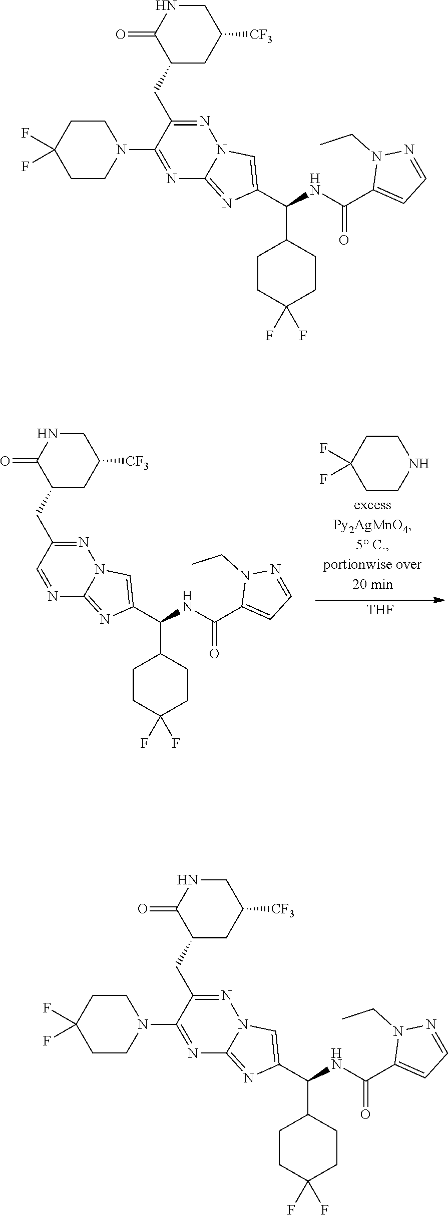 [SMILES: CCn1nccc1C(=O)N[C@H](c1cn2nc(C[C@H]3C[C@@H](C(F)(F)F)CNC3=O)c(N3CCC(F)(F)CC3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; FC1(F)CCNCC1 ; CCn1nccc1C(=O)N[C@H](c1cn2nc(C[C@H]3C[C@@H](C(F)(F)F)CNC3=O)c(N3CCC(F)(F)CC3)nc2n1)C1CCC(F)(F)CC1 ; "THF"]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with 4,4-difluoropiperidine (20.0 eq) was carried out following General Procedure 8, using bis(pyridine)silver(I) permanganate (2.46 eq) in THF (0.06 M) and purifying by prep-HPLC (column: Gemini C18 250*21.2 mm*5 μm; mobile phase: [water (TFA)—acetonitrile]; gradient: 10%-50% B over 30 min), followed by basic workup using sat. aq. NaHCO₃ and ethyl acetate, to afford N—((S)-(4,4-difluorocyclohexyl)(3-(4,4-difluoropiperidin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 192 (46% yield). LCMS [M+H]⁺=688.3 m/z.

### Example 72: Preparation of N—((S)-(4,4-difluorocyclohexyl)(2-(((3R,5R)-2-oxo-5-(trifluoromethyl)piperidin-3-yl)methyl)-3-(6-oxa-2-azaspiro[3.4]octan-2-yl)imidazo[1,2-b][1,2,4]triazin-6-yl)methyl)-1-ethyl-1H-pyrazole-5-carboxamide (Compound 193)

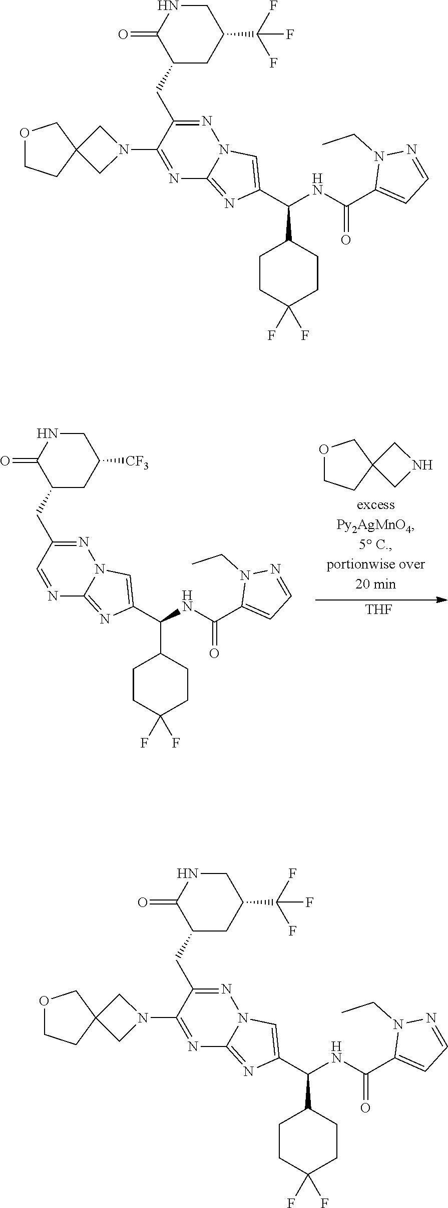 [SMILES: CCn1nccc1C(=O)N[C@H](c1cn2nc(C[C@H]3C[C@@H](C(F)(F)F)CNC3=O)c(N3CC4(CCOC4)C3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; C1CC2(CNC2)CO1 ; CCn1nccc1C(=O)N[C@H](c1cn2nc(C[C@H]3C[C@@H](C(F)(F)F)CNC3=O)c(N3CC4(CCOC4)C3)nc2n1)C1CCC(F)(F)CC1 ; "THF"]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with 6-oxa-2-azaspiro[3.4]octane (20.0 eq) was carried out following General Procedure 8, using bis(pyridine)silver(I) permanganate (1.80 eq) in THF (0.08 M) and purifying by prep-HPLC (column: Gemini C18 250*21.2 mm*5 μm; mobile phase: [water (TFA)—acetonitrile]; gradient: 10%-50% B over 25 min), followed by basic workup using sat. aq. NaHCO₃ and ethyl acetate, to afford N—((S)-(4,4-difluorocyclohexyl)(2-(((3R,5R)-2-oxo-5-(trifluoromethyl)piperidin-3-yl)methyl)-3-(6-oxa-2-azaspiro[3.4]octan-2-yl)imidazo[1,2-b][1,2,4]triazin-6-yl)methyl)-1-ethyl-1H-pyrazole-5-carboxamide, Compound 193 (75% yield). LCMS [M+H]⁺=680.3 m/z.

### Example 73: Preparation of N—((S)-(4,4-difluorocyclohexyl)(3-(4-hydroxy-4-methylpiperidin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 194)

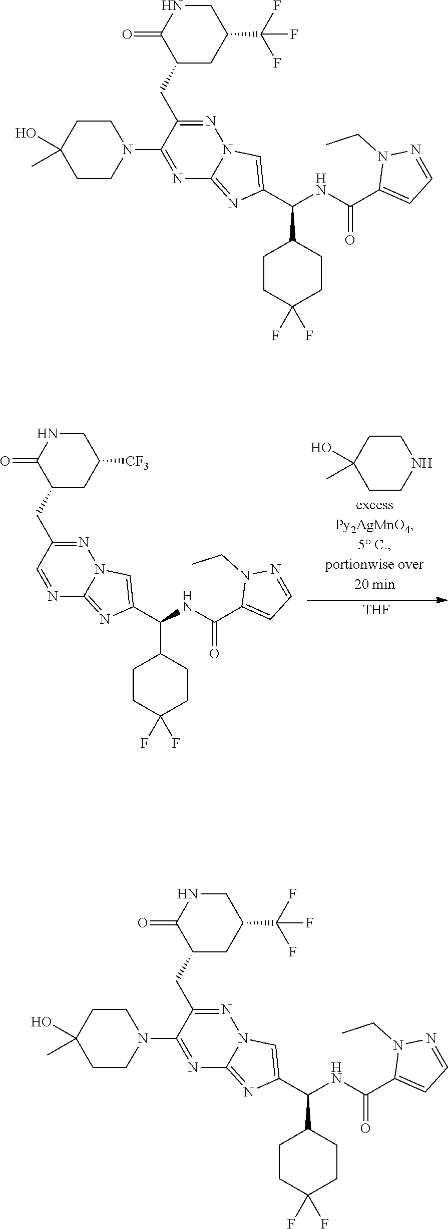

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with 4-methylpiperidin-4-ol (9.7 eq) was carried out following General Procedure 8, using bis(pyridine)silver(I) permanganate (1.63 eq) in THF (0.06 M) and purifying by prep-HPLC (column: Gemini C18 250*21.2 mm*5 μm; mobile phase: [water (TFA)—acetonitrile]; gradient: 10%-50% B over 30 min), followed by basic workup using sat. aq. NaHCO₃ and ethyl acetate, to afford N—((S)-(4,4-difluorocyclohexyl)(3-(4-hydroxy-4-methylpiperidin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide Compound 194 (68% yield). LCMS [M+H]⁺=682.3 m/z.

### Example 74: Preparation of N—((S)-(4,4-difluorocyclohexyl)(3-(methyl((3-methyloxetan-3-yl)methyl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 195)

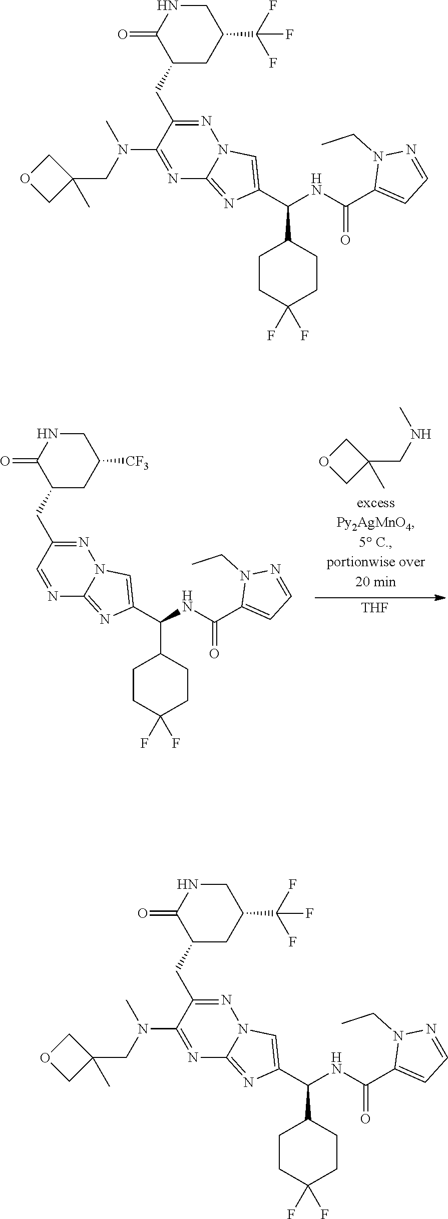

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.00 eq) with N-methyl-1-(3-methyloxetan-3-yl)methanamine (20.0 eq) was carried out following General Procedure 8, using bis(pyridine)silver(I) permanganate (4.9 eq) in THF (0.065 M) and purifying by prep-HPLC (column: Gemini C18 250*21.2 mm*5 μm; mobile phase: [water (TFA)—acetonitrile]; gradient: 10%-47% B over 40 min and directly lyophilizing to afford N—((S)-(4,4-difluorocyclohexyl)(3-(methyl((3-methyloxetan-3-yl)methyl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 195 (11% yield, ~80% purity, trifluoroacetate salt). LCMS [M+H]⁺=682.3 m/z.

### Example 75: Preparation of N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (Compound 29)

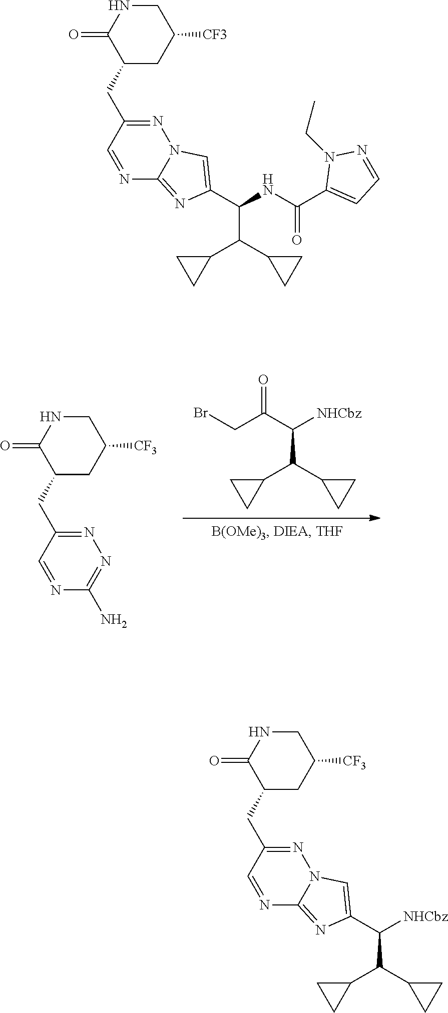

The synthesis of benzyl ((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)carbamate was carried out following General Procedure 3 using (3R,5R)-3-((3-amino-1,2,4-triazin-6-yl)methyl)-5-(trifluoromethyl)piperidin-2-one (1.00 eq) and benzyl (S)-(4-bromo-1,1-dicyclopropyl-3-oxobutan-2-yl)carbamate (1.10 eq) and was purified by prep-TLC (SiO₂, Petroleum ether:Ethyl acetate=0:1) to afford benzyl ((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)carbamate (67% yield) as a white solid. LCMS [M+H]⁺=557.3 m/z.

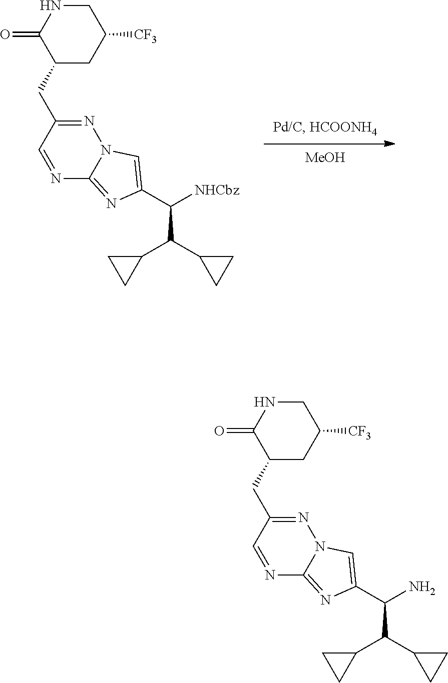 [SMILES: "MeOH" ; O=C(N[C@H](c1cn2nc(C[C@H]3C[C@@H](C(F)(F)F)CNC3=O)cnc2n1)C(C1CC1)C1CC1)OCc1ccccc1 ; "Pd/C, HCOONH4" ; N[C@H](c1cn2nc(C[C@H]3C[C@@H](C(F)(F)F)CNC3=O)cnc2n1)C(C1CC1)C1CC1]

To a solution of benzyl ((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)carbamate (120 mg, 215 μmol, 1.00 eq) in MeOH (1 mL) was added ammonium formate (40.7 mg, 646 μmol, 3.00 eq) and Pd/C (20.0 mg, 10% purity) under N₂ atmosphere. The mixture was stirred at room temperature for 1 h. TLC (dichloromethane/MeOH=10:1) indicated starting material was consumed completely and one new spot formed. The reaction mixture was filtered and the cake was washed with 100 mL MeOH, then the filtrate was concentrated under reduced pressure to give a residue. To the residue was added sat. aq. NaHCO₃ to adjust pH to 9, and the resulting mixture was extracted with ethyl acetate (30.0 mL*3). The combined organic layers were washed with sat. aq. NaHCO₃ (30.0 mL*2), dried over Na₂SO₄, filtered, and concentrated under reduced pressure to afford (3R,5R)-3-((6-((S)-1-amino-2,2-dicyclopropylethyl)imidazo[1,2-b][1,2,4]triazin-2-yl)methyl)-5-(trifluoromethyl)piperidin-2-one (90.0 mg, 213 μmol, 99% yield) as a white solid.

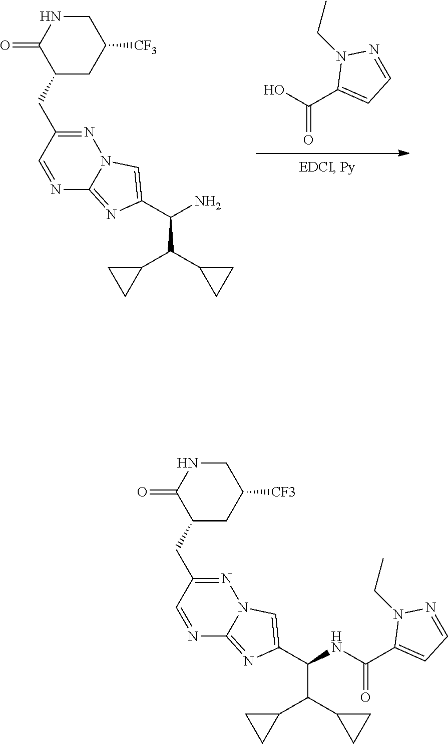

Amide coupling of (3R,5R)-3-((6-((S)-1-amino-2,2-dicyclopropylethyl)imidazo[1,2-b][1,2,4]triazin-2-yl)methyl)-5-(trifluoromethyl)piperidin-2-one (35.0 mg, 82.8 μmol, 1.00 eq) with 1-ethyl-1H-pyrazole-5-carboxylic acid (13.9 mg, 99.4 μmol, 1.20 eq) using EDCI (47.6 mg, 248 μmol, 3.00 eq) in pyridine (3.00 mL) was carried out following the conditions in General Procedure 6. The product was purified by prep-TLC (SiO₂, dichloromethane/MeOH=100:1 to 10:1) to afford N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide, Compound 29 (30.3 mg, 54.9 μmol, 66% yield) as a yellow solid. LCMS [M+H]⁺=545.3 m/z.

### Example 76: Preparation of 4-cyclopropyl-N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1,2,5-oxadiazole-3-carboxamide (Compound 39)

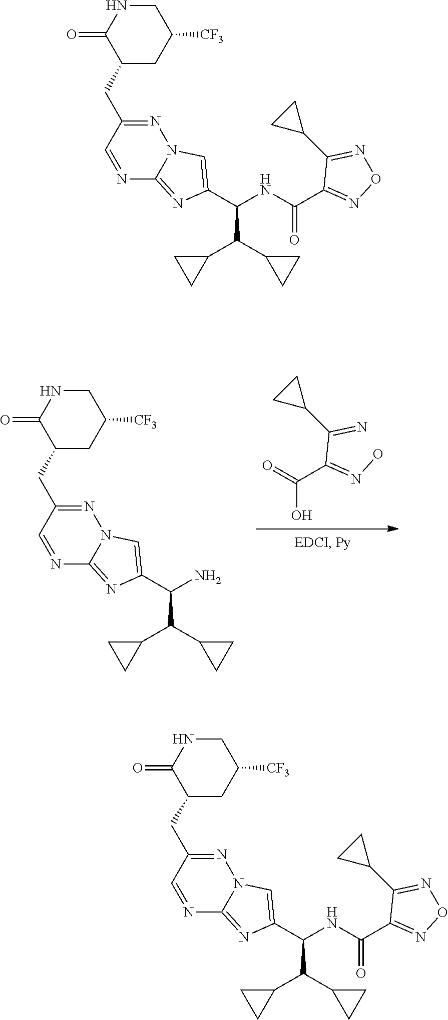

Amide coupling of (3R,5R)-3-((6-((S)-1-amino-2,2-dicyclopropylethyl)imidazo[1,2-b][1,2,4]triazin-2-yl)methyl)-5-(trifluoromethyl)piperidin-2-one (40.0 mg, 94.6 μmol, 1.00 eq) with 4-cyclopropyl-1,2,5-oxadiazole-3-carboxylic acid (17.5 mg, 113 μmol, 1.20 eq) using EDCI (54.4 mg, 284 μmol, 3.00 eq) in pyridine (1.00 mL) was carried out following the conditions in General Procedure 6. The product was purified by prep-TLC (SiO₂, dichloromethane/MeOH=10:1) to afford 4-cyclopropyl-N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1,2,5-oxadiazole-3-carboxamide, Compound 39 (30.63 mg, 53.5 μmol, 57% yield) as a white solid. LCMS [M+H]⁺=559.2 m/z.

### Example 77: Preparation of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 30)

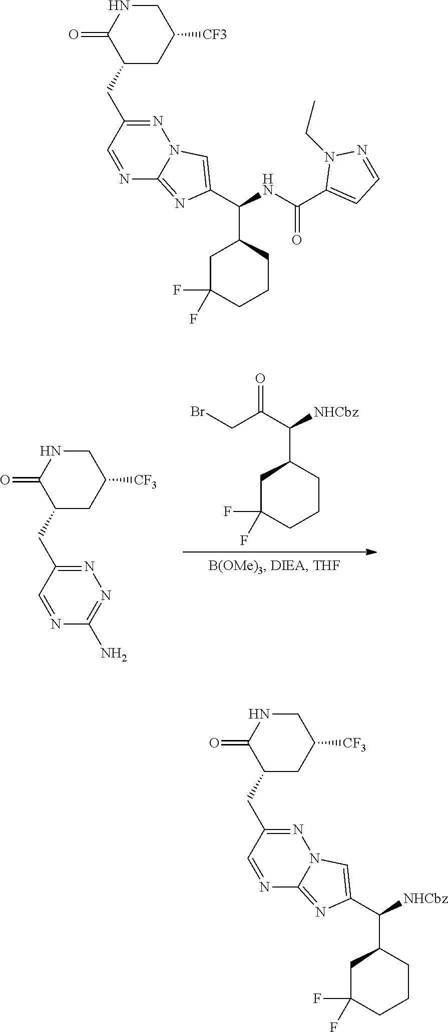

The synthesis of benzyl ((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate was carried out following General Procedure 3 using (3R,5R)-3-((3-amino-1,2,4-triazin-6-yl)methyl)-5-(trifluoromethyl)piperidin-2-one (1.00 eq) and benzyl ((S)-3-bromo-1-((S)-3,3-difluorocyclohexyl)-2-oxopropyl)carbamate (1.40 eq) and was purified by prep-TLC (SiO₂, Petroleum ether:Ethyl acetate=0:1 to 1:0) to afford benzyl ((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (47% yield) as a yellow oil. LCMS [M+H]⁺=581.2 m z

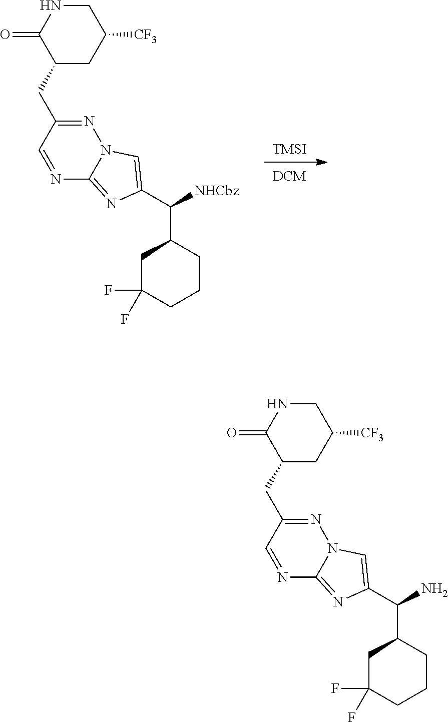

The synthesis of (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one was carried out following General Procedure 4 using benzyl ((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (1.00 eq) and TMSI (3.00 eq) in dichloromethane (0.16 M) to afford crude (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one as a yellow oil. LCMS [M+H]⁺=447.2 m/z.

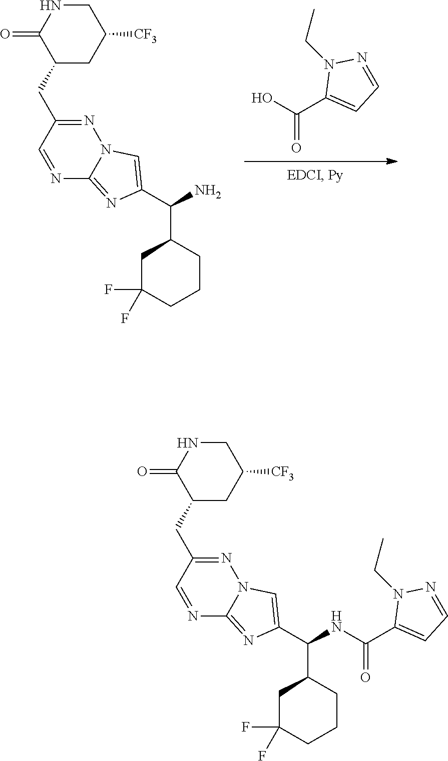

Amide coupling of (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one (45.0 mg, 101 μmol, 1.00 eq) with 1-ethyl-1H-pyrazole-5-carboxylic acid (17.0 mg, 121 μmol, 1.20 eq) using EDCI (34.8 mg, 181 μmol, 1.80 eq) in pyridine (1.00 mL) was carried out following the conditions in General Procedure 6. The product was purified by prep-HPLC (FA condition, column: Phenomenex luna C 18 150*25 mm*10 μm; mobile phase: [water (FA)—acetonitrile]; gradient: 35%-65% B over 10 min) to afford N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 30 (13.1 mg, 22.8 μmol, 23% yield) as a yellow solid. LCMS [M+H]⁺=569.3 m/z.

### Example 78: Preparation of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 31)

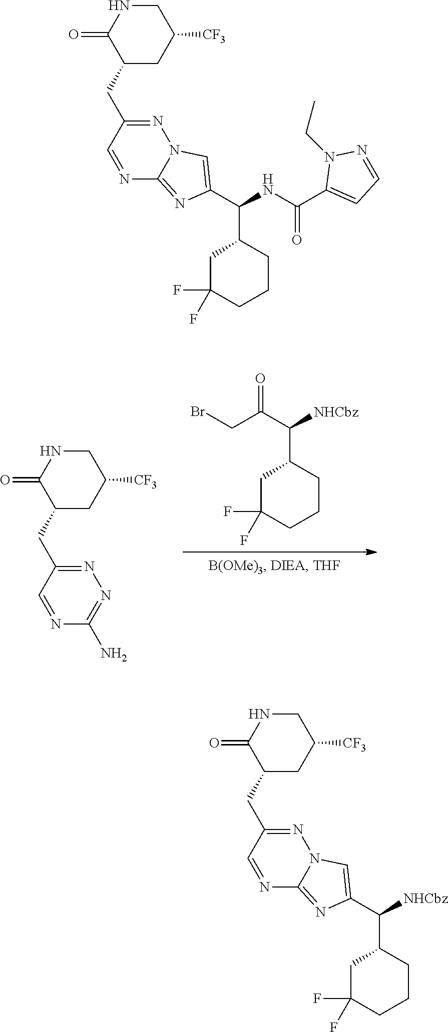

The synthesis of benzyl ((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate was carried out following General Procedure 3 using (3R,5R)-3-((3-amino-1,2,4-triazin-6-yl)methyl)-5-(trifluoromethyl)piperidin-2-one (1.00 eq) and benzyl ((S)-3-bromo-1-((R)-3,3-difluorocyclohexyl)-2-oxopropyl)carbamate (1.40 eq) and was purified by prep-TLC (SiO₂, Petroleum ether:Ethyl acetate=0:1) to afford benzyl ((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (52% yield) as a yellow oil. LCMS [M+H]⁺=581.4 m/z.

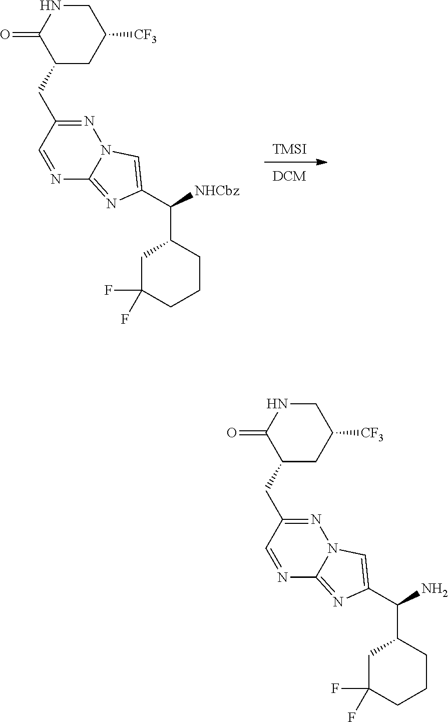

The synthesis of (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one was carried out following General Procedure 4 using benzyl ((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (1.00 eq) and TMSI (3.00 eq) in dichloromethane (0.17 M) to afford crude (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one as a yellow oil. LCMS [M+H]⁺=447.2 m/z.

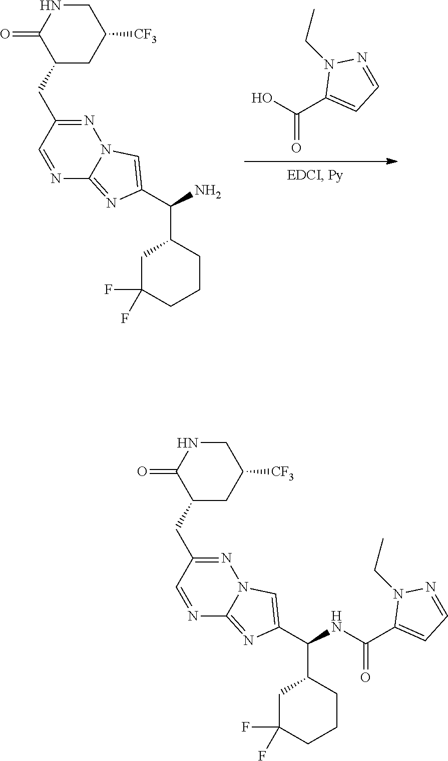

Amide coupling of (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one (60.0 mg, 134 μmol, 1.00 eq) with 1-ethyl-1H-pyrazole-5-carboxylic acid (22.6 mg, 161 μmol, 1.20 eq) using EDCI (46.4 mg, 242 μmol, 1.80 eq) in pyridine (3.00 mL) was carried out following the conditions in General Procedure 6. The product was purified by prep-HPLC (FA condition, column: Phenomenex luna C 18 150*25 mm*10 μm; mobile phase: [water (FA)—acetonitrile]; gradient: 36%-66% B over 10 min) then SFC (column: DAICEL CHIRALPAK IG (250 mm*30 mm, 10 μm); mobile phase: [CO₂-iPrOH (0.1% NH₃H₂O)]; B %: 40%, isocratic elution mode) to afford N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 31 (25.3 mg, 44.4 μmol, 33% yield) as the first eluting single stereoisomer, obtained as a yellow solid. LCMS [M+H]⁺=569.3 m/z.

### Example 79: Preparation of 4-cyclopropyl-N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 44)

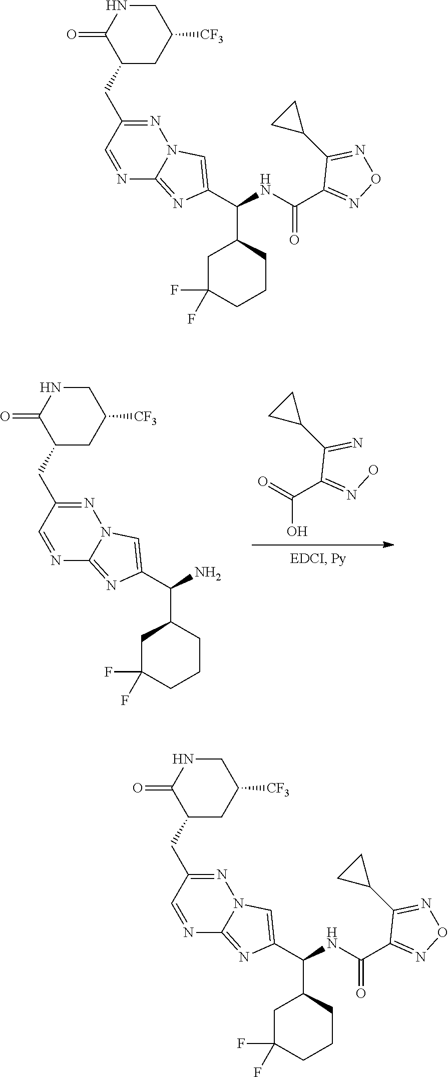

Amide coupling of (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one with 4-cyclopropyl-1,2,5-oxadiazole-3-carboxylic acid using EDCI in pyridine was carried out following the conditions in General Procedure 6, in the same manner as the synthesis of Compound 30 with prep-TLC purification to afford 4-cyclopropyl-N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide, Compound 44 (19% yield), obtained as a yellow solid. LCMS [M+H]⁺=583.3 m/z.

### Example 80: Preparation of 4-cyclopropyl-N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 45)

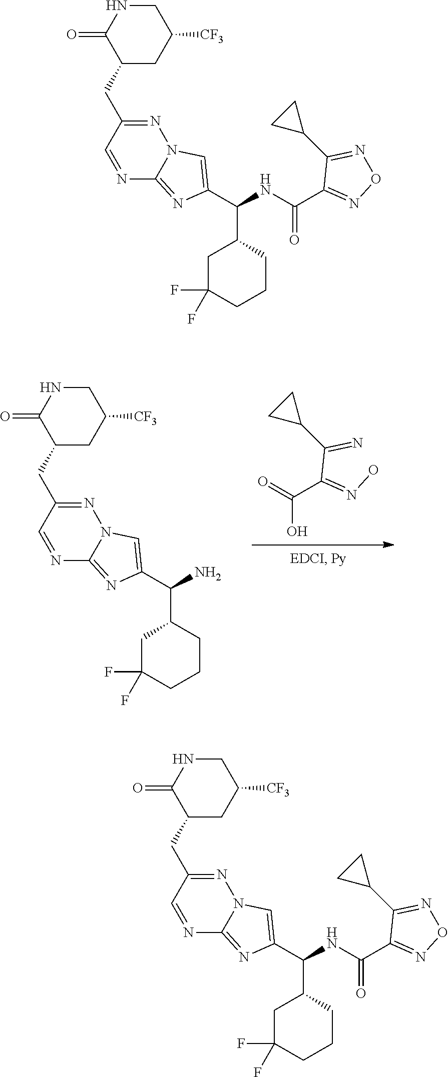

Amide coupling of (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one with 4-cyclopropyl-1,2,5-oxadiazole-3-carboxylic acid using EDCI in pyridine was carried out following the conditions in General Procedure 6, in the same manner as the synthesis of 22094 with prep-TLC purification (dichloromethane:MeOH=10:1, Rf=0.5) and SFC purification (column: DAICEL CHIRALPAK AD (250 mm*30 mm, 10 μm); mobile phase: [CO₂-MeOH (0.1% NH₃H₂O)]; B %: 45%, isocratic elution mode) to afford 4-cyclopropyl-N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide, Compound 45 (19% yield), obtained as a white solid. LCMS [M+H]⁺=583.4 m/z.

### Example 81: Preparation of 4-cyclopropyl-N-((1S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 33)

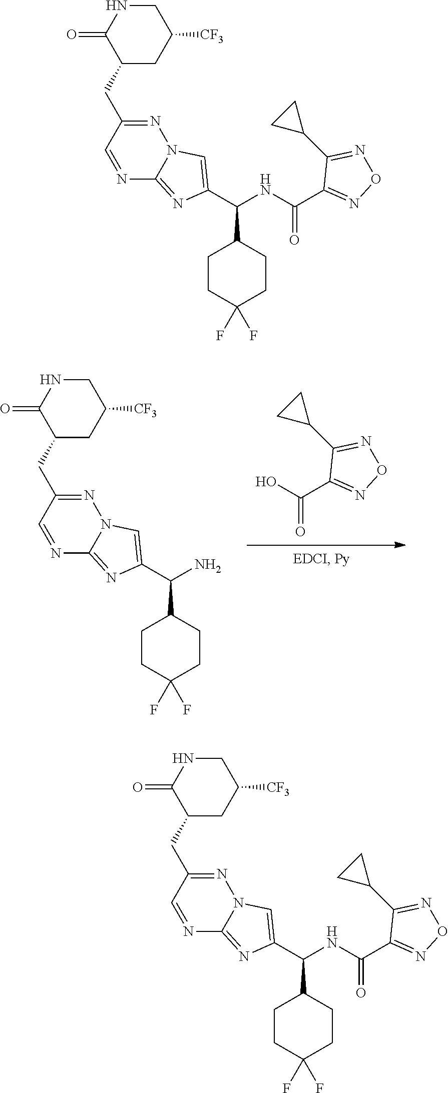

Amide coupling of (5R)-3-((6-((S)-amino(4,4-difluorocyclohexyl)methyl)imidazo[1,2-b][1,2,4]triazin-2-yl)methyl)-5-(trifluoromethyl)piperidin-2-one (200 mg, 448 μmol, 1.00 eq) with 4-cyclopropyl-1,2,5-oxadiazole-3-carboxylic acid (75.9 mg, 492 μmol, 1.10 eq) using EDCI (257 mg, 1.34 mmol, 3.00 eq) in pyridine (2.00) was carried out following the conditions in General Procedure 6. The product was purified by prep-TLC (SiO₂, dichloromethane/MeOH=10:1) to afford the mixture of stereoisomers (70.0 mg, 117 μmol, 26% yield) as a white solid, which was further purified by SFC (condition: column: DAICEL CHIRALPAK IC (250 mm*30 mm, 10 μm); mobile phase: [CO₂-i-PrOH (0.1% NH₃H₂O)], B %: 35%-35%, 4.2 min). The first eluting single stereoisomer of 4-cyclopropyl-N-((1S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)-1,2,5-oxadiazole-3-carboxamide, Compound 33 (20.65 mg, 33.32 μmol, 28% yield) was obtained as a yellow solid. LCMS [M+H]⁺=583.2 m/z.

### Example 82: Preparation of 1-ethyl-N-((1S,2S)-2-(4-fluorophenyl)-1-(2-(((3R,5R)-2-oxo-5-(trifluoromethyl)piperidin-3-yl)methyl)imidazo[1,2-b][1,2,4]triazin-6-yl)butyl)-1H-pyrazole-5-carboxamide (Compound 60)

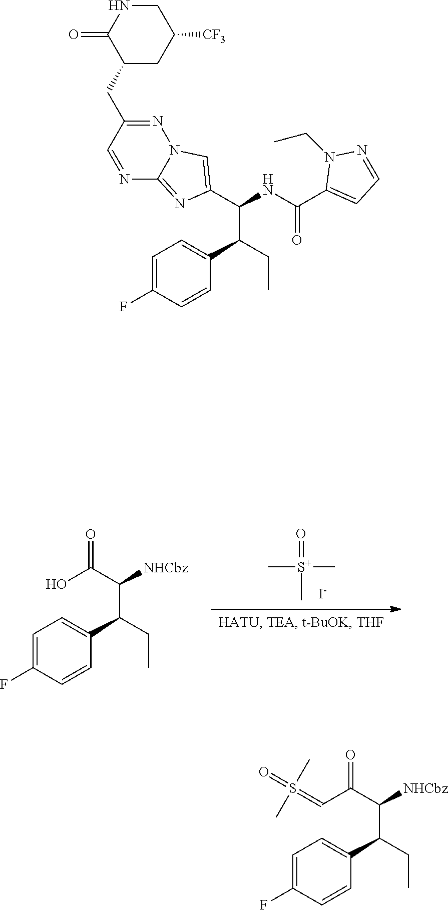

The preparation of mixture A: Trimethylsulfoxonium iodide (9.56 g, 43.43 mmol, 3.00 eq) was added to a solution of t-BuOK (4.87 g, 43.4 mmol, 3.00 eq) in THF (100 mL) and the resulting mixture was stirred at 65° C. for 1.5 h under N₂. After that time, the solution was cooled to 0° C. to obtain the mixture A. The preparation of mixture B: To a flask was added (2S,3S)-2-(((benzyloxy)carbonyl)amino)-3-(4-fluorophenyl)pentanoic acid (5.00 g, 14.5 mmol, 1.00 eq) and THF (150 mL), then TEA (2.20 g, 21.7 mmol, 3.02 mL, 1.50 eq) and HATU (7.16 g, 18.8 mmol, 1.30 eq) were added at 0° C. The resulting mixture was stirred at room temperature for 1.5 h. The suspension was cooled to 0° C. to obtain the mixture B. Mixture B was then added to mixture A at 0° C. The resulting reaction mixture was stirred at room temperature for 1.5 h, then diluted with H₂O (200 mL) and extracted with ethyl acetate (200 mL*2). The combined organic layers were washed with H₂O (300 mL) and brine (200 mL), dried over Na₂SO₄, filtered, and concentrated under reduced pressure to give a residue. The residue was purified by column chromatography (SiO₂, petroleum ether:ethyl acetate=1:1 to 6:7) to give benzyl ((3S,4S)-1-(dimethyl(oxo)-λ⁶-sulfaneylidene)-4-(4-fluorophenyl)-2-oxohexan-3-yl)carbamate (2.80 g, 6.67 mmol, 46% yield) as a yellow gum. LCMS [M+H]⁺=420.2 m/z.

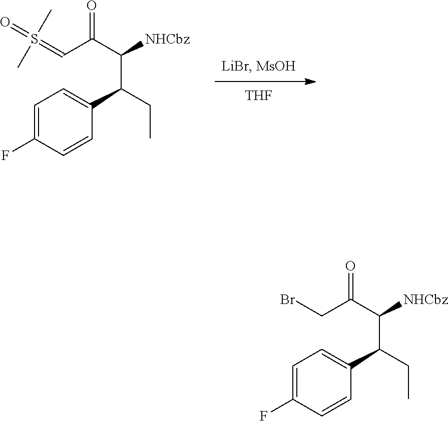

To a solution of benzyl ((3S,4S)-1-(dimethyl(oxo)-λ⁶-sulfaneylidene)-4-(4-fluorophenyl)-2-oxohexan-3-yl)carbamate (300 mg, 715 μmol, 1.00 eq) in THF (2.00 mL) was added MsOH (75.6 mg, 786 μmol, 56.2 μL, 1.10 eq) and LiBr (68.3 mg, 786 μmol, 19.8 μL, 1.10 eq). The mixture was stirred at 45° C. for 4 h. Then, the reaction mixture was diluted with H₂O (40.0 mL) and extracted with ethyl acetate (40.0 mL*3). The combined organic layers were dried over Na₂SO₄, filtered, and concentrated under reduced pressure to give a residue. The product, benzyl ((3S,4S)-1-bromo-4-(4-fluorophenyl)-2-oxohexan-3-yl)carbamate (235 mg, 556 μmol, 78% yield) was obtained as a yellow oil. LCMS [M+H]⁺=422.1 m/z.

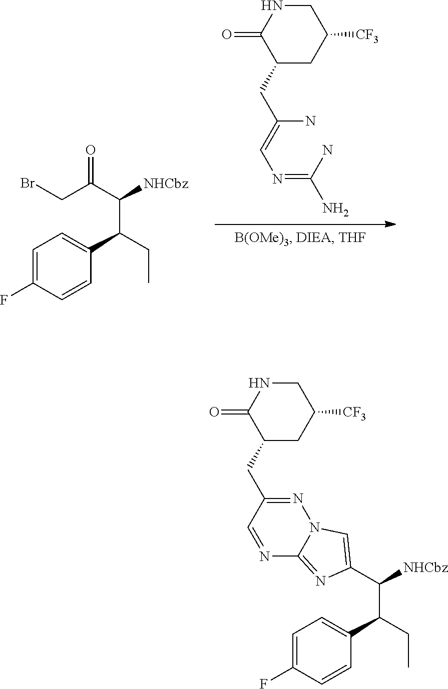

To a solution of benzyl ((3S,4S)-1-bromo-4-(4-fluorophenyl)-2-oxohexan-3-yl)carbamate (235 mg, 556 μmol, 1.00 eq) and (3R,5R)-3-((3-amino-1,2,4-triazin-6-yl)methyl)-5-(trifluoromethyl)piperidin-2-one (76.6 mg, 278 μmol, 0.500 eq) in THF (2.00 mL) was added B(OMe)₃ (289 mg, 2.78 mmol, 314 μL, 5.00 eq) and DIEA (359 mg, 2.78 mmol, 484 μL, 5.00 eq). The mixture was stirred at 70° C. for 2 h. The reaction mixture was cooled to room temperature, diluted with H₂O (40.0 mL), and extracted with ethyl acetate (40.0 mL*3). The combined organic layers were dried over Na₂SO₄, filtered, and concentrated under reduced pressure to give a residue. The residue was purified by prep-TLC (SiO₂, Dichloromethane:Methanol=10:1 to give benzyl ((1S,2S)-2-(4-fluorophenyl)-1-(2-(((3R,5R)-2-oxo-5-(trifluoromethyl)piperidin-3-yl)methyl)imidazo[1,2-b][1,2,4]triazin-6-yl)butyl)carbamate (95.0 mg, 158 μmol, 29% yield) as a yellow solid.

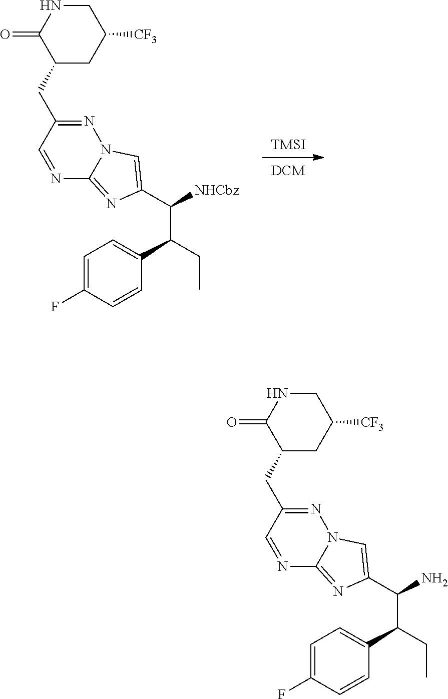

The synthesis of (3R,5R)-3-((6-((1S,2S)-1-amino-2-(4-fluorophenyl)butyl)imidazo[1,2-b][1,2,4]triazin-2-yl)methyl)-5-(trifluoromethyl)piperidin-2-one was carried out following General Procedure 4 using benzyl ((1S,2S)-2-(4-fluorophenyl)-1-(2-(((3R,5R)-2-oxo-5-(trifluoromethyl)piperidin-3-yl)methyl)imidazo[1,2-b][1,2,4]triazin-6-yl)butyl)carbamate (1.00 eq) and TMSI (3.00 eq) in dichloromethane (0.17 M) to afford (3R,5R)-3-((6-((1S,2S)-1-amino-2-(4-fluorophenyl)butyl)imidazo[1,2-b][1,2,4]triazin-2-yl)methyl)-5-(trifluoromethyl)piperidin-2-one (50% yield) as a yellow oil. LCMS [M+H]⁺=465.2 m/z.

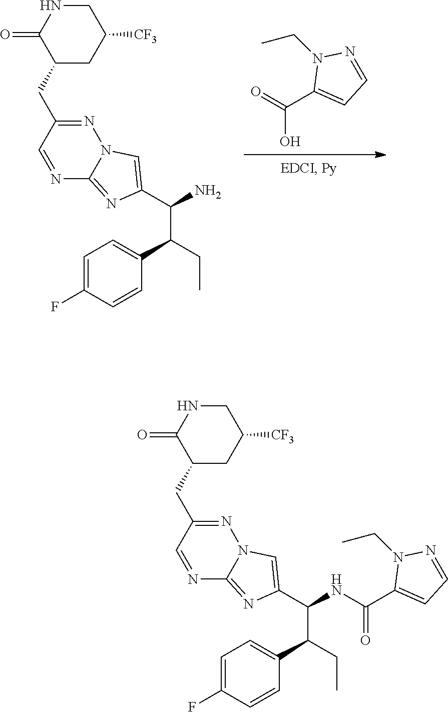

Amide coupling of (3R,5R)-3-((6-((1S,2S)-1-amino-2-(4-fluorophenyl)butyl)imidazo[1,2-b][1,2,4]triazin-2-yl)methyl)-5-(trifluoromethyl)piperidin-2-one (35.0 mg, 75.4 μmol, 1.00 eq) with 1-ethyl-1H-pyrazole-5-carboxylic acid (11.6 mg, 82.9 μmol, 1.10 eq) using EDCI (28.9 mg, 151 μmol, 2.00 eq) in pyridine (0.500 mL) was carried out following the conditions in General Procedure 6. The product was purified by prep-TLC (dichloromethane:MeOH=10:1) to afford 1-ethyl-N-((1S,2S)-2-(4-fluorophenyl)-1-(2-(((3R,5R)-2-oxo-5-(trifluoromethyl)piperidin-3-yl)methyl)imidazo[1,2-b][1,2,4]triazin-6-yl)butyl)-1H-pyrazole-5-carboxamide, Compound 60 (8.70 mg, 13.9 μmol, 18% yield) as a white solid. LCMS [M+H]⁺=587.4 m/z.

### Example 83: Preparation of N-((1S)-(2-((5,5-difluoro-2-oxopiperidin-3-yl)methyl)imidazo[1,2-b][1,2,4]triazin-6-yl)(4,4-difluorocyclohexyl)methyl)-1-ethyl-1H-pyrazole-5-carboxamide (Compound 63)

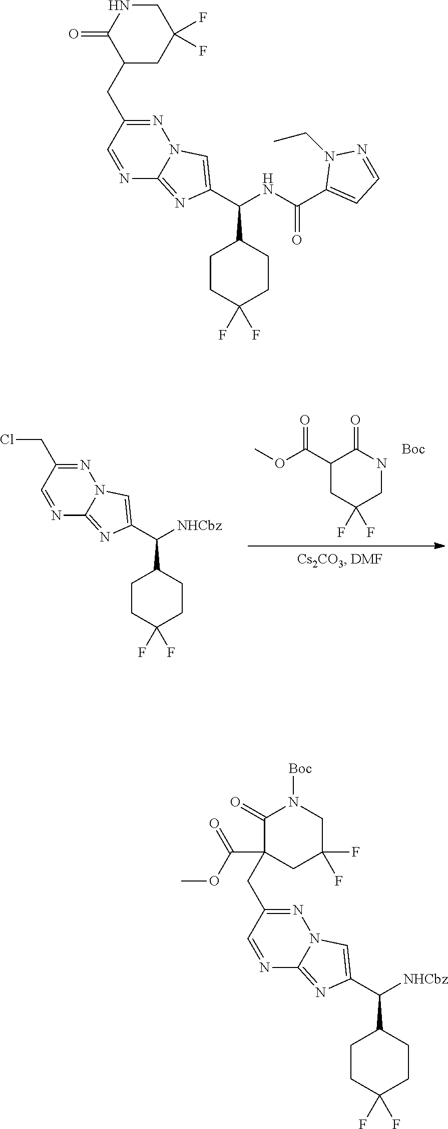

To a solution of benzyl (S)-((2-(chloromethyl)imidazo[1,2-b][1,2,4]triazin-6-yl)(4,4-difluorocyclohexyl)methyl)carbamate (847 mg, 2.89 mmol, 1.30 eq) in N,N-dimethylformamide (10.0 mL) was added Cs₂CO₃ (2.17 g, 6.67 mmol, 3.00 eq) and the mixture was stirred at room temperature for 1 h. To the mixture was added 1-(tert-butyl) 3-methyl 5,5-difluoro-2-oxopiperidine-1,3-dicarboxylate (1.00 g, 2.22 mmol, 1.00 eq) followed by stirring at 25° C. for 2 h. The reaction mixture was diluted with H₂O (40.0 mL) and extracted with dichloromethane (40.0 mL*3). The combined organic layers were washed with brine (40.0 mL*3), dried over Na₂SO₄, filtered, and concentrated under reduced pressure to give a residue that was purified by column chromatography (SiO₂, petroleum ether:ethyl acetate=1:0 to 0:1) to afford 1-(tert-butyl) 3-methyl 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-1,3-dicarboxylate (1.00 g, crude) as a white solid. LCMS [M+H]⁺=707.3 m/z.

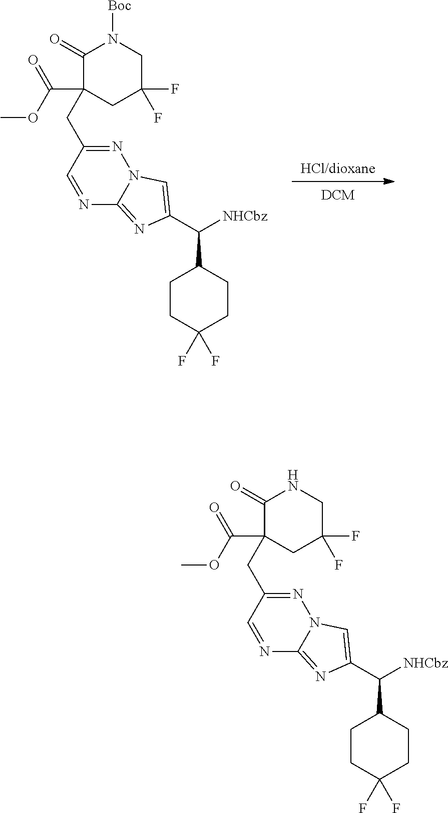

To a solution of 1-(tert-butyl) 3-methyl 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-1,3-dicarboxylate (0.950 g, 1.34 mmol, 1.00 eq) in dichloromethane (3.00 mL) was added HCl/dioxane (4.00 M, 3.00 mL, 8.93 eq). The mixture was stirred at room temperature for 0.5 h. The reaction mixture was concentrated under reduced pressure to give methyl 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-3-carboxylate (815 mg, crude) as a white solid. LCMS [M+H]⁺=607.2 m/z.

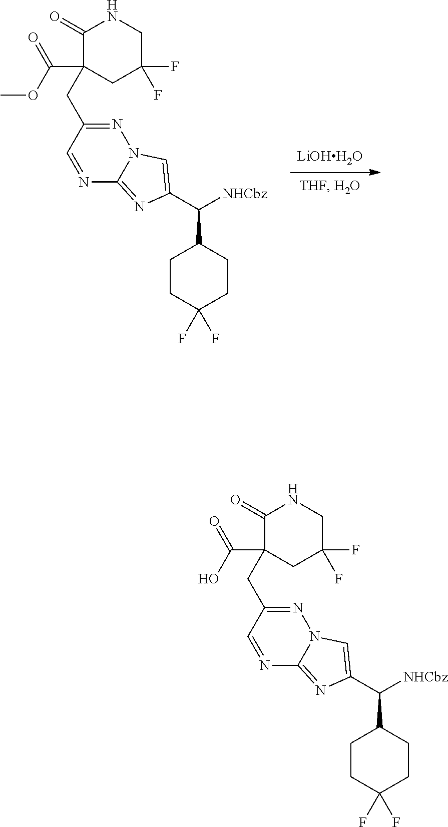

To a solution of methyl 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-3-carboxylate (815 mg, 1.34 mmol, 1.00 eq) in THF (6.00 mL) was added LiOH·H₂O (169 mg, 4.03 mmol, 3.00 eq) in H₂O (1.00 mL). The mixture was stirred at 0° C. for 1 h. The reaction mixture was washed with ethyl acetate (20.0 mL*2). The pH of the aqueous phase was adjusted to 3 with 1 M HCl and extracted with ethyl acetate (20.0 mL*2). The combined organic layers were dried over Na₂SO₄, filtered, and concentrated under reduced pressure to give crude 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-3-carboxylic acid (780 mg, crude) was obtained as a white solid. LCMS [M+H]⁺=593.2 m/z.

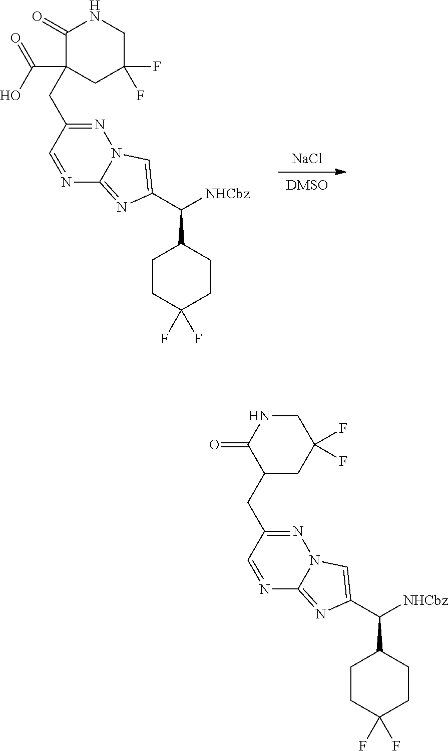

To a solution of 3-((6-((S)-(((benzyloxy)carbonyl)amino)(4,4-difluorocyclohexyl)methyl)imidazo[1,2-b][1,2,4]triazin-2-yl)methyl)-5,5-difluoro-2-oxopiperidine-3-carboxylic acid (700 mg, 1.18 mmol, 1.00 eq) in N,N-dimethylformamide (26.0 mL) was added NaCl (345 mg, 5.91 mmol, 5.00 eq). The mixture was stirred at 110° C. for 1 h. The reaction mixture was diluted with H₂O (40.0 mL) and extracted with dichloromethane (40.0 mL*3). The combined organic layers were washed with brine (40.0 mL*3), dried over Na₂SO₄, filtered, and concentrated under reduced pressure to give a residue, which was purified by column chromatography (SiO₂, Petroleum ether:Ethyl acetate=1:0 to 0:1) to give benzyl ((1S)-(2-((5,5-difluoro-2-oxopiperidin-3-yl)methyl)imidazo[1,2-b][1,2,4]triazin-6-yl)(4,4-difluorocyclohexyl)methyl)carbamate (550 mg, 1.002 mmol, 41% yield over 4 steps) as a white solid. LCMS [M+H]⁺=549.3 m, z.

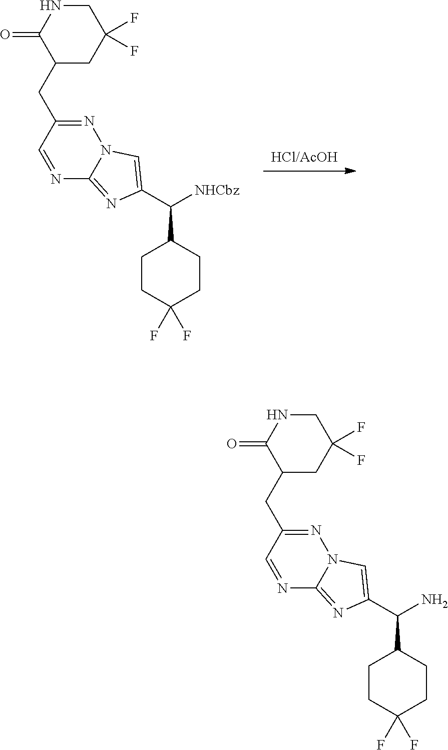 [SMILES: O=C(N[C@H](c1cn2nc(CC3CC(F)(F)CNC3=O)cnc2n1)C1CCC(F)(F)CC1)OCc1ccccc1 ; "HCl/AcOH" ; N[C@H](c1cn2nc(CC3CC(F)(F)CNC3=O)cnc2n1)C1CCC(F)(F)CC1]

To a solution of benzyl ((1S)-(2-((5,5-difluoro-2-oxopiperidin-3-yl)methyl)imidazo[1,2-b][1,2,4]triazin-6-yl)(4,4-difluorocyclohexyl)methyl)carbamate (170 mg, 309 μmol, 1.00 eq) in AcOH (0.50 mL) was added HCl (12.0 M, 5.67 mL, 219 eq). The mixture was stirred at room temperature for 0.25 h. After acid/base workup (ethyl acetate), the combined organic layers were dried over Na₂SO₄, filtered, and concentrated under reduced pressure to give 3-((6-((S)-amino(4,4-difluorocyclohexyl)methyl)imidazo[1,2-b][1,2,4]triazin-2-yl)methyl)-5,5-difluoropiperidin-2-one (0.09 g, crude) was obtained as a white solid. LCMS [M+H]⁺=415.3 m/z.

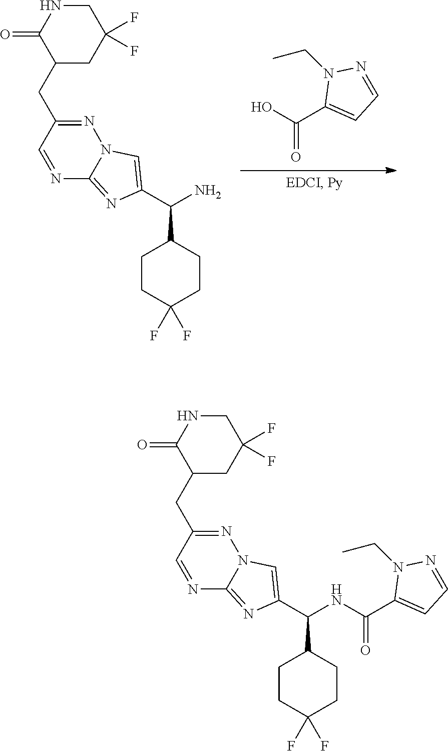

Amide coupling of 3-((6-((S)-amino(4,4-difluorocyclohexyl)methyl)imidazo[1,2-b][1,2,4]triazin-2-yl)methyl)-5,5-difluoropiperidin-2-one (90.0 mg, 217 μmol, 1.00 eq) with 1-ethyl-1H-pyrazole-5-carboxylic acid (45.6 mg, 325 μmol, 1.50 eq) using EDCI (124 mg, 651 μmol, 3.00 eq) in pyridine (1.00 mL) was carried out following the conditions in General Procedure 6. The product was purified by prep-TLC (SiO₂, dichloromethane:MeOH=10:1) then prep-SFC (column: DAICEL CHIRALPAK IC (250 mm*30 mm, 10 μm), mobile phase: [CO₂-EtOH (0.1% NH₃H₂O)]; B %: 35%, isocratic elution mode) to afford the second eluting single stereoisomer of N-((1S)-(2-((5,5-difluoro-2-oxopiperidin-3-yl)methyl)imidazo[1,2-b][1,2,4]triazin-6-yl)(4,4-difluorocyclohexyl)methyl)-1-ethyl-1H-pyrazole-5-carboxamide, Compound 63 (29.7 mg, 53.1 μmol, 56% yield) as a white solid. LCMS [M+H]⁺=537.3 nm/z.

### Example 84: Preparation of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-(2,2-difluorocyclopropyl)-1,2,5-oxadiazole-3-carboxamide (Compound 76 and 77 24114 and 24115)

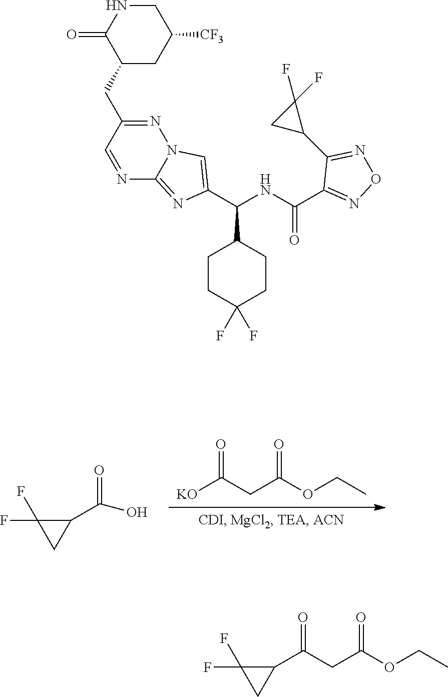

To a solution of 2,2-difluorocyclopropane-1-carboxylic acid (15.0 g, 123 mmol, 1.00 eq) in MeCN (90.0 mL) was added CDI (21.9 g, 135 mmol, 1.10 eq), and the solution was stirred for 1 h at 20° C. In a separate flask, TEA (37.3 g, 368 mmol, 51.3 mL, 3.00 eq) was added to a mixture of potassium 3-ethoxy-3-oxopropanoate (23.0 g, 135 mmol, 1.10 eq) and MgCl₂ (29.2 g, 307 mmol, 2.50 eq) in MeCN (400 mL). This mixture was stirred for 1 h at 20° C. The first solution was then added to the second mixture and the resulting mixture was heat to reflux at 80° C. for 2 h. The mixture was filtered and concentrated to remove solvent, then diluted with ethyl acetate (100 mL) and water (100 mL), extracted with ethyl acetate (100 mL*2), and the combined organic layers were washed with brine (200 mL), dried over Na₂SO₄, filtered, and concentrated to remove solvent. The crude material was purified by column chromatography (SiO₂, Petroleum Ether:ethyl acetate=1:0 to 10:1) to afford ethyl 3-(2,2-difluorocyclopropyl)-3-oxopropanoate (7.00 g, 36.4 mmol, 30% yield) as a yellow oil. ¹H NMR (400 MHz, CDCl₃) d 4.25-4.20 (m, 2H), 3.59 (s, 2H), 2.97-2.89 (m, 1H), 2.26-2.19 (m, 1H), 1.31-1.27 (t, J=7.4 Hz, 3H). ¹⁹F NMR (400 MHz, CDCl₃) d −124.5 (d, J=149 Hz, 1F), −140.0 (d, J=149 Hz, 1F).

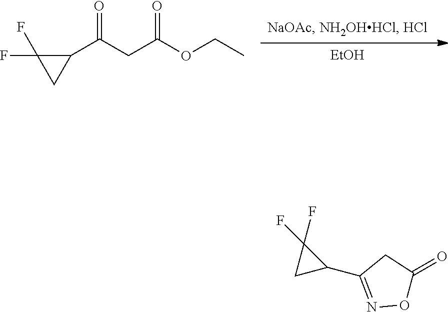

To a solution of ethyl 3-(2,2-difluorocyclopropyl)-3-oxopropanoate (7.00 g, 36.4 mmol, 1.00 eq) in EtOH (50.0 mL) was added NH₂OH·HCl (3.80 g, 54.6 mmol, 1.50 eq), NaOAc (4.48 g, 54.6 mmol, 1.50 eq), and HCl (6 M, 607 μL, 0.100 eq). The mixture was then stirred at 80° C. for 1 h, filtered, and concentrated to remove solvent, then diluted with water (50.0 mL) and extracted with ethyl acetate (50.0 mL*3). The combined organic layer was washed with brine (100 mL), dried over Na₂SO₄, filtered, and concentrated to remove solvent. The crude material was purified by column (SiO₂, Petroleum Ether:ethyl acetate=10:1 to 1:1) to afford 3-(2,2-difluorocyclopropyl)isoxazol-5(4H)-one (3.70 g, 22.9 mmol, 63% yield) as a yellow oil. ¹H NMR (400 MHz, CDCl₃) d 3.48-3.31 (m, 2H), 2.70-2.63 (m, 1H), 1.99-1.89 (m, 2H). LCMS [M+H]⁺=162.1 m/z.

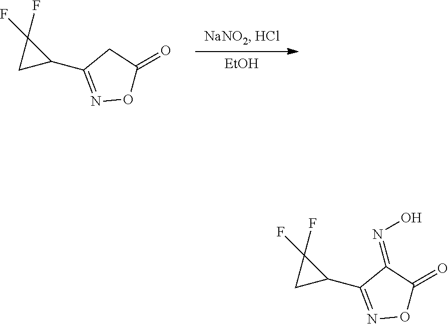

To a solution of 3-(2,2-difluorocyclopropyl)isoxazol-5(4H)-one (1.00 g, 6.21 mmol, 1.00 eq) in EtOH (10.0 mL) was added HCl (12 M, 620 μL, 1.20 eq) at 0° C. To the resulting solution was added a solution of NaNO₂ (642 mg, 9.31 mmol, 1.50 eq) in H₂O (1.00 mL). The mixture was stirred at 0° C. for 3 h, then diluted with water (20.0 mL), extracted with ethyl acetate (10.0 mL*3), and the combined organic layers were washed with brine (20.0 mL), dried over Na₂SO₄, filtered, and concentrated under reduced pressure to afford 3-(2,2-difluorocyclopropyl)-4-(hydroxyimino)isoxazol-5(4H)-one (1.00 g, 5.26 mmol, 85% yield) as a yellow solid. ¹H NMR (400 MHz, CDCl₃) d 3.32-3.04 (m, 1H), 2.21-2.04 (m, 2H).

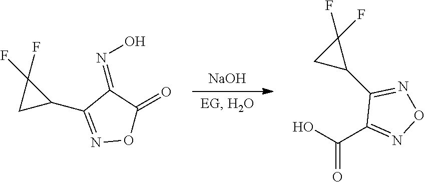

To a solution of 3-(2,2-difluorocyclopropyl)-4-(hydroxyimino)isoxazol-5(4H)-one (1.00 g, 5.26 mmol, 1.00 eq) in ethylene glycol (5.00 mL) and H₂O (1.00 mL) was added NaOH (1.05 g, 26.3 mmol, 5.00 eq) at 0° C. The mixture was stirred at 20° C. for 12 h then heated to 60° C. for 12 h. The mixture was diluted with water (20.0 mL) then washed with MTBE (10.0 mL*2). The aqueous phase was adjusted the pH=3 with 1M HCl, then extracted with ethyl acetate (10.0 mL*3). The combined organic layers were washed with brine (20.0 mL), dried over Na₂SO₄, filtered, and concentrated to remove solvent. The crude material was purified by column (SiO₂, Petroleum ether:ethyl acetate=10:1 to 1:1) to give racemic 4-(2,2-difluorocyclopropyl)-1,2,5-oxadiazole-3-carboxylic acid (130 mg, 683 μmol, 13% yield) as a yellow oil. ¹H NMR (400 MHz, CDCl₃) d 3.24-3.17 (m, 1H), 2.25-2.19 (m, 1H), 2.09-2.07 (m, 1H). ¹⁹F NMR (400 MHz, CDCl₃) d −128.5 (d, J=152 Hz, 1F), −141.5 (d, J=152 Hz, 1F).

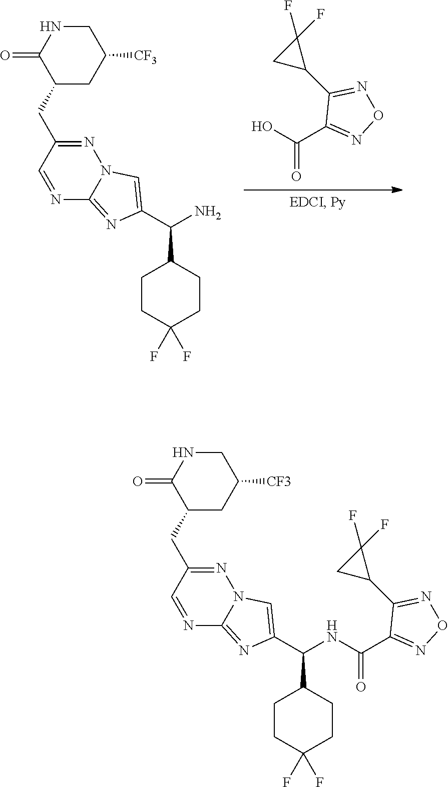

To a solution of (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one (60.0 mg, 134 μmol, 1.00 eq) and 4-(2,2-difluorocyclopropyl)-1,2,5-oxadiazole-3-carboxylic acid (76.6 mg, 403 μmol, 3.00 eq) in pyridine (2.00 mL) was added EDCI (51.5 mg, 268 μmol, 2.00 eq). The mixture was stirred at 20° C. for 2 h. then diluted with sat. aq. NaHCO₃ (5.00 mL) and water (5.00 mL) and extracted with ethyl acetate (5.00 mL*3). The combined organic layers were washed with brine (10.0 mL), dried over Na₂SO₄, filtered, and concentrated under reduced pressure to give a residue, which was purified by prep-TLC (SiO₂, dichloromethane:MeOH=10:1) to afford N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-(2,2-difluorocyclopropyl)-1,2,5-oxadiazole-3-carboxamide (30.0 mg, 48.5 μmol, 36% yield) as a white solid. Individual stereoisomers were separated by prep-SFC (column: DAICEL CHIRALPAK AD (250 mm*30 mm, 10 μm), mobile phase: [CO₂-i-PrOH (0.1% NH₃H₂O)]; B %: 30%, isocratic elution mode). The first eluting single stereoisomer of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-(2,2-difluorocyclopropyl)-1,2,5-oxadiazole-3-carboxamide, Compound 76 (6.78 mg, 9.53 μmol, 20% yield) was obtained as a yellow solid. LCMS [M+H]⁺=619.2 m/z.

The second eluting single stereoisomer of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-(2,2-difluorocyclopropyl)-1,2,5-oxadiazole-3-carboxamide, Compound 77 (8.19 mg, 12.98 μmol, 27% yield) was obtained as a yellow solid. LCMS [M+H]⁺=619.2 m/z.

### Example 85: Preparation of Individual Stereoisomers of benzyl ((1S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)carbamate

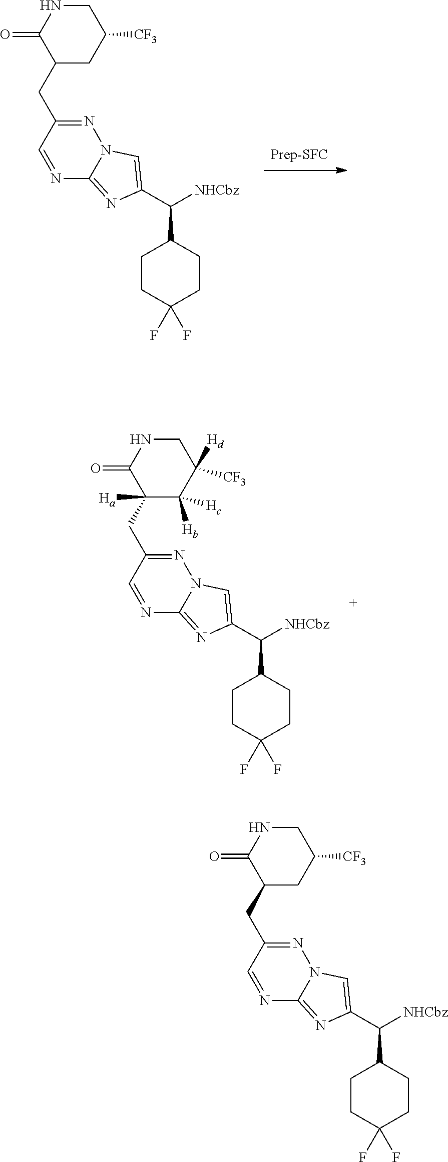

Single stereoisomers were separated by SFC (column: DAICEL CHIRALPAK IG (250 mm*30 mm, 10 μm); mobile phase: [CO₂-EtOH (0.100% NH₃·H₂O)]; B %: 60%, isocratic elution mode). The first eluting single stereoisomer, benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, was obtained as yellow solid. The relative stereochemistry was elucidated by 2D NMR, namely, utilizing HSQC δ (¹H, ¹³C): Hₐ (2.90, 33.5), H_{b} (2.23, 25.5), Hₑ (1.71, 25.5), and H_{d} (2.65, 38.0); and COSY for connectivity, showing Hₐ—H_{b}, Hₐ—Hₑ, H_{b}—H_{d}, and Hₑ—H_{d} correlations; and NOESY for stereochemical configuration: strong NOESY cross-peaks observed between Hₐ and H_{b} as well as H_{b} and H_{d}, but not between Hₐ and Hₑ nor between Hₑ and H_{d}. LCMS [M+H]⁺=581.2 m/z. The second eluting single stereoisomer, benzyl ((S)-(4,4-difluorocyclohexyl)(2-(((3S,5R)-2-oxo-5-(trifluoromethyl)piperidin-3-yl)methyl)imidazo[1,2-b][1,2,4]triazin-6-yl)methyl)carbamate, was obtained as yellow solid, with relative stereochemistry confirmed by deduction. LCMS [M+H]⁺=581.2 m/z.

### Example 86: Preparation of Individual Stereoisomers of benzyl ((1S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)carbamate

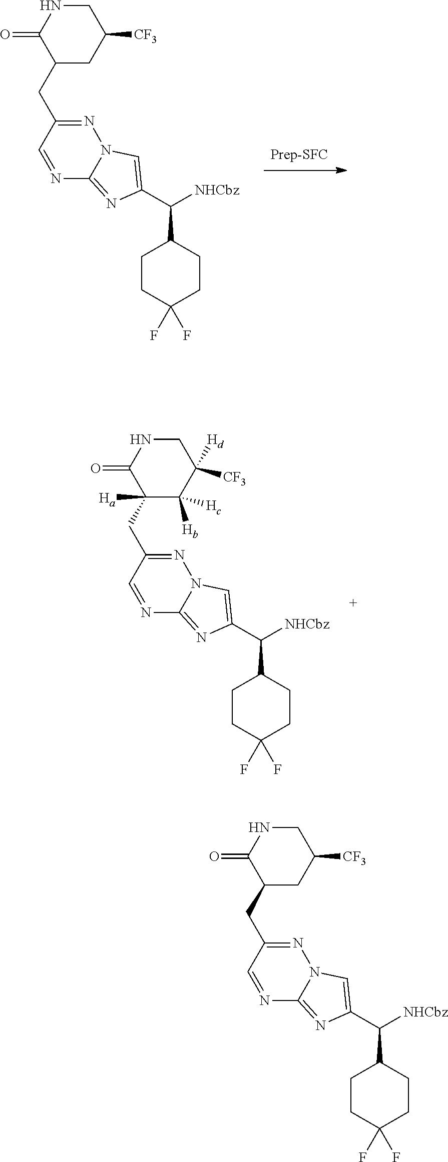

The synthesis of benzyl ((1S)-(4,4-difluorocyclohexyl)(2-(((5S)-2-oxo-5-(trifluoromethyl)piperidin-3-yl)methyl)imidazo[1,2-b][1,2,4]triazin-6-yl)methyl)carbamate was carried out in the same manner as benzyl ((1S)-(4,4-difluorocyclohexyl)(2-(((5R)-2-oxo-5-(trifluoromethyl)piperidin-3-yl)methyl)imidazo[1,2-b][1,2,4]triazin-6-yl)methyl)carbamate, with the only change being the substitution of 1-(tert-butyl) 3-methyl (5R)-2-oxo-5-(trifluoromethyl)piperidine-1,3-dicarboxylate with 1-(tert-butyl) 3-methyl (5S)-2-oxo-5-(trifluoromethyl)piperidine-1,3-dicarboxylate. Single stereoisomers were separated by SFC (column: DAICEL CHIRALPAK IG (250 mm*30 mm, 10 μm); mobile phase: [CO₂-EtOH (0.1% NH₃H₂O)]; B %: 50%, isocratic elution mode). The first eluting single stereoisomer, benzyl ((S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate, was obtained as white solid. The relative stereochemistry was elucidated by 2D NMR, namely, utilizing HSQC δ (¹H, ¹³C): Hₐ (3.12, 38.0), H_{b} (2.27, 25.5), H_{c} (1.99, 25.5), and H_{d} (2.78, 36.5); and COSY for connectivity, showing Hₐ—H_{b}, Hₐ—Hₑ, H_{b}—Hₐ, and H_{c}—H_{d} correlations; and NOESY for stereochemical configuration: strong NOESY cross-peaks observed between Hₐ and H_{b} as well as H_{c} and Hₐ, but not between Hₐ and H_{c} nor between H_{b} and Hₐ. LCMS [M+H]⁺=581.2 m/z. The second eluting single stereoisomer, benzyl ((S)-(4,4-difluorocyclohexyl)(2-(((3S,5S)-2-oxo-5-(trifluoromethyl)piperidin-3-yl)methyl)imidazo[1,2-b][1,2,4]triazin-6-yl)methyl)carbamate, was obtained as white solid, with relative stereochemistry confirmed by deduction. LCMS [M+H]⁺=581.2 m/z.

### Example 87: Preparation of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 66)

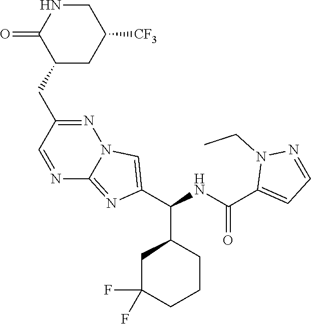 [SMILES: CCn1nccc1C(=O)N[C@H](c1cn2nc(C[C@H]3C[C@@H](C(F)(F)F)CNC3=O)cnc2n1)[C@H]1CCCC(F)(F)C1]

N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((S)-3,3-difluorocyclohexyl)-2-oxopropyl)carbamate, General Procedure 5, employing benzyl ((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

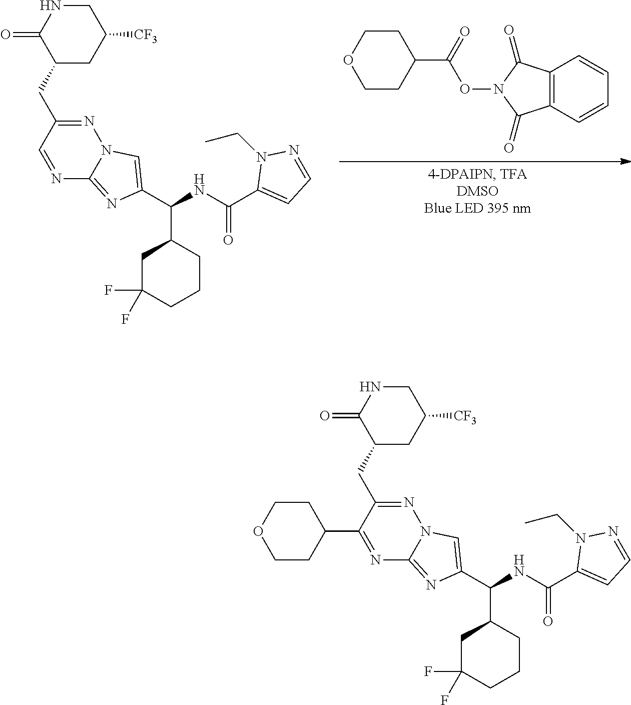 [SMILES: CCn1nccc1C(=O)N[C@H](c1cn2nc(C[C@H]3C[C@@H](C(F)(F)F)CNC3=O)cnc2n1)[C@H]1CCCC(F)(F)C1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]

To a solution of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (80 mg, 140 μmol, 1.00 eq) and 1,3-dioxoisoindolin-2-yl tetrahydro-2H-pyran-4-carboxylate (96.8 mg, 351 μmol, 2.50 eq) in DMSO (3.00 mL) was added 4-DPAIPN (5.61 mg, 7.04 μmol, 0.0500 eq) and TFA (32.0 mg, 281 μmol, 20.9 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 40%-60% B over 10 min) to afford N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-11H-pyrazole-5-carboxamide, Compound 66 (56.85 mg, 85.01 μmol, 60.42% yield) as a yellow solid. LCMS [M+H]⁺=653.5 m/z.

### Example 88: Preparation of N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (Compounds 68 and 70)

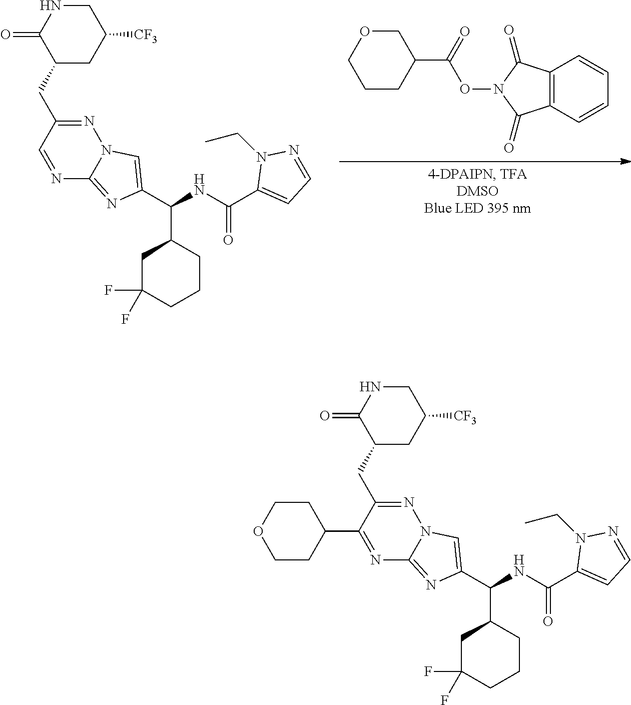 [SMILES: CCn1nccc1C(=O)N[C@H](c1cn2nc(C[C@H]3C[C@@H](C(F)(F)F)CNC3=O)cnc2n1)[C@H]1CCCC(F)(F)C1 ; O=C(ON1C(=O)c2ccccc2C1=O)C1CCCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]

To a solution of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (140 mg, 246 μmol, 1.00 eq) and 1,3-dioxoisoindolin-2-yl tetrahydro-2H-pyran-3-carboxylate (169 mg, 615 μmol, 2.50 eq) in DMSO (3.00 mL) was added 4-DPAIPN (9.81 mg, 12.3 μmol, 0.05 eq) and TFA (56.1 mg, 492 μmol, 36.5 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 41%-61% B over 10 min) to afford N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (0.075 g, 114 mol, 46.6% yield) as a yellow solid. LCMS [M+H]⁺=653.3 m/z.

Compound 68 was isolated as the first eluting, single stereoisomer by chiral SFC purification (column: Daicel Chiralcel OD (250 mm×30 mm, 10 μm); mobile phase: [CO₂-MeOH]; B %:25%, isocratic elution mode); (31.45 mg, 47.46 μmol, 41.30% yield) and was obtained as a yellow solid. LCMS [M+H]⁺=653.2 m/z.

Compound 70 was isolated as the second eluting, single stereoisomer by chiral SFC purification (column: Daicel Chiralcel OD (250 mm×30 mm, 10 μm); mobile phase: [CO₂-MeOH]; B %:25%, isocratic elution mode); (38.88 mg, 59.27 μmol, 51.58% yield) and was obtained as a yellow solid. LCMS [M+H]⁺=653.2 m/z.

### Example 89: Preparation of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 67)

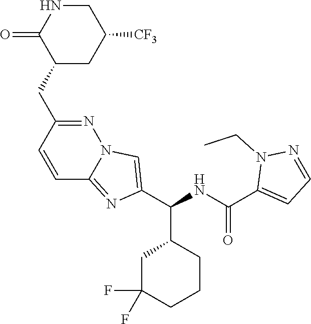 [SMILES: CCn1nccc1C(=O)N[C@H](c1cn2nc(C[C@H]3C[C@@H](C(F)(F)F)CNC3=O)ccc2n1)[C@@H]1CCCC(F)(F)C1]

N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((R)-3,3-difluorocyclohexyl)-2-oxopropyl)carbamate, General Procedure 5, employing benzyl ((1S)—((R)-3.3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

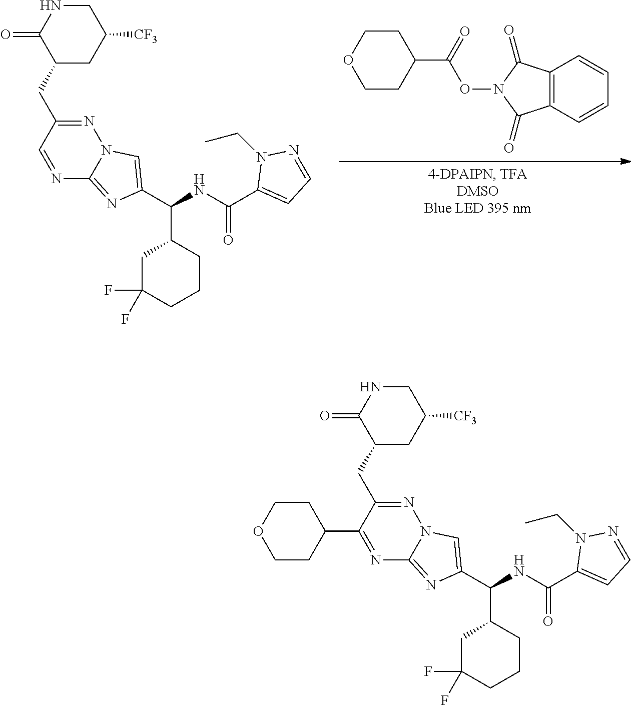 [SMILES: CCn1nccc1C(=O)N[C@H](c1cn2nc(C[C@H]3C[C@@H](C(F)(F)F)CNC3=O)cnc2n1)[C@@H]1CCCC(F)(F)C1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]

To a solution of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (90.0 mg, 158 μmol, 1.00 eq) and 1,3-dioxoisoindolin-2-yl tetrahydro-2H-pyran-4-carboxylate (87.1 mg, 316 μmol, 2.00 eq) in DMSO (3.00 mL) was added 4-DPAIPN (6.31 mg, 7.91 μmol, 0.05 eq) and TFA (36.1 mg, 316 μmol, 23.5 μL, 2.00 eq). The vial was sealed and placed under nitrogen. The reaction mixture was stirred and irradiated with a 395 nm LED lamp, with cooling fan to keep the reaction temperature at room temperature for 16 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 43%-73% B over 10 min) and chiral SFC purification (column: Daicel Chiralpak AD (250 mm×30 mm, 10 μm); mobile phase: [CO₂-EtOH (0.1% NH₃H₂O)]; B %:30%, isocratic elution mode) to afford N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide, Compound 67 (37.82 mg, 57.4 μmol, 36.3% yield) as a yellow solid. LCMS [M+H]⁺=653.3 m/z.

### Example 90: Preparation of N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (Compounds 69 and 71)

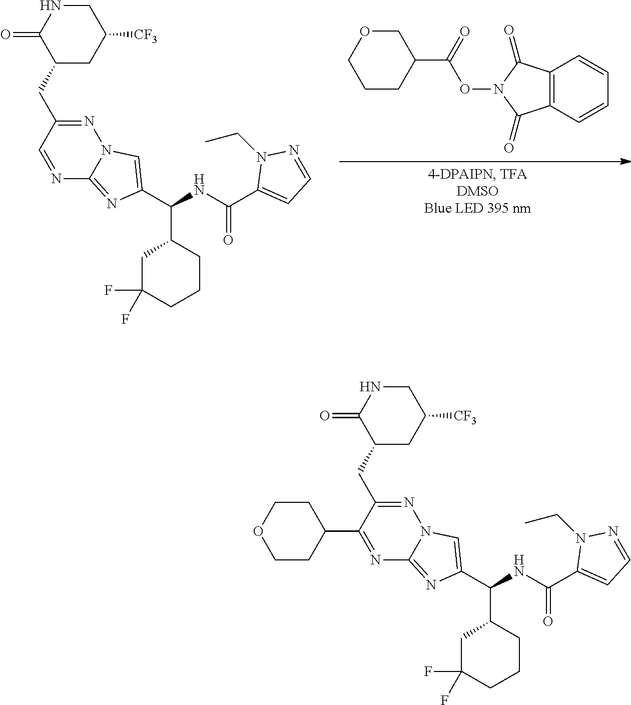 [SMILES: CCn1nccc1C(=O)N[C@H](c1cn2nc(C[C@H]3C[C@@H](C(F)(F)F)CNC3=O)cnc2n1)[C@@H]1CCCC(F)(F)C1 ; O=C(ON1C(=O)c2ccccc2C1=O)C1CCCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]

To a solution of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (150 mg, 263 μmol, 1.00 eq) and 1,3-dioxoisoindolin-2-yl tetrahydro-2H-pyran-3-carboxylate (145 mg, 527 μmol, 2.00 eq) in DMSO (4.00 mL) was added 4-DPAIPN (10.5 mg, 13.1 μmol, 0.05 eq) and TFA (60.1 mg, 527 μmol, 39.2 μL, 2.00 eq). The vial was sealed and placed under nitrogen. The reaction mixture was stirred and irradiated with a 395 nm LED lamp, with cooling fan to keep the reaction temperature at room temperature for 16 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 47%-77% B over 10 min) to afford N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (90.0 mg, 137 μmol, 52.3% yield) as a yellow solid. LCMS [M+H]⁺=653.3 m/z.

Compound 69 was isolated as the first eluting, single stereoisomer by chiral SFC purification (column: Daicel Chiralcel OD-H (250 mm×30 mm, 5 μm); mobile phase: [CO₂-MeOH (0.1% NH₃H₂O)]; B %: 30%, isocratic elution mode); (38.89 mg, 59.2 μmol, 25.8% yield) and was obtained as a white solid. LCMS [M+H]⁺=653.3 m/z.

Compound 71 was isolated as the second eluting, single stereoisomer by chiral SFC purification (column: Daicel Chiralcel OD-H (250 mm×30 mm, 5 um); mobile phase: [CO₂-MeOH (0.1% NH₃H₂O)]; B %: 30%, isocratic elution mode); (36.14 mg, 55.2 μmol, 24.1% yield) and was obtained as a white solid. LCMS [M+H]⁺=653.3 m/z.

### Example 91: Preparation of N-((1S)—((S)-3,3-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide (Compounds 203 and 204)

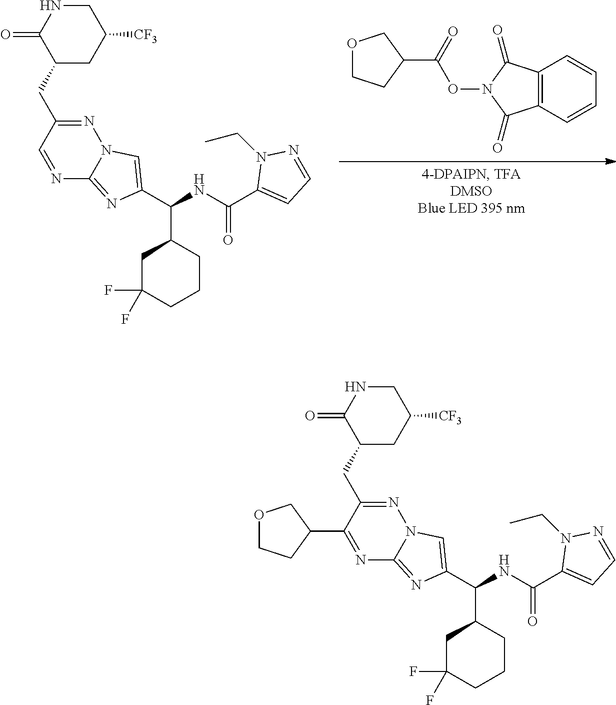 [SMILES: CCn1nccc1C(=O)N[C@H](c1cn2nc(C[C@H]3C[C@@H](C(F)(F)F)CNC3=O)cnc2n1)[C@H]1CCCC(F)(F)C1 ; O=C(ON1C(=O)c2ccccc2C1=O)C1CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC3)nc2n1)[C@H]1CCCC(F)(F)C1]

To a solution of N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq) and 1,3-dioxoisoindolin-2-yl tetrahydrofuran-3-carboxylate (91.8 mg, 351 μmol, 2.00 eq) in DMSO (2.00 mL) was added 4-DPAIPN (7.01 mg, 8.79 μmol, 0.05 eq) and TFA (40.1 mg, 351 μmol, 26.1 μL, 2.00 eq). The vial was sealed and placed under nitrogen. The reaction mixture was stirred and irradiated with a 395 nm LED lamp, with cooling fan to keep the reaction temperature at room temperature for 16 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 38%-65% B over 9 min) to afford N-((1S)—((S)-3,3-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide (80.0 mg, 125 μmol, 71.2% yield) as a yellow solid. LCMS [M+H]⁺=639.3 m/z.

Compound 203 was isolated as the first eluting, single stereoisomer by chiral SFC purification (column: Daicel Chiralcel OD (250 mm×30 mm, 10 um); mobile phase: [Hexane-IPA]: B %: 30%, isocratic elution mode); (42.33 mg, 65.4 μmol, 52.2% yield) and was obtained as a white solid. LCMS [M+H]⁺=639.2 m/z.

Compound 204 was isolated as the second eluting, single stereoisomer by chiral SFC purification (column: Daicel Chiralcel OD (250 mm×30 mm, 10 μm); mobile phase: [Hexane-IPA]; B %:30%, isocratic elution mode); (56.34 mg, 86.3 μmol, 68.9% yield) and was obtained as a white solid. LCMS [M+H]⁺=639.3 m/z.

### Example 92: Preparation of N-((1S)—((R)-3,3-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide (Compounds 72 and 73)

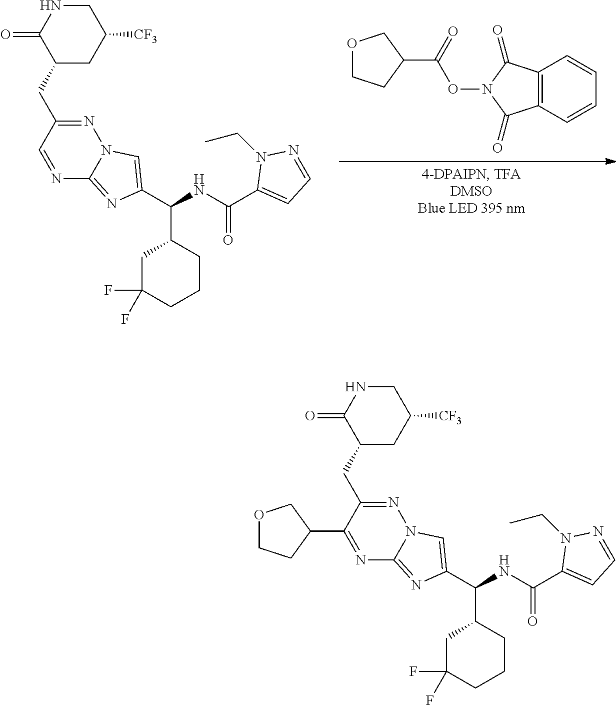 [SMILES: CCn1nccc1C(=O)N[C@H](c1cn2nc(C[C@H]3C[C@@H](C(F)(F)F)CNC3=O)cnc2n1)[C@@H]1CCCC(F)(F)C1 ; O=C(ON1C(=O)c2ccccc2C1=O)C1CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC3)nc2n1)[C@@H]1CCCC(F)(F)C1]

To a solution of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (0.200 g, 351 μmol, 1.00 eq) and 1,3-dioxoisoindolin-2-yl tetrahydrofuran-3-carboxylate (183 mg, 703 μmol, 2.00 eq) in DMSO (3.00 mL) was added 4-DPAIPN (14.0 mg, 17.5 μmol, 0.0500 eq) and TFA (80.2 mg, 703 μmol, 52.2 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by chiral SFC purification (column: Daicel Chiralcel OX (250 mm×30 mm, 10 μm); mobile phase: [CO₂-MeOH (0.1% NH₃H₂O)]; B %:35%, isocratic elution mode) to afford N-((1S)—((R)-3,3-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide (0.100 g, 156 μmol, 44.5% yield) as a yellow solid. LCMS [M+H]⁺=639.3 m/z.

Compound 72 was isolated as the first eluting, single stereoisomer by chiral SFC purification (column: Daicel Chiralcel OD (250 mm×30 mm, 10 μm); mobile phase: [CO₂-MeOH (0.1% NH₃H₂O)]; B %:40%, isocratic elution mode); (26.35 mg, 39.94 μmol, 25.5% yield) and was obtained as a yellow solid. LCMS [M+H]⁺=639.4 m/z.

Compound 73 was isolated as the second eluting, single stereoisomer by chiral SFC purification (column: Daicel Chiralcel OD (250 mm×30 mm, 10 μm); mobile phase: [CO₂-MeOH (0.1% NH₃H₂O)]; B %:40%, isocratic elution mode); (32.0 mg, 49.26 μmol, 31.4% yield) and was obtained as a yellow solid. LCMS [M+H]⁺=639.4 m/z.

### Example 93: Preparation of N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (Compounds 74 and 75)

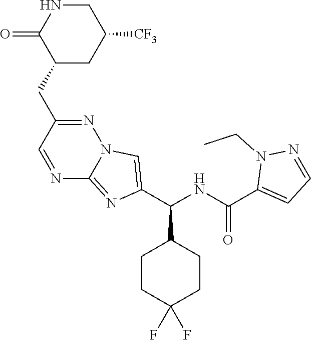 [SMILES: CCn1nccc1C(=O)N[C@H](c1cn2nc(C[C@H]3C[C@@H](C(F)(F)F)CNC3=O)cnc2n1)C1CCC(F)(F)CC1]

N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 5, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

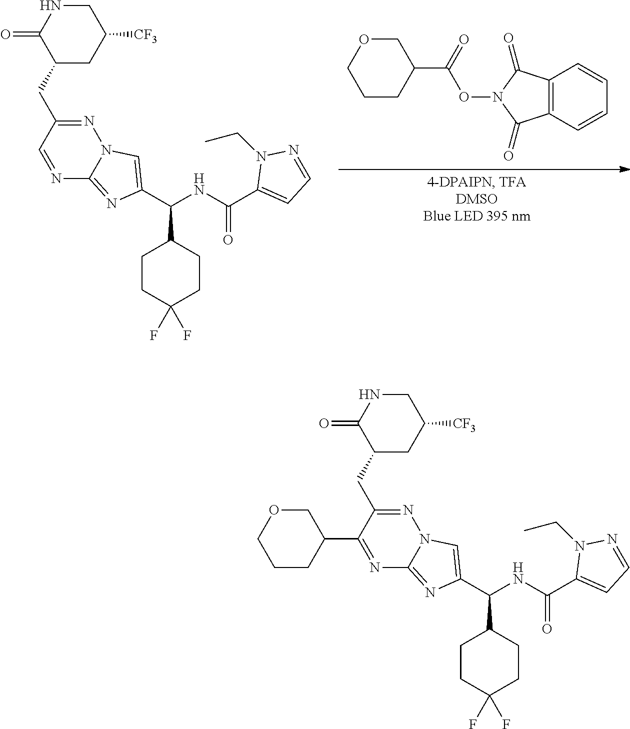 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCCOC3)nc2n1)C1CCC(F)(F)CC1]

To a solution of N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (110 mg, 193 μmol, 1.00 eq) and 1,3-dioxoisoindolin-2-yl tetrahydro-2H-pyran-3-carboxylate (106 mg, 386 μmol, 2.00 eq) in DMSO (3.00 mL) was added 4-DPAIPN (7.71 mg, 9.67 μmol, 0.050 eq) and TFA (44.1 mg, 386 μmol, 28.7 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm×10 μm; mobile phase: [water (FA)—acetonitrile]; gradient: 40%-70% B over 10 min) to afford N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-ethyl-1H-pyrazole-5-carboxamide (100 mg, 153 μmol, 79.2% yield) as a yellow solid. LCMS [M+H]⁺=653.4 m/z.

Compound 74 was isolated as the first eluting, single stereoisomer by chiral SFC purification (column: Daicel Chiralpak AD (250 mm×30 mm, 10 μm); mobile phase: [CO₂-i-PrOH (0.1% NH₃H₂O)]; B %: 25%, isocratic elution mode); (13.6 mg, 20.7 μmol, 13.5% yield) and was obtained as a white solid. LCMS [M+H]⁺=653.4 m/z.

Compound 75 was isolated as the second eluting, single stereoisomer by chiral SFC purification (column: Daicel Chiralpak AD (250 mm×30 mm, 10 μm); mobile phase: [CO₂-i-PrOH (0.1% NH₃H₂O)]; B %: 25%, isocratic elution mode); (29.7 mg, 45.6 μmol, 29.7% yield) and was obtained as a white solid. LCMS [M+H]⁺=653.4 m/z.

### Example 94: Preparation of 1-ethyl-N-((1S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (Compound 78)

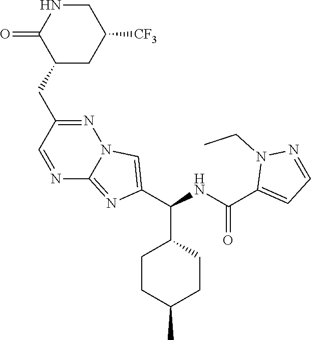 [SMILES: CCn1nccc1C(=O)N[C@H](c1cn2nc(C[C@H]3C[C@@H](C(F)(F)F)CNC3=O)cnc2n1)[C@H]1CC[C@H](C)CC1]

1-Ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((1R,4S)-4-methylcyclohexyl)-2-oxopropyl)carbamate, General Procedure 5, employing benzyl ((1S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((1r,4S)-4-methylcyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

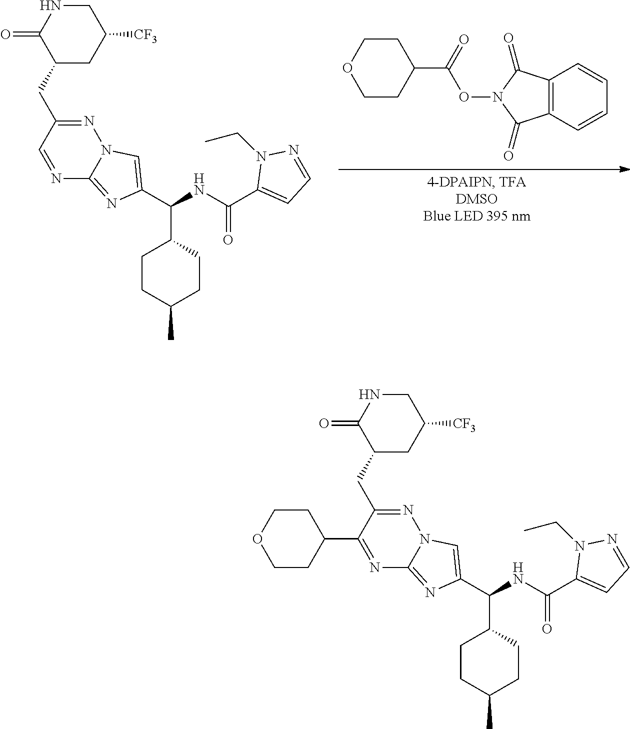 [SMILES: CCn1nccc1C(=O)N[C@H](c1cn2nc(C[C@H]3C[C@@H](C(F)(F)F)CNC3=O)cnc2n1)[C@H]1CC[C@H](C)CC1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)[C@H]1CC[C@H](C)CC1]

To a solution of 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (100 mg, 183 μmol, 1.00 eq) and 1,3-dioxoisoindolin-2-yl tetrahydro-2H-pyran-4-carboxylate (101 mg, 366 μmol, 2.00 eq) in DMSO (3.00 mL) was added 4-DPAIPN (7.29 mg, 9.15 mol, 0.0500 eq) and TFA (41.7 mg, 366 μmol, 27.2 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm, 10 μm; mobile phase: [water (FA)—acetonitrile]; gradient: 52%-82% B over 10 min) to afford 1-ethyl-N-((1S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide, Compound 78 (43.8 mg, 68.0 μmol, 37.1% yield) as a yellow solid. LCMS [M+H]⁺=631.5 m/z.

### Example 95: Preparation of 1-ethyl-N-((1S)-(3-isopropyl-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1H-pyrazole-5-carboxamide (Compound 110)

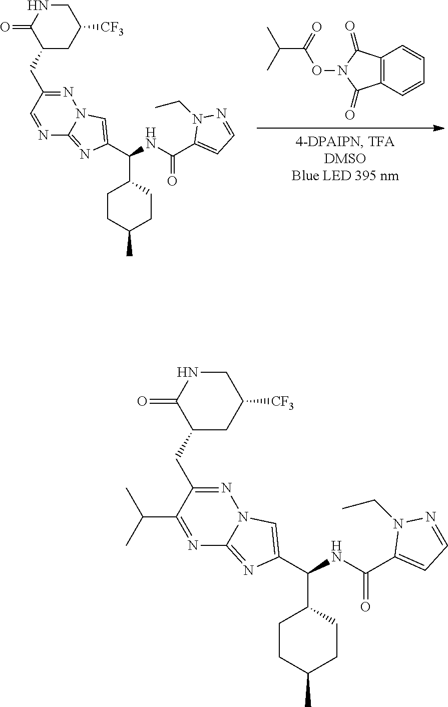 [SMILES: CCn1nccc1C(=O)N[C@H](c1cn2nc(C[C@H]3C[C@@H](C(F)(F)F)CNC3=O)cnc2n1)[C@H]1CC[C@H](C)CC1 ; CC(C)C(=O)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)C)nc2n1)[C@H]1CC[C@H](C)CC1]

To a solution of 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (100 mg, 183 μmol, 1.00 eq) and 1,3-dioxoisoindolin-2-yl isobutyrate (85.3 mg, 366 μmol, 2.00 eq) in DMSO (3.00 mL) was added 4-DPAIPN (7.29 mg, 9.15 μmol, 0.0500 eq) and TFA (41.7 mg, 366 μmol, 27.2 μL, 2.00 eq). The vial was sealed and placed under nitrogen. The reaction mixture was stirred and irradiated with a 395 nm LED lamp, with cooling fan to keep the reaction temperature at room temperature for 14 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm, 10 μm; mobile phase: [water (FA)—acetonitrile]; gradient: 52%-72% B over 10 min) to afford 1-ethyl-N-((1S)-(3-isopropyl-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1H-pyrazole-5-carboxamide, Compound 110 (47.6 mg, 79.4 μmol, 43.4% yield) as a yellow solid. LCMS [M+H]⁺=589.5 m/z.

### Example 96: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 88)

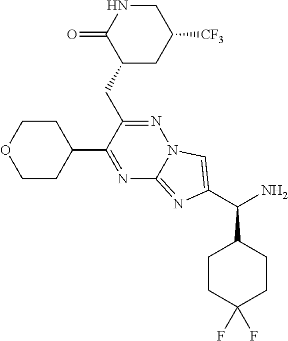 [SMILES: N[C@H](c1cn2nc(C[C@H]3C[C@@H](C(F)(F)F)CNC3=O)c(C3CCOCC3)nc2n1)C1CCC(F)(F)CC1]

(3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate and 1,3-dioxoisoindolin-2-yl tetrahydro-2H-pyran-4-carboxylate, and General Procedure 4, employing benzyl ((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate.

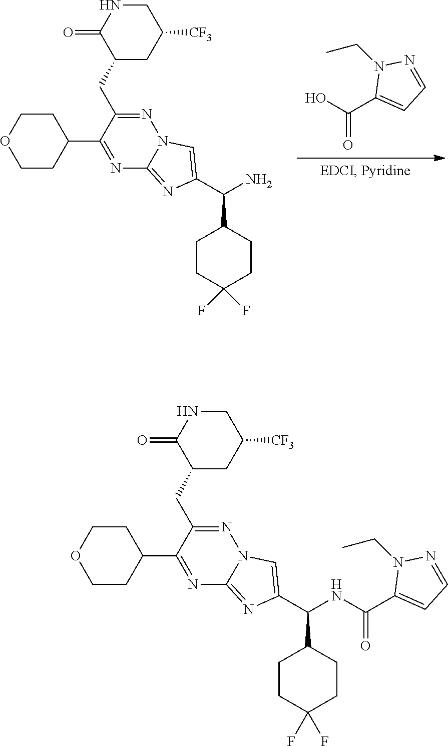

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 1-ethyl-1H-pyrazole-5-carboxylic acid (12.6 mg, 90.4 μmol, 1.20 eq) and was purified by prep-TLC (SiO2, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide Compound 88 (25.60 mg, 38.6 μmol, 51.2% yield) as a white solid. LCMS [M+H]⁺=653.4 m/z.

### Example 97: Preparation of 4-cyclopropyl-N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1,2,5-oxadiazole-3-carboxamide (Compound 136)

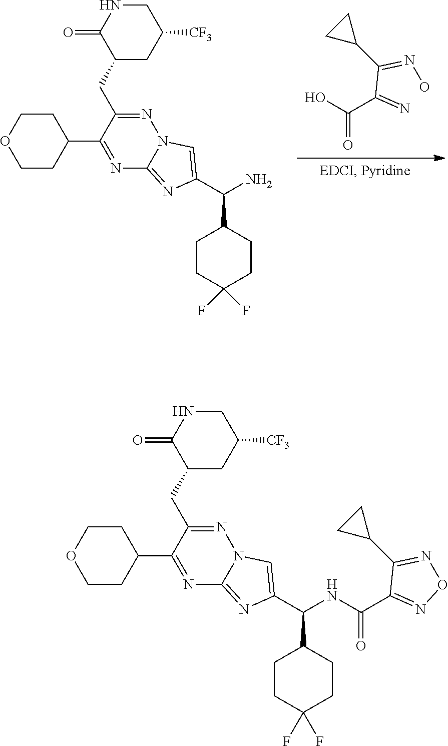

4-Cyclopropyl-N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1,2,5-oxadiazole-3-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 mol, 1.00 eq), 4-cyclopropyl-1,2,5-oxadiazole-3-carboxylic acid (13.9 mg, 90.4 μmol, 1.20 eq) and was purified by prep-TLC (SiO2, dichloromethane:methanol) to afford 4-cyclopropyl-N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1,2,5-oxadiazole-3-carboxamide, Compound 136 (20.91 mg, 31.21 μmol, 41.39% yield) as a yellow solid. LCMS [M+H]⁺=667.5 m/z.

### Example 98: Preparation of 4 N-((1S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 87)

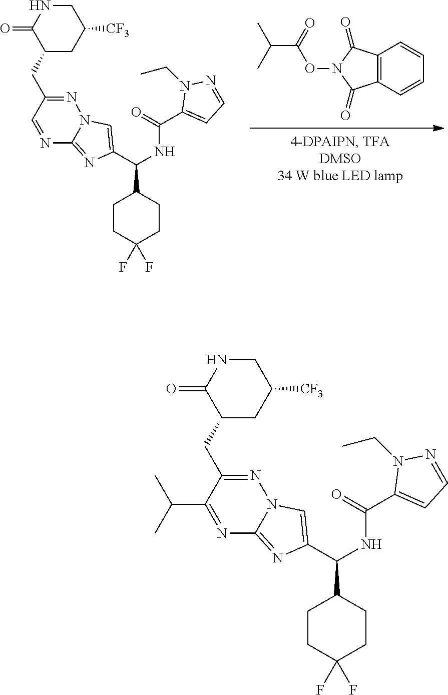 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)C(=O)ON1C(=O)c2ccccc2C1=O ; "4-DPAIPN, TFADMSO34 W blue LED lamp" ; CCn1nccc1C(=O)N[C@H](c1cn2nc(C[C@H]3C[C@@H](C(F)(F)F)CNC3=O)c(C(C)C)nc2n1)C1CCC(F)(F)CC1]

N-((1S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (113 mg, 0.200 mmol, 1.00 eq), 1,3-dioxoisoindolin-2-yl isobutyrate (69.9 mg, 300 μmol, 1.50 eq) and was purified by prep-HPLC (column: Phenomenex luna C18 150×25 mm, 10 μm; mobile phase: [water (FA)—acetonitrile]; gradient: 46%-76% B over 10 min) to afford 4 N-((1S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 87 (53.16 mg, 83.23 μmol, 41.61% yield) as a white solid. LCMS [M+H]⁺=611.4 m/z.

### Example 99: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 89)

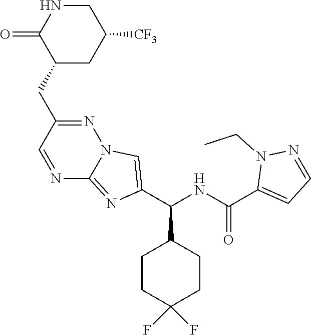 [SMILES: CCn1nccc1C(=O)N[C@H](c1cn2nc(C[C@H]3C[C@@H](C(F)(F)F)CNC3=O)cnc2n1)C1CCC(F)(F)CC1]

N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 5, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

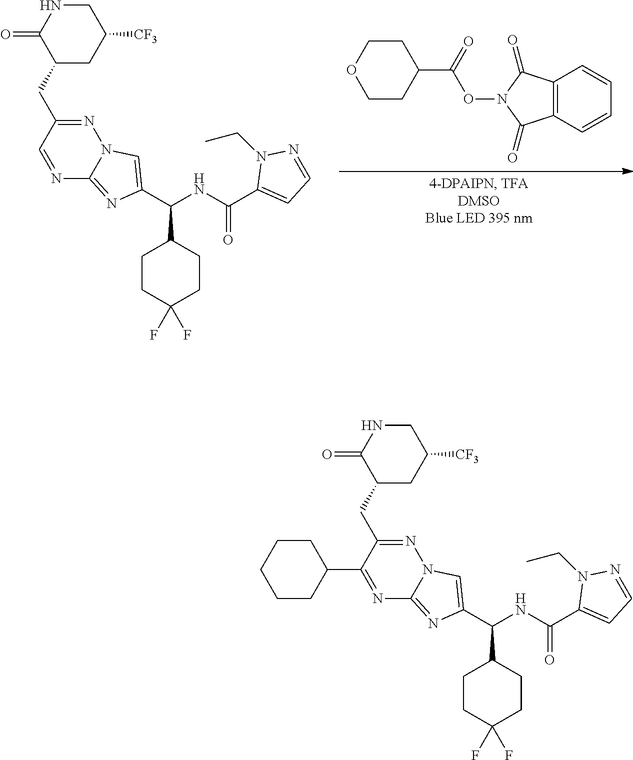 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCCCC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (80.0 mg, 140 μmol, 1.00 eq), 1,3-dioxoisoindolin-2-yl tetrahydro-2H-pyran-4-carboxylate (77.4 mg, 281 μmol, 2.00 eq) and was purified by prep-HPLC (column: Phenomenex luna C18 150×25 mm, 10 μm; mobile phase: [water (FA)—acetonitrile]; gradient: 36%-66% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide, Compound 89 (44.44 mg, 65.0 μmol, 46.2% yield) as a white solid. LCMS [M+H]⁺=653.4 m/z.

### Example 100: Preparation of N-((1S)—((S)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 205)

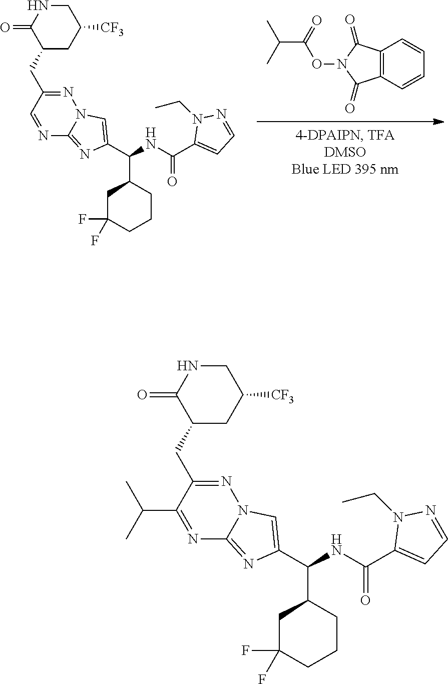 [SMILES: CCn1nccc1C(=O)N[C@H](c1cn2nc(C[C@H]3C[C@@H](C(F)(F)F)CNC3=O)cnc2n1)[C@H]1CCCC(F)(F)C1 ; CC(C)C(=O)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)C)nc2n1)[C@H]1CCCC(F)(F)C1]

N-((1S)—((S)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (95.0 mg, 167 μmol, 1.00 eq), 1,3-dioxoisoindolin-2-yl isobutyrate (77.9 mg, 334 μmol, 2.00 eq) and was purified by prep-HPLC (column: Phenomenex luna C18 150×25 mm, 10 μm; mobile phase: [water (FA)—acetonitrile], gradient: 42%-72% B over 10 min) to afford N-((1S)—((S)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 205 (48.2 mg, 77.9 μmol, 46.6% yield) as a yellow solid. LCMS [M+H]⁺=611.3 m/z.

### Example 101: Preparation of N-((1S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide (Compound 211)

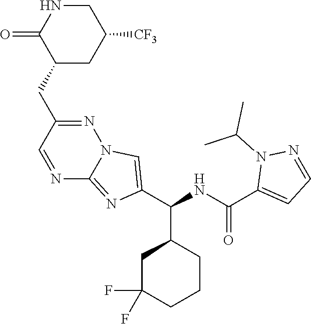 [SMILES: CC(C)n1nccc1C(=O)N[C@H](c1cn2nc(C[C@H]3C[C@@H](C(F)(F)F)CNC3=O)cnc2n1)[C@H]1CCCC(F)(F)C1]

N-((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((S)-3,3-difluorocyclohexyl)-2-oxopropyl)carbamate, General Procedure 5, employing benzyl ((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one and 1-isopropyl-1H-pyrazole-5-carboxylic acid.

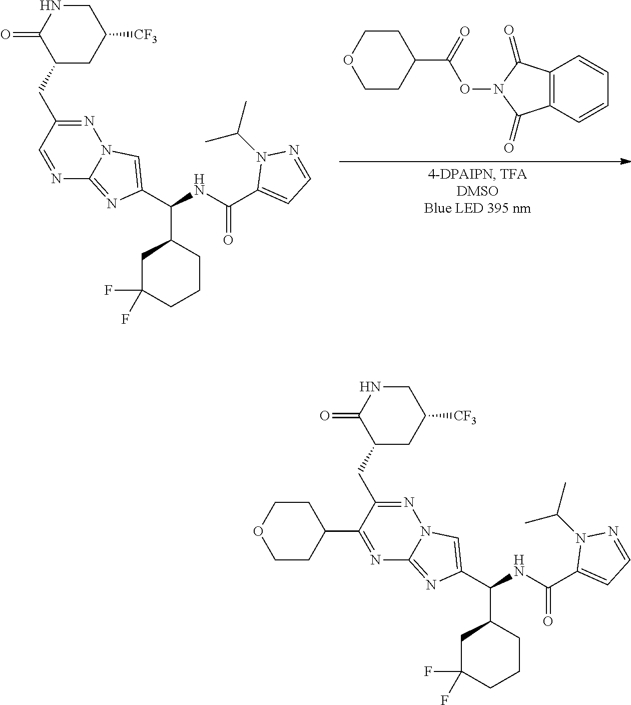 [SMILES: CC(C)n1nccc1C(=O)N[C@H](c1cn2nc(C[C@H]3C[C@@H](C(F)(F)F)CNC3=O)cnc2n1)[C@H]1CCCC(F)(F)C1 ; O=C(ON1C(=O)c2ccccc2C1=O)C1CCOCC1 ; "4-DPAIPN, TFADMSOBlue LED 395 nm" ; CC(C)n1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]

N-((1S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide was synthesized following the General Procedure 7 with N-((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 171 μmol, 1.00 eq), 1,3-dioxoisoindolin-2-yl tetrahydro-2H-pyran-4-carboxylate (94.5 mg, 343 μmol, 2.00 eq) and was purified by prep-HPLC (column: Phenomenex luna C18 150×25 mm, 10 μm; mobile phase: [water (FA)—acetonitrile]; gradient: 44%-64% B over 10 min) to afford N-((1S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide, Compound 211 (57.0 mg, 84.1 μmol, 49.0% yield) as a yellow solid. LCMS [M+H]⁺=667.3 m/z.

### Example 102: Preparation of 4-Cyclopropyl-N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1,2,5-oxadiazole-3-carboxamide (Compounds 125 and 126)

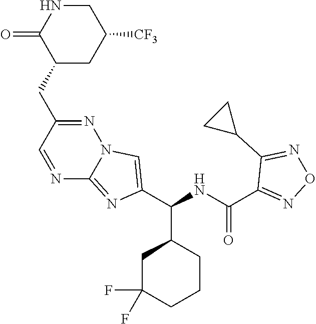 [SMILES: O=C(N[C@H](c1cn2nc(C[C@H]3C[C@@H](C(F)(F)F)CNC3=O)cnc2n1)[C@H]1CCCC(F)(F)C1)c1nonc1C1CC1]

4-Cyclopropyl-N-((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((S)-3,3-difluorocyclohexyl)-2-oxopropyl)carbamate, General Procedure 5, employing benzyl ((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((S)-3,3-difluorocyclohexyl)methyl)imidazo[1,2-b][1,2,4]triazin-2-yl)methyl)-5-(trifluoromethyl)piperidin-2-one and 4-cyclopropyl-1,2,5-oxadiazole-3-carboxylic acid.

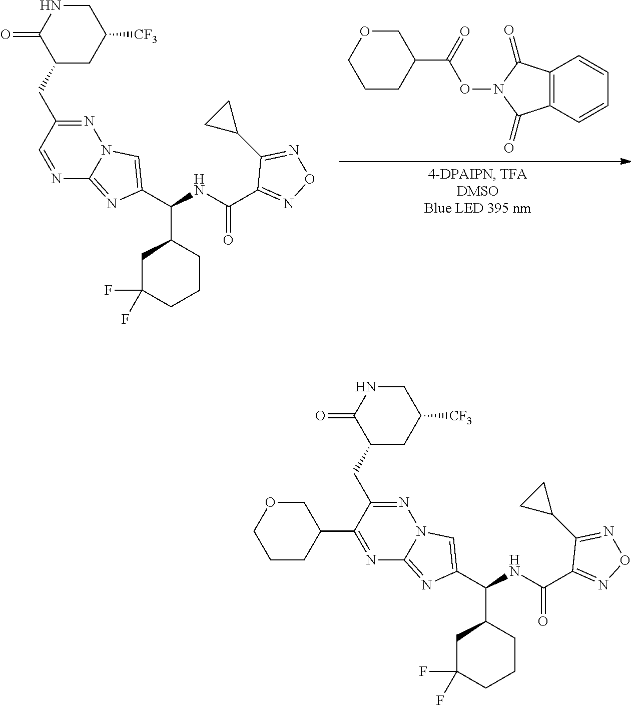 [SMILES: O=C(N[C@H](c1cn2nc(C[C@H]3C[C@@H](C(F)(F)F)CNC3=O)cnc2n1)[C@H]1CCCC(F)(F)C1)c1nonc1C1CC1 ; O=C(ON1C(=O)c2ccccc2C1=O)C1CCCOC1 ; "4-DPAIPN, TFADMSOBlue LED 395 nm" ; O=C(N[C@H](c1cn2nc(C[C@H]3C[C@@H](C(F)(F)F)CNC3=O)c(C3CCCOC3)nc2n1)[C@H]1CCCC(F)(F)C1)c1nonc1C1CC1]

4-Cyclopropyl-N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl-1,2,5-oxadiazole-3-carboxamide was synthesized following the General Procedure 7 with 4-cyclopropyl-N-((1S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (180 mg, 309 μmol, 1.00 eq), 1,3-dioxoisoindolin-2-yl tetrahydro-2H-pyran-3-carboxylate (170 mg, 618 μmol, 2.00 eq) and was purified by prep-HPLC (column: Phenomenex luna C18 150×25 mm, 10 μm; mobile phase: [water (FA)—acetonitrile]; gradient: 52%-72% B over 10 min) to afford 4-Cyclopropyl-N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1,2,5-oxadiazole-3-carboxamide (70.0 mg, 105 μmol, 33.9% yield) as a yellow solid. LCMS [M+H]⁺=667.3 m/z.

Compound 125 was isolated as the first eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 35%, isocratic elution mode); (22.5 mg, 33.1 μmol, 31.5% yield) and was obtained as a yellow solid. LCM [M+H]⁺=667.5 m/z.

Compound 126 was isolated as the second eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 35%, isocratic elution mode); (32.9 mg, 47.5 μmol, 45.2% yield) and was obtained as a yellow solid. LCMS [M+H]⁺=667.5 m/z.

### Example 103: Preparation of 4-Cyclopropyl-N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1,2,5-oxadiazole-3-carboxamide (Compounds 209 and 210)

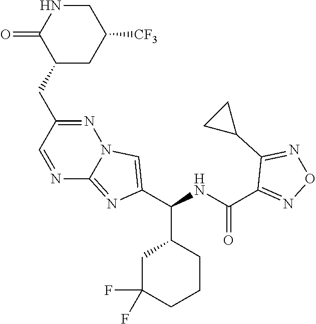 [SMILES: O=C(N[C@H](c1cn2nc(C[C@H]3C[C@@H](C(F)(F)F)CNC3=O)cnc2n1)[C@@H]1CCCC(F)(F)C1)c1nonc1C1CC1]

4-Cyclopropyl-N-((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((R)-3,3-difluorocyclohexyl)-2-oxopropyl)carbamate, General Procedure 5, employing benzyl ((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one and 4-cyclopropyl-1,2,5-oxadiazole-3-carboxylic acid.

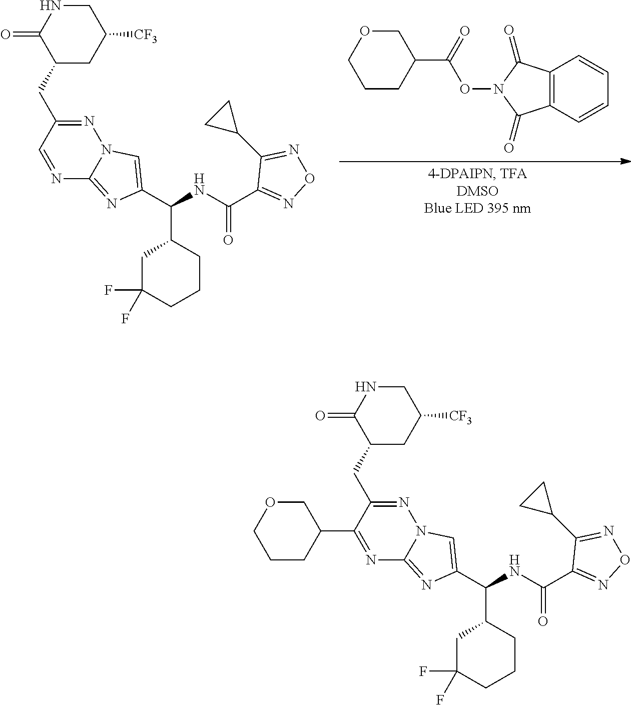 [SMILES: O=C(N[C@H](c1cn2nc(C[C@H]3C[C@@H](C(F)(F)F)CNC3=O)cnc2n1)[C@@H]1CCCC(F)(F)C1)c1nonc1C1CC1 ; O=C(ON1C(=O)c2ccccc2C1=O)C1CCCOC1 ; "4-DPAIPN, TFADMSOBlue LED 395 nm" ; O=C(N[C@H](c1cn2nc(C[C@H]3C[C@@H](C(F)(F)F)CNC3=O)c(C3CCCOC3)nc2n1)[C@@H]1CCCC(F)(F)C1)c1nonc1C1CC1]

4-Cyclopropyl-N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1,2,5-oxadiazole-3-carboxamide was synthesized following the General Procedure 7 with 4-cyclopropyl-N-((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (120 mg, 206 μmol, 1.00 eq), 1,3-dioxoisoindolin-2-yl tetrahydro-2H-pyran-3-carboxylate (113 mg, 412 μmol, 2.00 eq) and was purified by prep-HPLC (column: Phenomenex luna C18 150×25 mm, 10 μm; mobile phase: [water (FA)—acetonitrile]; gradient: 54%-84% B over 6 min) to afford 4-Cyclopropyl-N-((1S)—((R)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1,2,5-oxadiazole-3-carboxamide (80.0 mg, 120 μmol, 58.2% yield) as a yellow solid. LCMS [M+H]⁺=667.3 m/z.

Compound 209 was isolated as the first eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 35%, isocratic elution mode); (33.62 mg, 48.8 μmol, 40.7% yield) and was obtained as a white solid. LCMS [M+H]⁺=667.4 m/z.

Compound 210 was isolated as the second eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 35%, isocratic elution mode); (35.84 mg, 53.12 μmol, 44.26% yield) and was obtained as a light-yellow solid. LCMS [M+H]⁺=667.4 m/z.

### Example 104: Preparation of N-((1S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide (Compound 127)

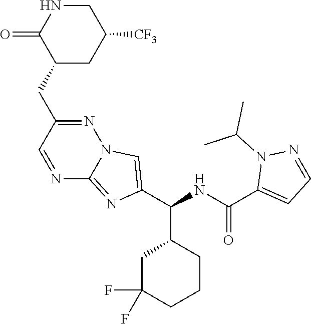 [SMILES: CC(C)n1nccc1C(=O)N[C@H](c1cn2nc(C[C@H]3C[C@@H](C(F)(F)F)CNC3=O)cnc2n1)[C@@H]1CCCC(F)(F)C1]

N-((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl ((S)-3-bromo-1-((R)-3,3-difluorocyclohexyl)-2-oxopropyl)carbamate, General Procedure 5, employing benzyl ((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one and 1-isopropyl-1H-pyrazole-5-carboxylic acid.

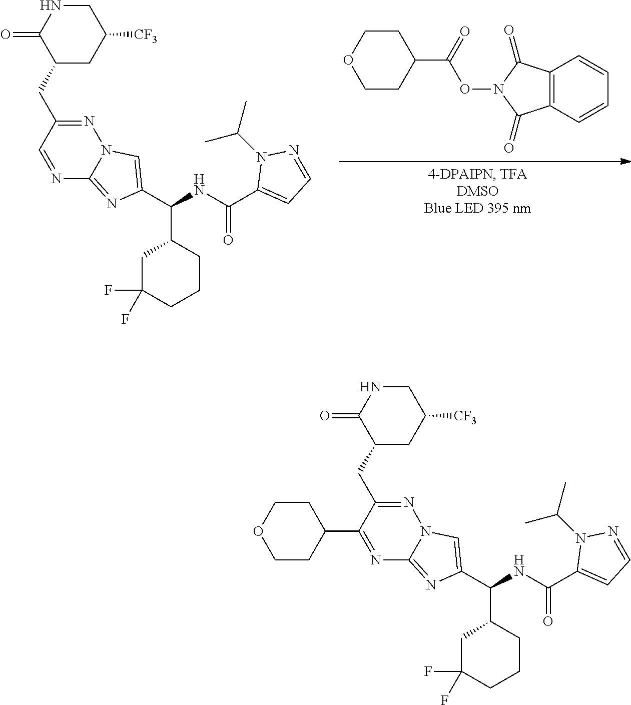 [SMILES: CC(C)n1nccc1C(=O)N[C@H](c1cn2nc(C[C@H]3C[C@@H](C(F)(F)F)CNC3=O)cnc2n1)[C@@H]1CCCC(F)(F)C1 ; O=C(ON1C(=O)c2ccccc2C1=O)C1CCOCC1 ; "4-DPAIPN, TFADMSOBlue LED 395 nm" ; CC(C)n1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]

N-((1S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide was synthesized following the General Procedure 7 with N-((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 120 μmol, 1.00 eq), 1,3-dioxoisoindolin-2-yl tetrahydro-2H-pyran-4-carboxylate (66.1 mg, 240 μmol, 2.00 eq) and was purified by prep-HPLC (basic condition, column: (s, s) WHELK-O1 (250 mm×30 mm, 10 μm); mobile phase: [CO₂-EtOH (0.1% NH₃H₂O)]; B %: 45%, isocratic elution mode) to afford N-((1S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide, Compound 127 (26.67 mg, 39.5 μmol, 32.8% yield) as a yellow solid. LCMS [M+H]⁺=667.4 m/z.

### Example 105: Preparation of N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-isopropyl-1H-pyrazole-5-carboxamide (Compounds 132 and 133)

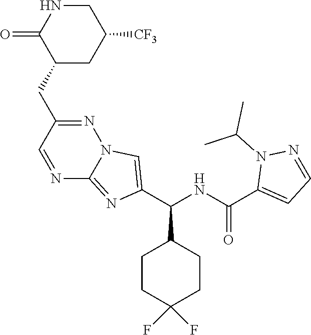 [SMILES: CC(C)n1nccc1C(=O)N[C@H](c1cn2nc(C[C@H]3C[C@@H](C(F)(F)F)CNC3=O)cnc2n1)C1CCC(F)(F)CC1]

N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 5, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one and 1-isopropyl-1H-pyrazole-5-carboxylic acid.

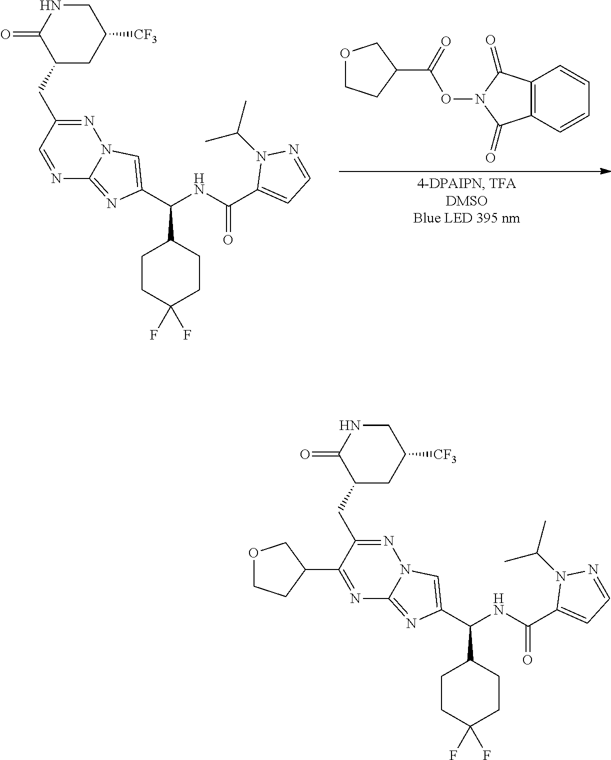 [SMILES: CC(C)n1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCOC1 ; "4-DPAIPN, TFADMSOBlue LED 395 nm" ; CC(C)n1nccc1C(=O)N[C@H](c1cn2nc(C[C@H]3C[C@@H](C(F)(F)F)CNC3=O)c(C3CCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-isopropyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (162 mg, 278 μmol, 1.00 eq), 1,3-dioxoisoindolin-2-yl tetrahydrofuran-3-carboxylate (145 mg, 556 μmol, 2.00 eq) and was purified by prep-HPLC (column: Phenomenex luna C18 150×25 mm, 10 μm; mobile phase: [water (FA)—acetonitrile]; gradient: 47%-77% B over 9 min) to afford N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-isopropyl-1H-pyrazole-5-carboxamide (80.0 mg) as a white solid. LCMS [M+H]⁺=653.4 m/z.

Compound 132 was isolated as the first eluting, single stereoisomer by chiral SFC purification (column: Daicel Chiralcel OD-H (250 mm×30 mm, 5 μm); mobile phase: [CO₂-EtOH]; B %: 35%, isocratic elution mode); (34.18 mg, 50.69 μmol, 41.3% yield) and was obtained as a white solid. LCMS [M+H]⁺=653.4 m/z.

Compound 133 was isolated as the second eluting, single stereoisomer by chiral SFC purification (column: Daicel Chiralcel OD-H (250 mm×30 mm, 5 μm); mobile phase: [CO₂-EtOH]; B %: 35%, isocratic elution mode); (34.62 mg, 52.89 μmol, 43.15% yield) and was obtained as a white solid. LCMS [M+H]⁺=653.4 m/z.

### Example 106: Preparation of 4-cyclopropyl-N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1,2,5-oxadiazole-3-carboxamide (Compounds 134 and 135)

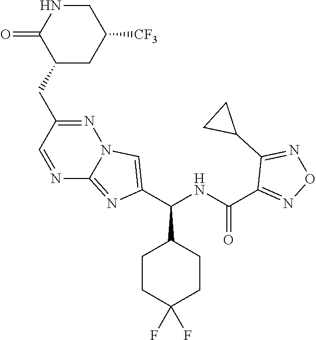 [SMILES: O=C(N[C@H](c1cn2nc(C[C@H]3C[C@@H](C(F)(F)F)CNC3=O)cnc2n1)C1CCC(F)(F)CC1)c1nonc1C1CC1]

4-Cyclopropyl-N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 5, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one and 4-cyclopropyl-1,2,5-oxadiazole-3-carboxylic acid.

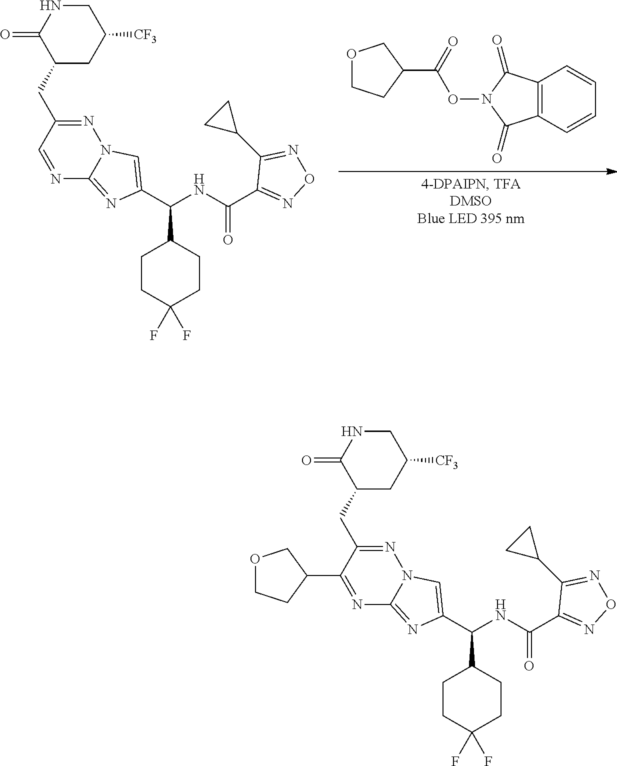 [SMILES: O=C(N[C@H](c1cn2nc(C[C@H]3C[C@@H](C(F)(F)F)CNC3=O)cnc2n1)C1CCC(F)(F)CC1)c1nonc1C1CC1 ; O=C(ON1C(=O)c2ccccc2C1=O)C1CCOC1 ; "4-DPAIPN, TFADMSOBlue LED 395 nm" ; O=C(N[C@H](c1cn2nc(C[C@H]3C[C@@H](C(F)(F)F)CNC3=O)c(C3CCOC3)nc2n1)C1CCC(F)(F)CC1)c1nonc1C1CC1]

4-Cyclopropyl-N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1,2,5-oxadiazole-3-carboxamide was synthesized following the General Procedure 7 with 4-cyclopropyl-N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (158 mg, 271 μmol, 1.00 eq), 1,3-dioxoisoindolin-2-yl tetrahydrofuran-3-carboxylate (70.8 mg, 271 μmol, 1.00 eq) and was purified by prep-HPLC (column: Phenomenex luna C18 150×25 mm, 10 μm; mobile phase: [water (FA)—acetonitrile]; gradient: 44%-74% B over 9 min) to afford 4-cyclopropyl-N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1,2,5-oxadiazole-3-carboxamide (70.0 mg) as a white solid. LCMS [M+H]⁺=653.3 m/z.

Compound 134 was isolated as the first eluting, single stereoisomer by chiral SFC purification (column: Daicel Chiralcel OD-H (250 mm×30 mm, 5 μm); mobile phase: [CO₂-EtOH]; B %: 30%, isocratic elution mode); (33.15 mg, 49.9 μmol, 46.5% yield) and was obtained as a white solid. LCMS [M+H]⁺=653.4 m/z.

Compound 135 was isolated as the second eluting, single stereoisomer by chiral SFC purification (column: Daicel Chiralcel OD-H (250 mm×30 mm, 5 μm); mobile phase: [CO₂-EtOH]; B %: 30%, isocratic elution mode); (35.21 mg, 52.9 μmol, 49.3% yield) and was obtained as a white solid. LCMS [M+H]⁺=653.4 m/z.

### Example 107: Preparation of N-((1S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-ethyl-1H-pyrazole-5-carboxamide (Compound 137)

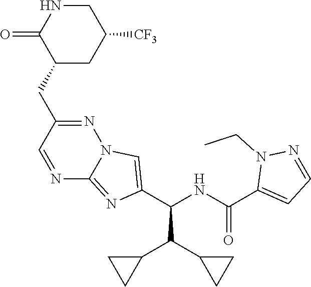 [SMILES: CCn1nccc1C(=O)N[C@H](c1cn2nc(C[C@H]3C[C@@H](C(F)(F)F)CNC3=O)cnc2n1)C(C1CC1)C1CC1]

N-((1S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide was synthesized according to General Procedure 3, employing (3R,5R)-3-((3-amino-1,2,4-triazin-6-yl)methyl)-5-(trifluoromethyl)piperidin-2-one and benzyl (S)-(4-bromo-1,1-dicyclopropyl-3-oxobutan-2-yl)carbamate, General Procedure 4, employing benzyl ((1S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)carbamate, and General Procedure 6, employing (3R,5R)-3-((6-((S)-1-amino-2,2-dicyclopropylethyl)imidazo[1,2-b][1,2,4]triazin-2-yl)methyl)-5-(trifluoromethyl)piperidin-2-one and 1-ethyl-1H-pyrazole-5-carboxylic acid.

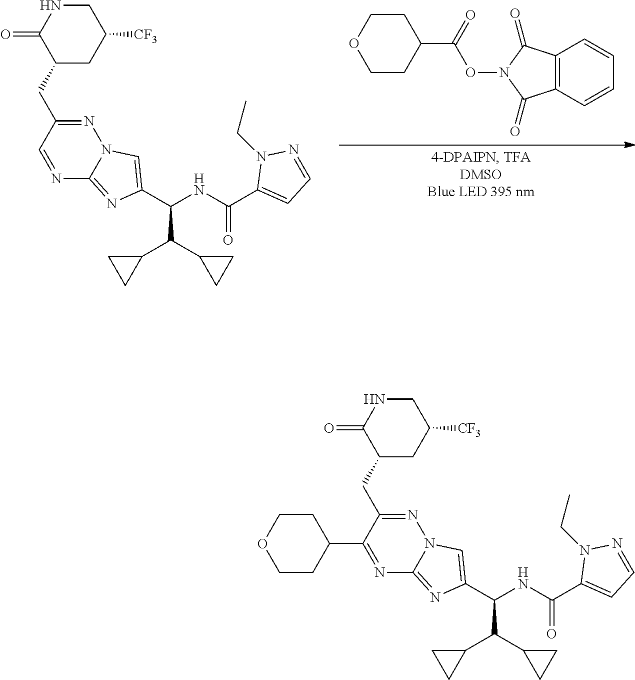 [SMILES: CCn1nccc1C(=O)N[C@H](c1cn2nc(C[C@H]3C[C@@H](C(F)(F)F)CNC3=O)cnc2n1)C(C1CC1)C1CC1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOCC3)nc2n1)C(C1CC1)C1CC1]

N-((1S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-ethyl-1H-pyrazole-5-carboxamide was synthesized following the General Procedure 7 with N-((1S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (120 mg, 220 μmol, 1.00 eq), 1,3-dioxoisoindolin-2-yl tetrahydro-2H-pyran-4-carboxylate (121 mg, 440 μmol, 2.00 eq) and was purified by prep-HPLC (FA condition, column: Phenomenex luna C 18 150×25 mm, 10 μm; mobile phase: [water (FA)—acetonitrile]; gradient: 43%-73% B over 10 min) to afford N-((1S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-ethyl-1H-pyrazole-5-carboxamide, Compound 137 (45.02 mg, 69.1 μmol, 31.3% yield) as a yellow solid. LCMS [M+H]⁺=629.3 m/z.

### Example 108: Preparation of 1-ethyl-N-((1S)-((1r,4S)-4-methylcyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1H-pyrazole-5-carboxamide (Compounds 138 and 139)

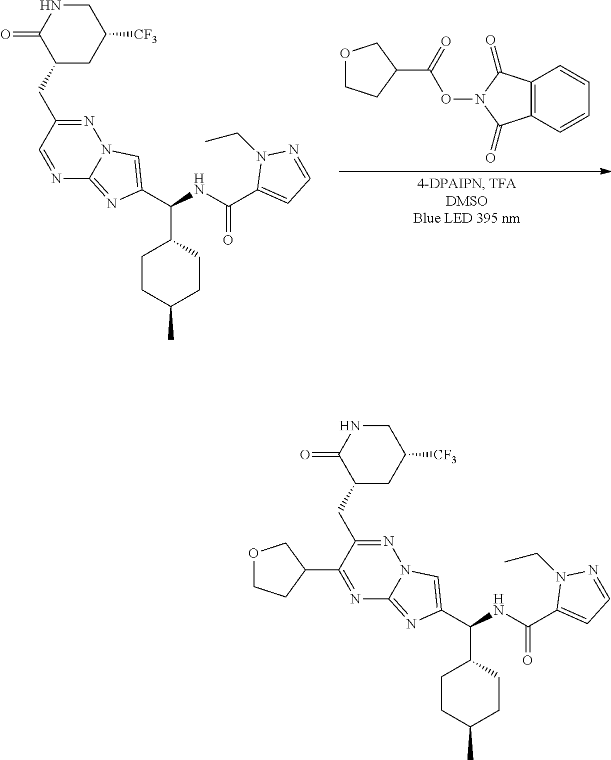 [SMILES: CCn1nccc1C(=O)N[C@H](c1cn2nc(C[C@H]3C[C@@H](C(F)(F)F)CNC3=O)cnc2n1)[C@H]1CC[C@H](C)CC1 ; O=C(ON1C(=O)c2ccccc2C1=O)C1CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC3)nc2n1)[C@H]1CC[C@H](C)CC1]

1-Ethyl-N-((1S)-((1r,4S)-4-methylcyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1H-pyrazole-5-carboxamide was synthesized following the General Procedure 7 with 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (160 mg, 292 mol, 1.00 eq), 1,3-dioxoisoindolin-2-yl tetrahydrofuran-3-carboxylate (153 mg, 585 μmol, 2.00 eq) and was purified by prep-HPLC (column: Phenomenex luna C18 150×25 mm, 10 μm; mobile phase: [water (FA)—acetonitrile]; gradient: 46%-76% B over 9 min) to afford 1-ethyl-N-((1S)-((1r,4S)-4-methylcyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1H-pyrazole-5-carboxamide (120 mg, 194 μmol, 66.5% yield) as a yellow solid. LCMS [M+H]⁺=617.3 m/z.

Compound 138 was isolated as the first eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 40%, isocratic elution mode); (30.9 mg, 49.4 μmol, 25.4% yield) and was obtained as a yellow solid. LCMS [M+H]⁺=617.3 m/z.

Compound 139 was isolated as the first eluting, single stereoisomer by chiral SFC purification (column: Daicel Chiralcel OD (250 mm×30 mm, 10 μm); mobile phase: [CO₂-i-PrOH (0.1% NH₃H₂O)]; B %: 40%, isocratic elution mode); (35.9 mg, 57.7 μmol, 29.6% yield) and was obtained as a yellow solid. LCMS [M+H]⁺=617.3 m/z.

### Example 109: Preparation of N-((1S)-(4,4-difluorocyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 140)

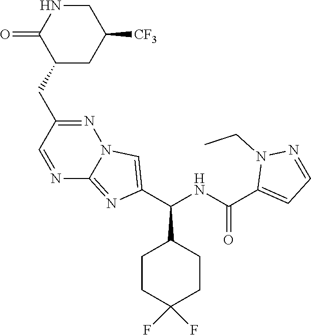 [SMILES: CCn1nccc1C(=O)N[C@H](c1cn2nc(C[C@H]3C[C@H](C(F)(F)F)CNC3=O)cnc2n1)C1CCC(F)(F)CC1]

N-((1S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized according to General Procedure 3, employing (3R,5S)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 5, employing benzyl ((1S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate, and General Procedure 6, employing (3R,5S)-3-((6-((S)-amino(4,4-difluorocyclohexyl)methyl)imidazo[1,2-b][1,2,4]triazin-2-yl)methyl)-5-(trifluoromethyl)piperidin-2-one and 1-ethyl-1H-pyrazole-5-carboxylic acid.

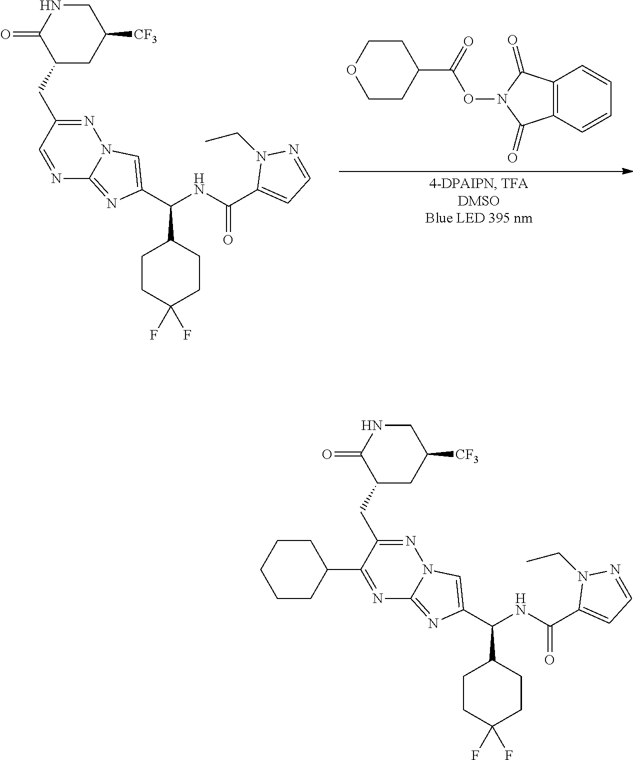 [SMILES: CCn1nccc1C(=O)N[C@H](c1cn2nc(C[C@H]3C[C@H](C(F)(F)F)CNC3=O)cnc2n1)C1CCC(F)(F)CC1 ; O=C(ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@H](C(F)(F)F)CNC3=O)c(C3CCCCC3)nc2n1)C1CCC(F)(F)CC1]

N-((1S)-(4,4-difluorocyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1,3-dioxoisoindolin-2-yl tetrahydro-2H-pyran-4-carboxylate (116 mg, 422 μmol, 2.00 eq) and was purified by prep-HPLC (column: Phenomenex luna C18 150×25 mm, 10 μm; mobile phase: [water (FA)—acetonitrile]; gradient: 35%-65% B over 9 min) to afford N-((1S)-(4,4-difluorocyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide, Compound 140 (33.8 mg, 51.5 μmol, 24.4% yield) as a yellow solid. LCMS [M+H]⁺=653.4 m/z.

### Example 110: Preparation of N-((1S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide (Compound 141)

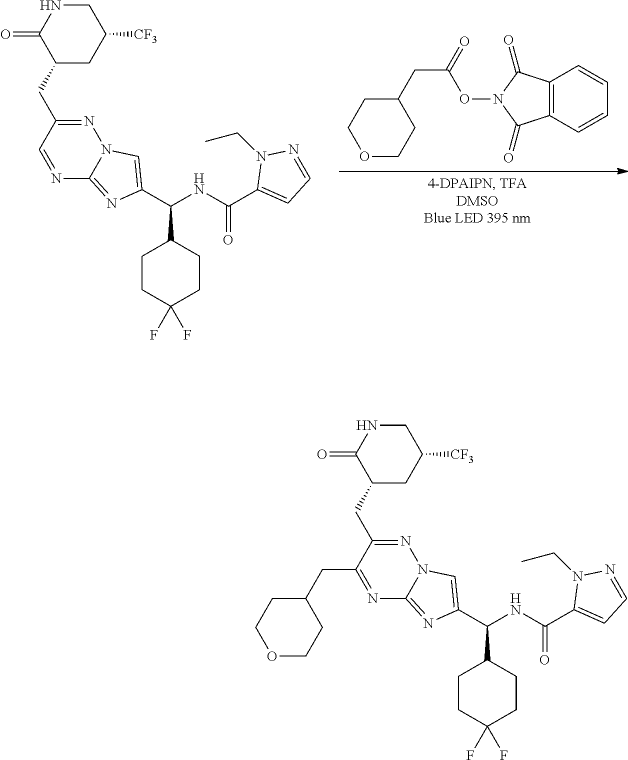 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCOCC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCOCC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq), 1,3-dioxoisoindolin-2-yl 2-(tetrahydro-2H-pyran-4-yl)acetate (101 mg, 351 μmol, 2.00 eq) and was purified by prep-HPLC (column: Phenomenex luna C18 150×25 mm, 10 μm; mobile phase: [water (FA)—acetonitrile]; gradient: 42%-62% B over 10 min) to afford N-((1S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide, Compound 141 (62.73 mg, 91.5 μmol, 52.0% yield) as a yellow solid. LCMS [M+H]⁺=667.4 m/z.

### Example 111: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(2,2-difluorocyclopropyl)isoxazole-4-carboxamide (Compounds 144 and 145)

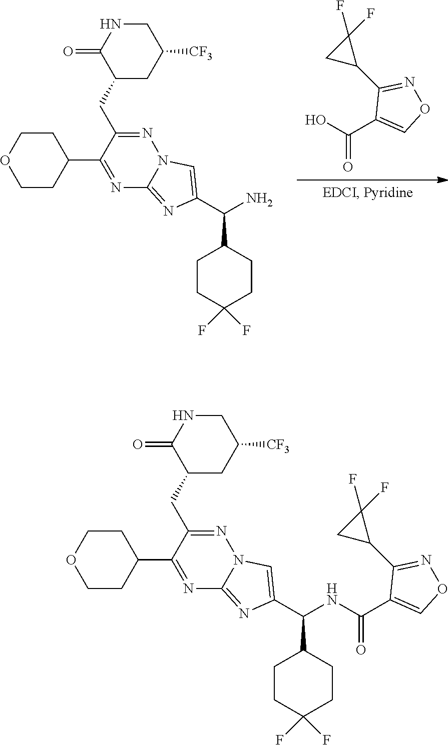

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(2,2-difluorocyclopropyl)isoxazole-4-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (80.0 mg, 150 μmol, 1.00 eq), 3-(2,2-difluorocyclopropyl)isoxazole-4-carboxylic acid (42.7 mg, 226 μmol, 1.50 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(2,2-difluorocyclopropyl)isoxazole-4-carboxamide (70.0 mg, 99.7 μmol, 66.1% yield) as a white solid. LCMS [M+H]⁺=702.2 m/z.

Compound 144 was isolated as the first eluting, single stereoisomer by chiral SFC purification (column: Daicel Chiralpak AD (250 mm×30 mm, 10 μm); mobile phase: [CO₂-i-PrOH (0.1% NH₃H₂O)]; B %: 30%, isocratic elution mode); (35.43 mg, 48.7 μmol, 48.8% yield) and was obtained as a white solid. LCMS [M+H]⁺=702.3 m/z.

Compound 145 was isolated as the first eluting, single stereoisomer by chiral SFC purification (column: Daicel Chiralpak AD (250 mm×30 mm, 10 μm); mobile phase: [CO₂-i-PrOH (0.1% NH₃H₂O)]; B %: 30%, isocratic elution mode); (33.58 mg, 46.5 μmol, 46.6% yield) and was obtained as a white solid. LCMS [M+H]⁺=702.3 m/z.

### Example 112: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-ethylisoxazole-4-carboxamide (Compound 146)

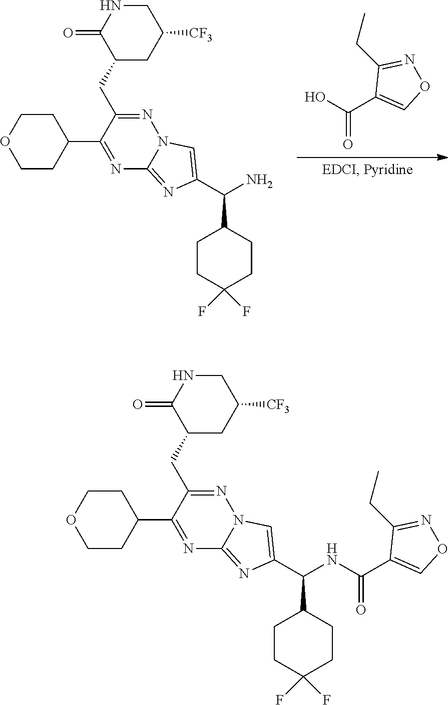

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-ethylisoxazole-4-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 3-ethylisoxazole-4-carboxylic acid (16.0 mg, 113 μmol, 1.20 eq) and was purified by prep-HPLC (column: Phenomenex luna C18 150×25 mm, 10 μm; mobile phase: [water (FA)—acetonitrile]; gradient: 36%-66% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-ethylisoxazole-4-carboxamide, Compound 146 (41.4 mg, 61.9 μmol, 65.7% yield) as a light yellow solid. LCMS [M+H]⁺=654.5 m/z.

### Example 113: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (Compound 150)

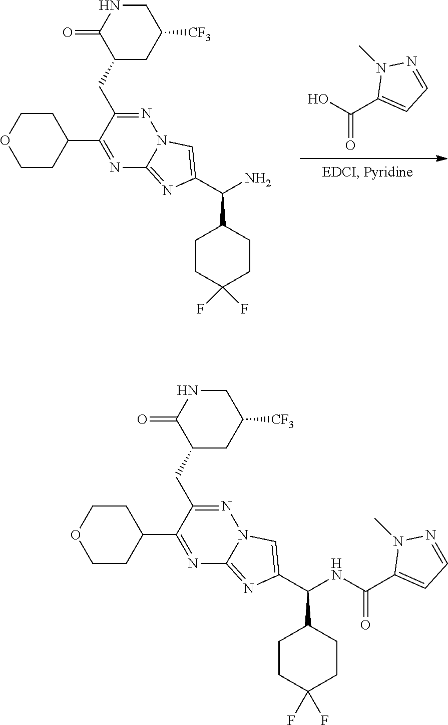

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 1-methyl-1H-pyrazole-5-carboxylic acid (14.2 mg, 113 μmol, 1.20 eq) and was purified by prep-HPLC (column: Phenomenex luna C18 150×25 mm, 10 μm; mobile phase: [water (FA)—acetonitrile], gradient: 32%-62% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide, Compound 150 (44.2 mg, 68.8 μmol, 73.0% yield) as a light yellow solid. LCMS [M+H]⁺=639.5 m/z.

### Example 114: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide (Compound 147)

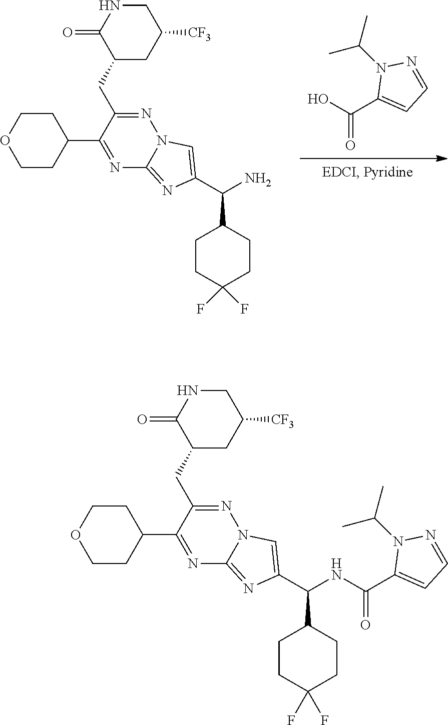

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 1-isopropyl-1H-pyrazole-5-carboxylic acid (17.4 mg, 113 μmol, 1.50 eq) and was purified by prep-HPLC (column: Waters Xbridge 150×25 mm, 5 μm; mobile phase: [water (ammonia hydroxide)—acetonitrile]; gradient: 35%-65% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-isopropyl-1H-pyrazole-5-carboxamide, Compound 147 (27.12 mg, 40.3 μmol, 53.5% yield) as a white solid. LCMS [M+H]⁺=667.3 m/z.

### Example 115: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 148)

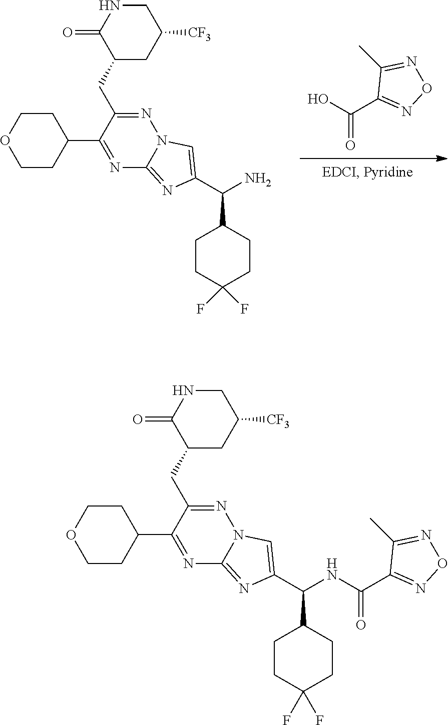

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 4-methyl-1,2,5-oxadiazole-3-carboxylic acid (11.6 mg, 90.5 μmol, 1.20 eq) and was purified by prep-HPLC (column: Phenomenex luna C18 150×25 mm, 10 μm; mobile phase: [water (FA)—acetonitrile]; gradient: 45%-75% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide, Compound 148 (29.1 mg, 45.0 μmol, 59.7% yield) as a white solid. LCMS [M+H]⁺=641.4 m/z.

### Example 116: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (Compound 149)

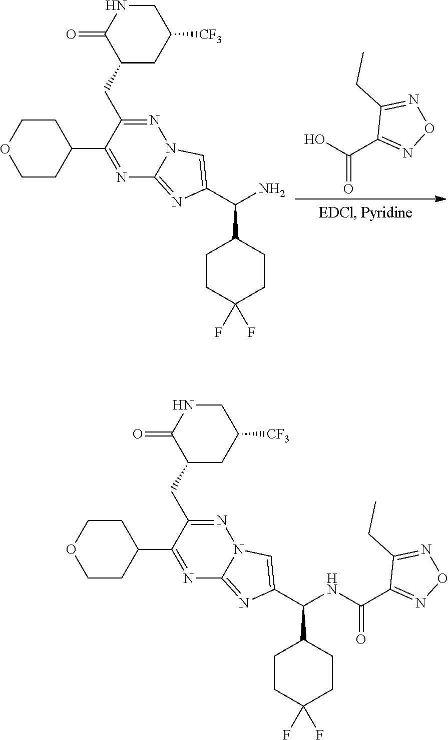

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 4-ethyl-1,2,5-oxadiazole-3-carboxylic acid (12.9 mg, 90.5 μmol, 1.20 eq) and was purified by prep-HPLC (column: Phenomenex luna C18 150×25 mm, 10 μm; mobile phase: [water (FA)—acetonitrile]; gradient: 46%-66% B over 10 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide, Compound 149 (32.1 mg, 48.8 μmol, 64.7% yield) as a white solid. LCMS [M+H]⁺=655.5 m/z.

### Example 117: Preparation of N-((1S)-(3-(8-oxabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 153)

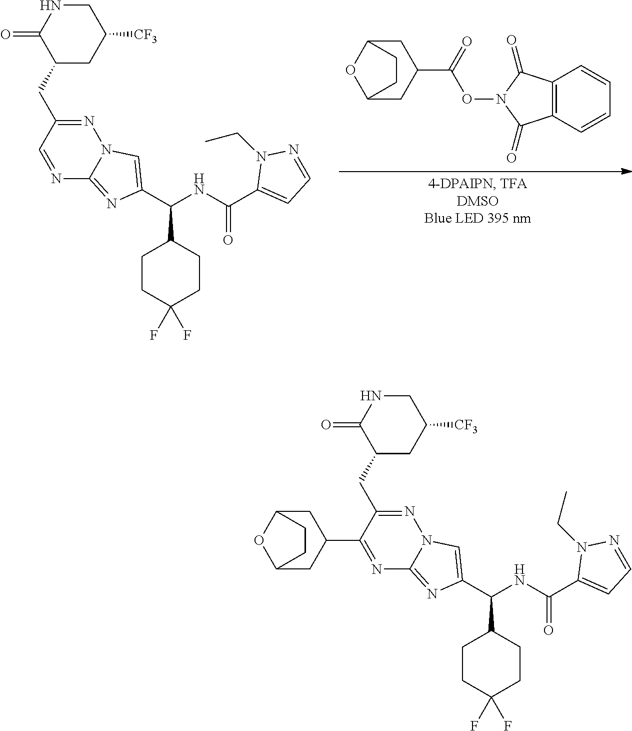 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CC2CCC(C1)O2 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CC4CCC(C3)O4)nc2n1)C1CCC(F)(F)CC1]

N-((1S)-(3-(8-oxabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq), 1,3-dioxoisoindolin-2-yl 8-oxabicyclo[3.2.1]octane-3-carboxylate (106 mg, 351 μmol, 2.00 eq) and was purified by prep-HPLC (column: Phenomenex luna C18 150×25 mm, 10 μm; mobile phase: [water (FA)—acetonitrile]; gradient: 45%-75% B over 10 min) to afford N-((1S)-(3-(8-oxabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide, Compound 153 (51.12 mg, 75.32 μmol, 42.82% yield) as a white solid. LCMS [M+H]⁺=679.4 m/z.

### Example 118: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (Compound 154)

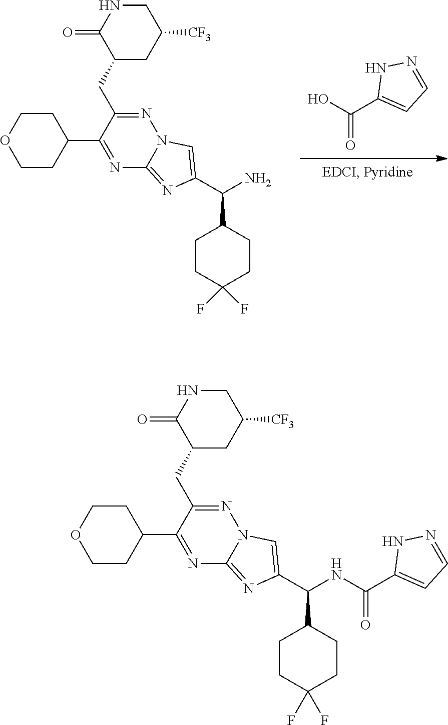

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 1H-pyrazole-5-carboxylic acid (31.7 mg, 283 μmol, 3.00 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide, Compound 154 (35.8 mg, 52.7 μmol, 55.9% yield) as a white solid. LCMS [M+H]⁺=625.3 m/z.

### Example 119: Preparation of 1-ethyl-N-((1S)-((1r,4S)-4-methylcyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (Compound 155)

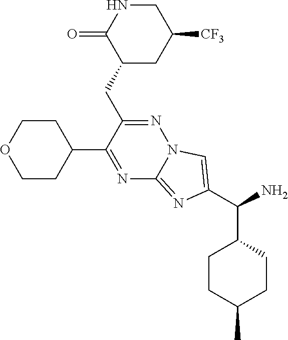 [SMILES: C[C@H]1CC[C@H]([C@H](N)c2cn3nc(C[C@H]4C[C@H](C(F)(F)F)CNC4=O)c(C4CCOCC4)nc3n2)CC1]

(3R,5S)-3-((6-((S)-amino((1r,4S)-4-methylcyclohexyl)methyl)-3-(tetrahydro-2H-pyran-4-yl)imidazo[1,2-b][1,2,4]triazin-2-yl)methyl)-5-(trifluoromethyl)piperidin-2-one was synthesized according to General Procedure 3, employing (3R,5S)-3-((3-amino-1,2,4-triazin-6-yl)methyl)-5-(trifluoromethyl)piperidin-2-one and benzyl ((S)-3-bromo-1-((1r,4S)-4-methylcyclohexyl)-2-oxopropyl)carbamate, General Procedure 7, employing benzyl ((1S)-((1r,4S)-4-methylcyclohexyl)(2-(((3R,5S)-2-oxo-5-(trifluoromethyl)piperidin-3-yl)methyl)imidazo[1,2-b][1,2,4]triazin-6-yl)methyl)carbamate and 1,3-dioxoisoindolin-2-yl tetrahydro-2H-pyran-4-carboxylate, and General Procedure 5, employing benzyl ((1S)-((1r,4S)-4-methylcyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate.

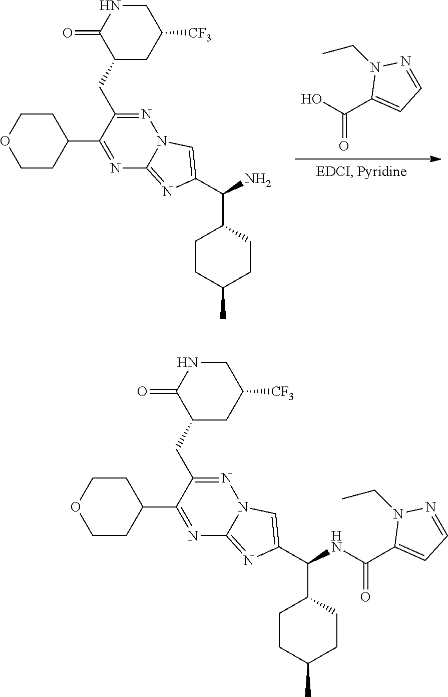

1-Ethyl-N-((1S)-((1r,4S)-4-methylcyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide was synthesized following the General Procedure 6 with (3R,5S)-3-((6-((S)-amino((1r,4S)-4-methylcyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 91.7 mol, 1.00 eq, HCl salt), 1-ethyl-1H-pyrazole-5-carboxylic acid (19.3 mg, 137 μmol, 1.50 eq) and was purified by prep-HPLC (column: Phenomenex luna C18 150×25 mm, 10 μm; mobile phase: [water (FA)—acetonitrile]; gradient: 49%-69% B over 10 min) to afford 1-ethyl-N-((1S)-((1r,4S)-4-methylcyclohexyl)(2-(((3R,5S)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide, Compound 155 (14.5 mg, 22.5 μmol, 24.5% yield) as a white solid. LCMS [M+H]⁺=631.3 m/z.

### Example 120: Preparation of N-((1S)-(4,4-difluorocyclohexyl)(3-(2,2-dimethyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 163 and 176)

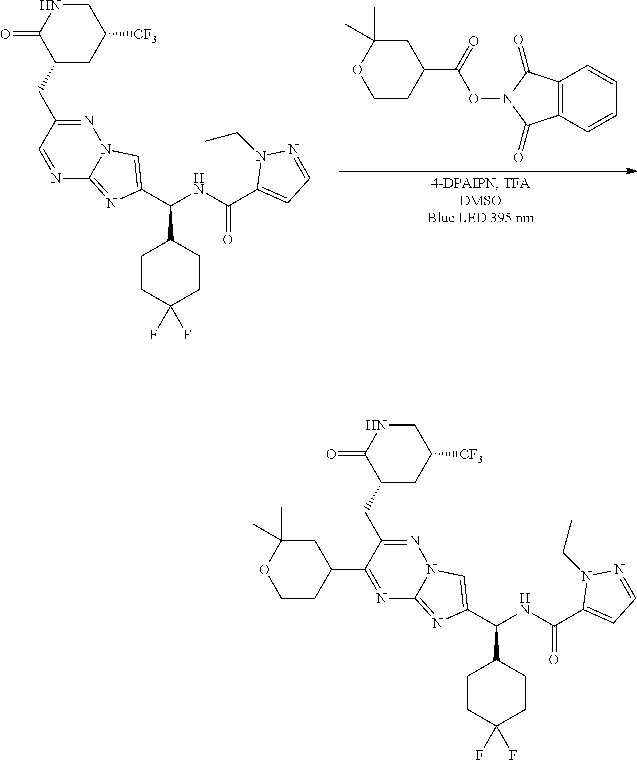 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC1(C)CC(C(=O)ON2C(=O)c3ccccc3C2=O)CCO1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC(C)(C)C3)nc2n1)C1CCC(F)(F)CC1]

N-((1S)-(4,4-difluorocyclohexyl)(3-(2,2-dimethyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (140 mg, 246 μmol, 1.00 eq), 1,3-dioxoisoindolin-2-yl 2,2-dimethyltetrahydro-2H-pyran-4-carboxylate (149 mg, 492 μmol, 2.00 eq) and was purified by prep-HPLC (column: Phenomenex luna C18 150×25 mm, 10 μm; mobile phase: [water (FA)—acetonitrile]; gradient: 40%-70% B over 9 min to afford N-((1S)-(4.4-difluorocyclohexyl)(3-(2,2-dimethyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 176.29 μmol, 71.59% yield) as a yellow solid. LCMS [M+H]⁺=681.3 m/z.

Compound 163 was isolated as the first eluting, single stereoisomer by chiral SFC purification (column: Daicel Chiralpak IK (250 mm×30 mm, 10 μm); mobile phase: [CO₂-i-PrOH]; B %: 40%, isocratic elution mode); (50.03 mg, 72.6 μmol, 41.2% yield) and was obtained as a white solid. LCMS [M+H]⁺=681.3 m/z.

Compound 176 was isolated as the second eluting, single stereoisomer by chiral SFC purification (column: Daicel Chiralpak IK (250 mm×30 mm, 10 μm); mobile phase: [CO₂-i-PrOH]; B %: 40%, isocratic elution mode); (36.12 mg, 52.8 μmol, 29.9% yield) and was obtained as a white solid. LCMS [M+H]⁺=681.3 m/z.

### Example 121: Preparation of N-((1S)-(4,4-difluorocyclohexyl)(3-(4-fluorotetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 212)

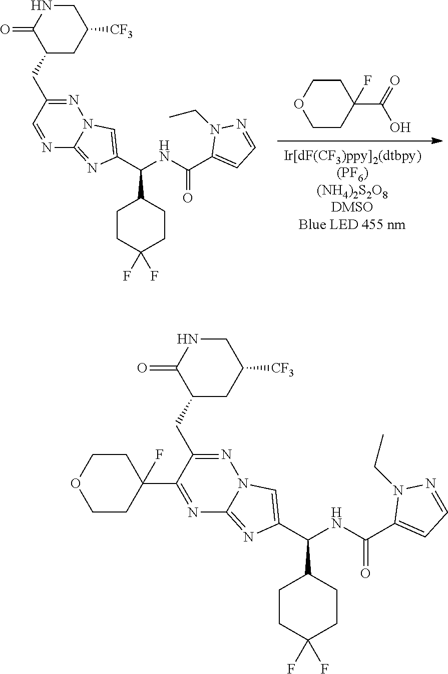

To a solution of N-((1S)-(4,4-difluorocyclohexyl)(2-(((3R-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq) and 4-fluorotetrahydro-2H-pyran-4-carboxylic acid (78.1 mg, 527 μmol, 3.00 eq) in DMSO (3.00 mL) was added Ir[dF(CF₃)ppy]₂(dtbpy)(PF₆) (19.7 mg, 17.5 μmol, 0.10 eq) and (NH₄)₂S₂O₈ (47.9 mg, 703 μmol, 48.0 μL, 4.00 eq). The vial was sealed and placed under nitrogen. The reaction mixture was stirred and irradiated with a 455 nm LED lamp, with cooling fan to keep the reaction temperature at room temperature for 24 h. The reaction mixture was diluted with water and extracted with ethyl acetate, dried over anhydrous sodium sulfate, filtered, and concentrated under reduced pressure to give a residue, which was purified by prep-HPLC (column: Phenomenex luna C18 150×25 mm, 10 μm; mobile phase: [water (FA)—acetonitrile]; gradient: 42%-62% B over 10 min) to afford N-((1S)-(4,4-difluorocyclohexyl)(3-(4-fluorotetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 212 (15.9 mg, 23.0 μmol, 13.1% yield) as a white solid. LCMS [M+H]⁺=671.3 m/z.

### Example 122: Preparation of N-((1S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 164)

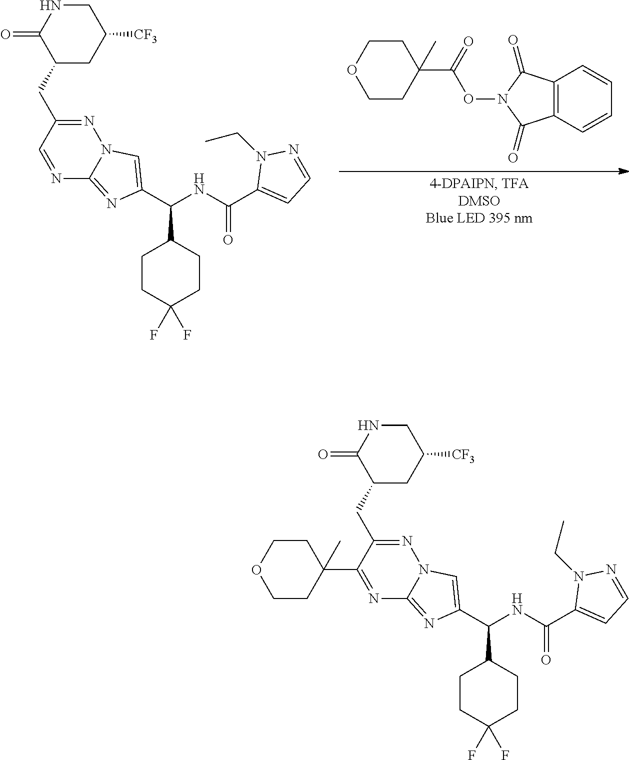 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC1(C(=O)ON2C(=O)c3ccccc3C2=O)CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3(C)CCOCC3)nc2n1)C1CCC(F)(F)CC1]

N-((1S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 106 μmol, 1.00 eq), 1,3-dioxoisoindolin-2-yl 4-methyltetrahydro-2H-pyran-4-carboxylate (61.1 mg, 211 μmol, 2.00 eq) and was purified by prep-HPLC (column: Phenomenex luna C18 150×25 mm, 10 μm; mobile phase: [water (FA)—acetonitrile]; gradient: 42%-66% B over 8 min) to afford N-((1S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1, 2,4]triazin-6-yl)methyl)-1-ethyl-1H-pyrazole-5-carboxamide, Compound 164 (26.94 mg, 39.7 μmol, 37.6% yield) as a light yellow solid. LCMS [M+H]⁺=667.2 m/z.

### Example 123: Preparation of N-((1S)-(3-((1,4-dioxan-2-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 173)

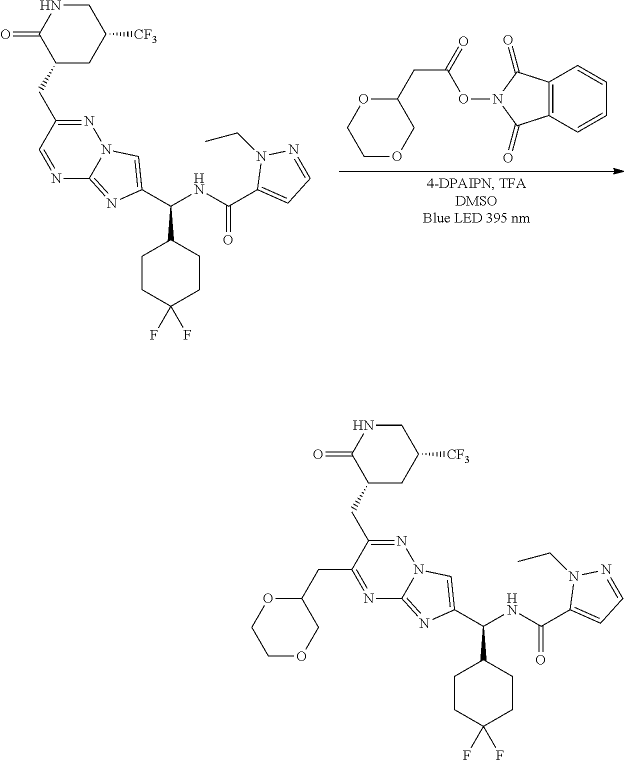 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1COCCO1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3COCCO3)nc2n1)C1CCC(F)(F)CC1]

N-((1S)-(3-((1,4-dioxan-2-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 2-(1,4-dioxan-2-yl)acetate (36.8 mg, 126 μmol, 1.20 eq) and was purified by prep-HPLC (column: Waters Xbridge 150×25 mm, 5 μm; mobile phase: [water (NH₄HCO₃) —acetonitrile]; gradient: 31%-51% B over 10 min) to afford N-((1S)-(3-((1,4-dioxan-2-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide, Compound 173 (12.1 mg, 17.3 μmol, 16.4% yield) as a yellow solid. LCMS [M+H]⁺=669.4 m/z.

### Example 124: Preparation of N-((1S)-(4,4-difluorocyclohexyl)(3-(3-methyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 165 and 177)

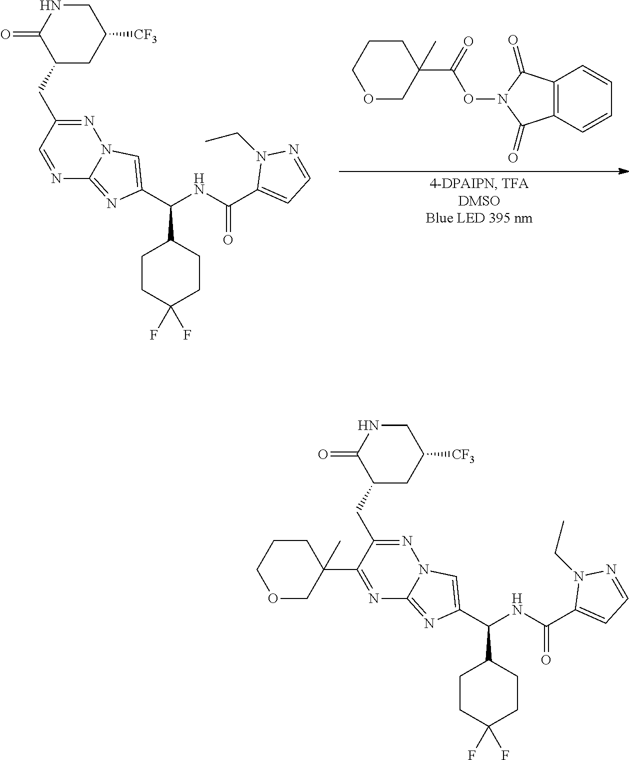 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC1(C(=O)ON2C(=O)c3ccccc3C2=O)CCCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3(C)CCCOC3)nc2n1)C1CCC(F)(F)CC1]

N-((1S)-(4,4-difluorocyclohexyl)(3-(3-methyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (130 mg, 228 μmol, 1.00 eq), 1,3-dioxoisoindolin-2-yl 3-methyltetrahydro-2H-pyran-3-carboxylate (132 mg, 457 μmol, 2.00 eq) and was purified by prep-TLC (SiO₂, dichloromethane:methanol) to afford N-((1S)-(4,4-difluorocyclohexyl)(3-(3-methyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 75.0 μmol, 32.8% yield) as a light yellow solid. LCMS [M+H]⁺=667.3 m/z.

Compound 165 was isolated as the first eluting, single stereoisomer by chiral SFC purification (column: Daicel Chiralpak IG (250 mm×30 mm, 10 μm); mobile phase: [CO₂-i-PrOH]; B %: 50%, isocratic elution mode); (21.84 mg, 29.7 μmol, 39.6% yield) and was obtained as a yellow solid. LCMS [M+H]⁺=667.4 m/z.

Compound 177 was isolated as the second eluting, single stereoisomer by chiral SFC purification (column: Daicel Chiralpak IG (250 mm×30 mm, 10 μm); mobile phase: [CO₂-i-PrOH]; B %: 50%, isocratic elution mode); (22.54 mg, 32.7 μmol, 43.6% yield) and was obtained as a yellow solid. LCMS [M+H]⁺=667.4 n/z.

### Example 125: Preparation of N-((1S)-(4,4-difluorocyclohexyl)(3-(3-methyltetrahydrofuran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 166 and 178)

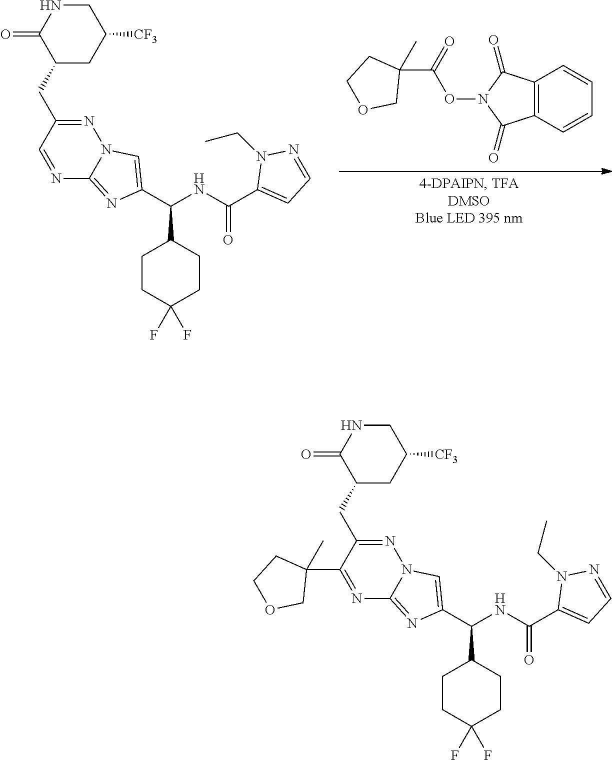 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC1(C(=O)ON2C(=O)c3ccccc3C2=O)CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3(C)CCOC3)nc2n1)C1CCC(F)(F)CC1]

N-((1S)-(4,4-difluorocyclohexyl)(3-(3-methyltetrahydrofuran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1,3-dioxoisoindolin-2-yl 3-methyltetrahydrofuran-3-carboxylate (145 mg, 527 μmol, 2.50 eq) and was purified by prep-HPLC (column: Phenomenex luna C18 150×25 mm, 10 μm; mobile phase: [water (FA)—acetonitrile]; gradient: 41%-71% B over 10 min) to afford N-((1S)-(4,4-difluorocyclohexyl)(3-(3-methyltetrahydrofuran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 98.7 μmol, 46.8% yield) as a white solid. LCMS [M+H]⁺=653.3 m/z.

Compound 166 was isolated as the first eluting, single stereoisomer by chiral SFC purification (column: Daicel Chiralpak IK (250 mm×30 mm, 10 μm); mobile phase: [CO₂-i-PrOH]; B %: 40%, isocratic elution mode); (31.96 mg, 47.8 μmol, 44.6% yield) and was obtained as a white solid. LCMS [M+H]⁺=653.3 m/z.

Compound 178 was isolated as the first eluting, single stereoisomer by chiral SFC purification (column: Daicel Chiralpak IK (250 mm×30 mm, 10 μm); mobile phase: [CO₂-i-PrOH]; B %: 40%, isocratic elution mode); (28.9 mg, 43.3 μmol, 40.4% yield) and was obtained as a white solid. LCMS [M+H]⁺=653.3 m/z.

### Example 126: Preparation of N-((1S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 167)

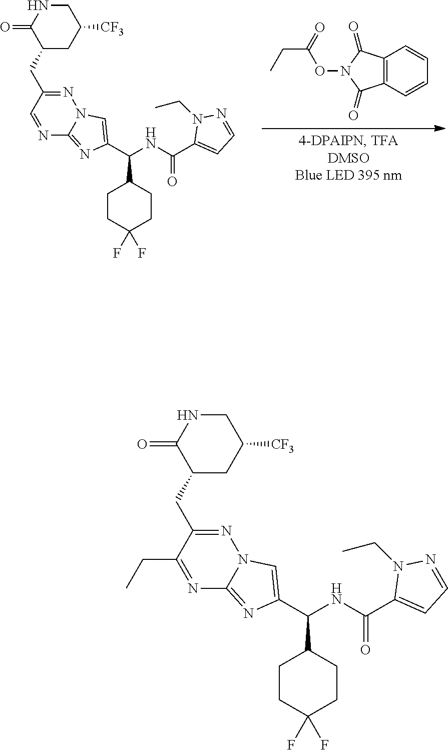 [SMILES: CCn1nccc1C(=O)N[C@H](c1cn2nc(C[C@H]3C[C@@H](C(F)(F)F)CNC3=O)cnc2n1)C1CCC(F)(F)CC1 ; CCC(=O)ON1C(=O)c2ccccc2C1=O ; "4-DPAIPN, TFADMSOBlue LED 395 nm" ; CCc1nc2nc([C@@H](NC(=O)c3ccnn3CC)C3CCC(F)(F)CC3)cn2nc1C[C@H]1C[C@@H](C(F)(F)F)CNC1=O]

N-((1S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl propionate (67.4 mg, 307 μmol, 2.50 eq) and was purified by prep-HPLC (column: Phenomenex luna C18 150×25 mm, 10 μm; mobile phase: [water (FA)—acetonitrile]; gradient: 47%-67% B over 10 min) to afford N-((1S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 167 (32.24 mg, 52.6 μmol, 42.7% yield) as a white solid. LCMS [M+H]⁺=597.4 m/z.

### Example 127: Preparation of N-((1S)-(4,4-difluorocyclohexyl)(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 168)

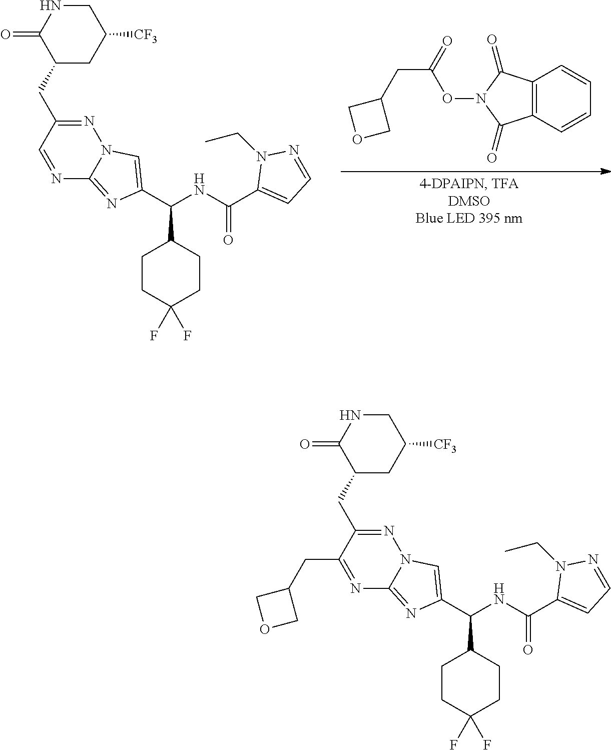 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1COC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3COC3)nc2n1)C1CCC(F)(F)CC1]

N-((1S)-(4,4-difluorocyclohexyl)(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 106 μmol, 1.00 eq), 1,3-dioxoisoindolin-2-yl 2-(oxetan-3-yl)acetate (55.1 mg, 211 μmol, 2.00 eq) and was purified by prep-HPLC (column: Waters Xbridge 150×25 mm, 10 μm; mobile phase: [water (NH₄HCO₃)—acetonitrile]; gradient: 32%-62% B over 10 min) to afford N-((1S)-(4,4-difluorocyclohexyl)(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 168 (20.00 mg, 30.1 μmol, 28.5% yield) as a white solid. LCMS [M+H]⁺=639.3 m/z.

### Example 128: Preparation of N-((1S)-(4,4-difluorocyclohexyl)(2-(((3R,5R)-2-oxo-5-(trifluoromethyl)piperidin-3-yl)methyl)-3-(2-(tetrahydro-2H-pyran-4-yl)propan-2-yl)imidazo[1,2-b][1,2,4]triazin-6-yl)methyl)-1-ethyl-1H-pyrazole-5-carboxamide (Compound 169)

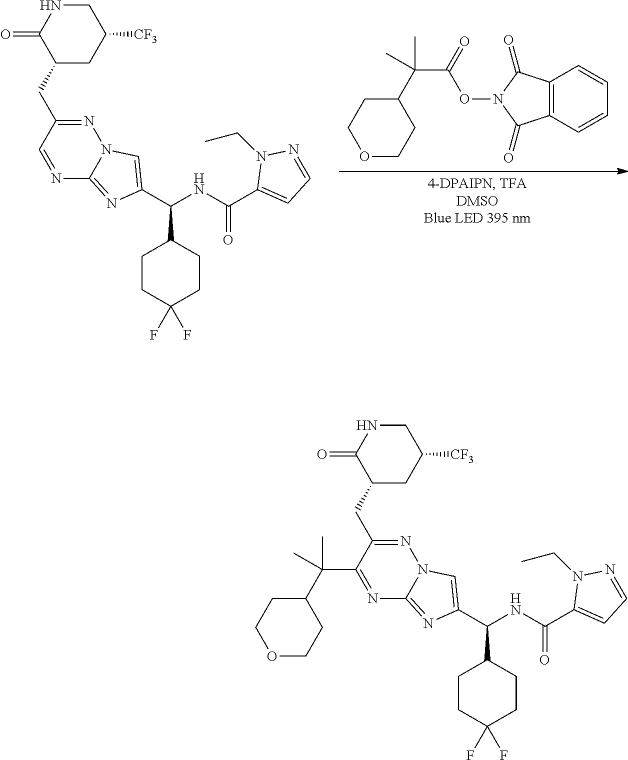 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C(=O)ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)(C)C3CCOCC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(2-(tetrahydro-2H-pyran-4-yl)propan-2-yl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 2-methyl-2-(tetrahydro-2H-pyran-4-yl)propanoate (66.9 mg, 211 μmol, 2.00 eq) and was purified by prep-TLC (SiO₂, petroleum ether:ethyl acetate) to afford N-((1S)-(4,4-difluorocyclohexyl)(2-(((3R,5R)-2-oxo-5-(trifluoromethyl)piperidin-3-yl)methyl)-3-(2-(tetrahydro-2H-pyran-4-yl)propan-2-yl)imidazo[1,2-b][1,2,4]triazin-6-yl)methyl)-1-ethyl-1H-pyrazole-5-carboxamide, Compound 169 (7.11 mg, 8.40 μmol, 7.96% yield) as a yellow solid. LCMS [M+H]⁺=695.4 m/z.

### Example 129: Preparation of N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide (Compound 170)

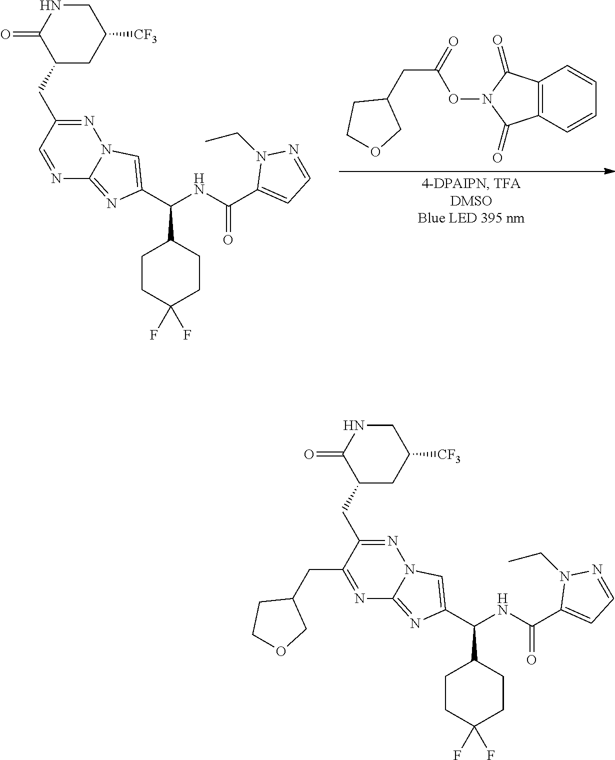 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCOC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydrofuran-3-yl)acetate (67.7 mg, 246 μmol, 2.00 eq) and was purified by prep-HPLC (column: Phenomenex luna C18 150×25 mm, 10 μm; mobile phase: [water (FA)—acetonitrile]; gradient: 40%-60% B over 10 min) to afford N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide, Compound 170 (5.00 mg, 7.34 μmol, 5.96% yield) as a white solid. LCMS [M+H]⁺=653.4 m/z.

### Example 130: Preparation of N-((1S)-(4,4-difluorocyclohexyl)(2-(((3R,5R)-2-oxo-5-(trifluoromethyl)piperidin-3-yl)methyl)-3-((tetrahydrofuran-2-yl)methyl)imidazo[1,2-b][1,2,4]triazin-6-yl)methyl)-1-ethyl-1H-pyrazole-5-carboxamide (Compound 171)

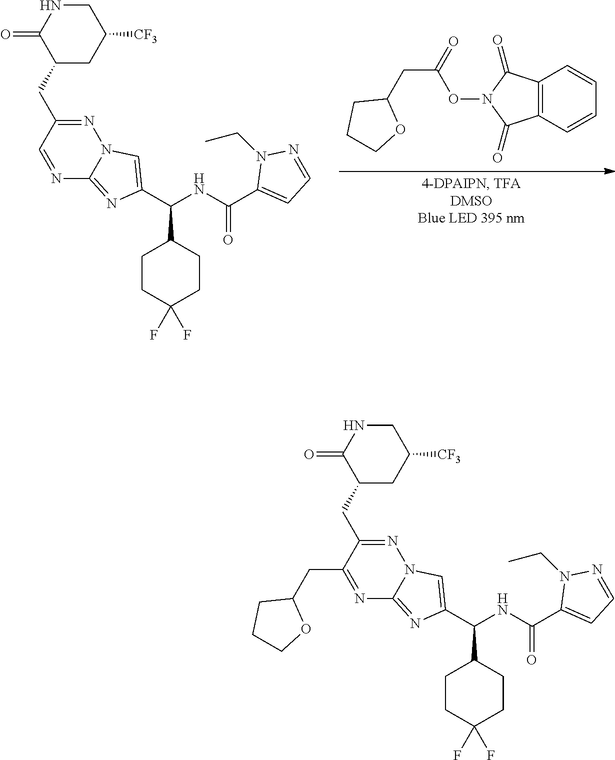 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCCO1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCCO3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydrofuran-2-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydrofuran-2-yl)acetate (67.7 mg, 246 μmol, 2.00 eq) and was purified by prep-HPLC (column: Phenomenex luna C18 150×25 mm, 10 μm; mobile phase: [water (FA)—acetonitrile]; gradient: 41%-71% B over 10 min) to afford N-((1S)-(4,4-difluorocyclohexyl)(2-(((3R,5R)-2-oxo-5-(trifluoromethyl)piperidin-3-yl)methyl)-3-((tetrahydrofuran-2-yl)methyl)imidazo[1,2-b][1,2,4]triazin-6-yl)methyl)-1-ethyl-1H-pyrazole-5-carboxamide, Compound 171 (5.02 mg, 6.94 μmol, 9.63% yield) as a yellow solid. LCMS [M+H]⁺=653.3 m/z.

### Example 131: Preparation of N-((1S)-(4,4-difluorocyclohexyl)(2-(((3R,5R)-2-oxo-5-(trifluoromethyl)piperidin-3-yl)methyl)-3-((tetrahydro-2H-pyran-3-yl)methyl)imidazo[1,2-b][1,2,4]triazin-6-yl)methyl)-1-ethyl-1H-pyrazole-5-carboxamide (Compound 213)

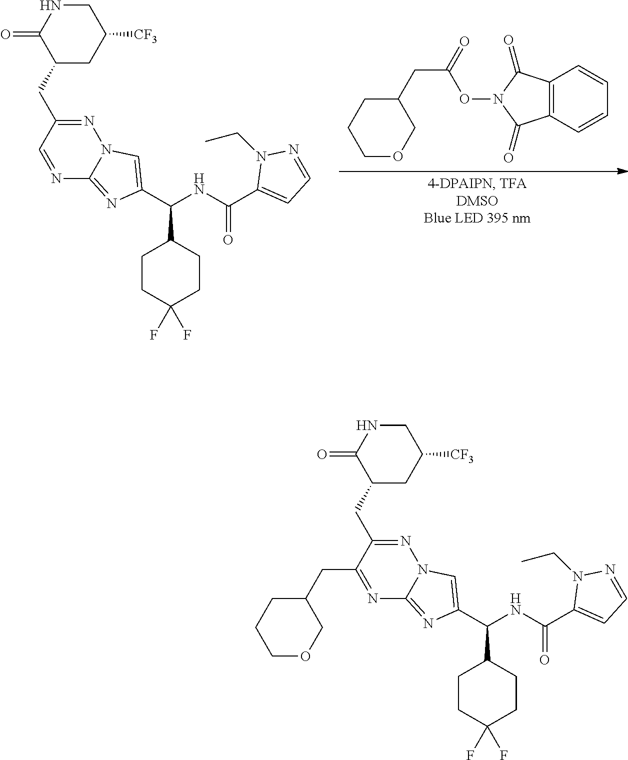 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCCOC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydro-2H-pyran-3-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydro-2H-pyran-3-yl)acetate (39.1 mg, 135 μmol, 1.10 eq) and was purified by prep-HPLC (column: Phenomenex luna C18 150×25 mm, 10 μm; mobile phase: [water (FA)—acetonitrile]; gradient: 42%-62% B over 10 min) to afford N-((1S)-(4,4-difluorocyclohexyl)(2-(((3R,5R)-2-oxo-5-(trifluoromethyl)piperidin-3-yl)methyl)-3-((tetrahydro-2H-pyran-3-yl)methyl)imidazo[1,2-b][1,2,4]triazin-6-yl)methyl)-1-ethyl-1H-pyrazole-5-carboxamide, Compound 213 (18.0 mg, 26.9 μmol, 21.8% yield) as a yellow solid. LCMS [M+H]⁺=667.4 m, z.

### Example 132: Preparation of N-((1S)-(4,4-difluorocyclohexyl)(2-(((3R,5R)-2-oxo-5-(trifluoromethyl)piperidin-3-yl)methyl)-3-((tetrahydro-2H-pyran-2-yl)methyl)imidazo[1,2-b][1,2,4]triazin-6-yl)methyl)-1-ethyl-1H-pyrazole-5-carboxamide (Compound 172)

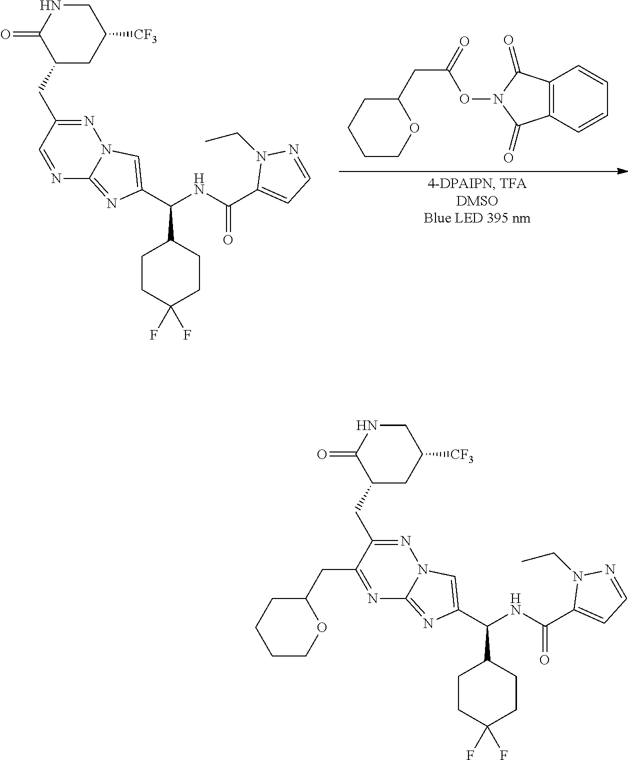 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCCCO1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCCCO3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydro-2H-pyran-2-yl)methyl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydro-2H-pyran-2-yl)acetate (89.0 mg, 307 μmol, 2.50 eq) and was purified by prep-HPLC (column: Phenomenex luna C18 150×25 mm, 10 μm; mobile phase: [water (FA)—acetonitrile]; gradient: 45%-65% B over 10 min) to afford N-((1S)-(4,4-difluorocyclohexyl)(2-(((3R,5R)-2-oxo-5-(trifluoromethyl)piperidin-3-yl)methyl)-3-((tetrahydro-2H-pyran-2-yl)methyl)imidazo[1,2-b][1,2,4]triazin-6-yl)methyl)-1-ethyl-1H-pyrazole-5-carboxamide, Compound 172 (42.56 mg, 63.2 μmol, 51.3% yield) as a white solid. LCMS [M+H]⁺=667.4 m, z.

### Example 133: Preparation of N-((1S)-(4,4-difluorocyclohexyl)(2-(((3R,5R)-2-oxo-5-(trifluoromethyl)piperidin-3-yl)methyl)-3-(1,1,1-trifluoropropan-2-yl)imidazo[1,2-b][1,2,4]triazin-6-yl)methyl)-1-ethyl-1H-pyrazole-5-carboxamide (Compounds 214, 174, and 175)

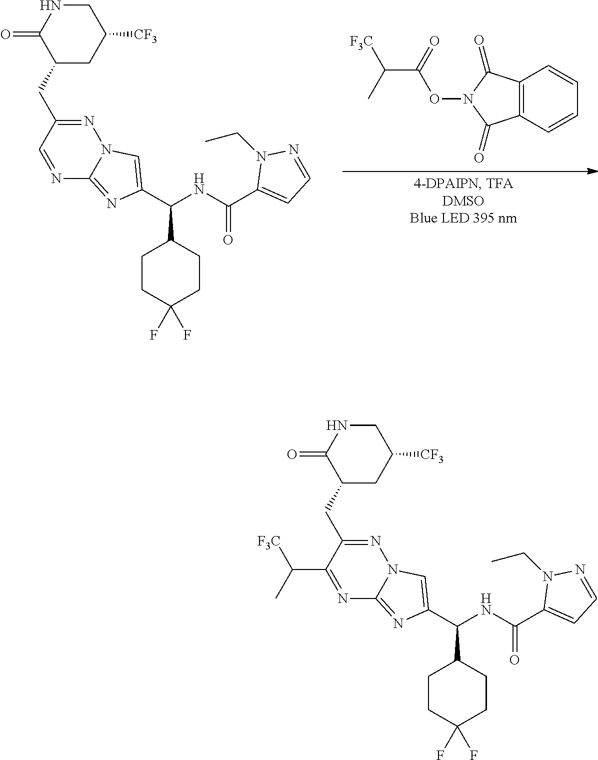

N-((1S)-(4,4-difluorocyclohexyl)(2-(((3R,5R)-2-oxo-5-(trifluoromethyl)piperidin-3-yl)methyl)-3-(1,1,1-trifluoropropan-2-yl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1,3-dioxoisoindolin-2-yl 3,3,3-trifluoro-2-methylpropanoate (121 mg, 422 μmol, 2.00 eq) and was purified by prep-HPLC (column: Phenomenex luna C18 150×25 mm, 10 μm; mobile phase: [water (FA)—acetonitrile]; gradient: 38%-68% B over 10 min) to afford N-((1S)-(4,4-difluorocyclohexyl)(2-(((3R,5R)-2-oxo-5-(trifluoromethyl)piperidin-3-yl)methyl)-3-(1,1,1-trifluoropropan-2-yl)imidazo[1,2-b][1,2,4]triazin-6-yl)methyl)-1-ethyl-1H-pyrazole-5-carboxamide, Compound 214 (40.0 mg, 60.2 μmol, 28.5% yield) as a yellow solid. LCMS [M+H]⁺=665.4 m/z.

Compound 174 was isolated as the first eluting, single stereoisomer by chiral SFC purification (column: Daicel Chiralcel OD-H (250 mm×30 mm, 5 μm); mobile phase: [CO₂-i-PrOH]; B %: 25%, isocratic elution mode); (8.89 mg, 10.09 μmol, 26.81% yield) and was obtained as a yellow solid. LCMS [M+H]⁺=665.5 m/z.

Compound 175 was isolated as the second eluting, single stereoisomer by chiral SFC purification (column: Daicel Chiralcel OD-H (250 mm×30 mm, 5 μm); mobile phase: [CO₂-i-PrOH]; B %: 25%, isocratic elution mode); (11.79 mg, 14.2 μmol, 37.6% yield) and was obtained as a yellow solid. LCMS [M+H]⁺=665.4 m/z.

### Example 134: Preparation of 1-ethyl-N-((1S)-((1R,4S)-4-methylcyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1H-pyrazole-5-carboxamide (Compound 179)

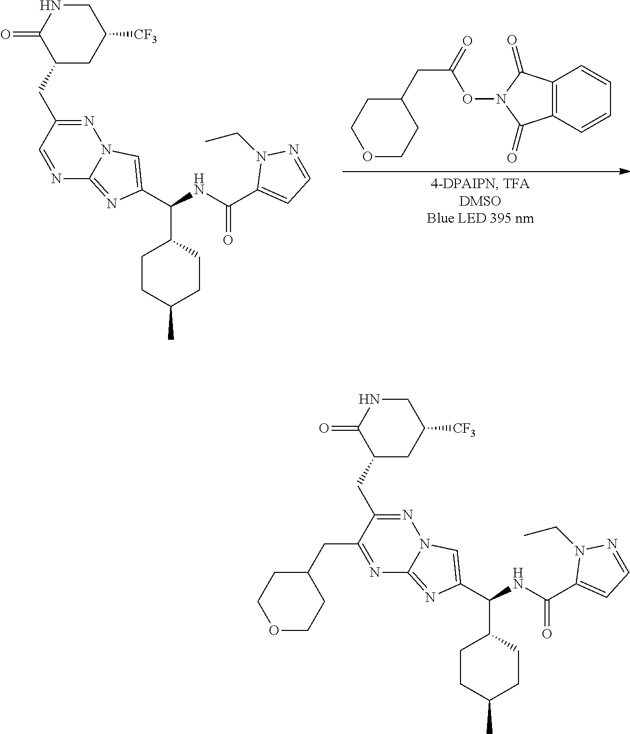 [SMILES: CCn1nccc1C(=O)N[C@H](c1cn2nc(C[C@H]3C[C@@H](C(F)(F)F)CNC3=O)cnc2n1)[C@H]1CC[C@H](C)CC1 ; O=C(CC1CCOCC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCOCC3)nc2n1)[C@H]1CC[C@H](C)CC1]

1-Ethyl-N-((1S)-((1R,4S)-4-methylcyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1H-pyrazole-5-carboxamide was synthesized following the General Procedure 7 with 1-ethyl-N-((1S)-((1R,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (80.0 mg, 146 μmol, 1.00 eq), 1,3-dioxoisoindolin-2-yl 2-(tetrahydro-2H-pyran-4-yl)acetate (84.7 mg, 293 μmol, 2.00 eq) and was purified by prep-HPLC (FA condition; column: Phenomenex luna C18 150×25 mm, 10 μm; mobile phase: [water (FA)—acetonitrile], gradient: 55%-85% B over 10 min) to afford 1-ethyl-N-((1S)-((1R,4S)-4-methylcyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1H-pyrazole-5-carboxamide, Compound 179 (47.77 mg, 70.91 μmol, 48.45% yield) as a light yellow solid. LCMS [M+H]⁺=645.3 m/z.

### Example 135: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(trifluoromethyl)-1H-pyrazole-5-carboxamide (Compound 184)

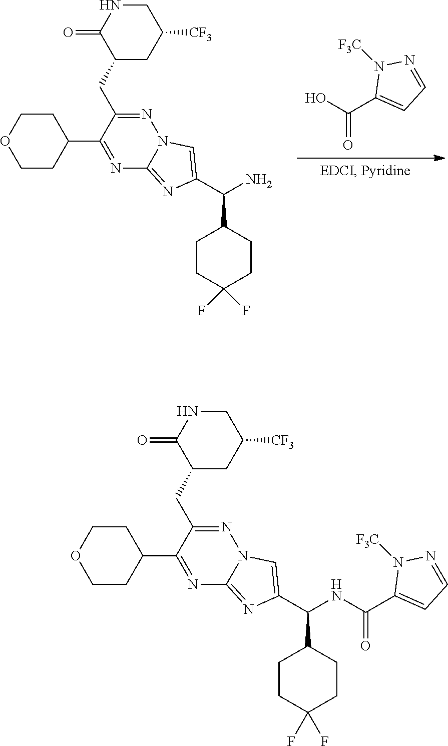

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(trifluoromethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 mol, 1.00 eq), 1-(trifluoromethyl)-1H-pyrazole-5-carboxylic acid (20.3 mg, 113 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(trifluoromethyl)-1H-pyrazole-5-carboxamide, Compound 184 (42.12 mg, 60.21 μmol, 63.8% yield) as a white solid. LCMS [M+H]⁺=693.3 m/z.

### Example 136: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(difluoromethyl)-1H-pyrazole-5-carboxamide (Compound 185)

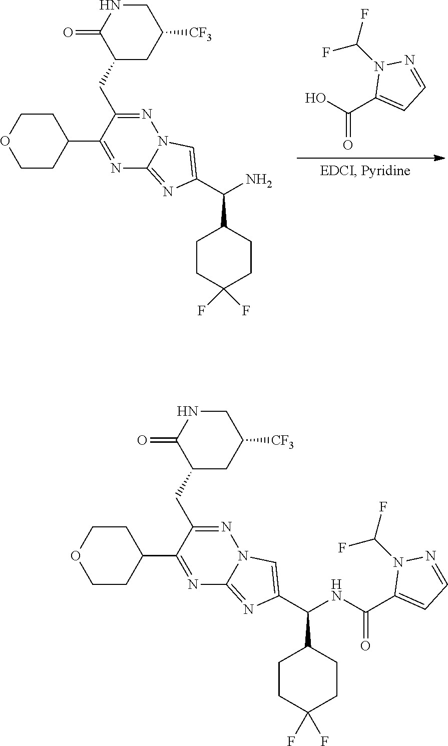

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(difluoromethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 mol, 1.00 eq), 1-(difluoromethyl)-1H-pyrazole-5-carboxylic acid (22.9 mg, 141 μmol, 1.50 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(difluoromethyl)-1H-pyrazole-5-carboxamide, Compound 185 (41.88 mg, 61.0 μmol, 64.7% yield) as a yellow solid. LCMS [M+H]⁺=675.3 m/z.

### Example 137: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2,2-trifluoroethyl)-1H-pyrazole-5-carboxamide (Compound 186)

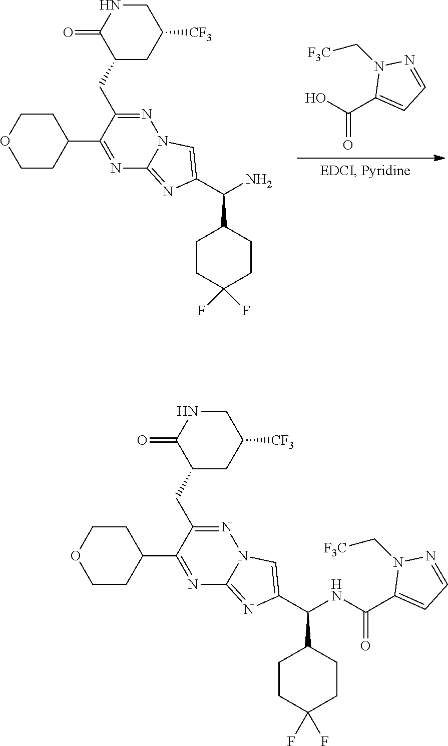

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2,2-trifluoroethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (45.0 mg, 84.8 μmol, 1.00 eq), 1-(2,2,2-trifluoroethyl)-1H-pyrazole-5-carboxylic acid (19.7 mg, 101 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2,2-trifluoroethyl)-1H-pyrazole-5-carboxamide, Compound 186 (44.39 mg, 62.1 μmol, 73.3% yield) as a yellow solid. LCMS [M+H]⁺=707.3 m/z.

### Example 138: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2-difluoroethyl)-1H-pyrazole-5-carboxamide (Compound 187)

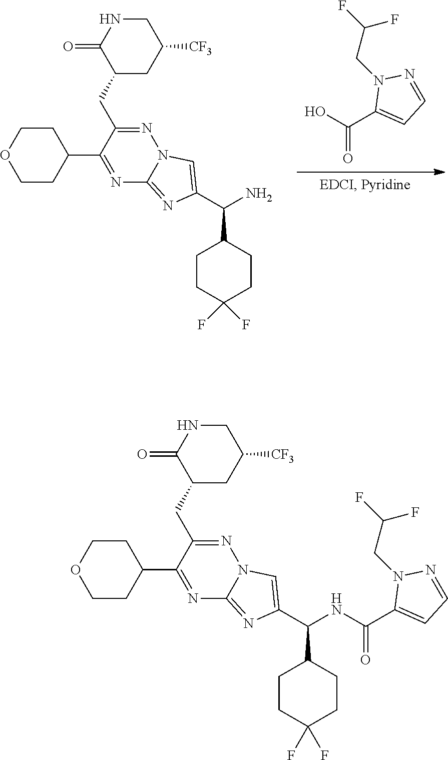

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2-difluoroethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (45.0 mg, 84.8 μmol, 1.00 eq), 1-(2,2-difluoroethyl)-1H-pyrazole-5-carboxylic acid (17.9 mg, 101 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2,2-difluoroethyl)-1H-pyrazole-5-carboxamide, Compound 187 (47.59 ng, 68.6 μmol. 80.9% yield) as a white solid. LCMS [M+H]⁺=689.3 m/z.

### Example 139: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-fluoroethyl)-1H-pyrazole-5-carboxamide (Compound 188)

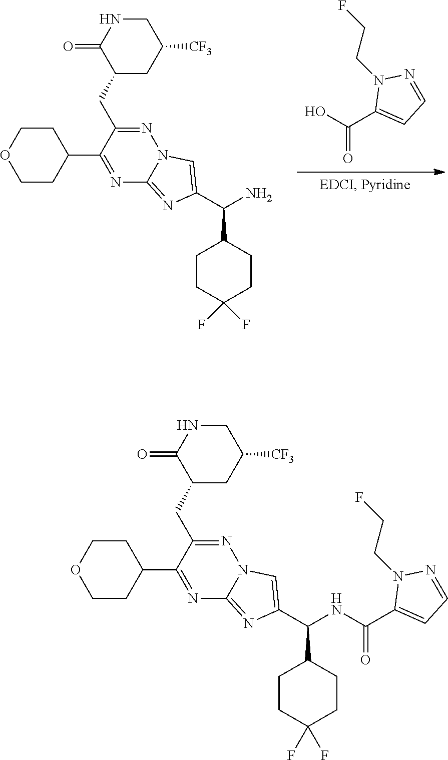

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-fluoroethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (45.0 mg, 84.8 μmol, 1.00 eq), 1-(2-fluoroethyl)-1H-pyrazole-5-carboxylic acid (16.1 mg, 101 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-fluoroethyl)-1H-pyrazole-5-carboxamide, Compound 188 (41.26 mg, 61.0 μmol, 72.0% yield) as a yellow solid. LCMS [M+H]⁺=671.2 m/z.

### Example 140: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylisoxazole-4-carboxamide (Compound 189)

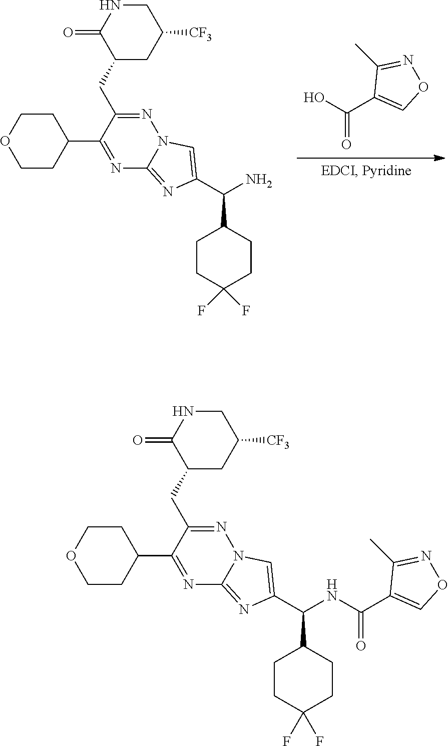

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylisoxazole-4-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (45.0 mg, 84.8 μmol, 1.00 eq), 3-methylisoxazole-4-carboxylic acid (11.8 mg, 93.3 μmol, 1.10 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylisoxazole-4-carboxamide, Compound 189 (49.78 mg, 77.5 μmol, 91.4% yield) as a yellow solid. LCMS [M+H]⁺=640.2 m/z.

### Example 141: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(difluoromethyl)-1,2,5-oxadiazole-3-carboxamide (Compound 190)

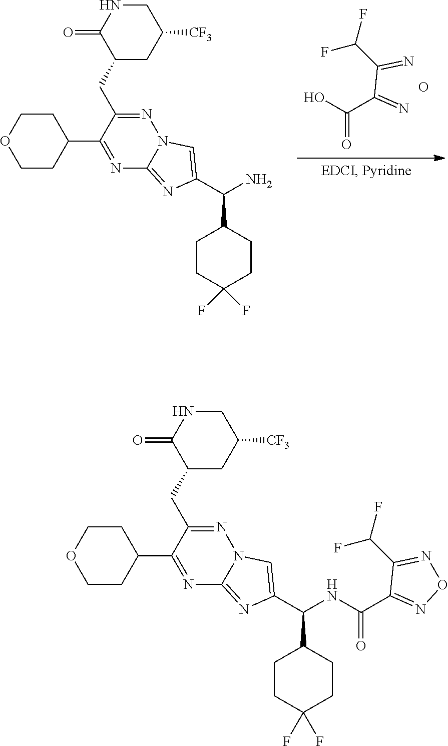

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(difluoromethyl)-1,2,5-oxadiazole-3-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 4-(difluoromethyl)-1,2,5-oxadiazole-3-carboxylic acid (14.8 mg, 90.4 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(difluoromethyl)-1,2,5-oxadiazole-3-carboxamide, Compound 190 (28.09 mg, 40.4 μmol, 53.5% yield) as a white solid. LCMS [M+H]⁺=677.4 m/z.

### Example 142: Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(trifluoromethyl)isoxazole-4-carboxamide (Compound 191)

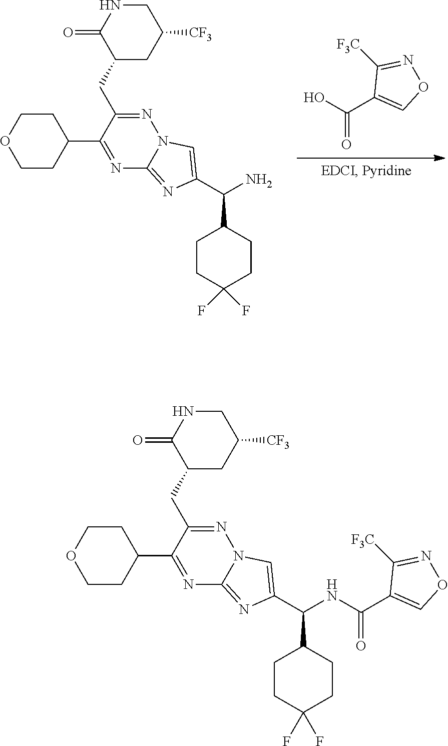

N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(trifluoromethyl)isoxazole-4-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 3-(trifluoromethyl)isoxazole-4-carboxylic acid (20.4 mg, 113 μmol, 1.20 eq) and was purified by prep-TLC (SiO₂, dichloromethane:methanol) to afford N-((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(trifluoromethyl)isoxazole-4-carboxamide, Compound 191 (50.0 mg, 71.3 μmol, 75.7% yield) as a white solid. LCMS [M+H]⁺=694.3 m/z.

### Example 143: Preparation of N-((1S)-(3-(but-3-en-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 215)

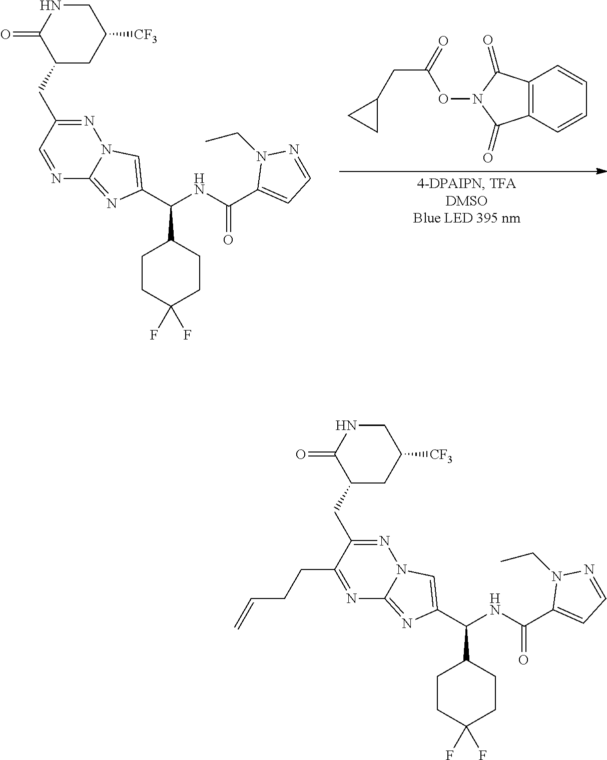 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CC1)ON1C(=O)c2ccccc2C1=O ; "4-DPAIPN, TFADMSOBlue LED 395 nm" ; C=CCCc1nc2nc([C@@H](NC(=O)c3ccnn3CC)C3CCC(F)(F)CC3)cn2nc1C[C@H]1C[C@@H](C(F)(F)F)CNC1=O]

N-((1S)-(3-(but-3-en-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 2-cyclopropylacetate (51.8 mg, 211 μmol, 2.00 eq) and was purified by prep-HPLC (column: Phenomenex luna C18 150×25 mm, 10 μm; mobile phase: [water (FA)—acetonitrile]; gradient: 43%-63% B over 10 min) to afford N-((1S)-(3-(but-3-en-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide, Compound 215 (33.8 mg, 54.3 μmol, 51.7% yield) as a white solid. LCMS [M+H]⁺=623.5 nm/z.

### Example 144: Preparation of N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide (Compounds 61 and 62)

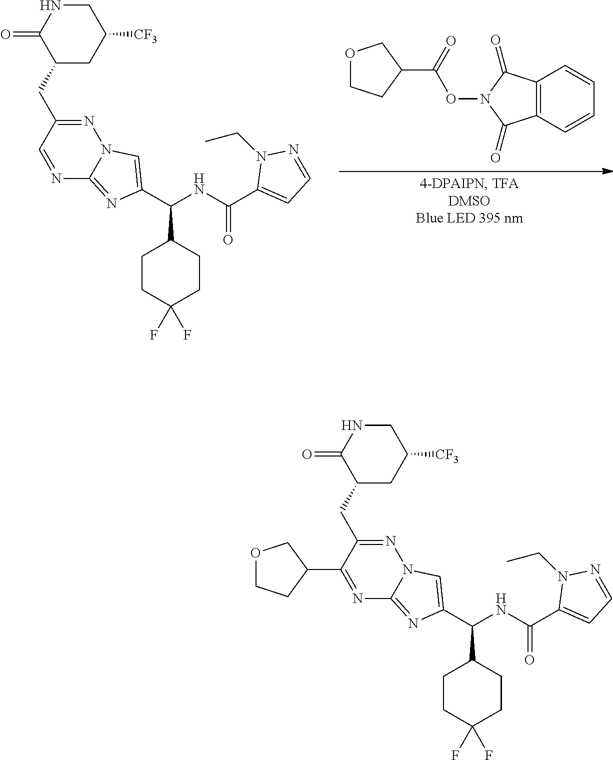 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-isopropyl-1H-pyrazole-5-carboxamide (1.00 eq), 1,3-dioxoisoindolin-2-yl tetrahydrofuran-3-carboxylate (2.00 eq) and was purified by prep-HPLC (column: Phenomenex luna C18 150×25 mm, 10 μm; mobile phase: [water (FA)—acetonitrile]) to afford N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-1-ethyl-1H-pyrazole-5-carboxamide as a white solid. LCMS [M+H]⁺=639.4 m/z.

Compound 61 was isolated as the first eluting, single stereoisomer by chiral SFC purification (column: Daicel Chiralcel OD-H (250 mm×30 mm, 5 μm); mobile phase: [CO₂-i-PrOH (0.1% NH₃H₂O)]; B %: 35%, isocratic elution mode); (28.8 mg, 39.1 μmol, 31.2% yield) and was obtained as a white solid. LCMS [M+H]⁺=639.4 m/z.

Compound 62 was isolated as the second eluting, single stereoisomer by chiral SFC purification (column: Daicel Chiralcel OD-H (250 mm×30 mm, 5 μm); mobile phase: [CO₂-i-PrOH (0.1% NH₃H₂O)]; B %: 35%, isocratic elution mode); (29.6 mg, 39.1 μmol, 31.2% yield) and was obtained as a white solid. LCMS [M+H]⁺=639.4 m/z.

### Example 145: Preparation of N-((1S)—((R)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 206)

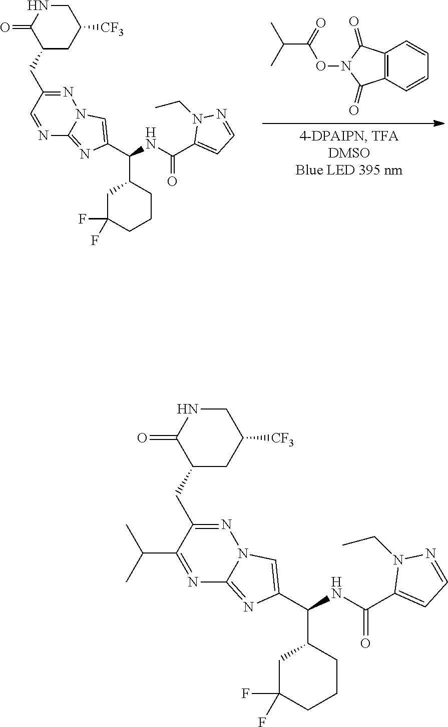 [SMILES: CCn1nccc1C(=O)N[C@H](c1cn2nc(C[C@H]3C[C@@H](C(F)(F)F)CNC3=O)cnc2n1)[C@@H]1CCCC(F)(F)C1 ; CC(C)C(=O)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)C)nc2n1)[C@@H]1CCCC(F)(F)C1]

N-((1S)—((R)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N-((1S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (80.0 mg, 140 μmol, 1.00 eq), 1,3-dioxoisoindolin-2-yl isobutyrate (65.6 mg, 281 μmol, 2.00 eq) and was purified by prep-HPLC (FA condition; column: Phenomenex luna C18 150×25 mm, 10 um; mobile phase: [water (FA)—acetonitrile]; gradient: 42%-72% B over 10 min) to afford N-((1S)—((R)-3,3-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 206 (46.85 mg, 73.1 μmol, 51.9% yield) as a white solid. LCMS [M+H]⁺=611.4 m/z.

### Example 146: Preparation of N—((S)-(4,4-difluorocyclohexyl)(3-(morpholinomethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 142)

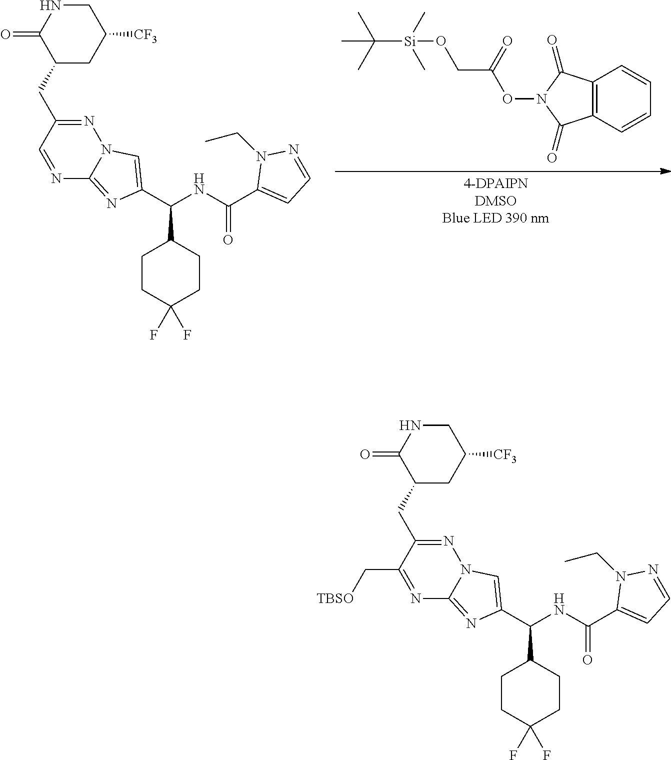 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)[Si](C)(C)OCC(=O)ON1C(=O)c2ccccc2C1=O ; "4-DPAIPNDMSOBlue LED 390 nm" ; CCn1nccc1C(=O)N[C@H](c1cn2nc(C[C@H]3C[C@@H](C(F)(F)F)CNC3=O)c(CO[Si](C)(C)C(C)(C)C)nc2n1)C1CCC(F)(F)CC1]

N-((1S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N-((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq), 1,3-dioxoisoindolin-2-yl 2-((tert-butyldimethylsilyl)oxy)acetate (118 mg, 351 μmol, 2.00 eq) and was purified by prep-HPLC (neutral condition; column: Waters Xbridge 150×25 mm, 10 μm; mobile phase: [water (NH₄HCO₃)—acetonitrile]; gradient: 55%-85% B over 10 min) to afford N-((1S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (30.0 mg, 42.0 μmol, 23.9% yield) as a yellow solid. LCMS [M+H]⁺=713.3 m/z.

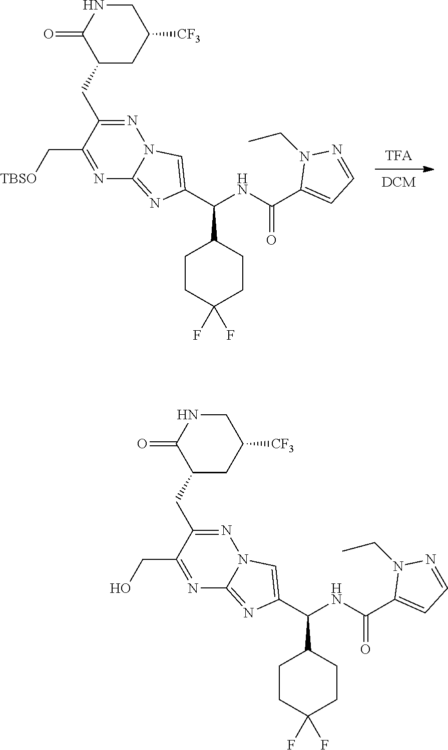

To a solution of N-((1S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (80.0 mg, 112 μmol, 1.00 eq) in dichloromethane (1.00 mL) was added TFA (767 mg, 6.73 mmol, 0.500 mL, 59.9 eq) at 0° C. The reaction mixture was stirred at 20° C. for 4 h. The pH of the reaction was adjusted to 8 with NaHCO₃. The reaction mixture was partitioned between ethyl acetate and water. The organic phase was separated, dried over anhydrous sodium hydrogen carbonate, filtered, and concentrated under reduced pressure to give a residue, which was purified by prep-TLC (SiO₂, dichloromethane:methanol) to afford N—((S)-(4,4-difluorocyclohexyl)(3-(hydroxymethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 83.5 μmol, 74.4% yield) as a yellow solid. LCMS [M+H]⁺=599.3 m/z.

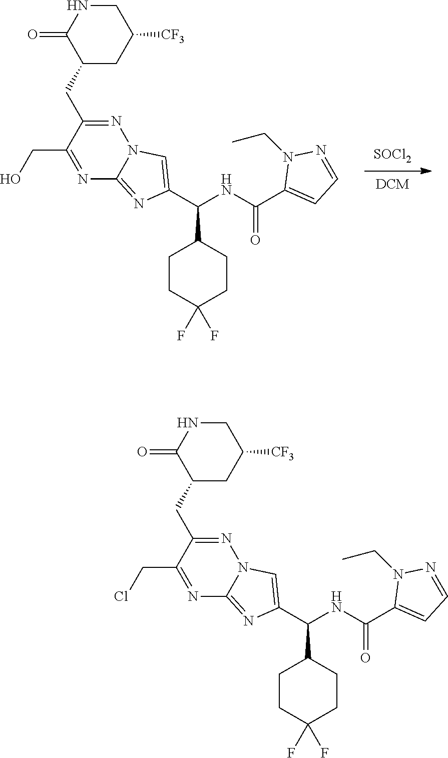

To a solution of N—((S)-(4,4-difluorocyclohexyl)(3-(hydroxymethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 83.5 μmol, 1.00 eq) in dichloromethane (0.50 mL) was added SOCl₂ (29.8 mg, 250 μmol, 18.2 μL, 3.00 eq) at 0° C. The reaction mixture was stirred at 20° C. for 0.5 h. The reaction was concentrated under reduced pressure to afford N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 77.6% yield) as a yellow solid. LCMS [M+H]⁺=617.3 m/z.

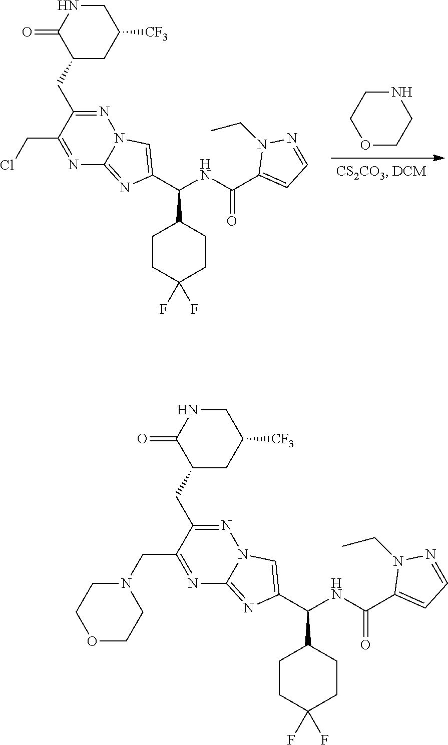

To a solution of N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) in dichloromethane (1.00 mL) was added morpholine (33.8 mg, 388 μmol, 34.2 μL, 6.00 eq). The reaction mixture was stirred at 20° C. for 1 h. The reaction mixture was concentrated under reduced pressure to give a residue, which was purified by prep-TLC (SiO₂, dichloromethane:methanol) to afford N—((S)-(4,4-difluorocyclohexyl)(3-(morpholinomethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 142 (23.69 mg, 34.0 μmol, 52.4% yield) as a light yellow solid. LCMS [M+H]⁺=668.4 m/z.

### Example 147: Preparation of N-((1S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 216)

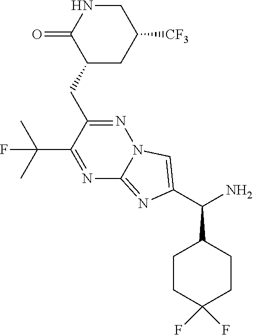 [SMILES: CC(C)(F)c1nc2nc([C@@H](N)C3CCC(F)(F)CC3)cn2nc1C[C@H]1C[C@@H](C(F)(F)F)CNC1=O]

(3R,5R)-3-((6-((S)-amino(4,4-difluorocyclohexyl)methyl)-3-(2-fluoropropan-2-yl)imidazo[1,2-b][1,2,4]triazin-2-yl)methyl)-5-(trifluoromethyl)piperidin-2-one was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate and 1,3-dioxoisoindolin-2-yl 2-fluoro-2-methylpropanoate, and General Procedure 5, employing benzyl ((1S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)carbamate.

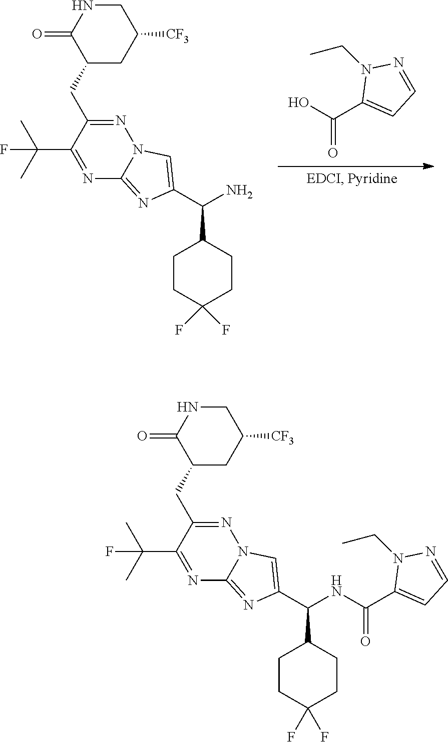

N-((1S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(2-fluoropropan-2-yl)imidazo[1,2-b][1,2,4]triazin-2-yl)methyl)-5-(trifluoromethyl)piperidin-2-one (34.0 mg, 67.1 μmol, 1.00 eq), 1-ethyl-1H-pyrazole-5-carboxylic acid (16.0 mg, 114.1 μmol, 1.70 eq) and was purified by prep-HPLC (column: Gemini C18 250×21.2 mm, 5 μm; mobile phase: [water (TFA)—acetonitrile]; gradient: 10%-60% B over 30 min) to afford N-((1S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 216 (9.45 mg, 14.3 μmol, 21.27% yield) as a white solid. LCMS [M+H]⁺=629.3 m/z.

### Example 148: Preparation of N-((1S)-(4,4-difluorocyclohexyl)(3-(2-fluoropropan-2-yl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 161)

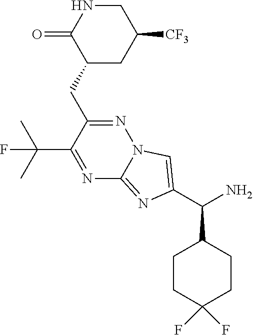 [SMILES: CC(C)(F)c1nc2nc([C@@H](N)C3CCC(F)(F)CC3)cn2nc1C[C@H]1C[C@H](C(F)(F)F)CNC1=O]

(3R,5S)-3-((6-((S)-amino(4,4-difluorocyclohexyl)methyl)-3-(2-fluoropropan-2-yl)imidazo[1,2-b][1,2,4]triazin-2-yl)methyl)-5-(trifluoromethyl)piperidin-2-one was synthesized according to General Procedure 3, employing (3R,5S)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate and 1,3-dioxoisoindolin-2-yl 2-fluoro-2-methylpropanoate, and General Procedure 5, employing benzyl ((1S)-(4,4-difluorocyclohexyl)(3-(2-fluoropropan-2-yl)-2-(((3R,5S)-2-oxo-5-(trifluoromethyl)piperidin-3-yl)methyl)imidazo[1,2-b][1,2,4]triazin-6-yl)methyl)carbamate.

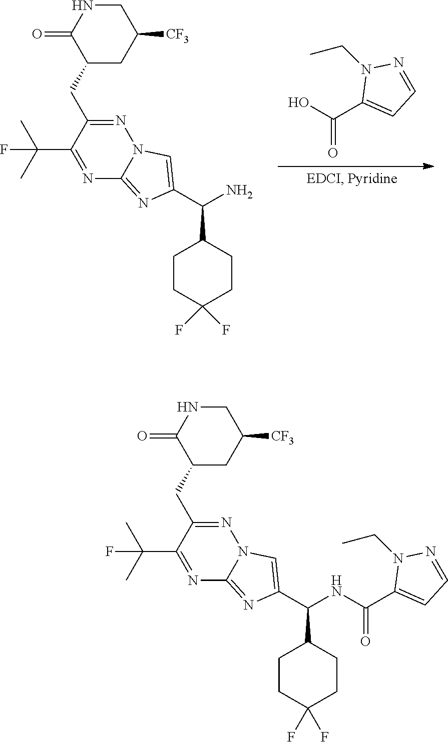

N-((1S)-(4,4-difluorocyclohexyl)(3-(2-fluoropropan-2-yl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with (3R,5S)-3-((6-((S)-amino(4,4-difluorocyclohexyl)methyl)-3-(2-fluoropropan-2-yl)imidazo[1,2-b][1,2,4]triazin-2-yl)methyl)-5-(trifluoromethyl)piperidin-2-one (25.0 mg, 49.4 μmol, 1.00 eq), 1-ethyl-1H-pyrazole-5-carboxylic acid (11.8 mg, 83.9 μmol, 1.70 eq) and was purified by prep-HPLC (column: Gemini C18 250×21.2 mm, 5 μm; mobile phase: [water (TFA)—acetonitrile]; gradient: 10%-60% B over 30 min) to afford N-((1S)-(4,4-difluorocyclohexyl)(3-(2-fluoropropan-2-yl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 161 (3.8 mg, 5.4 μmol, 11.02% yield) as a white solid. LCMS [M+H]⁺=629.3 m/z.

### Example 149: Preparation of N-((1S)-(4,4-difluorocyclohexyl)(3-(tert-butyl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 156)

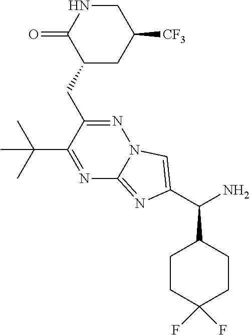 [SMILES: CC(C)(C)c1nc2nc([C@@H](N)C3CCC(F)(F)CC3)cn2nc1C[C@H]1C[C@H](C(F)(F)F)CNC1=O]

(3R,5S)-3-((6-((S)-amino(4,4-difluorocyclohexyl)methyl)-3-(tert-butyl)imidazo[1,2-b][1,2,4]triazin-2-yl)methyl)-5-(trifluoromethyl)piperidin-2-one was synthesized according to General Procedure 3, employing (3R,5S)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate and 1,3-dioxoisoindolin-2-yl pivalate, and General Procedure 5, employing benzyl ((1S)-(4,4-difluorocyclohexyl)(3-(tert-butyl)-2-(((3R,5S)-2-oxo-5-(trifluoromethyl)piperidin-3-yl)methyl)imidazo[1,2-b][1,2,4]triazin-6-yl)methyl)carbamate.

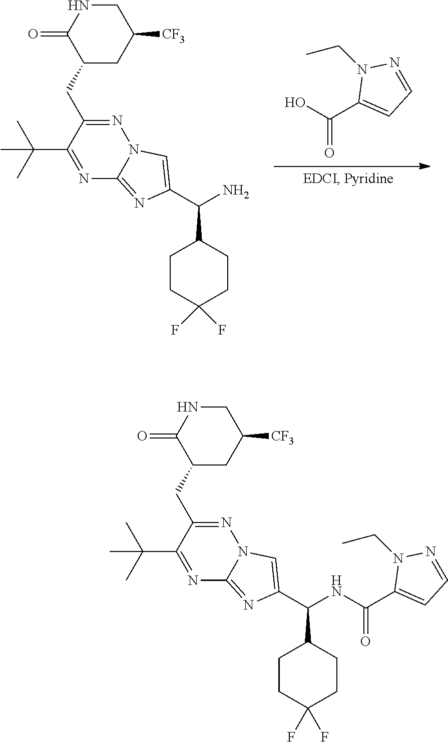

N-((1S)-(4,4-difluorocyclohexyl)(3-(tert-butyl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with (3R,5S)-3-((6-((S)-amino(4,4-difluorocyclohexyl)methyl)-3-(tert-butyl)imidazo[1,2-b][1,2,4]triazin-2-yl)methyl)-5-(trifluoromethyl)piperidin-2-one (29.0 mg, 57.7 μmol, 1.00 eq), 1-ethyl-1H-pyrazole-5-carboxylic acid (13.75 mg, 98.1 μmol, 1.70 eq) and was purified by prep-HPLC (column: Gemini C18 250×21.2 mm, 5 μm; mobile phase: [water (TFA)—acetonitrile]; gradient: 10%-60% B over 30 min) to afford N-((1S)-(4,4-difluorocyclohexyl)(3-(tert-butyl)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 156 (7.2 mg, 10.6 μmol, 18.38% yield) as a white solid. LCMS [M+H]⁺=625.3 m/z.

### Example 150: Preparation of N-((1S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 182)

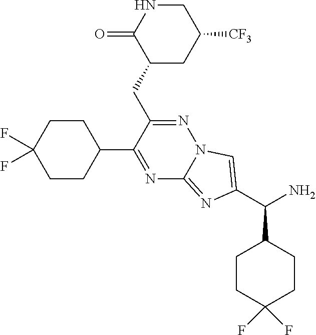 [SMILES: N[C@H](c1cn2nc(C[C@H]3C[C@@H](C(F)(F)F)CNC3=O)c(C3CCC(F)(F)CC3)nc2n1)C1CCC(F)(F)CC1]

(3R,5R)-3-((6-((S)-amino(4,4-difluorocyclohexyl)methyl)-3-(4,4-difluorocyclohexyl)imidazo[1,2-b][1,2,4]triazin-2-yl)methyl)-5-(trifluoromethyl)piperidin-2-one was synthesized according to General Procedure 3, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate and 1,3-dioxoisoindolin-2-yl 4,4-difluorocyclohexane-1-carboxylate, and General Procedure 5, employing benzyl ((1S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)carbamate.

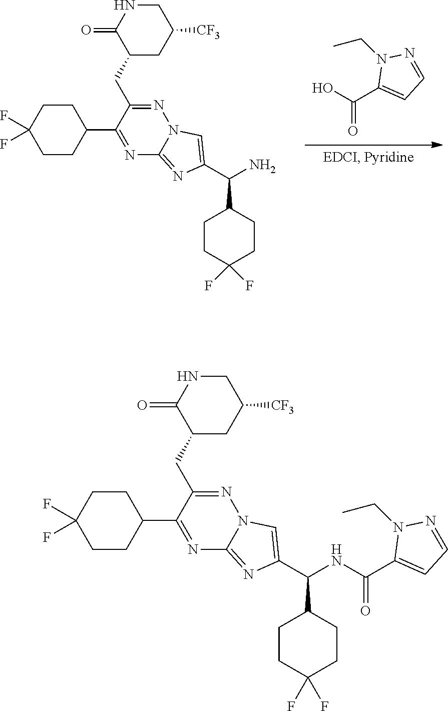

N-((1S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with ((3R,5R)-3-((6-((S)-amino(4,4-difluorocyclohexyl)methyl)-3-(4,4-difluorocyclohexyl)imidazo[1,2-b][1,2,4]triazin-2-yl)methyl)-5-(trifluoromethyl)piperidin-2-one (75.0 mg, 132.9 μmol, 1.00 eq), 1-ethyl-1H-pyrazole-5-carboxylic acid (31.65 mg, 225.8 μmol, 1.70 eq) and was purified by prep-HPLC (column: Gemini C18 250×21.2 mm, 5 μm; mobile phase: [water (TFA)—acetonitrile]; gradient: 10%-60% B over 30 min) to afford N-((1 S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide, Compound 182 (26.27 mg, 36.0 μmol, 27.07% yield) as a white solid. LCMS [M+H]⁺=687.3 m/z.

### Example 151: Preparation of (5-(trifluoromethyl)isoxazol-3-yl)methyl ((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate (Compound 183)

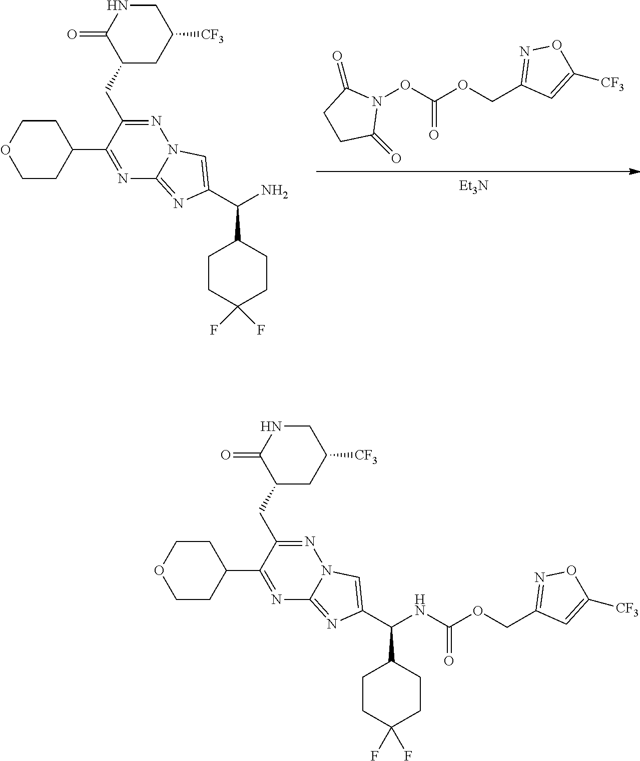 [SMILES: "Et3N" ; N[C@H](c1cn2nc(C[C@H]3C[C@@H](C(F)(F)F)CNC3=O)c(C3CCOCC3)nc2n1)C1CCC(F)(F)CC1 ; O=C(OCc1cc(C(F)(F)F)on1)ON1C(=O)CCC1=O ; O=C(N[C@H](c1cn2nc(C[C@H]3C[C@@H](C(F)(F)F)CNC3=O)c(C3CCOCC3)nc2n1)C1CCC(F)(F)CC1)OCc1cc(C(F)(F)F)on1]

(5-(trifluoromethyl)isoxazol-3-yl)methyl ((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 75.4 μmol, 1.00 eq), 2,5-dioxopyrrolidin-1-yl ((5-(trifluoromethyl)isoxazol-3-yl)methyl) carbonate (34.85 mg, 113.1 μmol, 1.50 eq) and was purified by prep-HPLC (column: Gemini C18 250×21.2 mm, 5 μm; mobile phase: [water (TFA)—acetonitrile]; gradient: 10%-60% B over 30 min) to afford (5-(trifluoromethyl)isoxazol-3-yl)methyl ((1S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate, Compound 183 (23.18 mg, 28.8 μmol, 38.24% yield) as a yellow solid. LCMS [M+H]⁺=724.2 m/z.

### Example 152: Preparation of N-((1S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 196)

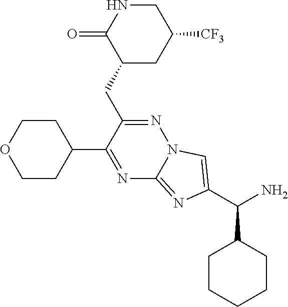 [SMILES: N[C@H](c1cn2nc(C[C@H]3C[C@@H](C(F)(F)F)CNC3=O)c(C3CCOCC3)nc2n1)C1CCCCC1]

(3R,5R)-3-((6-((S)-amino(cyclohexyl)methyl)-3-(tetrahydro-2H-pyran-4-yl)imidazo[1,2-b][1,2,4]triazin-2-yl)methyl)-5-(trifluoromethyl)piperidin-2-one was synthesized according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one and benzyl (S)-(3-bromo-1-cyclohexyl-2-oxopropyl)carbamate, and General Procedure 4, employing benzyl ((1S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate.

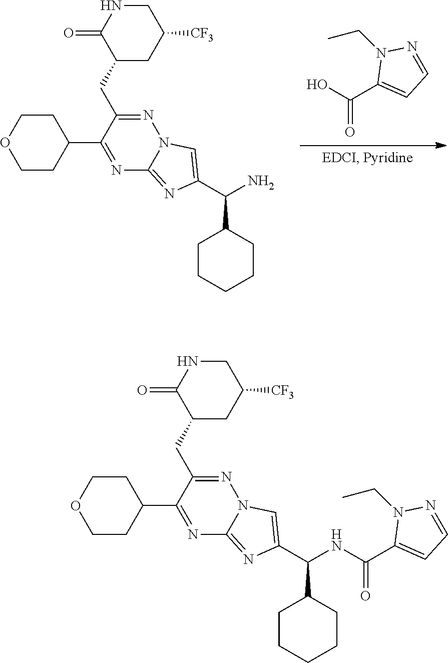

N-((1S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (30.0 mg, 60.6 μmol, 1.00 eq), 1-ethyl-1H-pyrazole-5-carboxylic acid (10.2 mg, 72.7 μmol, 1.20 eq) and was purified by prep-TLC (SiO₂, dichloromethane:methanol) to afford N-((1S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide, Compound 196 (26.06 mg, 40.7 μmol, 67.0% yield) as a white solid. LCMS [M+H]⁺=617.3 m/z.

### Example 153: Preparation of N—((S)-(4,4-difluorocyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 151)

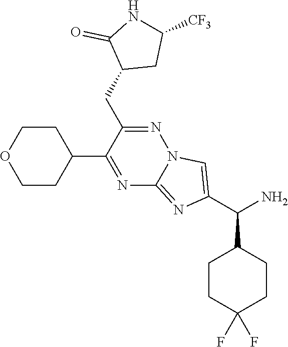 [SMILES: N[C@H](c1cn2nc(C[C@H]3C[C@@H](C(F)(F)F)NC3=O)c(C3CCOCC3)nc2n1)C1CCC(F)(F)CC1]

(3R,5S)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)pyrrolidin-2-one was synthesized according to General Procedure 3, employing (3R,5S)-3-((3-amino-5-(tetrahydro-2H-pyran-4-yl)-1,2,4-triazin-6-yl)methyl)-5-(trifluoromethyl)pyrrolidin-2-one and benzyl (S)-(3-bromo-1-(4,4-difluorocyclohexyl)-2-oxopropyl)carbamate, and General Procedure 4, employing benzyl ((S)-(4,4-difluorocyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)carbamate.

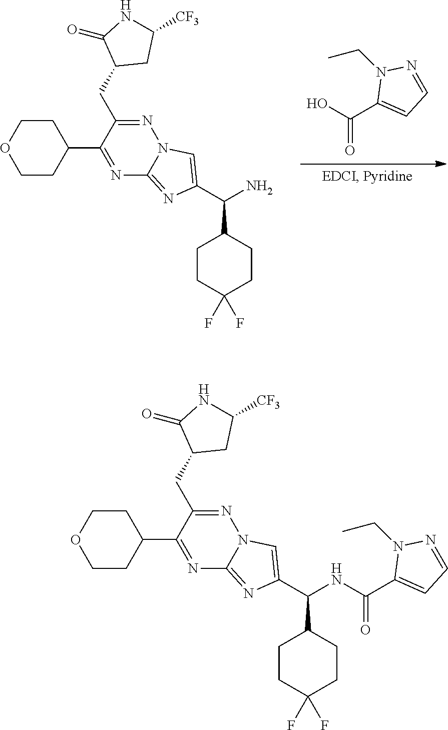

N—((S)-(4,4-difluorocyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (70.0 mg, 135 μmol, 1.00 eq), 1-ethyl-1H-pyrazole-5-carboxylic acid (22.7 mg, 162 μmol, 1.20 eq) and was purified by prep-HPLC (neutral condition; column: Waters Xbridge 150×25 mm, 5 μm; mobile phase: [water (NH₄HCO₃)—acetonitrile]; gradient: 27%-57% B over 10 min) to afford N—((S)-(4,4-difluorocyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1-ethyl-1H-pyrazole-5-carboxamide, Compound 151 (21.93 mg, 33.45 μmol, 24.68% yield) as a yellow solid. LCMS [M+H]⁺=639.4 m/z.

### Example 154: Preparation of 1-ethyl-N—((S)-((1R,4S)-4-methylcyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1H-pyrazole-5-carboxamide (Compound 152)

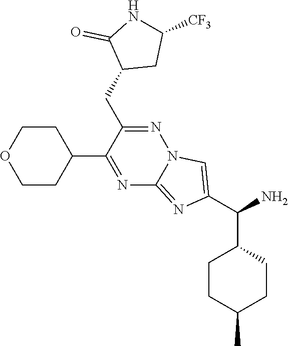 [SMILES: C[C@H]1CC[C@H]([C@H](N)c2cn3nc(C[C@H]4C[C@@H](C(F)(F)F)NC4=O)c(C4CCOCC4)nc3n2)CC1]

(3R,5S)-3-((6-((S)-amino((1R,4S)-4-methylcyclohexyl)methyl)-3-(tetrahydro-2H-pyran-4-yl)imidazo[1,2-b][1,2,4]triazin-2-yl)methyl)-5-(trifluoromethyl)pyrrolidin-2-one was synthesized according to General Procedure 3, employing (3R,5S)-3-((3-amino-5-(tetrahydro-2H-pyran-4-yl)-1,2,4-triazin-6-yl)methyl)-5-(trifluoromethyl)pyrrolidin-2-one and benzyl ((S)-3-bromo-1-((1R,4S)-4-methylcyclohexyl)-2-oxopropyl)carbamate, and General Procedure 4, employing benzyl ((S)-((1r,4S)-4-methylcyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)carbamate.

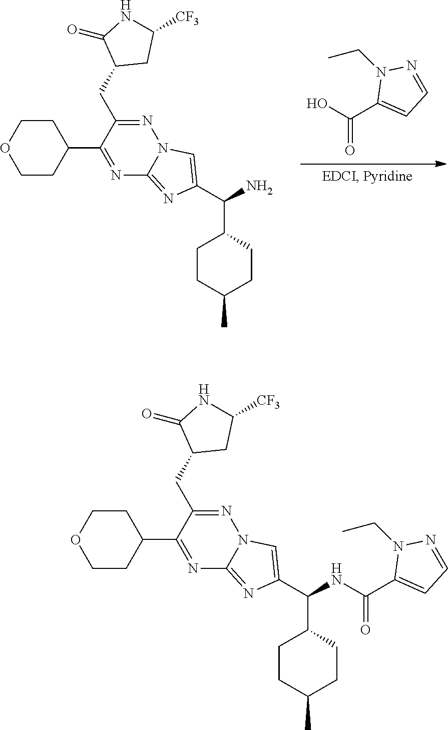

1-Ethyl-N—((S)-((1r,4S)-4-methylcyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1H-pyrazole-5-carboxamide was synthesized following the General Procedure 6 with (3R,5S)-3-((6-((S)-amino((1r,4S)-4-methylcyclohexyl)methyl)-3-(tetrahydro-2H-pyran-4-yl)imidazo[1,2-b][1,2,4]triazin-2-yl)methyl)-5-(trifluoromethyl)pyrrolidin-2-one (60.0 mg, 121 mol, 1.00 eq), 1-ethyl-1H-pyrazole-5-carboxylic acid (51.0 mg, 363 μmol, 3.00 eq) and was purified by prep-HPLC (column: Phenomenex Luna C18 150×25 mm, 10 μm; mobile phase: [water (HCl)—acetonitrile]; gradient: 45%-75% B over 9 min) to afford 1-ethyl-N—((S)-((1r,4S)-4-methylcyclohexyl)(2-(((3R,5S)-2-oxo-5-(trifluoromethyl)pyrrolidin-3-yl)methyl)-3-(tetrahydro-2H-pyran-4-yl)imidazo[1,2-b][1,2,4]triazin-6-yl)methyl)-1H-pyrazole-5-carboxamide, Compound 152 (15.5 mg, 25.1 μmol, 20.7% yield) as a yellow solid. LCMS [M+H]⁺=617.4 m/z.

### Example 155: Preparation of 1-ethyl-N-(1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)-2-((1,1,1-trifluoro-2-methylpropan-2-yl)oxy)ethyl)-1H-pyrazole-5-carboxamide (Compound 159)

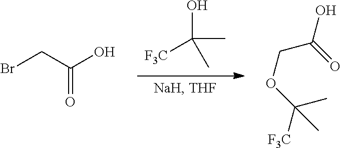

To a solution of 1,1,1-trifluoro-2-methylpropan-2-ol (38.4 g, 299 mmol, 32.8 mL, 1.00 eq) in THF (600 mL) was added NaH (23.9 g, 599 mmol, 60.0% purity, 2.00 eq) at 0° C. The reaction mixture was stirred at 0° C. for 1 h. A solution of 2-bromoacetic acid (50.0 g, 359 mmol, 25.8 mL, 1.20 eq) was added. The reaction mixture was stirred at 70° C. for 16 h. The reaction mixture was quenched by addition NH₄C₁ at 0° C., and then diluted with water and extracted with ethyl acetate. The combined organic layers were dried over by anhydrous sodium sulfate, filtered, and concentrated under reduced pressure to give a residue, which was purified by column chromatography (SiO₂, petroleum ether:ethyl acetate=100:1 to 1:1) to afford 2-((1,1,1-trifluoro-2-methylpropan-2-yl)oxy)acetic acid (34.0 g, 182 mmol, 60.9% yield) as a yellow oil. ¹H NMR (400 MHz, CDCl₃) δ 10.4 (s, 1H), 4.23 (s, 2H), 1.41 (s, 6H).

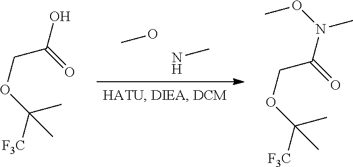

To a solution of 2-((1,1,1-trifluoro-2-methylpropan-2-yl)oxy)acetic acid (32.0 g, 171 mmol, 1.00 eq), HATU (130 g, 343 mmol, 2.00 eq) and DIEA (88.8 g, 687 mmol, 119 mL, 4.00 eq) in CH₂Cl₂ (600 mL) was added N,O-dimethylhydroxylamine (20.1 g, 206 mmol, 1.20 eq, HCl salt) at room temperature. The reaction mixture was stirred at room temperature for 2 h. The reaction mixture was diluted with water and extracted with ethyl acetate. The combined organic layers were dried over by anhydrous sodium sulfate, filtered, and concentrated under reduced pressure to give a residue, which was purified by column chromatography (SiO₂, Petroleum ether:Ethyl acetate=100:1 to 1:1) to afford N-methoxy-N-methyl-2-((1,1,1-trifluoro-2-methylpropan-2-yl)oxy)acetamide (27.0 g, 117 mmol, 68.5% yield) as a colorless oil.

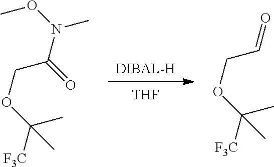

To a solution of N-methoxy-N-methyl-2-((1,1,1-trifluoro-2-methylpropan-2-yl)oxy)acetamide (27.0 g, 117 mmol, 1.00 eq) in THF (100 mL) was added DIBAL-H (1 M, 236 mL, 2.00 eq) at −75° C. The reaction mixture was stirred at −75° C. for 2 h. The reaction mixture was quenched by addition of water at 0° C., and then diluted with water and extracted with ethyl acetate.

The combined organic layers were dried over by anhydrous sodium sulfate, filtered, and concentrated under reduced pressure to afford 2-((1,1,1-trifluoro-2-methylpropan-2-yl)oxy)acetaldehyde (22.0 g, crude) as a yellow oil.

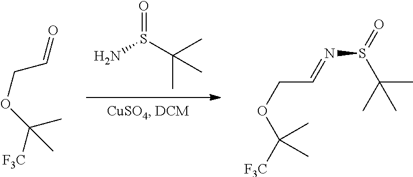

To a solution of 2-((1,1,1-trifluoro-2-methylpropan-2-yl)oxy)acetaldehyde (22.0 g, 129 mmol, 1.00 eq) and (S)-2-methylpropane-2-sulfinamide (18.8 g, 155 mmol, 1.20 eq) in dichloromethane (300 mL) was added CuSO₄ (41.2 g, 258 mmol, 39.6 mL, 2.00 eq) at room temperature. The reaction mixture was stirred at room temperature for 12 h. The reaction mixture was filtered and concentrated under reduced pressure to give a residue, which was purified by column chromatography (SiO₂, petroleum ether:ethyl acetate=100:1 to 1:1) to afford (S)-2-methyl-N-(2-((1,1,1-trifluoro-2-methylpropan-2-yl)oxy)ethylidene)propane-2-sulfinamide (6.00 g, 18.6 mmol, 14.4% yield) as a colorless oil. ¹H NMR (400 MHz, CDCl₃) δ 7.97 (s, 1H), 4.37 (s, 2H), 1.33 (s, 6H), 1.12 (s, 9H). LCMS [M+H]⁺=274.1 m/z.

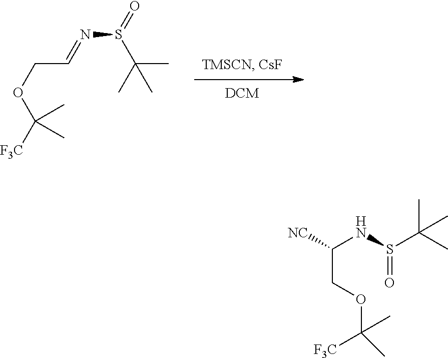

To a solution of (S)-2-methyl-N-(2-((1,1,1-trifluoro-2-methylpropan-2-yl)oxy)ethylidene)propane-2-sulfinamide (5.00 g, 18.2 mmol, 1.00 eq) in dichloromethane (50.0 mL) was added CsF (555 mg, 3.66 mmol, 135 μL, 0.200 eq), water (659 mg, 36.5 mmol, 659 μL, 2.00 eq) and TMSCN (3.63 g, 36.5 mmol, 4.58 mL, 2.00 eq). The reaction mixture was stirred at room temperature for 1 h. The reaction mixture was concentrated under reduced pressure and the residue was diluted with water and extracted with petroleum ether. The combined organic layers were dried over by anhydrous sodium sulfate, filtered, and concentrated under reduced pressure to give a residue, which was purified by column chromatography (SiO₂, petroleum ether:ethyl acetate=100:1 to 1:1) to afford (R)—N—((S)-1-cyano-2-((1,1,1-trifluoro-2-methylpropan-2-yl)oxy)ethyl)-2-methylpropane-2-sulfinamide (3.00 g, 9.77 mmol, 53.4% yield) as a yellow oil. ¹H NMR (400 MHz, CDCl₃) δ 5.27 (s, 1H), 4.43-4.31 (m, 2H), 3.84-3.73 (m, 2H), 1.38 (s, 6H), 1.22-1.20 (m, 9H). LCMS [M+H]⁺=301.1 m/z.

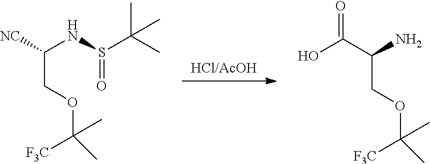 [SMILES: CC(C)(C)[S@@](=O)N[C@@H](C#N)COC(C)(C)C(F)(F)F ; "HCl/AcOH" ; CC(C)(OC[C@H](N)C(=O)O)C(F)(F)F]

To a solution of (R)—N—((S)-1-cyano-2-((1,1,1-trifluoro-2-methylpropan-2-yl)oxy)ethyl)-2-methylpropane-2-sulfinamide (3.00 g, 9.99 mmol, 1.00 eq) in acetic acid (15.7 g, 262 mmol, 15.0 mL, 26.2 eq) was added HCl (12 M, 15.0 mL, 18.0 eq). The reaction mixture was stirred at 110° C. for 12 h. The reaction mixture was concentrated under reduced pressure to afford O-(1,1,1-trifluoro-2-methylpropan-2-yl)-L-serine (2.50 g, 9.94 mmol, 99.4% yield, HCl salt) as a brown solid. LCMS [M+H]⁺=282.4 m/z.

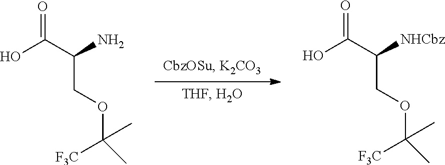

To a solution of O-(1,1,1-trifluoro-2-methylpropan-2-yl)-L-serine (2.50 g, 9.94 mmol, 1.00 eq, HCl salt), CbzOSu (2.48 g, 9.94 mmol, 1.00 eq) and potassium carbonate (4.12 g, 29.8 mmol, 3.00 eq) in THF (15.0 mL) was added water (15.0 mL). The reaction mixture was stirred at room temperature for 1 h. The reaction mixture was diluted with water and extracted with ethyl acetate. The combined organic layers were dried over by anhydrous sodium sulfate, filtered, and concentrated under reduced pressure to give a residue, which was purified by column chromatography (SiO₂, petroleum ether:ethyl acetate=100:1 to 1:1) to afford N-((benzyloxy)carbonyl)-O-(1,1,1-trifluoro-2-methylpropan-2-yl)-L-serine (2.50 g, 7.16 mmol, 72.0% yield) as a yellow oil. ¹H NMR (400 MHz, CDCl₃) δ 9.57 (s, 1H), 7.38-7.36 (m, 5H), 5.67-5.64 (m, 1H), 5.15-5.12 (m, 2H), 4.57-4.55 (m, 1H), 4.07-4.05 (m, 1H), 3.82-3.79 (m, 1H), 1.44-1.31 (m, 6H). LCMS [M+H]⁺=350.2 m/z.

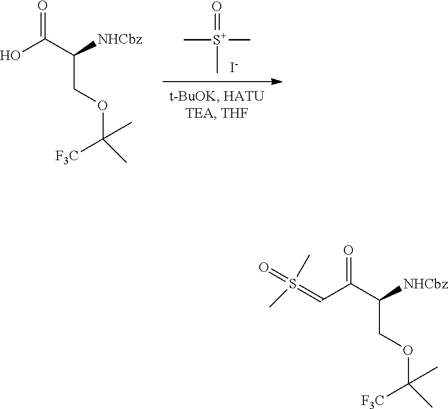

The preparation of mixture A: trimethylsulfoxonium iodide (4.44 g, 20.1 mmol, 3.00 eq) was added to a solution of t-BuOK (2.26 g, 20.1 mmol, 3.00 eq) in THF (30.0 mL) and the reaction mixture was stirred at 65° C. for 2 h under N₂. After that time, the solution was cooled to 0° C. to obtain the mixture A.

The preparation of mixture B: To a solution of N-((benzyloxy)carbonyl)-O-(1,1,1-trifluoro-2-methylpropan-2-yl)-L-serine (2.35 g, 6.73 mmol, 1.00 eq) in THF (30.0 mL) was added TEA (1.36 g, 13.4 mmol, 1.87 mL, 2.00 eq) and HATU (5.12 g, 13.4 mmol, 2.00 eq) at room temperature. The reaction mixture was stirred at room temperature for 2 h. The suspension was then filtered, and the filtrate was cooled to 0° C. to obtain the mixture B.

The mixture B was added to the mixture A at 0° C. The resulting mixture was stirred at room temperature for 1 h. The reaction mixture was quenched by the addition of water at room temperature, and extracted with ethyl acetate. The combined organic layers were dried over anhydrous sodium sulfate, filtered, and concentrated under reduced pressure to give a residue, which was purified by prep-TLC (SiO₂, dichloromethane:MeOH=10:1) to afford benzyl (S)-(4-(dimethyl(oxo)-λ6-sulfaneylidene)-3-oxo-1-((1,1,1-trifluoro-2-methylpropan-2-yl)oxy)butan-2-yl)carbamate (850 mg, 1.85 mmol, 27.4% yield) as a white solid. ¹H NMR (400 MHz, CDCl₃) δ 7.37-7.33 (m, 5H), 5.72-5.70 (m, 1H), 5.11-5.10 (m, 2H), 4.24-4.22 (m, 1H), 3.90-3.87 (m, 1H), 3.63-3.60 (m, 1H), 3.38-3.28 (m, 6H), 2.61 (s, 1H), 1.34-1.31 (m, 6H). LCMS [M+H]⁺=424.1 m/z.

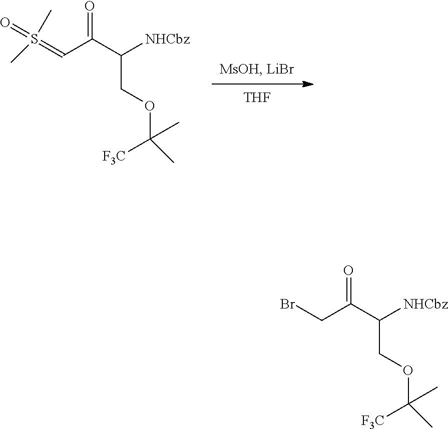

To a solution benzyl (4-(dimethyl(oxo)-λ6-sulfaneylidene)-3-oxo-1-((1,1,1-trifluoro-2-methylpropan-2-yl)oxy)butan-2-yl)carbamate (600 mg, 1.42 mmol, 1.00 eq) in THF (5.00 mL) was added LiBr (246 mg, 2.83 mmol, 71.1 μL, 2.00 eq) and MsOH (408 mg, 4.25 mmol, 303 μL, 3.00 eq) at 0° C. The reaction mixture was stirred at room temperature for 2 h. The reaction mixture was diluted with water and extracted with ethyl acetate. The combined organic layers were dried over by anhydrous sodium sulfate, filtered, and concentrated under reduced pressure to afford benzyl (4-bromo-3-oxo-1-((1,1,1-trifluoro-2-methylpropan-2-yl)oxy)butan-2-yl)carbamate (600 mg, crude) as a black oil.

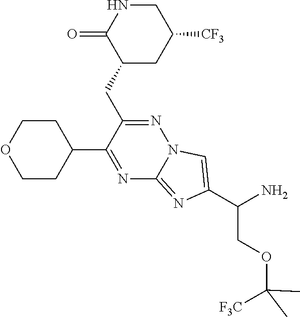 [SMILES: CC(C)(OCC(N)c1cn2nc(C[C@H]3C[C@@H](C(F)(F)F)CNC3=O)c(C3CCOCC3)nc2n1)C(F)(F)F]

(3R,5R)-3-((6-(1-amino-2-((1,1,1-trifluoro-2-methylpropan-2-yl)oxy)ethyl)-3-(tetrahydro-2H-pyran-4-yl)imidazo[1,2-b][1,2,4]triazin-2-yl)methyl)-5-(trifluoromethyl)piperidin-2-one was synthesized according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one and benzyl (4-bromo-3-oxo-1-((1,1,1-trifluoro-2-methylpropan-2-yl)oxy)butan-2-yl)carbamate, and General Procedure 4, employing benzyl (1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)-2-((1,1,1-trifluoro-2-methylpropan-2-yl)oxy)ethyl)carbamate.

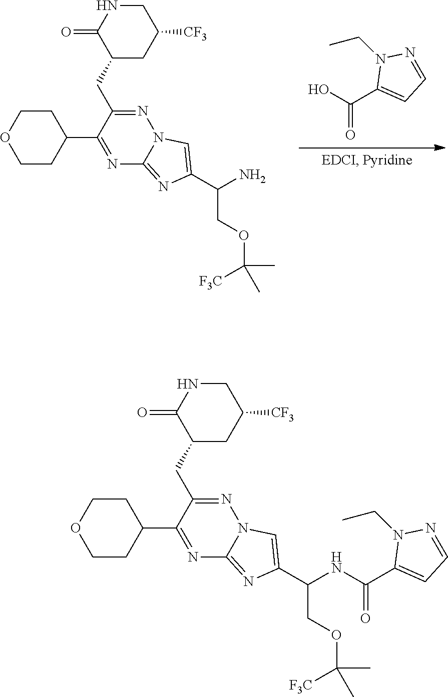

1-Ethyl-N-(1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)-2-((1,1,1-trifluoro-2-methylpropan-2-yl)oxy)ethyl)-1H-pyrazole-5-carboxamide was synthesized following the General Procedure 6 with (3R,5R)-3-((6-(1-amino-2-((1,1,1-trifluoro-2-methylpropan-2-yl)oxy)ethyl)-3-(tetrahydro-2H-pyran-4-yl)imidazo[1,2-b][1,2,4]triazin-2-yl)methyl)-5-(trifluoromethyl)piperidin-2-one (23.3 mg, 166 μmol, 2.00 eq), 1-ethyl-1H-pyrazole-5-carboxylic acid (27.9 mg, 199.2 μmol, 1.20 eq) and was purified by prep-TLC (SiO₂, dichloromethane:methanol) to afford 1-ethyl-N-(1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)-2-((1,1,1-trifluoro-2-methylpropan-2-yl)oxy)ethyl)-1H-pyrazole-5-carboxamide (30.0 mg, 44.4 μmol, 53.4% yield) as a yellow solid. LCMS [M+H]⁺=675.4 m/z. Compound 159 25201 was isolated as the first eluting, single stereoisomer by chiral SFC purification (column: Daicel Chiralpak IK (250 mm×30 mm, 10 μm); mobile phase: [CO₂—CAN/EtOH (0.1% NH₃H₂O)]; B %:50%, isocratic elution mode); (7.63 mg, 11.3 μmol, 25.4% yield) and was obtained as a white solid. LCMS [M+H]⁺=675.4 m/z.

### Example 156: Preparation of 1-ethyl-N-((1S)-5,5,5-trifluoro-4,4-dimethyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)pentyl)-1H-pyrazole-5-carboxamide (Compounds 160 and 217)

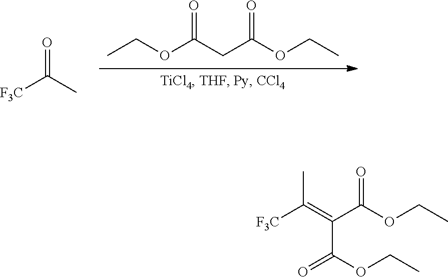

A mixture of THF (1080 mL) and CCl₄ (135 mL) was cooled to 0° C. and treated with TiCl₄ (101 g, 535 mmol, 2.00 eq), then was added 1,1,1-trifluoropropan-2-one (30.0 g, 268 mmol, 24.0 mL, 1.00 eq) and diethyl malonate (42.9 g, 268 mmol, 40.6 mL, 1.00 eq). The reaction mixture was stirred at 0° C. for 30 min and then pyridine (90.0 mL) in THF (108 mL) was added and the resulting mixture was stirred at 0° C. for 1 h and at 20° C. for 5 h. The reaction mixture was quenched by the addition of water and extracted with ethyl acetate, dried over anhydrous sodium sulfate, filtered, and concentrated under reduced pressure to give a residue, which was purified by column chromatography (SiO₂, petroleum ether:ethyl acetate=100:1 to 20:1) to afford diethyl 2-(1,1,1-trifluoropropan-2-ylidene)malonate as a yellow oil. ¹H NMR (400 MHz, CDCl₃) δ 4.29 (q, J=7.2 Hz, 4H), 2.22 (s, 3H), 1.33-1.29 (m, 6H).

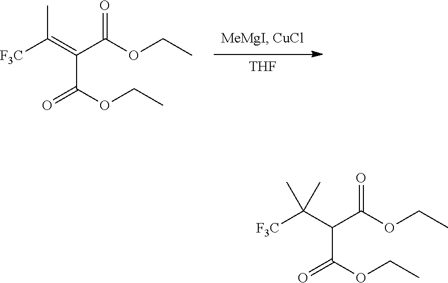

A mixture of CuCl (786 mg, 7.94 mmol, 190 μL, 0.0500 eq) and MeMgI (3 M, 78.7 mL, 1.50 eq) was stirred at 0° C. and a solution of diethyl 2-(1,1,1-trifluoropropan-2-ylidene)malonate (40.0 g, 157 mmol, 1.00 eq) in THF (400 mL) and dichloromethane (40.0 mL) was added. The reaction mixture was stirred at 0° C. for 30 min, and further stirred at 20° C. for 30 min. The reaction mixture was quenched by the addition of HCl (1M) and extracted with ethyl acetate, dried over anhydrous sodium sulfate, filtered, and concentrated under reduced pressure to afford diethyl 2-(1,1,1-trifluoro-2-methylpropan-2-yl)malonate (30.0 g, 111 mmol, 70.5% yield) as a yellow oil. ¹H NMR (400 MHz, CDCl₃) δ 4.29 (q, J=7.2 Hz, 4H), 2.22 (s, 3H), 1.33-1.29 (m, 6H). LCMS [M+Na]⁺=293.1 m/z.

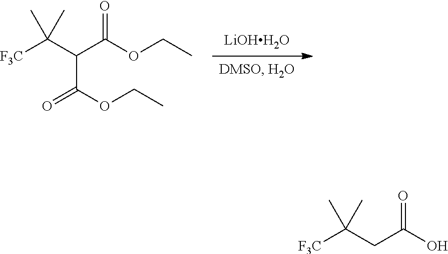

To a solution of diethyl 2-(1,1,1-trifluoro-2-methylpropan-2-yl)malonate (30.0 g, 111 mmol, 1.00 eq) in DMSO (300 mL) and water (3.00 mL) was added LiOH·H₂O (23.3 g, 555 mmol, 5.00 eq). The reaction mixture was stirred at 90° C. for 1 h. The reaction mixture was diluted with water and the pH was adjusted to 5 with HCl (1M) and an extraction was performed with ethyl acetate, organic layer was washed with water, dried over anhydrous sodium sulfate, filtered, and concentrated under reduced pressure to afford 4,4,4-trifluoro-3,3-dimethylbutanoic acid (16.5 g, 97.0 mmol, 87.3% yield) as a yellow oil. ¹H NMR (400 MHz, DMSO-d₆) δ 12.6-12.4 (m, 1H), 2.39 (s, 1H), 1.22 (s, 1H).

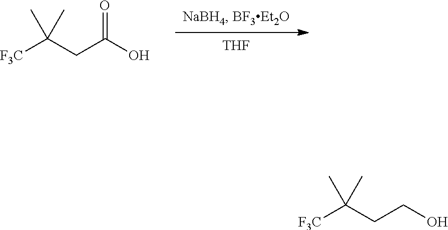

To a solution of 4,4,4-trifluoro-3,3-dimethylbutanoic acid (14.0 g, 82.3 mmol, 1.00 eq) in THF (140 mL) was added NaBH₄ (3.43 g, 90.7 mmol, 1.10 eq) at 0° C. followed by BF₃·Et₂O (11.7 g, 82.3 mmol, 10.1 mL, 1.00 eq). The reaction mixture was stirred at 20° C. for 2 h. The reaction mixture was quenched by saturated aqueous solution of NH₄Cl and extracted with ethyl acetate, dried over anhydrous sodium sulfate, filtered, and concentrated under reduced pressure to afford 4,4,4-trifluoro-3,3-dimethylbutan-1-ol (12.0 g, 76.9 mmol, 93.3% yield) as a yellow oil.

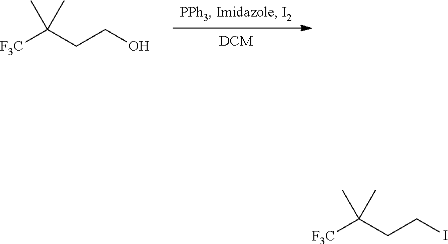

To a solution of 4,4,4-trifluoro-3,3-dimethylbutan-1-ol (12.0 g, 76.9 mmol, 1.00 eq) in dichloromethane (300 mL) was added PPh₃ (32.3 g, 123 mmol, 1.60 eq) and Imidazole (6.80 g, 99.9 mmol, 1.30 eq) and cooled to 0° C., then was added I₂ (31.2 g, 123 mmol, 24.8 mL, 1.60 eq). The reaction mixture was stirred at 20° C. for 5 h. The reaction mixture was quenched by saturated aqueous solution of NaHCO₃, then extracted with dichloromethane and washed with saturated aqueous solution of NaHCO₃ and water, dried over anhydrous sodium sulfate, filtered, and concentrated under reduced pressure to give a residue, which was purified by re-crystallization from petroleum ether at 20° C. to afford 1,1,1-trifluoro-4-iodo-2,2-dimethylbutane (7.00 g, 26.3 mmol, 34.2% yield) as a colorless oil. ¹H NMR (400 MHz, CDCl₃) δ 3.22-3.17 (m, 2H), 2.21-2.17 (m, 2H), 1.15 (s, 6H).

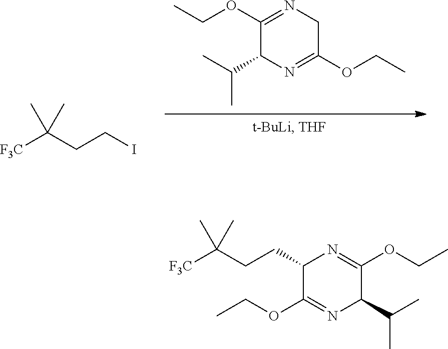

To a solution of (R)-3,6-diethoxy-2-isopropyl-2,5-dihydropyrazine (6.70 g, 31.6 mmol, 1.20 eq) in THF (70.0 mL) was added t-BuLi (1.3 M, 24.3 mL, 1.20 eq) at −78° C. and stirred for 1 h, then a solution of 1,1,1-trifluoro-4-iodo-2,2-dimethylbutane (7.00 g, 26.3 mmol, 1.00 eq) in THF (50.0 mL) was added. The resulting mixture was stirred at 20° C. for 2 h. The reaction mixture was quenched by saturated aqueous solution of NH₄Cl and extracted with ethyl acetate, dried over anhydrous sodium sulfate, filtered, and concentrated under reduced pressure to give a residue, which was purified by column chromatography (SiO₂, petroleum ether:ethyl acetate=100:1 to 10:1) to afford (2R,5S)-3,6-diethoxy-2-isopropyl-5-(4,4,4-trifluoro-3,3-dimethylbutyl)-2,5-dihydropyrazine (3.80 g, 10.8 mmol, 41.2% yield) a colorless oil. ¹H NMR (400 MHz, MeOD) 5.18-4.10 (m, 4H), 4.04-4.00 (m, 1H), 3.36 (t, J=3.6 Hz, 1H), 2.29-2.25 (m, 1H), 1.88-1.77 (m, 2H), 1.64-1.52 (m, 1H), 1.43-1.39 (m, 1H), 1.29 (t, J=6.8 Hz, 6H), 1.08 (s, 6H), 1.04 (d, J=6.8 Hz, 3H), 0.72 (d, J=6.8 Hz, 3H). LCMS [M+H]⁺=351.4 m/z.

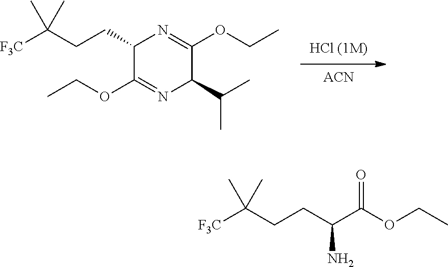 [SMILES: CCOC1=N[C@H](C(C)C)C(OCC)=N[C@H]1CCC(C)(C)C(F)(F)F ; "ACN" ; "HCl (1M)" ; CCOC(=O)[C@@H](N)CCC(C)(C)C(F)(F)F]

To a solution of (2R,5S)-3,6-diethoxy-2-isopropyl-5-(4,4,4-trifluoro-3,3-dimethylbutyl)-2,5-dihydropyrazine (3.80 g, 10.8 mmol, 1.00 eq) in acetonitrile (40.0 mL) was added HCl (1M) (1 M, 35.0 mL, 3.23 eq). The reaction mixture was stirred at 20° C. for 2 h. The reaction mixture was quenched by saturated aqueous solution of NaHCO₃ and extracted with ethyl acetate, dried over anhydrous sodium sulfate, filtered, and concentrated under reduced pressure to afford ethyl (S)-2-amino-6,6,6-trifluoro-5,5-dimethylhexanoate (2.50 g, 10.4 mmol, 95.56% yield) as a yellow oil.

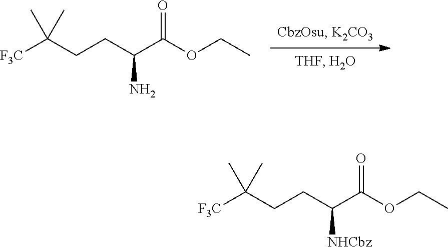

To a solution of ethyl (S)-2-amino-6,6,6-trifluoro-5,5-dimethylhexanoate (2.50 g, 10.4 mmol, 1.00 eq), CbzOSu (2.84 g, 11.4 mmol, 1.10 eq) in THF (40.0 mL) and water (40.0 mL) was added K₂CO₃ (4.30 g, 31.1 mmol, 3.00 eq). The reaction mixture was stirred at 20° C. for 1.5 h. The reaction mixture was diluted with water and extracted with ethyl acetate, dried over anhydrous sodium sulfate, filtered, and concentrated under reduced pressure to give a residue, which was purified by column chromatography (SiO₂, petroleum ether:ethyl acetate=100:1 to 3:1) to afford ethyl (S)-2-(((benzyloxy)carbonyl)amino)-6,6,6-trifluoro-5,5-dimethylhexanoate (2.10 g, 5.59 mmol, 53.9% yield) as a colorless oil. LCMS [M+H]⁺=376.2 m/z.

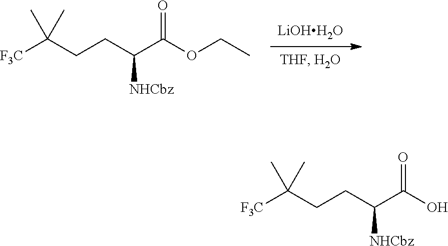

To a solution of ethyl (S)-2-(((benzyloxy)carbonyl)amino)-6,6,6-trifluoro-5,5-dimethylhexanoate (2.00 g, 5.33 mmol, 1.00 eq) in THF (20.0 mL) and water (3.00 mL) was added LiOH·H2O (440 mg, 18.4 mmol, 3.45 eq). The reaction mixture was stirred at 20° C. for 2 h. The pH was adjusted to 4 with HCl (1M), then extracted with ethyl acetate, dried over sodium sulfate, filtered, and concentrated under reduced pressure to afford (S)-2-(((benzyloxy)carbonyl)amino)-6,6,6-trifluoro-5,5-dimethylhexanoic acid (1.60 g, 4.61 mmol, 86.4% yield) as a colorless oil.

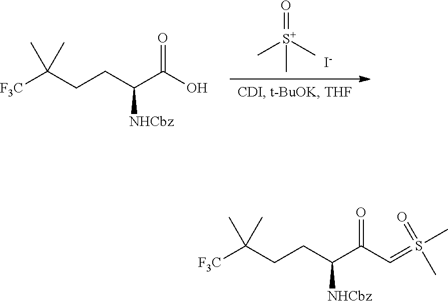

The preparation of mixture A: trimethylsulfoxonium iodide (1.62 g, 7.37 mmol, 1.60 eq) was added to a solution of t-BuOK (1 M, 6.91 mL, 1.50 eq) in THF (30.0 mL) and the reaction mixture was stirred at 20° C. for 1 h under N₂. After that time, the solution was cooled to 0° C. to obtain the mixture A.

The preparation of mixture B: To a solution of (S)-2-(((benzyloxy)carbonyl)amino)-6,6,6-trifluoro-5,5-dimethylhexanoic acid (1.60 g, 4.61 mmol, 1.00 eq) in THF (20.0 mL) was added CDI (896 mg, 5.53 mmol, 1.20 eq) at 0° C. The reaction mixture was stirred at 0° C. for 1 h. The suspension was then filtered, and the filtrate was cooled to 0° C. to obtain the mixture B.

The mixture B was added to the mixture A at 0° C. The resulting mixture was stirred at 20° C. for 2 h. The reaction mixture was quenched by the addition of water at room temperature, and extracted with ethyl acetate. The combined organic layers were dried over anhydrous sodium sulfate, filtered, and concentrated under reduced pressure to give a residue, which was purified by reversed-phase HPLC (0.1% FA condition) to afford benzyl (S)-(1-(dimethyl(oxo)-λ6-sulfaneylidene)-7,7,7-trifluoro-6,6-dimethyl-2-oxoheptan-3-yl)carbamate (1.20 g, 2.85 mmol, 61.8% yield) as a light yellow oil. ¹H NMR (400 MHz, CDCl₃) δ 7.43-7.29 (m, 5H), 5.62 (d, J=8.0 Hz, 1H), 5.14-5.07 (m, 2H), 4.51 (s, 1H), 4.17-4.12 (m, 1H), 3.39 (s, 6H), 1.89-1.84 (m, 1H), 1.64-1.45 (m, 3H), 1.06 (s, 6H). LCMS [M+H]⁺=422.2 m/z.

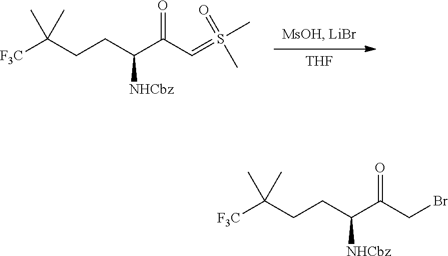

To a solution of benzyl (S)-(1-(dimethyl(oxo)-λ6-sulfaneylidene)-7,7,7-trifluoro-6,6-dimethyl-2-oxoheptan-3-yl)carbamate (150 mg, 356 μmol, 1.00 eq) in THF (5.00 mL) was added LiBr (46.4 mg, 534 μmol, 13.4 μL, 1.50 eq) and MsOH (51.3 mg, 534 μmol, 38.1 μL, 1.50 eq) at 0° C. The reaction mixture was stirred at 45° C. for 1 h. The reaction mixture was diluted with water and extracted with ethyl acetate, dried over anhydrous sodium sulfate, filtered, and concentrated under reduced pressure to afford benzyl (S)-(1-bromo-7,7,7-trifluoro-6,6-dimethyl-2-oxoheptan-3-yl)carbamate (200 mg, crude) as a yellow solid.

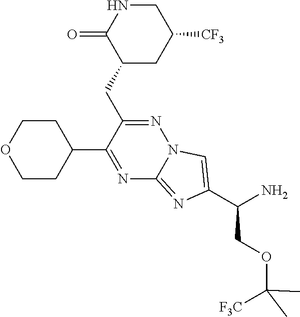 [SMILES: CC(C)(OC[C@H](N)c1cn2nc(C[C@H]3C[C@@H](C(F)(F)F)CNC3=O)c(C3CCOCC3)nc2n1)C(F)(F)F]

(3R,5R)-3-((6-((S)-1-amino-5,5,5-trifluoro-4,4-dimethylpentyl)-3-(tetrahydro-2H-pyran-4-yl)imidazo[1,2-b][1,2,4]triazin-2-yl)methyl)-5-(trifluoromethyl)piperidin-2-one was synthesized according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one and benzyl (S)-(1-bromo-7,7,7-trifluoro-6,6-dimethyl-2-oxoheptan-3-yl)carbamate, and General Procedure 4, employing benzyl ((S)-5,5,5-trifluoro-4,4-dimethyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)pentyl)carbamate.

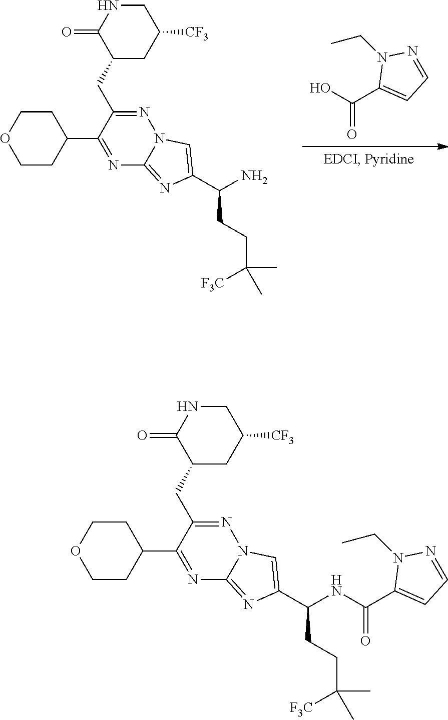

1-Ethyl-N-((1S)-5,5,5-trifluoro-4,4-dimethyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)pentyl)-1H-pyrazole-5-carboxamide was synthesized following the General Procedure 6 with ((3R,5R)-3-((6-((S)-1-amino-5,5,5-trifluoro-4,4-dimethylpentyl)-3-(tetrahydro-2H-pyran-4-yl)imidazo[1,2-b][1,2,4]triazin-2-yl)methyl)-5-(trifluoromethyl)piperidin-2-one (50.0 mg, 90.8 μmol, 1.00 eq), 1-ethyl-1H-pyrazole-5-carboxylic acid (19.1 mg, 136 μmol, 1.50 eq) and was purified by prep-TLC (SiO₂, dichloromethane:methanol) to afford 1-ethyl-N-((1S)-5,5,5-trifluoro-4,4-dimethyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)pentyl)-1H-pyrazole-5-carboxamide (45.0 mg, 66.9 mol, 73.7% yield) as a white solid. LCMS [M+H]⁺=673.4 nm/z.

Compound 160 was isolated as the first eluting, single stereoisomer by chiral SFC purification (column: Daicel Chiralpak AD (250 mm×30 mm, 10 μm); mobile phase: [CO₂-i-PrOH]; B %:30%, isocratic elution mode); (21.4 mg, 29.0 μmol, 55.7% yield) and was obtained as a yellow solid. LCMS [M+H]⁺=673.3 m/z.

Compound 217 was isolated as the second eluting, single stereoisomer by chiral SFC purification (column: Daicel Chiralpak AD (250 mm×30 mm, 10 μm); mobile phase: [CO₂-i-PrOH]; B %:30%, isocratic elution mode); (33.2 mg, 48.7 μmol, 93.5% yield) and was obtained as a yellow solid. LCMS [M+H]⁺=673.4 m/z.

### Example 157: General Procedure 14

### General Procedure 14

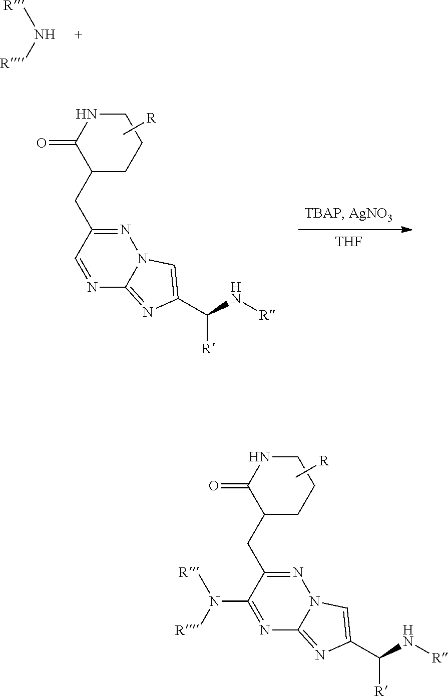

To a solution of imidazo[1,2-b][1,2,4]triazine derivative (1.00 eq) and amine (10.0-20.0 eq) in THF (0.05-0.2 M) at 0° C. was added AgNO₃ (2.00 eq) and tetrabutylammonium permanganate (TBAP, 2.00 eq). The reaction was stirred at 25° C. until completion. The reaction mixture was then filtered and concentrated under reduced pressure. Purification by column chromatography, prep-TLC, and/or prep-HPLC, optionally followed by basic workup (for freebase), afforded the corresponding 7-(amino)imidazo[1,2-b][1,2,4]triazine derivative.

### Example 158. General Procedure 15

### General Procedure 15

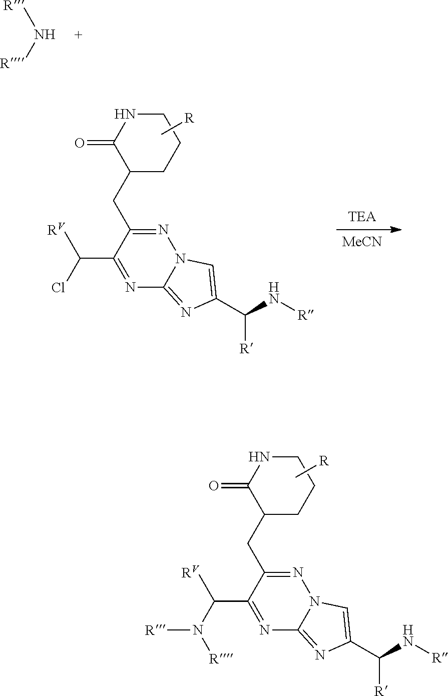

To a solution of amine (1.20-10.0 eq) and benzylic chloride (1.00 eq) in MeCN or DMF or DCM was optionally added TEA or DIEA (2.00-3.00 eq). The reaction mixture was stirred at 25-50° C. for 1-8 h. The reaction mixture was then diluted with H₂O and extracted with 3×EtOAc. The combined organic layers were dried over Na₂SO₄, filtered, and concentrated. Crude product was purified by prep-TLC, prep-HPLC, and/or SFC to afford the desired benzylic amine derivative.

### Example 159. Preparation of N—((S)-(3-(2-oxabicyclo[3.1.1]heptan-5-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 250)

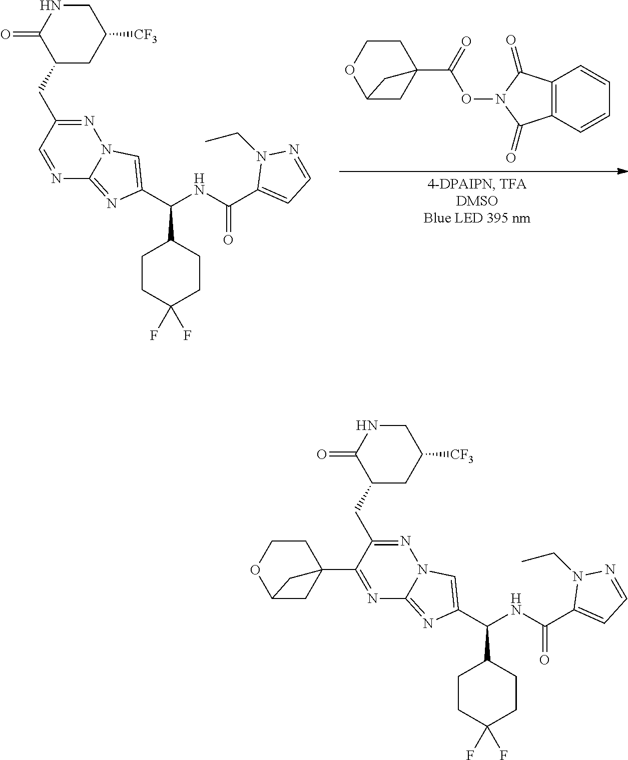 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C1c2ccccc2C(=O)N1OC(=O)C12CCOC(C1)C2 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C34CCOC(C3)C4)nc2n1)C1CCC(F)(F)CC1]

N—((S)-(3-(2-oxabicyclo[3.1.1]heptan-5-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 2-oxabicyclo[3.1.1]heptane-5-carboxylate (36.3 mg, 126 μmol, 1.20 eq) and was purified by Prep-TLC (SiO₂, Dichloromethane:Methanol=100:1 to 10:1) to afford N—((S)-(3-(2-oxabicyclo[3.1.1]heptan-5-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (27.7 mg, 40.0 μmol, 37.9% yield, 95.9% purity) as a yellow solid.

### Example 160. N-((1S)-(4,4-difluorocyclohexyl)(2-(((3R,5R)-2-oxo-5-(trifluoromethyl)piperidin-3-yl)methyl)-3-(1-(tetrahydro-2H-pyran-4-yl)ethyl)imidazo[1,2-b][1,2,4]triazin-6-yl)methyl)-1-ethyl-1H-pyrazole-5-carboxamide (Compound 251 and 252)

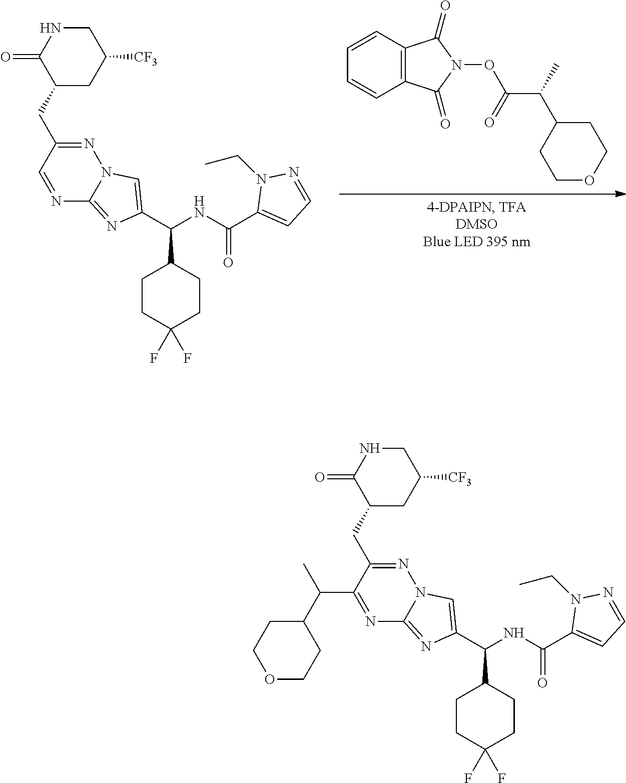 [SMILES: CCn1nccc1C(=O)N[C@H](c1cn2nc(C[C@H]3C[C@@H](C(F)(F)F)CNC3=O)cnc2n1)C1CCC(F)(F)CC1 ; C[C@@H](C(=O)ON1C(=O)c2ccccc2C1=O)C1CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)C3CCOCC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(1-(tetrahydro-2H-pyran-4-yl)ethyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (149 mg, 492 μmol, 2.00 eq), 1,3-dioxoisoindolin-2-yl (R)-2-(tetrahydro-2H-pyran-4-yl)propanoate (140 mg, 246 μmol, 1.00 eq) and was purified by Prep-HPLC (column: Phenomenex luna C18 150 mm×25 mm, 10 um; mobile phase: [water (FA)-ACN], gradient: 50%-70% B over 10 min) to afford N-((1S)-(4,4-difluorocyclohexyl)(2-(((3R,5R)-2-oxo-5-(trifluoromethyl)piperidin-3-yl)methyl)-3-(1-(tetrahydro-2H-pyran-4-yl)ethyl)imidazo[1,2-b][1,2,4]triazin-6-yl)methyl)-1-ethyl-1H-pyrazole-5-carboxamide (140 mg, 205 μmol, 83.5% yield) as a yellow solid.

Compound 251 was isolated as the first eluting, single stereoisomer by chiral SFC purification (column: Daicel Chiralcel OD-H (250 mm×30 mm, 5 um); mobile phase: [CO₂-i-PrOH]; B %: 30%, isocratic elution mode); (34.7 mg, 49.5 μmol, 24.1% yield, 97.0% purity) and was obtained as a white solid.

Compound 252 was isolated as the second eluting, single stereoisomer by chiral SFC purification (column: Daicel Chiralcel OD-H (250 mm×30 mm, 5 um); mobile phase: [CO₂-i-PrOH]; B %: 30%, isocratic elution mode); (38.04 mg, 53.7 μmol, 26.1% yield, 96.1% purity) and was obtained as a white solid.

### Example 161. Preparation of N—((S)-(3-(3-oxabicyclo[3.1.1]heptan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 253)

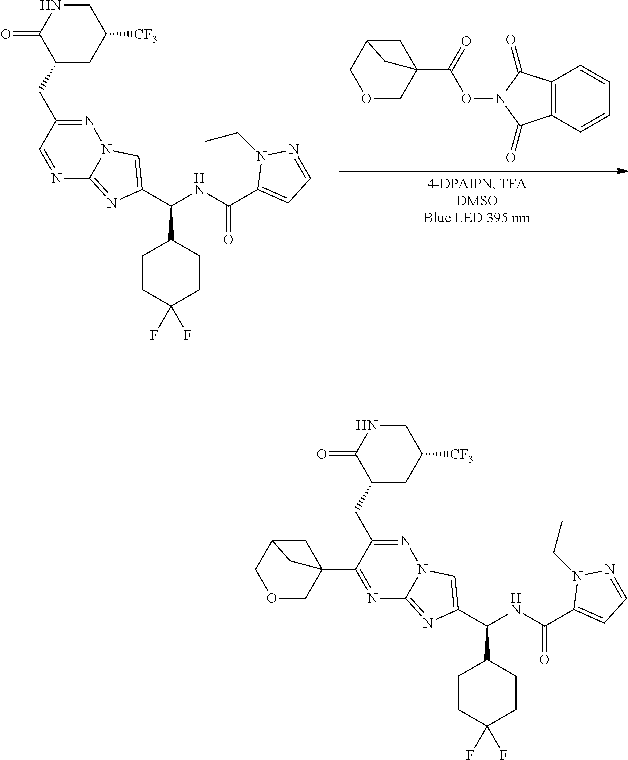 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C1c2ccccc2C(=O)N1OC(=O)C12COCC(C1)C2 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C34COCC(C3)C4)nc2n1)C1CCC(F)(F)CC1]

N—((S)-(3-(3-oxabicyclo[3.1.1]heptan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 3-oxabicyclo[3.1.1]heptane-1-carboxylate (75.7 mg, 263 μmol, 2.50 eq) and was purified by Prep-HPLC (column: Phenomenex luna C18 150 mm×25 mm, 10 um; mobile phase: [water (FA)-ACN]; gradient: 40%-70% B over 10 min) to afford N—((S)-(3-(3-oxabicyclo[3.1.1]heptan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (45.43 mg, 66.9 μmol, 63.4% yield, 97.9% purity) as a white solid.

### Example 162. Preparation of N-((1S)-(3-(6-oxabicyclo[3.2.1]octan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 254)

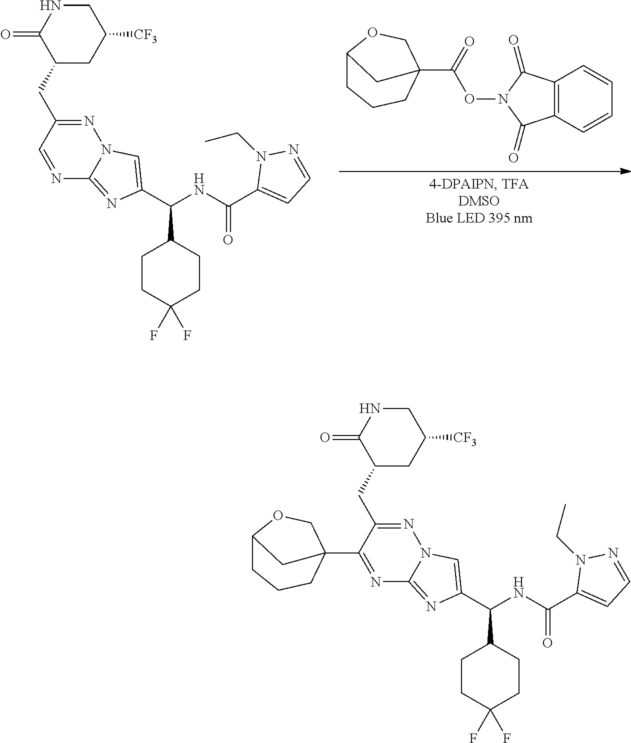 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C1c2ccccc2C(=O)N1OC(=O)C12CCCC(C1)OC2 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C34CCCC(C3)OC4)nc2n1)C1CCC(F)(F)CC1]

N-((1S)-(3-(6-oxabicyclo[3.2.1]octan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (80.0 mg, 140 μmol, 1.00 eq), 1,3-dioxoisoindolin-2-yl 6-oxabicyclo[3.2.1]octane-1-carboxylate (84.7 mg, 281 μmol, 2.00 eq) and was purified by Prep-HPLC (FA condition: column: Phenomenex luna C18 150 mm×25 mm, 10 um; mobile phase: [water (FA)-ACN]; gradient: 43%-73% B over 10 min) to afford N-((1S)-(3-(6-oxabicyclo[3.2.1]octan-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (41.02 mg, 58.9 μmol, 41.9% yield, 97.6% purity) as a white solid.

### Example 163. Preparation of N—((S)-(3-(2-oxabicyclo[2.1.1]hexan-4-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 255)

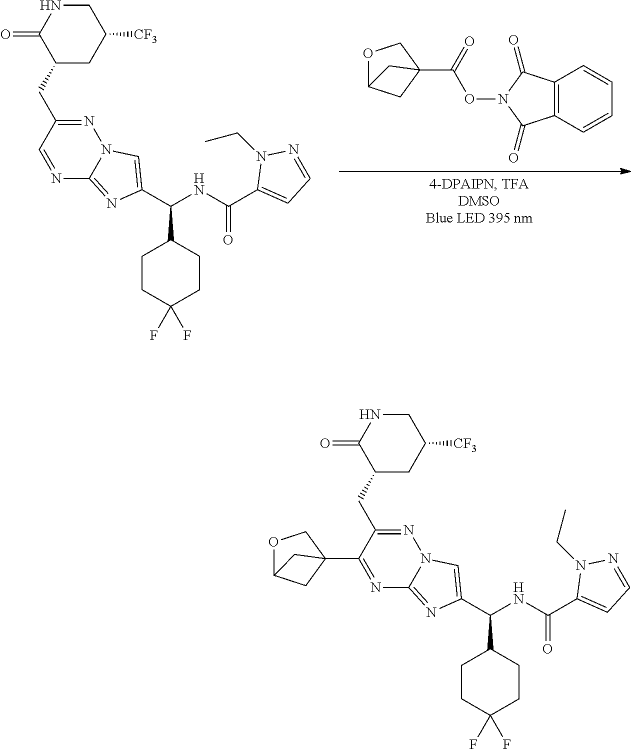 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C1c2ccccc2C(=O)N1OC(=O)C12COC(C1)C2 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C34COC(C3)C4)nc2n1)C1CCC(F)(F)CC1]

N—((S)-(3-(2-oxabicyclo[2.1.1]hexan-4-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 2-oxabicyclo[2.1.1]hexane-4-carboxylate (72.0 mg, 263 μmol, 2.50 eq) and was purified by Prep-HPLC (column: Phenomenex luna C18 150 mm×25 mm, 10 um; mobile phase: [water (FA)-ACN]; gradient: 40%-60% B over 10 min) to afford N—((S)-(3-(2-oxabicyclo[2.1.1]hexan-4-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (25.47 mg, 37.4 μmol, 35.4% yield, 95.6% purity) as a white solid.

### Example 164. Preparation of N-((1S)-(4,4-difluorocyclohexyl)(3-(6,6-dimethyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 256 and 258)

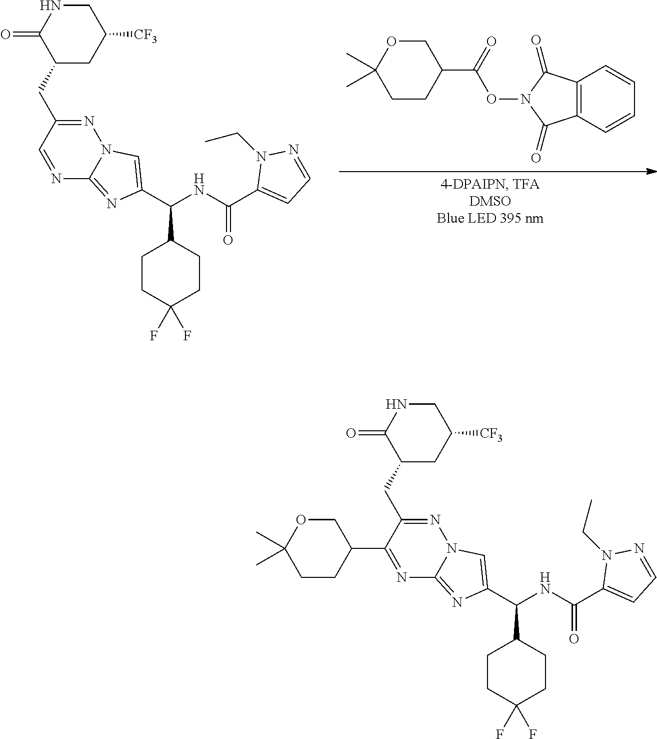 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC1(C)CCC(C(=O)ON2C(=O)c3ccccc3C2=O)CO1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCC(C)(C)OC3)nc2n1)C1CCC(F)(F)CC1]

N-((1S)-(4,4-difluorocyclohexyl)(3-(6,6-dimethyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1,3-dioxoisoindolin-2-yl 6,6-dimethyltetrahydro-2H-pyran-3-carboxylate (128 mg, 422 μmol, 2.00 eq) and was purified by Prep-HPLC (column: Phenomenex luna C18 150 mm×25 mm, 10 um; mobile phase: [water (FA)-ACN]; gradient: 47%-77% B over 10 min) to afford N-((1S)-(4,4-difluorocyclohexyl)(3-(6,6-dimethyltetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 172 μmol, 81.7% yield, 97.9% purity) as a white solid.

Compound 256 was isolated as the first eluting, single stereoisomer by chiral SFC purification (column: Daicel Chiralcel OD-H (250 mm×30 mm, 5 μm); mobile phase: [CO₂-i-PrOH]; B %:25%, isocratic elution mode); (25.97 mg, 36.7 μmol, 20.8% yield, 96.2% purity) and was obtained as a white solid.

Compound 258 was isolated as the second eluting, single stereoisomer by chiral SFC purification (column: Daicel Chiralcel OD-H (250 mm×30 mm, 5 μm); mobile phase: [CO₂-i-PrOH]; B %:25%, isocratic elution mode); (29.04 mg, 41.4 μmol, 23.5% yield, 97.1% purity) and was obtained as a white solid.

### Example 165. Preparation of N-((1S)-(4,4-difluorocyclohexyl)(3-(4,4-difluorotetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 257 and 259)

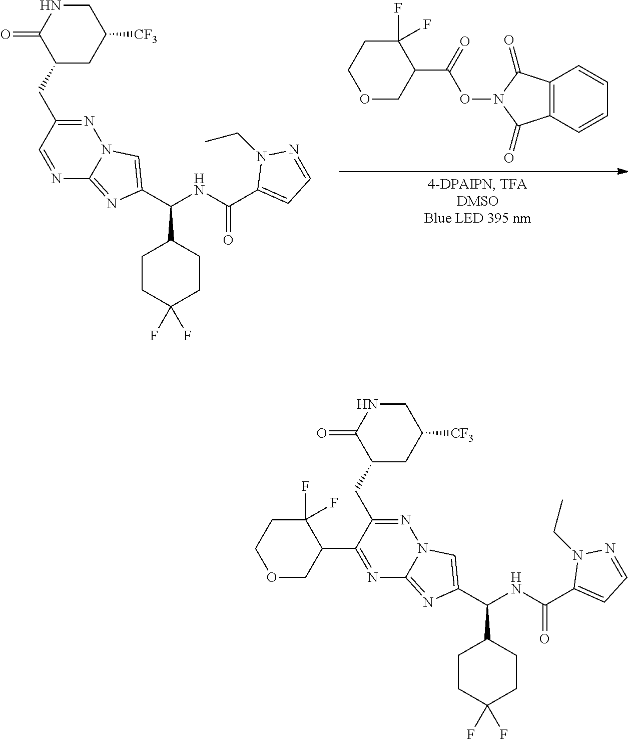 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1COCCC1(F)F ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3COCCC3(F)F)nc2n1)C1CCC(F)(F)CC1]

N-((1S)-(4,4-difluorocyclohexyl)(3-(4,4-difluorotetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (60.0 mg, 105 μmol, 1.00 eq), 1,3-dioxoisoindolin-2-yl 4,4-difluorotetrahydro-2H-pyran-3-carboxylate (65.6 mg, 211 μmol, 2.00 eq) and was purified by Prep-HPLC (column: Phenomenex luna C18 150 mm×25 mm, 10 um; mobile phase: [water (FA)-ACN]; gradient: 43%-63% B over 10 min) to afford N-((1S)-(4,4-difluorocyclohexyl)(3-(4,4-difluorotetrahydro-2H-pyran-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (15.0 mg, 21.7 μmol, 20.6% yield) as a white solid.

Compound 257 was isolated as the first eluting, single stereoisomer by Prep-HPLC (column: Phenomenex luna C18 150 mm×25 mm, 10 um; mobile phase: [water (FA)-ACN]; gradient: 43%-63% B over 10 min); (4.39 mg, 6.16 μmol, 28.2% yield, 96.6% purity) and was obtained as a white solid.

Compound 259 was isolated as the second eluting, single stereoisomer by Prep-HPLC (column: Phenomenex luna C18 150 mm×25 mm, 10 um; mobile phase: [water (FA)-ACN]; gradient: 43%-63% B over 10 min); (4.35 mg, 6.30 μmol, 28.9% yield, 99.8% purity) and was obtained as a white solid.

### Example 166. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(1,1-difluoroethyl)isoxazole-4-carboxamide (Compound 260)

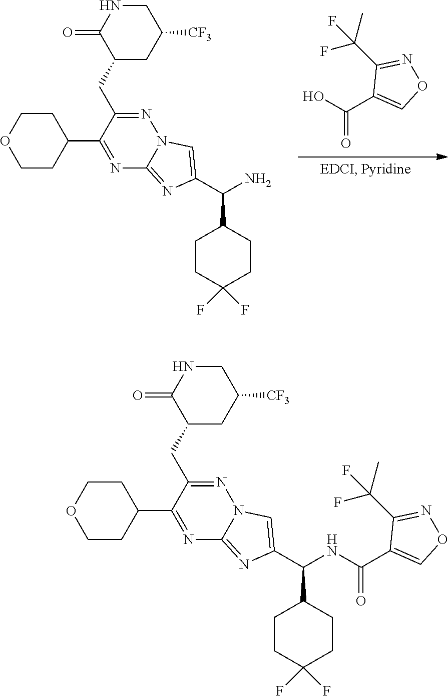

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(1,1-difluoroethyl)isoxazole-4-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 3-(1,1-difluoroethyl)isoxazole-4-carboxylic acid (20.0 mg, 113 μmol, 1.20 eq) and was purified by prep-TLC (SiO₂, dichloromethane:methanol) and further purified by Prep-SFC (column: Daicel Chiralcel OX (250 mm×30 mm, 10 um); mobile phase: [CO₂-i-PrOH]; B %:30%, isocratic elution mode) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-(1,1-difluoroethyl)isoxazole-4-carboxamide (12.07 mg, 16.4 μmol, 17.4% yield, 94.1% purity) as a yellow solid.

### Example 167. Preparation of N—((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 261)

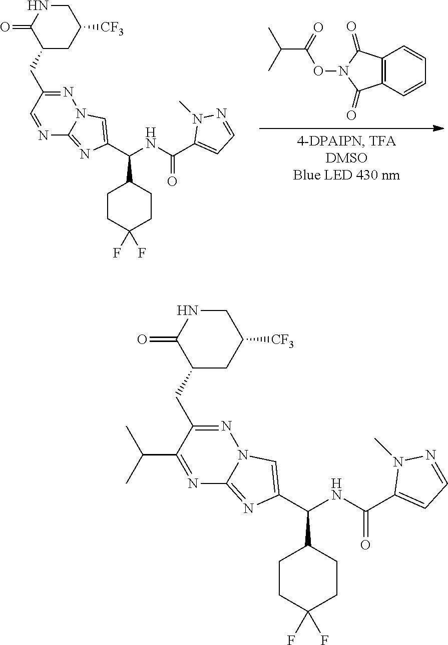 [SMILES: Cn1nccc1C(=O)N[C@H](c1cn2nc(C[C@H]3C[C@@H](C(F)(F)F)CNC3=O)cnc2n1)C1CCC(F)(F)CC1 ; CC(C)C(=O)ON1C(=O)c2ccccc2C1=O ; "4-DPAIPN, TFADMSOBlue LED 430 nm" ; CC(C)c1nc2nc([C@@H](NC(=O)c3ccnn3C)C3CCC(F)(F)CC3)cn2nc1C[C@H]1C[C@@H](C(F)(F)F)CNC1=O]

N—((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (65.0 mg, 117 μmol, 1.00 eq), 1,3-dioxoisoindolin-2-yl isobutyrate (68.0 mg, 293 μmol, 2.50 eq) and was purified by Prep-HPLC (column: Phenomenex luna C18 150 mm×25 mm, 10 um; mobile phase: [water (FA)-ACN]; gradient: 42%-62% B over 10 min) to afford N—((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (37.7 mg, 60.7 μmol, 51.7% yield, 96.0% purity) as a white solid.

### Example 168. Preparation of N—((S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 262)

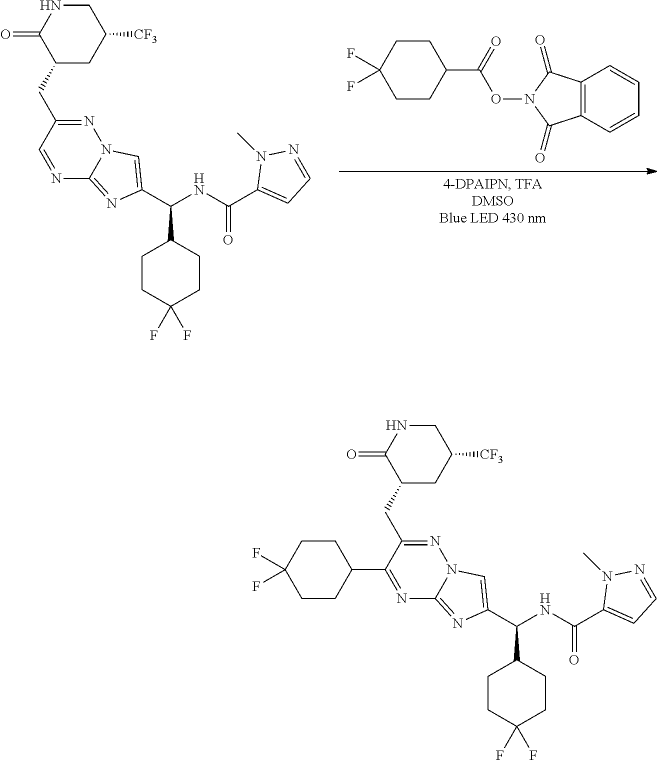 [SMILES: Cn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCC(F)(F)CC1 ; "4-DPAIPN, TFADMSOBlue LED 430 nm" ; Cn1nccc1C(=O)N[C@H](c1cn2nc(C[C@H]3C[C@@H](C(F)(F)F)CNC3=O)c(C3CCC(F)(F)CC3)nc2n1)C1CCC(F)(F)CC1]

N-((1S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (55.0 mg, 99.1 μmol, 1.00 eq), 1,3-dioxoisoindolin-2-yl 4,4-difluorocyclohexane-1-carboxylate (76.4 mg, 247 μmol, 2.50 eq) and was purified by Prep-HPLC (column: Phenomenex luna C18 150 mm×25 mm, 10 um; mobile phase: [water (FA)—ACN]; gradient: 40%-70% B over 9 min) to afford N—((S)-(4,4-difluorocyclohexyl)(3-(4,4-difluorocyclohexyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (48.1 mg, 70.2 μmol. 70.8% yield. 98.3% purity) as a yellow solid.

### Example 169. Preparation of 1-methyl-N—((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (Compound 218)

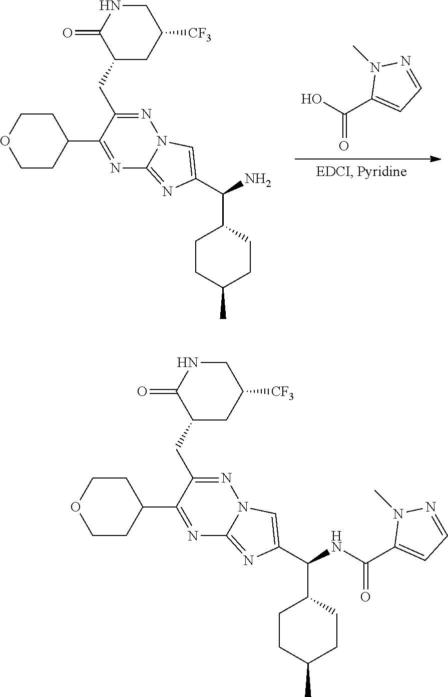

1-Methyl-N—((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide was synthesized following the General Procedure 6 with (3R,5R)-3-((6-((S)-amino((1r,4S)-4-methylcyclohexyl)methyl)-3-(tetrahydro-2H-pyran-4-yl)imidazo[1,2-b][1,2,4]triazin-2-yl)methyl)-5-(trifluoromethyl)piperidin-2-one (60.0 mg, 117 μmol, 1.00 eq), 1-methyl-1H-pyrazole-5-carboxylic acid (16.3 mg, 129 μmol, 1.10 eq) and was purified by Prep-HPLC (column: Phenomenex luna C18 150 mm×25 mm, 10 um; mobile phase: [water (FA)—ACN]; gradient: 48%-58% B over 10 min) to afford 1-methyl-N—((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (12.07 mg, 16.4 μmol, 17.4% yield, 94.1% purity) as a white solid.

### Example 170. Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide (Compound 263 and 264)

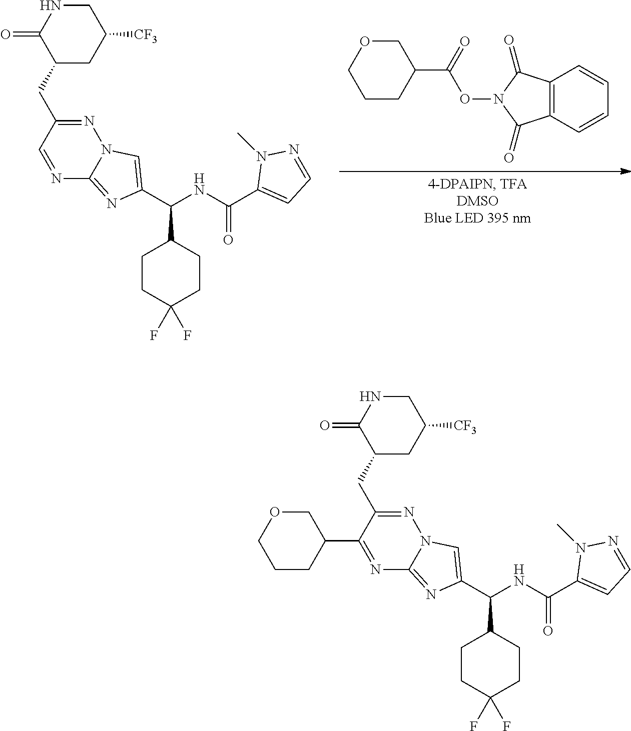 [SMILES: Cn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCCOC1 ; "4-DPAIPN, TFADMSOBlue LED 395 nm" ; Cn1nccc1C(=O)N[C@H](c1cn2nc(C[C@H]3C[C@@H](C(F)(F)F)CNC3=O)c(C3CCCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (110 mg, 198 μmol, 1.00 eq), 1,3-dioxoisoindolin-2-yl tetrahydro-2H-pyran-3-carboxylate (136 mg, 495 μmol, 2.50 eq) and was purified by Prep-HPLC (column: Phenomenex luna C18 150 mm×25 mm, 10 um; mobile phase: [water (FA)—ACN]; gradient: 46%-76% B over 9 min) to afford N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide.

Compound 263 was isolated as the first eluting, single stereoisomer by Prep-SFC (column: Daicel Chiralpak AD (250 mm×30 mm, 10 um); mobile phase: [CO₂-i-PrOH]; B %:35%, isocratic elution mode); (36.6 mg, 57.2 μmol, 28.8% yield, 99.8% purity) and was obtained as a white solid.

Compound 264 was isolated as the second eluting, single stereoisomer by Prep-SFC (column: Daicel Chiralpak AD (250 mm×30 mm, 10 um); mobile phase: [CO₂-i-PrOH]; B %:35%, isocratic elution mode); (39.65 mg, 61.90 μmol, 31.20% yield, 99.7% purity) and was obtained as a white solid.

### Example 171. Preparation of N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-4-methyl-1,2,5-oxadiazole-3-carboxamide (Compound 265 and 266)

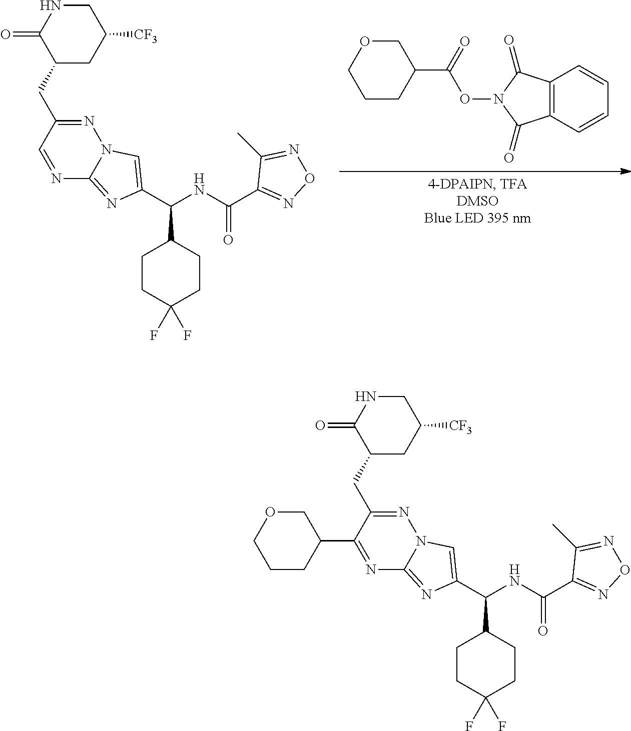 [SMILES: Cc1nonc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCCOC1 ; "4-DPAIPN, TFADMSOBlue LED 395 nm" ; Cc1nonc1C(=O)N[C@H](c1cn2nc(C[C@H]3C[C@@H](C(F)(F)F)CNC3=O)c(C3CCCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-4-methyl-1,2,5-oxadiazole-3-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (120 mg, 215 μmol, 1.00 eq), 1,3-dioxoisoindolin-2-yl tetrahydro-2H-pyran-3-carboxylate (118 mg, 431 μmol, 2.00 eq) and was purified by Prep-TLC (SiO₂, Petroleum ether:Ethyl acetate=0:1) to afford N-((1S)-(4,4-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-4-methyl-1,2,5-oxadiazole-3-carboxamide (80.0 mg, 124 μmol, 57.9% yield) as a yellow solid.

Compound was isolated as the first eluting, single stereoisomer by Prep-SFC (condition: column: Daicel Chiralcel OD-H (250 mm×30 mm, 5 um); mobile phase: [CO₂-i-PrOH]; B %: 25%, isocratic elution mode); (10.9 mg, 17.0 μmol, 10.9% yield, 99.2% purity) and was obtained as a white solid.

Compound was isolated as the second eluting, single stereoisomer by Prep-SFC (condition: column: Daicel Chiralcel OD-H (250 mm×30 mm, 5 um); mobile phase: [CO₂-i-PrOH]; B %: 25%, isocratic elution mode); (9.29 mg, 14.1 μmol, 9.02% yield, 97.1% purity) and was obtained as a white solid.

### Example 172. Preparation of N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide (Compound 267 and 268)

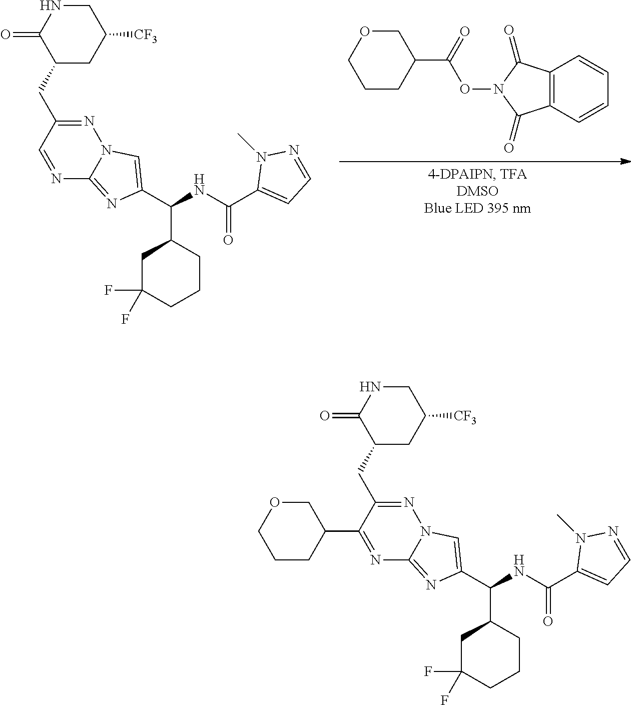 [SMILES: Cn1nccc1C(=O)N[C@H](c1cn2nc(C[C@H]3C[C@@H](C(F)(F)F)CNC3=O)cnc2n1)[C@H]1CCCC(F)(F)C1 ; O=C(ON1C(=O)c2ccccc2C1=O)C1CCCOC1 ; "4-DPAIPN, TFADMSOBlue LED 395 nm" ; Cn1nccc1C(=O)N[C@H](c1cn2nc(C[C@H]3C[C@@H](C(F)(F)F)CNC3=O)c(C3CCCOC3)nc2n1)[C@H]1CCCC(F)(F)C1]

N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide was synthesized following the General Procedure 7 with N—((S)—((S)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (200 mg, 360 μmol, 1.00 eq), 1,3-dioxoisoindolin-2-yl tetrahydro-2H-pyran-3-carboxylate (198 mg, 721 μmol, 2.00 eq) and was purified by Prep-HPLC (column: Phenomenex luna C18 150 mm×25 mm, 10 um; mobile phase: [water (FA)—ACN]; gradient: 40%-70% B over 10 min) to afford N-((1S)—((S)-3,3-difluorocyclohexyl)(2-(((3R,5R)-2-oxo-5-(trifluoromethyl)piperidin-3-yl)methyl)-3-(tetrahydro-2H-pyran-3-yl)imidazo[1,2-b][1,2,4]triazin-6-yl)methyl)-1-methyl-1H-pyrazole-5-carboxamide (150 mg, 234 μmol, 65.1% yield) as a yellow solid.

Compound 267 was isolated as the first eluting, single stereoisomer by Prep-SFC (condition: column: Daicel Chiralcel OD-H (250 mm×30 mm, 5 um); mobile phase: [CO₂-i-PrOH]; B %: 35%, isocratic elution mode); (60.8 mg, 95.0 μmol, 40.4% yield, 99.8% purity) and was obtained as a white solid.

Compound 268 was isolated as the second eluting, single stereoisomer by Prep-SFC (condition: column: Daicel Chiralcel OD-H (250 mm×30 mm, 5 um); mobile phase: [CO₂-i-PrOH]; B %: 35%, isocratic elution mode); (54.9 mg, 85.6 μmol, 36.4% yield, 99.6% purity) and was obtained as a white solid.

### Example 173. Preparation of N—((S)-(4,4-difluorocyclohexyl)(3-(4-methoxytetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 262)

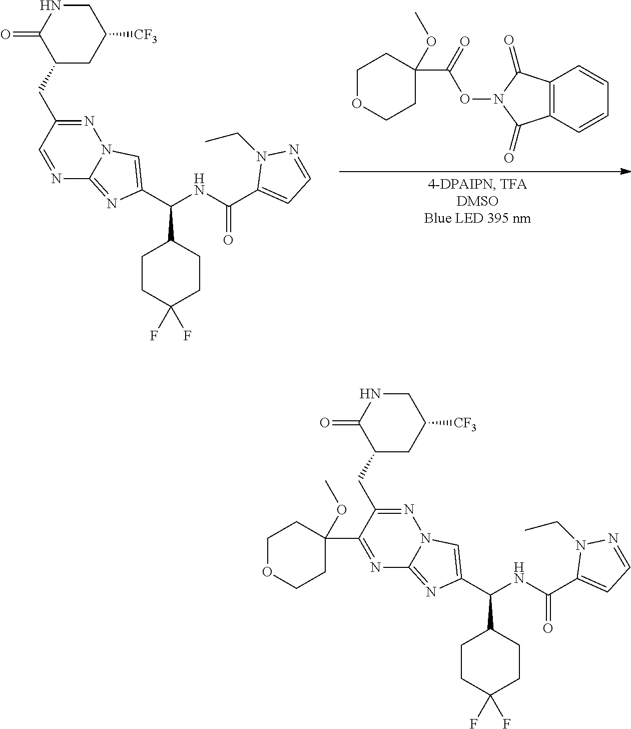 [SMILES: CCn1nccc1C(=O)N[C@H](c1cn2nc(C[C@H]3C[C@@H](C(F)(F)F)CNC3=O)cnc2n1)C1CCC(F)(F)CC1 ; COC1(C(=O)ON2C(=O)c3ccccc3C2=O)CCOCC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3(OC)CCOCC3)nc2n1)C1CCC(F)(F)CC1]

N—((S)-(4,4-difluorocyclohexyl)(3-(4-methoxytetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (65.0 mg, 114 μmol, 1.00 eq), 1,3-dioxoisoindolin-2-yl 4-methoxytetrahydro-2H-pyran-4-carboxylate (69.8 mg, 228 μmol, 2.00 eq) and was purified by Prep-HPLC (FA condition: column: Phenomenex luna C18 150 mm×25 mm, 10 um; mobile phase: [water (FA)—ACN]; gradient: 43%-63% B over 10 min) to afford N—((S)-(4,4-difluorocyclohexyl)(3-(4-methoxytetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (12.84 mg, 17.8 μmol, 15.6% yield, 95.1% purity) as a yellow solid.

### Example 174. Preparation of N—((S)-(3-(4,4-difluoro-1-methoxycyclohexyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 270)

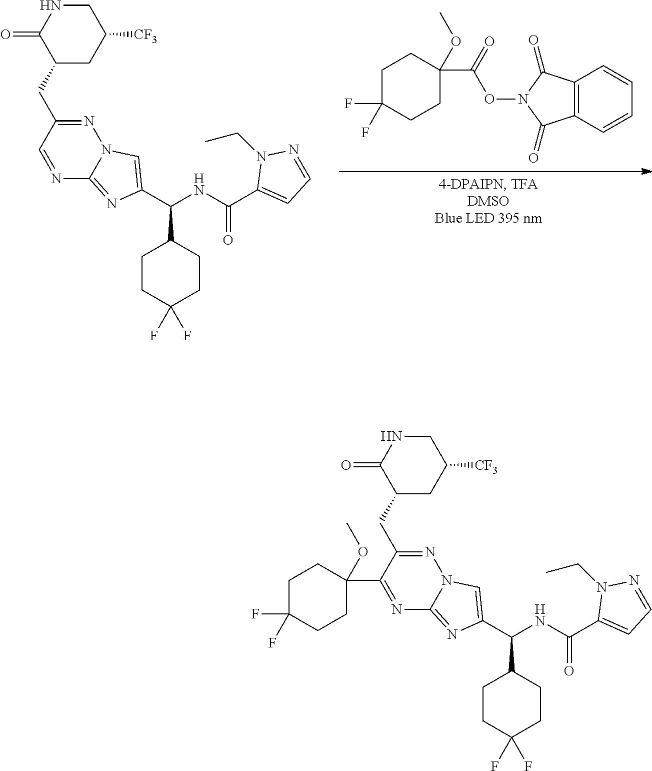 [SMILES: CCn1nccc1C(=O)N[C@H](c1cn2nc(C[C@H]3C[C@@H](C(F)(F)F)CNC3=O)cnc2n1)C1CCC(F)(F)CC1 ; COC1(C(=O)ON2C(=O)c3ccccc3C2=O)CCC(F)(F)CC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3(OC)CCC(F)(F)CC3)nc2n1)C1CCC(F)(F)CC1]

N—((S)-(3-(4,4-difluoro-1-methoxycyclohexyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 4,4-difluoro-1-methoxycyclohexane-1-carboxylate (83.5 mg, 246 μmol, 2.00 eq) and was purified by Prep-HPLC (FA condition: column: Phenomenex luna C18 150 mm×25 mm, 10 um; mobile phase: [water (FA)—ACN]; gradient: 56%-76% B over 10 min) to afford N—((S)-(3-(4,4-difluoro-1-methoxycyclohexyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (30.12 mg, 41.5 μmol, 33.7% yield, 98.9% purity) as a yellow solid.

### Example 175. Preparation of N-((1S)-(4,4-difluorocyclohexyl)(3-(3,3-difluorocyclohexyl)-2-(((3R,5R)-2-oxo-5-(trifluoromethyl)piperidin-3-yl)methyl)imidazo[1,2-b][1,2,4]triazin-6-1)methyl)-1-ethyl-H-pyrazole-5-carboxamide (Compound 271 and 272)

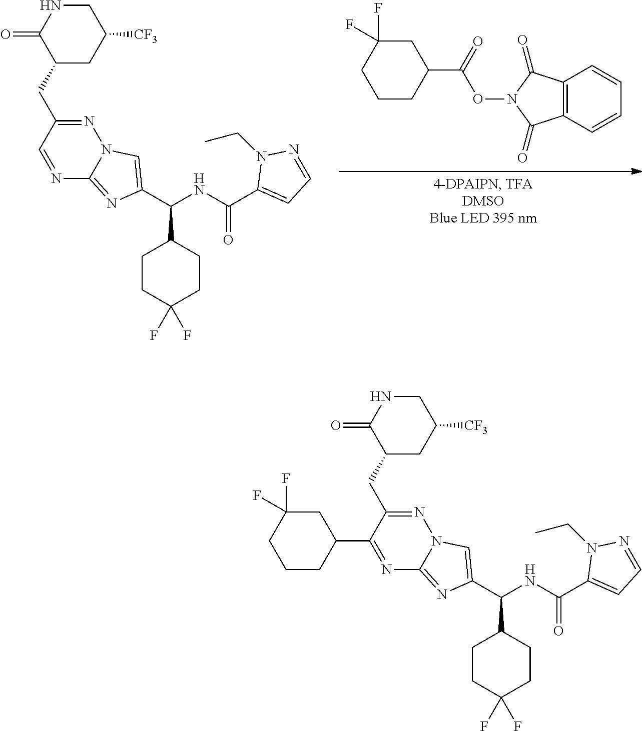 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCCC(F)(F)C1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCCC(F)(F)C3)nc2n1)C1CCC(F)(F)CC1]

N-((1S)-(4,4-difluorocyclohexyl)(3-(3,3-difluorocyclohexyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1,3-dioxoisoindolin-2-yl 3,3-difluorocyclohexane-1-carboxylate (163 mg, 527 μmol, 2.50 eq) and was purified by Prep-HPLC (column: Phenomenex luna C18 150 mm×25 mm, 10 um; mobile phase: [water (FA)—ACN]; gradient: 47%-77% B over 10 min) to afford N-((1S)-(4,4-difluorocyclohexyl)(3-(3,3-difluorocyclohexyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (80.0 mg, 116 μmol, 55.2% yield) as a white solid.

Compound 271 was isolated as the first eluting, single stereoisomer by Prep-SFC (column: Daicel Chiralpak IK (250 mm×30 mm, 10 um); mobile phase: [CO₂-i-PrOH]; B %: 35%, isocratic elution mode); (30.32 mg, 43.1 μmol, 36.9% yield, 97.6% purity) and was obtained as a white solid.

Compound 272 was isolated as the second eluting, single stereoisomer by Prep-SFC (column: Daicel Chiralpak IK (250 mm×30 mm, 10 um); mobile phase: [CO₂-i-PrOH]; B %: 35%, isocratic elution mode); (29.9 mg, 42.4 μmol, 36.4% yield, 97.5% purity) and was obtained as a white solid.

### Example 176. Preparation of N—((S)-(4,4-difluorocyclohexyl)(3-(1,1-difluoroethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 273)

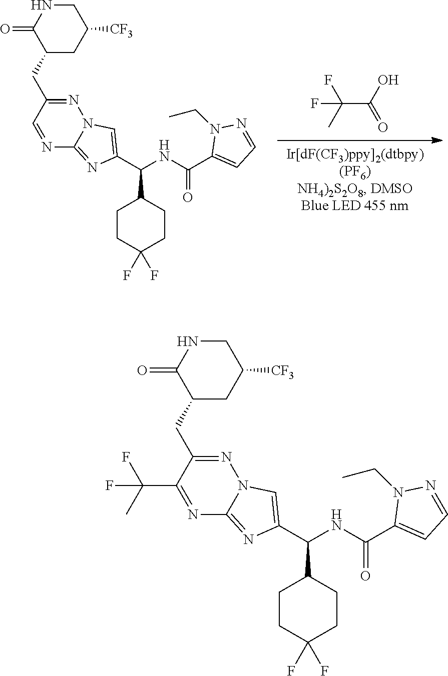

To a solution of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq) and 2,2-difluoropropanoic acid (33.8 mg, 307 mol, 2.50 eq) in DMSO (3.00 mL) was added Ir[dF(CF₃)ppy]₂(dtbpy)(PF₆) (1 mol %) (1.38 mg, 1.23 μmol, 0.0100 eq) followed by (NH₄)₂S₂O₈ (112 mg, 492 μmol, 107 μL, 4.00 eq). The reaction mixture was degassed and purged with N₂. The reaction mixture was stirred at 25° C. for 16 h irradiated with a 455 nm blue LED. The reaction mixture was diluted with Brine (10.0 mL) and extracted with ethyl acetate (3×10.0 mL). The combined organic layers were dried over anhydrous sodium sulfate, filtered and concentrated under reduced pressure to give a residue which was purified by Prep-TLC (SiO₂, petroleum ether:ethyl acetate=0:1) and then further purified by Prep-HPLC (column: Phenomenex luna C18 150 mm×25 mm, 10 um; mobile phase: [water (FA)—ACN]; gradient: 48%-68% B over 10 min) to afford N—((S)-(4,4-difluorocyclohexyl)(3-(1,1-difluoroethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (4.00 mg, 5.31 μmol, 4.31% yield, 83.9% purity) as a white solid.

### Example 177. Preparation of N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-4-methyl-1,2,5-oxadiazole-3-carboxamide (Compound 274 and 275)

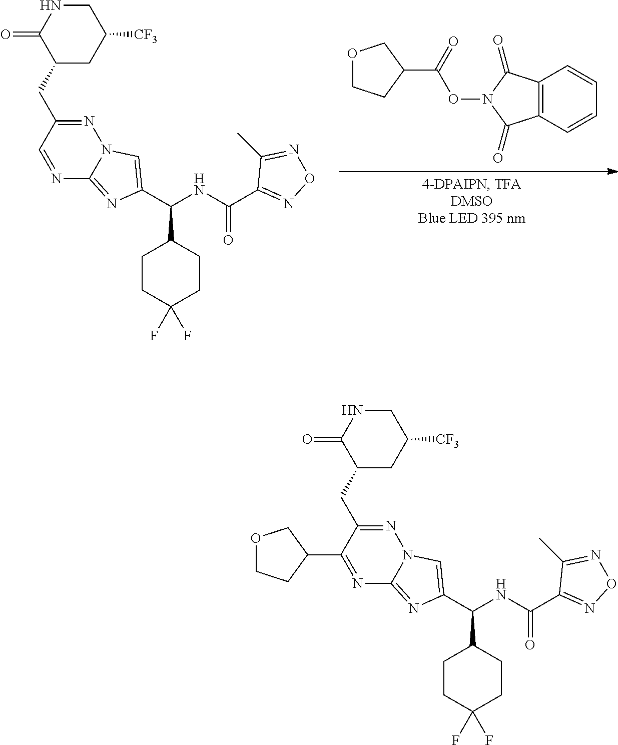 [SMILES: Cc1nonc1C(=O)N[C@H](c1cn2nc(C[C@H]3C[C@@H](C(F)(F)F)CNC3=O)cnc2n1)C1CCC(F)(F)CC1 ; O=C(ON1C(=O)c2ccccc2C1=O)C1CCOC1 ; "4-DPAIPN, TFADMSOBlue LED 395 nm" ; Cc1nonc1C(=O)N[C@H](c1cn2nc(C[C@H]3C[C@@H](C(F)(F)F)CNC3=O)c(C3CCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-4-methyl-1,2,5-oxadiazole-3-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (120 mg, 215 μmol, 1.00 eq), 1,3-dioxoisoindolin-2-yl tetrahydrofuran-3-carboxylate (112 mg, 431 μmol, 2.00 eq) and was purified by Prep-HPLC (FA condition; column: Phenomenex luna C18 150 mm×25 mm, 10 um; mobile phase: [water (FA)—ACN]; gradient: 46%-66% B over 10 min) to afford N-((1S)-(4,4-difluorocyclohexyl)(2-(((3R,5R)-2-oxo-5-(trifluoromethyl)piperidin-3-yl)methyl)-3-(tetrahydrofuran-3-yl)imidazo[1,2-b][1,2,4]triazin-6-yl)methyl)-4-methyl-1,2,5-oxadiazole-3-carboxamide (60.0 mg, 94.9 μmol, 44.0% yield, 99.2% purity) as a yellow solid.

Compound 274 was isolated as the first eluting, single stereoisomer by Prep-SFC (condition: column: Daicel Chiralcel OD-H (250 mm×30 mm, 5 um); mobile phase: [CO₂-i-PrOH]; B %: 30%, isocratic elution mode); (11.9 mg, 23.2 μmol, 24.2% yield, 97.0% purity) and was obtained as a yellow solid.

Compound 275 was isolated as the second eluting, single stereoisomer by Prep-SFC (condition: column: Daicel Chiralcel OD-H (250 mm×30 mm, 5 um); mobile phase: [CO₂-i-PrOH]; B %: 30%, isocratic elution mode); (15.68 mg, 23.2 μmol, 24.2% yield, 97.0% purity) and was obtained as a yellow solid.

### Example 178. Preparation of N—((S)-(4,4-difluorocyclohexyl)(3-(2-methoxyethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 276)

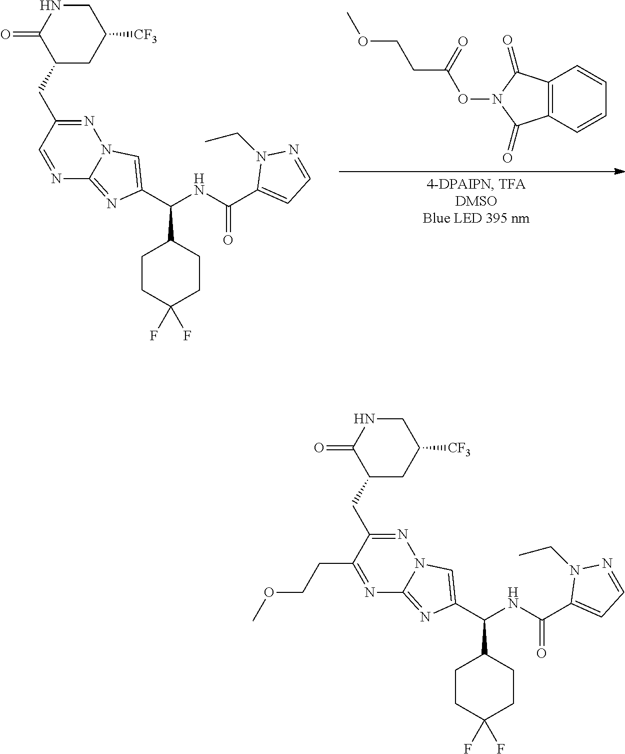 [SMILES: CCn1nccc1C(=O)N[C@H](c1cn2nc(C[C@H]3C[C@@H](C(F)(F)F)CNC3=O)cnc2n1)C1CCC(F)(F)CC1 ; COCCC(=O)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CCOC)nc2n1)C1CCC(F)(F)CC1]

N—((S)-(4,4-difluorocyclohexyl)(3-(2-methoxyethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq), 1,3-dioxoisoindolin-2-yl 3-methoxypropanoate (43.8 mg, 175 μmol, 2.00 eq) and was purified by Prep-HPLC (basic condition: column: Waters Xbridge 150 mm×25 mm, 5 um; mobile phase: [water (ammonia hydroxide v/v)—ACN]; gradient: 32%-62% B over 10 min) to afford N—((S)-(4,4-difluorocyclohexyl)(3-(2-methoxyethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (3.84 mg, 6.82 μmol, 85.5% purity) as a white solid.

### Example 179. Preparation of N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 67)

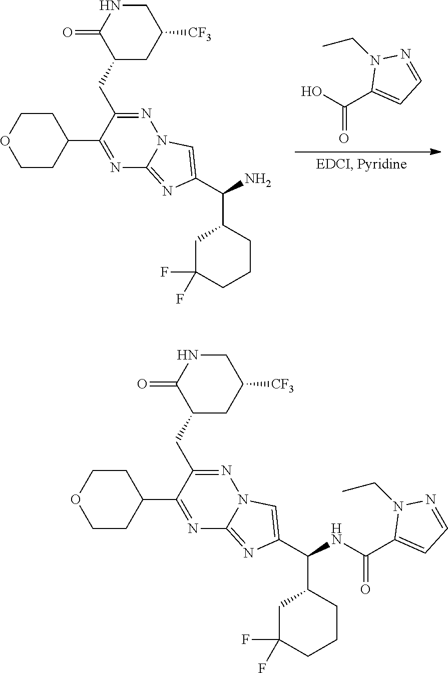

N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino((R)-3,3-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (800 mg, 1.51 mmol, 1.00 eq), 1-ethyl-1H-pyrazole-5-carboxylic acid (317 mg, 2.26 mmol, 1.50 eq) and was purified by column chromatography (SiO₂, Petroleum ether:Ethyl acetate=1:1 to 0:1) to afford N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (850 mg, 1.23 mmol, 81.79% yield, 94.7% purity) as a yellow solid.

### Example 180. Preparation of N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (Compound 277)

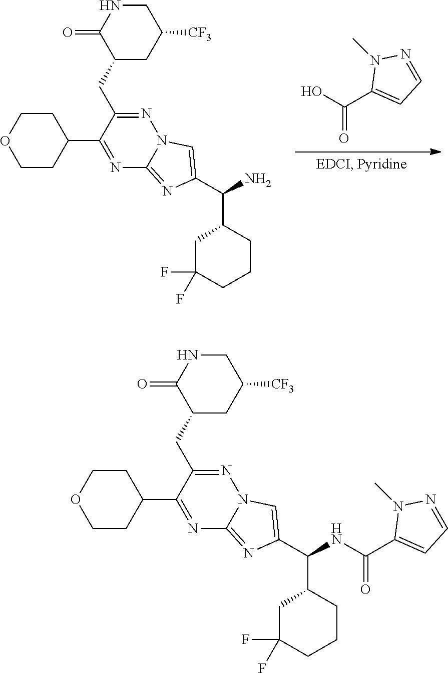

N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide was synthesized following the General Procedure 6 with (3R,5R)-3-((6-((S)-amino((R)-3,3-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 1-methyl-1H-pyrazole-5-carboxylic acid (17.8 mg, 141 μmol, 1.50 eq) and was purified by Prep-TLC (SiO₂, Dichloromethane:Methanol=10:1) to afford N—((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (30.05 mg, 45.61 μmol, 48.40% yield, 97.1% purity) as a yellow solid.

### Example 181. Preparation of N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (Compound 278)

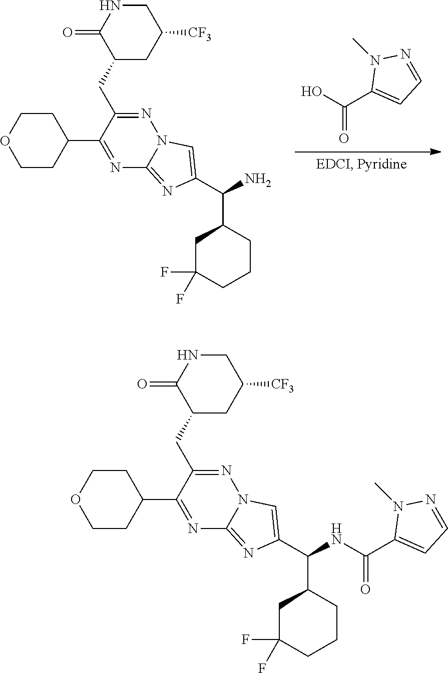

N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide was synthesized following the General Procedure 6 with (3R,5R)-3-((6-((S)-amino((S)-3,3-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (105 mg, 197 μmol, 1.00 eq), 1-methyl-1H-pyrazole-5-carboxylic acid (49.9 mg, 395 μmol, 2.00 eq) and was purified by Prep-HPLC (FA condition; column: Phenomenex luna C18 150 mm×25 mm, 10 mm; mobile phase: [water (FA) ACN]; gradient: 37%-67% B over 10 min) to afford N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (38.2 mg, 59.1 μmol, 29.8% yield, 98.8% purity) as a white solid.

### Example 182. Preparation of N—((S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 280)

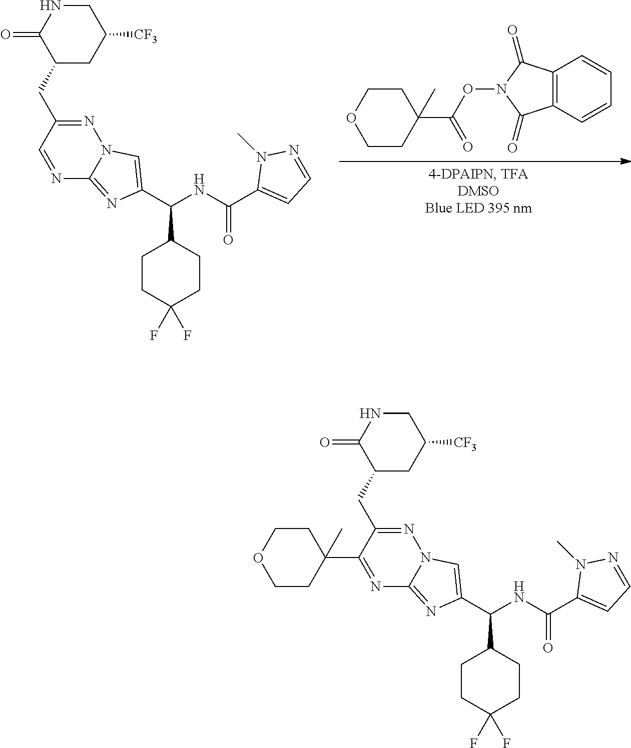

N—((S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (70.0 mg, 126 μmol, 1.00 eq), 1,3-dioxoisoindolin-2-yl 4-methyltetrahydro-2H-pyran-4-carboxylate (73.0 mg, 252 μmol, 2.00 eq) and was purified by Prep-HPLC (FA condition: column: Phenomenex luna C18 150 mm×25 mm, 10 um; mobile phase: [water (FA)—ACN]; gradient: 44%-60% B over 8 min) to afford N—((S)-(4,4-difluorocyclohexyl)(3-(4-methyltetrahydro-2H-pyran-4-yl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (6.21 mg, 9.24 μmol, 7.32% yield, 97.1% purity) as a yellow solid.

### Example 183. Preparation of N-((1S)-(4,4-difluorocyclohexyl)(2-(((3R,5R)-2-oxo-5-(trifluoromethyl)piperidin-3-yl)methyl)-3-pyrrolidin-3-yl)imidazo[1,2-b][1,2,4]triazin-6-yl)methyl)-1-ethyl-1H-pyrazole-5-carboxamide (Compound 284)

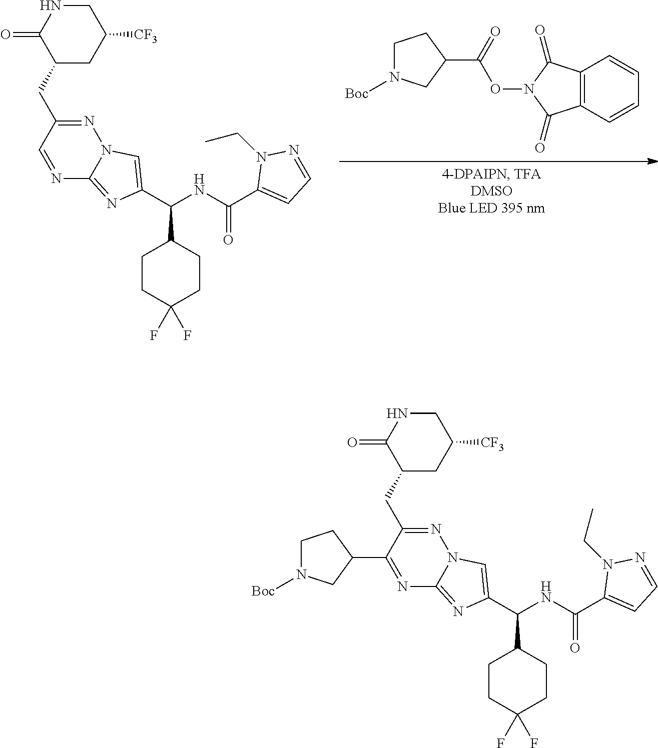 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCC(C(=O)ON2C(=O)c3ccccc3C2=O)C1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCN(C(=O)OC(C)(C)C)C3)nc2n1)C1CCC(F)(F)CC1]

Tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)pyrrolidine-1-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq), 1-(tert-butyl) 3-(1,3-dioxoisoindolin-2-yl) pyrrolidine-1,3-dicarboxylate (152 mg, 422 μmol, 2.00 eq) and was purified by Prep-HPLC (column: Phenomenex luna C18 150 mm×25 mm, 10 um; mobile phase: [water (FA)—ACN]; gradient: 46%-66% B over 10 min) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)pyrrolidine-1-carboxylate (8.00 mg, 10.4 μmol, 4.97% yield, 96.7% purity) as a white solid.

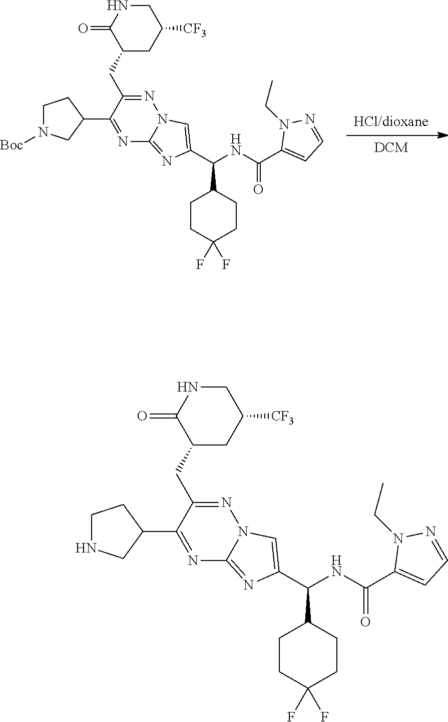

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)pyrrolidine-1-carboxylate (8.00 mg, 10.8 μmol, 1.00 eq) in dichloromethane (1.00 mL) was added HCl/dioxane (2.00 M, 5.42 μL, 1.00 eq). The reaction mixture was stirred at 25° C. for 1 h. The reaction mixture was filtered and concentrated under reduced pressure to afford N-((1S)-(4,4-difluorocyclohexyl)(2-(((3R,5R)-2-oxo-5-(trifluoromethyl)piperidin-3-yl)methyl)-3-(pyrrolidin-3-yl)imidazo[1,2-b][1,2,4]triazin-6-yl)methyl)-1-ethyl-1H-pyrazole-5-carboxamide (7.06 mg, 10.3 μmol, 94.9% yield, 93.0% purity) as a white solid.

### Example 184. Preparation of N-((1S)-(4,4-difluorocyclohexyl)(3-(1-methylpyrrolidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 439 and 285)

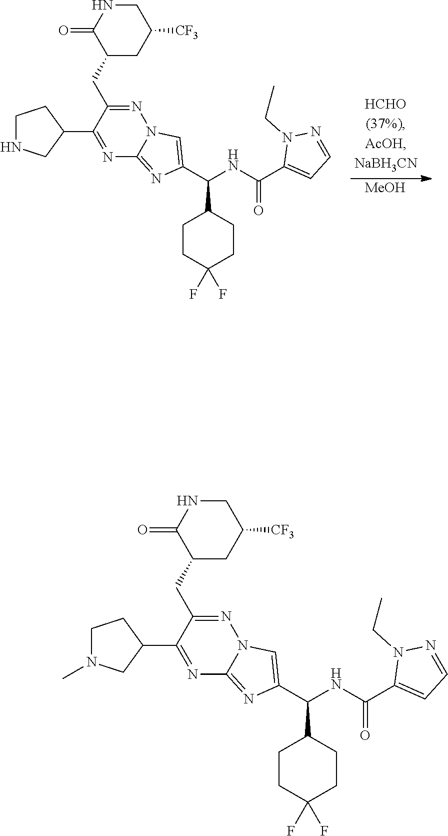 [SMILES: "MeOH" ; CCn1nccc1C(=O)N[C@H](c1cn2nc(C[C@H]3C[C@@H](C(F)(F)F)CNC3=O)c(C3CCNC3)nc2n1)C1CCC(F)(F)CC1 ; CCn1nccc1C(=O)N[C@H](c1cn2nc(C[C@H]3C[C@@H](C(F)(F)F)CNC3=O)c(C3CCN(C)C3)nc2n1)C1CCC(F)(F)CC1]

To a solution of N-((1S)-(4,4-difluorocyclohexyl)(2-(((3R,5R)-2-oxo-5-(trifluoromethyl)piperidin-3-yl)methyl)-3-(pyrrolidin-3-yl)imidazo[1,2-b][1,2,4]triazin-6-yl)methyl)-1-ethyl-1H-pyrazole-5-carboxamide (110 mg, 172 μmol, 1.00 eq, HCl salt) in methanol (1.50 mL) was added NaBH₃CN (37.9 mg, 603 μmol, 3.50 eq), AcOH (62.1 mg, 1.04 mmol, 59.2 μL, 6.00 eq) and formaldehyde (1.60 g, 19.7 mmol, 1.47 mL, 37% purity, 114 eq). The reaction mixture was stirred at 25° C. for 1 h. The reaction was concentrated under reduced pressure and the residue was purified by Prep-HPLC (column: Phenomenex luna C18 150 mm×25 mm, 10 um; mobile phase: [water (FA)—ACN]; gradient: 18%-38% B over 10 min) to afford N-((1S)-(4,4-difluorocyclohexyl)(3-(1-methylpyrrolidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 107 μmol, 62.2% yield) as a white solid.

Compound 439 was isolated as the first eluting, single stereoisomer by chiral SFC purification (column: Daicel Chiralcel OD-H (250 mm×30 mm, 5 um); mobile phase: [CO₂-i-PrOH (0.1% NH₃H₂O)]; B %: 25%, isocratic elution mode); (25.09 mg, 37.2 μmol, 34.6% yield, 96.7% purity) and was obtained as a white solid.

Compound 285 was isolated as the second eluting, single stereoisomer by chiral SFC purification (column: Daicel Chiralcel OD-H (250 mm×30 mm, 5 um); mobile phase: [CO₂-i-PrOH (0.1% NH₃H₂O)]; B %: 25%, isocratic elution mode); (32.86 mg, 49.8 μmol, 46.3% yield, 98.8% purity) and was obtained as a white solid.

### Example 185. Preparation of N-((1S)-(4,4-difluorocyclohexyl)(3-(1-methylpyrrolidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 370)

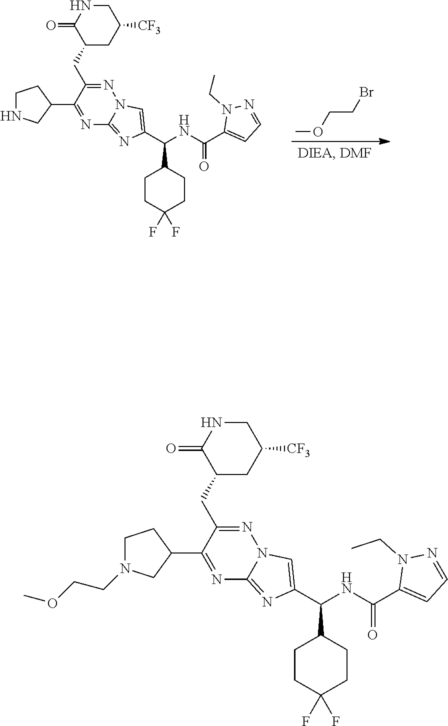

To a solution of N-((1S)-(4,4-difluorocyclohexyl)(2-(((3R,5R)-2-oxo-5-(trifluoromethyl)piperidin-3-yl)methyl)-3-(pyrrolidin-3-yl)imidazo[1,2-b][1,2,4]triazin-6-yl)methyl)-1-ethyl-1H-pyrazole-5-carboxamide (60.0 mg, 94.1 μmol, 1.00 eq, HCl salt) and 1-bromo-2-methoxyethane (19.6 mg, 141 μmol, 13.2 μL, 1.50 eq) in DMF (1.00 mL) was added DIEA (36.4 mg, 282 μmol, 49.1 μL, 3.00 eq). The reaction mixture was stirred at 25° C. for 2 h. The reaction was concentrated under reduced pressure and the residue was purified by Prep-HPLC (column: Phenomenex luna C18 150 mm×25 mm, 10 um; mobile phase: [water (FA)—ACN]; gradient: 20%-40% B over 10 min) and Prep-HPLC (column: Waters Xbridge 150 mm×25 mm, 5 um; mobile phase: [water (ammonia hydroxide v/v)—ACN]; gradient: 37%-67% B over 9 min) to afford N-((1S)-(4,4-difluorocyclohexyl)(3-(1-(2-methoxyethyl)pyrrolidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (22.97 mg, 32.1 μmol, 34.1% yield, 97.3% purity) as a white solid.

### Example 186. Preparation of N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 289)

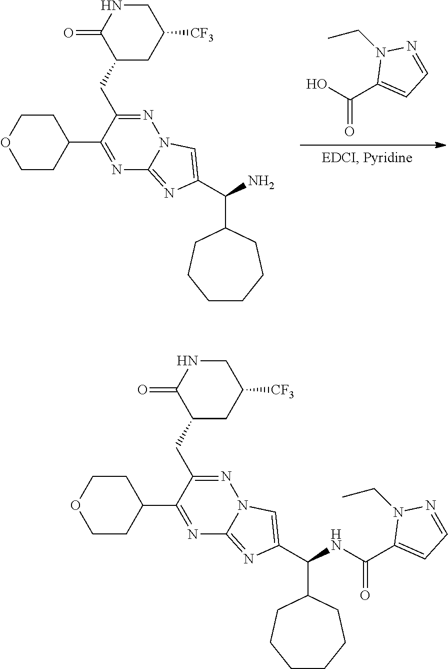

N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(cycloheptyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 78.6 μmol, 1.00 eq), 1-ethyl-1H-pyrazole-5-carboxylic acid (13.2 mg, 94.3 μmol, 1.20 eq) and was purified by Prep-HPLC (column: Phenomenex luna C18 150 mm×25 mm, 10 mm; mobile phase: [water (FA) ACN]; gradient: 50%-70% B over 10 min) to afford N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (22.37 mg, 35.2 μmol, 44.8% yield, 99.4% purity) as a white solid.

### Example 187. Preparation of N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (Compound 290)

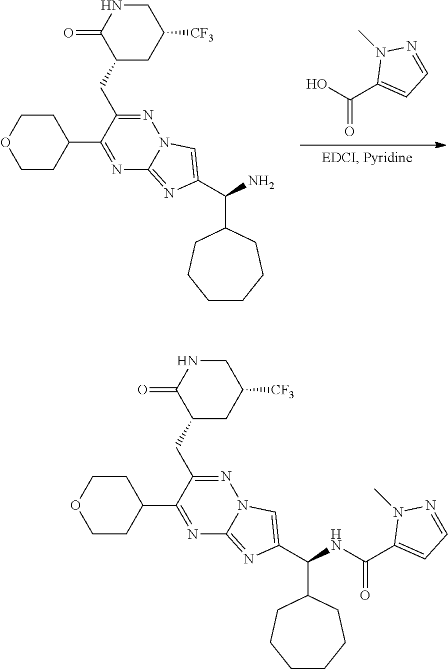

N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide was synthesized following the General Procedure 6 with (3R,5R)-3-((6-((S)-amino(cycloheptyl)methyl)-3-(tetrahydro-2H-pyran-4-yl)imidazo[1,2-b][1,2,4]triazin-2-yl)methyl)-5-(trifluoromethyl)piperidin-2-one (40.0 mg, 78.6 μmol, 1.00 eq), 1-methyl-1H-pyrazole-5-carboxylic acid (11.9 mg, 94.3 μmol, 1.20 eq) and was purified by Prep-HPLC (column: Phenomenex luna C18 150 mm×25 mm, 10 mm; mobile phase: [water (FA) ACN]; gradient: 46%-66% B over 10 min) to afford N—((S)-cycloheptyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-methyl-1H-pyrazole-5-carboxamide (15.49 mg, 24.3 μmol, 30.9% yield, 96.8% purity) as a white solid.

### Example 188. Preparation of N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 291)

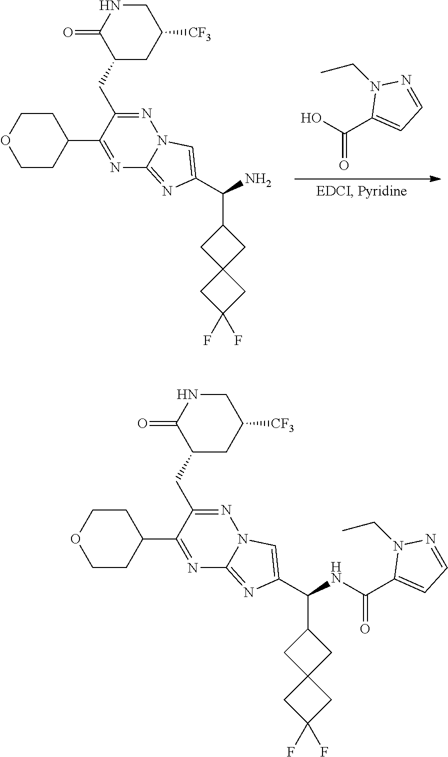

N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(6,6-difluorospiro[3.3]heptan-2-yl)methyl)-3-(tetrahydro-2H-pyran-4-yl)imidazo[1,2-b][1,2,4]triazin-2-yl)methyl)-5-(trifluoromethyl)piperidin-2-one (45.0 mg, 82.9 μmol, 1.00 eq), 1-ethyl-1H-pyrazole-5-carboxylic acid (15.1 mg, 107 μmol, 1.30 eq) and was purified by Prep-TLC (SiO₂, dichloromethane:methanol=10:1) and further purified by Prep-SFC (column: Regis (S,S)Whelk—O1 (250 mm×25 mm, 10 um); mobile phase: [CO₂-i-PrOH]; B %: 50%, isocratic elution mode) to afford N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (38.7 mg, 57.7 μmol, 69.6% yield, 99.2% purity) as a yellow solid.

### Example 189. Preparation of N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 314)

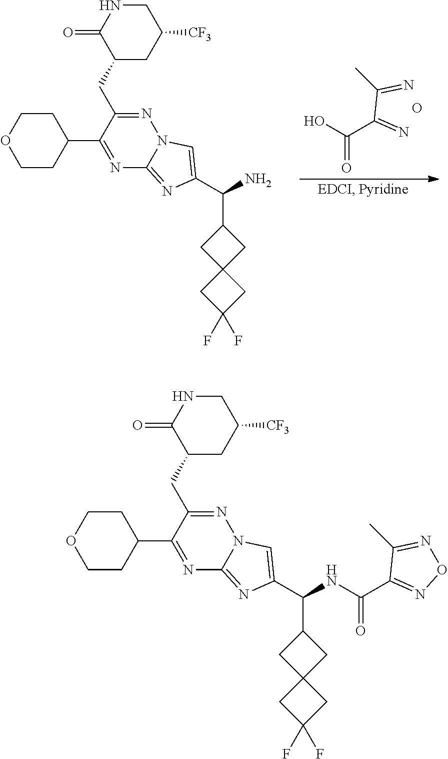

N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide was synthesized following the General Procedure 6 with (3R,5R)-3-((6-((S)-amino(6,6-difluorospiro[3.3]heptan-2-yl)methyl)-3-(tetrahydro-2H-pyran-4-yl)imidazo[1,2-b][1,2,4]triazin-2-yl)methyl)-5-(trifluoromethyl)piperidin-2-one (8.00 mg, 14.7 μmol, 1.00 eq), 4-methyl-1,2,5-oxadiazole-3-carboxylic acid (2.46 mg, 19.1 μmol, 1.30 eq) and was purified by Prep-HPLC (column: Phenomenex luna C18 150 mm×25 mm, 10 um; mobile phase: [water (FA)—ACN]; gradient: 48%-68% B over 10 min) to afford N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (3.38 mg, 5.14 μmol, 34.88% yield, 99.3% purity) as a white solid.

### Example 190. Preparation of N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (Compound 315)

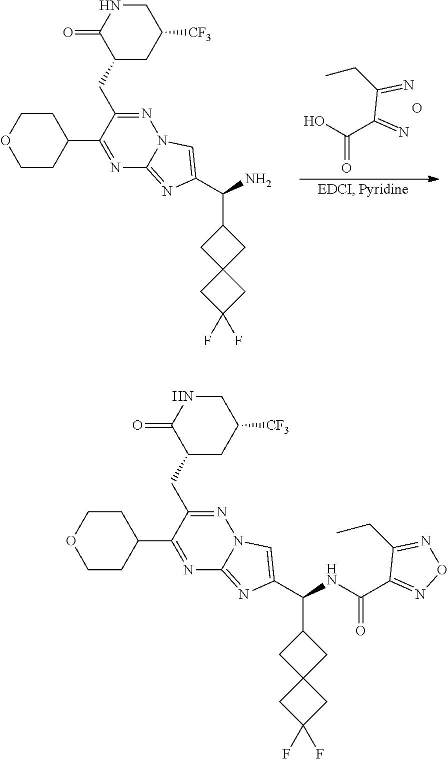

N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide was synthesized following the General Procedure 6 with (3R,5R)-3-((6-((S)-amino(6,6-difluorospiro[3.3]heptan-2-yl)methyl)-3-(tetrahydro-2H-pyran-4-yl)imidazo[1,2-b][1,2,4]triazin-2-yl)methyl)-5-(trifluoromethyl)piperidin-2-one (8.00 mg, 14.7 μmol, 1.00 eq), 4-ethyl-1,2,5-oxadiazole-3-carboxylic acid (2.72 mg, 19.1 μmol, 1.30 eq) and was purified by Prep-HPLC (column: Phenomenex luna C18 150 mm×25 mm, 10 um; mobile phase: [water (FA)—ACN]; gradient: 52%-72% B over 10 min) to afford N—((S)-(6,6-difluorospiro[3.3]heptan-2-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (2.82 mg, 4.16 μmol, 28.2% yield, 98.3% purity) as a white solid.

### Example 191. Preparation of N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 292)

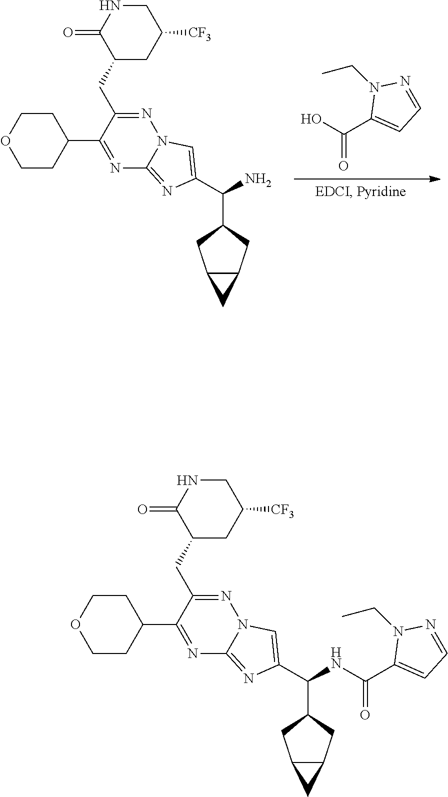

N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino((1R,3r,5S)-bicyclo[3.1.0]hexan-3-yl)methyl)-3-(tetrahydro-2H-pyran-4-yl)imidazo[1,2-b][1,2,4]triazin-2-yl)methyl)-5-(trifluoromethyl)piperidin-2-one (30.0 mg, 60.9 μmol, 1.00 eq), 1-ethyl-1H-pyrazole-5-carboxylic acid (12.8 mg, 91.4 μmol, 1.50 eq) and was purified by Prep-TLC (SiO₂, dichloromethane:methanol=10:1) and further purified by Prep-SFC (column: Regis (S,S)Whelk—O1 (250 mm×25 mm, 10 um); mobile phase: [CO₂-i-PrOH], B %: 50%, isocratic elution mode) to afford N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (1.75 mg, 2.79 μmol, 4.59% yield, 98.1% purity) as a yellow solid.

### Example 192. Preparation of N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 316)

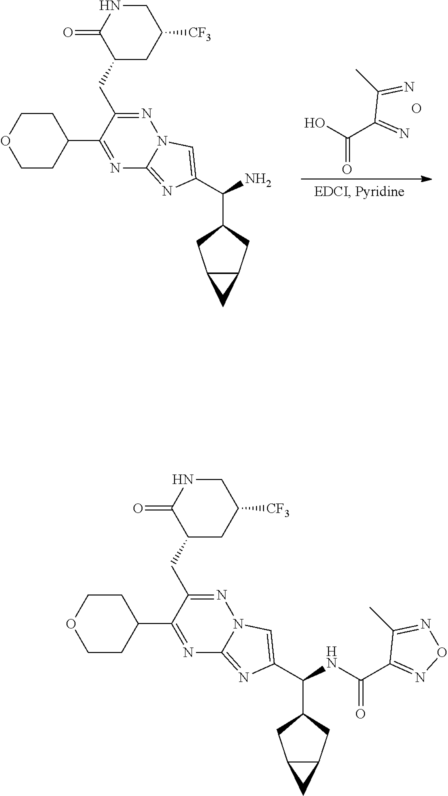

N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide was synthesized following the General Procedure 6 with (3R,5R)-3-((6-((S)-amino((1R,3r,5S)-bicyclo[3.1.0]hexan-3-yl)methyl)-3-(tetrahydro-2H-pyran-4-yl)imidazo[1,2-b][1,2,4]triazin-2-yl)methyl)-5-(trifluoromethyl)piperidin-2-one (30.0 mg, 60.9 μmol, 1.00 eq), 4-methyl-1,2,5-oxadiazole-3-carboxylic acid (11.7 mg, 91.4 μmol, 1.50 eq) and was purified by Prep-HPLC (column: Phenomenex luna C18 150 mm×25 mm, 10 um; mobile phase: [water (FA)—ACN]; gradient: 47%-67% B over 10 min) to afford N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (8.08 mg, 12.8 μmol, 21.0% yield, 95.5% purity) as a white solid.

### Example 193. Preparation of N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 317)

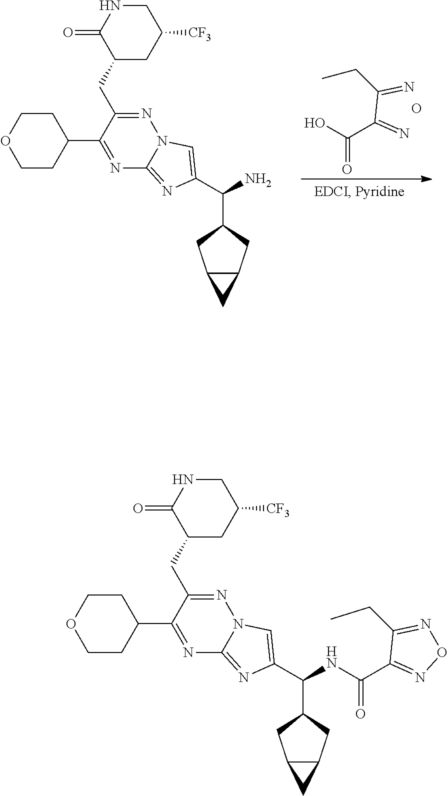

N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide was synthesized following the General Procedure 6 with (3R,5R)-3-((6-((S)-amino((1R,3r,5S)-bicyclo[3.1.0]hexan-3-yl)methyl)-3-(tetrahydro-2H-pyran-4-yl)imidazo[1,2-b][1,2,4]triazin-2-yl)methyl)-5-(trifluoromethyl)piperidin-2-one (30.0 mg, 60.9 μmol, 1.00 eq), 4-ethyl-1,2,5-oxadiazole-3-carboxylic acid (12.9 mg, 91.4 μmol, 1.50 eq) and was purified by Prep-HPLC (column: Phenomenex luna C18 150 mm×25 mm, 10 um; mobile phase: [water (FA)—ACN]; gradient: 50%-70% B over 10 min) to afford N—((S)-((1R,3r,5S)-bicyclo[3.1.0]hexan-3-yl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (7.30 mg, 11.8 μmol, 19.4% yield, 100% purity) as a white solid.

### Example 194. Preparation of N-(bicyclo[5.1.0]octan-4-yl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (Compound 293 and 294)

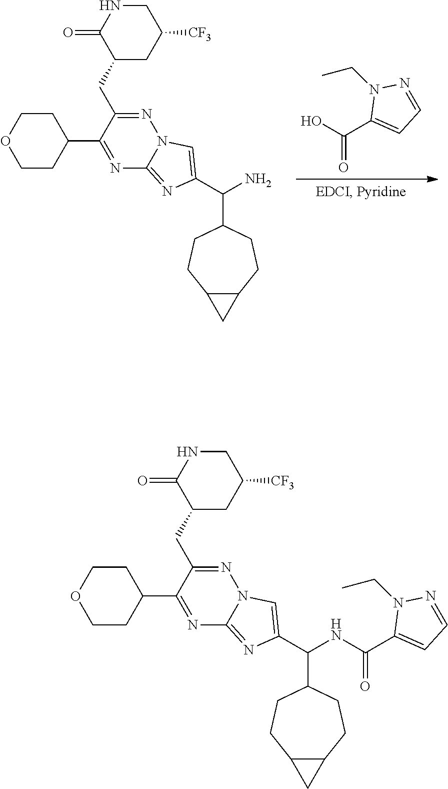

N-(bicyclo[5.1.0]octan-4-yl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide was synthesized following the General Procedure 6 with (3R,5R)-3-((6-(amino(bicyclo[5.1.0]octan-4-yl)methyl)-3-(tetrahydro-2H-pyran-4-yl)imidazo[1,2-b][1,2,4]triazin-2-yl)methyl)-5-(trifluoromethyl)piperidin-2-one (100 mg, 192 μmol, 1.00 eq), 1-ethyl-1H-pyrazole-5-carboxylic acid (32.3 mg, 230 μmol, 1.20 eq) and was purified by Prep-HPLC (column: Phenomenex Luna C18 150 mm×25 mm, 10 um; mobile phase: [water (FA)—ACN]; gradient: 40%-70% B over 10 min) to afford N-(bicyclo[5.1.0]octan-4-yl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-5-carboxamide (123 mg) as a white solid.

Compound 293 was isolated as the first eluting, single stereoisomer by chiral SFC purification (column: Regis (S,S) Whelko1 (250 mm×25 mm, 10 um); mobile phase: [CO₂-i-PrOH/ACN]; B %: 40%, isocratic elution mode); (10.88 mg, 16.13 μmol, 10.37% yield, 95.3% purity) and was obtained as a yellow solid.

Compound 294 was isolated as the second eluting, single stereoisomer by chiral SFC purification (column: Regis (S,S) Whelko1 (250 mm×25 mm, 10 um); mobile phase: [CO₂-i-PrOH/ACN]; B %: 40%, isocratic elution mode); (25.07 mg, 37.76 μmol, 24.27% yield, 96.8% purity) and was obtained as a yellow solid.

### Example 195. Preparation of N—((S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 295)

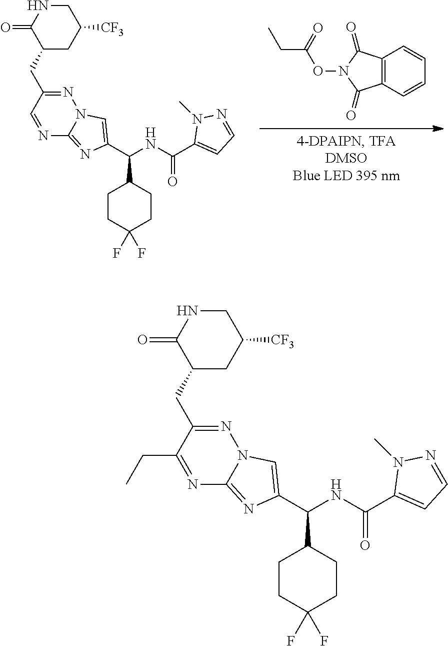 [SMILES: Cn1nccc1C(=O)N[C@H](c1cn2nc(C[C@H]3C[C@@H](C(F)(F)F)CNC3=O)cnc2n1)C1CCC(F)(F)CC1 ; CCC(=O)ON1C(=O)c2ccccc2C1=O ; "4-DPAIPN, TFADMSOBlue LED 395 nm" ; CCc1nc2nc([C@@H](NC(=O)c3ccnn3C)C3CCC(F)(F)CC3)cn2nc1C[C@H]1C[C@@H](C(F)(F)F)CNC1=O]

N—((S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (60.0 mg, 108 μmol, 1.00 eq), 1,3-dioxoisoindolin-2-yl propionate (59.2 mg, 270 μmol, 2.50 eq) and was purified by Prep-HPLC (column: Phenomenex luna C18 150 mm×25 mm, 10 um; mobile phase: [water (FA)—ACN]; gradient: 41%-61% B over 10 min) and further purified by Prep-SFC (column: Daicel Chiralpak AS (250 mm×30 mm, 10 um); mobile phase: [CO₂-i-PrOH]; B %:45%, isocratic elution mode) to afford N—((S)-(4,4-difluorocyclohexyl)(3-ethyl-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (24.68 mg, 41.1 μmol, 38.0% yield, 97.2% purity) as a white solid.

### Example 196. Preparation of N—((S)-(4,4-difluorocyclohexyl)(2-(((3R,5R)-2-oxo-5-(trifluoromethyl)piperidin-3-yl)methyl)-3-(piperidin-4-yl)imidazo[1,2-b][1,2,4]triazin-6-yl)methyl)-1-ethyl-1H-pyrazole-5-carboxamide (Compound 299)

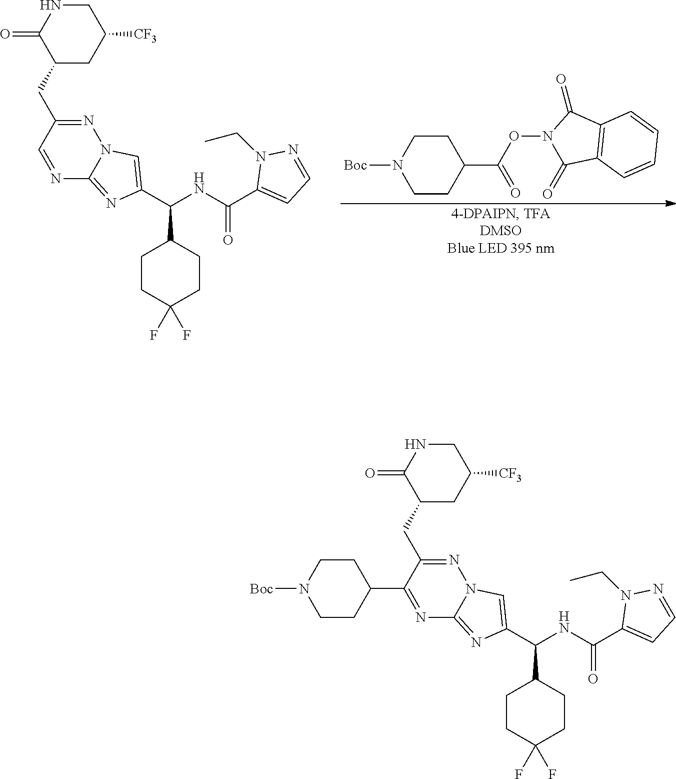

Tert-butyl 4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (300 mg, 527 μmol, 1.00 eq), 1-(tert-butyl) 4-(1,3-dioxoisoindolin-2-yl) piperidine-1,4-dicarboxylate (395 mg, 1.06 mmol, 2.00 eq) and was purified by Prep-TLC (SiO₂, Dichloromethane:Methanol=10:1) to afford tert-butyl 4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (300 mg, 399 μmol, 75.6% yield) as a yellow solid.

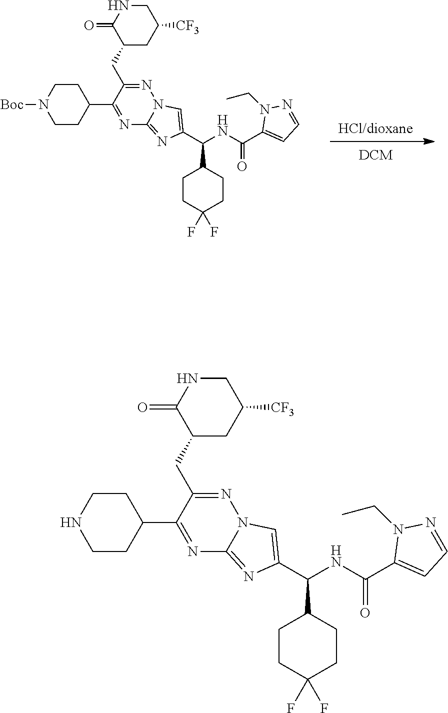

To a solution of tert-butyl 4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (100 mg, 133.02 μmol, 1.00 eq) in dichloromethane (1.00 mL) was added HCl/dioxane (2 M, 0.5 mL, 7.52 eq). The reaction mixture was stirred at 25° C. for 0.5 h. The reaction mixture was concentrated under reduced pressure to give a residue which was purified by Prep-HPLC (basic condition: column: Waters Bridge C18 150 mm×50 mm, 10 um; mobile phase: [water (NH₄HCO₃)-ACN]; gradient: 22%-52% B over 10 min) to afford N—((S)-(4,4-difluorocyclohexyl)(2-(((3R,5R)-2-oxo-5-(trifluoromethyl)piperidin-3-yl)methyl)-3-(piperidin-4-yl)imidazo[1,2-b][1,2,4]triazin-6-yl)methyl)-1-ethyl-1H-pyrazole-5-carboxamide (31.24 mg, 45.4 μmol, 34.1% yield, 94.8% purity) as a yellow solid.

### Example 197. Preparation of N—((S)-(4,4-difluorocyclohexyl)(3-(1-methylpiperidin-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 300)

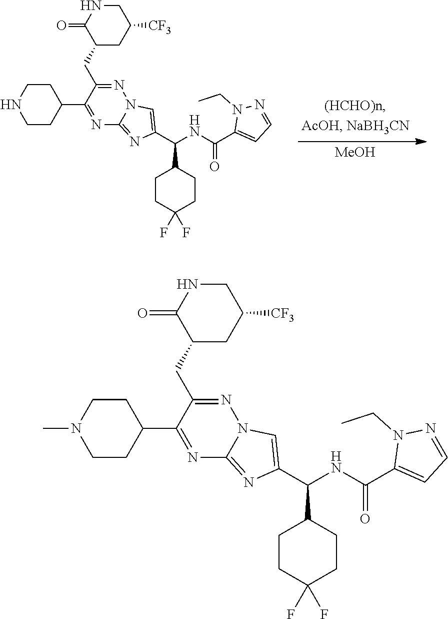 [SMILES: "MeOH" ; CCn1nccc1C(=O)N[C@H](c1cn2nc(C[C@H]3C[C@@H](C(F)(F)F)CNC3=O)c(C3CCNCC3)nc2n1)C1CCC(F)(F)CC1 ; CCn1nccc1C(=O)N[C@H](c1cn2nc(C[C@H]3C[C@@H](C(F)(F)F)CNC3=O)c(C3CCN(C)CC3)nc2n1)C1CCC(F)(F)CC1]

To a solution of N—((S)-(4,4-difluorocyclohexyl)(2-(((3R,5R)-2-oxo-5-(trifluoromethyl)piperidin-3-yl)methyl)-3-(piperidin-4-yl)imidazo[1,2-b][1,2,4]triazin-6-yl)methyl)-1-ethyl-1H-pyrazole-5-carboxamide (100 mg, 145 μmol, 1.00 eq, HCl salt) in MeOH (2.00 mL) as added (HCHO)n (1.53 g, 18.8 mmol, 1.41 mL, 37% purity, 130 eq), NaBH₃CN (31.9 mg, 508 μmol, 3.50 eq) and acetic acid (52.3 mg, 871 μmol, 49.9 μL, 6.00 eq). The reaction mixture was stirred at 25° C. for 0.5 h. The reaction mixture was diluted with water (30.0 mL) and extracted with EtOAc (3×30.0 mL), dried with anhydrous sodium sulfate, filtrated and concentrated under reduced pressure to give a residue, which was purified by prep-HPLC (neutral condition; column: Waters Xbridge C18 150 mm×50 mm, 10 um; mobile phase: [water(NH₄HCO₃)—ACN]; gradient:22%-52% B over 10 min) to afford N—((S)-(4,4-difluorocyclohexyl)(3-(1-methylpiperidin-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (27.54 mg, 40.2 μmol, 27.6% yield, 97.1% purity) as a yellow solid.

### Example 198. Preparation of N—((S)-(3-(azetidin-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 301)

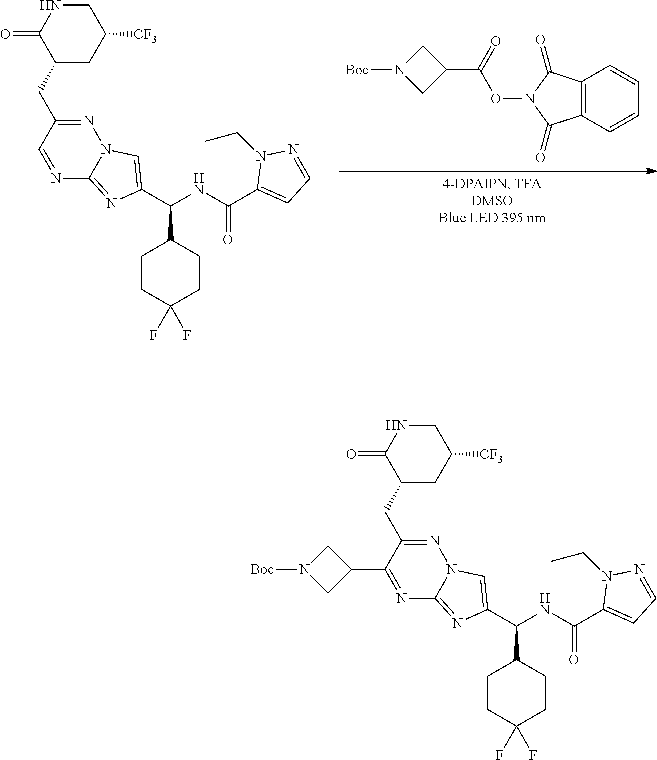 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CC(C(=O)ON2C(=O)c3ccccc3C2=O)C1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CN(C(=O)OC(C)(C)C)C3)nc2n1)C1CCC(F)(F)CC1]

Tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)azetidine-1-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq), 1-(tert-butyl) 3-(1,3-dioxoisoindolin-2-yl) azetidine-1,3-dicarboxylate (121 mg, 351 μmol, 2.00 eq) and was purified by Prep-TLC (SiO₂, Petroleum ether:Ethyl acetate=1:3) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)azetidine-1-carboxylate (50.0 mg, 69.0 μmol, 39.2% yield) as a yellow solid.

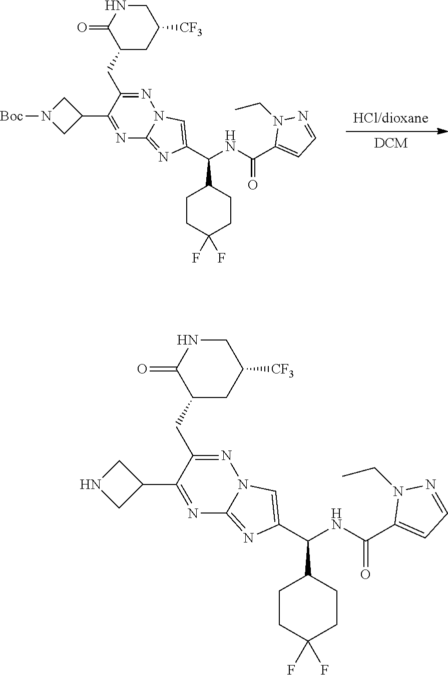

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)azetidine-1-carboxylate (40.0 mg, 55.2 μmol, 1.00 eq) in dichloromethane (1.00 mL) was added HCl/dioxane (2 M, 276 μL, 10.0 eq). The reaction mixture was stirred at 25° C. for 1 h. The reaction mixture was concentrated under reduced pressure to give a residue which was purified by Prep-HPLC (basic condition: column: Waters Bridge C18 150 mm×25 mm, 5 um; mobile phase: [water (ammonia hydroxide v/v)—ACN]; gradient: 17%-47% B over 9 min) to afford N—((S)-(3-(azetidin-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (5.56 mg, 8.19 μmol, 14.8% yield, 91.9% purity) as a yellow solid.

### Example 199. Preparation of N—((S)-(4,4-difluorocyclohexyl)(3-(1-methylazetidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 302)

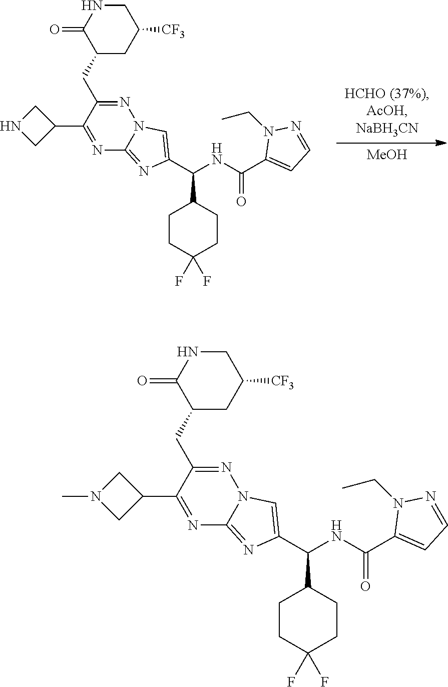 [SMILES: "MeOH" ; CCn1nccc1C(=O)N[C@H](c1cn2nc(C[C@H]3C[C@@H](C(F)(F)F)CNC3=O)c(C3CNC3)nc2n1)C1CCC(F)(F)CC1 ; "HCHO (37%),AcOH,NaBH3CN" ; CCn1nccc1C(=O)N[C@H](c1cn2nc(C[C@H]3C[C@@H](C(F)(F)F)CNC3=O)c(C3CN(C)C3)nc2n1)C1CCC(F)(F)CC1]

To a solution of N—((S)-(3-(azetidin-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (60.0 mg, 90.9 μmol, 1.00 eq, HCl salt) in methanol (2.00 mL) was added NaBH₃CN, AcOH (32.7 mg, 545 μmol, 31.2 μL, 6.00 eq) and formaldehyde. The reaction mixture was concentrated under reduced pressure to give a residue which was purified by Prep-HPLC (neutral condition, column: Waters Xbridge 150 mm×25 mm, 5 um; mobile phase: [water (NH₄HCO₃)—ACN]; gradient: 21%-41% B over 10 min) to afford N—((S)-(4,4-difluorocyclohexyl)(3-(1-methylazetidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (16.43 mg, 24.3 μmol, 26.8% yield, 94.6% purity) as a yellow solid.

### Example 200. Preparation of N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 313)

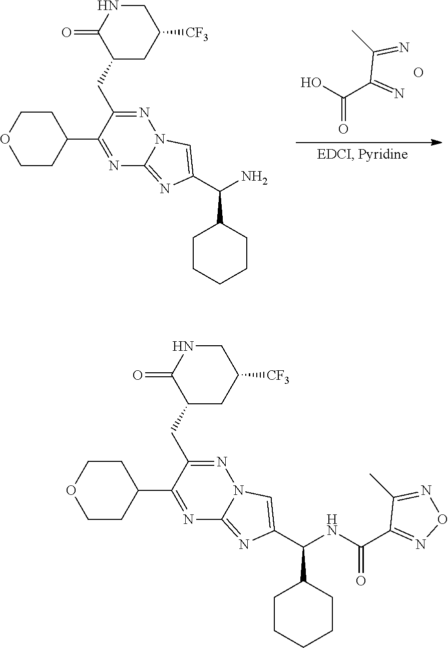

N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide was synthesized following the General Procedure 6 with (3R,5R)-3-((6-((S)-amino(cyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 101 μmol, 1.00 eq), 4-methyl-1,2,5-oxadiazole-3-carboxylic acid (38.8 mg, 303 μmol, 3.00 eq) and was purified by Prep-SFC (column: Daicel Chiralpak IG (250 mm×30 mm, 10 um); mobile phase: [CO₂-EtOH (0.1% NH₃H₂O)]; B %: 50%, isocratic elution mode) to afford N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (24.5 mg, 39.2 μmol, 38.7% yield, 96.5% purity) as a white solid.

### Example 201. Preparation of N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (Compound 221)

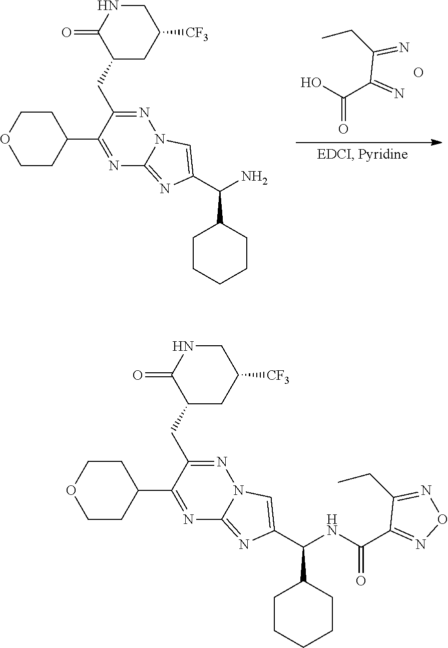

N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide was synthesized following the General Procedure 6 with (3R,5R)-3-((6-((S)-amino(cyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 101 μmol, 1.00 eq), 4-ethyl-1,2,5-oxadiazole-3-carboxylic acid (43.1 mg, 303 μmol, 3.00 eq) and was purified by Prep-SFC (column: Daicel Chiralpak AD (250 mm×30 mm, 10 um); mobile phase: [CO₂-i-PrOH]; B %: 35%, isocratic elution mode) to afford N—((S)-cyclohexyl(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (27.1 mg, 43.3 μmol, 42.8% yield, 98.6% purity) as a white solid.

### Example 202. Preparation of N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (Compound 318)

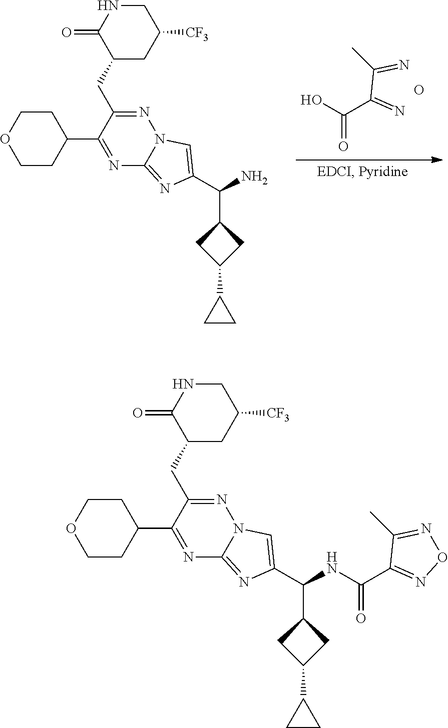

N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide was synthesized following the General Procedure 6 with (3R,5R)-3-((6-((S)-amino((1r,3S)-3-cyclopropylcyclobutyl)methyl)-3-(tetrahydro-2H-pyran-4-yl)imidazo[1,2-b][1,2,4]triazin-2-yl)methyl)-5-(trifluoromethyl)piperidin-2-one (25.0 mg, 49.3 μmol, 1.00 eq), 4-methyl-1,2,5-oxadiazole-3-carboxylic acid (7.59 mg, 59.2 μmol, 1.20 eq) and was purified by Prep-TLC (SiO₂, Dichloromethane:Methanol=100:1 to 10:1) to afford N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-methyl-1,2,5-oxadiazole-3-carboxamide (3.89 mg, 5.57 μmol, 11.2% yield, 88.3% purity) as a white solid.

### Example 203. Preparation of N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (Compound 319)

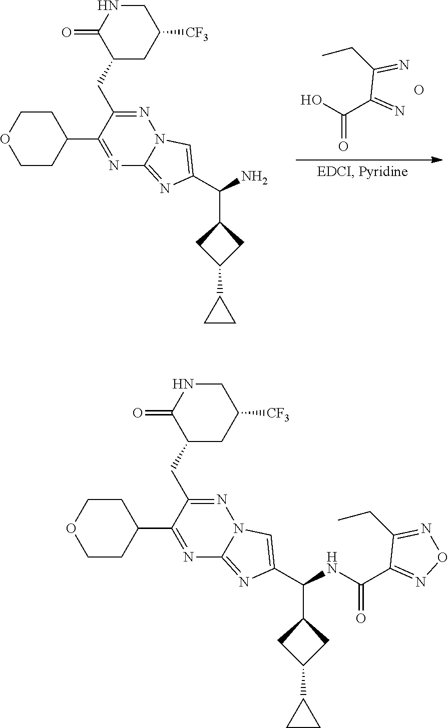

N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide was synthesized following the General Procedure 6 with (3R,5R)-3-((6-((S)-amino((1r,3S)-3-cyclopropylcyclobutyl)methyl)-3-(tetrahydro-2H-pyran-4-yl)imidazo[1,2-b][1,2,4]triazin-2-yl)methyl)-5-(trifluoromethyl)piperidin-2-one (25.0 mg, 49.3 μmol, 1.00 eq), 4-ethyl-1,2,5-oxadiazole-3-carboxylic acid (8.42 mg, 59.2 μmol, 1.20 eq) and was purified by Prep-TLC (SiO₂, Dichloromethane:Methanol=100:1 to 10:1) to afford N—((S)-((1r,3S)-3-cyclopropylcyclobutyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-ethyl-1,2,5-oxadiazole-3-carboxamide (13.4 mg, 20.0 μmol, 40.6% yield, 94.4% purity) as a white solid.

### Example 204. Preparation of N—((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-methyl-1H-pyrazole-5-carboxamide (Compound 320)

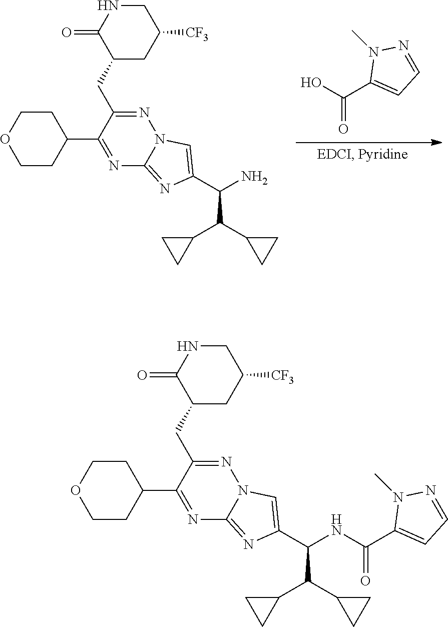

N—((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-methyl-1H-pyrazole-5-carboxamide was synthesized following the General Procedure 6 with (3R,5R)-3-((6-((S)-1-amino-2,2-dicyclopropylethyl)-3-(tetrahydro-2H-pyran-4-yl)imidazo[1,2-b][1,2,4]triazin-2-yl)methyl)-5-(trifluoromethyl)piperidin-2-one (130 mg, 256 μmol, 1.00 eq), 1-methyl-1H-pyrazole-5-carboxylic acid (38.8 mg, 307 μmol, 1.20 eq) and was purified by Prep-HPLC (column: Phenomenex luna C18 150 mm×25 mm, 10 mm; mobile phase: [water (FA) ACN]; gradient: 46%-66% B over 10 min) to afford N—((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-methyl-1H-pyrazole-5-carboxamide (93.8% purity) as a white solid.

### Example 205. Preparation of N-((1S)-2,2-dicyclopropyl-1-(2-(((3R,5R)-2-oxo-5-(trifluoromethyl)piperidin-3-yl)methyl)-3-(tetrahydrofuran-3-yl)imidazo[1,2-b][1,2,4]triazin-6-yl)ethyl)-1-ethyl-1H-pyrazole-5-carboxamide (Compound 321 and 322)

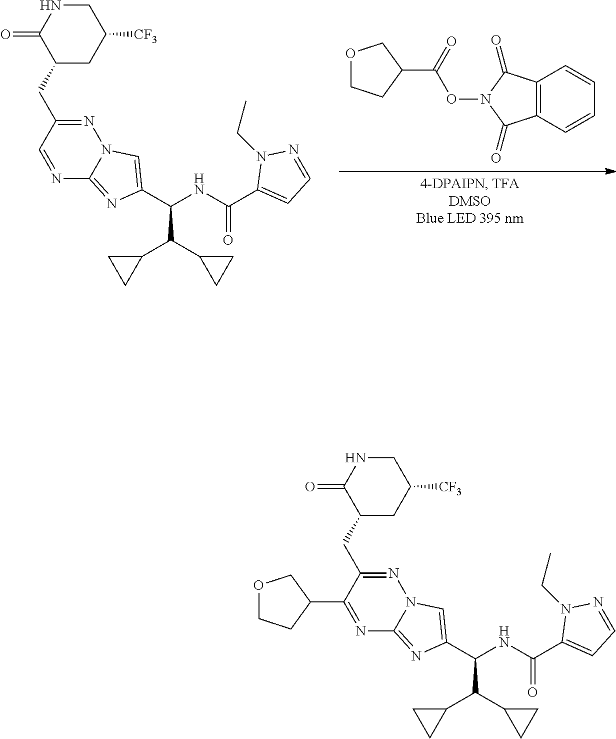 [SMILES: CCn1nccc1C(=O)N[C@H](c1cn2nc(C[C@H]3C[C@@H](C(F)(F)F)CNC3=O)cnc2n1)C(C1CC1)C1CC1 ; O=C(ON1C(=O)c2ccccc2C1=O)C1CCOC1 ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C3CCOC3)nc2n1)C(C1CC1)C1CC1]

N-((1S)-2,2-dicyclopropyl-1-(2-(((3R,5R)-2-oxo-5-(trifluoromethyl)piperidin-3-yl)methyl)-3-(tetrahydrofuran-3-yl)imidazo[1,2-b][1,2,4]triazin-6-yl)ethyl)-1-ethyl-1H-pyrazole-5-carboxamide was synthesized following the General Procedure 7 with N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (135 mg, 247 μmol, 1.00 eq), 1,3-dioxoisoindolin-2-yl tetrahydrofuran-3-carboxylate (129 mg, 495 μmol, 2.00 eq) and was purified by Prep-HPLC (column: Waters X bridge 150 mm×25 mm, 5 um; mobile phase: [water (ammonia hydroxide v/v)—ACN]; gradient: 40%-47% B over 10 min) to afford N-((1S)-2,2-dicyclopropyl-1-(2-(((3R,5R)-2-oxo-5-(trifluoromethyl)piperidin-3-yl)methyl)-3-(tetrahydrofuran-3-yl)imidazo[1,2-b][1,2,4]triazin-6-yl)ethyl)-1-ethyl-1H-pyrazole-5-carboxamide (10.0 mg, 15.0 μmol, 6.06% yield, 92.3% purity) as a white solid.

Compound 321 was isolated as the first eluting, single stereoisomer by Prep-SFC (column: Daicel Chiralcel OD (250 mm×30 mm, 10 um); mobile phase: [CO₂-i-PrOH/ACN]; B %: 35%, isocratic elution mode); (2.39 mg, 3.74 μmol, 22.9% yield, 96.2% purity) and was obtained as a white solid.

Compound 322 was isolated as the second eluting, single stereoisomer by Prep-SFC (column: Daicel Chiralcel OD (250 mm×30 mm, 10 um); mobile phase: [CO₂-i-PrOH/ACN]; B %: 35%, isocratic elution mode); (3.42 mg, 5.16 μmol, 31.7% yield, 92.8% purity) and was obtained as a white solid.

### Example 206. Preparation of N-((1S)-(4,4-difluorocyclohexyl)(2-(((3R,5R)-2-oxo-5-(trifluoromethyl)piperidin-3-yl)methyl)-3-(piperidin-2-yl)imidazo[1,2-b][1,2,4]triazin-6-yl)methyl)-1-ethyl-1H-pyrazole-5-carboxamide (Compound 324 and 325)

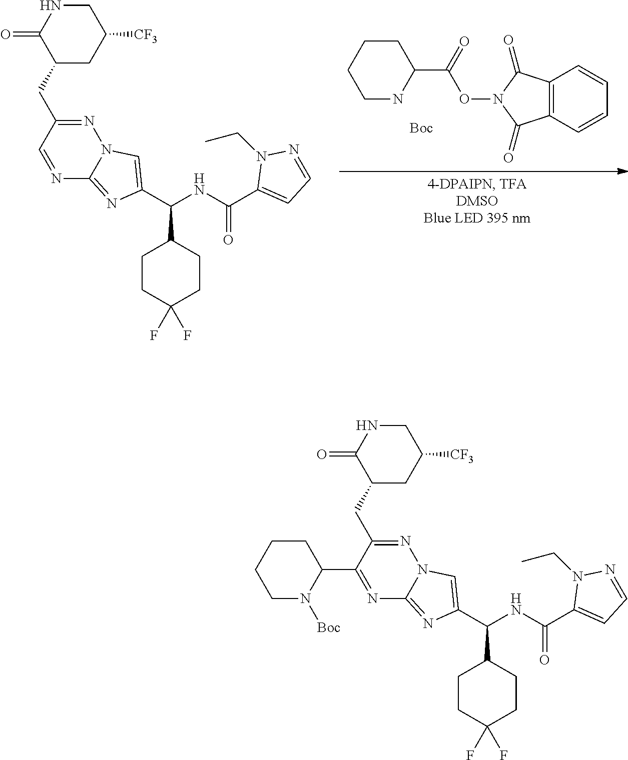

Tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (200 mg, 352 μmol, 1.00 eq), 1,3-dioxoisoindolin-2-yl tetrahydrofuran-3-carboxylate (263 mg, 703 μmol, 2.00 eq) and was purified by Prep-HPLC (column: Phenomenex luna C18 150 mm×25 mm, 10 um; mobile phase: [water (FA)—ACN]; gradient: 54%-74% B over 10 min) to afford tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (36.0 mg, 47.9 μmol, 13.6% yield, 100% purity) as a yellow solid.

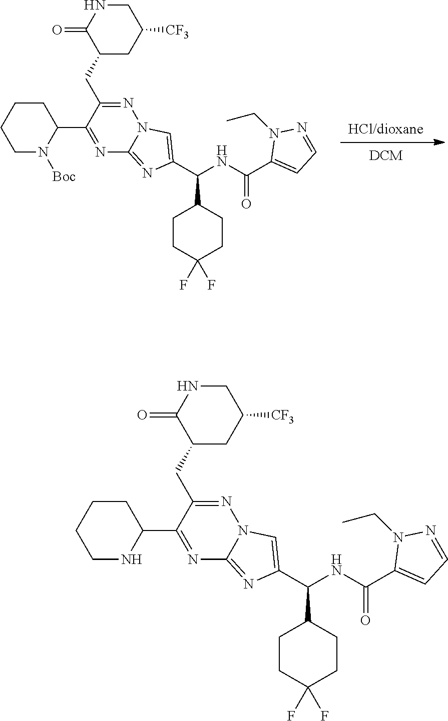

To a solution of tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (36.0 mg, 47.9 μmol, 1 eq) in dichloromethane (1.00 mL) was added HCl/dioxane (2.00 M, 2.00 mL, 83.5 eq). The reaction mixture was stirred at 0° C. for 1 h. The reaction mixture was concentrated under reduced pressure to afford N-((1S)-(4,4-difluorocyclohexyl)(2-(((3R,5R)-2-oxo-5-(trifluoromethyl)piperidin-3-yl)methyl)-3-(piperidin-2-yl)imidazo[1,2-b][1,2,4]triazin-6-yl)methyl)-1-ethyl-1H-pyrazole-5-carboxamide (30.0 mg, 43.6 μmol, 91.0% yield, HCl salt) as a yellow solid.

Compound 324 was isolated as the first eluting, single stereoisomer by Prep-SFC (column: Regis (S,S) Whelk—O1 (250 mm×25 mm, 10 um); mobile phase: [CO₂-i-PrOH (0.1% NH₃H₂O)]; B %: 50%, isocratic elution mode); (4.24 mg, 5.95 μmol, 13.7% yield, 91.5% purity) and was obtained as a white solid.

Compound 325 was isolated as the second eluting, single stereoisomer by Prep-SFC (column: Regis (S,S) Whelk—O1 (250 mm×25 mm, 10 um); mobile phase: [CO₂-i-PrOH (0.1% NH₃H₂O)]; B %: 50%, isocratic elution mode); (7.23 mg, 9.74 μmol, 22.3% yield, 87.8% purity) and was obtained as a white solid.

### Example 207. Preparation of N—((S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide (Compound 329 and 330)

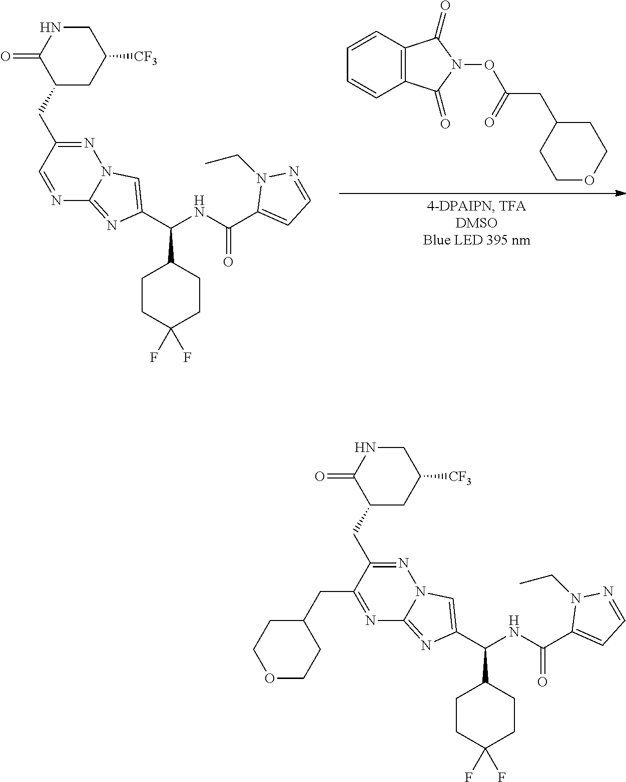 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCOCC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCOCC3)nc2n1)C1CCC(F)(F)CC1]

N—((S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydro-2H-pyran-4-yl)acetate (39.1 mg, 135 μmol, 1.10 eq) and was purified by Prep-HPLC (column: Phenomenex luna C18 150 mm×25 mm, 10 um; mobile phase: [water (FA)—ACN]; gradient: 42%-62% B over 10 min) to afford N—((S)-(4,4-difluorocyclohexyl)(2-(((3R,5R)-2-oxo-5-(trifluoromethyl)piperidin-3-yl)methyl)-3-((tetrahydro-2H-pyran-4-yl)methyl)imidazo[1,2-b][1,2,4]triazin-6-yl)methyl)-1-ethyl-1H-pyrazole-5-carboxamide (18.0 mg, 26.9 μmol, 21.8% yield, 99.7% purity) as a yellow solid.

Compound 329 was isolated as the first eluting, single stereoisomer by chiral SFC purification (column: Daicel Chiralpak IE (250 mm×30 mm, 10 um); mobile phase: [Heptane-EtOH (0.1% TFA)]; B %: 80%, isocratic elution mode); (5.00 mg, 7.50 μmol, 18.5% yield, 86.1% purity) and was obtained as a white solid.

Compound 330 was isolated as the second eluting, single stereoisomer by chiral SFC purification (column: Daicel Chiralpak IE (250 mm×30 mm, 10 um); mobile phase: [Heptane-EtOH (0.1% TFA)]; B %: 80%, isocratic elution mode); (5.00 mg, 7.50 μmol, 18.5% yield, 85.7% purity) and was obtained as a white solid.

### Example 208. Preparation of N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide (Compound 224 and 225)

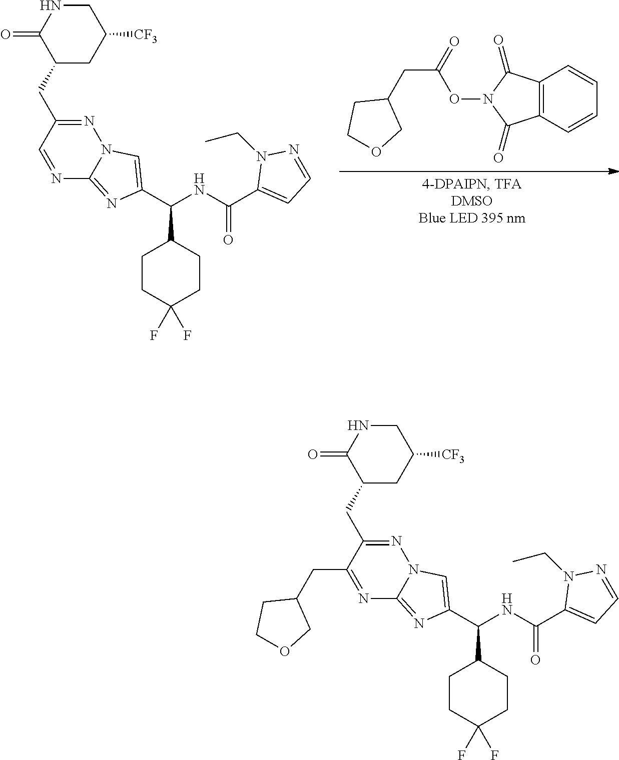 [SMILES: CCn1nccc1C(=O)N[C@H](c1cn2nc(C[C@H]3C[C@@H](C(F)(F)F)CNC3=O)cnc2n1)C1CCC(F)(F)CC1 ; O=C(CC1CCOC1)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(CC3CCOC3)nc2n1)C1CCC(F)(F)CC1]

N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (70.0 mg, 123 μmol, 1.00 eq), 1,3-dioxoisoindolin-2-yl 2-(tetrahydrofuran-3-yl)acetate (67.7 mg, 246 μmol, 2.00 eq) and was purified by Prep-HPLC (column: Phenomenex luna C18 150 mm×25 mm, 10 um; mobile phase: [water (FA)—ACN]; gradient: 40%-60% B over 10 min) to afford N-((1S)-(4,4-difluorocyclohexyl)(2-(((3R,5R)-2-oxo-5-(trifluoromethyl)piperidin-3-yl)methyl)-3-((tetrahydrofuran-3-yl)methyl)imidazo[1,2-b][1,2,4]triazin-6-yl)methyl)-1-ethyl-1H-pyrazole-5-carboxamide (5.00 mg, 7.34 μmol, 5.96% yield, 95.8% purity) as a white solid.

Compound 224 was isolated as the first eluting, single stereoisomer by chiral SFC purification (column: Daicel Chiralpak IG (250 mm×30 mm, 10 um); mobile phase: [CO₂-i-PrOH]; B %: 30%, isocratic elution mode); (1.88 mg, 2.61 μmol, 21.3% yield, 90.6% purity) and was obtained as a white solid.

Compound 225 was isolated as the second eluting, single stereoisomer by chiral SFC purification (column: Daicel Chiralpak IG (250 mm×30 mm, 10 um); mobile phase: [CO₂-i-PrOH]; B %: 30%, isocratic elution mode); (2.21 mg, 2.99 μmol, 24.4% yield, 88.4% purity) and was obtained as a white solid.

### Example 209. Preparation of N-((1S)-(4,4-difluorocyclohexyl)(3-(morpholin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 353)

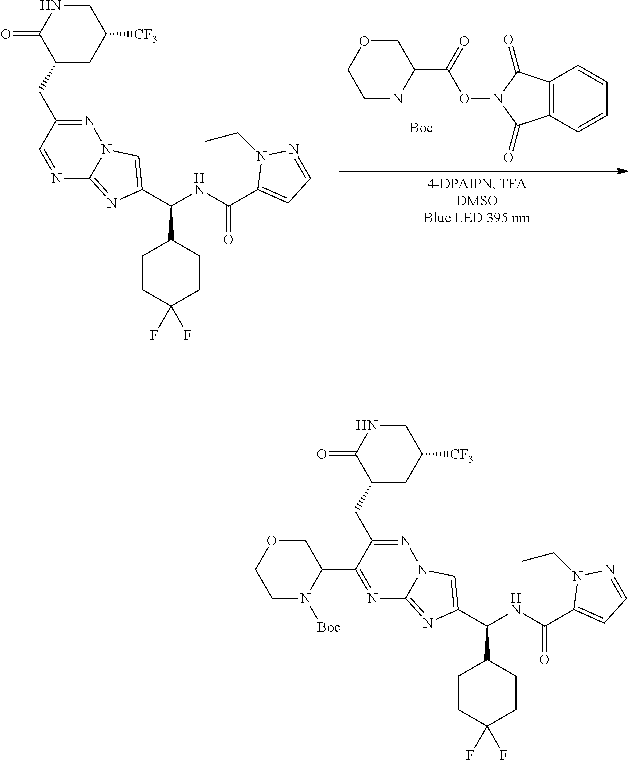

Tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (400 mg, 703 μmol, 1.00 eq), 4-(tert-butyl) 3-(1,3-dioxoisoindolin-2-yl) morpholine-3,4-dicarboxylate (250 mg, 664 μmol, 0.94 eq) and was purified by Prep-HPLC (column: Phenomenex luna C18 150 mm×25 mm, 10 um; mobile phase: [water (FA)—ACN]; gradient: 50%-70% B over 10 min) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate (200 mg, 265 μmol, 37.7% yield) as a white solid.

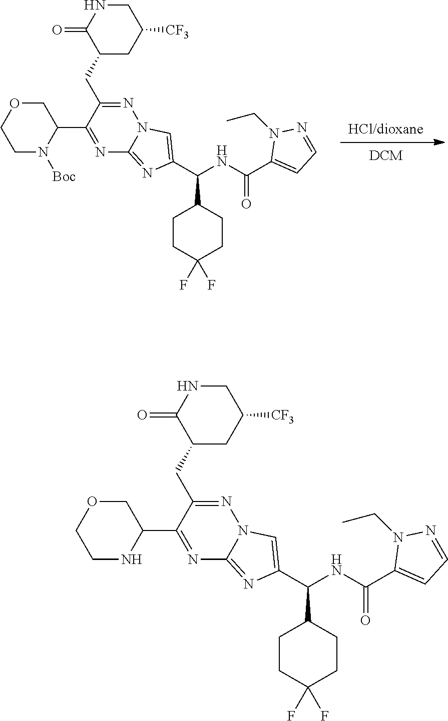

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate (190 mg, 252 μmol, 1.00 eq) in dichloromethane (1.00 mL) was added HCl/dioxane (12.0 M, 1.00 mL, 47.6 eq). The reaction mixture was stirred at 25° C. for 1 h. The reaction mixture was concentrated under reduced pressure to afford N-((1S)-(4,4-difluorocyclohexyl)(3-(morpholin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (130 mg, 198 μmol, 78.7% yield, 99.8% purity) as a white solid.

### Example 210. Preparation of N-((1S)-(4,4-difluorocyclohexyl)(3-(4-methylmorpholin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 404)

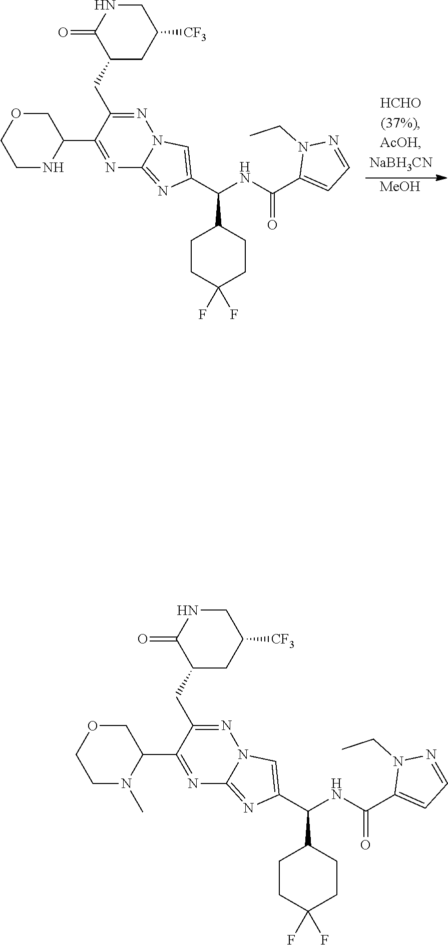 [SMILES: "MeOH" ; CCn1nccc1C(=O)N[C@H](c1cn2nc(C[C@H]3C[C@@H](C(F)(F)F)CNC3=O)c(C3COCCN3)nc2n1)C1CCC(F)(F)CC1 ; CCn1nccc1C(=O)N[C@H](c1cn2nc(C[C@H]3C[C@@H](C(F)(F)F)CNC3=O)c(C3COCCN3C)nc2n1)C1CCC(F)(F)CC1]

To a solution of N-((1S)-(4,4-difluorocyclohexyl)(3-(morpholin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 152 μmol, 1.00 eq) in methyl alcohol (2.00 mL) was added AcOH (55.1 mg, 917 μmol, 52.5 μL, 6.00 eq), NaBH₃CN (33.6 mg, 535 μmol, 3.50 eq) and formaldehyde (2.18 g, 26.8 mmol, 2.00 mL, 37.0% purity, 175 eq). The reaction mixture was stirred at 25° C. for 1 hr. The residue was diluted with water and extracted with ethyl acetate. The combined organic layers were dried over anhydrous sodium sulfate, filtered and concentrated under reduced pressure to give a residue which was purified by Prep-HPLC (column: Phenomenex luna C18 150 mm×25 mm, 10 um; mobile phase: [water (FA)—ACN]; gradient: 26%-36% B over 10 min) (column: Phenomenex luna C 18 150*25 mm*10 um; mobile phase: [water (FA)—ACN]; gradient: 19%-39% B over 10 min) to afford N-((1S)-(4,4-difluorocyclohexyl)(3-(4-methylmorpholin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide.

Compound 404 was isolated as the second eluting, single stereoisomer by chiral SFC purification (column: Daicel Chiralpak AD (250 mm×30 mm, 10 um); mobile phase: [CO₂-i-PrOH/ACN]; B %: 25%, isocratic elution mode); (30.63 mg, 43.58 μmol, 28.49%) and was obtained as a white solid.

### Example 211. Preparation of tert-butyl 8-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.5]nonane-2-carboxylate (Compound 240)

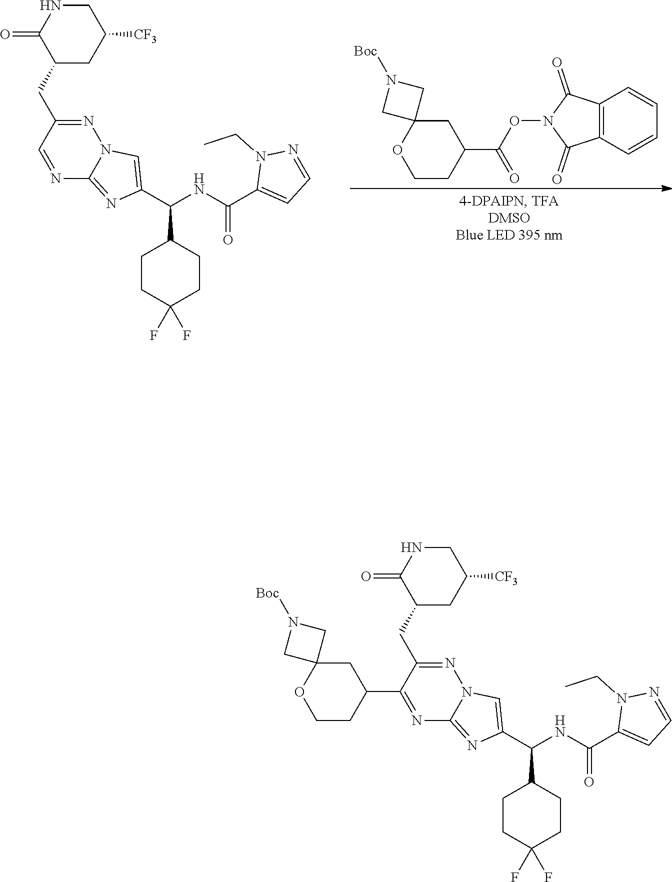 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CC2(CC(C(=O)ON3C(=O)c4ccccc4C3=O)CCO2)C1 ; CCn1nccc1C(=O)N[C@H](c1cn2nc(C[C@H]3C[C@@H](C(F)(F)F)CNC3=O)c(C3CCOC4(C3)CN(C(=O)OC(C)(C)C)C4)nc2n1)C1CCC(F)(F)CC1]

Tert-butyl 8-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.5]nonane-2-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (200 mg, 351 μmol, 1.00 eq), 2-(tert-butyl) 8-(1,3-dioxoisoindolin-2-yl) 5-oxa-2-azaspiro[3.5]nonane-2,8-dicarboxylate (292 mg, 703 μmol, 2.00 eq) and was purified by Prep-HPLC (FA condition; column: Phenomenex luna C18 150 mm×25 mm, 10 um; mobile phase: [water (FA)—ACN]; gradient: 44%-74% B over 10 min) to afford tert-butyl 8-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.5]nonane-2-carboxylate (120 mg, 151 μmol, 42.9% yield) as a yellow solid.

### Example 212. Preparation of N-((1S)-(4,4-difluorocyclohexyl)(3-(2-methyl-5-oxa-2-azaspiro[3.5]nonan-8-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 354)

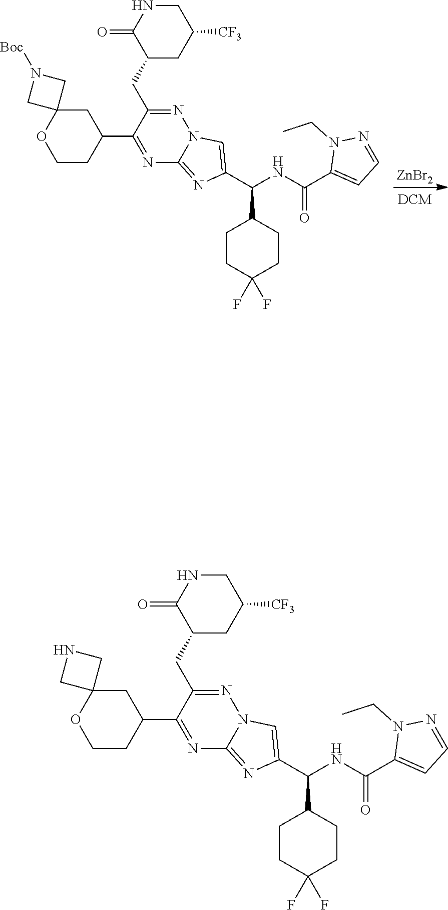

To a solution of tert-butyl 8-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.5]nonane-2-carboxylate (100 mg, 125 μmol, 1.00 eq) in dichloromethane (2.00 mL) was added ZnBr₂ (113 mg, 503 μmol, 25.2 μL, 4.00 eq). The reaction mixture was stirred at 25° C. for 12 h. The reaction mixture was concentrated under reduced pressure to afford N-((1S)-(4,4-difluorocyclohexyl)(2-(((3R,5R)-2-oxo-5-(trifluoromethyl)piperidin-3-yl)methyl)-3-(5-oxa-2-azaspiro[3.5]nonan-8-yl)imidazo[1,2-b][1,2,4]triazin-6-yl)methyl)-1-ethyl-1H-pyrazole-5-carboxamide (80.0 mg, 115 μmol, 91.5% yield) as a yellow solid.

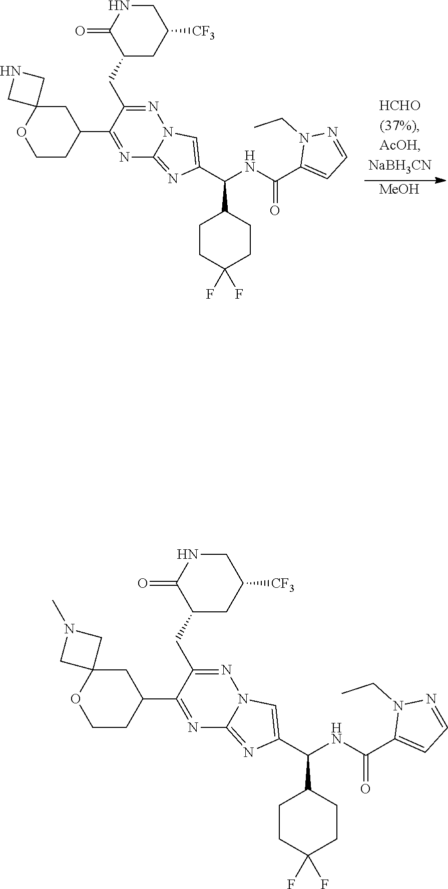 [SMILES: "MeOH" ; CCn1nccc1C(=O)N[C@H](c1cn2nc(C[C@H]3C[C@@H](C(F)(F)F)CNC3=O)c(C3CCOC4(CNC4)C3)nc2n1)C1CCC(F)(F)CC1 ; CCn1nccc1C(=O)N[C@H](c1cn2nc(C[C@H]3C[C@@H](C(F)(F)F)CNC3=O)c(C3CCOC4(C3)CN(C)C4)nc2n1)C1CCC(F)(F)CC1]

To a solution of N-((1S)-(4,4-difluorocyclohexyl)(2-(((3R,5R)-2-oxo-5-(trifluoromethyl)piperidin-3-yl)methyl)-3-(5-oxa-2-azaspiro[3.5]nonan-8-yl)imidazo[1,2-b][1,2,4]triazin-6-yl)methyl)-1-ethyl-1H-pyrazole-5-carboxamide (80.0 mg, 99.0 μmol, 1.00 eq) in MeOH (3.00 mL) was added HCHO (1.04 g, 12.8 mmol, 958 μL, 37.0% purity, 130 eq), NaBH₃CN (21.7 mg, 346 μmol, 3.50 eq), AcOH (35.6 mg, 594 μmol, 34.0 μL, 6.00 eq). The reaction mixture was stirred at 25° C. for 0.5 h. The reaction mixture was concentrated under reduced pressure to give a residue which was purified by Prep-HPLC (FA condition: column: Phenomenex luna C18 150 mm×25 mm, 10 um; mobile phase: [water (FA)—ACN]; gradient: 20%-40% B over 10 min) to afford N-((1S)-(4,4-difluorocyclohexyl)(3-(2-methyl-5-oxa-2-azaspiro[3.5]nonan-8-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (35.78 mg, 49.5 μmol, 50.0% yield, 98.0% purity) as an off-white solid.

### Example 213. Preparation of N-((1S)-(4,4-difluorocyclohexyl)(3-(2-methyl-5-oxa-2-azaspiro[3.4]octan-7-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 355)

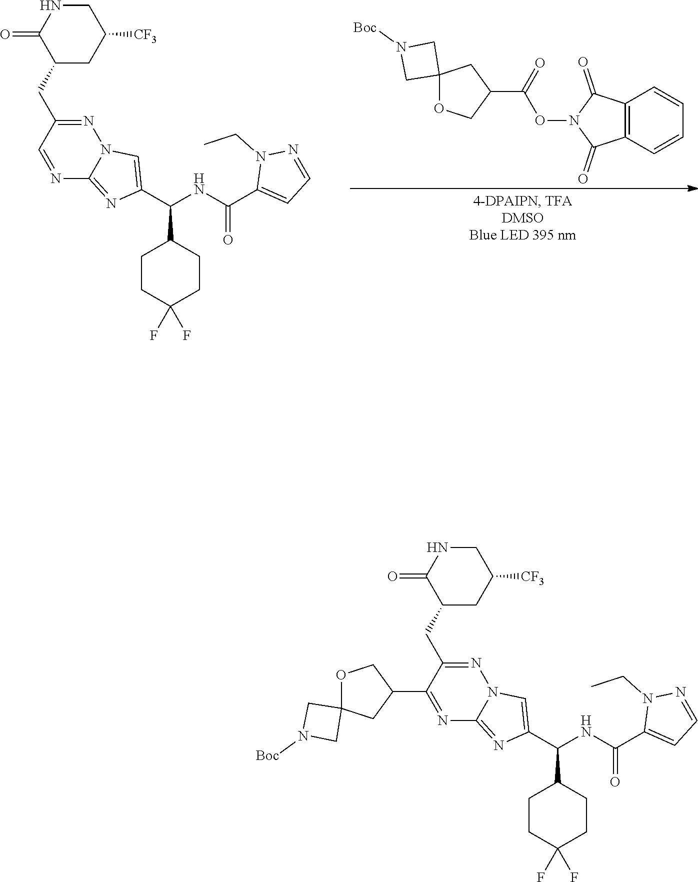 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CC2(CC(C(=O)ON3C(=O)c4ccccc4C3=O)CO2)C1 ; CCn1nccc1C(=O)N[C@H](c1cn2nc(C[C@H]3C[C@@H](C(F)(F)F)CNC3=O)c(C3COC4(C3)CN(C(=O)OC(C)(C)C)C4)nc2n1)C1CCC(F)(F)CC1]

Tert-butyl 7-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.4]octane-2-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide 200 mg, 351 μmol, 1.00 eq), 2-(tert-butyl) 7-(1,3-dioxoisoindolin-2-yl) 5-oxa-2-azaspiro[3.4]octane-2,7-dicarboxylate (283 mg, 703 μmol, 2.00 eq) and was purified by Prep-HPLC (neutral condition; column: Waters Xbridge C18 150 mm×50 mm, 10 um; mobile phase: [water (NH₄HCO₃)—ACN]; gradient: 38%-68% B over 10 min) to afford tert-butyl 7-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.4]octane-2-carboxylate (160 mg, 205 μmol, 58.3% yield) as a yellow solid.

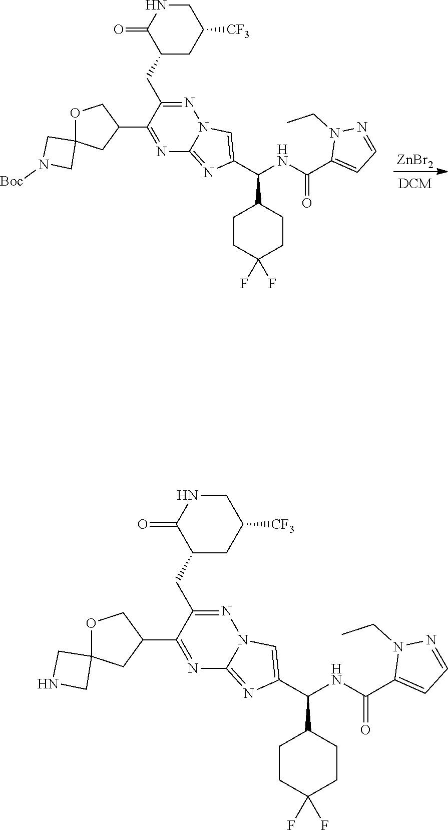

To a solution of tert-butyl 7-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-5-oxa-2-azaspiro[3.4]octane-2-carboxylate (50.0 mg, 64.1 μmol, 1.00 eq) in dichloromethane (1.00 mL) was added ZnBr₂ (57.7 mg, 256 μmol, 12.8 μL, 4.00 eq). The reaction mixture was stirred at 25° C. for 5 h. The reaction mixture was concentrated under reduced pressure to afford N-((1S)-(4,4-difluorocyclohexyl)(2-(((3R,5R)-2-oxo-5-(trifluoromethyl)piperidin-3-yl)methyl)-3-(5-oxa-2-azaspiro[3.4]octan-7-yl)imidazo[1,2-b][1,2,4]triazin-6-yl)methyl)-1-ethyl-1H-pyrazole-5-carboxamide (40.0 mg, 58.8 μmol, 91.7% yield) as a yellow solid.

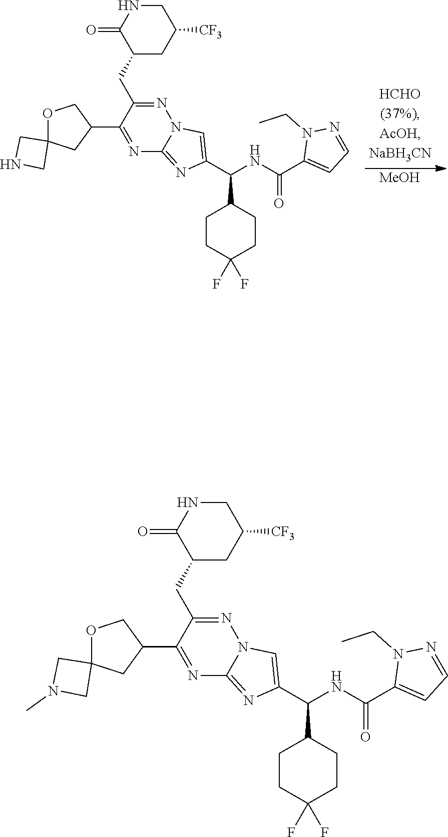 [SMILES: "MeOH" ; CCn1nccc1C(=O)N[C@H](c1cn2nc(C[C@H]3C[C@@H](C(F)(F)F)CNC3=O)c(C3COC4(CNC4)C3)nc2n1)C1CCC(F)(F)CC1 ; CCn1nccc1C(=O)N[C@H](c1cn2nc(C[C@H]3C[C@@H](C(F)(F)F)CNC3=O)c(C3COC4(C3)CN(C)C4)nc2n1)C1CCC(F)(F)CC1]

To a solution of N-((1S)-(4,4-difluorocyclohexyl)(2-(((3R,5R)-2-oxo-5-(trifluoromethyl)piperidin-3-yl)methyl)-3-(5-oxa-2-azaspiro[3.4]octan-7-yl)imidazo[1,2-b][1,2,4]triazin-6-yl)methyl)-1-ethyl-1H-pyrazole-5-carboxamide (40.0 mg, 58.8 μmol, 1.00 eq) in MeOH (1.00 mL) was added AcOH (21.2 mg, 353 μmol, 20.2 μL, 6.00 eq), NaBH₃CN (12.9 mg, 205 μmol, 3.50 eq) and formaldehyde (1.09 g, 13.4 mmol, 1.00 mL, 37.0% purity). The reaction mixture was stirred at 25° C. for 0.5 h. The reaction mixture was concentrated under reduced pressure to give a residue which was purified by Prep-HPLC (neutral condition; column: Waters Xbridge 150 mm×25 mm, 10 um; mobile phase: [water (NH₄HCO₃)—ACN]; gradient: 35%-55% B over 8 min) to afford N-((1S)-(4,4-difluorocyclohexyl)(3-(2-methyl-5-oxa-2-azaspiro[3.4]octan-7-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (19.26 mg, 27.4 μmol, 46.5% yield, 98.7% purity) as a white solid.

### Example 214. Preparation of N-((1S)-(4,4-difluorocyclohexyl)(2-(((3R,5R)-2-oxo-5-(trifluoromethyl)piperidin-3-yl)methyl)-3-(4,5,6,7-tetrahydropyrazolo[1,5-a]pyridin-5-yl)imidazo[1,2-b][1,2,4]triazin-6-yl)methyl)-1-ethyl-1H-pyrazole-5-carboxamide (Compound 358)

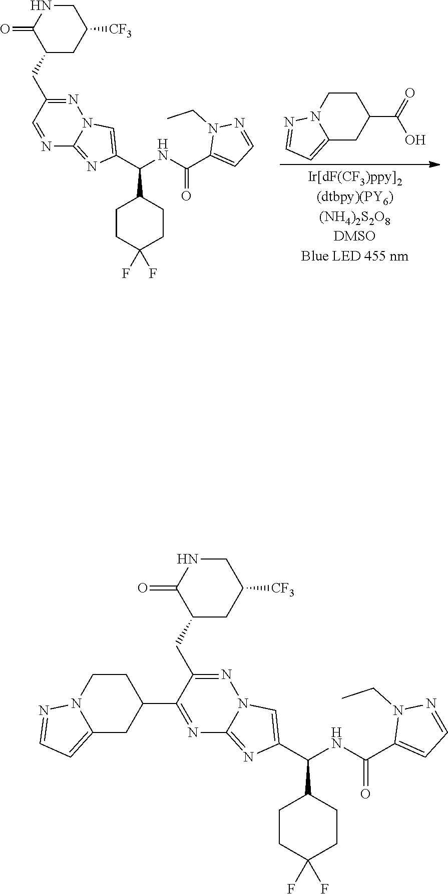

A mixture of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq), 4,5,6,7-tetrahydropyrazolo[1,5-a]pyridine-5-carboxylic acid (58.4 mg, 351 μmol, 2.00 eq), Ir[dF(CF₃)ppy]₂(dtbpy)(PF₆) (1.97 mg, 1.76 μmol, 0.010 eq), ammonium persulfate (80.2 mg, 351 μmol, 76.4 μL, 2.00 eq) in DMSO (2.00 mL) was degassed and purged with N₂. The reaction mixture was stirred at 25° C. for 2 h and irradiated with a 455 nm blue LED. The reaction mixture was partitioned between water and ethyl acetate. The organic phase was separated, washed with brine, dried over anhydrous sodium sulfate, filtered and concentrated under reduced pressure to give a residue, which was purified by Prep-TLC (SiO₂, Ethyl acetate:Methanol=5:1) and further purified by Prep-HPLC (FA condition: column: Phenomenex luna C18 150 mm×25 mm, 10 um; mobile phase: [water (FA)—ACN]; gradient: 40%-64% B over 8 min) to afford N-((1S)-(4,4-difluorocyclohexyl)(2-(((3R,5R)-2-oxo-5-(trifluoromethyl)piperidin-3-yl)methyl)-3-(4,5,6,7-tetrahydropyrazolo[1,5-a]pyridin-5-yl)imidazo[1,2-b][1,2,4]triazin-6-yl)methyl)-1-ethyl-1H-pyrazole-5-carboxamide (21.53 mg, 29.6 μmol, 16.8% yield, 94.8% purity) as a light yellow solid.

### Example 215. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-(dimethylamino)ethyl)-1H-pyrazole-5-carboxamide (Compound 362)

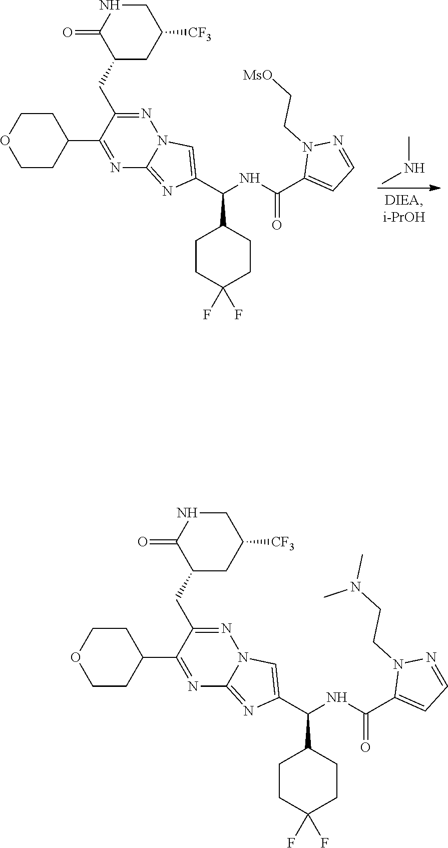

2-(5-(((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamoyl)-1H-pyrazol-1-yl)ethyl methanesulfonate (70.0 mg, 93.7 μmol, 1.00 eq), dimethylamine (2 M, 140 μL, 3.00 eq) and DIEA (36.3 mg, 281 μmol, 48.9 μL, 3.00 eq) were taken up into a microwave tube in i-PrOH (3.00 mL). The sealed tube was heated at 100° C. for 2 h under microwave irradiation. The reaction mixture was concentrated under reduced pressure to give a residue which was purified by Prep-HPLC (FA condition: column: Phenomenex luna C18 150 mm×25 mm, 10 um; mobile phase: [water (FA)—ACN]; gradient: 22%-42% B over 10 min) and further purified by Prep-HPLC (Neu condition: column: Waters Xbridge 150 mm×25 mm, 10 um; mobile phase: [water (NH₄HCO₃)—ACN]; gradient: 30%-50% B over 8 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-(dimethylamino)ethyl)-1H-pyrazole-5-carboxamide (25.54 mg, 34.9 μmol, 37.3% yield, 95.3% purity) as a yellow solid.

### Example 216. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-(2-(trifluoromethyl)pyrrolidin-1-yl)ethyl)-1H-pyrazole-5-carboxamide (Compound 430)

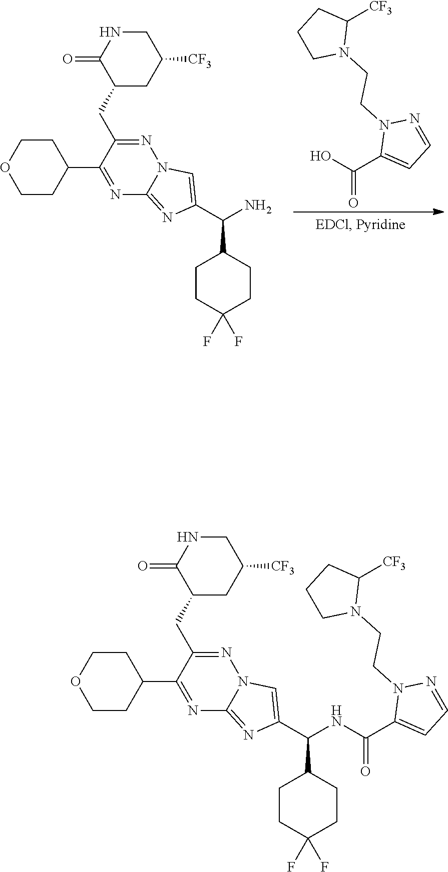 [SMILES: N[C@H](c1cn2nc(C[C@H]3C[C@@H](C(F)(F)F)CNC3=O)c(C3CCOCC3)nc2n1)C1CCC(F)(F)CC1 ; O=C(O)c1ccnn1CCN1CCCC1C(F)(F)F ; O=C(N[C@H](c1cn2nc(C[C@H]3C[C@@H](C(F)(F)F)CNC3=O)c(C3CCOCC3)nc2n1)C1CCC(F)(F)CC1)c1ccnn1CCN1CCCC1C(F)(F)F]

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-(2-(trifluoromethyl)pyrrolidin-1-yl)ethyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (60.0 mg, 113 μmol, 1.00 eq), 1-(2-(2-(trifluoromethyl)pyrrolidin-1-yl)ethyl)-1H-pyrazole-5-carboxylic acid (60.0 mg, 216 μmol, 1.91 eq) and was purified by Prep-HPLC (FA condition: column: Phenomenex luna C18 150 mm×25 mm, 10 um; mobile phase: [water (FA)—ACN]; gradient: 52%-76% B over 8 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-(2-(trifluoromethyl)pyrrolidin-1-yl)ethyl)-1H-pyrazole-5-carboxamide (55.51 mg, 67.7 μmol, 59.9% yield, 96.4% purity) as a white solid.

### Example 217. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide (Compound 363)

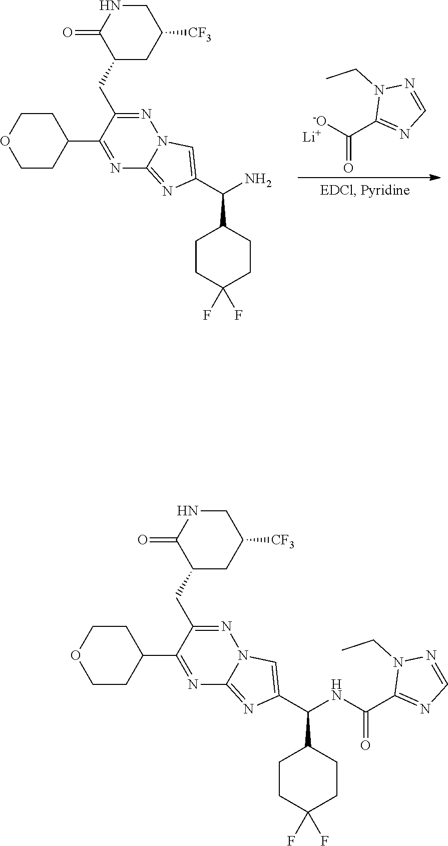

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (60.0 mg, 113 μmol, 1.00 eq), lithium 1-ethyl-1H-1,2,4-triazole-5-carboxylate (39.6 mg, 282 μmol, 2.50 eq) and was purified by prep-TLC (SiO₂, dichloromethane:methanol) and further purified by Prep-HPLC (FA condition; column: Phenomenex luna C18 150 mm×25 mm, 10 um; mobile phase: [water (FA)—ACN]; gradient: 48%-68% B over 10 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide (18.97 mg, 27.2 μmol, 24.0% yield, 93.8% purity) as a white solid.

### Example 218. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,3-triazole-5-carboxamide (Compound 364)

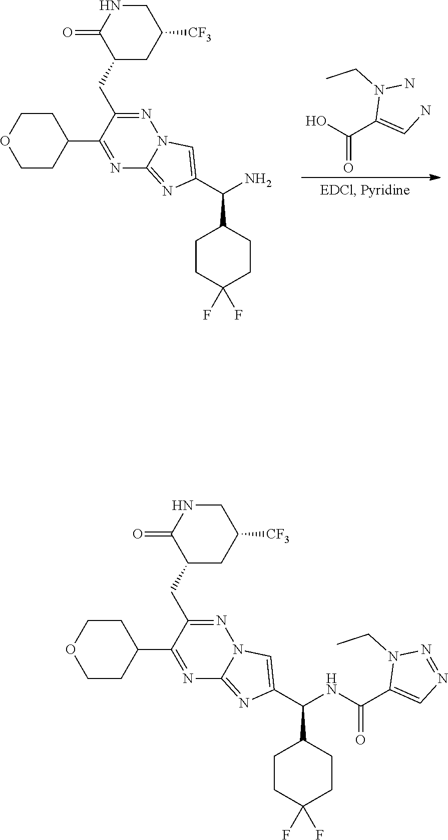

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,3-triazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 1-ethyl-1H-1,2,3-triazole-5-carboxylic acid (26.6 mg, 188 μmol, 2.00 eq) and was purified by Prep-HPLC (FA condition; column: Phenomenex luna C18 150 mm×25 mm, 10 um; mobile phase: [water (FA)—ACN]; gradient: 42%-66% B over 8 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,3-triazole-5-carboxamide (52.4 mg, 75.4 μmol, 80.0% yield, 94.1% purity) as a white solid.

### Example 219. Preparation of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (Compound 401 and 403)

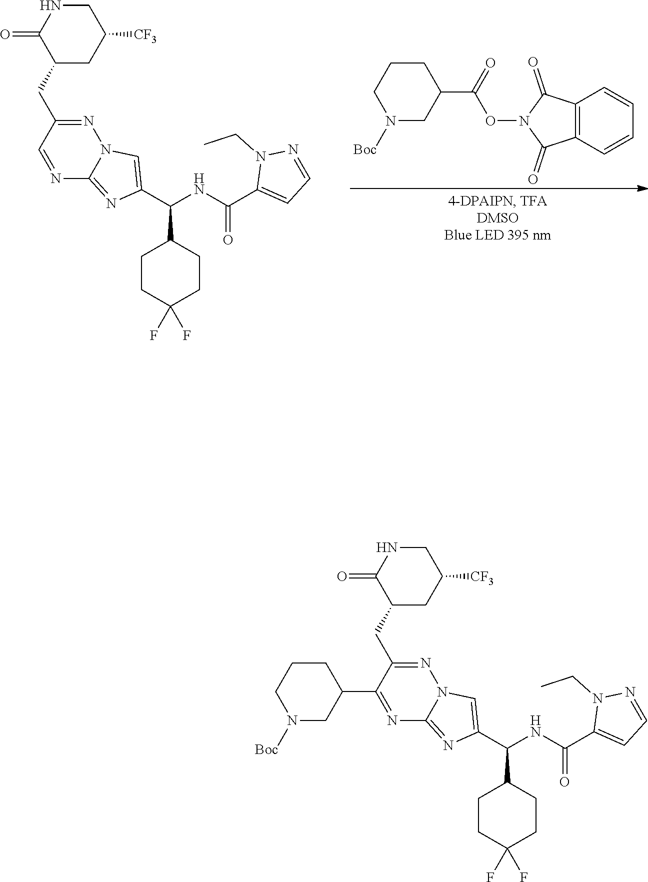 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCCC(C(=O)ON2C(=O)c3ccccc3C2=O)C1 ; CCn1nccc1C(=O)N[C@H](c1cn2nc(C[C@H]3C[C@@H](C(F)(F)F)CNC3=O)c(C3CCCN(C(=O)OC(C)(C)C)C3)nc2n1)C1CCC(F)(F)CC1]

Tert-butyl 3-(6-((S)-(4,4-difluorocyclohexyl)(1-ethyl-H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (400 mg, 703 μmol, 1.00 eq), 1-(tert-butyl) 3-(1,3-dioxoisoindolin-2-yl) piperidine-1,3-dicarboxylate (300 mg, 801 μmol, 1.14 eq) and was purified by Prep-HPLC (column: Phenomenex luna C18 150 mm×25 mm, 10 um; mobile phase: [water (FA)—ACN]; gradient: 51%-810% B over 10 min) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (250 mg, 332 μmol, 47.2% yield) as a white solid.

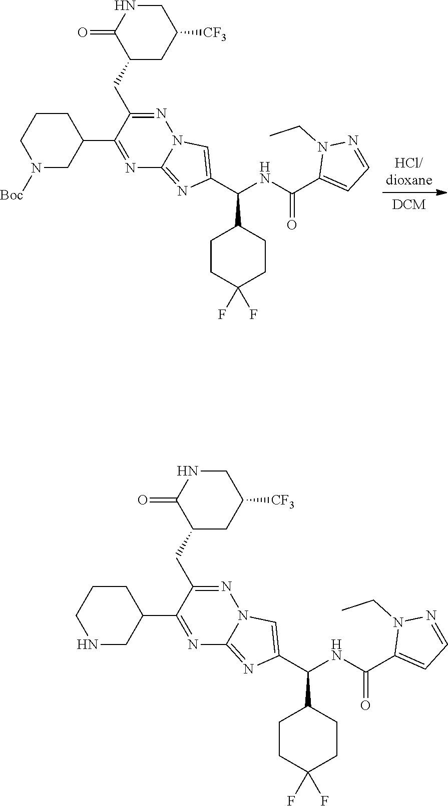 [SMILES: CCn1nccc1C(=O)N[C@H](c1cn2nc(C[C@H]3C[C@@H](C(F)(F)F)CNC3=O)c(C3CCCN(C(=O)OC(C)(C)C)C3)nc2n1)C1CCC(F)(F)CC1 ; CCn1nccc1C(=O)N[C@H](c1cn2nc(C[C@H]3C[C@@H](C(F)(F)F)CNC3=O)c(C3CCCNC3)nc2n1)C1CCC(F)(F)CC1]

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperidine-1-carboxylate (245 mg, 325 μmol, 1.00 eq) in dichloromethane (1.50 mL) was added HCl/dioxane (12 M, 27.1 μL, 1.00 eq). The reaction mixture was stirred at 25° C. for 0.5 h. The reaction mixture was concentrated under reduced pressure to afford N-((1S)-(4,4-difluorocyclohexyl)(2-(((3R,5R)-2-oxo-5-(trifluoromethyl)piperidin-3-yl)methyl)-3-(piperidin-3-yl)imidazo[1,2-b][1,2,4]triazin-6-yl)methyl)-1-ethyl-1H-pyrazole-5-carboxamide (130 mg, 199 mol, 61.2% yield) as a white solid.

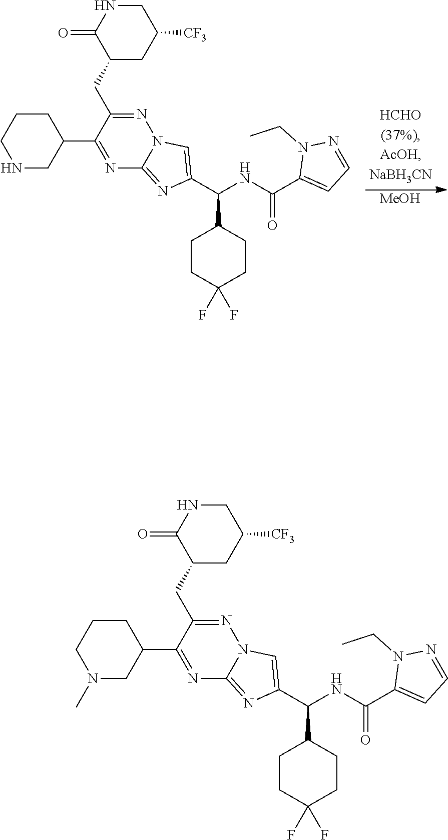 [SMILES: "MeOH" ; CCn1nccc1C(=O)N[C@H](c1cn2nc(C[C@H]3C[C@@H](C(F)(F)F)CNC3=O)c(C3CCCNC3)nc2n1)C1CCC(F)(F)CC1 ; CCn1nccc1C(=O)N[C@H](c1cn2nc(C[C@H]3C[C@@H](C(F)(F)F)CNC3=O)c(C3CCCN(C)C3)nc2n1)C1CCC(F)(F)CC1]

To a solution of N-((1S)-(4,4-difluorocyclohexyl)(2-(((3R,5R)-2-oxo-5-(trifluoromethyl)piperidin-3-yl)methyl)-3-(piperidin-3-yl)imidazo[1,2-b][1,2,4]triazin-6-yl)methyl)-1-ethyl-1H-pyrazole-5-carboxamide (60.0 mg, 87.1 μmol, 1.00 eq, HCl salt) in methanol (1.00 mL) was added acetic acid (31.4 mg, 523 μmol, 29.9 μL, 6.00 eq), NaBH₃CN (19.1 mg, 305 μmol, 3.50 eq) and formaldehyde (1.09 g, 13.4 mmol, 1.00 mL, 37% purity, 154 eq). The reaction mixture was stirred at 25° C. for 1 h. The reaction mixture was reduced under pressure to give a residue which was purified by Prep-HPLC (column: Phenomenex luna C18 150 mm×25 mm, 10 um; mobile phase: [water (FA)—ACN]; gradient: 22%-42% B over 10 min) to afford N-((1S)-(4,4-difluorocyclohexyl)(3-(1-methylpiperidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide.

Compound 401 was isolated as the first eluting, single stereoisomer (12.28 mg, 17.8 μmol, 20.5% yield, 96.9% purity) and was obtained as a white solid.

Compound 403 was isolated as the second eluting, single stereoisomer (10.45 mg, 15.5 μmol, 17.8% yield, 99.2% purity) and was obtained as a white solid.

### Example 220. Preparation of N-((1S)-(4,4-difluorocyclohexyl)(3-(1-(2-methoxyethyl)piperidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 226)

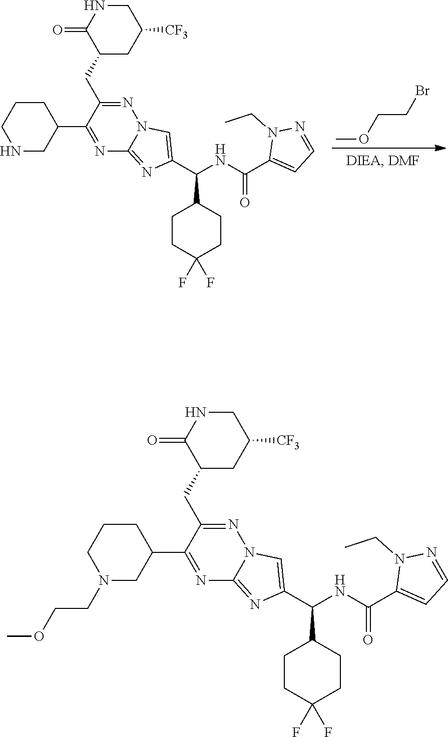

To a solution of N-((1S)-(4,4-difluorocyclohexyl)(2-(((3R,5R)-2-oxo-5-(trifluoromethyl)piperidin-3-yl)methyl)-3-(piperidin-3-yl)imidazo[1,2-b][1,2,4]triazin-6-yl)methyl)-1-ethyl-1H-pyrazole-5-carboxamide (60.0 mg, 92.0 μmol, 1.00 eq) and 1-bromo-2-methoxyethane (19.2 mg, 138 μmol, 12.9 μL, 1.50 eq) in DMF (2.00 mL) was added DIEA (35.0 mg, 276 μmol, 48 μL, 3.00 eq). The reaction mixture was stirred at 25° C. for 1 h. The reaction mixture was concentrated under reduced pressure to give a residue which was purified by Prep-HPLC (column: Phenomenex luna C18 150 mm×25 mm, 10 um; mobile phase: [water (FA)—ACN]; gradient: 20%-40% B over 10 min) to afford N-((1S)-(4,4-difluorocyclohexyl)(3-(1-(2-methoxyethyl)piperidin-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (34.37 mg, 46.8 μmol, 50.8% yield, 96.7% purity) as a white solid.

### Example 221. Preparation of N-((1S)-(4,4-difluorocyclohexyl)(3-(1-(2-methoxyethyl)piperidin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 369)

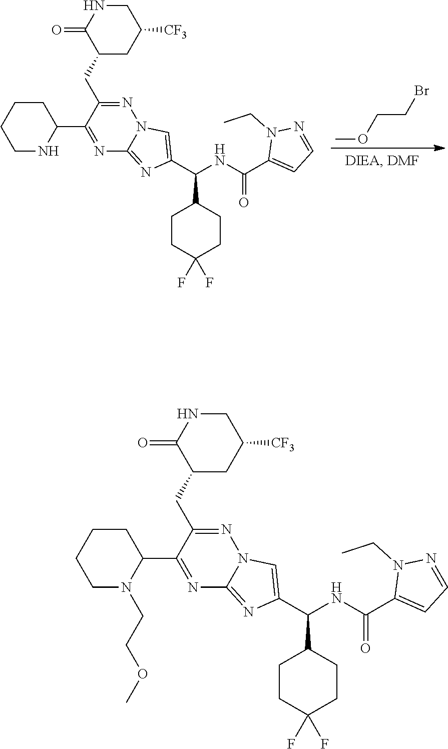

To a solution of N-((1S)-(4,4-difluorocyclohexyl)(2-(((3R,5R)-2-oxo-5-(trifluoromethyl)piperidin-3-yl)methyl)-3-(piperidin-2-yl)imidazo[1,2-b][1,2,4]triazin-6-yl)methyl)-1-ethyl-1H-pyrazole-5-carboxamide (50.0 mg, 76.7 μmol, 1.00 eq), 1-bromo-2-methoxyethane (21.3 mg, 153 μmol, 14.4 μL, 2.00 eq) in DMF (2.00 mL) was added DIEA (29.7 mg, 230 μmol, 40.0 μL, 3.00 eq). The reaction mixture was stirred at 25° C. for 24 h. The reaction mixture was concentrated under reduced pressure to give a residue which was purified by prep-HPLC (column: Phenomenex luna C18 150 mm×25 mm, 10 um; mobile phase: [water (FA)—ACN]; gradient: 20%-40% B over 10 min) to afford N-((1S)-(4,4-difluorocyclohexyl)(3-(1-(2-methoxyethyl)piperidin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (27.9 mg, 36.9 μmol, 48.1% yield, 93.7% purity) as a yellow solid.

### Example 222. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(2,2,2-trifluoroethoxy)-1,2,5-oxadiazole-3-carboxamide (Compound 372)

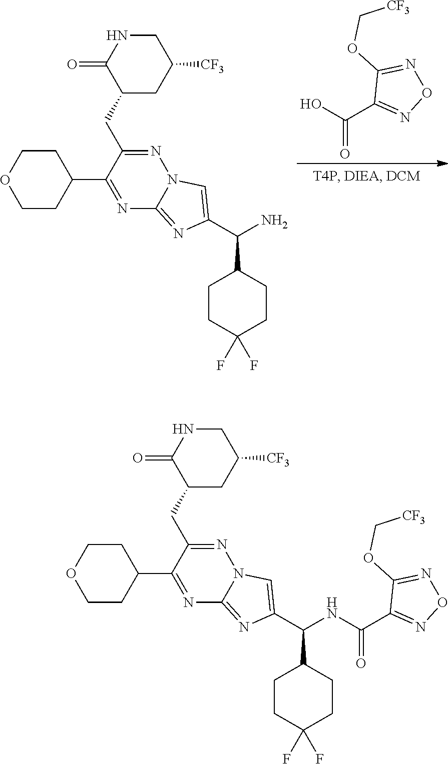

To a solution of (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (50.0 mg, 94.2 μmol, 1.00 eq), 4-(2,2,2-trifluoroethoxy)-1,2,5-oxadiazole-3-carboxylic acid (29.9 mg, 141 μmol, 1.50 eq) in Dichloromethane (1.00 mL) was added DIEA (60.9 mg, 471 μmol, 82.1 μL, 5.00 eq), T4P (135 mg, 188 μmol, 50.0% purity, 2.00 eq). The reaction mixture was stirred at 25° C. for 1 h. The reaction mixture was diluted with sat. aq. NaHCO₃ and water, extracted with dichloromethane. The combined organic layer was washed with brine, dried over anhydrous sodium sulfate, filtered and concentrated under reduced pressure to give a residue which was purified by Prep-TLC (SiO₂, Dichloromethane:Methanol=10:1) and further purified by Prep-HPLC (column: Waters Xbridge 150 mm×25 mm, 10 um; mobile phase: [water (NH₄HCO₃)—ACN]; gradient: 46%-66% B over 8 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(2,2,2-trifluoroethoxy)-1,2,5-oxadiazole-3-carboxamide (26.94 mg, 36.9 μmol, 39.2% yield, 99.4% purity) as a white solid.

### Example 223. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-((3-fluoroazetidin-1-yl)methyl)-1,2,5-oxadiazole-3-carboxamide (Compound 373)

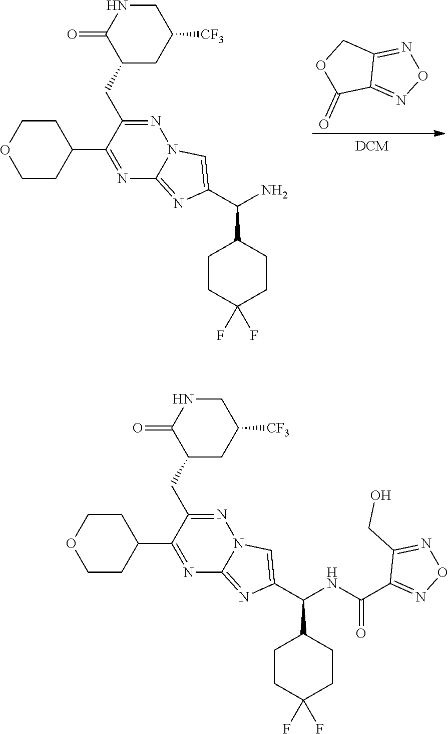

To a solution of (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (100 mg, 188 μmol, 1.00 eq) in dichloromethane (2.00 mL) was added 4H,6H-furo[3,4-c][1,2,5]oxadiazol-4-one (35.6 mg, 282 μmol, 1.50 eq). The reaction mixture was stirred at 25° C. for 1 h. The reaction mixture was diluted with water and extracted with ethyl acetate, dried over anhydrous sodium sulfate, filtered and concentrated under reduced pressure to give a residue which was purified by Prep-TLC (SiO₂, Dichloromethane:Methanol=10:1) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(hydroxymethyl)-1,2,5-oxadiazole-3-carboxamide (80.0 mg, 121 mol) as a yellow solid.

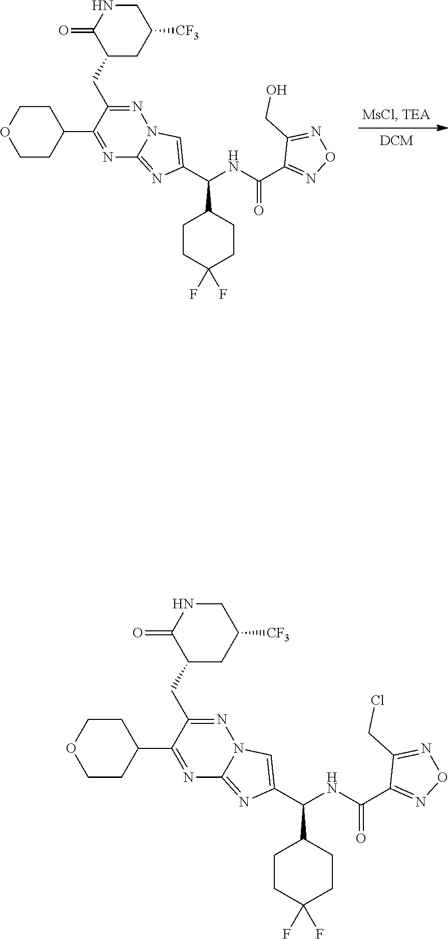

To a solution of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-(hydroxymethyl)-1,2,5-oxadiazole-3-carboxamide (40.0 mg, 60.9 μmol, 1.00 eq) in dichloromethane (1.00 mL) was added MsCl (34.8 mg, 304 μmol, 23.5 μL, 5.00 eq) and TEA (30.8 mg, 304 μmol, 42.4 μL, 5.00 eq) at 0° C. The reaction mixture was stirred at 25° C. for 1 h. The reaction mixture was diluted with water and extracted with dichloromethane, dried over anhydrous sodium sulfate, filtered and concentrated under reduced pressure to afford 4-(chloromethyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1,2,5-oxadiazole-3-carboxamide (40.0 mg, 54.4 μmol) as a yellow oil.

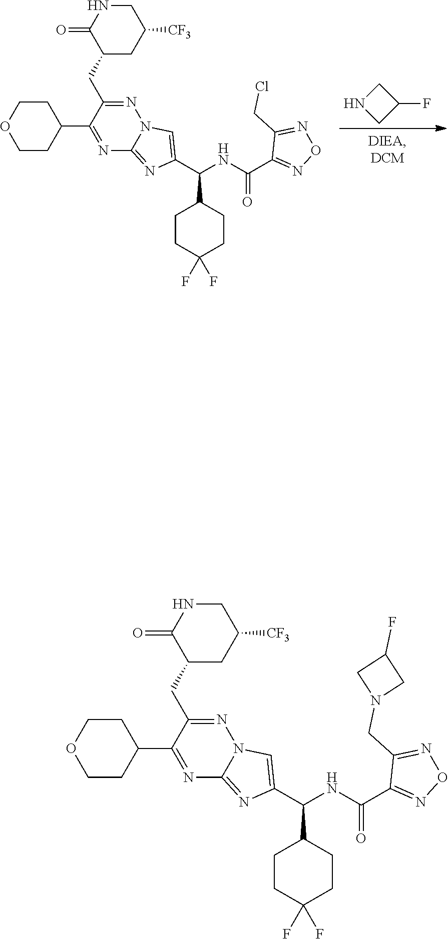

To a solution of 4-(chloromethyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1,2,5-oxadiazole-3-carboxamide (40.0 mg, 54.4 μmol, 1.00 eq) and 3-fluoroazetidine (18.2 mg, 163 μmol, 3.00 eq, HCl salt) in dichloromethane (1.00 mL) was added DIEA (35.1 mg, 272 μmol, 47.4 μL, 5.00 eq). The reaction mixture was stirred at 25° C. for 12 h. The reaction mixture was diluted with water and extracted with ethyl acetate, dried over anhydrous sodium sulfate, filtered and concentrated under reduced pressure to give a residue which was purified by Prep-HPLC (FA condition; column: Phenomenex luna C18 150 mm×25 mm, 10 um; mobile phase: [water (FA)—ACN]; gradient: 28%-46% B over 9 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-4-((3-fluoroazetidin-1-yl)methyl)-1,2,5-oxadiazole-3-carboxamide (95.2% purity) as a white solid.

### Example 224. Preparation of N-((1S)-(4,4-difluorocyclohexyl)(3-(4-methylmorpholin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 376 and 382)

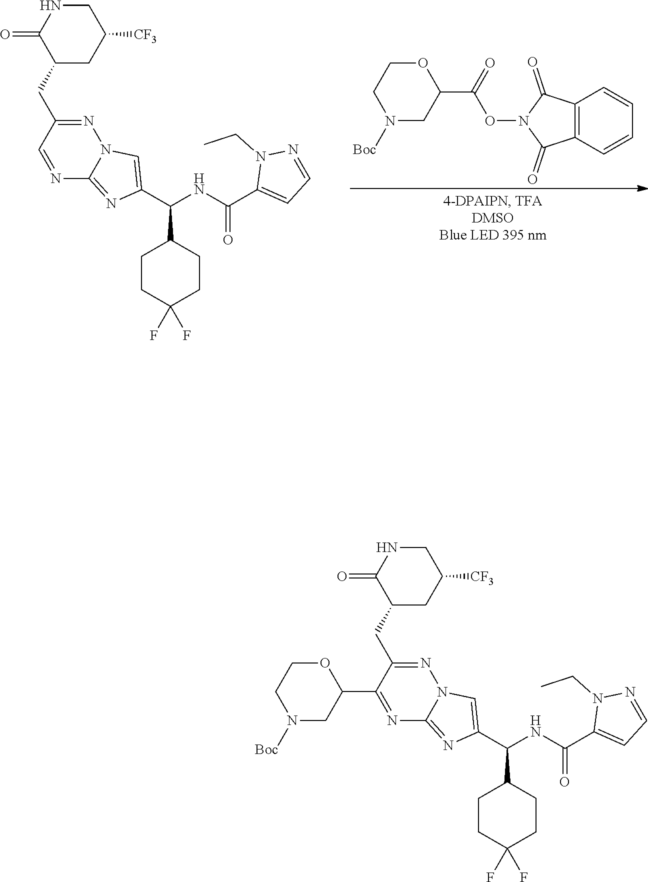 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCOC(C(=O)ON2C(=O)c3ccccc3C2=O)C1 ; CCn1nccc1C(=O)N[C@H](c1cn2nc(C[C@H]3C[C@@H](C(F)(F)F)CNC3=O)c(C3CN(C(=O)OC(C)(C)C)CCO3)nc2n1)C1CCC(F)(F)CC1]

Tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate was synthesized following the General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (200 mg, 351 μmol, 1.00 eq), 4-(tert-butyl) 2-(1,3-dioxoisoindolin-2-yl) morpholine-2,4-dicarboxylate (264 mg, 703 μmol, 2.00 eq) and was purified by Prep-HPLC (column: Phenomenex luna C18 150 mm×25 mm, 10 um; mobile phase: [water (FA)—ACN]; gradient: 45%-75% B over 10 min) to afford tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate (130 mg, 172 μmol, 49.0% yield) as a yellow solid.

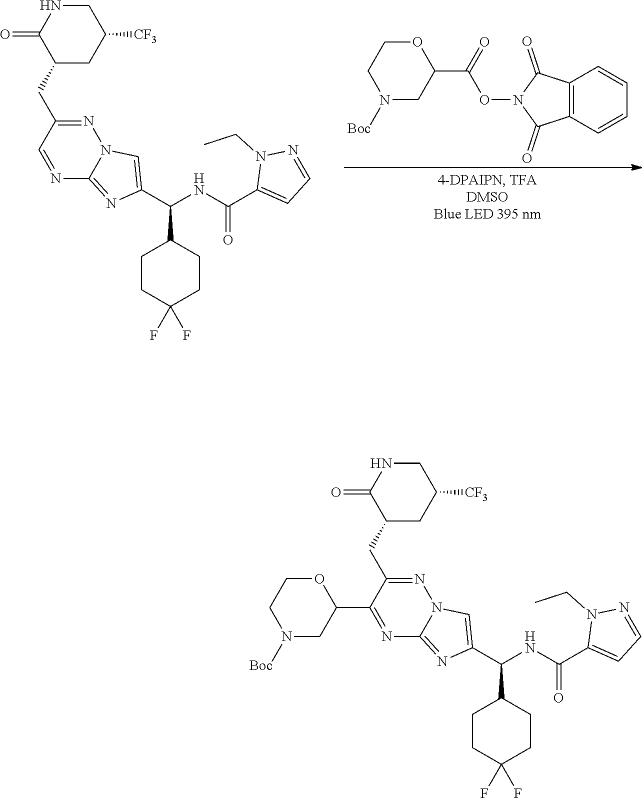

To a solution of tert-butyl 2-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)morpholine-4-carboxylate (100 mg, 132 μmol, 1.00 eq) in DCM (3.00 mL) was added ZnBr₂ (149 mg, 663 μmol, 33.2 μL, 5.00 eq). The reaction mixture was stirred at 25° C. for 24 h. The reaction mixture was concentrated under reduced pressure to afford N-((1S)-(4,4-difluorocyclohexyl)(3-(morpholin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (86.0 mg, 131 μmol, 99.1% yield) as a yellow solid.

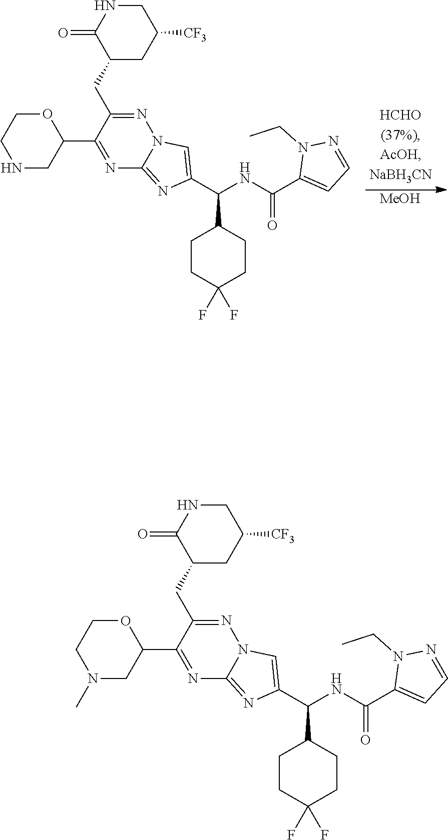 [SMILES: "MeOH" ; CCn1nccc1C(=O)N[C@H](c1cn2nc(C[C@H]3C[C@@H](C(F)(F)F)CNC3=O)c(C3CNCCO3)nc2n1)C1CCC(F)(F)CC1 ; CCn1nccc1C(=O)N[C@H](c1cn2nc(C[C@H]3C[C@@H](C(F)(F)F)CNC3=O)c(C3CN(C)CCO3)nc2n1)C1CCC(F)(F)CC1]

To a solution of N-((1S)-(4,4-difluorocyclohexyl)(3-(morpholin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (86.0 mg, 131 μmol, 1.00 eq) in methanol (1.50 mL) was added NaBH₃CN (24.8 mg, 394 μmol, 3.00 eq) and formaldehyde (1.39 g, 17.1 mmol, 1.27 mL, 37% purity, 130 eq). The reaction mixture was stirred at 25° C. for 0.5 h. The reaction mixture was reduced under pressure to give a residue which was purified by Prep-HPLC (column: Phenomenex luna C18 150 mm×25 mm, 10 um; mobile phase: [water (FA)—ACN]; gradient: 22%-42% B over 10 min) to afford N-((1S)-(4,4-difluorocyclohexyl)(3-(4-methylmorpholin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (75.0 mg, 112 μmol, 85.3% yield) as a yellow solid.

Compound 376 was isolated as the first eluting, single stereoisomer by chiral SFC purification (column: Daicel Chiralpak IK (250 mm×30 mm, 10 um); mobile phase: [CO₂-i-PrOH (0.1% NH₃H₂O)]; B %:45%, isocratic elution mode); (25.88 mg, 38.41 μmol, 34.2% yield, 99.1% purity) and was obtained as an orange solid.

Compound 382 was isolated as the second eluting, single stereoisomer by chiral SFC purification (column: Daicel Chiralpak IK (250 mm×30 mm, 10 um); mobile phase: [CO₂-i-PrOH (0.1% NH₃H₂O)]; B %:45%, isocratic elution mode); (27.27 mg, 39.86 μmol, 35.4% yield, 97.6% purity) and was obtained as an orange solid.

### Example 225. Preparation of N—((S)-cycloheptyl(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 377)

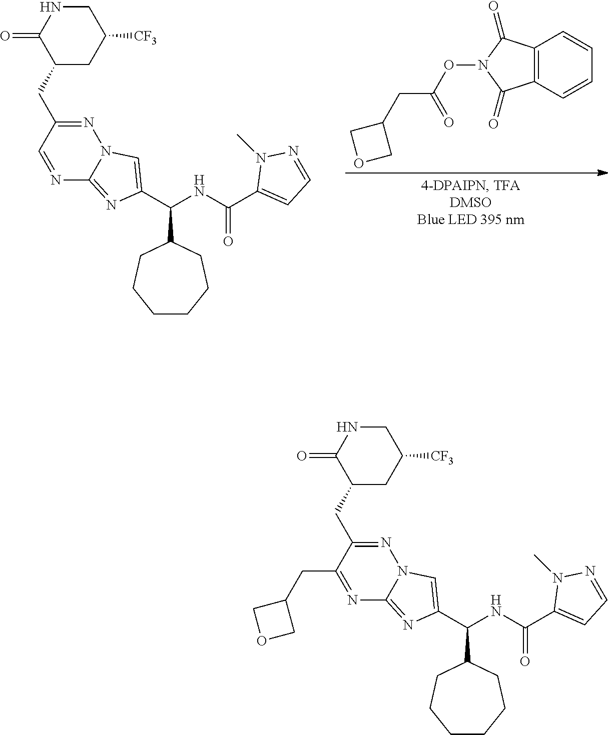 [SMILES: Cn1nccc1C(=O)N[C@H](c1cn2nc(C[C@H]3C[C@@H](C(F)(F)F)CNC3=O)cnc2n1)C1CCCCCC1 ; O=C(CC1COC1)ON1C(=O)c2ccccc2C1=O ; "4-DPAIPN, TFADMSOBlue LED 395 nm" ; Cn1nccc1C(=O)N[C@H](c1cn2nc(C[C@H]3C[C@@H](C(F)(F)F)CNC3=O)c(CC3COC3)nc2n1)C1CCCCCC1]

N—((S)-cycloheptyl(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide was synthesized following the General Procedure 7 with N—((S)-cycloheptyl(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (100 mg, 187 μmol, 1.00 eq), 1,3-dioxoisoindolin-2-yl 2-(oxetan-3-yl)acetate (98.1 mg, 375 μmol, 2.00 eq) and was purified by Prep-TLC (SiO₂, Ethyl acetate:Methanol=5:1) and then purified by Prep-HPLC (Neu condition: column: Waters Xbridge 150 mm×25 mm, 10 um; mobile phase: [water (NH₄HCO₃)—ACN]; gradient: 40%-60% B over 8 min) and further purified by Prep-SFC (column: Regis (S,S) Whelk—O1 (250 mm×25 mm, 10 um); mobile phase: [CO₂—ACN/i-PrOH (0.1% NH₃H₂O)]; B %: 55%, isocratic elution mode) to afford N—((S)-cycloheptyl(3-(oxetan-3-ylmethyl)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (11.72 mg, 18.1 μmol, 9.65% yield, 93.2% purity) as a yellow solid.

### Example 226. Preparation of N—((S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 233)

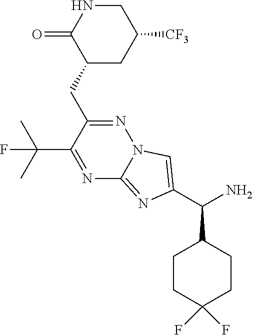 [SMILES: CC(C)(F)c1nc2nc([C@@H](N)C3CCC(F)(F)CC3)cn2nc1C[C@H]1C[C@@H](C(F)(F)F)CNC1=O]

(3R,5R)-3-((6-((S)-amino(4,4-difluorocyclohexyl)methyl)-3-(2-fluoropropan-2-yl)imidazo[1,2-b][1,2,4]triazin-2-yl)methyl)-5-(trifluoromethyl)piperidin-2-one was synthesized according to General Procedure 1, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate and 1,3-dioxoisoindolin-2-yl 2-fluoro-2-methylpropanoate, and General Procedure 3, employing benzyl ((S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)carbamate.

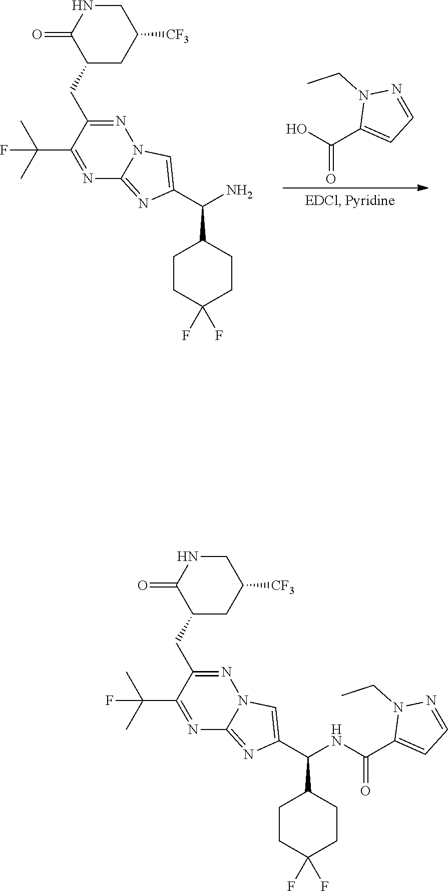

N—((S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(2-fluoropropan-2-yl)imidazo[1,2-b]l[1,2,4]triazin-2-yl)methyl)-5-(trifluoromethyl)piperidin-2-one (34.0 mg, 67.1 μmol, 1.00 eq), 1-ethyl-1H-pyrazole-5-carboxylic acid (16.0 mg, 114.1 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford N—((S)-(4,4-difluorocyclohexyl)(3-(2-fluoropropan-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (9.45 mg, 14.3 μmol, 21.3% yield, 95.0% purity) as a white solid.

### Example 227. Preparation of N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 234)

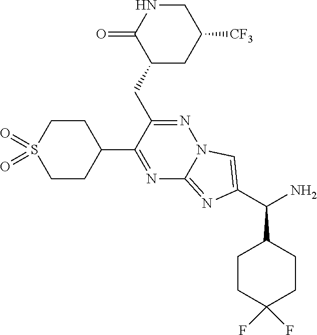 [SMILES: N[C@H](c1cn2nc(C[C@H]3C[C@@H](C(F)(F)F)CNC3=O)c(C3CCS(=O)(=O)CC3)nc2n1)C1CCC(F)(F)CC1]

(3R,5R)-3-((6-((S)-amino(4,4-difluorocyclohexyl)methyl)-3-(1,1-dioxidotetrahydro-2H-thiopyran-4-yl)imidazo[1,2-b][1,2,4]triazin-2-yl)methyl)-5-(trifluoromethyl)piperidin-2-one was synthesized according to General Procedure 1, employing (3R,5R)-3-((3-amino-1,2,4-triazin-6-yl)methyl)-5-(trifluoromethyl)piperidin-2-one and benzyl (S)-(3-bromo-1-(4,4-difluorocyclohexyl)-2-oxopropyl)carbamate, General Procedure 7, employing benzyl ((1S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate and 1,3-dioxoisoindolin-2-yl tetrahydro-2H-thiopyran-4-carboxylate 1,1-dioxide, and General Procedure 3, employing benzyl ((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)carbamate.

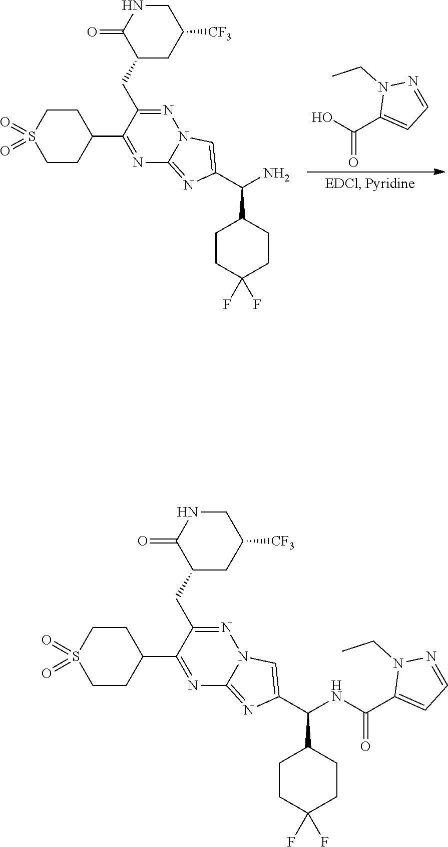

N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was synthesized following the General Procedure 6 with (3R,5R)-3-((6-((S)-amino(4,4-difluorocyclohexyl)methyl)-3-(1,1-dioxidotetrahydro-2H-thiopyran-4-yl)imidazo[1,2-b][1,2,4]triazin-2-yl)methyl)-5-(trifluoromethyl)piperidin-2-one (26.0 mg, 44.9 μmol, 1.00 eq), 1-ethyl-1H-pyrazole-5-carboxylic acid (10.7 mg, 76.4 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (11.41 mg, 15.0 μmol, 33.3% yield, 92.0% purity) as a white solid.

### Example 228. Preparation of 4-cyclopropyl-N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (Compound 243)

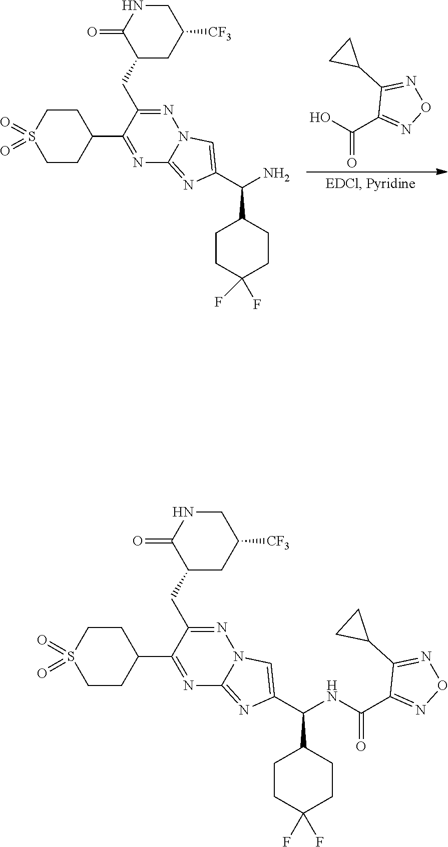 [SMILES: N[C@H](c1cn2nc(C[C@H]3C[C@@H](C(F)(F)F)CNC3=O)c(C3CCS(=O)(=O)CC3)nc2n1)C1CCC(F)(F)CC1 ; O=C(O)c1nonc1C1CC1 ; O=C(N[C@H](c1cn2nc(C[C@H]3C[C@@H](C(F)(F)F)CNC3=O)c(C3CCS(=O)(=O)CC3)nc2n1)C1CCC(F)(F)CC1)c1nonc1C1CC1]

4-Cyclopropyl-N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide was synthesized following the General Procedure 6 with (3R,5R)-3-((6-((S)-amino(4,4-difluorocyclohexyl)methyl)-3-(1,1-dioxidotetrahydro-2H-thiopyran-4-yl)imidazo[1,2-b][1,2,4]triazin-2-yl)methyl)-5-(trifluoromethyl)piperidin-2-one (20.0 mg, 34.6 mol, 1.00 eq), 4-cyclopropyl-1,2,5-oxadiazole-3-carboxylic acid (9.1 mg, 58.8 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford 4-cyclopropyl-N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1,2,5-oxadiazole-3-carboxamide (9.16 mg, 12.5 μmol, 36.3% yield, 97.8% purity) as a white solid.

### Example 229. Preparation of N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-1,2,4-triazole-5-carboxamide (Compound 248)

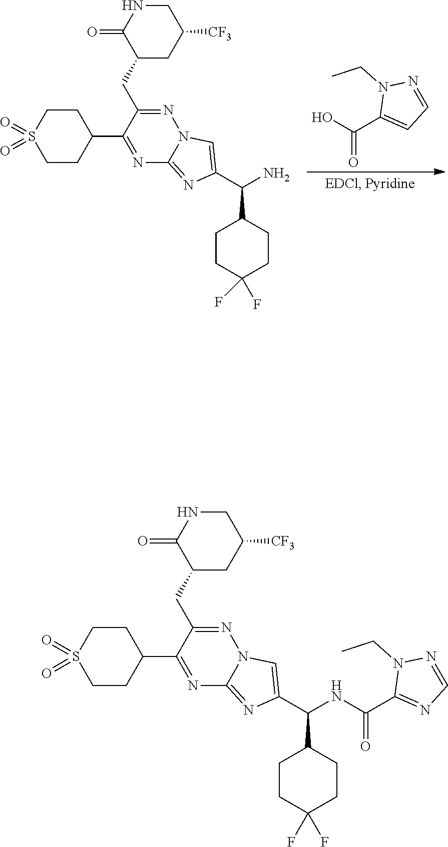

N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-1,2,4-triazole-5-carboxamide was synthesized following the General Procedure 6 with (3R,5R)-3-((6-((S)-amino(4,4-difluorocyclohexyl)methyl)-3-(1,1-dioxidotetrahydro-2H-thiopyran-4-yl)imidazo[1,2-b][1,2,4]triazin-2-yl)methyl)-5-(trifluoromethyl)piperidin-2-one (20.0 mg, 34.6 mol, 1.00 eq), 1-ethyl-1H-1,2,4-triazole-5-carboxylic acid (8.3 mg, 58.8 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford N—((S)-(4,4-difluorocyclohexyl)(3-(1,1-dioxidotetrahydro-2H-thiopyran-4-yl)-2-(((3R,5R)-2-oxo-5-(trifluoromethyl)piperidin-3-yl)methyl)imidazo[1,2-b][1,2,4]triazin-6-yl)methyl)-1-ethyl-1H-1,2,4-triazole-5-carboxamide (1.24 mg, 1.6 μmol, 4.5% yield, 88.0% purity) as a white solid.

### Example 230. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylpyrazine-2-carboxamide (Compound 246)

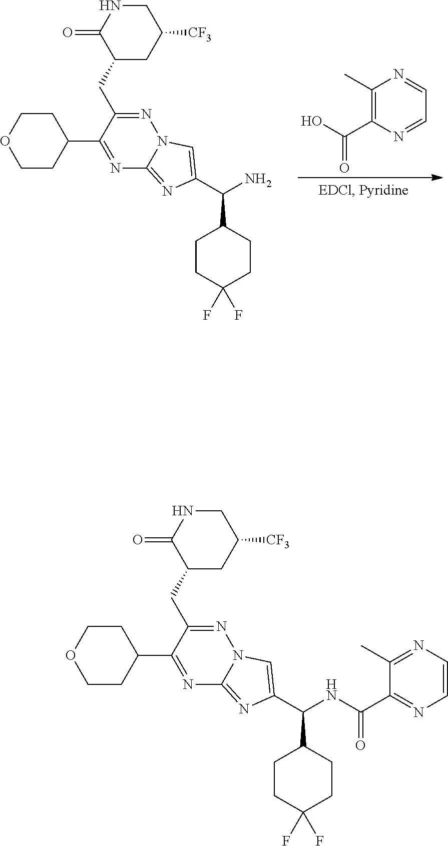

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylpyrazine-2-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (15.0 mg, 28.3 μmol, 1.00 eq), 3-methylpyrazine-2-carboxylic acid (6.6 mg, 48.1 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-3-methylpyrazine-2-carboxamide (11.4 mg, 15.9 μmol, 56.3% yield, 90.7% purity) as a white solid.

### Example 231. Preparation of 1-(tert-butyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (Compound 414)

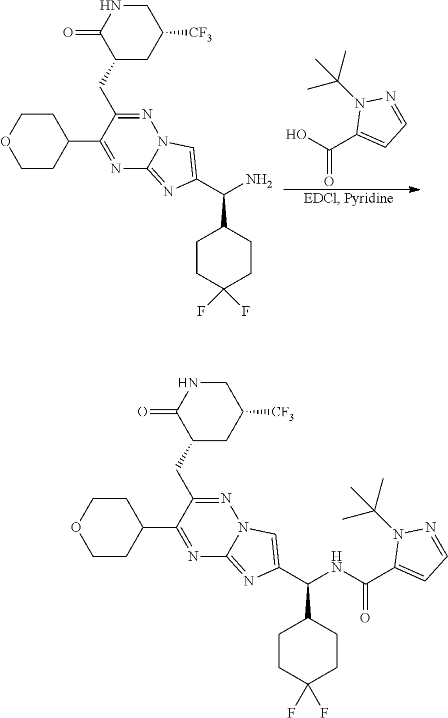

1-(Tert-buty)-N—((S)-(4,4-difluorocyclohexyl)(2-(((3R,5R)-2-oxo-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (10.0 mg, 18.8 μmol, 1.00 eq), 1-(tert-butyl)-1H-pyrazole-5-carboxylic acid (5.4 mg, 32.0 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford 1-(tert-butyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (8.40 mg, 11.3 μmol, 59.9% yield, 91.5% purity) as a white solid.

### Example 232. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-3-carboxamide (Compound 418)

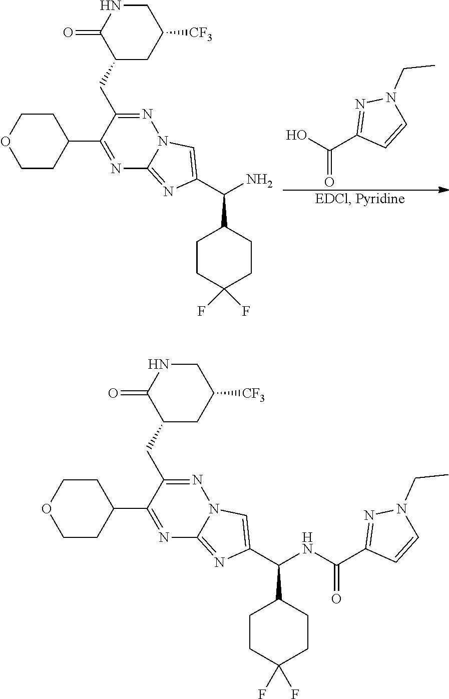

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-3-carboxamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (10.0 mg, 18.8 μmol, 1.00 eq), 1-ethyl-1H-pyrazole-3-carboxylic acid (4.5 mg, 32.0 μmol, 1.70 eq) and was purified by Prep-HPLC (column: Gemini C18 250 mm×21.2 mm, 5 um; mobile phase: [water (TFA)—ACN]; gradient: 10%-60% B over 30 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-pyrazole-3-carboxamide (7.75 mg, 11.5 μmol, 61.0% yield, 96.9% purity) as a white solid.

### Example 233. Preparation of N—((S)-(4,4-difluorocyclohexyl)(3-methyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 235)

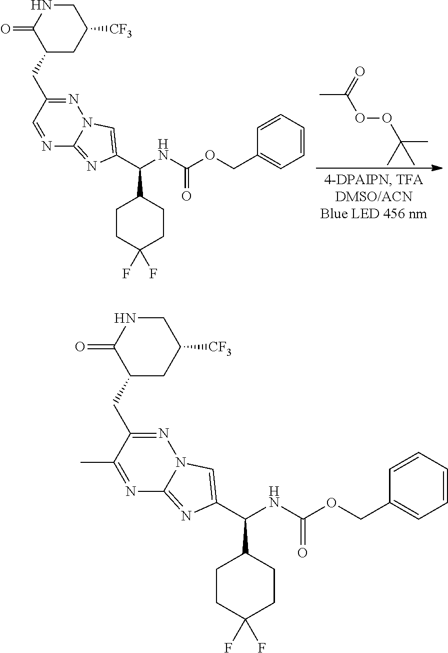

To a solution of benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (50.0 mg, 86.1 μmol, 1.00 eq) and tert-butyl ethaneperoxoate (113.8 mg, 431 μmol, 5.00 eq, 50 wt. % in odorless mineral spirits) in DMSO (0.80 mL) and acetonitrile (0.20 mL) was added 4-DPAIPN (2 mol %) (1.37 mg, 1.70 μmol, 0.020 eq) followed by TFA (98 mg, 861 μmol, 66 μL, 10.00 eq). The reaction mixture was degassed and purged with N₂. The reaction mixture was stirred at 25° C. for 18 h irradiated with a 456 nm blue LED. The reaction mixture concentrated under reduced pressure to give a residue which was purified by flash chromatography (SiO₂, dichloromethane:methanol=9:1) to afford benzyl ((S)-(4,4-difluorocyclohexyl)(3-methyl-2-(((3R,5R)-2-oxo-5-(trifluoromethyl)piperidin-3-yl)methyl)imidazo[1,2-b][1,2,4]triazin-6-yl)methyl)carbamate (18.0 mg, 30.3 μmol, 35.15% yield) as a yellow oil.

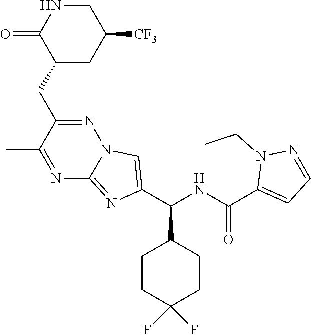 [SMILES: CCn1nccc1C(=O)N[C@H](c1cn2nc(C[C@H]3C[C@H](C(F)(F)F)CNC3=O)c(C)nc2n1)C1CCC(F)(F)CC1]

N—((S)-(4,4-difluorocyclohexyl)(3-methyl-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (2.23 mg, 3.3 μmol, 12.6% yield, 86% purity) was synthesized according to General Procedure 3, employing benzyl ((S)-(4,4-difluorocyclohexyl)(3-methyl-2-(((3R,5R)-2-oxo-5-(trifluoromethyl)piperidin-3-yl)methyl)imidazo[1,2-b][1,2,4]triazin-6-yl)methyl)carbamate, and General Procedure 6, employing (3R,5R)-3-((6-((S)-amino(4,4-difluorocyclohexyl)methyl)-3-methylimidazo[1,2-b][1,2,4]triazin-2-yl)methyl)-5-(trifluoromethyl)piperidin-2-one and 1-ethyl-1H-pyrazole-5-carboxylic acid.

### Example 234. General Scheme M

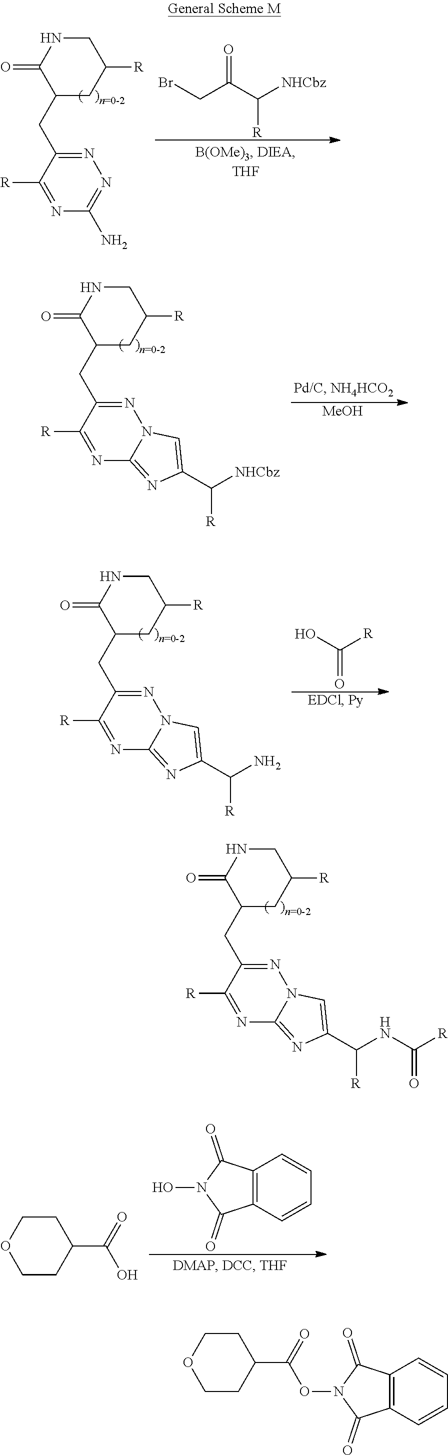

To a solution of tetrahydro-2H-pyran-4-carboxylic acid (30.0 g, 231 mmol, 1.00 eq) in THF (300 mL) was added DMAP (2.82 g, 23.1 mmol, 0.100 eq), DCC (57.1 g, 277 mmol, 56.0 mL, 1.20 eq), and 2-hydroxyisoindoline-1,3-dione (41.4 g, 254 mmol, 1.10 eq) at 0° C. The mixture was stirred at RT for 10 h. The reaction mixture was filtered with THF (500 mL), and the filtrate was concentrated under reduced pressure to give a residue. The residue was purified by column chromatography (SiO₂, Petroleum ether:Ethyl acetate=3:1) to afford 1,3-dioxoisoindolin-2-yl tetrahydro-2H-pyran-4-carboxylate (55.0 g, 200 mmol, 86.7% yield) as a yellow solid. ¹H NMR (400 MHz, CDCl₃) δ 7.88-7.83 (m, 2H), 7.81-7.73 (m, 2H), 4.08-3.92 (m, 2H), 3.61-3.44 (m, 2H), 3.08-2.89 (m, 1H), 2.14-1.88 (m, 4H).

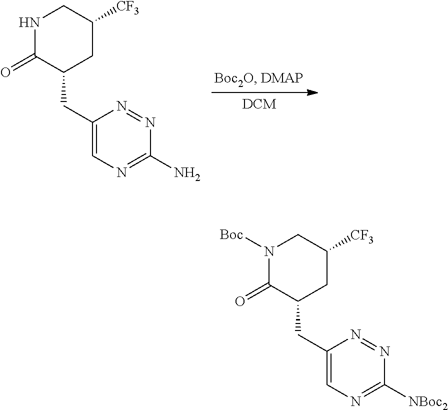

To a solution of (3R,5R)-3-((3-amino-1,2,4-triazin-6-yl)methyl)-5-(trifluoromethyl)piperidin-2-one (3.00 g, 10.9 mmol, 1.00 eq) in DCM (30.0 mL) was added Boc₂O (10.7 g, 49.0 mmol, 11.2 mL, 4.50 eq) and DMAP (266 mg, 2.18 mmol, 0.200 eq). The reaction mixture was stirred at RT for 12 h. The reaction mixture was partitioned between H₂O (50.0 mL) and DCM (50.0 mL*2). The organic phase was separated, washed with brine (50.0 mL), dried over anhydrous Na₂SO₄, filtered, and concentrated under reduced pressure to give a residue. The residue was purified by column chromatography (SiO₂, Petroleum ether:Ethyl acetate=100:1 to 1:1) to afford tert-butyl (3R,5R)-3-((3-(bis(tert-butoxycarbonyl)amino)-1,2,4-triazin-6-yl)methyl)-2-oxo-5-(trifluoromethyl)piperidine-1-carboxylate (5.00 g, 8.69 mmol, 79.7% yield) as a colorless oil. ¹H NMR (400 MHz, CDCl₃) δ 8.71 (s, 1H), 4.34 (dd, J₁=4.0 Hz, J₂=14.8 Hz, 1H), 3.66 (dd, J₁=6.0, J₂=14.8 Hz, 1H), 3.51 (dd, J₁=7.2, J₂=14.8 Hz, 1H), 3.39-3.25 (m, 1H), 3.13 (dd, J₁=5.6, J₂=14.8 Hz, 1H), 2.88-2.74 (m, 1H), 2.45-2.32 (m, 1H), 1.83-1.70 (m, 1H), 1.51 (s, 9H), 1.45 (s, 18H).

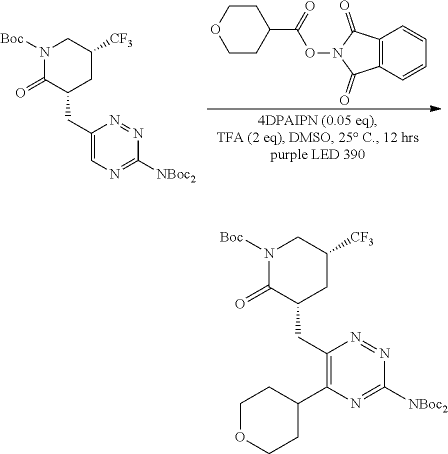

To a solution of tert-butyl (3R,5R)-3-((3-(bis(tert-butoxycarbonyl)amino)-1,2,4-triazin-6-yl)methyl)-2-oxo-5-(trifluoromethyl)piperidine-1-carboxylate (3.00 g, 5.21 mmol, 1.00 eq) and 1,3-dioxoisoindolin-2-yl tetrahydro-2H-pyran-4-carboxylate (2.87 g, 10.4 mmol, 2.00 eq) in DMSO (30.0 mL) was added 2, 4, 5, 6-tetrakis (N-phenylanilino) benzene-1, 3-dicarbonitrile (207 mg, 260 μmol, 0.0500 eq) and TFA (1.19 g, 10.4 mmol, 774 μL, 2.00 eq). The reaction mixture was stirred at RT for 12 h. The reaction mixture was partitioned between H₂O (50.0 mL) and EtOAc (50.0 mL×2). The organic phase was separated, washed with brine (50.0 mL), dried over anhydrous Na₂SO₄, filtered, and concentrated under reduced pressure to give a residue. The residue was purified by prep-HPLC (eluting with 55-85% ACN water (0.1% formic acid), Phenomenex luna C18 (250×70 mm, 10 um) to afford (3R,5R)-3-((3-amino-5-(tetrahydro-2H-pyran-4-yl)-1,2,4-triazin-6-yl)methyl)-5-(trifluoromethyl)piperidin-2-one (1.93 g, 2.93 mmol, 56.1% yield) was obtained as a yellow solid. LCMS [M+H]⁺=660.3 m/z.

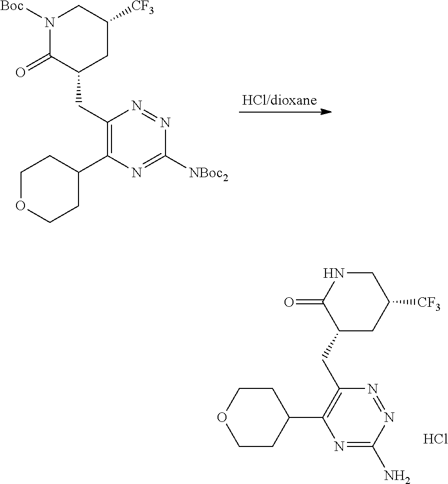

To a solution of tert-butyl (3R,5R)-3-((3-(bis(tert-butoxycarbonyl)amino)-5-(tetrahydro-2H-pyran-4-yl)-1,2,4-triazin-6-yl)methyl)-2-oxo-5-(trifluoromethyl)piperidine-1-carboxylate (1.93 g, 2.93 mmol, 1.00 eq) in HCl/dioxane (4.00 M, 20.0 mL, 27.3 eq). The mixture was stirred at 40° C. for 1 h. The reaction mixture was concentrated to afford (3R,5R)-3-((3-amino-5-(tetrahydro-2H-pyran-4-yl)-1,2,4-triazin-6-yl)methyl)-5-(trifluoromethyl)piperidin-2-one hydrochloride (1.00 g, crude) as a brown solid. LCMS [M+H]⁺=360.1 m/z.

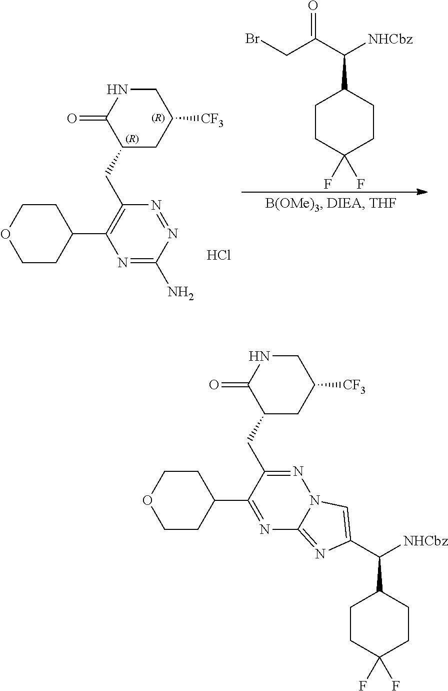

To a solution of (3R,5R)-3-((3-amino-5-(tetrahydro-2H-pyran-4-yl)-1,2,4-triazin-6-yl)methyl)-5-(trifluoromethyl)piperidin-2-one hydrochloride (1.00 g, 2.78 mmol, 1.00 eq) and benzyl (S)-(3-bromo-1-(4,4-difluorocyclohexyl)-2-oxopropyl)carbamate (1.69 g, 4.17 mmol, 1.50 eq) in THF (15.0 mL) was added DIEA (1.80 g, 13.9 mmol, 2.42 mL, 5.00 eq) and B(OMe)₃ (1.45 g, 13.9 mmol, 1.57 mL, 5.00 eq). The reaction mixture was stirred at 70° C. for 4 h. The reaction mixture was partitioned between H₂O (50.0 mL) and AtOAc (50.0 mL×2). The organic phase was separated, washed with brine (50.0 mL), dried over anhydrous Na₂SO₄, filtered, and concentrated under reduced pressure to give a residue. The residue was purified by column chromatography (SiO₂, DCM:MeOH=100:1 to 10:1) to afford benzyl ((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate (1.60 g, 2.41 mmol, 86.5% yield) as a brown solid. LCMS [M+H]⁺=665.4 m/z.

### Example 235. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-morpholinoethyl)-1H-pyrazole-5-carboxamide (Compound 378)

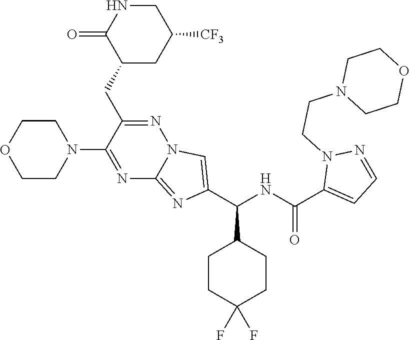 [SMILES: O=C(N[C@H](c1cn2nc(C[C@H]3C[C@@H](C(F)(F)F)CNC3=O)c(N3CCOCC3)nc2n1)C1CCC(F)(F)CC1)c1ccnn1CCN1CCOCC1]

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-morpholinoethyl)-1H-pyrazole-5-carboxamide was prepared according to General Procedure 6, employing (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (205 mg, 388 mol) and 1-(2-bromoethyl)-1H-pyrazole-5-carboxylic acid (170 mg, 776 mol) to afford 1-(2-bromoethyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide, which was converted to the title compound in the following procedure: 1-(2-bromoethyl)-N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-pyrazole-5-carboxamide (60.0 mg, 82.0 μmol, 1.00 eq), morpholine (14.2 mg, 164 μmol, 14.4 μL, 2.00 eq), and DIEA (21.2 mg, 164 μmol, 28.5 μL, 2.00 eq) were combined in a microwave reaction tube and diluted with i-PrOH (3.00 mL). The sealed tube was heated at 100° C. for 2 h under microwave. The reaction mixture was concentrated to give a residue. The residue was purified by prep-HPLC (eluting with 26%-46% water (0.1% formic acid) and ACN, YMC Triart C18 150×25 mm, 5 um) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-(2-morpholinoethyl)-1H-pyrazole-5-carboxamide (4.06 mg, 5.16 μmol, 6.29% yield) as a yellow solid.

### Example 236. Preparation of 1-ethyl-N—((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-1,2,4-triazole-5-carboxamide (Compound 383)

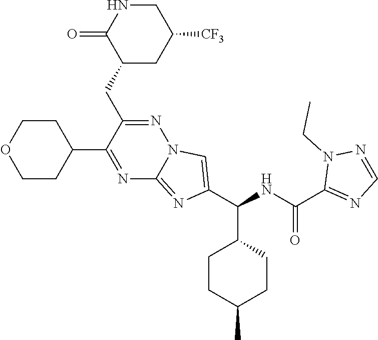 [SMILES: CCn1ncnc1C(=O)N[C@H](c1cn2nc(C[C@H]3C[C@@H](C(F)(F)F)CNC3=O)c(C3CCOCC3)nc2n1)[C@H]1CC[C@H](C)CC1]

1-ethyl-N—((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1H-1,2,4-triazole-5-carboxamide was prepared according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one (100 mg, 278 mol) and benzyl ((S)-3-bromo-1-((1r,4S)-4-methylcyclohexyl)-2-oxopropyl)carbamate (149 mg, 390 μmol), General Procedure 4, employing benzyl ((S)-((1r,4S)-4-methylcyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate (130 mg, 202 μmol), and General Procedure 6, employing (3R,5R)-3-((6-((S)-amino((1r,4S)-4-methylcyclohexyl)methyl)-3-(tetrahydro-2H-pyran-4-yl)imidazo[1,2-b][1,2,4]triazin-2-yl)methyl)-5-(trifluoromethyl)piperidin-2-one (80.0 mg, 157 mol) and lithium 1-ethyl-1H-1,2,4-triazole-5-carboxylate (46.6 mg, 315 μmol) to afford the title compound as a white solid (10.8 mg, 16.2 μmol, 10.3% yield).

### Example 237. Preparation of N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide (Compound 422)

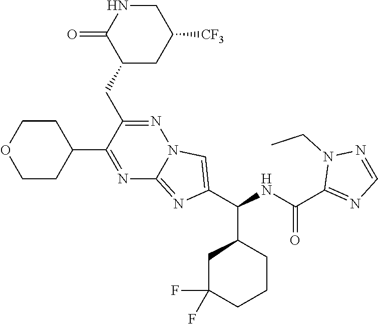 [SMILES: CCn1ncnc1C(=O)N[C@H](c1cn2nc(C[C@H]3C[C@@H](C(F)(F)F)CNC3=O)c(C3CCOCC3)nc2n1)[C@H]1CCCC(F)(F)C1]

N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide was prepared according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one (150 mg, 417 μmol) and benzyl ((S)-3-bromo-1-((S)-3,3-difluorocyclohexyl)-2-oxopropyl)carbamate (253 mg, 626 μmol), General Procedure 5, employing benzyl ((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate (150 mg, 225 μmol), and General Procedure 6, employing (3R,5R)-3-((6-((S)-amino((S)-3,3-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (35.0 mg, 65.9 μmol) and 1-ethyl-1H-1,2,4-triazole-5-carboxylic acid (14.5 mg, 98.9 mol) to afford the title compound (24.5 mg, 36.9 μmol, 55.9% yield) as a white solid.

### Example 238. Preparation of N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide (Compound 384)

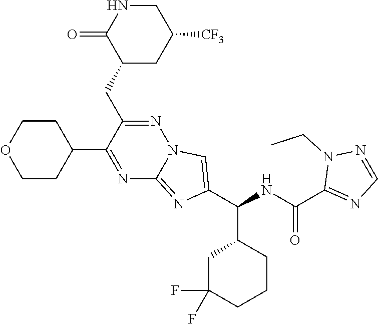 [SMILES: CCn1ncnc1C(=O)N[C@H](c1cn2nc(C[C@H]3C[C@@H](C(F)(F)F)CNC3=O)c(C3CCOCC3)nc2n1)[C@@H]1CCCC(F)(F)C1]

N—((S)—((S)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-1-ethyl-1H-1,2,4-triazole-5-carboxamide was prepared according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one (100 mg, 278 μmol) and benzyl ((S)-3-bromo-1-((R)-3,3-difluorocyclohexyl)-2-oxopropyl)carbamate (134 mg, 333 μmol), General Procedure 4, employing benzyl ((S)—((R)-3,3-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)carbamate (170 mg, 255 mol), and General Procedure 6, employing (3R,5R)-3-((6-((S)-amino((R)-3,3-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (90.0 mg, 169 μmol) and 1-ethyl-1H-1,2,4-triazole-5-carboxylic acid (59.8 mg, 424 μmol) to afford the title compound (18.49 mg, 27.3 μmol, 16.1% yield) as a white solid.

### Example 239. Preparation of N—((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-ethyl-1H-1,2,4-triazole-5-carboxamide (Compound 385)

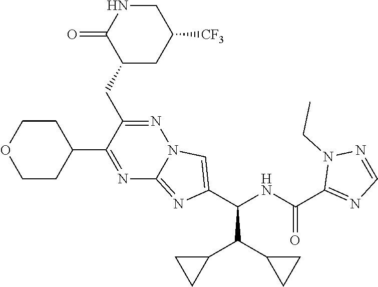 [SMILES: CCn1ncnc1C(=O)N[C@H](c1cn2nc(C[C@H]3C[C@@H](C(F)(F)F)CNC3=O)c(C3CCOCC3)nc2n1)C(C1CC1)C1CC1]

N—((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)-1-ethyl-1H-1,2,4-triazole-5-carboxamide was prepared according to General Procedure 3, employing (3R,5R)-3-((3-amino-5-(tetrahydro-2H-pyran-4-yl)-1,2,4-triazin-6-yl)methyl)-5-(trifluoromethyl)piperidin-2-one (100 mg, 278 μmol) and benzyl (S)-(4-bromo-1,1-dicyclopropyl-3-oxobutan-2-yl)carbamate (158 mg, 417 μmol), General Procedure 4, employing benzyl ((S)-2,2-dicyclopropyl-1-(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)ethyl)carbamate (130 mg, 202 μmol), and General Procedure 6, employing (3R,5R)-3-((6-((S)-1-amino-2,2-dicyclopropylethyl)-3-(tetrahydro-2H-pyran-4-yl)imidazo[1,2-b][1,2,4]triazin-2-yl)methyl)-5-(trifluoromethyl)piperidin-2-one (50.0 mg, 98.7 μmol) and 1-ethyl-1H-1,2,4-triazole-5-carboxylic acid (27.8 mg, 197 mol) to afford the title compound (10.9 mg, 17.3 μmol, 16.4% yield) as a white solid.

### Example 240. Preparation of N—((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-1,2,4-triazole-5-carboxamide (Compound 386)

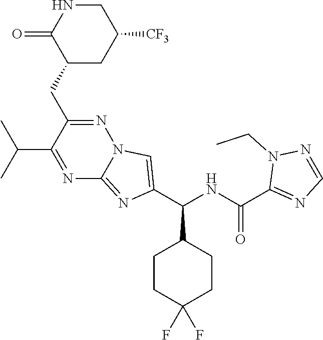 [SMILES: CCn1ncnc1C(=O)N[C@H](c1cn2nc(C[C@H]3C[C@@H](C(F)(F)F)CNC3=O)c(C(C)C)nc2n1)C1CCC(F)(F)CC1]

N—((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)-1-ethyl-1H-1,2,4-triazole-5-carboxamide was prepared according to General Procedure 7, employing benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (120 mg, 206 μmol) and 1,3-dioxoisoindolin-2-yl isobutyrate (96.4 mg, 413 μmol), General Procedure 4, employing benzyl ((S)-(4,4-difluorocyclohexyl)(3-isopropyl-2-(((3R,5R)-2-oxo-5-(trifluoromethyl)piperidin-3-yl)methyl)imidazo[1,2-b][1,2,4]triazin-6-yl)methyl)carbamate (80.0 mg, 128 mol), and General Procedure 6, employing (3R,5R)-3-((6-((S)-amino(4,4-difluorocyclohexyl)methyl)-3-isopropylimidazo[1,2-b][1,2,4]triazin-2-yl)methyl)-5-(trifluoromethyl)piperidin-2-one (40.0 mg, 81.8 μmol) and lithium 1-ethyl-1H-1,2,4-triazole-5-carboxylate (24.2 mg, 163 mol) to afford the title compound (25.3 mg, 41.0 μmol, 50.1% yield) as a white solid.

### Example 241. Preparation of N-((1S)-(4,4-difluorocyclohexyl)(3-(6,7-dihydro-5H-cyclopenta[d]pyrimidin-6-yl)-2-(((3R,5R)-2-oxo-5-(trifluoromethyl)pyneridin-3-yl)methyl)imidazo[1,2-b][1,2,4]triazin-6-yl)methyl)-1-ethyl-1H-pyrazole-5-carboxamide (Compound 400 and 402)

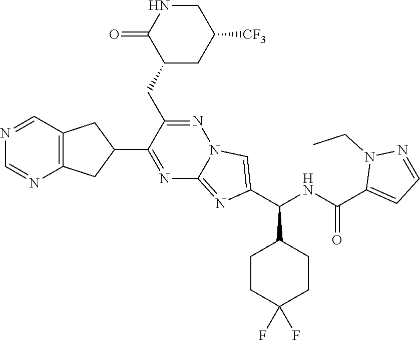 [SMILES: CCn1nccc1C(=O)N[C@H](c1cn2nc(C[C@H]3C[C@@H](C(F)(F)F)CNC3=O)c(C3Cc4cncnc4C3)nc2n1)C1CCC(F)(F)CC1]

N-((1S)-(4,4-difluorocyclohexyl)(3-(6,7-dihydro-5H-cyclopenta[d]pyrimidin-6-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide was prepared according to General Procedure 7, employing N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 mol) and 1,3-dioxoisoindolin-2-yl 6,7-dihydro-5H-cyclopenta[d]pyrimidine-6-carboxylate (54.4 mg, 175 mol) to afford the title compound as a mixture of stereoisomers, which were further purified by chiral prep-SFC (eluting with 30% ACN in water (0.1% formic acid), Daicel Chiralpak AD (250×30, 10 um)) to afford 26872 as the first eluting, single stereoisomer (13.9 mg, 19.4 μmol, 22.1% yield) as a white solid and 26875 as the second eluting, single stereoisomer (12.4 mg, 17.6 μmol, 20.0% yield) as a white solid.

### Example 242. Preparation of N—((S)-(4,4-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 282)

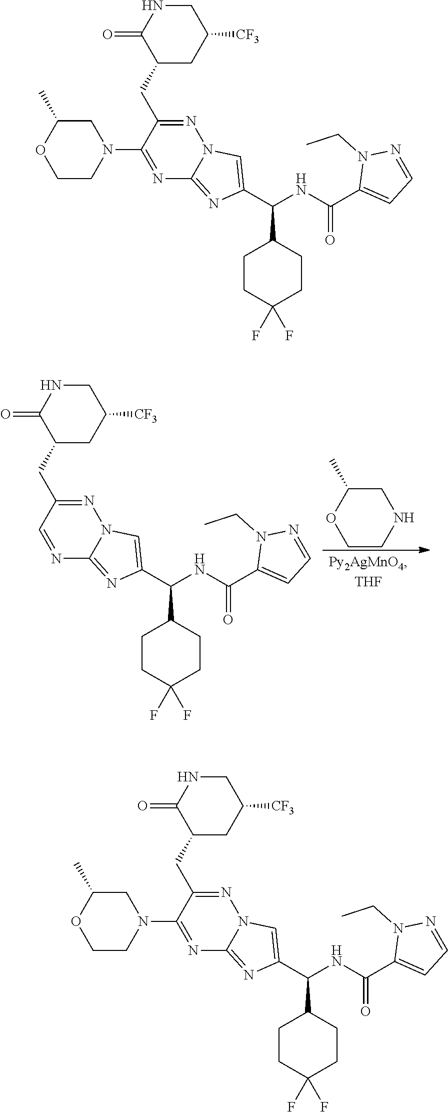

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with (R)-2-methylmorpholine (177 mg, 1.76 mmol, 20.0 eq) was carried out following General Procedure 8, using bis(pyridine)silver(I) permanganate (170 mg, 439 μmol, 5.00 eq) in THF (0.09 M) and purifying by Prep-HPLC (column: Phenomenex luna C18 150*25 mm*10 um; mobile phase: [water (FA)—ACN]; gradient: 30%-50% B over 10 min) to afford N—((S)-(4,4-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (34.29 mg, 51.25 μmol, 58% yield).

### Example 243. Preparation of N—((S)-(4,4-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 283)

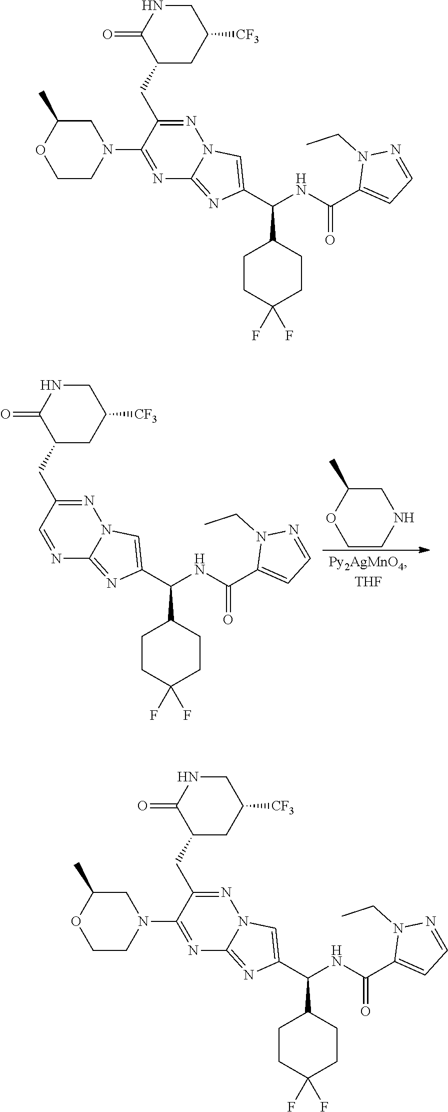

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with (S)-2-methylmorpholine (177 mg, 1.76 mmol, 20.0 eq) was carried out following General Procedure 8, using bis(pyridine)silver(I) permanganate (170 mg, 439 μmol, 5.00 eq) in THF (0.09 M) and purifying by Prep-HPLC (column: Phenomenex luna C18 150*25 mm*10 um; mobile phase: [water (FA)—ACN]; gradient: 30%-50% B over 10 min) to afford N—((S)-(4,4-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-11H-pyrazole-5-carboxamide (23.79 mg, 35.42 μmol, 40% yield).

### Example 244. Preparation of N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 296)

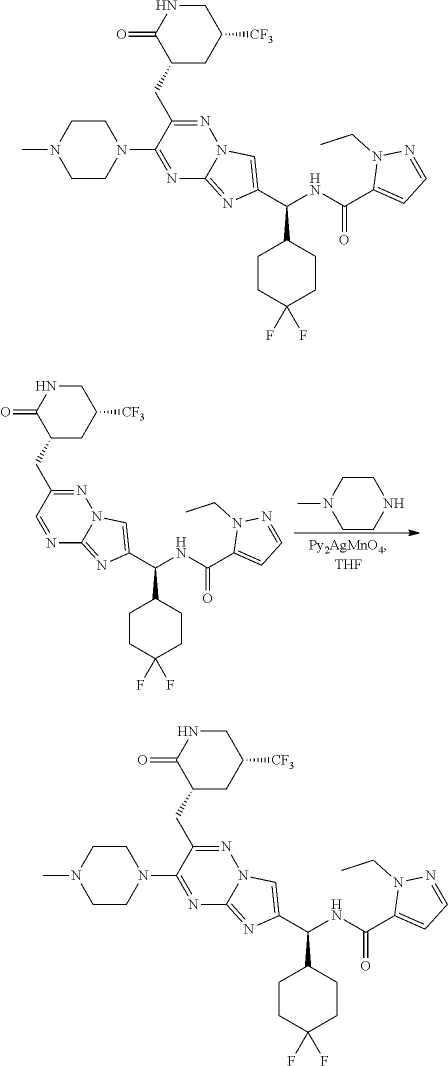

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with 1-methylpiperazine (176 mg, 1.76 mmol, 195 μL, 20.0 eq) was carried out following General Procedure 8, using bis(pyridine)silver(I) permanganate (170 mg, 439 μmol, 5.00 eq) in THF (0.09 M) and purifying by Prep-HPLC (column: Phenomenex luna C18 150*25 mm 10 um; mobile phase: [water (FA)—ACN]; gradient: 16%-36% B over 10 min) to afford N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (33.58 mg, 50.22 μmol, 57% yield).

### Example 245. Preparation of N—((S)-(4,4-difluorocyclohexyl)(3-((R)-3,4-dimethylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 298)

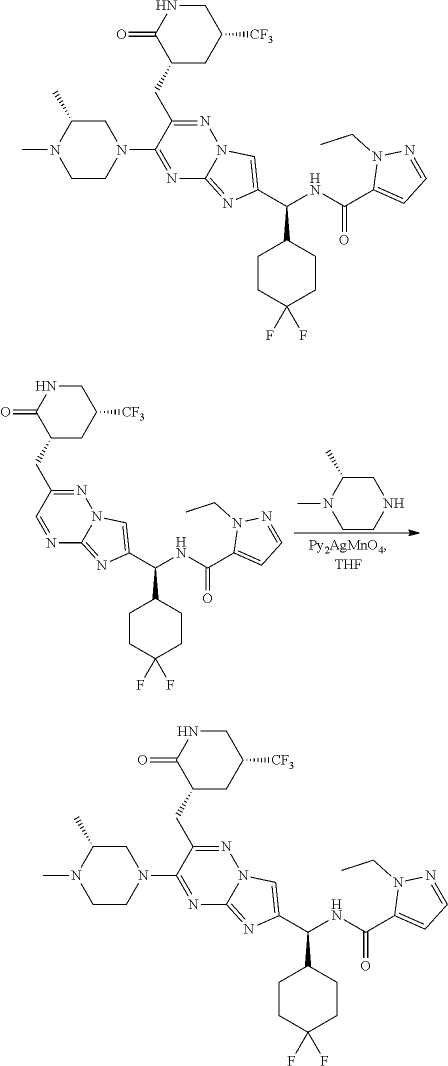

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (40.0 mg, 70.3 μmol, 1.00 eq) with (R)-1,2-dimethylpiperazine (160 mg, 1.41 mmol, 20.0 eq) was carried out following General Procedure 8, using bis(pyridine)silver(I) permanganate (40.8 mg, 105 μmol, 1.50 eq) in THF (0.04 M) and purifying by Prep-TLC (SiO₂, DCM/MeOH=10:1) then by Prep-HPLC (FA condition; column: Phenomenex luna C18 150*25 mm*10 um; mobile phase: [water (FA)—ACN]; gradient: 22%-42% B over 10 min) to afford N—((S)-(4,4-difluorocyclohexyl)(3-((R)-3,4-dimethylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (5.5 mg, 7.78 μmol, 11% yield) as a yellow solid.

### Example 246. Preparation of N—((S)-(4,4-difluorocyclohexyl)(3-(4-methyl-4,7-diazaspiro[2.5]octan-7-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 357)

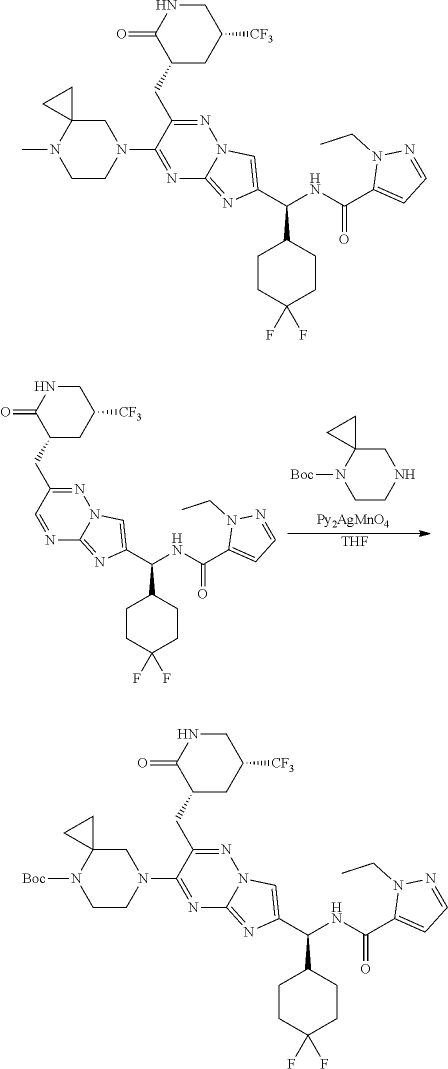 [SMILES: CCn1nccc1C(=O)N[C@H](c1cn2nc(C[C@H]3C[C@@H](C(F)(F)F)CNC3=O)c(N3CCN(C)C4(CC4)C3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCNCC12CC2 ; "THF" ; CCn1nccc1C(=O)N[C@H](c1cn2nc(C[C@H]3C[C@@H](C(F)(F)F)CNC3=O)c(N3CCN(C(=O)OC(C)(C)C)C4(CC4)C3)nc2n1)C1CCC(F)(F)CC1]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with tert-butyl 4,7-diazaspiro[2.5]octane-4-carboxylate (373 mg, 1.76 mmol, 20.0 eq) was carried out following General Procedure 8, using bis(pyridine)silver(I) permanganate (51.0 mg, 131 μmol, 1.50 eq) in THF (0.07 M) and purifying by Prep-TLC (SiO₂, DCM/MeOH=10:1) to afford tert-butyl 7-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-4,7-diazaspiro[2.5]octane-4-carboxylate (50.0 mg, 64.2 μmol, 73% yield) as a yellow solid. LCMS [M+H]⁺=779.4 m/z.

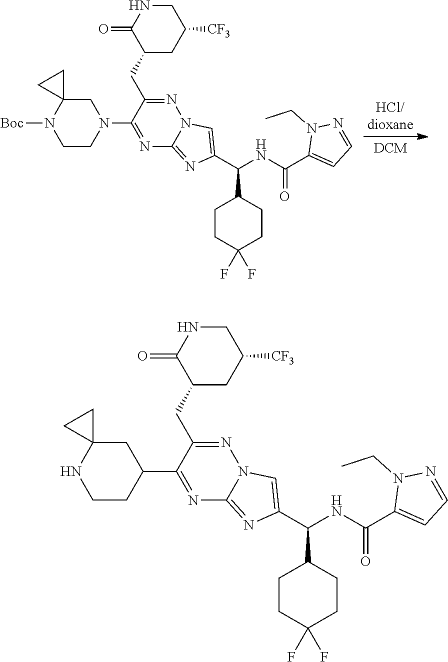 [SMILES: CCn1nccc1C(=O)N[C@H](c1cn2nc(C[C@H]3C[C@@H](C(F)(F)F)CNC3=O)c(N3CCN(C(=O)OC(C)(C)C)C4(CC4)C3)nc2n1)C1CCC(F)(F)CC1 ; CCn1nccc1C(=O)N[C@H](c1cn2nc(C[C@H]3C[C@@H](C(F)(F)F)CNC3=O)c(C3CCNC4(CC4)C3)nc2n1)C1CCC(F)(F)CC1]

To a solution of tert-butyl 7-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-4,7-diazaspiro[2.5]octane-4-carboxylate (40.0 mg, 51.3 μmol, 1.00 eq) in DCM (4.00 mL) was added HCl/dioxane (2.00 M, 128 μL, 5.00 eq) at 25° C. After stirring at room temperature for 1 h, the reaction mixture was concentrated under reduced pressure to remove HCl/dioxane and DCM and afford N—((S)-(4,4-difluorocyclohexyl)(2-(((3R,5R)-2-oxo-5-(trifluoromethyl)piperidin-3-yl)methyl)-3-(4,7-diazaspiro[2.5]octan-7-yl)imidazo[1,2-b][1,2,4]triazin-6-yl)methyl)-1-ethyl-1H-pyrazole-5-carboxamide (30.0 mg, crude HCl salt), obtained as a yellow solid. LCMS [M+H]⁺=679.4 m/z.

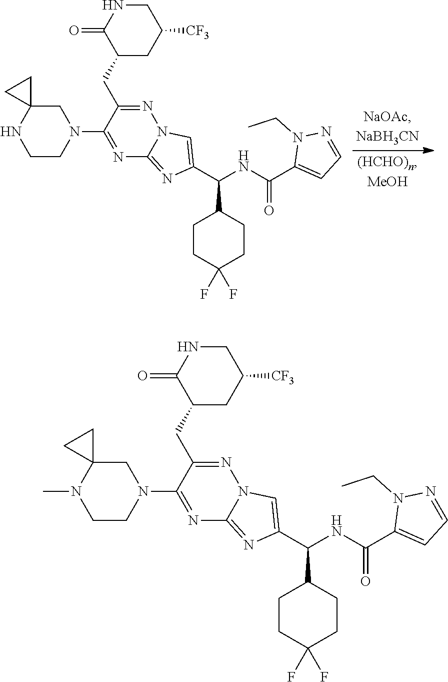 [SMILES: CCn1nccc1C(=O)N[C@H](c1cn2nc(C[C@H]3C[C@@H](C(F)(F)F)CNC3=O)c(N3CCNC4(CC4)C3)nc2n1)C1CCC(F)(F)CC1 ; CCn1nccc1C(=O)N[C@H](c1cn2nc(C[C@H]3C[C@@H](C(F)(F)F)CNC3=O)c(N3CCN(C)C4(CC4)C3)nc2n1)C1CCC(F)(F)CC1]

To a solution of N—((S)-(4,4-difluorocyclohexyl)(2-(((3R,5R)-2-oxo-5-(trifluoromethyl)piperidin-3-yl)methyl)-3-(4,7-diazaspiro[2.5]octan-7-yl)imidazo[1,2-b][1,2,4]triazin-6-yl)methyl)-1-ethyl-1H-pyrazole-5-carboxamide (30.0 mg, 1.00 eq, crude HCl salt) and paraformaldehyde (6.30 mg, 209 μmol, 5.78 μL, 5.00 eq) in MeOH (2.00 mL) was added NaOAc (13.7 mg, 167 μmol, 4.00 eq) and NaBH₃CN (5.27 mg, 83.9 μmol, 2.00 eq). The mixture was stirred at 25° C. for 1 h. The reaction mixture was then diluted with water and extracted with EtOAc (15.0 mL*3). The combined organic layers were dried over Na₂SO₄, filtered, and concentrated under reduced pressure to give a residue. The residue was purified by Prep-HPLC (FA condition; column: Phenomenex luna C18 150*25 mm*10 um; mobile phase: [water (FA)—ACN]; gradient: 22%-42% B over 10 min) to afford N—((S)-(4,4-difluorocyclohexyl)(3-(4-methyl-4,7-diazaspiro[2.5]octan-7-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (10.74 mg, 15.3 μmol, 30% yield over 2 steps) as a yellow solid.

### Example 247. Preparation of N—((S)-(4,4-difluorocyclohexyl)(3-((R)-3-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 235)

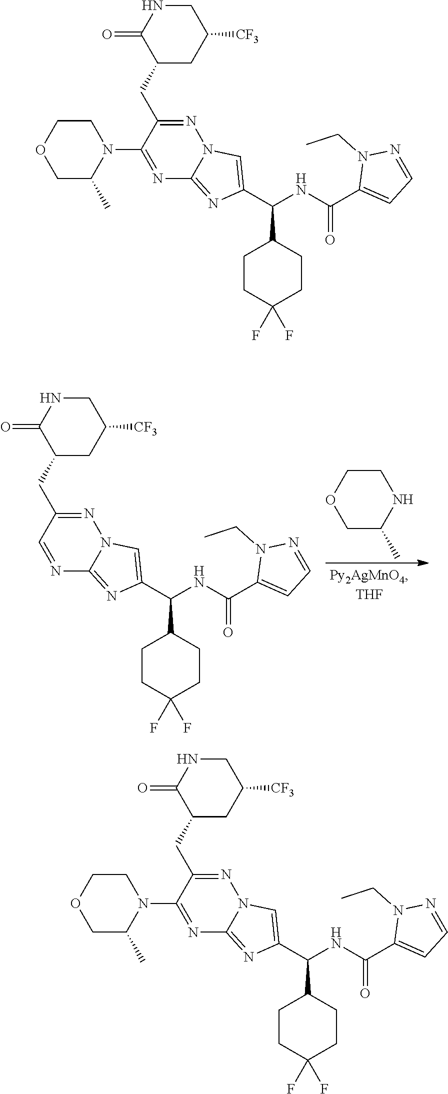

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.2 mg, 12.6 μmol, 1.00 eq) with (R)-3-methylmorpholine (280 mg, 2.77 mmol, 314.5 uL, 220 eq) was carried out following General Procedure 8, using bis(pyridine)silver(I) permanganate (110 mg, 285.7 μmol, 22.7 eq) in THF (0.025 M) and purifying by prep-HPLC (column: Gemini C18 250×21.2 mm, 5 μm; mobile phase: [water (TFA)—acetonitrile]; gradient: 10%-50% B over 30 min), then lyophilized directly to afford N—((S)-(4,4-difluorocyclohexyl)(3-((R)-3-methylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (0.47 mg, 0.6 μmol, 5% yield, TFA salt).

### Example 248. Preparation of N—((S)—((R)-3,3-difluorocyclohexyl)(3-((R)-2-methylmorpholino)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 305)

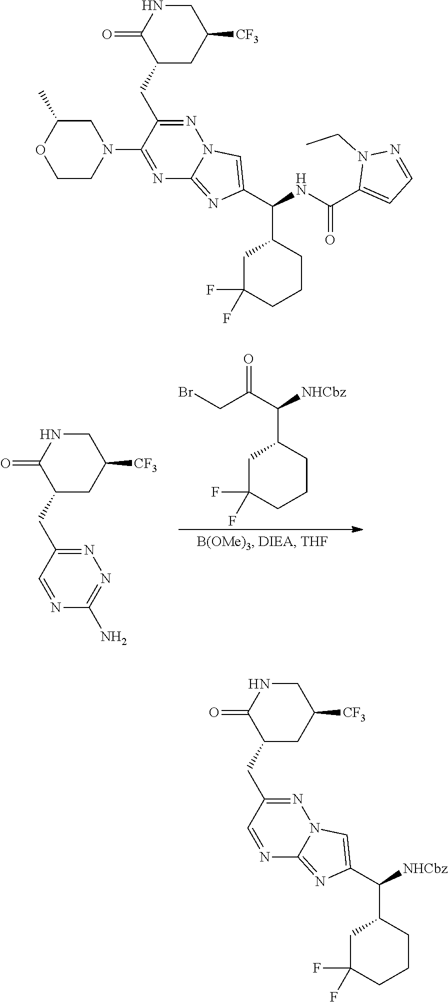

The synthesis of benzyl ((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate was carried out following General Procedure 3, using (3R,5S)-3-((3-amino-1,2,4-triazin-6-yl)methyl)-5-(trifluoromethyl)piperidin-2-one (500 mg, 1.82 mmol, 1.00 eq) and benzyl ((S)-3-bromo-1-((R)-3,3-difluorocyclohexyl)-2-oxopropyl)carbamate (881 mg, 2.18 mmol, 1.20 eq). The crude product was purified by column chromatography (SiO₂, DCM/MeOH=50/1 to 40/1) to afford benzyl ((S)—((R)-3,3-difluorocyclohexyl)(2-(((3R,5 S)-2-oxo-5-(trifluoromethyl)piperidin-3-yl)methyl)imidazo[1,2-b][1,2,4]triazin-6-yl)methyl)carbamate as a yellow solid (800 mg, 1.38 mmol, 76% yield). LCMS [M+H]⁺=581.4 m/z.

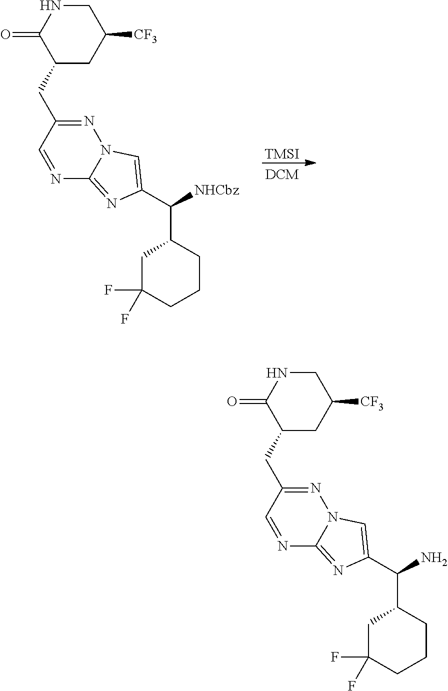 [SMILES: "TMSI" ; O=C(N[C@H](c1cn2nc(C[C@H]3C[C@H](C(F)(F)F)CNC3=O)cnc2n1)[C@@H]1CCCC(F)(F)C1)OCc1ccccc1 ; N[C@H](c1cn2nc(C[C@H]3C[C@H](C(F)(F)F)CNC3=O)cnc2n1)[C@@H]1CCCC(F)(F)C1]

The synthesis of (3R,5S)-3-((6-((S)-amino((R)-3,3-difluorocyclohexyl)methyl)imidazo[1,2-b][1,2,4]triazin-2-yl)methyl)-5-(trifluoromethyl)piperidin-2-one was carried out following General Procedure 4, using benzyl ((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)carbamate (800 mg, 1.38 mmol, 1.00 eq) and TMSI (1.10 g, 5.51 mmol, 750.28 μL, 4.00 eq) in DCM (0.14 M). Upon acid/base workup, (3R,5S)-3-((6-((S)-amino((R)-3,3-difluorocyclohexyl)methyl)imidazo[1,2-b][1,2,4]triazin-2-yl)methyl)-5-(trifluoromethyl)piperidin-2-one was obtained as a yellow solid (480 mg, 1.08 mmol, 78% yield). LCMS [M+H]⁺=447.2 m/z.

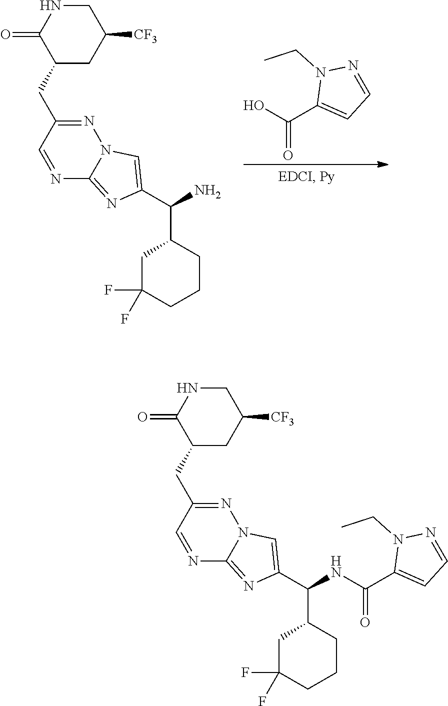

Amide coupling of (3R,5S)-3-((6-((S)-amino((R)-3,3-difluorocyclohexyl)methyl)imidazo[1,2-b][1,2,4]triazin-2-yl)methyl)-5-(trifluoromethyl)piperidin-2-one (200 mg, 448 μmol, 1.00 eq) with 1-ethyl-1H-pyrazole-5-carboxylic acid (81.6 mg, 582 μmol, 1.30 eq) using EDCI (257 mg, 1.34 mmol, 3.00 eq) in pyridine (0.22 M) was carried out following the conditions in General Procedure 6. The product was purified by prep-TLC (SiO₂, DCM/MeOH=10.1) to afford N—((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (208 mg, 365.85 μmol, 82% yield) as a light-yellow solid. LCMS [M+H]⁺=569.4 m/z.

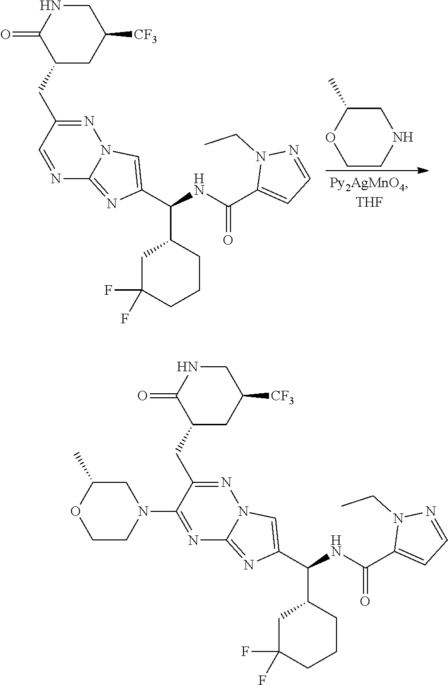

Oxidative nucleophilic substitution of hydrogen of N—((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.94 μmol, 1.00 eq) with (R)-2-methylmorpholine (88.9 mg, 879 μmol, 10.0 eq) was carried out following General Procedure 8, using bis(pyridine)silver(I) permanganate (5.00 eq) in THF (0.04 M) and purifying by prep-TLC (SiO₂, DCM/MeOH=10:1) to afford N—((S)—((R)-3,3-difluorocyclohexyl)(3-((R)-2-methylmorpholino)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (40.0 mg, 59.01 μmol, 67.10% yield) as a yellow solid.

### Example 249. Preparation of N—((S)—((R)-3,3-difluorocyclohexyl)(3-((S)-2-methylmorpholino)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 306)

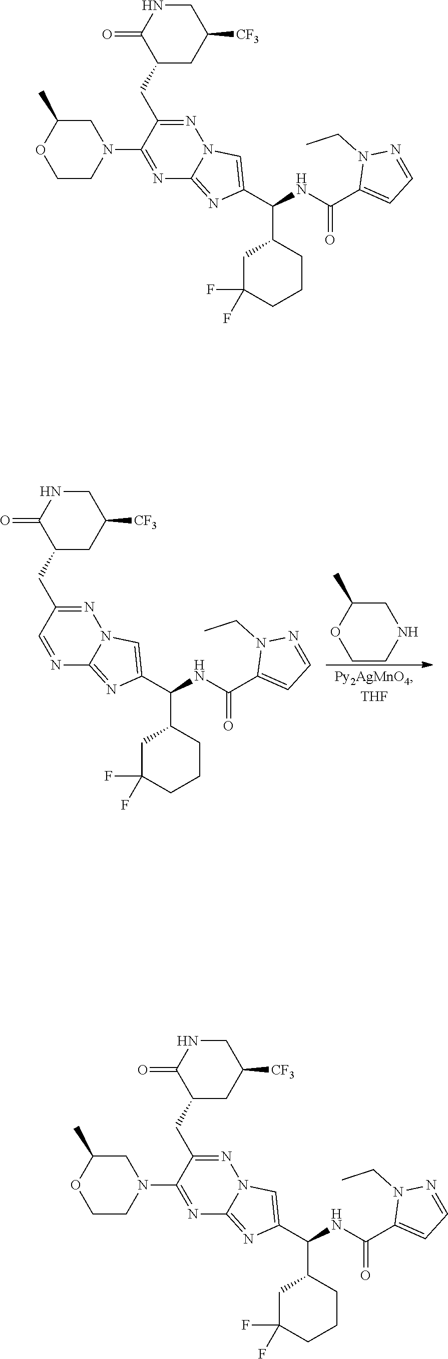

Oxidative nucleophilic substitution of hydrogen of N—((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with (S)-2-methylmorpholine (177 mg, 1.76 mmol, 20.0 eq) was carried out following General Procedure 8, using bis(pyridine)silver(I) permanganate (170 mg, 439 μmol, 5.00 eq) in THF (0.18 M) and purifying by prep-TLC (SiO₂, DCM/MeOH=10:1) to afford N—((S)—((R)-3,3-difluorocyclohexyl)(3-((S)-2-methylmorpholino)-2-(((3R,5S)-2-oxo-5-(trifluoromethyl)piperidin-3-yl)methyl)imidazo[1,2-b][1,2,4]triazin-6-yl)methyl)-1-ethyl-1H-pyrazole-5-carboxamide (31.27 mg, 45.5 μmol, 52% yield) as a yellow solid.

### Example 250. Preparation of N—((S)—((R)-3,3-difluorocyclohexyl)(3-((R)-2-methylmorpholino)-2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 309)

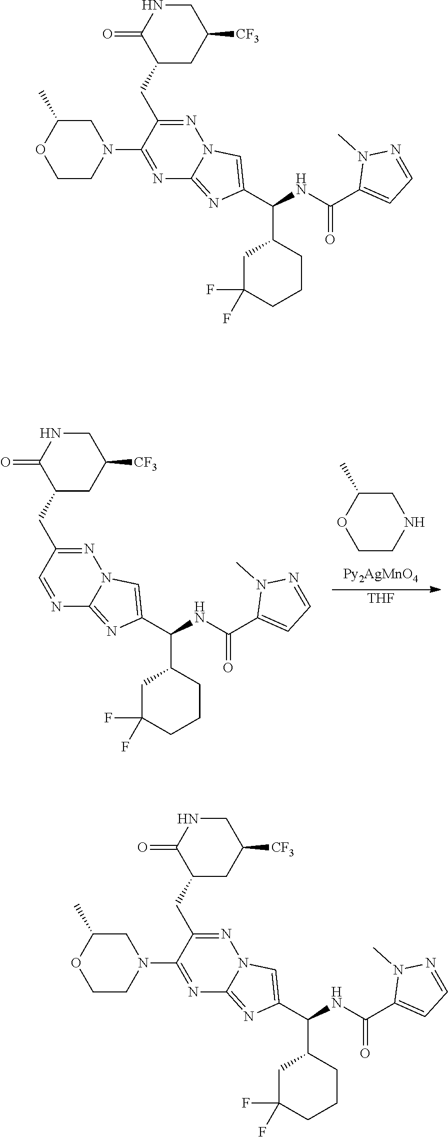

Oxidative nucleophilic substitution of hydrogen of N—((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (50.0 mg, 90.2 μmol, 1.00 eq) with (R)-2-methylmorpholine (91.2 mg, 901 μmol, 10.0 eq) was carried out following General Procedure 8, using bis(pyridine)silver(I) permanganate (40.0 mg, 450 μmol, 5.00 eq) in THF (0.05 M) and purifying by prep-TLC (SiO₂, DCM/MeOH=10:1) to afford N—((S)—((R)-3,3-difluorocyclohexyl)(3-((R)-2-methylmorpholino)-2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (40.0 g, 60.5 μmol, 67% yield) as a yellow solid.

### Example 251. Preparation of N—((S)—((R)-3,3-difluorocyclohexyl)(3-((S)-2-methylmorpholino)-2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 310)

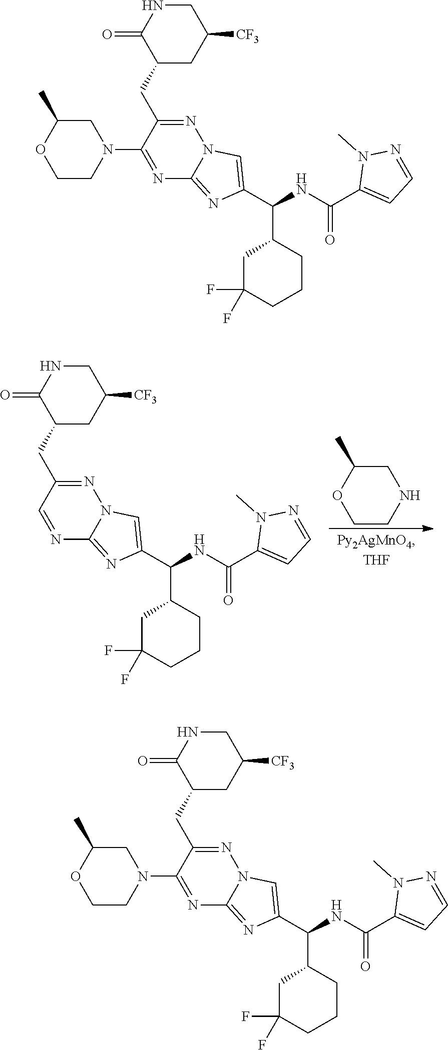

Oxidative nucleophilic substitution of hydrogen of N—((S)—((R)-3,3-difluorocyclohexyl)(2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (50.0 mg, 90.1 μmol, 1.00 eq) with (S)-2-methylmorpholine (182 mg, 1.80 mmol, 20.0 eq) was carried out following General Procedure 8, using bis(pyridine)silver(I) permanganate (174 mg, 450 μmol, 5.00 eq) in THF (0.18 M) and purifying by prep-TLC (SiO₂, DCM/MeOH=10:1) to afford N—((S)—((R)-3,3-difluorocyclohexyl)(3-((S)-2-methylmorpholino)-2-(((3R,5S)-2-oxo-5-(trifluoromethyl)piperidin-3-yl)methyl)imidazo[1,2-b][1,2,4]triazin-6-yl)methyl)-1-methyl-1H-pyrazole-5-carboxamide (30.7 mg, 46.0 μmol, 51% yield) as a yellow solid.

### Example 252. Preparation of N—((S)—((R)-3,3-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 307)

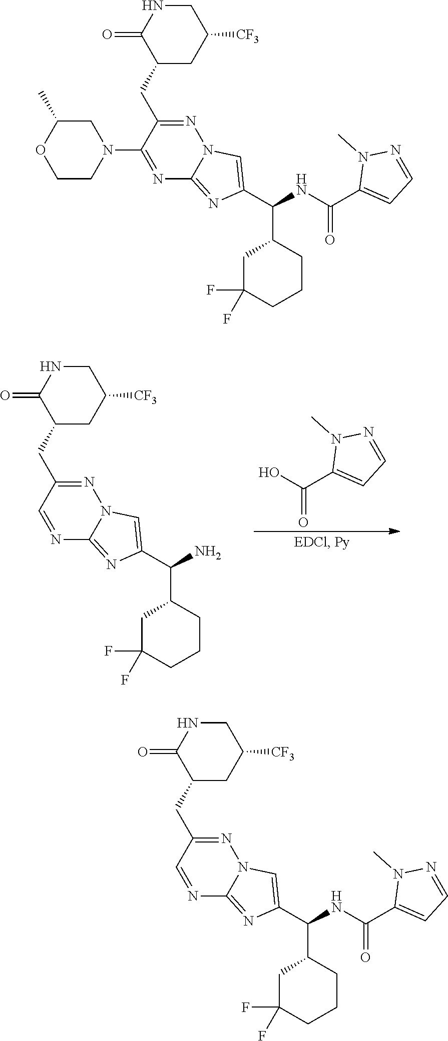

Amide coupling of intermediate (3R,5R)-3-((6-((S)-amino((R)-3,3-difluorocyclohexyl)methyl)imidazo[1,2-b][1,2,4]triazin-2-yl)methyl)-5-(trifluoromethyl)piperidin-2-one (180 mg, 403 μmol, 1.00 eq) with 1-methyl-1H-pyrazole-5-carboxylic acid (66.1 mg, 524 μmol, 1.30 eq) using EDCI (231 mg, 1.21 mmol, 3.00 eq) in pyridine (0.13 M) was carried out following the conditions in General Procedure 6. The product, N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (220 mg, 396.7 μmol, 98% yield), was obtained as a white solid. LCMS [M+H]⁺=555.3 m/z.

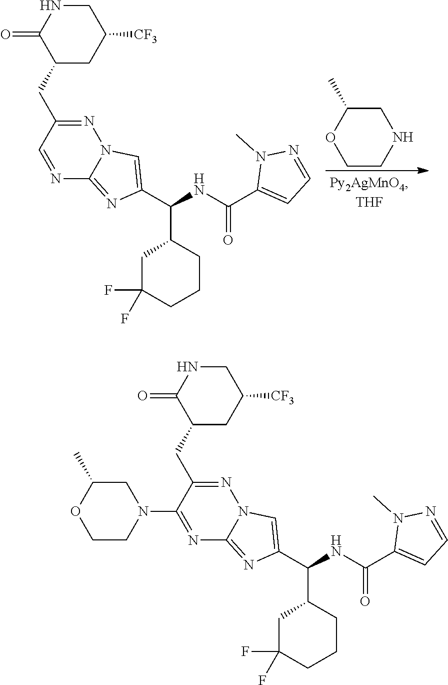

Oxidative nucleophilic substitution of hydrogen of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (0.06 g, 108 μmol, 1.00 eq) with (R)-2-methylmorpholine (218 mg, 2.16 mmol, 20.0 eq) was carried out following General Procedure 8, using bis(pyridine)silver(I) permanganate (209 mg, 541 μmol, 5.00 eq) in THF (0.11 M) and purifying by Prep-HPLC (column: Phenomenex luna C18 150*25 mm*10 um; mobile phase: [water (FA)—ACN]; gradient: 32%-52% B over 10 min) to afford N—((S)—((R)-3,3-difluorocyclohexyl)(3-((R)-2-methylmorpholino)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (33.9 mg, 51.4 μmol, 48% yield) as a yellow solid.

### Example 253. Preparation of N—((S)—((R)-3,3-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (Compound 308)

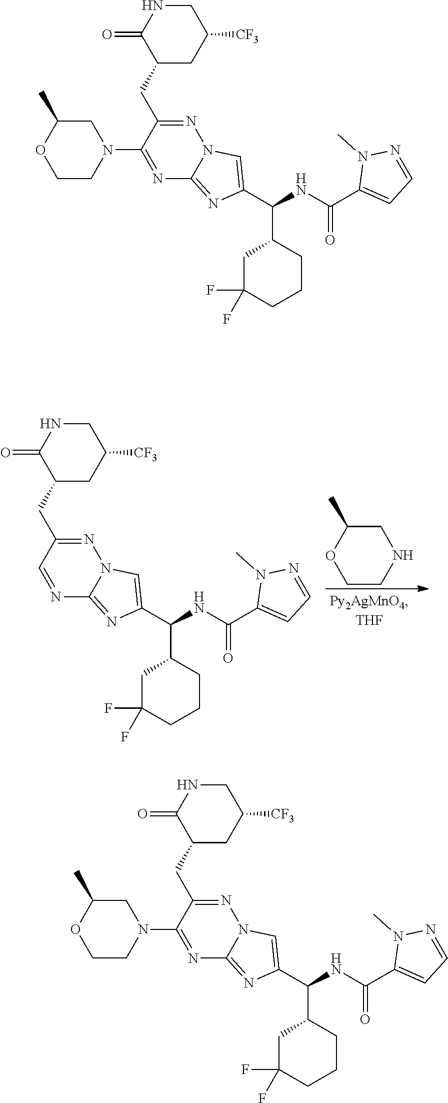

Oxidative nucleophilic substitution of hydrogen of N—((S)—((R)-3,3-difluorocyclohexyl)(2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (0.06 g, 108 μmol, 1.00 eq) with (S)-2-methylmorpholine (218 mg, 2.16 mmol, 20.0 eq) was carried out following General Procedure 8, using bis(pyridine)silver(I) permanganate (209 mg, 541 μmol, 5.00 eq) in THF (0.1 M) and purifying by Prep-HPLC (column: Phenomenex luna C18 150*25 mm*10 um; mobile phase: [water (FA)—ACN]; gradient: 32%-52% B over 10 min) to afford N—((S)—((R)-3,3-difluorocyclohexyl)(3-((S)-2-methylmorpholino)-2-(((3R,5R)-2-oxo-5-(trifluoromethyl)piperidin-3-yl)methyl)imidazo[1,2-b][1,2,4]triazin-6-yl)methyl)-1-methyl-1H-pyrazole-5-carboxamide (33.59 mg, 51.3 μmol, 47% yield) as a yellow solid.

### Example 254. Preparation of N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (Compound 311)

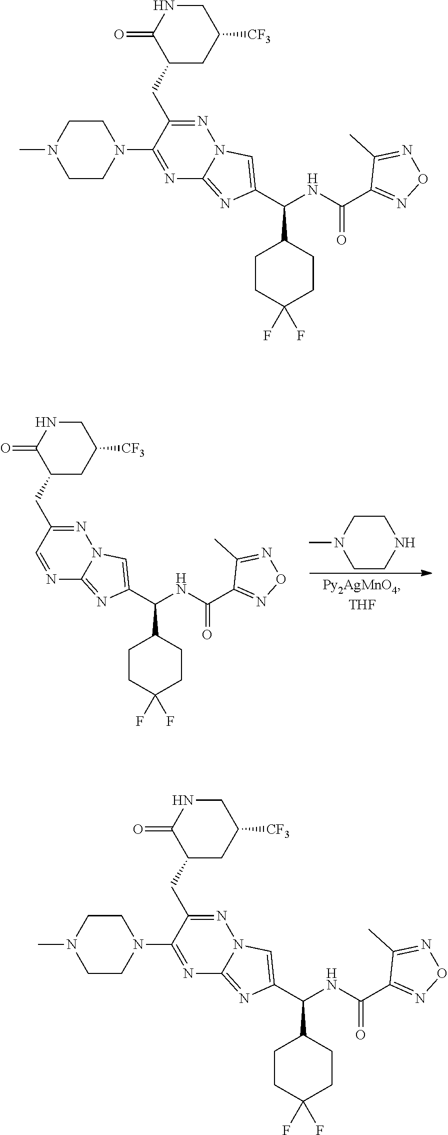

Oxidative nucleophilic substitution of hydrogen of intermediate N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (50.0 mg, 89.8 μmol, 1.00 eq) with 1-methylpiperazine (179 mg, 1.80 mmol, 199 μL, 20.0 eq) was carried out following General Procedure 8, using bis(pyridine)silver(I) permanganate (173 mg, 449 μmol, 5.00 eq) in THF (0.18 M) and purifying by prep-TLC (SiO₂, DCM/MeOH=10:1) then Prep-SFC (condition: column: DAICEL CHIRALCEL OD (250 mm*30 mm, 10 um); mobile phase: [CO₂-i-PrOH]; B %: 50%, isocratic elution mode) to afford N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (22.77 mg, 34.8 μmol, 39% yield) as a yellow solid.

### Example 255. Preparation of N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (Compound 312)

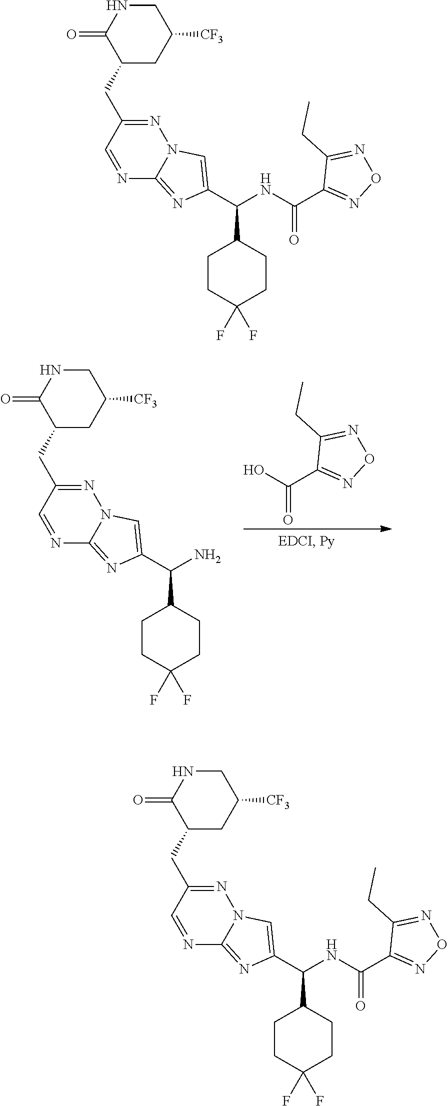

Amide coupling of intermediate (3R,5R)-3-((6-((S)-amino(4,4-difluorocyclohexyl)methyl)imidazo[1,2-b][1,2,4]triazin-2-yl)methyl)-5-(trifluoromethyl)piperidin-2-one (100 mg, 224 μmol, 1.00 eq) with 4-ethyl-1,2,5-oxadiazole-3-carboxylic acid (63.6 mg, 448 μmol, 2.00 eq) using EDCI (128 mg, 672 μmol, 3.00 eq) in pyridine (0.2 M) was carried out following the conditions in General Procedure 6. The product was purified by prep-TLC (SiO₂, DCM/MeOH=10:1) then by Prep-HPLC (condition: column: Phenomenex luna C18 150*25 mm*10 um; mobile phase: [water (FA)—ACN]; gradient: 45%-75% B over 10 min) to afford N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (60.0 mg, 105 μmol, 47% yield) as a yellow solid. LCMS [M+H]⁺=571.3 m/z.

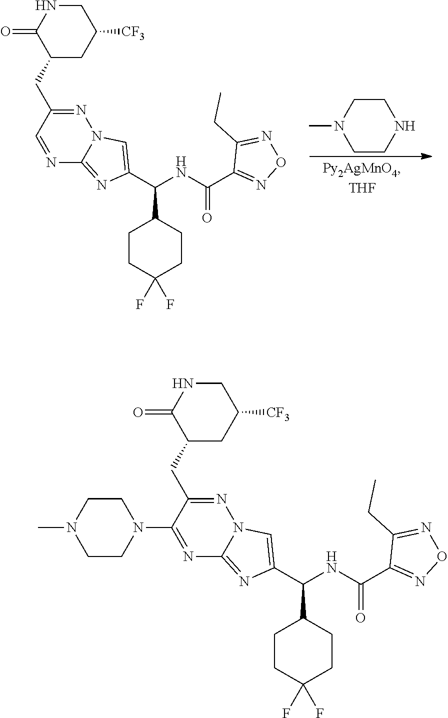

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (50.0 mg, 87.6 μmol, 1.00 eq) with 1-methylpiperazine (175 mg, 1.75 mmol, 194 μL, 20.0 eq) was carried out following General Procedure 8, using bis(pyridine)silver(I) permanganate (169 mg, 438 μmol, 5.00 eq) in THF (0.13 M) and purifying by prep-TLC (SiO₂, DCM/MeOH=10:1) then by SFC (condition: column: DAICEL CHIRALCEL OD (250 mm*30 mm, 10 um); mobile phase: [CO₂-i-PrOH]; B %: 50%, isocratic elution mode) to afford N—((S)-(4,4-difluorocyclohexyl)(3-(4-methylpiperazin-1-yl)-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (30.2 mg, 44.6 μmol, 51% yield) as a yellow solid.

### Example 256. Preparation of N—((S)-(4,4-difluorocyclohexyl)(3-((2-methoxyethyl)(methyl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 237)

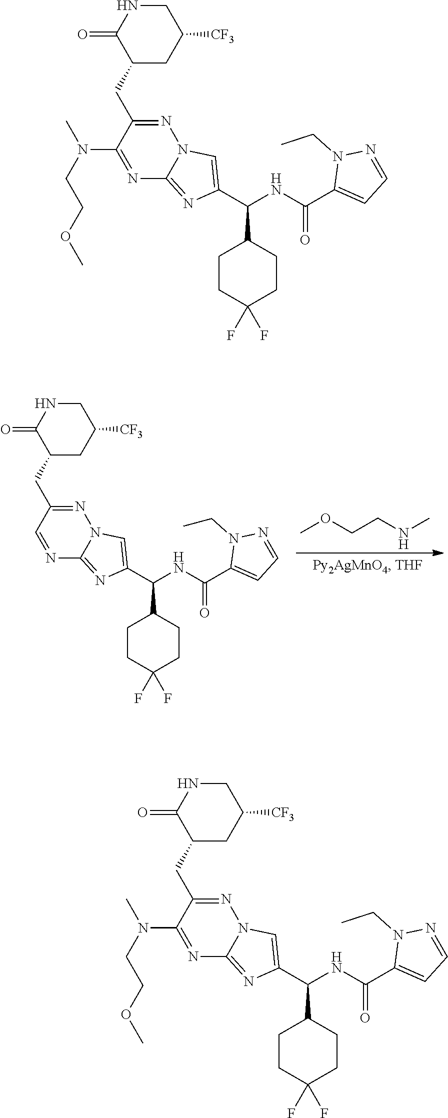

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.6 mg, 13.3 μmol, 1.00 eq) with 2-methoxy-N-methylethan-1-amine (23.7 mg, 265.9 μmol, 20.0 eq) was carried out following General Procedure 8, using bis(pyridine)silver(I) permanganate (10.5 mg, 27.3 μmol, 2.05 eq) in THF (0.05 M) and purifying by prep-HPLC (column: Gemini C18 250×21.2 mm, 5 μm; mobile phase: [water (TFA)—acetonitrile]; gradient: 10%-50% B over 30 min) followed by a basic workup to afford N—((S)-(4,4-difluorocyclohexyl)(3-((2-methoxyethyl)(methyl)amino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (5.2 mg, 7.9 μmol, 59% yield) as a yellow solid.

### Example 257. Preparation of N—((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 326)

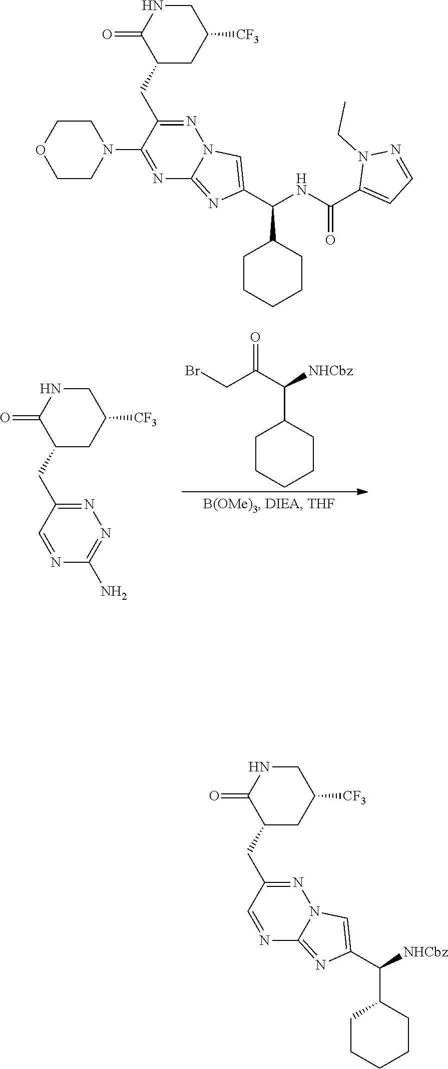

The synthesis of benzyl ((S)-cyclohexyl(2-(((3R,5R)-2-oxo-5-(trifluoromethyl)piperidin-3-yl)methyl)imidazo[1,2-b][1,2,4]triazin-6-yl)methyl)carbamate was carried out following General Procedure 3, using (3R,5R)-3-((3-amino-1,2,4-triazin-6-yl)methyl)-5-(trifluoromethyl)piperidin-2-one (200 mg, 726 μmol, 1.00 eq) and benzyl (S)-(3-bromo-1-cyclohexyl-2-oxopropyl)carbamate (500 mg, 1.36 mmol, 1.87 eq) and was purified by Prep-TLC (SiO₂ EtOAc) to afford benzyl ((S)-cyclohexyl(2-(((3R,5R)-2-oxo-5-(trifluoromethyl)piperidin-3-yl)methyl)imidazo[1,2-b][1,2,4]triazin-6-yl)methyl)carbamate (200 mg, 367.3 μmol, 51% yield) as a white solid. LCMS [M+H]⁺=545.3 m/z.

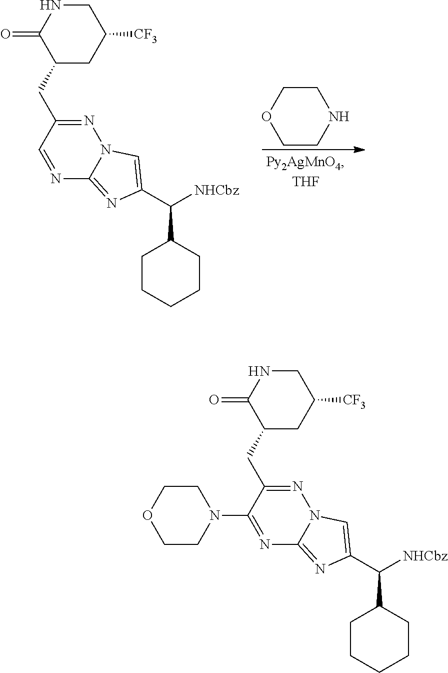 [SMILES: O=C(N[C@H](c1cn2nc(C[C@H]3C[C@@H](C(F)(F)F)CNC3=O)cnc2n1)C1CCCCC1)OCc1ccccc1 ; C1COCCN1 ; O=C(N[C@H](c1cn2nc(C[C@H]3C[C@@H](C(F)(F)F)CNC3=O)c(N3CCOCC3)nc2n1)C1CCCCC1)OCc1ccccc1]

Oxidative nucleophilic substitution of hydrogen of benzyl ((S)-cyclohexyl(2-(((3R,5R)-2-oxo-5-(trifluoromethyl)piperidin-3-yl)methyl)imidazo[1,2-b][1,2,4]triazin-6-yl)methyl)carbamate (140 mg, 257 μmol, 1.00 eq) with morpholine (447 mg, 5.14 mmol, 452 μL, 20.0 eq) was carried out following General Procedure 8, using bis(pyridine)silver(I) permanganate (497 mg, 1.29 mmol, 5.00 eq) in TUE (0.09 M) and purifying by Prep-TLC (SiO₂ EtOAc) to afford benzyl ((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)carbamate (100 mg crude) as a solid. LCMS [M+H]⁺=630.4 m/z.

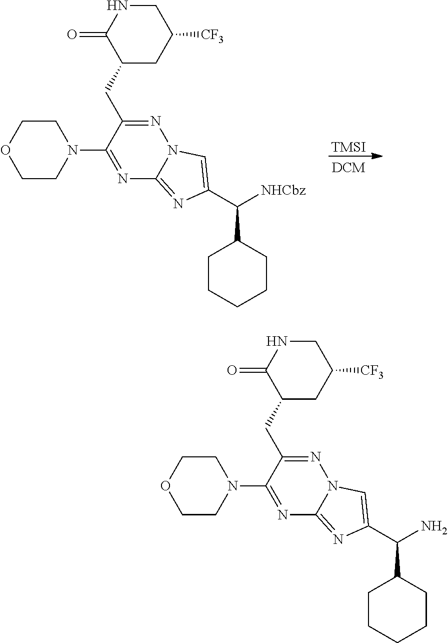 [SMILES: "TMSI" ; O=C(N[C@H](c1cn2nc(C[C@H]3C[C@@H](C(F)(F)F)CNC3=O)c(N3CCOCC3)nc2n1)C1CCCCC1)OCc1ccccc1 ; N[C@H](c1cn2nc(C[C@H]3C[C@@H](C(F)(F)F)CNC3=O)c(N3CCOCC3)nc2n1)C1CCCCC1]

The synthesis of (3R,5R)-3-((6-((S)-amino(cyclohexyl)methyl)-3-morpholinoimidazo[1,2-b][1,2,4]triazin-2-yl)methyl)-5-(trifluoromethyl)piperidin-2-one was carried out following General Procedure 4, using benzyl ((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)carbamate (0.09 g, 142 μmol, 1.00 eq) and TMSI (85.8 mg, 428 μmol, 58.3 μL, 3.00 eq) in DCM (0.05 M) to afford (3R,5R)-3-((6-((S)-amino(cyclohexyl)methyl)-3-morpholinoimidazo[1,2-b][1,2,4]triazin-2-yl)methyl)-5-(trifluoromethyl)piperidin-2-one (60 mg, 121.1 μmol, 77% yield over 2 steps) as a solid. LCMS [M+H]⁺=496.2 m/z.

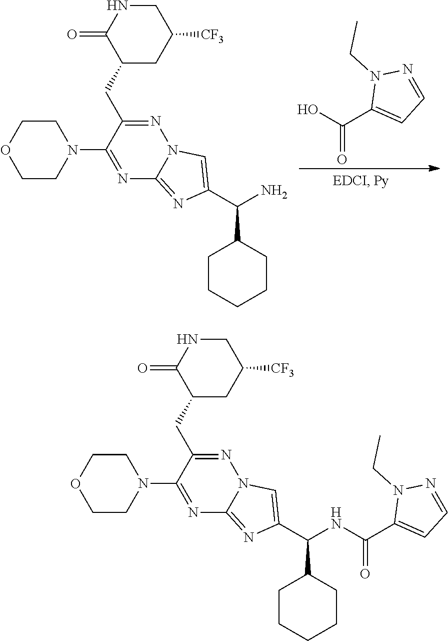

Amide coupling of (3R,5R)-3-((6-((S)-amino(cyclohexyl)methyl)-3-morpholinoimidazo[1,2-b][1,2,4]triazin-2-yl)methyl)-5-(trifluoromethyl)piperidin-2-one (30.0 mg, 60.5 μmol, 1.00 eq) with 1-ethyl-1H-pyrazole-5-carboxylic acid (11.0 mg, 78.7 μmol, 1.30 eq) using EDCI (34.8 mg, 181 μmol, 3.00 eq) in pyridine (0.03 M) was carried out following the conditions in General Procedure 6. The product was purified by Prep-HPLC (column: Phenomenex luna C18 150*25 mm*10 um; mobile phase: [water (FA)—ACN], gradient: 28%-48% B over 10 min) to afford N—((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (25.1 mg, 40.5 μmol, 67% yield) as a yellow solid.

### Example 258. Preparation of N—((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (Compound 327)

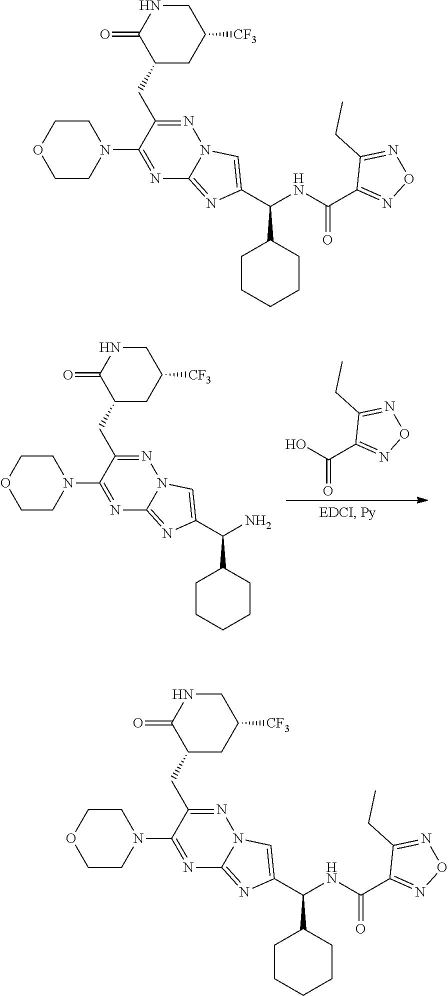

Amide coupling of (3R,5R)-3-((6-((S)-amino(cyclohexyl)methyl)-3-morpholinoimidazo[1,2-b][1,2,4]triazin-2-yl)methyl)-5-(trifluoromethyl)piperidin-2-one (30.0 mg, 60.5 μmol, 1.00 eq) with 4-ethyl-1,2,5-oxadiazole-3-carboxylic acid (11.1 mg, 78.7 μmol, 1.30 eq) using EDCI (34.8 mg, 181 μmol, 3.00 eq) in pyridine (0.03 M) was carried out following the conditions in General Procedure 6. Crude product was purified by Prep-HPLC (column: Phenomenex luna C18 150*25 mm*10 um; mobile phase: [water (FA)—ACN]; gradient: 37%-57% B over 10 min) to afford N—((S)-cyclohexyl(3-morpholino-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (25.9 mg, 41.8 μmol, 69% yield) as a solid.

### Example 259. Preparation of tert-butyl (S)-4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-2-methylpiperazine-1-carboxylate (Compound 238)

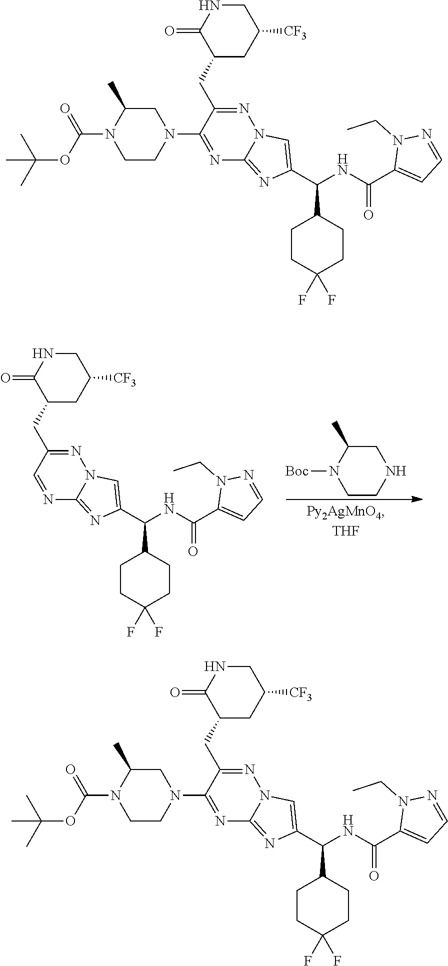 [SMILES: CCn1nccc1C(=O)N[C@H](c1cn2nc(C[C@H]3C[C@@H](C(F)(F)F)CNC3=O)c(N3CCN(C(=O)OC(C)(C)C)[C@@H](C)C3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; C[C@H]1CNCCN1C(=O)OC(C)(C)C ; CCn1nccc1C(=O)N[C@H](c1cn2nc(C[C@H]3C[C@@H](C(F)(F)F)CNC3=O)c(N3CCN(C(=O)OC(C)(C)C)[C@@H](C)C3)nc2n1)C1CCC(F)(F)CC1]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.3 mg, 12.6 μmol, 1.00 eq) with tert-butyl (S)-2-methylpiperazine-1-carboxylate (46.3 mg, 231.2 μmol, 18.4 eq) was carried out following General Procedure 8, using bis(pyridine)silver(I) permanganate (12.1 mg, 31.4 μmol, 2.5 eq) in THF (0.05 M) and purifying by column chromatography (SiO₂, DCM/MeOH=10/0 to 10/1) to afford tert-butyl (S)-4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-2-methylpiperazine-1-carboxylate (2.5 mg, 3.2 μmol, 25% yield) as a neon-yellow film.

### Example 260. Preparation of N—((S)-(4,4-difluorocyclohexyl)(3-((S)-3-methylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 239)

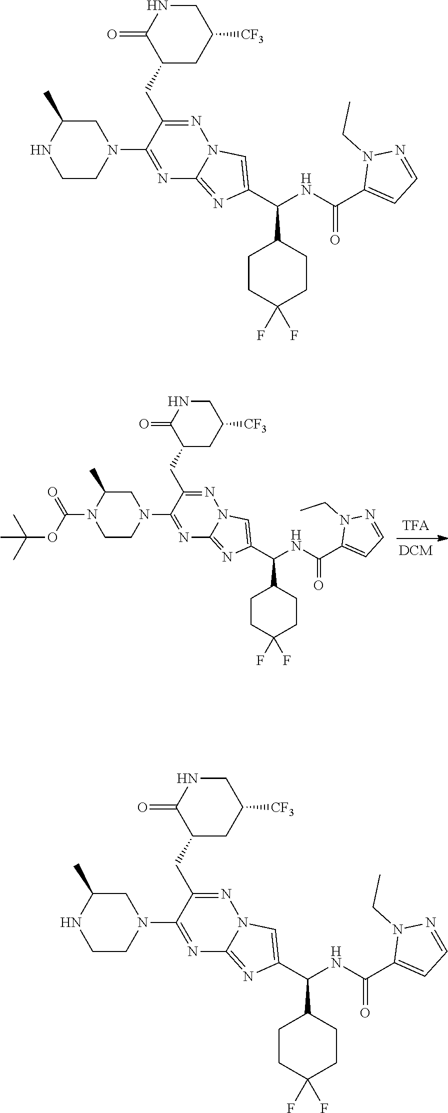

To a solution of tert-butyl (S)-4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-2-methylpiperazine-1-carboxylate (26.0 mg, 32.7 μmol, 1.00 eq) in DCM (0.065 M) was added TFA (0.25 mL, 3.27 mmol, 100 eq) at 25° C. After stirring for 2 hours at room temperature, the reaction mixture was concentrated to remove all volatiles. Upon lyophilization, N—((S)-(4,4-difluorocyclohexyl)(3-((S)-3-methylpiperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (6.8 mg, 7.7 μmol, 24% yield, TFA salt) was obtained as an orange solid.

### Example 261. Preparation of N-((1S)-(4,4-difluorocyclohexyl)(3-(6-methyl-3,6-diazabicyclo[3.1.1]heptan-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 360)

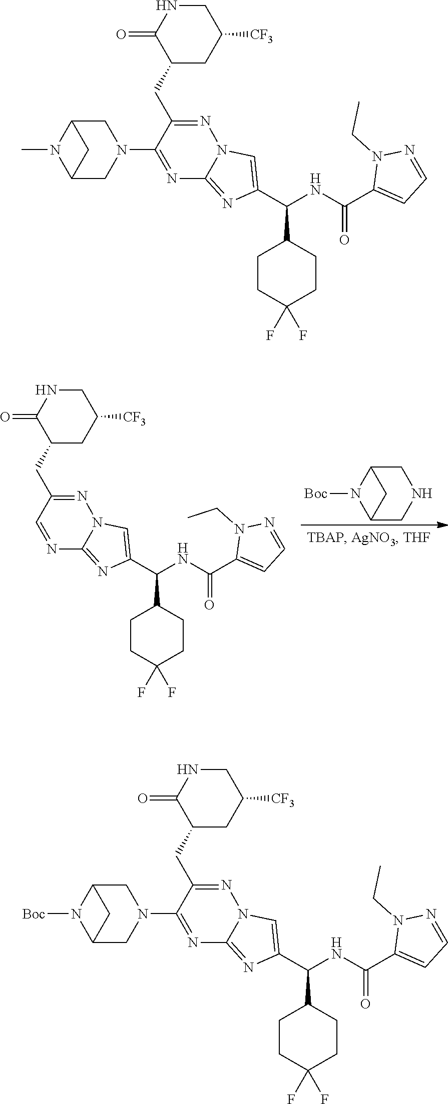

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (200 mg, 351 μmol, 1.00 eq) with tert-butyl 3,6-diazabicyclo[3.1.1]heptane-6-carboxylate (906 mg, 4.57 mmol, 13.0 eq) was carried out following General Procedure 14, using AgNO₃ (119 mg, 703 μmol, 2.00 eq) and TBAP (254 mg, 703 μmol, 2.00 eq) in THF (0.07 M), stirring for 20 min, then purifying by Prep-TLC (SiO₂ EtOAc) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-3,6-diazabicyclo[3.1.1]heptane-6-carboxylate (150 mg, 196.1 μmol, 56% yield) as a solid. LCMS [M+H]⁺=765.4 m/z.

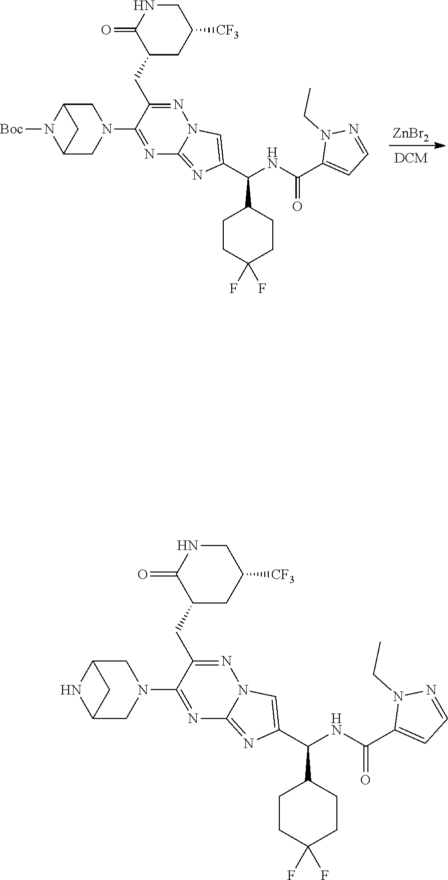

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-3,6-diazabicyclo[3.1.1]heptane-6-carboxylate (60.0 mg, 78.4 μmol, 1.00 eq) in DCM (3.00 mL) was added ZnBr₂ (176 mg, 784 μmol, 39.2 μL, 10.0 eq). The mixture was stirred at 25° C. for 2 h. The reaction mixture was concentrated under reduced pressure to give crude N-((1 S)-(3-(3,6-diazabicyclo[3.1.1]heptan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, crude) as a solid. LCMS [M+H]⁺=665.4 m/z.

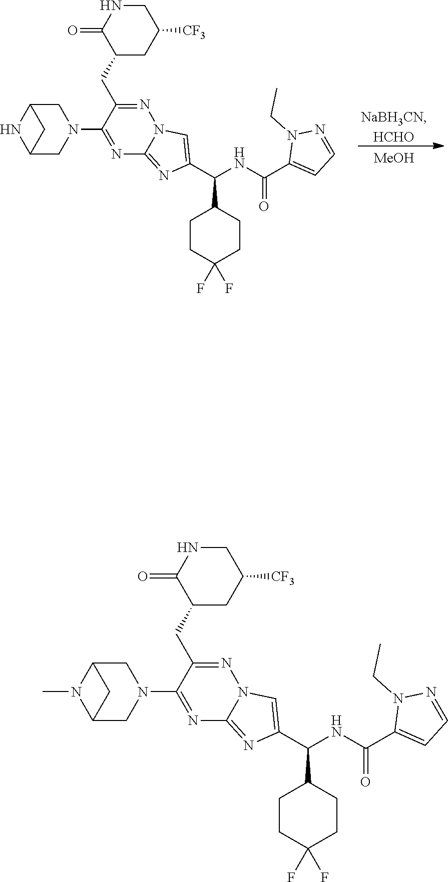

To a solution of crude N-((1S)-(3-(3,6-diazabicyclo[3.1.1]heptan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg) in MeOH (5.00 mL) was added HCHO (30.5 mg, 376 μmol, 28.0 μL, 37.0% purity, 5.00 eq) and NaBH₃CN (18.9 mg, 300 mol, 4.00 eq). The mixture was stirred at 25° C. for 2 h. The reaction mixture was then filtered and concentrated under reduced pressure to give a residue. The crude product was purified by Prep-HPLC (column: Waters Xbridge 150*25 mm*5 um; mobile phase: [water (NH₄HCO₃)—ACN]; gradient: 27%-47% B over 10 min). The product, N-((1 S)-(4,4-difluorocyclohexyl)(3-(6-methyl-3,6-diazabicyclo[3.1.1]heptan-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (35.0 mg, 49.9 μmol, 66% yield), was obtained as a white solid.

### Example 262. Preparation of N-((1S)-(4,4-difluorocyclohexyl)(3-(8-methyl-3,8-diazabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 361)

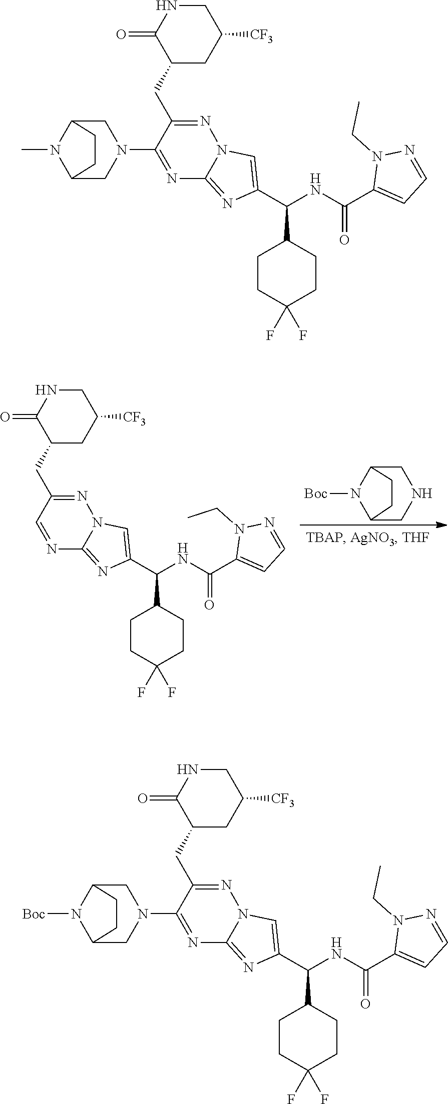

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (100 mg, 175 μmol, 1.00 eq) with tert-butyl 3,8-diazabicyclo[3.2.1]octane-8-carboxylate (485 mg, 2.29 mmol, 13.0 eq) was carried out following General Procedure 14, using AgNO₃ (60.0 mg, 353 μmol, 2.01 eq) and TBAP (190 mg, 527 μmol, 3.00 eq) in THF (0.04 M), stirring for 30 min at 0° C., then purifying by prep-TLC (SiO₂, EtOAc) to afford tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-3,8-diazabicyclo[3.2.1]octane-8-carboxylate (90.0 mg, 115 μmol, 66% yield) as a yellow solid. LCMS [M+H]⁺=779.4 m/z.

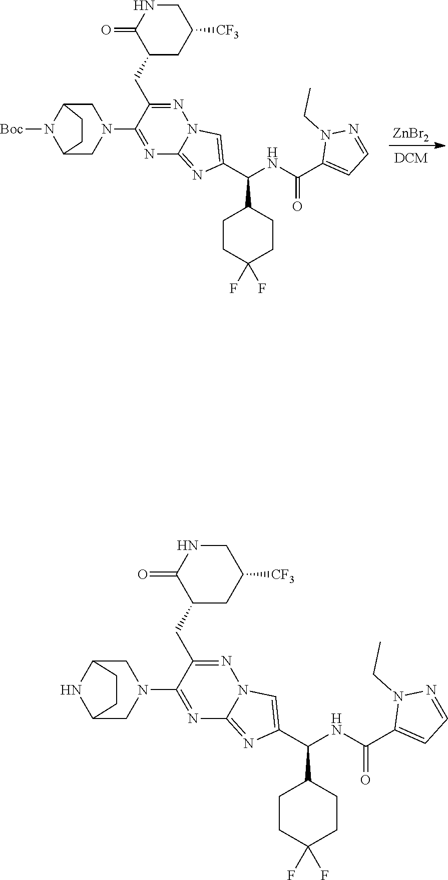

To a solution of tert-butyl 3-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)-3,8-diazabicyclo[3.2.1]octane-8-carboxylate (70.0 mg, 89.8 μmol, 1.00 eq) in DCM (2.00 mL) was added ZnBr₂ (161 mg, 719 μmol, 35.9 μL, 8.00 eq). The mixture was stirred at 25° C. for 6 h. The reaction mixture was concentrated under reduced pressure to give crude N-((1S)-(3-(3,8-diazabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (60.0 mg) as a yellow solid which was used directly in the next step without further purification. LCMS [M+H]⁺=679.3 m/z.

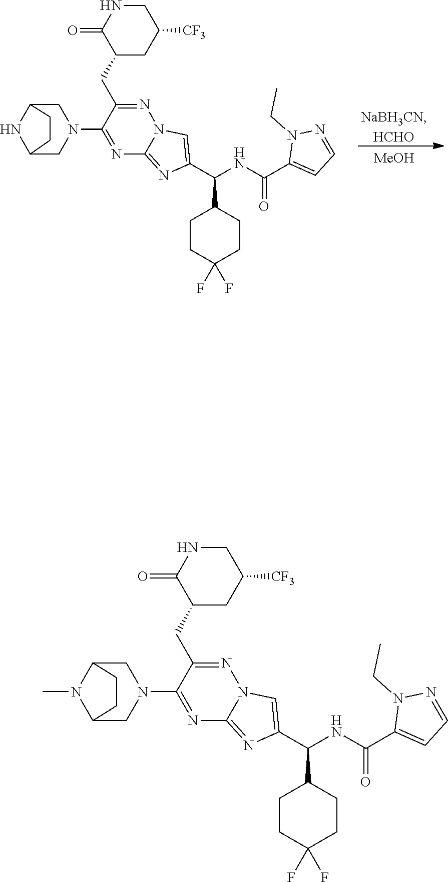

To a solution of crude N-((1S)-(3-(3,8-diazabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (60.0 mg, crude) in MeOH (1.00 mL) was added formaldehyde (932 mg, 11.4 mmol, 855 μL, 37% purity, 130 eq) and NaBH₃CN (16.6 mg, 265 μmol, 3.00 eq). The mixture was stirred at 25° C. for 0.5 h. The reaction mixture was concentrated under reduced pressure to give a residue which was purified by prep-HPLC (column: Waters xbridge 150*25 mm 10 um; mobile phase: [water (NH₄HCO₃)—ACN]; gradient: 38%-58% B over 8 min) to afford N-((1S)-(4,4-difluorocyclohexyl)(3-(8-methyl-3,8-diazabicyclo[3.2.1]octan-3-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (24.05 mg, 33.43 μmol, 37% yield over 2 steps) as a yellow solid.

### Example 263. Preparation of N—((S)-(4,4-difluorocyclohexyl)(3-(2,2-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 365)

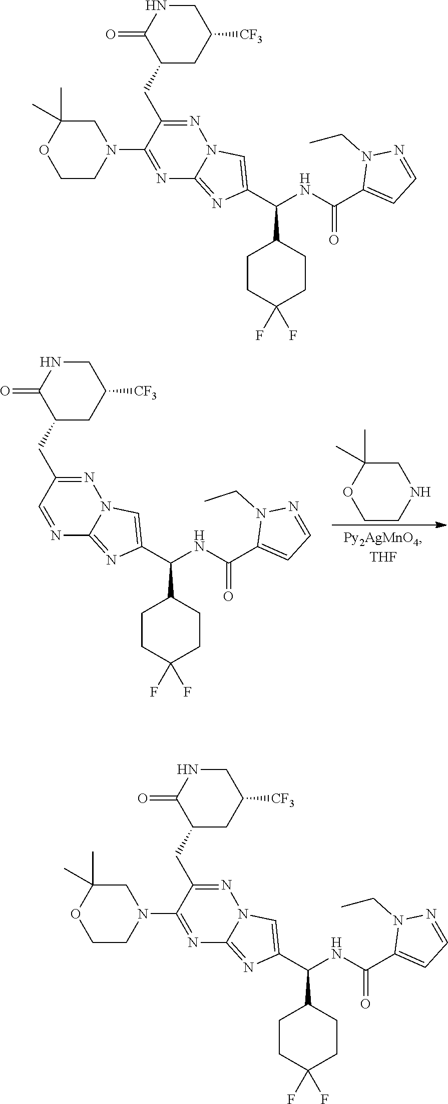

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (6.5 mg, 11.2 μmol, 1.00 eq) with 2,2-dimethylmorpholine (25.8 mg, 224 μmol, 20.0 eq) was carried out following General Procedure 8, using bis(pyridine)silver(I) permanganate (8.1 mg, 21.0 μmol, 1.9 eq) in THF (0.05 M) and purifying by prep-HPLC (column: Gemini C18 250×21.2 mm, 5 um; mobile phase: [water (TFA)—acetonitrile]; gradient: 10%-55% B over 25 min) followed by a basic workup to afford N—((S)-(4,4-difluorocyclohexyl)(3-(2,2-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (2.57 mg, 3.7 μmol, 33% yield) as a yellow solid.

### Example 264. Preparation of N—((S)-(4,4-difluorocyclohexyl)(3-((S)-hexahydropyrazino[2,1-c][1,4]oxazin-8(1H)-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 366)

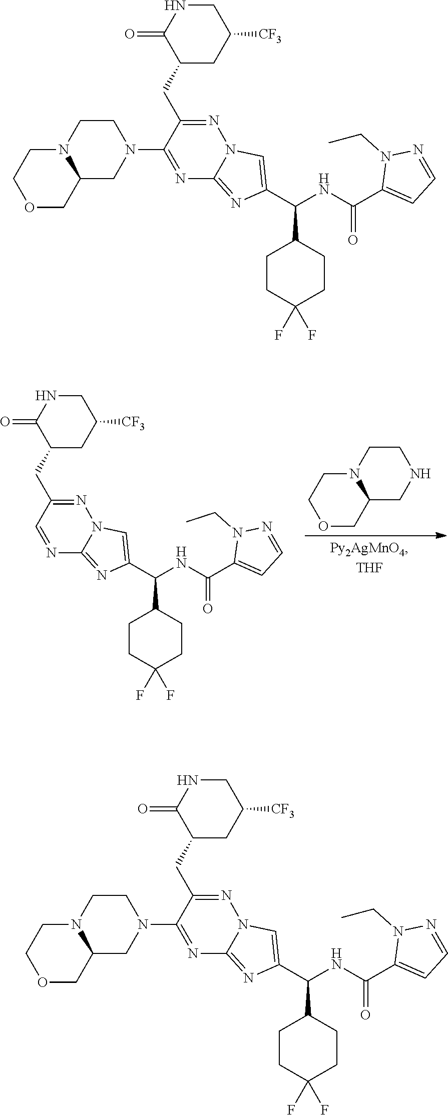

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with (S)-octahydropyrazino[2,1-c][1,4]oxazine (250 mg, 1.76 mmol, 20.0 eq) was carried out following General Procedure 8, using bis(pyridine)silver(I) permanganate (170 mg, 439 μmol, 5.00 eq) in THF (0.02 M) and purifying by Prep-TLC (SiO₂, EtOAc/MeOH=20:1) then Prep-HPLC (FA condition, column: Phenomenex luna C18 150*25 mm*10 um; mobile phase: [water (FA)—ACN]; gradient: 20%-40% B over 10 min) to afford N—((S)-(4,4-difluorocyclohexyl)(3-((S)-hexahydropyrazino[2,1-c][1,4]oxazin-8(1H)-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (12.14 mg, 17.1 μmol, 19% yield) as a yellow solid.

### Example 265. Preparation of N—((S)-(4,4-difluorocyclohexyl)(3-((R)-octahydro-2H-pyrido[1,2-a]pyrazin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 367)

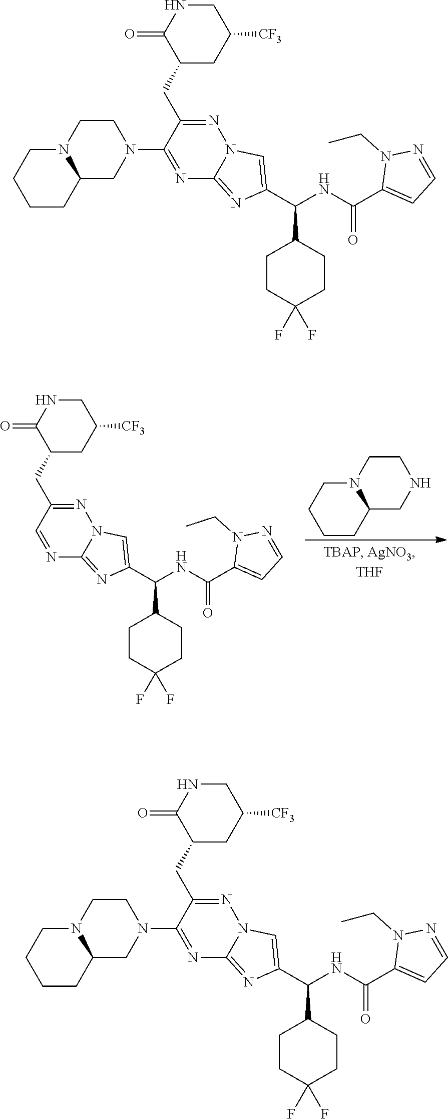

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (35.0 mg, 61.5 μmol, 1.00 eq) with (R)-octahydro-2H-pyrido[1,2-a]pyrazine (103 mg, 738 μmol, 12.0 eq) was carried out following General Procedure 14, using AgNO₃ (0.120 g, 706 μmol, 11.4 eq) and TBAP (66.7 mg, 184 μmol, 3.00 eq) in THF (0.06 M), stirring for 30 min at 0° C., then purifying by prep-TLC (SiO₂, EtOAc) and by Prep-HPLC (column: Waters Xbridge 150*25 mm*5 um; mobile phase: [water (ammonia hydroxide v/v)—ACN]; gradient: 39%-69% B over 9 min) to afford N—((S)-(4,4-difluorocyclohexyl)(3-((R)-octahydro-2H-pyrido[1,2-a]pyrazin-2-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (9.49 mg, 13.4 μmol, 22% yield) as a yellow solid.

### Example 266. Preparation of N—((S)-(4,4-difluorocyclohexyl)(3-((R)-hexahydropyrrolo[1,2-a]pyrazin-2(1H)-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 368)

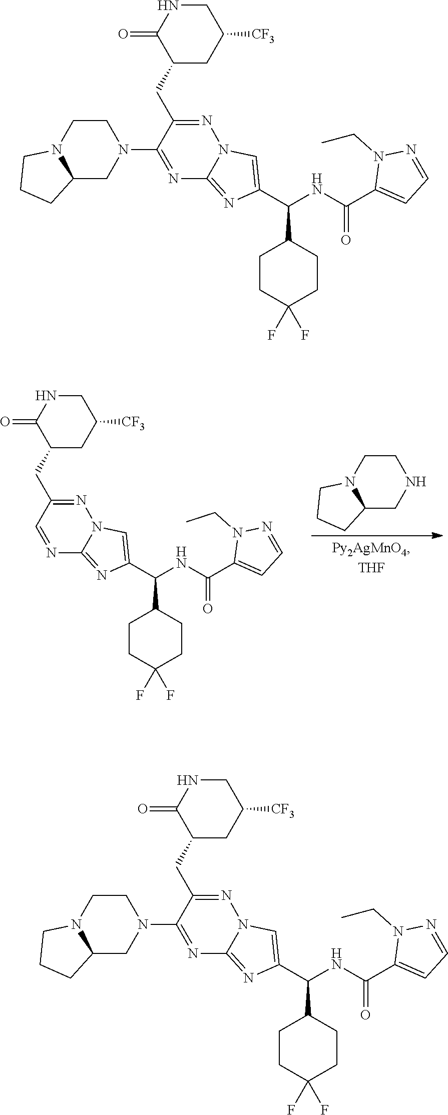

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (45.0 mg, 79.1 μmol, 1.00 eq) with (R)-octahydropyrrolo[1,2-a]pyrazine (99.8 mg, 791 μmol, 10.0 eq) was carried out following General Procedure 8, using bis(pyridine)silver(I) permanganate (153 mg, 395 μmol, 5 eq) in THF (0.08 M) and purifying by prep-TLC (SiO₂, EtOAc/MeOH=5:1) to afford N—((S)-(4,4-difluorocyclohexyl)(3-((R)-hexahydropyrrolo[1,2-a]pyrazin-2(1H)-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (5.38 mg, 6.52 μmol, 8.2% yield, 84% purity) as a yellow solid.

### Example 267. Preparation of N—((S)-(4,4-difluorocyclohexyl)(3-((2R,6R)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 241)

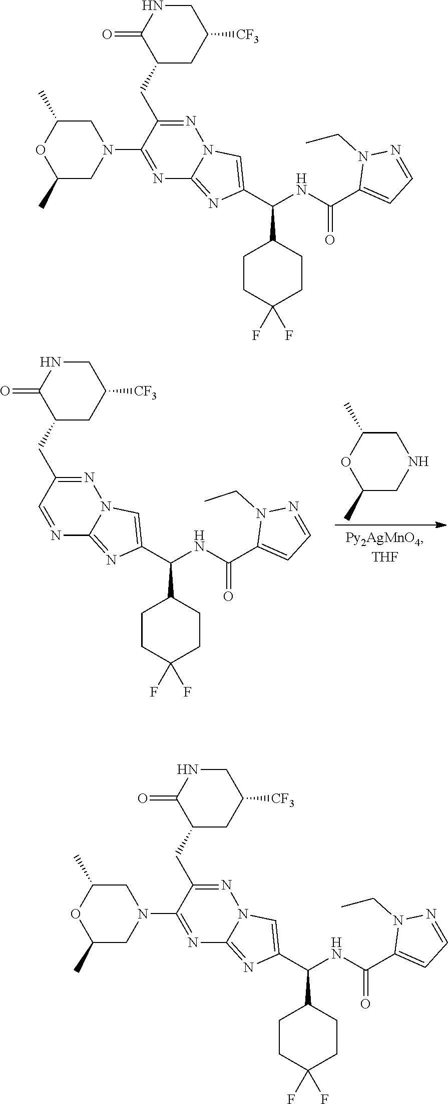

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (6.3 mg, 10.9 μmol, 1.00 eq) with (2R,6R)-2,6-dimethylmorpholine (12.5 mg, 108.6 μmol, 10.0 eq) was carried out following General Procedure 8, using bis(pyridine)silver(I) permanganate (17.8 mg, 46.2 μmol, 4.3 eq) in THF (0.05 M) and purifying by prep-HPLC (column: Gemini C18 250×21.2 mm, 5 μm; mobile phase: [water (TFA)—acetonitrile]; gradient: 10%-55% B over 27 min) followed by a basic workup to afford N—((S)-(4,4-difluorocyclohexyl)(3-((2R,6R)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (3.1 mg, 4.5 μmol, 41% yield) as a yellow solid.

### Example 268. Preparation of N—((S)-(4,4-difluorocyclohexyl)(3-(4-(2-methoxyethyl)piperazin-1-yl)-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (Compound 421)

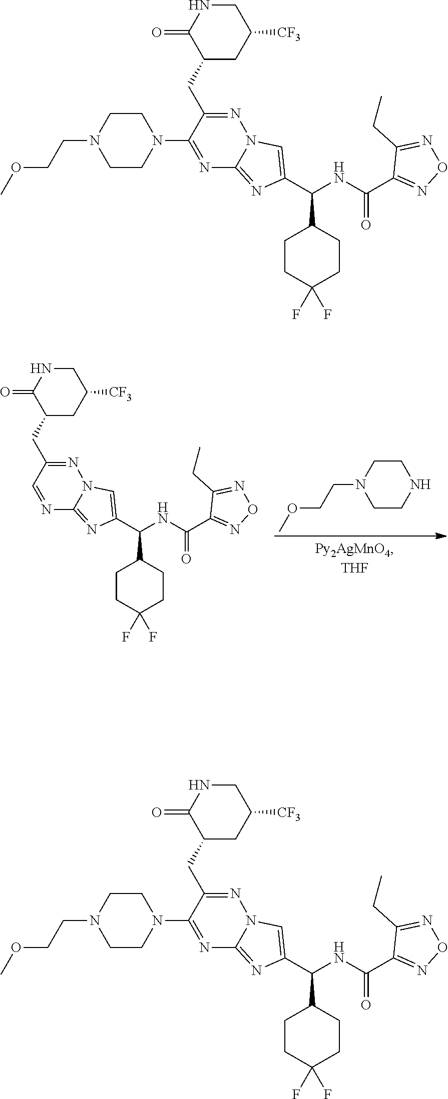

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (100 mg, 175 μmol, 1.00 eq) with 1-(2-methoxyethyl)piperazine (505 mg, 3.51 mmol, 521 μL, 20.0 eq) was carried out following General Procedure 8, using bis(pyridine)silver(I) permanganate (67.8 mg, 175 μmol, 1.00 eq) in THF (0.18 M) and purifying by Prep-HPLC (FA condition: column: Phenomenex luna C 18 150*25 mm*10 um; mobile phase: [water (FA)—ACN]; gradient: 26%-50% B over 8 min) to afford N—((S)-(4,4-difluorocyclohexyl)(3-(4-(2-methoxyethyl)piperazin-1-yl)-2-(((3R,5R)-2-oxo-5-(trifluoromethyl)piperidin-3-yl)methyl)imidazo[1,2-b][1,2,4]triazin-6-yl)methyl)-4-ethyl-1,2,5-oxadiazole-3-carboxamide (42.50 mg, 57.9 μmol, 33% yield) as a yellow solid.

### Example 269. Preparation of N—((S)-(3-(4-(tert-butyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 242)

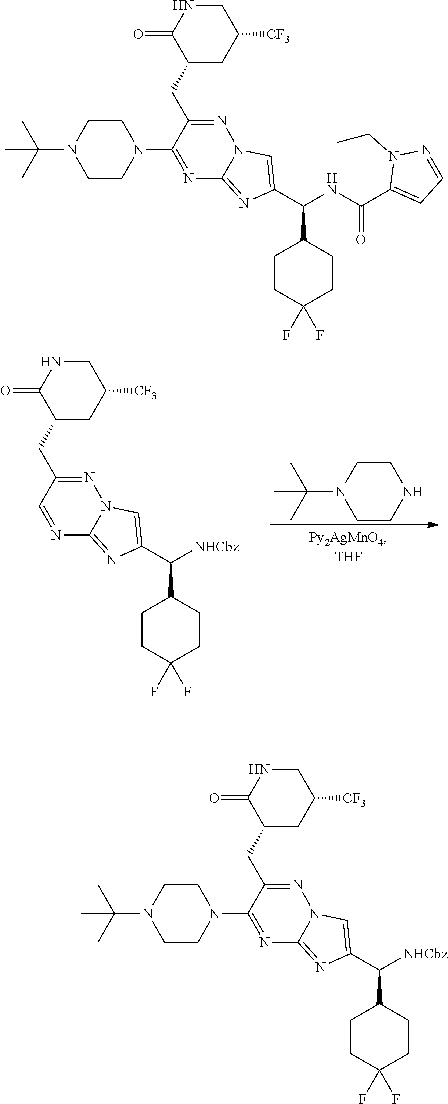

Oxidative nucleophilic substitution of hydrogen of benzyl ((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)carbamate (20.8 mg, 35.8 μmol, 1.00 eq) with 1-(tert-butyl)piperazine (55.2 mg, 388.1 umol, 10.8 eq) was carried out following General Procedure 8, using bis(pyridine)silver(I) permanganate (186.1 mg, 483.4 μmol, 13.5 eq) in THF (0.05 M) and purifying by column chromatography (SiO₂, DCM/MeOH=1:0 to 5:1) to afford crude benzyl ((S)-(3-(4-(tert-butyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)carbamate as a yellow oil (11.4 mg, crude), which was used directly in the next step without further purification. LCMS [M+H]⁺=721.3 m/z.

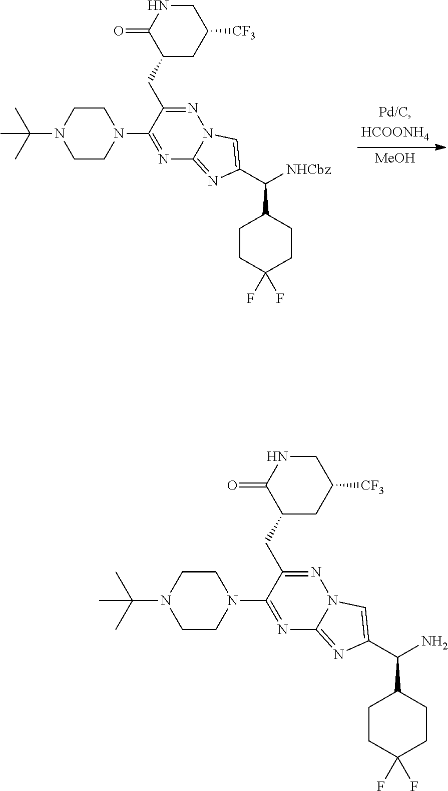

To a solution of crude benzyl ((S)-(3-(4-(tert-butyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)carbamate (11.4 mg, crude) and HCOONH₄ (52.7 mg) in MeOH (0.02 M) was added Pd/C (21.3 mg, 10.0% purity). The reaction mixture was stirred at 60° C. for 1.5 h. The reaction mixture was then filtered through Celite and concentrated to give crude (3R,5R)-3-((6-((S)-amino(4,4-difluorocyclohexyl)methyl)-3-(4-(tert-butyl)piperazin-1-yl)imidazo[1,2-b][1,2,4]triazin-2-yl)methyl)-5-(trifluoromethyl)piperidin-2-one (8.0 mg) as a yellow solid, which was used directly in the next step without further purification. LCMS [M+H]⁺=587.3 m/z.

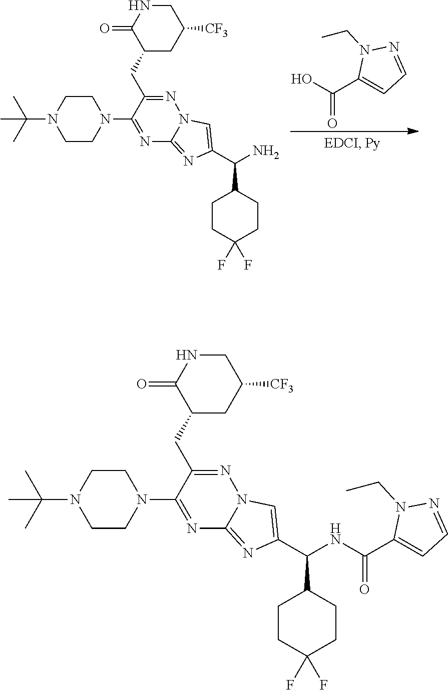

Amide coupling of crude (3R,5R)-3-((6-((S)-amino(4,4-difluorocyclohexyl)methyl-3-(4-(tert-butyl)piperazin-1-yl)imidazo[1,2-b][1,2,4]triazin-2-yl)methyl)-5-(trifluoromethyl)piperidin-2-one (8.0 mg) with 1-ethyl-1H-pyrazole-5-carboxylic acid (3.6 mg, 25.7 umol) using EDCI (4.0 mg, 20.9 umol) in 1:1 DCM/pyridine (0.03 M) was carried out following the conditions in General Procedure 6. The product was purified by prep-HPLC (column: Gemini C18 250×21.2 mm, 5 μm; mobile phase: [water (TFA)—acetonitrile]; gradient: 10%-40% B over 30 min) followed by a basic workup to afford N—((S)-(3-(4-(tert-butyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (3.2 mg, 4.3 μmol, 12% yield over 3 steps) as a yellow solid.

### Example 270. Preparation of N—((S)-(4,4-difluorocyclohexyl)(3-(4-methyl-4,7-diazaspiro[2.5]octan-7-yl)-2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (Compound 411)

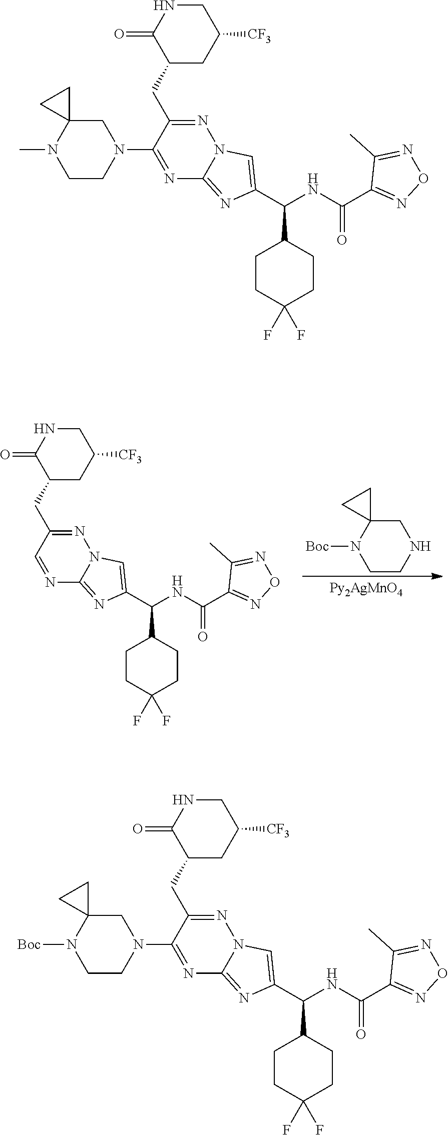 [SMILES: Cc1nonc1C(=O)N[C@H](c1cn2nc(C[C@H]3C[C@@H](C(F)(F)F)CNC3=O)c(N3CCN(C)C4(CC4)C3)nc2n1)C1CCC(F)(F)CC1 ; Cc1nonc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCNCC12CC2 ; Cc1nonc1C(=O)N[C@H](c1cn2nc(C[C@H]3C[C@@H](C(F)(F)F)CNC3=O)c(N3CCN(C(=O)OC(C)(C)C)C4(CC4)C3)nc2n1)C1CCC(F)(F)CC1]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (90.0 mg, 161 μmol, 1.00 eq) with tert-butyl 4,7-diazaspiro[2.5]octane-4-carboxylate (686 mg, 3.23 mmol, 20.0 eq) was carried out following General Procedure 8, using bis(pyridine)silver(I) permanganate (187 mg, 485 μmol, 3.00 eq) in THF (0.1 M) and purifying by Prep-TLC (SiO₂, DCM/MeOH=10:1) to afford tert-butyl 7-(6-((S)-(4,4-difluorocyclohexyl)(4-methyl-1,2,5-oxadiazole-3-carboxamido)methyl)-2-(((3R,5R)-2-oxo-5-(trifluoromethyl)piperidin-3-yl)methyl)imidazo[1,2-b][1,2,4]triazin-3-yl)-4,7-diazaspiro[2.5]octane-4-carboxylate (80.0 mg, 104 μmol, 65% yield) as a yellow solid. LCMS [M+H]⁺=767.4 m/z.

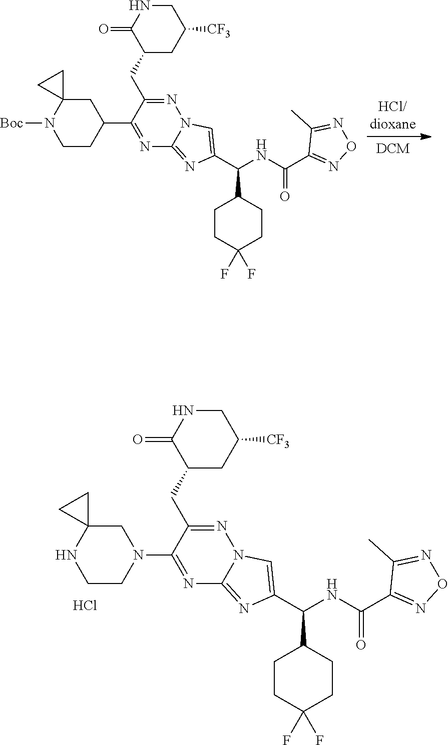 [SMILES: Cc1nonc1C(=O)N[C@H](c1cn2nc(C[C@H]3C[C@@H](C(F)(F)F)CNC3=O)c(C3CCN(C(=O)OC(C)(C)C)C4(CC4)C3)nc2n1)C1CCC(F)(F)CC1 ; Cc1nonc1C(=O)N[C@H](c1cn2nc(C[C@H]3C[C@@H](C(F)(F)F)CNC3=O)c(N3CCNC4(CC4)C3)nc2n1)C1CCC(F)(F)CC1 ; Cl]

To a solution of tert-butyl 7-(6-((S)-(4,4-difluorocyclohexyl)(4-methyl-1,2,5-oxadiazole-3-carboxamido)methyl)-2-(((3R,5R)-2-oxo-5-(trifluoromethyl)piperidin-3-yl)methyl)imidazo[1,2-b][1,2,4]triazin-3-yl)-4,7-diazaspiro[2.5]octane-4-carboxylate (80.0 mg, 104 μmol, 1.00 eq) in DCM (1.00 mL) was added HCl/dioxane (2.00 M, 0.500 mL, 9.58 eq). The mixture was stirred at 0° C. for 1 h. The reaction mixture was concentrated under reduced pressure to remove HCl/dioxane and DCM to give N—((S)-(4,4-difluorocyclohexyl)(2-(((3R,5R)-2-oxo-5-(trifluoromethyl)piperidin-3-yl)methyl)-3-(4,7-diazaspiro[2.5]octan-7-yl)imidazo[1,2-b][1,2,4]triazin-6-yl)methyl)-4-methyl-1,2,5-oxadiazole-3-carboxamide (60.0 mg, 85.3 μmol, 82% yield, HCl salt) as a yellow solid. LCMS [M+H]⁺=667.3 m/z.

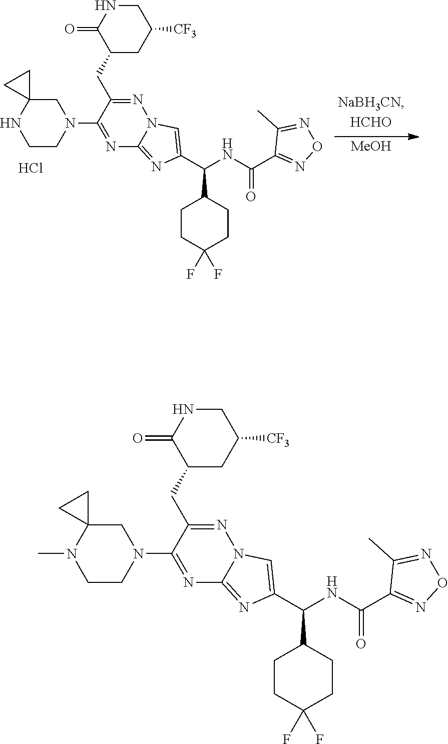 [SMILES: "MeOH" ; Cc1nonc1C(=O)N[C@H](c1cn2nc(C[C@H]3C[C@@H](C(F)(F)F)CNC3=O)c(N3CCNC4(CC4)C3)nc2n1)C1CCC(F)(F)CC1 ; Cl ; Cc1nonc1C(=O)N[C@H](c1cn2nc(C[C@H]3C[C@@H](C(F)(F)F)CNC3=O)c(N3CCN(C)C4(CC4)C3)nc2n1)C1CCC(F)(F)CC1]

To a solution of N—((S)-(4,4-difluorocyclohexyl)(2-(((3R,5R)-2-oxo-5-(trifluoromethyl)piperidin-3-yl)methyl)-3-(4,7-diazaspiro[2.5]octan-7-yl)imidazo[1,2-b][1,2,4]triazin-6-yl)methyl)-4-methyl-1,2,5-oxadiazole-3-carboxamide (60.0 mg, 85.3 μmol, 1.00 eq, HCl salt) and HCHO (12.8 mg, 426 μmol, 11.7 μL, 5.00 eq) in MeOH (2.00 mL) was added NaOAc (28.0 mg, 341 μmol, 4.00 eq) and NaBH₃CN (10.7 mg, 170 μmol, 2.00 eq) at 0° C. The mixture was stirred at 0° C. for 15 min, then the reaction mixture was diluted with sat. aq. NaHCO₃ (50.0 mL) and extracted with EtOAc (50.0 mL*3). The combined organic layers were dried over Na₂SO₄, filtered, and concentrated under reduced pressure to give a residue which was purified by Prep-HPLC (FA condition; column: Phenomenex luna C 18 150*25 mm*10 um; mobile phase: [water (FA)—ACN]; gradient: 17%-47% B over 10 min) to afford N—((S)-(4,4-difluorocyclohexyl)(3-(4-methyl-4,7-diazaspiro[2.5]octan-7-yl)-2-(((3R,5R)-2-oxo-5-(trifluoromethyl)piperidin-3-yl)methyl)imidazo[1,2-b][1,2,4]triazin-6-yl)methyl)-4-methyl-1,2,5-oxadiazole-3-carboxamide (46.39 mg, 64.1 μmol, 75% yield) as a yellow solid.

### Example 271. Preparation of N-((1S)-(3-(3-cyclopropyl-4-methylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 431 and 432)

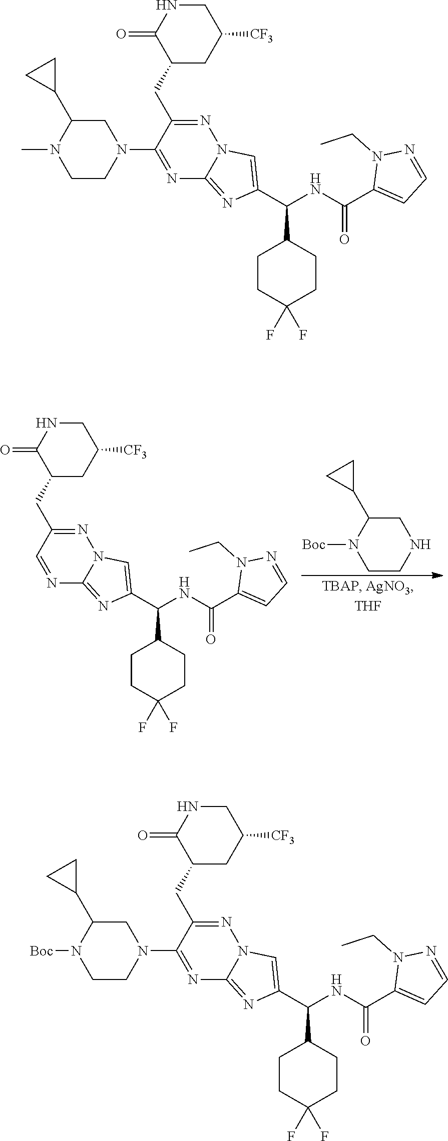 [SMILES: CCn1nccc1C(=O)N[C@H](c1cn2nc(C[C@H]3C[C@@H](C(F)(F)F)CNC3=O)c(N3CCN(C)C(C4CC4)C3)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; CC(C)(C)OC(=O)N1CCNCC1C1CC1 ; CCn1nccc1C(=O)N[C@H](c1cn2nc(C[C@H]3C[C@@H](C(F)(F)F)CNC3=O)c(N3CCN(C(=O)OC(C)(C)C)C(C4CC4)C3)nc2n1)C1CCC(F)(F)CC1]

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (120 mg, 211 μmol, 1.00 eq) with tert-butyl 2-cyclopropylpiperazine-1-carboxylate (573 mg, 2.53 mmol, 12.0 eq) was carried out following General Procedure 14, using AgNO₃ (107 mg, 633 μmol, 3.00 eq) and TBAP (457 mg, 1.27 mmol, 6.00 eq) in THF (0.1 M), stirring for 30 min at 0° C., then purifying by prep-TLC (SiO₂, EtOAc) to afford tert-butyl 2-cyclopropyl-4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperazine-1-carboxylate (107 mg, 134 μmol, 64% yield) as a yellow solid. LCMS [M+H]⁺=793.4 m/z.

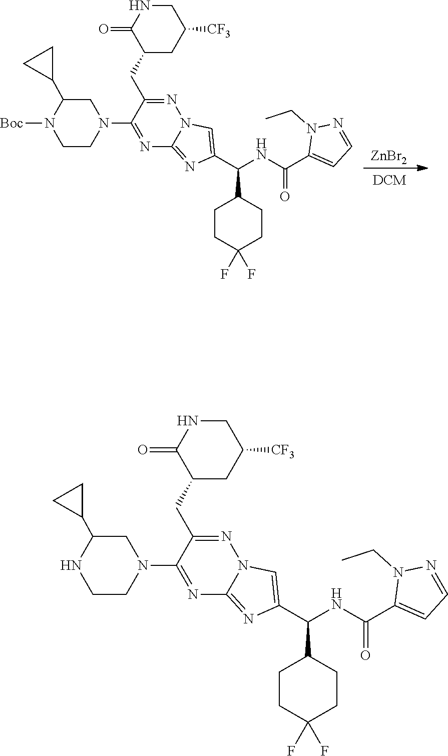

To a solution of tert-butyl 2-cyclopropyl-4-(6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)piperazine-1-carboxylate (102 mg, 128 μmol, 1.00 eq) in DCM (3.00 mL) was added ZnBr₂ (173 mg, 771 μmol, 38.6 μL, 6.00 eq). The mixture was stirred at 25° C. for 18 h. The reaction mixture was concentrated under reduced pressure to give crude N-((1 S)-(3-(3-cyclopropylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (80.0 mg, crude) as a yellow solid. LCMS [M+H]⁺=693.3 m/z.

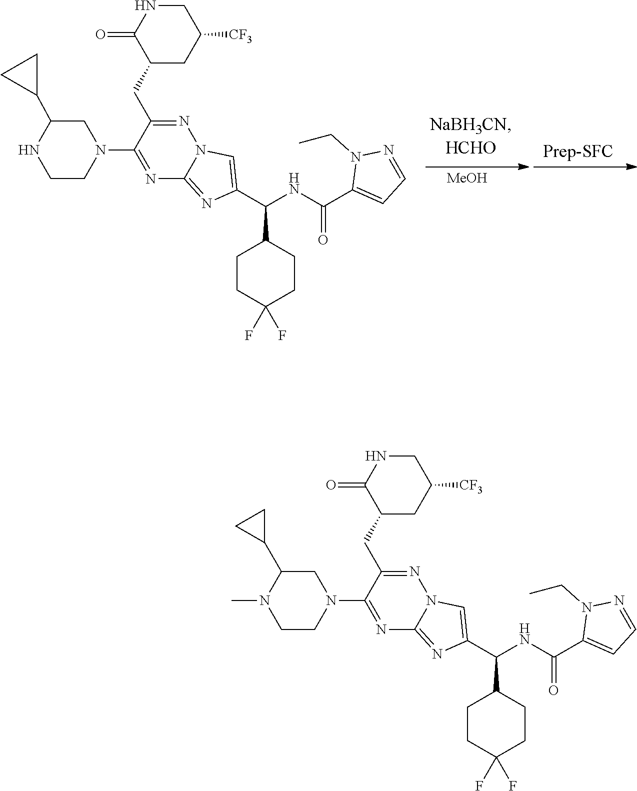 [SMILES: "MeOH" ; CCn1nccc1C(=O)N[C@H](c1cn2nc(C[C@H]3C[C@@H](C(F)(F)F)CNC3=O)c(N3CCNC(C4CC4)C3)nc2n1)C1CCC(F)(F)CC1 ; CCn1nccc1C(=O)N[C@H](c1cn2nc(C[C@H]3C[C@@H](C(F)(F)F)CNC3=O)c(N3CCN(C)C(C4CC4)C3)nc2n1)C1CCC(F)(F)CC1]

To a solution of N-((1S)-(3-(3-cyclopropylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (80.0 mg, crude) in MeOH (1.50 mL) was added formaldehyde (1.22 g, 15.0 mmol, 1.12 mL, 37% purity, 130 eq) and NaBH₃CN (21.7 mg, 346 μmol, 3.00 eq). The mixture was stirred at 25° C. for 0.5 h then filtered with MeOH washes and the filtrate concentrated under reduced pressure. The residue was purified by Prep-HPLC (column: Waters Xbridge 150*25 mm*5 um; mobile phase: [water (NH₄HCO₃)—ACN]; gradient: 35%-55% B over 10 min). The mixture of stereoisomers of N-((1S)-(3-(3-cyclopropyl-4-methylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (75.0 mg, 106 μmol, 83% yield over 2 steps) was obtained as a yellow solid.

The mixture of stereoisomers was separated by chiral SFC (column: DAICEL CHIRALCEL OD (250 mm*30 mm, 10 um); mobile phase: [CO₂-i-PrOH]; B %:35%, isocratic elution mode). The first eluting single stereoisomer of N-((1S)-(3-(3-cyclopropyl-4-methylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 431 22.16 mg, 31.0 μmol, 29% yield) was obtained as a yellow solid.

The second eluting single stereoisomer of N-((1S)-(3-(3-cyclopropyl-4-methylpiperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 432 20.8 mg, 29.0 μmol, 27% yield) was obtained as a yellow solid.

### Example 272. Preparation of N—((S)-(4,4-difluorocyclohexyl)(3-(4-(oxetan-3-yl)piperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 433)

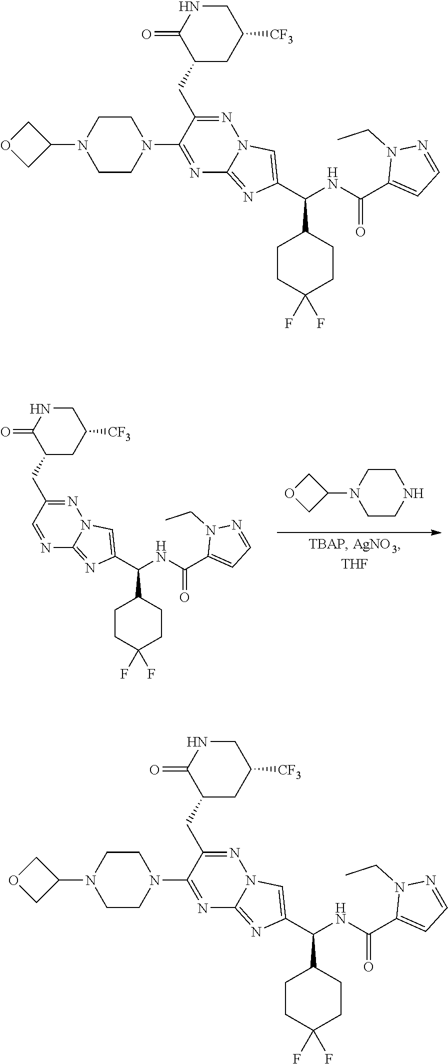

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 87.9 μmol, 1.00 eq) with 1-(oxetan-3-yl)piperazine (250 mg, 1.76 mmol, 20.0 eq) was carried out following General Procedure 14, using AgNO₃ (29.8 mg, 175 μmol, 2.00 eq) and TBAP (63.5 mg, 175 μmol, 2.00 eq) in THF (0.09 M), stirring for 1 h at 25° C., then purifying by prep-TLC (SiO₂, EtOAc/MeOH=8/1) then Prep-HPLC (column: Waters Xbridge 150*25 mm*5 um; mobile phase: [water (NH₄HCO₃)—ACN]; gradient: 25%-45% B over 10 min) to afford N—((S)-(4,4-difluorocyclohexyl)(3-(4-(oxetan-3-yl)piperazin-1-yl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (18.76 mg, 25.8 μmol, 29% yield) as a solid.

### Example 273. Preparation of N—((S)-(3-(4-(cyclopropylmethyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 244)

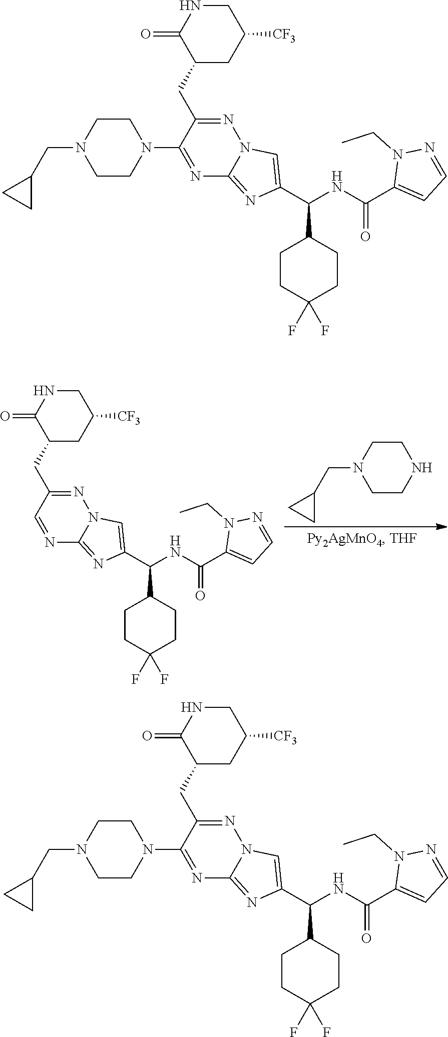

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.6 mg, 13.0 μmol, 1.00 eq) with 1-(cyclopropylmethyl)piperazine (36.4 mg, 38.6 uL, 259.5 μmol, 20.0 eq) was carried out following General Procedure 8, using bis(pyridine)silver(I) permanganate (9.1 mg, 23.6 μmol, 1.8 eq) in THF (0.03 M) and purifying by prep-HPLC (column: Gemini C18 250×21.2 mm, 5 μm; mobile phase: [water (TFA)—acetonitrile], gradient: 10%-45% B over 30 min) followed by a basic workup to afford N—((S)-(3-(4-(cyclopropylmethyl)piperazin-1-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (3.3 mg, 4.2 μmol, 32% yield) as a yellow solid.

### Example 274. Preparation of N—((S)-(3-(1,4-oxazepan-4-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 245)

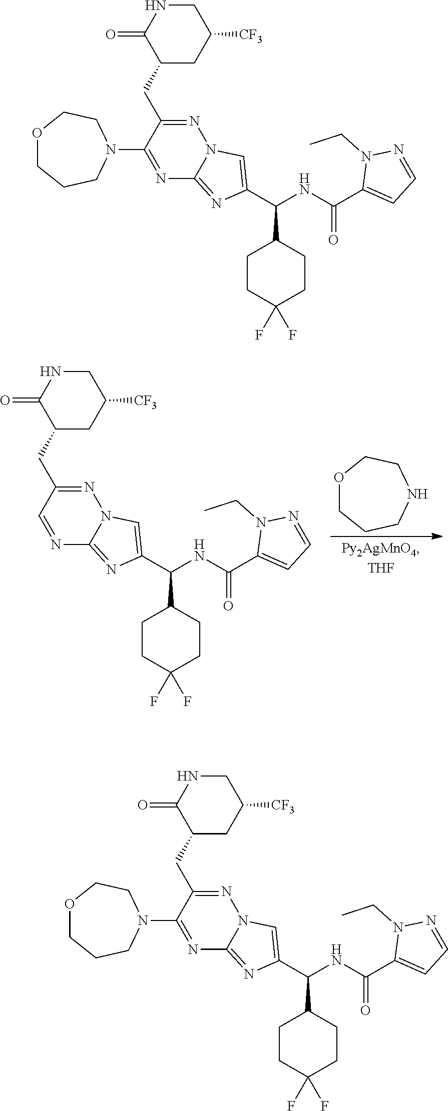

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (6.6 mg, 11.3 μmol, 1.00 eq) with 1,4-oxazepane (11.4 mg, 12.5 uL, 112.7 μmol, 10.0 eq) was carried out following General Procedure 8, using bis(pyridine)silver(I) permanganate (15.6 mg, 40.6 μmol, 3.6 eq) in THF (0.05 M) and purifying by prep-HPLC (column: Gemini C18 250×21.2 mm, 5 μm; mobile phase: [water (TFA)—acetonitrile]; gradient: 10%-50% B over 25 min) followed by a basic workup to afford N—((S)-(3-(1,4-oxazepan-4-yl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (3.04 mg, 4.3 μmol, 38% yield) as a yellow solid.

### Example 275. Preparation of N—((S)-(4,4-difluorocyclohexyl)(3-((2R,6S)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 247)

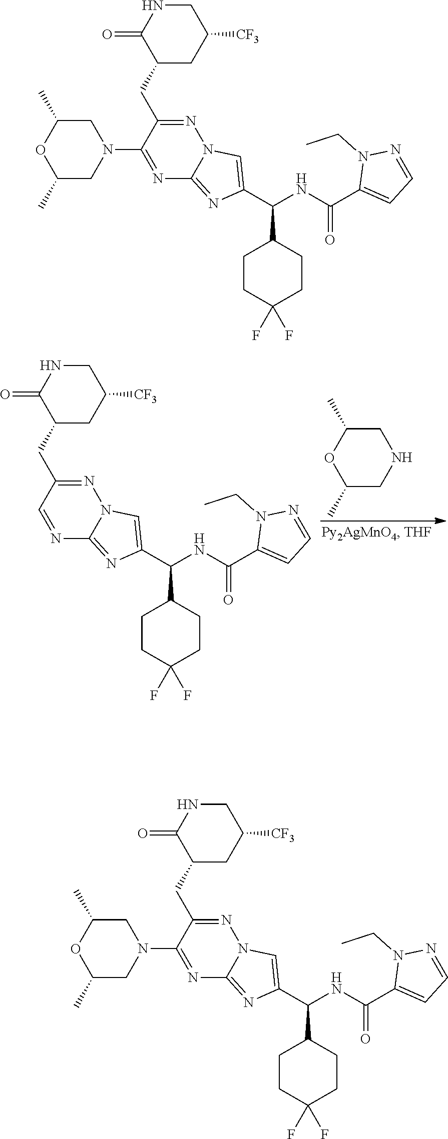

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (5.9 mg, 10.1 μmol, 1.00 eq) with (2R,6S)-2,6-dimethylmorpholine (11.6 mg, 12.4 uL, 100.7 μmol, 10.0 eq) was carried out following General Procedure 8, using bis(pyridine)silver(I) permanganate (23.9 mg, 6.2 eq) in THF (0.05 M) and purifying by prep-HPLC (column: Gemini C18 250×21.2 mm, 5 μm; mobile phase: [water (TFA)—acetonitrile], gradient: 10%-50% B over 25 min) followed by a basic workup to afford N—((S)-(4,4-difluorocyclohexyl)(3-((2R,6S)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (2.32 mg, 3.4 μmol, 34% yield) as a yellow solid.

### Example 276. Preparation of N-((1S)-(4,4-difluorocyclohexyl)(3-(2-(methoxymethyl)morpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 416)

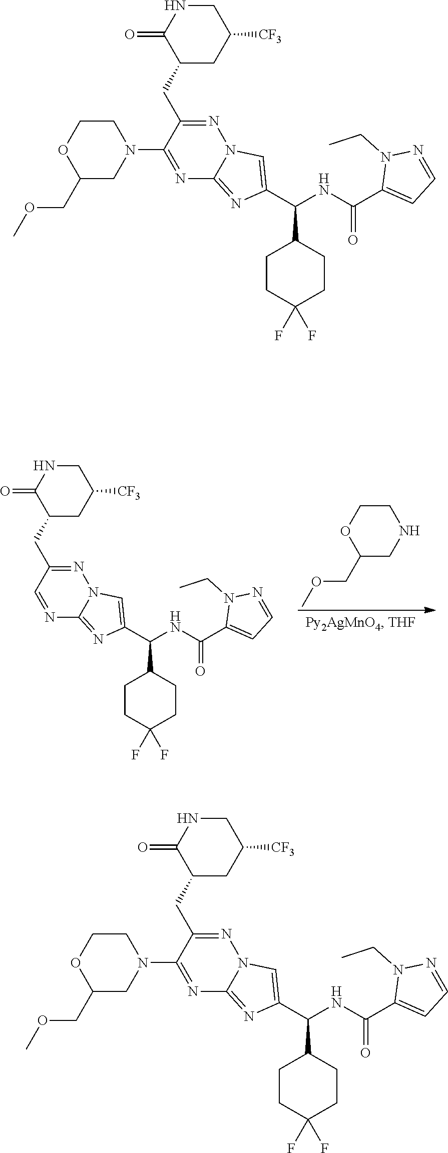

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (6.9 mg, 11.8 μmol, 1.00 eq) with 2-(methoxymethyl)morpholine (30.9 mg, 32.5 uL, 235.6 μmol, 20.0 eq) was carried out following General Procedure 8, using bis(pyridine)silver(I) permanganate (19.7 mg, 51 0.2 μmol, 4.3 eq) in THF (0.05 M) and purifying by prep-HPLC (column: Gemini C18 250×21.2 mm, 5 μm; mobile phase: [water (TFA)—acetonitrile]; gradient: 10%-56% B over 20 min) followed by a basic workup to afford N-((1 S)-(4,4-difluorocyclohexyl)(3-(2-(methoxymethyl)morpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (4.8 mg, 6.8 mg, 57% yield) as a yellow solid.

### Example 277. Preparation of N—((S)-(4,4-difluorocyclohexyl)(3-((2S,6S)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 420)

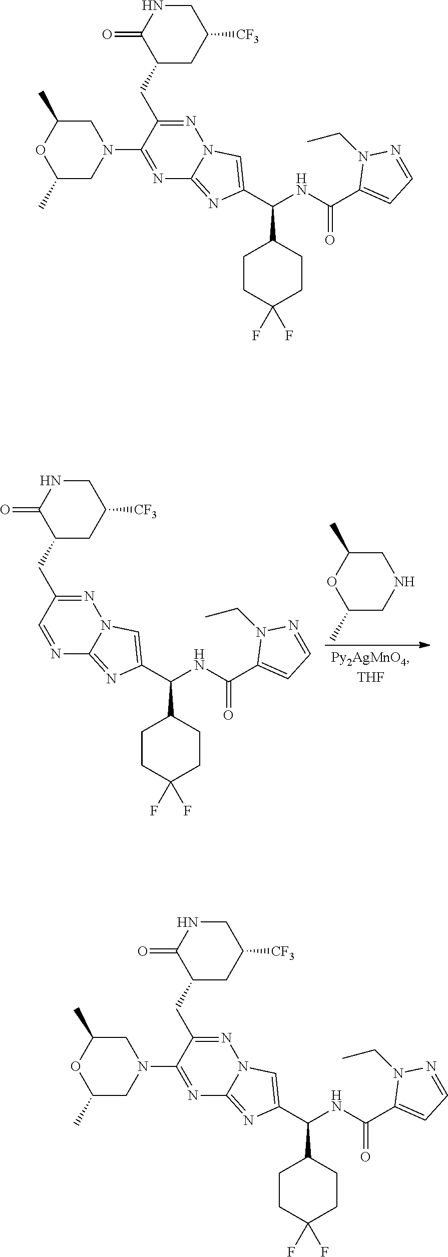

Oxidative nucleophilic substitution of hydrogen of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.1 mg, 12.1 μmol, 1.00 eq) with (2S,6S)-2,6-dimethylmorpholine (27.9 mg, 30.6 uL, 242.5 μmol, 20.0 eq) was carried out following General Procedure 8, using bis(pyridine)silver(I) permanganate (16.6 mg, 43.1 μmol, 3.6 eq) in THF (0.05 M) and purifying by prep-HPLC (column: Gemini C18 250×21.2 mm, 5 μm; mobile phase: [water (TFA)—acetonitrile], gradient: 10%-53% B over 25 min) followed by a basic workup to afford N—((S)-(4,4-difluorocyclohexyl)(3-((2S,6S)-2,6-dimethylmorpholino)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (3.03 mg, 4.3 μmol, 35% yield) as a yellow solid.

### Example 278. Preparation of N—((S)-(4,4-difluorocyclohexyl)(3-isopropoxy-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 417)

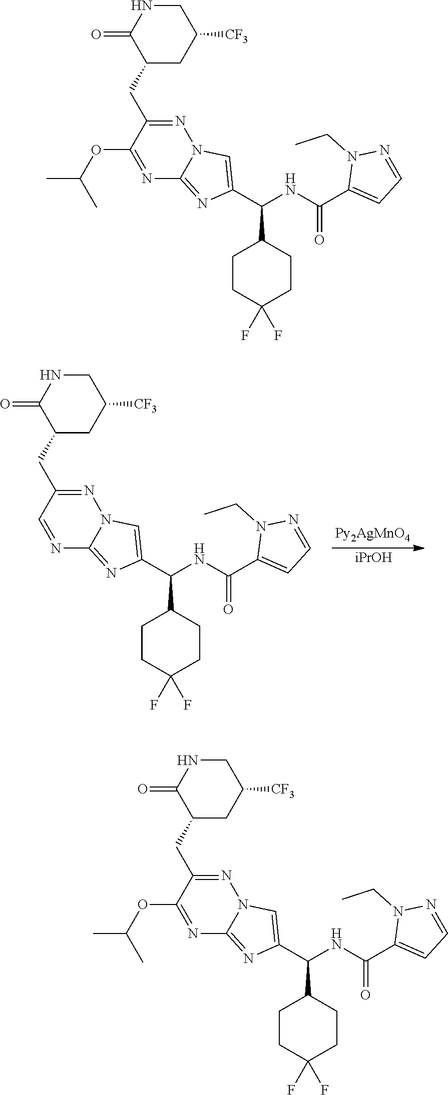

To a solution of N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (9.9 mg, 16.9 μmol, 1.00 eq) in isopropanol (0.34 mL, 0.05 M) at 5° C. was added sequential additions of bis(pyridine)silver(I) permanganate (totaling 81.6 mg, 211.9 μmol, 12.5 eq) until degradation was observed (~1 h). The reaction mixture was then diluted with DCM, filtered through packed MgSO₄ atop Celite, and concentrated to give a residue that was purified by prep-HPLC (column: Gemini C18 250×21.2 mm, 5 μm; mobile phase: [water (TFA)—acetonitrile]; gradient: 10%-60% B over 40 min) and lyophilized directly to afford N—((S)-(4,4-difluorocyclohexyl)(3-isopropoxy-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (0.55 mg, 0.7 μmol, 4% yield, TFA salt).

### Example 279. Preparation of N—((S)-(4,4-difluorocyclohexyl)(3-((methylamino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 286)

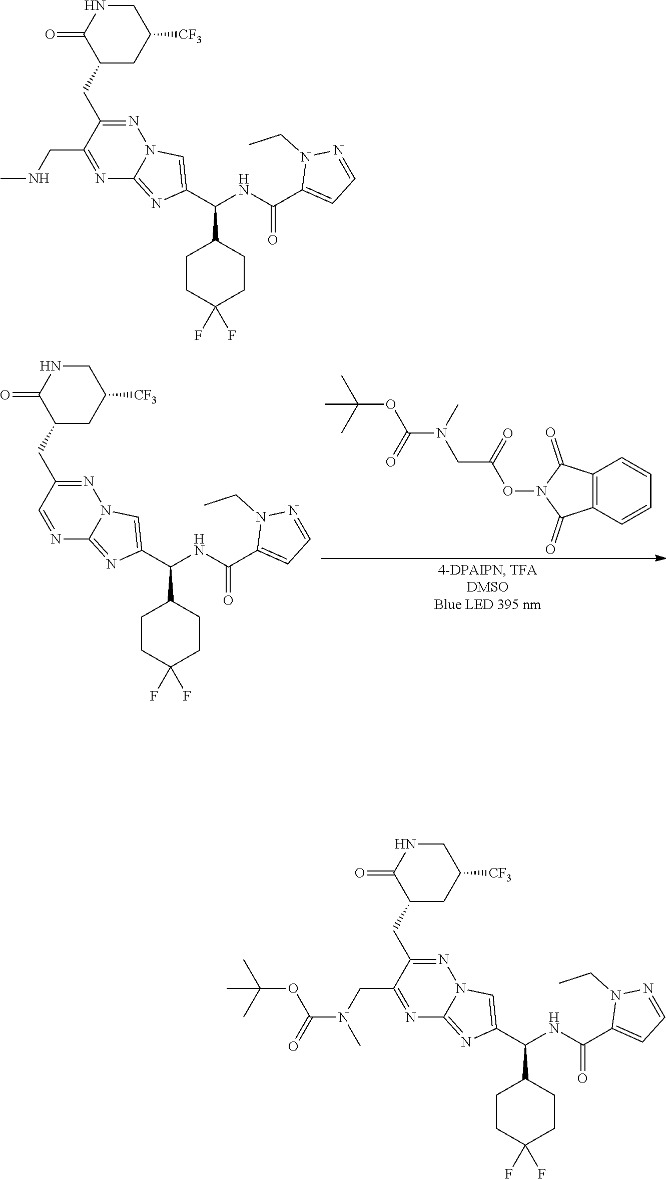 [SMILES: CCn1nccc1C(=O)N[C@H](c1cn2nc(C[C@H]3C[C@@H](C(F)(F)F)CNC3=O)c(CNC)nc2n1)C1CCC(F)(F)CC1 ; CCn1nccc1C(=O)N[C@H](c1cn2nc(C[C@H]3C[C@@H](C(F)(F)F)CNC3=O)cnc2n1)C1CCC(F)(F)CC1 ; CN(CC(=O)ON1C(=O)c2ccccc2C1=O)C(=O)OC(C)(C)C ; CCn1nccc1C(=O)N[C@H](c1cn2nc(C[C@H]3C[C@@H](C(F)(F)F)CNC3=O)c(CN(C)C(=O)OC(C)(C)C)nc2n1)C1CCC(F)(F)CC1]

Intermediate tert-butyl ((6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)methyl)(methyl)carbamate was synthesized following General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (150 mg, 263 μmol, 1.00 eq) and 1,3-dioxoisoindolin-2-yl N-(tert-butoxycarbonyl)-N-methylglycinate (176 mg, 527 μmol, 2.00 eq) and was purified by Prep-TLC (SiO₂, DCM/MeOH=10:1) then Prep-HPLC (condition: column: Waters Xbridge 150*25 mm*5 um; mobile phase: [water (NH₄HCO₃)—ACN]; gradient: 35%-55% B over 53 min) to afford tert-butyl ((6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)methyl)(methyl)carbamate (60.0 mg, 84.3 μmol, 32% yield) as a yellow solid. LCMS [M+H]⁺=712.6 m/z.

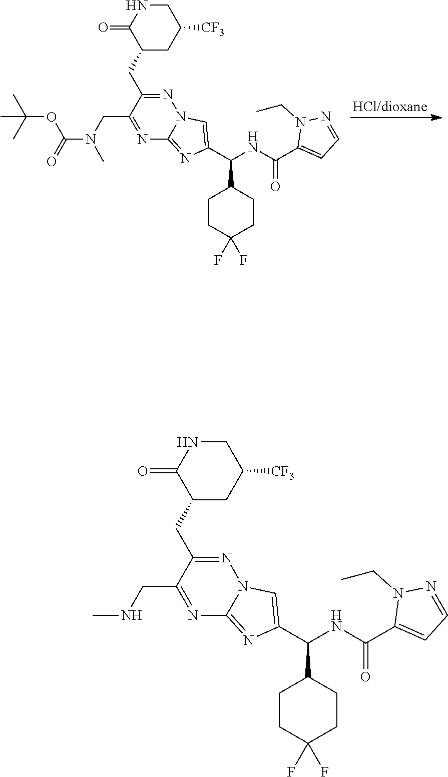 [SMILES: CCn1nccc1C(=O)N[C@H](c1cn2nc(C[C@H]3C[C@@H](C(F)(F)F)CNC3=O)c(CN(C)C(=O)OC(C)(C)C)nc2n1)C1CCC(F)(F)CC1 ; CCn1nccc1C(=O)N[C@H](c1cn2nc(C[C@H]3C[C@@H](C(F)(F)F)CNC3=O)c(CNC)nc2n1)C1CCC(F)(F)CC1]

To a solution of tert-butyl ((6-((S)-(4,4-difluorocyclohexyl)(1-ethyl-1H-pyrazole-5-carboxamido)methyl)-2-(((3R,5R)-2-oxo-5-(trifluoromethyl)piperidin-3-yl)methyl)imidazo[1,2-b][1,2,4]triazin-3-yl)methyl)(methyl)carbamate (55.0 mg, 77.2 μmol, 1.00 eq) in DCM (1.00 mL) was added HCl/dioxane (2 M, 386 μL, 10.0 eq) at 0° C. The mixture was stirred at 25° C. for 0.5 h. The reaction mixture was concentrated under reduced pressure to remove HCl/dioxane and DCM and afford N—((S)-(4,4-difluorocyclohexyl)(3-((methylamino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.63 mg, 51.5 μmol, HCl salt) as a yellow solid.

### Example 280. Preparation of N—((S)-(4,4-difluorocyclohexyl)(2-(((3R,5R)-2-oxo-5-(trifluoromethyl)piperidin-3-yl)methyl)-3-(piperidin-1-ylmethyl)imidazo[1,2-b][1,2,4]triazin-6-yl)methyl)-1-ethyl-1H-pyrazole-5-carboxamide (Compound 288)

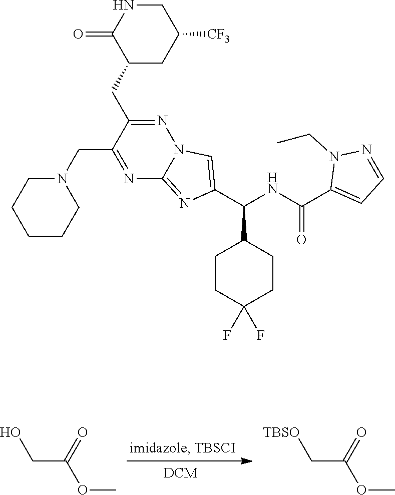

To a solution of methyl 2-hydroxyacetate (15.0 g, 166 mmol, 12.8 mL, 1.00 eq) in DMF (150 mL) was added imidazole (28.3 g, 416 mmol, 2.50 eq) and TBSC1 (27.6 g, 183 mmol, 22.5 mL, 1.10 eq). The mixture was stirred at 25° C. for 3 h. The reaction mixture was then diluted with H₂O (30.0 mL) and extracted with Pet. Ether (30.0 mL*3). The combined organic layers were dried over Na₂SO₄, filtered, and concentrated under reduced pressure to give a residue, which was used in the next step directly without further purification. Methyl 2-((tert-butyldimethylsilyl)oxy)acetate (30.0 g, 146 mmol, 88% yield) was obtained as a colorless oil. ¹H NMR (400 MHz, CDCl₃) δ 4.26-4.19 (m, 2H), 3.77-3.62 (m, 3H), 0.92-0.87 (m, 9H), 0.11-0.05 (m, 6H).

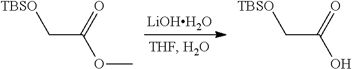

To a solution of methyl 2-((tert-butyldimethylsilyl)oxy)acetate (15.0 g, 73.4 mmol, 1.00 eq) in THF (125 mL) at 0° C. was added H₂O (25.0 mL) and LiOH·H₂O (6.16 g, 146 mmol, 2.00 eq). The mixture was stirred at 25° C. for 3 h, then diluted with H₂O (100 mL) and added 1 M HCl to adjust pH to 3. The mixture was further diluted with H₂O (200 mL) and extracted with dichloromethane (100 mL*3). The combined organic layers were dried over Na₂SO₄, filtered, and concentrated under reduced pressure to give a residue, which was used to next step directly without any purification. The product, 2-((tert-butyldimethylsilyl)oxy)acetic acid (10.0 g, 52.5 mmol, 72% yield), was obtained as a colourless oil. ¹H NMR (400 MHz, CDCl₃) δ 4.34-4.13 (m, 2H), 0.93 (s, 9H), 0.13 (s, 6H).

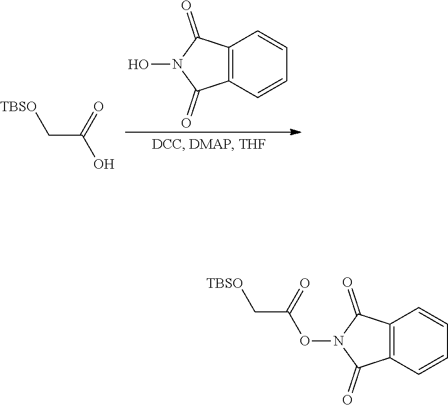 [SMILES: CC(C)(C)[Si](C)(C)OCC(=O)O ; O=C1c2ccccc2C(=O)N1O ; CC(C)(C)[Si](C)(C)OCC(=O)ON1C(=O)c2ccccc2C1=O]

To a solution of 2-((tert-butyldimethylsilyl)oxy)acetic acid (3.00 g, 15.76 mmol, 1.00 eq) and 2-hydroxyisoindoline-1,3-dione (3.21 g, 19.7 mmol, 1.25 eq) in THF (50.0 mL) was added DCC (3.58 g, 17.3 mmol, 3.51 mL, 1.10 eq) and DMAP (192 mg, 1.58 mmol, 0.100 eq). The mixture was stirred at 25° C. for 12 h. The reaction mixture was then diluted with H₂O (30.0 mL) and extracted with EtOAc (30.0 mL*3). The combined organic layers were washed with sat. aq. NaHCO₃ (30.0 mL*2), dried over Na₂SO₄, filtered, and concentrated under reduced pressure to give a residue. The residue was purified by column chromatography (SiO₂, Pet Ether:EtOAc=1:0 to 3:1) to afford 1,3-dioxoisoindolin-2-yl 2-((tert-butyldimethylsilyl)oxy)acetate (5.00 g, 14.9 mmol, 95% yield) as a white solid. H NMR (400 MHz, CDCl₃) δ 8.00-7.70 (m, 4H), 4.65 (s, 2H), 0.93 (s, 9H), 0.15 (s, 6H).

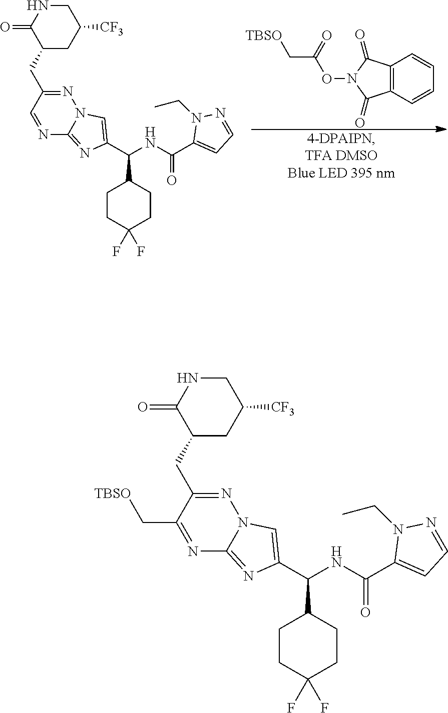 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(C)(C)[Si](C)(C)OCC(=O)ON1C(=O)c2ccccc2C1=O ; "4-DPAIPN,TFA DMSOBlue LED 395 nm" ; CCn1nccc1C(=O)N[C@H](c1cn2nc(C[C@H]3C[C@@H](C(F)(F)F)CNC3=O)c(CO[Si](C)(C)C(C)(C)C)nc2n1)C1CCC(F)(F)CC1]

Intermediate N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (350 mg, 615 μmol, 1.00 eq) and 1,3-dioxoisoindolin-2-yl 2-((tert-butyldimethylsilyl)oxy)acetate (247 mg, 738 μmol, 1.20 eq) and was purified by Prep-TLC (SiO₂, Pet Ether/EtOAc=1:1) to afford N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (200 mg, 280 μmol, 46% yield) as a yellow solid. LCMS [M+H]⁺=713.4 m/z.

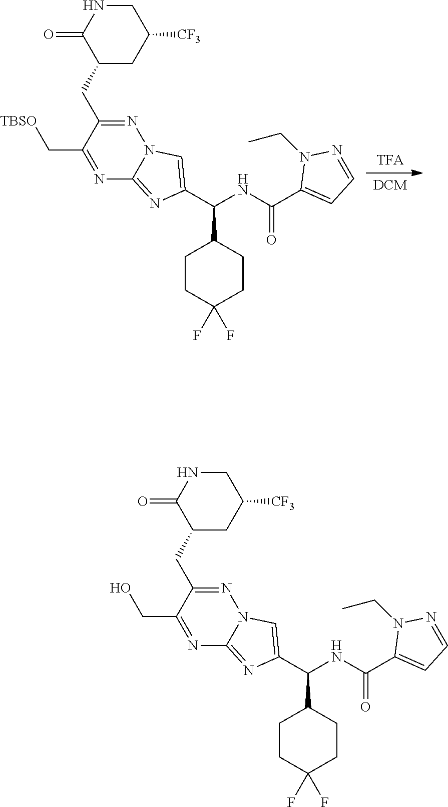 [SMILES: CCn1nccc1C(=O)N[C@H](c1cn2nc(C[C@H]3C[C@@H](C(F)(F)F)CNC3=O)c(CO[Si](C)(C)C(C)(C)C)nc2n1)C1CCC(F)(F)CC1 ; CCn1nccc1C(=O)N[C@H](c1cn2nc(C[C@H]3C[C@@H](C(F)(F)F)CNC3=O)c(CO)nc2n1)C1CCC(F)(F)CC1]

To a solution of N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (200 mg, 280 μmol, 1.00 eq) in DCM (3.00 mL) was added TFA (639 mg, 5.61 mmol, 416 μL, 20.0 eq) at 0° C. The mixture was stirred at 25° C. for 1 h then concentrated under vacuum. The reaction mixture was then basified with NaHCO₃ to pH=8 and extracted with EtOAc (20.0 mL*3). The combined organic layers were dried over anhydrous Na₂SO₄, filtered, and concentrated under reduced pressure to give a yellow solid, N—((S)-(4,4-difluorocyclohexyl)(3-(hydroxymethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (130 mg, 217 μmol, 77% yield), which was used in the next step directly without further purification. LCMS [M+H]⁺=599.3 m/z.

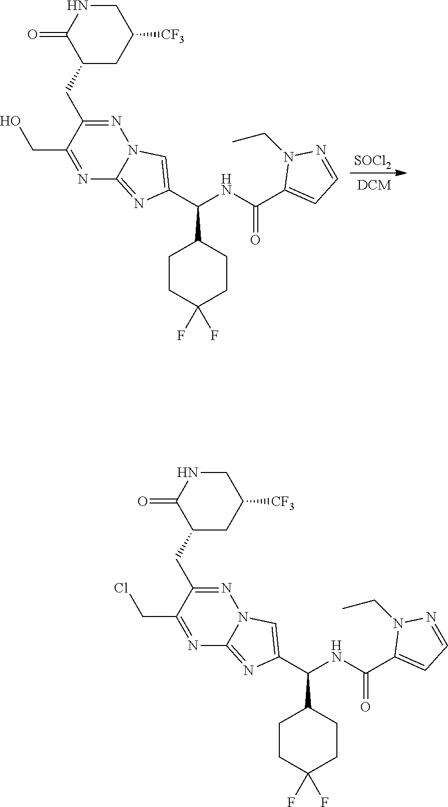

To a solution of N—((S)-(4,4-difluorocyclohexyl)(3-(hydroxymethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (130 mg, 217 μmol, 1.00 eq) in DCM (2.00 mL) was added SOCl₂ (77.5 mg, 651 μmol, 47.3 μL, 3.00 eq) at 0° C. The mixture was stirred at 25° C. for 1 h then concentrated under reduced pressure to give crude N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (120 mg, 194 μmol, 89.5% yield) as a yellow solid, which was used in the next step directly without further purification. LCMS [M+H]⁺=617.4 m/z.

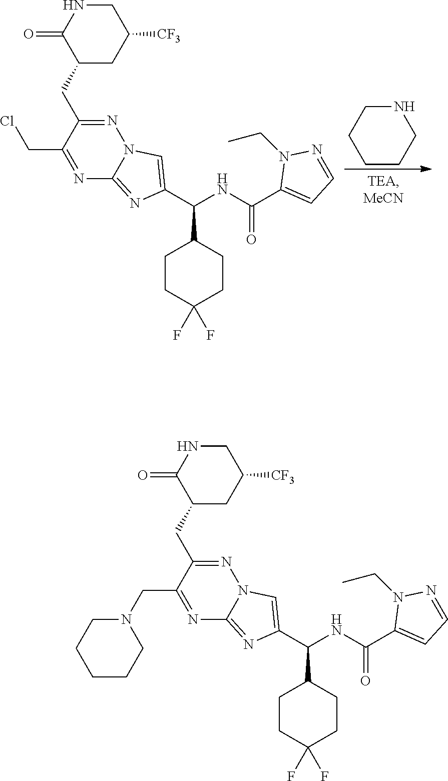

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide ( . . . ) and piperidine hydrochloride (9.46 mg, 77.7 μmol, 10.9 μL, 1.20 eq, HCl salt). Purification by Prep-HPLC (column: Phenomenex luna C18 150*25 mm*10 um; mobile phase: [water (FA)—ACN]; gradient: 18%-48% B over 10 min) afforded N—((S)-(4,4-difluorocyclohexyl)(2-(((3R,5R)-2-oxo-5-(trifluoromethyl)piperidin-3-yl)methyl)-3-(piperidin-1-ylmethyl)imidazo[1,2-b][1,2,4]triazin-6-yl)methyl)-1-ethyl-1H-pyrazole-5-carboxamide (6.47 mg, 9.15 μmol, 14% yield) as a yellow solid.

### Example 281. Preparation of N—((S)-(4,4-difluorocyclohexyl)(3-((dimethylamino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 287)

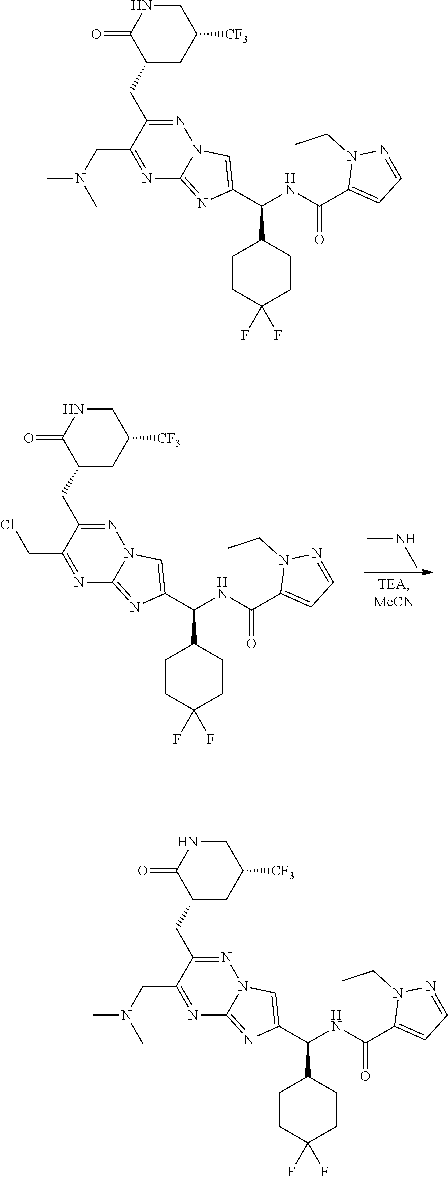

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (30.0 mg, 48.6 μmol, 1.00 eq) and dimethylamine hydrochloride (4.76 mg, 58.3 μmol, 1.20 eq, HCl salt). Purification by Prep-TLC (SiO₂, DCM/MeOH=100:1 to 10:1) afforded N—((S)-(4,4-difluorocyclohexyl)(3-((dimethylamino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (13.61 mg, 20.4 μmol, 42% yield) as a yellow solid.

### Example 282. Preparation of N—((S)-(3-((3,3-difluoroazetidin-1-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 332)

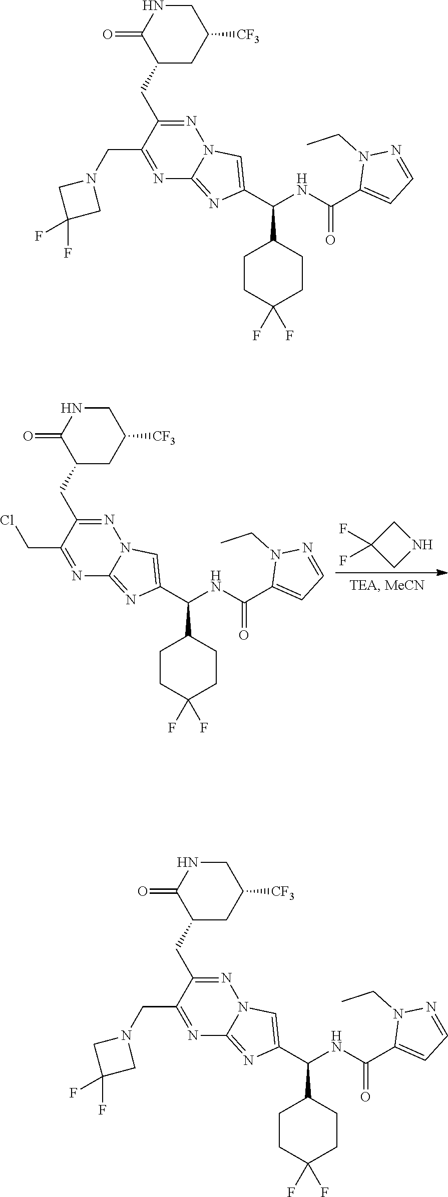

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 3,3-difluoroazetidine hydrochloride (10.0 mg, 77.7 μmol, 1.20 eq, HCl salt). Purification by Prep-HPLC (FA condition, column: Phenomenex Luna C18 150*25 mm*10 um; mobile phase: [water (FA)—ACN]; gradient: 29%-59% B over 10 min) afforded N—((S)-(3-((3,3-difluoroazetidin-1-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (13.22 mg, 18.6 μmol, 29% yield) as a yellow solid.

### Example 283. Preparation of N—((S)-(4,4-difluorocyclohexyl)(3-((3,3-difluoropyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 333)

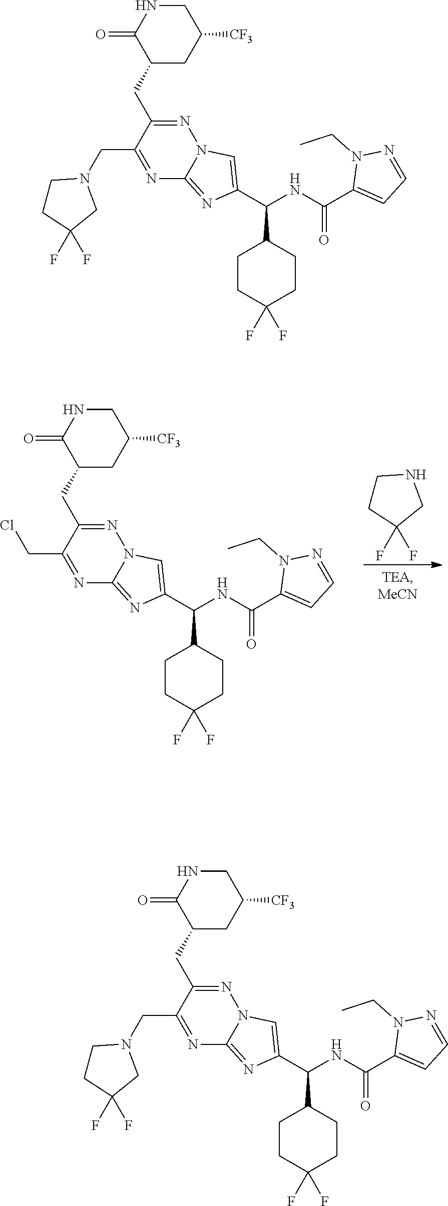 [SMILES: CCn1nccc1C(=O)N[C@H](c1cn2nc(C[C@H]3C[C@@H](C(F)(F)F)CNC3=O)c(CN3CCC(F)(F)C3)nc2n1)C1CCC(F)(F)CC1 ; CCn1nccc1C(=O)N[C@H](c1cn2nc(C[C@H]3C[C@@H](C(F)(F)F)CNC3=O)c(CCl)nc2n1)C1CCC(F)(F)CC1 ; FC1(F)CCNC1 ; CCn1nccc1C(=O)N[C@H](c1cn2nc(C[C@H]3C[C@@H](C(F)(F)F)CNC3=O)c(CN3CCC(F)(F)C3)nc2n1)C1CCC(F)(F)CC1]

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 3,3-difluoropyrrolidine (8.33 mg, 77.7 μmol, 1.20 eq, HCl salt). Purification by Prep-TLC (SiO₂, EtOAc/MeOH=20:1) afforded N—((S)-(4,4-difluorocyclohexyl)(3-((3,3-difluoropyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (9.10 mg, 12.1 μmol, 19% yield) as a yellow solid.

### Example 284. Preparation of N—((S)-(4,4-difluorocyclohexyl)(2-(((3R,5R)-2-oxo-5-(trifluoromethyl)piperidin-3-yl)methyl)-3-(((2,2,2-trifluoroethyl)amino)methyl)imidazo[1,2-b][1,2,4]triazin-6-yl)methyl)-1-ethyl-1H-pyrazole-5-carboxamide (Compound 331)

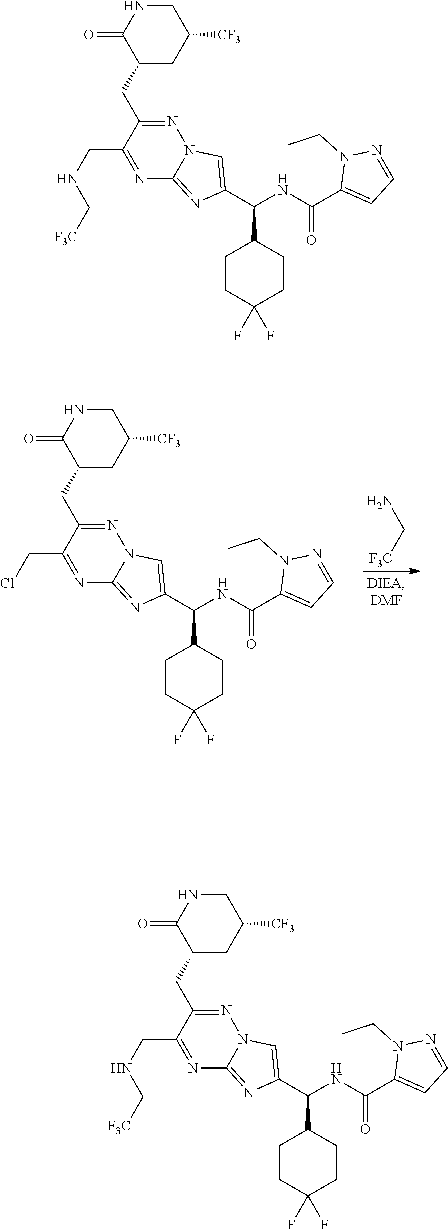 [SMILES: CCn1nccc1C(=O)N[C@H](c1cn2nc(C[C@H]3C[C@@H](C(F)(F)F)CNC3=O)c(CNCC(F)(F)F)nc2n1)C1CCC(F)(F)CC1 ; CCn1nccc1C(=O)N[C@H](c1cn2nc(C[C@H]3C[C@@H](C(F)(F)F)CNC3=O)c(CCl)nc2n1)C1CCC(F)(F)CC1 ; NCC(F)(F)F ; CCn1nccc1C(=O)N[C@H](c1cn2nc(C[C@H]3C[C@@H](C(F)(F)F)CNC3=O)c(CNCC(F)(F)F)nc2n1)C1CCC(F)(F)CC1]

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (30.0 mg, 48.6 μmol, 1.00 eq) and 2,2,2-trifluoroethan-1-amine (24.1 mg, 243 μmol, 19.1 μL, 5.00 eq). Purification by Prep-TLC (SiO₂, DCM/MeOH=10:1) afforded N—((S)-(4,4-difluorocyclohexyl)(2-(((3R,5R)-2-oxo-5-(trifluoromethyl)piperidin-3-yl)methyl)-3-(((2,2,2-trifluoroethyl)amino)methyl)imidazo[1,2-b][1,2,4]triazin-6-yl)methyl)-1-ethyl-1H-pyrazole-5-carboxamide (7.44 mg, 10.6 μmol, 22% yield) as a white solid.

### Example 285. Preparation of N—((S)-(3-((1H-imidazol-1-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 334)

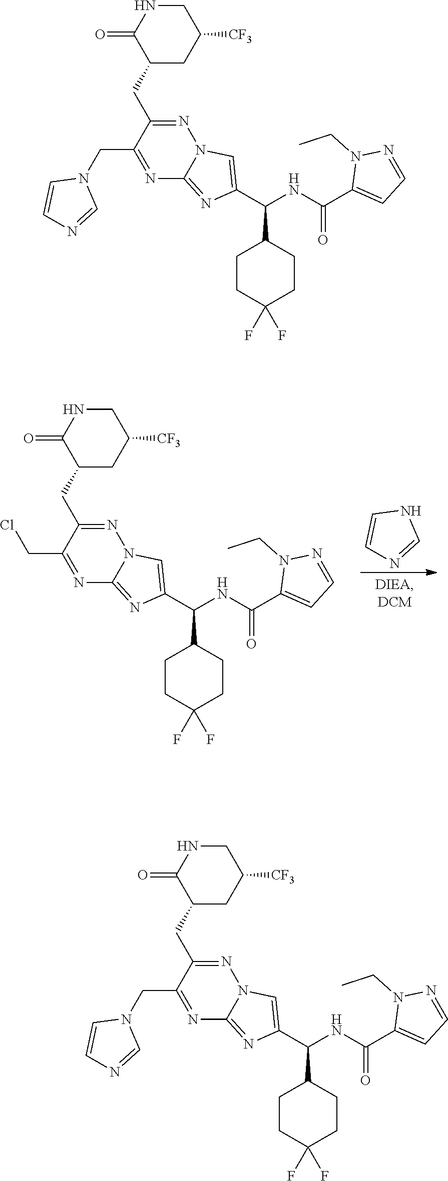 [SMILES: CCn1nccc1C(=O)N[C@H](c1cn2nc(C[C@H]3C[C@@H](C(F)(F)F)CNC3=O)c(Cn3ccnc3)nc2n1)C1CCC(F)(F)CC1 ; CCn1nccc1C(=O)N[C@H](c1cn2nc(C[C@H]3C[C@@H](C(F)(F)F)CNC3=O)c(CCl)nc2n1)C1CCC(F)(F)CC1 ; c1c[nH]cn1 ; CCn1nccc1C(=O)N[C@H](c1cn2nc(C[C@H]3C[C@@H](C(F)(F)F)CNC3=O)c(Cn3ccnc3)nc2n1)C1CCC(F)(F)CC1]

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 1H-imidazole (16.5 mg, 243 μmol, 3.00 eq). Purification by Prep-TLC (SiO₂, EtOAc/MeOH=20:1) afforded N—((S)-(3-((1H-imidazol-1-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (7.68 mg, 11.8 μmol, 15% yield) as a white solid.

### Example 286. Preparation of N—((S)-(4,4-difluorocyclohexyl)(3-((methyl(2,2,2-trifluoroethyl)amino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 335)

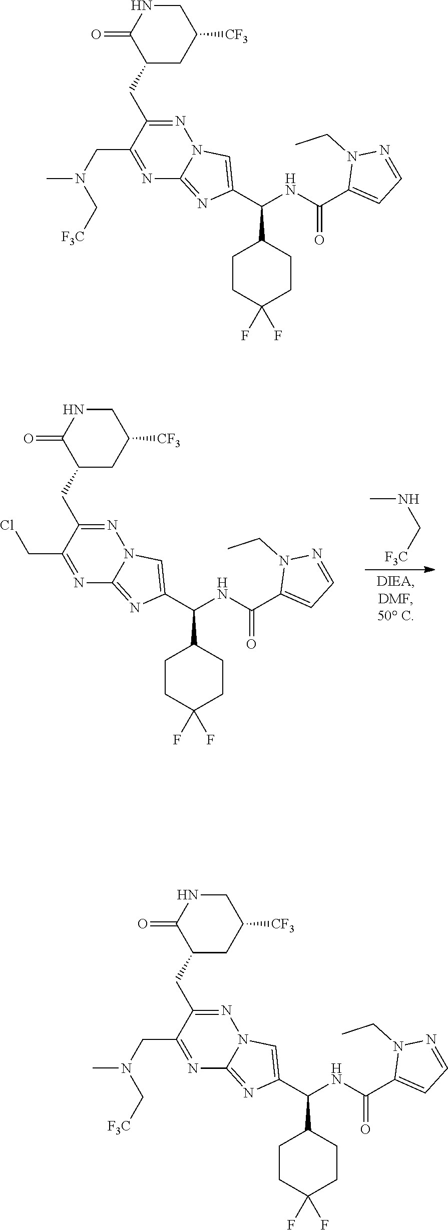

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 2,2,2-trifluoro-N-methylethan-1-amine hydrochloride (36.3 mg, 243 μmol, 3.00 eq, HCl salt). Purification by Prep-TLC (SiO₂, EtOAc/MeOH=20:1) afforded N—((S)-(4,4-difluorocyclohexyl)(3-((methyl(2,2,2-trifluoroethyl)amino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (8.00 mg, 11.3 μmol, 14% yield) as a yellow solid.

### Example 287. Preparation of N—((S)-(4,4-difluorocyclohexyl)(2-(((3R,5R)-2-oxo-5-(trifluoromethyl)piperidin-3-yl)methyl)-3-((3-(trifluoromethyl)azetidin-1-yl)methyl)imidazo[1,2-b][1,2,4]triazin-6-yl)methyl)-1-ethyl-1H-pyrazole-5-carboxamide (Compound 336)

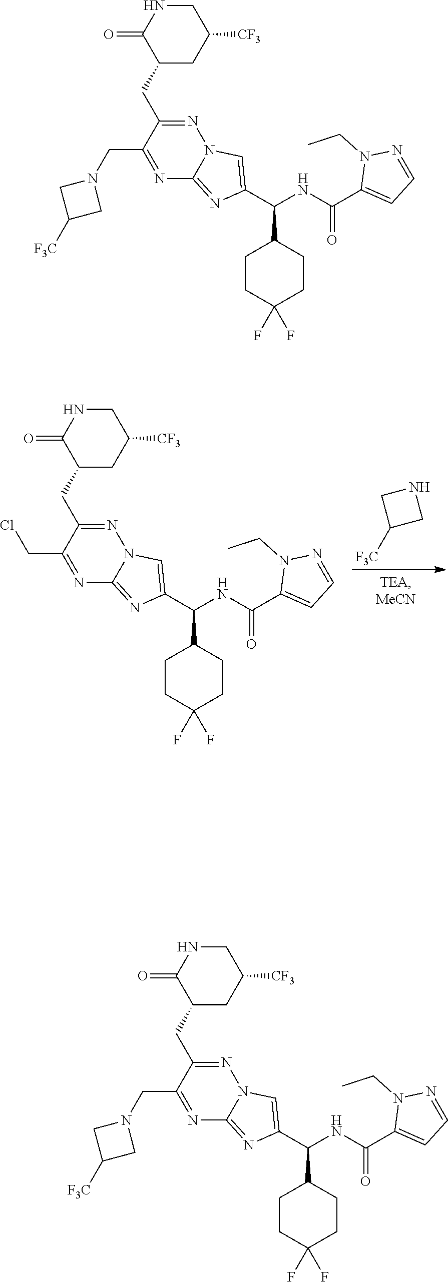 [SMILES: CCn1nccc1C(=O)N[C@H](c1cn2nc(C[C@H]3C[C@@H](C(F)(F)F)CNC3=O)c(CN3CC(C(F)(F)F)C3)nc2n1)C1CCC(F)(F)CC1 ; CCn1nccc1C(=O)N[C@H](c1cn2nc(C[C@H]3C[C@@H](C(F)(F)F)CNC3=O)c(CCl)nc2n1)C1CCC(F)(F)CC1 ; FC(F)(F)C1CNC1 ; CCn1nccc1C(=O)N[C@H](c1cn2nc(C[C@H]3C[C@@H](C(F)(F)F)CNC3=O)c(CN3CC(C(F)(F)F)C3)nc2n1)C1CCC(F)(F)CC1]

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (35.0 mg, 56.7 μmol, 1.00 eq) and 3-(trifluoromethyl)azetidine hydrochloride (8.52 mg, 68.0 μmol, 1.20 eq, HCl salt). Purification by Prep-TLC (SiO₂, EtOAc/MeOH=20:1) afforded N—((S)-(4,4-difluorocyclohexyl)(2-(((3R,5R)-2-oxo-5-(trifluoromethyl)piperidin-3-yl)methyl)-3-((3-(trifluoromethyl)azetidin-1-yl)methyl)imidazo[1,2-b][1,2,4]triazin-6-yl)methyl)-1-ethyl-1H-pyrazole-5-carboxamide (15.44 mg, 21.2 μmol, 38% yield) as a white solid.

### Example 288. Preparation of N—((S)-(4,4-difluorocyclohexyl)(3-((3-fluoro-3-methylazetidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 337)

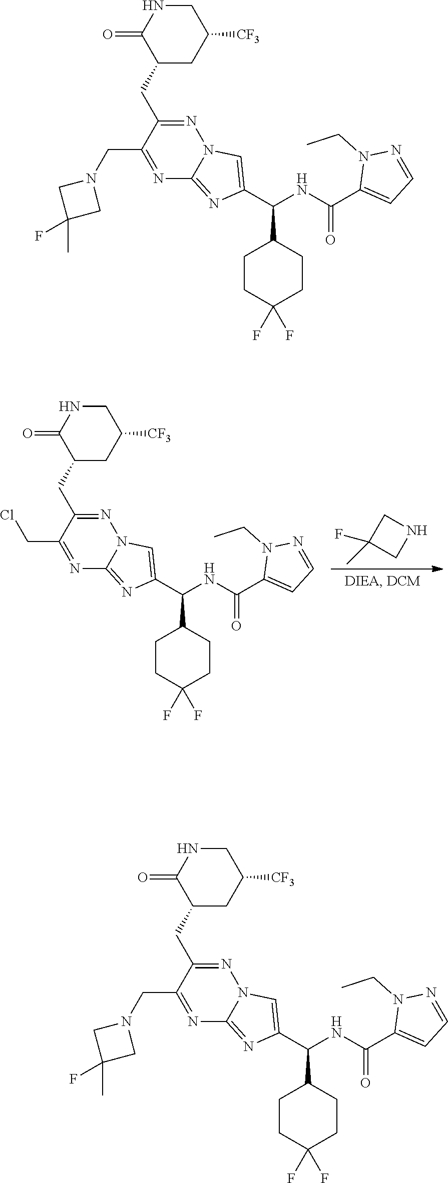

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 3-fluoro-3-methylazetidine hydrochloride (16.3 mg, 129 μmol, 2.00 eq, HCl salt). Purification by prep-TLC (SiO₂, EtOAc) afforded N—((S)-(4,4-difluorocyclohexyl)(3-((3-fluoro-3-methylazetidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (25.0 mg, 35.8 μmol, 55% yield) as a white solid.

### Example 289. Preparation of N-((1S)-(4,4-difluorocyclohexyl)(3-((2,4-dimethylazetidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 338)

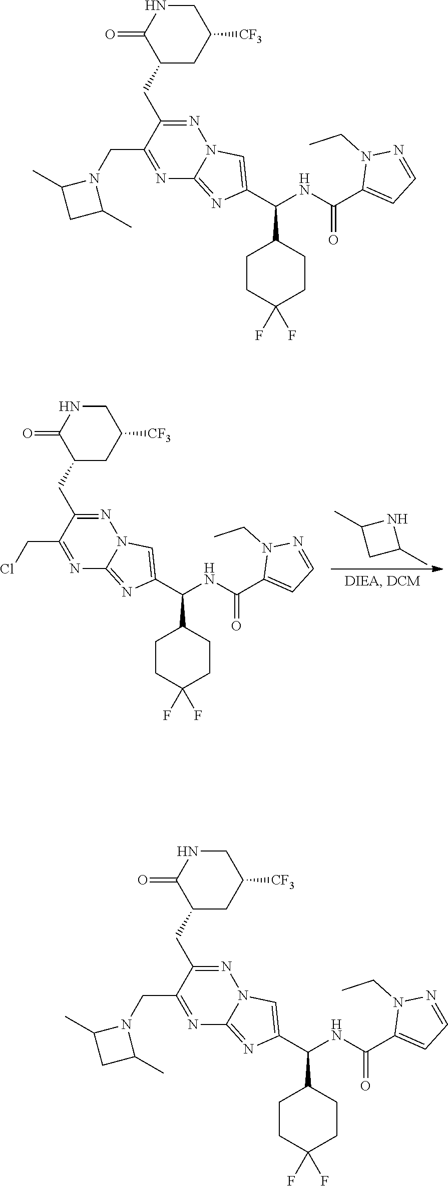

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (45.0 mg, 72.9 μmol, 1.00 eq) and 2,4-dimethylazetidine hydrochloride (17.7 mg, 146 μmol, 2.00 eq, HCl salt). Purification by prep-TLC (SiO₂, EtOAc) afforded N-((1S)-(4,4-difluorocyclohexyl)(3-((2,4-dimethylazetidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (10.0 mg, 14.6 μmol, 20.1% yield) as a white solid.

### Example 290. Preparation of N—((S)-(3-((5-azaspiro[2.3]hexan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 339)

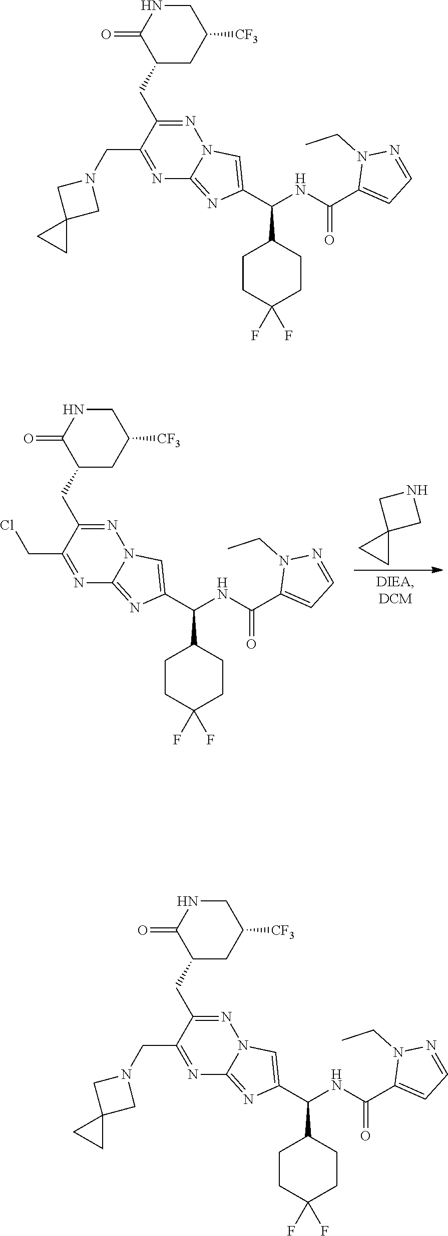

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (38.0 mg, 61.5 μmol, 1.00 eq) and 5-azaspiro[2.3]hexane hydrochloride (11.0 mg, 92.8 μmol, 1.50 eq, HCl salt). Purification by Prep-TLC (SiO₂, EtOAc/MeOH=20:1) afforded N—((S)-(3-((5-azaspiro[2.3]hexan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (18.57 mg, 25.2 μmol, 41% yield) as a white solid.

### Example 291. Preparation of N—((S)-(3-((6,6-difluoro-2-azaspiro[3.3]heptan-2-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 340)

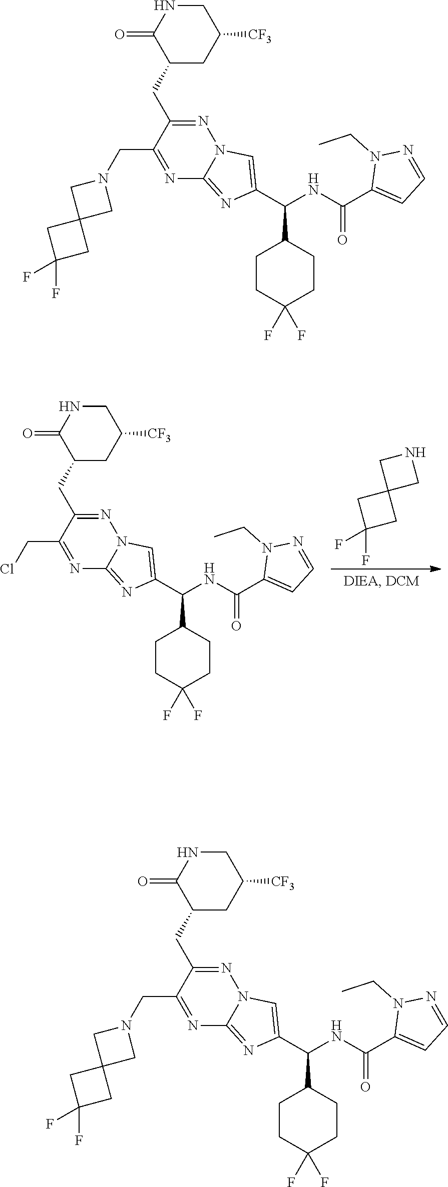

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (38.0 mg, 61.6 μmol, 1.00 eq) and 6,6-difluoro-2-azaspiro[3.3]heptane hydrochloride (20.8 mg, 123 μmol, 2.00 eq, HCl salt). Purification by Prep-TLC (SiO₂, EtOAc/MeOH=20:1) afforded N—((S)-(3-((6,6-difluoro-2-azaspiro[3.3]heptan-2-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (9.26 mg, 12.3 μmol, 20% yield) as a yellow solid.

### Example 292. Preparation of N—((S)-(4,4-difluorocyclohexyl)(3-((6-hydroxy-6-(trifluoromethyl)-2-azaspiro[3.3]heptan-2-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 341)

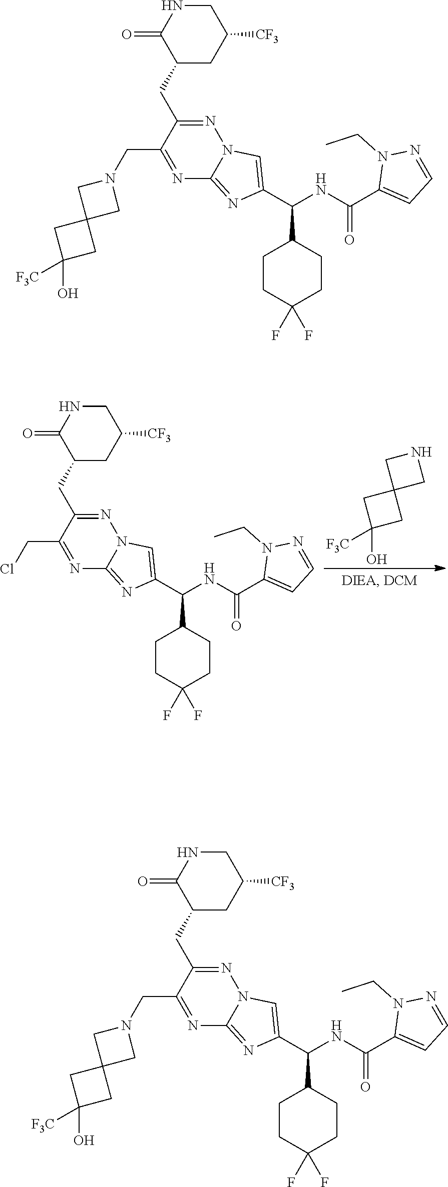

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 6-(trifluoromethyl)-2-azaspiro[3.3]heptan-6-ol hydrochloride (28.2 mg, 129 μmol, 2.00 eq, HCl salt). Purification by Prep-TLC (SiO₂, EtOAc/MeOH=20:1) afforded N—((S)-(4,4-difluorocyclohexyl)(3-((6-hydroxy-6-(trifluoromethyl)-2-azaspiro[3.3]heptan-2-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (9.39 mg, 11.7 μmol, 18% yield) as a yellow solid.

### Example 293. Preparation of N—((S)-(4,4-difluorocyclohexyl)(3-((3,3-dimethylpyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 342)

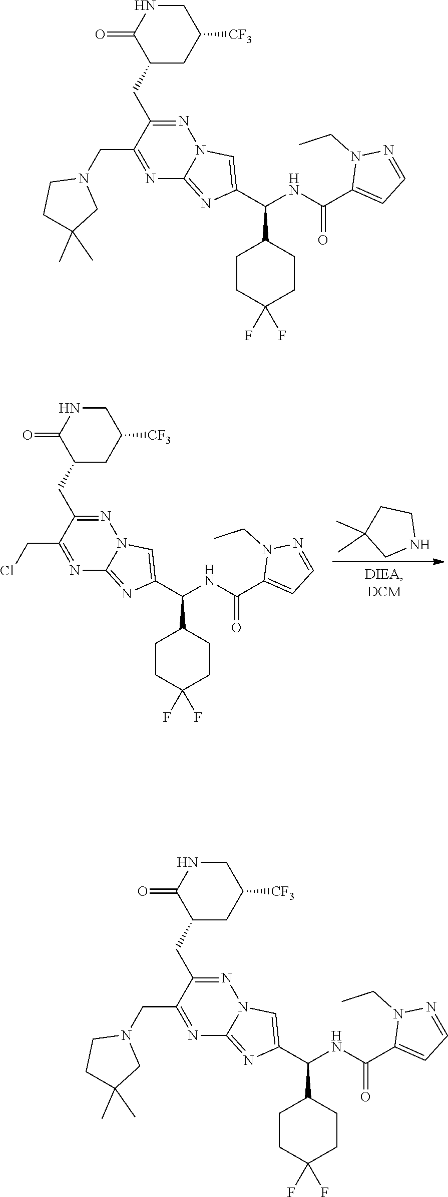

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 3,3-dimethylpyrrolidine hydrochloride (21.9 mg, 162 μmol, 2.00 eq, HCl salt). Purification by Prep-TLC (SiO₂, EtOAc/MeOH=20:1) afforded N—((S)-(4,4-difluorocyclohexyl)(3-((3,3-dimethylpyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (1.09 mg, 1.52 μmol, 1.9% yield) as a yellow solid.

### Example 294. Preparation of N-((1S)-(4,4-difluorocyclohexyl)(3-((2-methylpyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 343)

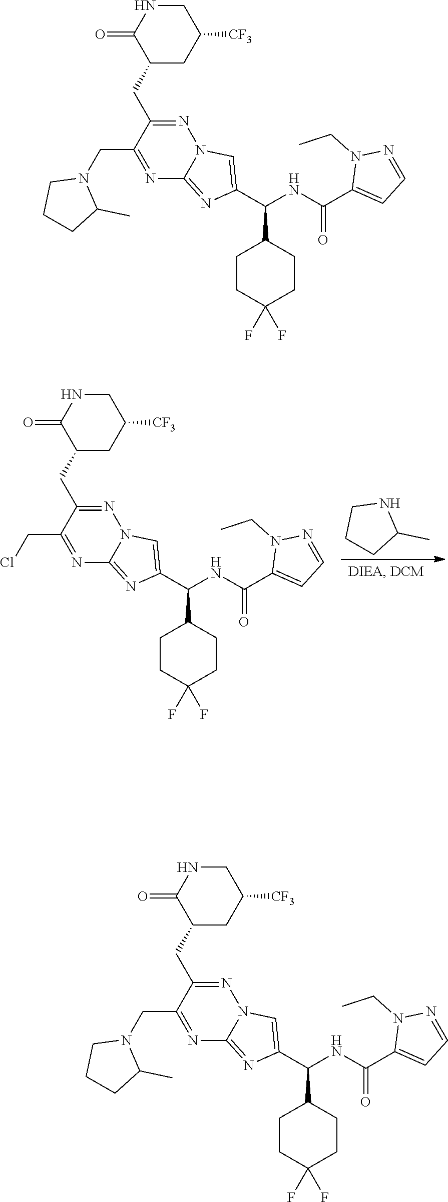

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 2-methylpyrrolidine hydrochloride (29.5 mg, 243 μmol, 3.00 eq, HCl salt). Purification by Prep-TLC (SiO₂, EtOAc/MeOH=20:1) then Prep-HPLC (Base condition: column: Waters Xbridge 150*25 mm*5 um; mobile phase: [water (ammonium hydroxide v/v)—ACN]; gradient: 44%-74% B over 9 min) afforded N-((1 S)-(4,4-difluorocyclohexyl)(3-((2-methylpyrrolidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (7.49 mg, 10.5 μmol, 13% yield) as a yellow solid.

### Example 295. Preparation of N—((S)-(3-((5-azaspiro[2.4]heptan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 344)

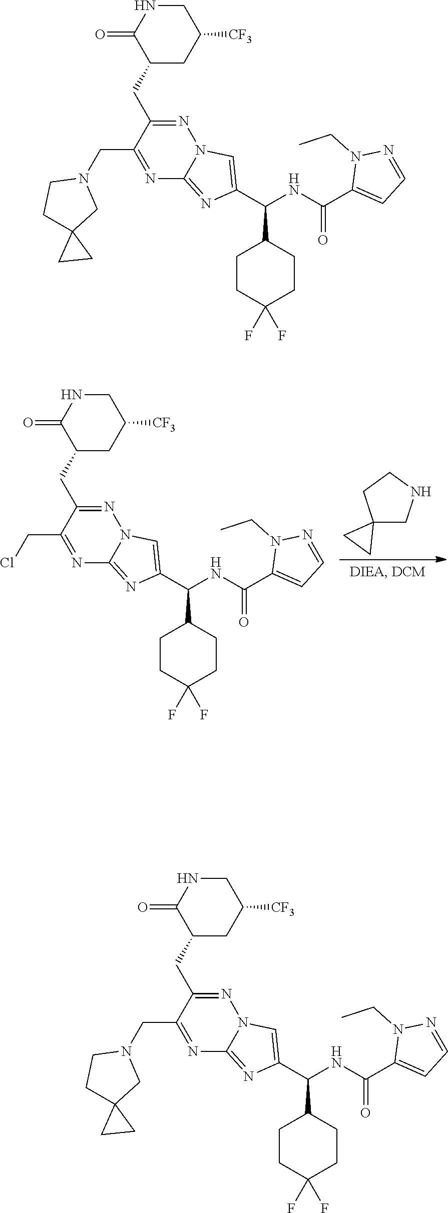

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (45.0 mg, 72.9 μmol, 1.00 eq) and 5-azaspiro[2.4]heptane hydrochloride (12.6 mg, 94.8 μmol, 1.30 eq, HCl salt). Purification by Prep-TLC (SiO₂, EtOAc) afforded N—((S)-(3-((5-azaspiro[2.4]heptan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (13.3 mg, 18.7 μmol, 26% yield) as a yellow solid.

### Example 296. Preparation of N—((S)-(3-((6-azaspiro[2.5]octan-6-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 346)

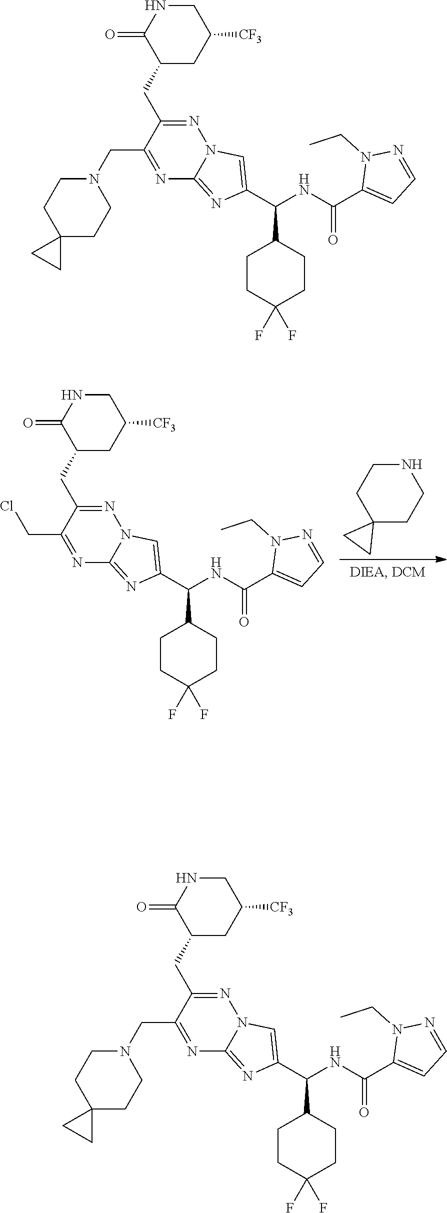

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (51l 1 mg, 82.9 μmol, 1.00 eq) and 6-azaspiro[2.5]octane (11.0 mg, 99.5 μmol, 1.20 eq). Purification by Prep-TLC (SiO₂, EtOAc/MeOH=20:1) then Prep-HPLC (column: Waters Xbridge 150*25 mm*5 um; mobile phase: [water (NH₄HCO₃)—ACN]; gradient: 46%-66% B over 10 min) afforded N—((S)-(3-((6-azaspiro[2.5]octan-6-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (29.62 mg, 42.1 μmol. 51% yield) as a white solid.

### Example 297. Preparation of N—((S)-(3-((5-azaspiro[2.5]octan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 347)

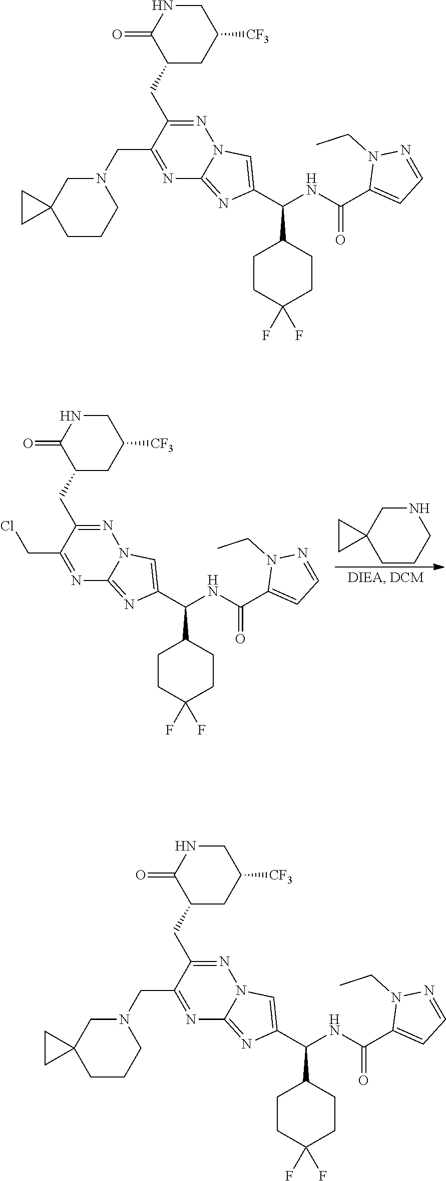

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (45.0 mg, 72.9 μmol, 1.00 eq) and 5-azaspiro[2.5]octane hydrochloride (16.1 mg, 109 μmol, 1.50 eq, HCl salt). Purification by Prep-TLC (SiO₂, EtOAc/MeOH=20:1) afforded N—((S)-(3-((5-azaspiro[2.5]octan-5-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (26.86 mg, 37.8 μmol, 52% yield) as a white solid.

### Example 298. Preparation of N-((1S)-(3-((6,6-difluoro-3-azabicyclo[3.1.1]heptan-3-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 348)

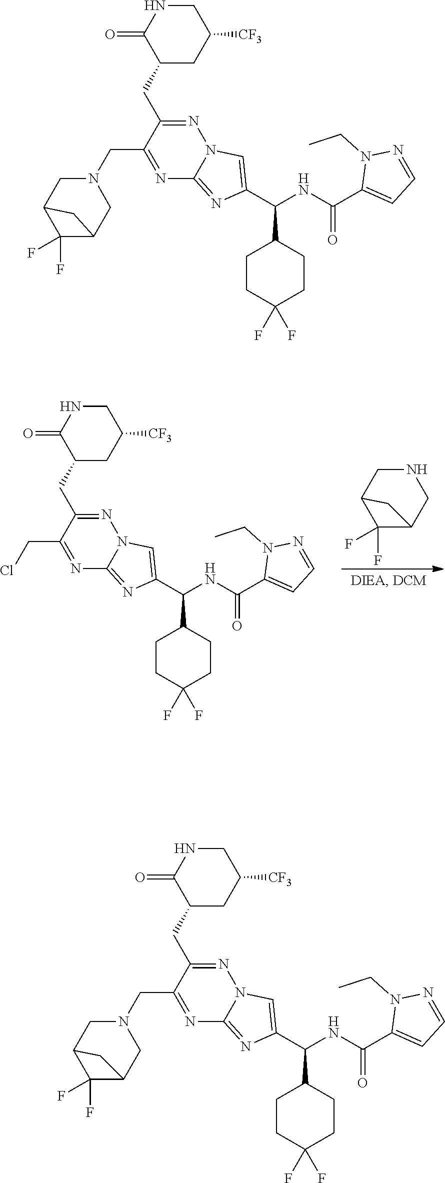

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 6,6-difluoro-3-azabicyclo[3.1.1]heptane (17.2 mg, 101 μmol, 1.57 eq, HCl salt). Purification by Prep-TLC (SiO₂, EtOAc) afforded N-((1S)-(3-((6,6-difluoro-3-azabicyclo[3.1.1]heptan-3-yl)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (25.42 mg, 34.3 μmol, 53% yield) as a white solid.

### Example 299. Preparation of N-((1S)-(4,4-difluorocyclohexyl)(3-((2-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 349)

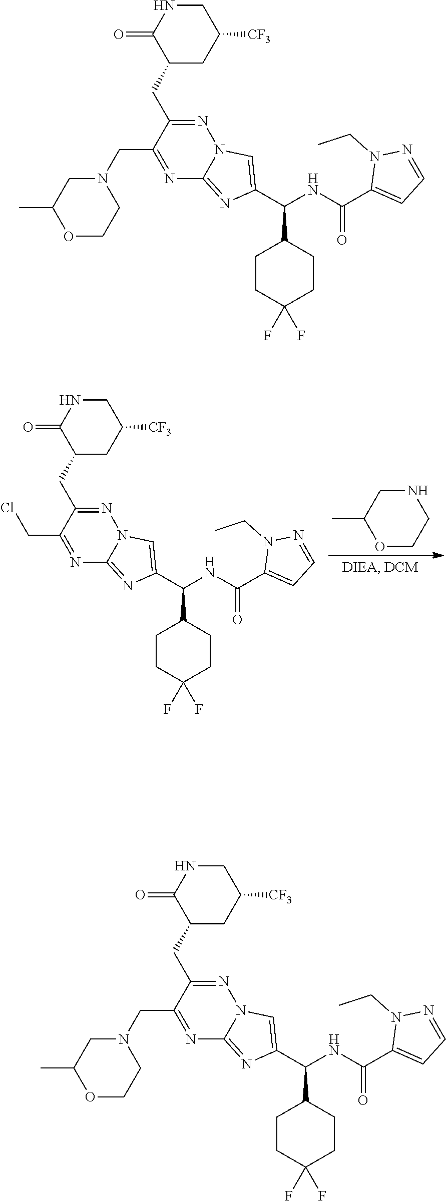

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (40.0 mg, 64.8 μmol, 1.00 eq) and 2-methylmorpholine (9.84 mg, 97.2 μmol, 1.50 eq). Purification by Prep-TLC (SiO₂, EtOAc/MeOH=20:1) afforded N-((1S)-(4,4-difluorocyclohexyl)(3-((2-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (31.26 mg, 44.7 μmol, 69% yield) as a yellow solid.

### Example 300. Preparation of N—((S)-(4,4-difluorocyclohexyl)(3-((3-oxo-4-(2,2,2-trifluoroethyl)piperazin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 352)

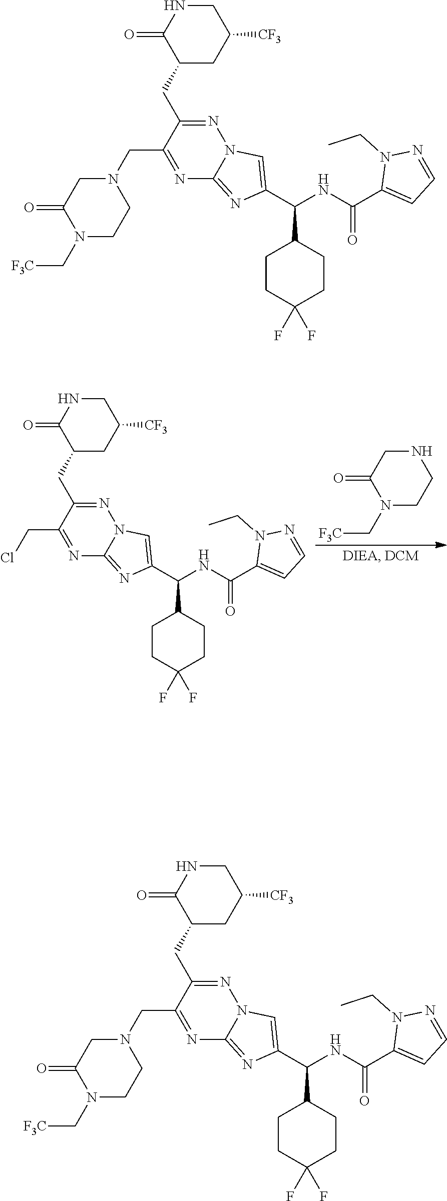

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 1-(2,2,2-trifluoroethyl)piperazin-2-one (29.5 mg, 162 μmol, 2.00 eq). Purification by Prep-TLC (SiO₂, EtOAc) afforded N—((S)-(4,4-difluorocyclohexyl)(3-((3-oxo-4-(2,2,2-trifluoroethyl)piperazin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (52.93 mg, 66.5 μmol, 82% yield) as a yellow solid.

### Example 301. Preparation of N—((S)-(3-((cyclopropyl(methyl)amino)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (Compound 356)

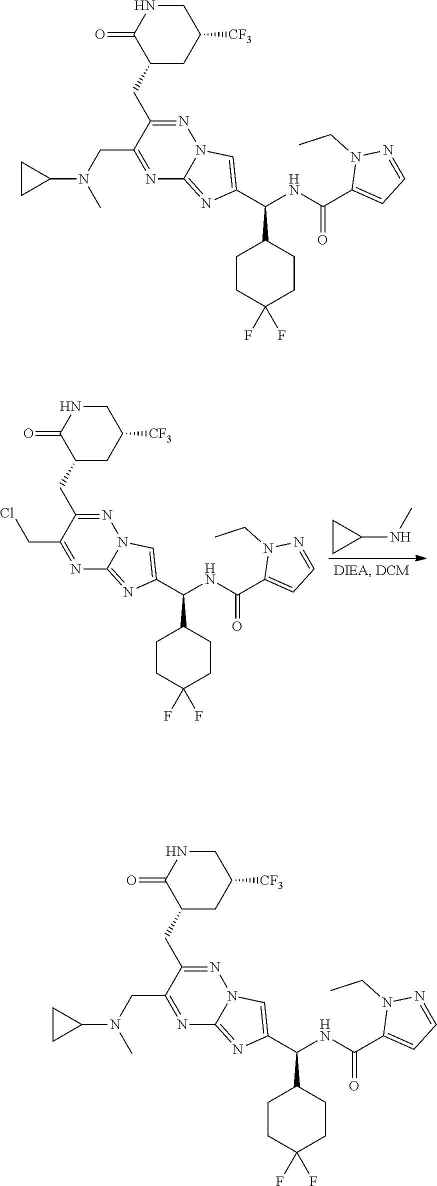

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (45.0 mg, 72.9 μmol, 1.00 eq) and N-methylcyclopropanamine (47.1 mg, 438 μmol, 6.00 eq, HCl salt). Purification by Prep-TLC (SiO₂, EtOAc) afforded N—((S)-(3-((cyclopropyl(methyl)amino)methyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (27.3 mg, 40.9 μmol, 56% yield) as a white solid.

### Example 302. Preparation of N—((S)-(4,4-difluorocyclohexyl)(3-((4-fluoropiperidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 371)

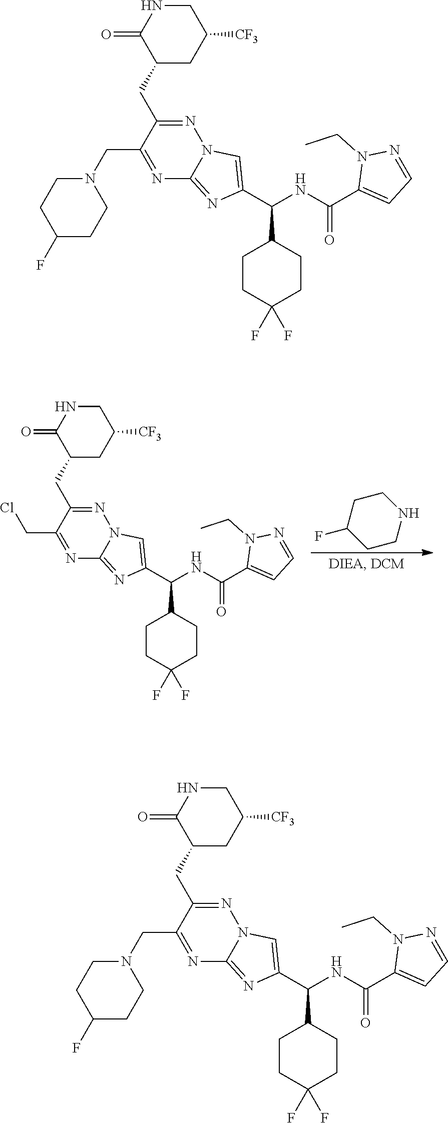

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 4-fluoropiperidine hydrochloride (16.9 mg, 121 μmol, 1.50 eq, HCl salt). Purification by prep-TLC (SiO₂, EtOAc/Pet Ether=5:1) afforded N—((S)-(4,4-difluorocyclohexyl)(3-((4-fluoropiperidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (28.74 mg, 40.99 μmol, 51% yield) as a yellow solid.

### Example 303. Preparation of 1-ethyl-N—((S)-((1r,4S)-4-methylcyclohexyl)(3-(morpholinomethyl)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (Compound 374)

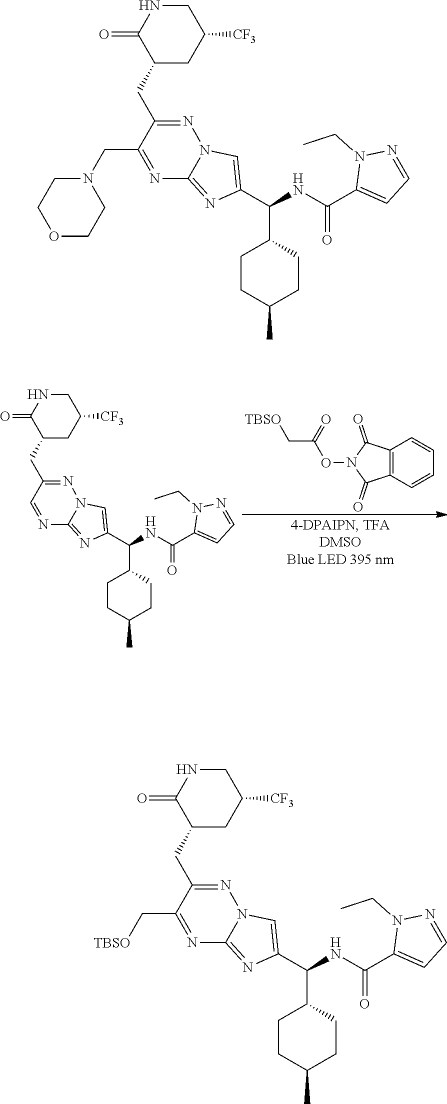

Intermediate N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide was synthesized following General Procedure 7 with 1-ethyl-N—((S)-((1r,4S)-4-methylcyclohexyl)(2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (1.40 g, 2.56 mmol, 1.00 eq) and 1,3-dioxoisoindolin-2-yl 2-((tert-butyldimethylsilyl)oxy)acetate (1.98 g, 5.89 mmol, 2.30 eq) and was purified by column chromatography (SiO₂, Pet. Ether/EtOAc=1:0 to 0:1) to afford N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (0.550 g, 796 μmol, 31% yield) as a yellow solid. LCMS [M+H]⁺=691.5 m/z.

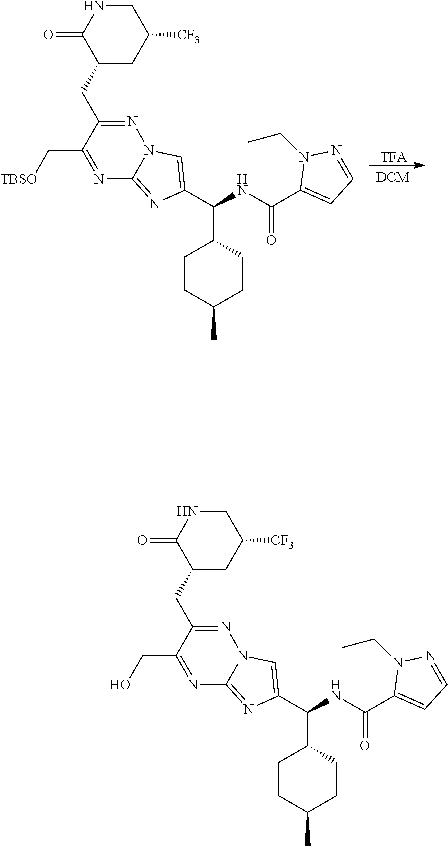 [SMILES: CCn1nccc1C(=O)N[C@H](c1cn2nc(C[C@H]3C[C@@H](C(F)(F)F)CNC3=O)c(CO[Si](C)(C)C(C)(C)C)nc2n1)[C@H]1CC[C@H](C)CC1 ; CCn1nccc1C(=O)N[C@H](c1cn2nc(C[C@H]3C[C@@H](C(F)(F)F)CNC3=O)c(CO)nc2n1)[C@H]1CC[C@H](C)CC1 ; "TFA"]

To a solution of N—((S)-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (0.500 g, 723 μmol, 1.00 eq) in DCM (5.00 mL) was added TFA (1.65 g, 14.4 mmol, 1.08 mL, 20.0 eq). The reaction mixture was stirred at 25° C. for 12 h then concentrated under reduced pressure to remove DCM and TFA. The pH was then adjusted to 8 with sat. aq. NaHCO₃ and the mixture was extracted with ethyl acetate 90.0 mL (30.0 mL*3). The combined organic layers were dried over Na₂SO₄, filtered, and concentrated under reduced pressure to give a residue which was purified by Prep-HPLC (FA condition; column: Phenomenex luna C 18 150*25 mm*10 um; mobile phase: [water (FA)—ACN]; gradient: 40%-70% B over 10 min) to afford 1-ethyl-N—((S)-(3-(hydroxymethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1H-pyrazole-5-carboxamide (330 mg, 572 μmol, 79% yield) as a yellow solid. LCMS [M+H]⁺=577.4 m/z.

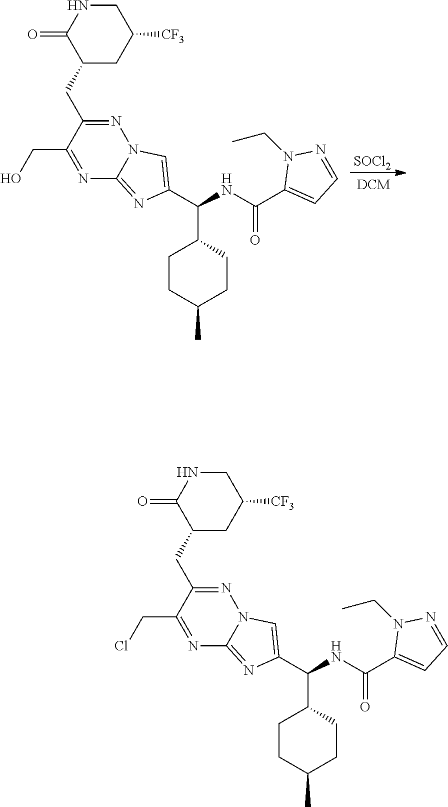

To a solution of 1-ethyl-N—((S)-(3-(hydroxymethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1H-pyrazole-5-carboxamide (150 mg, 260 μmol. 1.00 eq) in DCM (1.50 mL) was added SOCl₂ (154 mg, 1.30 mmol, 94.4 μL, 5.00 eq) at 0° C. The mixture was stirred at 25° C. for 1 h then concentrated under reduced pressure to remove SOCl₂ and DCM. Crude product, N—((S)-(3-(chloromethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (150 mg, crude), was obtained as a yellow solid and was used directly in the next step without further purification. LCMS [M+H]⁺=595.3 m/z.

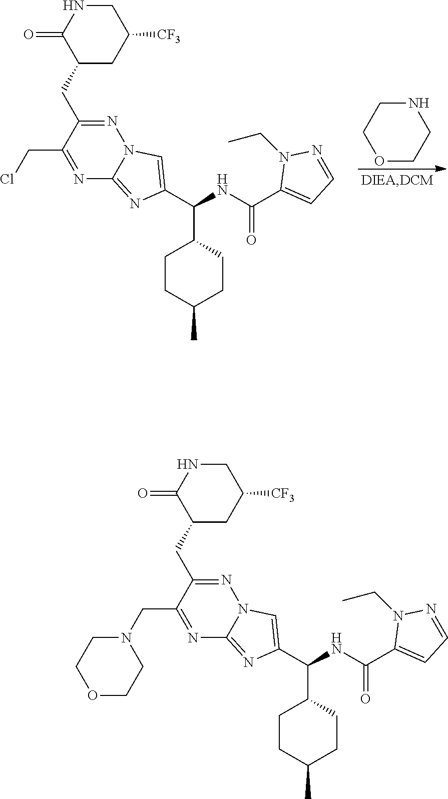 [SMILES: CCn1nccc1C(=O)N[C@H](c1cn2nc(C[C@H]3C[C@@H](C(F)(F)F)CNC3=O)c(CCl)nc2n1)[C@H]1CC[C@H](C)CC1 ; C1COCCN1 ; CCn1nccc1C(=O)N[C@H](c1cn2nc(C[C@H]3C[C@@H](C(F)(F)F)CNC3=O)c(CN3CCOCC3)nc2n1)[C@H]1CC[C@H](C)CC1]

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (50.0 mg, 84.0 μmol, 1.00 eq) and morpholine (14.6 mg, 168 μmol, 14.7 μL, 2.00 eq). Purification by Prep-HPLC (FA condition; column: Phenomenex luna C 18 150*25 mm*10 um; mobile phase: [water (FA)—ACN]; gradient: 21%-51% B over 10 min) afforded 1-ethyl-N—((S)-((1r,4S)-4-methylcyclohexyl)(3-(morpholinomethyl)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (20.84 mg, 31.6 μmol, 36% yield over 2 steps) as a yellow solid.

### Example 304. Preparation of N—((S)-(3-((3,3-difluoropyrrolidin-1-yl)methyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (Compound 375)

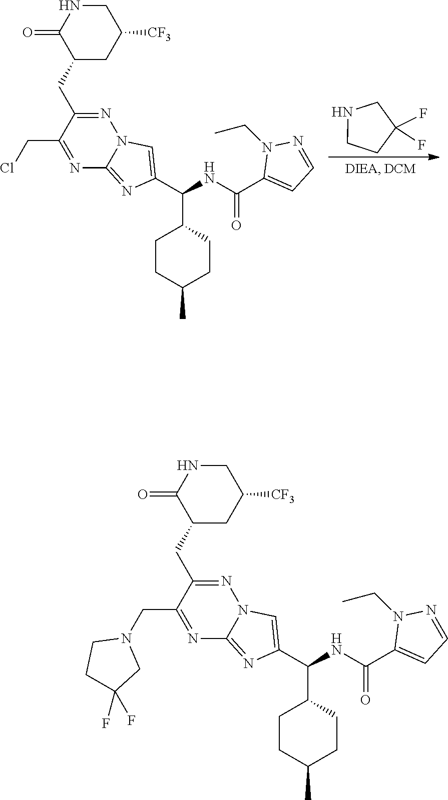

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (50.0 mg, 84.0 μmol, 1.00 eq) and 3,3-difluoropyrrolidine (18.0 mg, 168 μmol, 2.00 eq). Purification by Prep-HPLC (FA condition; column: Phenomenex luna C 18 150*25 mm*10 um; mobile phase: [water (FA)—ACN]; gradient: 44%-64% B over 10 min) then again by Prep-HPLC (neutral condition; column: Waters Xbridge 150*25 mm*5 um; mobile phase: [water (NH₄HCO₃)—ACN]; gradient: 40%-60% B over 10 min) afforded N—((S)-(3-((3,3-difluoropyrrolidin-1-yl)methyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (11.07 mg, 14.9 μmol, 17% yield over 2 steps) as a white solid.

### Example 305. Preparation of 1-ethyl-N-((1S)-((1r,4S)-4-methylcyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (Compound 408)

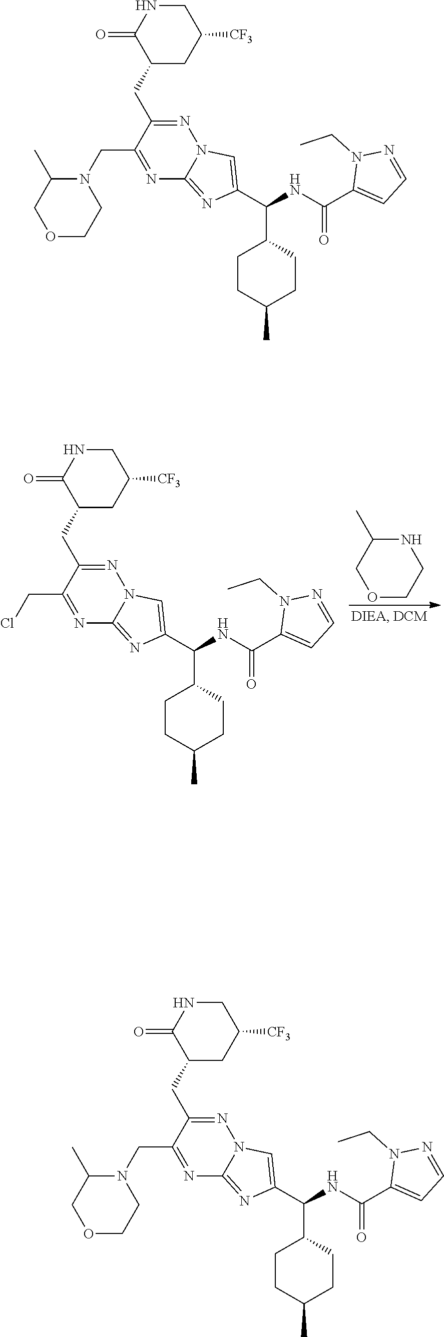

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)((1r,4S)-4-methylcyclohexyl)methyl)-1-ethyl-1H-pyrazole-5-carboxamide (50.0 mg, 84.0 μmol, 1.00 eq) and 3-methylmorpholine (17.0 mg, 168 μmol, 2.00 eq). Purification by Prep-HPLC (FA condition; column: Phenomenex luna C 18 150*25 mm*10 um; mobile phase: [water (FA)—ACN]; gradient: 23%-53% B over 10 min) afforded 1-ethyl-N-((1S)-((1r,4S)-4-methylcyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1H-pyrazole-5-carboxamide (18.36 mg, 26.9 μmol, 31% yield over 2 steps) as a yellow solid.

### Example 306. Preparation of N-((1S)-(4,4-difluorocyclohexyl)(3-(1-morpholinoethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 381 and 227)

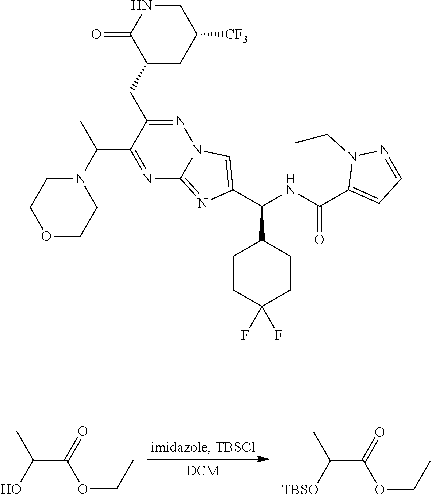

To a solution of ethyl 2-hydroxypropanoate (5.00 g, 42.3 mmol, 1.00 eq) in DMF (6.00 mL) was added TBSCl (7.02 g, 46.5 mmol, 5.73 mL, 1.10 eq) and imidazole (7.20 g, 105 mmol, 2.50 eq). The mixture was stirred at 25° C. for 3 h, then the reaction mixture was diluted with H₂O (20.0 mL) and extracted with EtOAc (50.0 mL*3). The combined organic layers were dried over Na₂SO₄, filtered, and concentrated under reduced pressure to give ethyl 2-((tert-butyldimethylsilyl)oxy)propanoate (8.50 g, 36.5 mmol, 86% yield) as a colorless oil. ¹H NMR (400 MHz, CDCl₃) δ 4.35-4.27 (m, 1H), 4.23-4.12 (m, 2H), 1.39 (d, J=6.8 Hz, 3H), 1.27 (t, J=7.2 Hz, 3H), 0.93-0.89 (m, 9H), 0.08 (d, J=12 Hz, 6H).

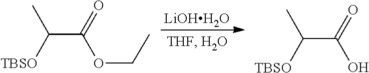

To a solution of ethyl 2-((tert-butyldimethylsilyl)oxy)propanoate (9.84 g, 42.3 mmol, 1.00 eq) in THF (50.0 mL) was added LiOH·H₂O (7.11 g, 169 mmol, 4.00 eq) in H₂O (10.0 mL) at 0° C. The mixture was stirred at 25° C. for 2 h, then the reaction mixture was quenched by addition of 1 M HCl (80.0 mL, pH≈6), diluted with H₂O (20.0 mL), extracted with EtOAc (100 mL*3). The combined organic layers were dried over Na₂SO₄, filtered, and concentrated under reduced pressure to give a residue. The product, 2-((tert-butyldimethylsilyl)oxy)propanoic acid (8.00 g, 39.1 mmol, 92% yield) was obtained as a colorless oil. ¹H NMR (400 MHz, CDCl₃) δ 4.40-4.21 (m, 1H), 1.50-1.35 (m, 3H), 0.95-0.91 (m, 9H), 0.15-0.07 (m, 6H).

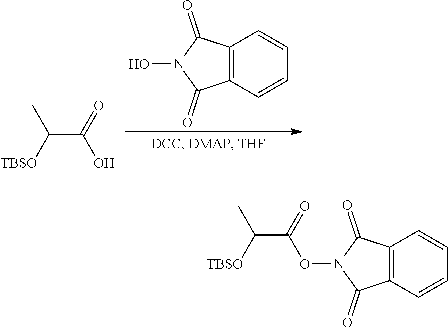 [SMILES: CC(O[Si](C)(C)C(C)(C)C)C(=O)O ; O=C1c2ccccc2C(=O)N1O ; CC(O[Si](C)(C)C(C)(C)C)C(=O)ON1C(=O)c2ccccc2C1=O]

To a solution of 2-((tert-butyldimethylsilyl)oxy)propanoic acid (3.00 g, 14.6 mmol, 1.00 eq) and 2-hydroxyisoindoline-1,3-dione (2.99 g, 18.3 mmol, 1.25 eq) in THF (10.0 mL) was added DCC (3.33 g, 16.1 mmol, 3.27 mL, 1.10 eq) and DMAP (179 mg, 1.47 mmol, 0.10 eq). The mixture was stirred at 25° C. for 5 h. The reaction mixture was filtered with EtOAc washes (30.0 mL) and the filtrate was concentrated under reduced pressure to give a residue. The residue was purified by column chromatography (SiO₂, Pet. Ether/EtOAc=20:1 to 5:1) to afford 1,3-dioxoisoindolin-2-yl 2-((tert-butyldimethylsilyl)oxy)propanoate (2.60 g, 7.44 mmol, 51% yield) as a colorless oil. ¹H NMR (400 MHz, CDCl₃) δ 7.90-7.76 (m, 4H), 4.75-4.70 (m, 1H), 1.63 (d, J=6.8 Hz, 3H), 0.96-0.92 (m, 9H), 0.18-0.15 (m, 6H).

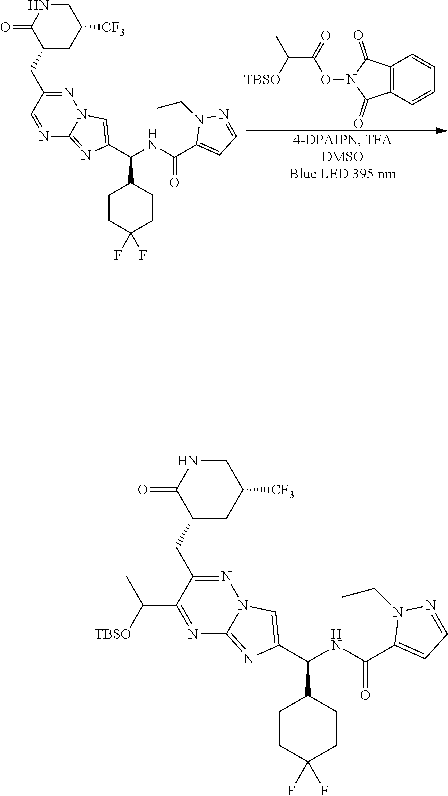 [SMILES: CCn1nccc1C(=O)N[C@H](c1cn2nc(C[C@H]3C[C@@H](C(F)(F)F)CNC3=O)cnc2n1)C1CCC(F)(F)CC1 ; CC(O[Si](C)(C)C(C)(C)C)C(=O)ON1C(=O)c2ccccc2C1=O ; "4-DPAIPN, TFADMSOBlue LED 395 nm" ; CCn1nccc1C(=O)N[C@H](c1cn2nc(C[C@H]3C[C@@H](C(F)(F)F)CNC3=O)c(C(C)O[Si](C)(C)C(C)(C)C)nc2n1)C1CCC(F)(F)CC1]

Intermediate N-((1 S)-(3-(1-((tert-butyldimethylsilyl)oxy)ethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide was synthesized following General Procedure 7 with N—((S)-(4,4-difluorocyclohexyl)(2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (0.20 g, 351 μmol, 1.00 eq) and 1,3-dioxoisoindolin-2-yl 2-((tert-butyldimethylsilyl)oxy)propanoate (245 mg, 703 μmol, 2.00 eq) and was purified by prep-HPLC (column: Phenomenex luna C 18 150*25 mm*10 um; mobile phase: [water (FA)—ACN]; gradient: 66%-86% B over 10 mins) to afford N-((1S)-(3-(1-((tert-butyldimethylsilyl)oxy)ethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (70.0 mg, 92.4 μmol, 27% yield) as a white solid. LCMS [M+H]⁺=727.4 m/z.

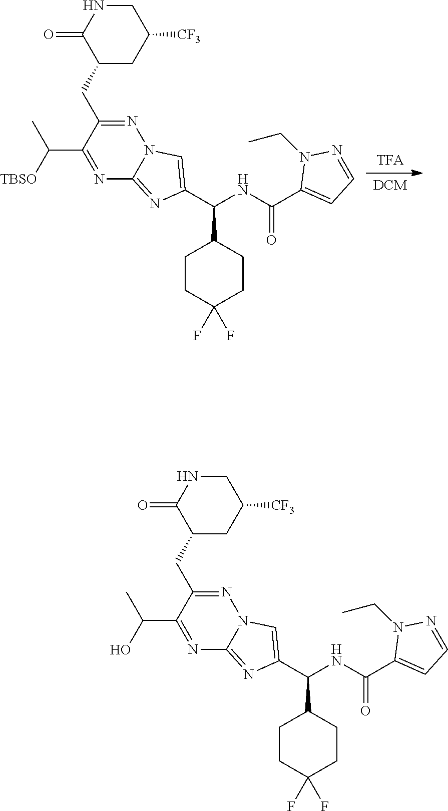 [SMILES: CCn1nccc1C(=O)N[C@H](c1cn2nc(C[C@H]3C[C@@H](C(F)(F)F)CNC3=O)c(C(C)O[Si](C)(C)C(C)(C)C)nc2n1)C1CCC(F)(F)CC1 ; CCn1nccc1C(=O)N[C@H](c1cn2nc(C[C@H]3C[C@@H](C(F)(F)F)CNC3=O)c(C(C)O)nc2n1)C1CCC(F)(F)CC1]

To a solution N-((1S)-(3-(1-((tert-butyldimethylsilyl)oxy)ethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (110 mg, 151 μmol, 1.00 eq) in DCM (2.00 mL) was added TFA (345 mg, 3.03 mmol, 224 μL, 20.0 eq). The mixture was stirred at 0° C. for 2 h. The reaction mixture was quenched with sat. aq. NaHCO₃ (50.0 mL, pH~8) and extracted with EtOAc (30.0 mL*3). The combined organic layers were dried over Na₂SO₄, filtered, and concentrated under reduced pressure to give a residue. The residue was purified by prep-TLC (EtOAc/MeOH=20:1). The product, N-((1S)-(4,4-difluorocyclohexyl)(3-(1-hydroxyethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (75.0 mg, 121 μmol, 80% yield) was obtained as a white solid. LCMS [M+H]⁺=613.3 m/z.

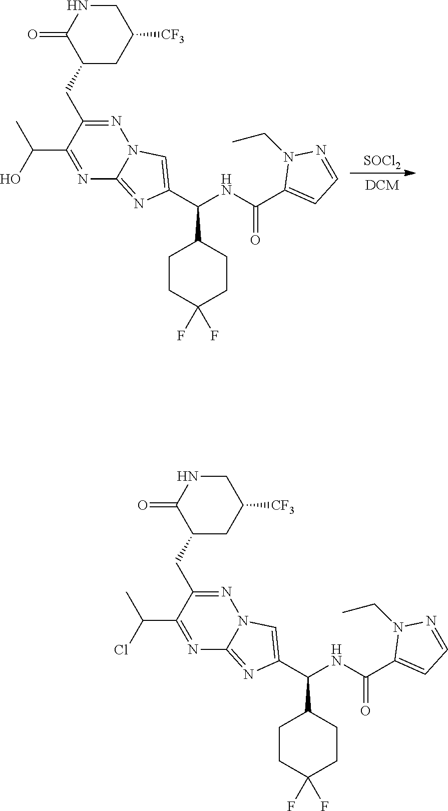

To a solution of N-((1S)-(4,4-difluorocyclohexyl)(3-(1-hydroxyethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (75.0 mg, 122 μmol, 1.00 eq) in DCM (2.00 mL) was added SOCl₂ (29.1 mg, 244 μmol, 17.7 μL, 2.00 eq) at 0° C. The mixture was stirred at 0° C. for 1 h then concentrated under reduced pressure to give a residue. Crude product, N-((1 S)-(3-(1-chloroethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (70.0 mg, 110 μmol, 91% yield), was obtained as a yellow solid. LCMS [M+H]⁺=631.3 m/z.

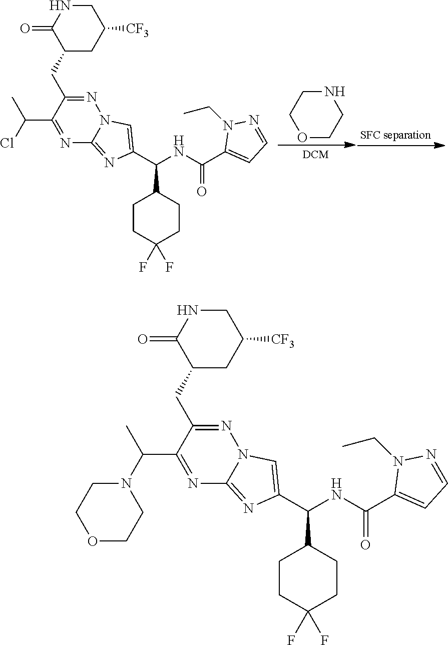

Nucleophilic substitution was carried out following General Procedure 15 using N-((1S)-(3-(1-chloroethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (60.0 mg, 95.0 μmol, 1.00 eq) and morpholine (60.0 mg, 95.0 μmol, 1.00 eq). Purification by . . . afforded a mixture of stereoisomers of N-((1S)-(4,4-difluorocyclohexyl)(3-(1-morpholinoethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (50.0 mg, 73.3 μmol, 77% yield) as a white solid. LCMS [M+H]⁺=682.4 m/z.

The residue was purified by prep-SFC (column: DAICEL CHIRALPAK IG (250 mm*30 mm, 10 um); mobile phase: [CO₂—ACN/i-PrOH (0.1% NH₃H₂O)]; B %: 55%, isocratic elution mode, Rₜ=1.233, 1.779).

The first eluting single stereoisomer of N-((1S)-(4,4-difluorocyclohexyl)(3-(1-morpholinoethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 381 20.0 mg, 28.6 μmol, 39% yield) was obtained as a white solid.

The second eluting single stereoisomer of N-((l S)-(4,4-difluorocyclohexyl)(3-(1-morpholinoethyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 227 9.97 mg, 14.6 μmol, 1.00 eq) was obtained as a white solid.

### Example 307. Preparation of N—((S)-(4,4-difluorocyclohexyl)(3-((4-methylpiperazin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compounds 412)

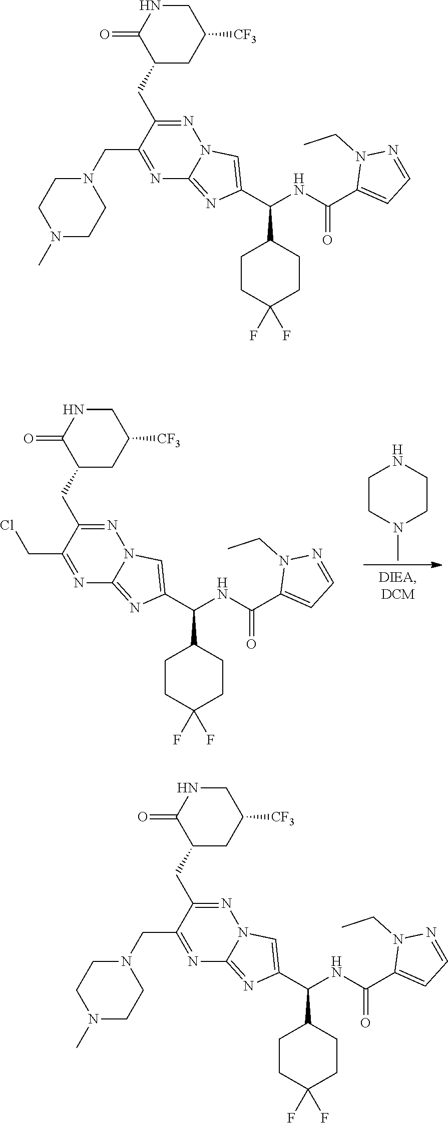

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (50.0 mg, 81.0 μmol, 1.00 eq) and 1-methylpiperazine (16.2 mg, 162 μmol, 17.9 μL, 2.00 eq). Purification by Prep-TLC (SiO₂, DCM/MeOH=10:1 afforded N—((S)-(4,4-difluorocyclohexyl)(3-((4-methylpiperazin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (44.29 mg, 62.7 μmol, 77% yield) as a light-yellow solid.

### Example 308. Preparation of N—((S)-2,2-dicyclopropyl-1-(3-(morpholinomethyl)-2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (Compound 406)

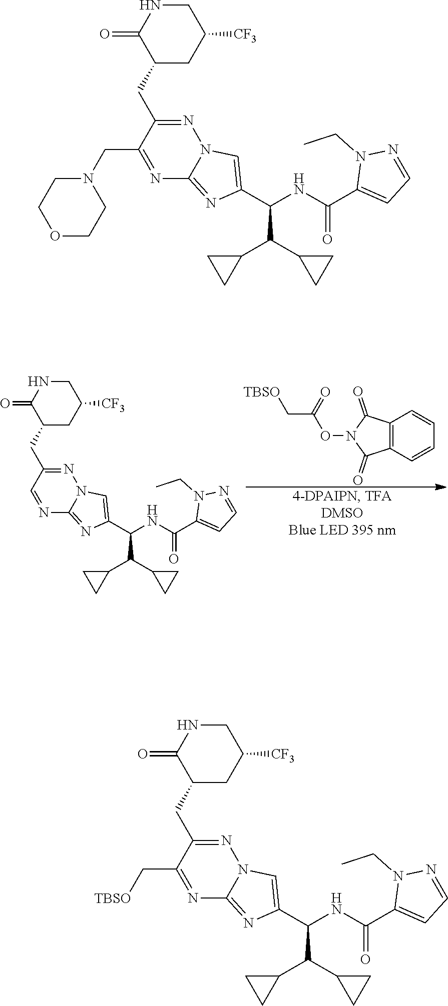

Intermediate N—((S)-1-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)-2,2-dicyclopropylethyl)-1-ethyl-1H-pyrazole-5-carboxamide was synthesized following General Procedure 7 with N—((S)-2,2-dicyclopropyl-1-(2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide 0.580 g, 1.07 mmol, 1.00 eq) and 1,3-dioxoisoindolin-2-yl 2-((tert-butyldimethylsilyl)oxy)acetate (2.00 eq) and was purified by prep-HPLC (FA condition, column: Phenomenex luna C18 150*25 mm*10 um; mobile phase: [water (FA)—ACN]; gradient: 69%-89% B over 10 min) to afford N—((S)-1-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)-2,2-dicyclopropylethyl)-1-ethyl-1H-pyrazole-5-carboxamide (0.500 g, 725 μmol, 68% yield) as a white solid. LCMS [M+H]⁺=689.3 nm/z.

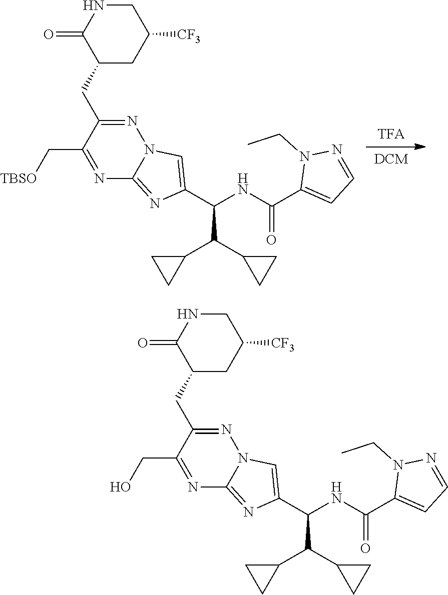

To a solution of N—((S)-1-(3-(((tert-butyldimethylsilyl)oxy)methyl)-2-(((3R,5R)-2-oxo-5-(trifluoromethyl)piperidin-3-yl)methyl)imidazo[1,2-b][1,2,4]triazin-6-yl)-2,2-dicyclopropylethyl)-1-ethyl-1H-pyrazole-5-carboxamide (120 mg, 174 μmol, 1.00 eq) in DCM (1.00 mL) was added TFA (397 mg, 3.48 mmol, 258 μL, 20.0 eq) at 0° C. The mixture was stirred at 25° C. for 3 h. The reaction mixture was diluted with H₂O (10.0 mL) and sat. aq. NaHCO₃ to adjust pH to 9. The mixture was extracted with DCM (20.0 mL*2). The combined organic layers were dried over Na₂SO₄, filtered, and concentrated under reduced pressure to give a residue which was then purified by Prep-TLC (SiO₂, EtOAc/MeOH=20:1) to afford N—((S)-2,2-dicyclopropyl-1-(3-(hydroxymethyl)-2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (40.0 mg, 69.6 μmol, 40% yield) as a white solid. LCMS [M+H]⁺=575.2 m/z.

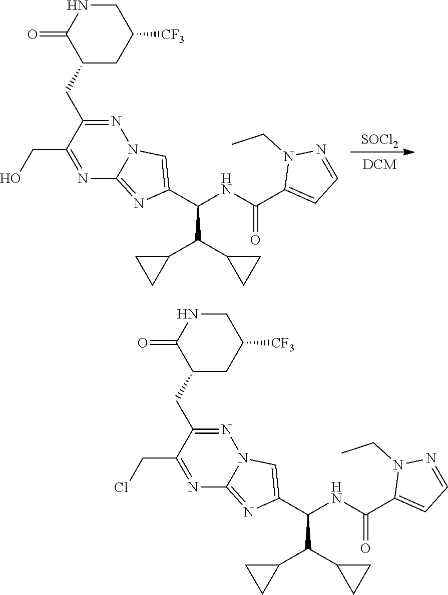

To a solution of N—((S)-2,2-dicyclopropyl-1-(3-(hydroxymethyl)-2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (40.0 mg, 69.6 μmol, 1.00 eq) in DCM (1.00 mL) was added SOCl₂ (16.5 mg, 139 μmol, 10.1 μL, 2.00 eq) at 0° C. under N₂. The mixture was stirred at 0° C. for 1 h, then concentrated under reduced pressure to give a residue. Crude product, N—((S)-1-(3-(chloromethyl)-2-(((3R,5R)-2-oxo-5-(trifluoromethyl)piperidin-3-yl)methyl)imidazo[1,2-b][1,2,4]triazin-6-yl)-2,2-dicyclopropylethyl)-1-ethyl-1H-pyrazole-5-carboxamide (0.410 mg, 69.1 μmol, 93% yield), was obtained as a white solid. LCMS [M+H]⁺=593.2 m/z.

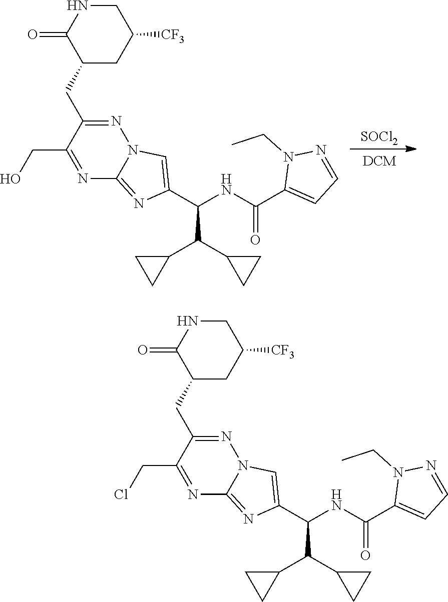

Nucleophilic substitution was carried out following General Procedure 15 using N—((S)-1-(3-(chloromethyl)-2-(((3R,5R)-2-oxo-5-(trifluoromethyl)piperidin-3-yl)methyl)imidazo[1,2-b][1,2,4]triazin-6-yl)-2,2-dicyclopropylethyl)-1-ethyl-1H-pyrazole-5-carboxamide (40.0 mg, 67.4 μmol, 1.00 eq) and morpholine (8.81 mg, 101 μmol, 8.90 μL, 1.50 eq). Purification by Prep-TLC (SiO₂, DCM/MeOH=10:1) afforded N—((S)-2,2-dicyclopropyl-1-(3-(morpholinomethyl)-2-(((3R,5R)-2-oxo-5-(trifluoromethyl)piperidin-3-yl)methyl)imidazo[1,2-b][1,2,4]triazin-6-yl)ethyl)-1-ethyl-1H-pyrazole-5-carboxamide (21.2 mg, 32.9 μmol, 49% yield) as a white solid.

### Example 309. Preparation of N—((S)-(4,4-difluorocyclohexyl)(3-((4,4-difluoropiperidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 345)

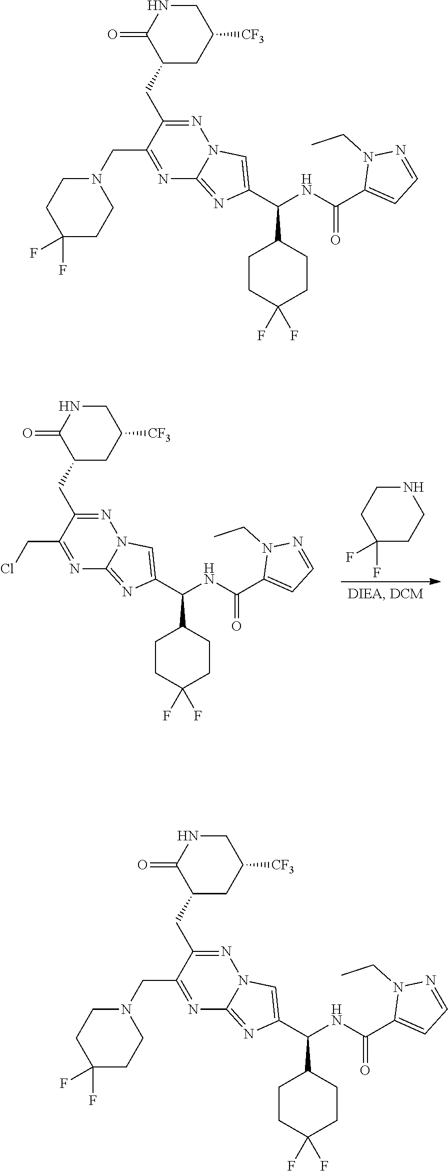

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (35.0 mg, 56.7 μmol, 1.00 eq) and 4,4-difluoropiperidine (6.87 mg, 56.7 μmol, 1.00 eq). Purification by Prep-TLC (SiO₂, Pet. Ether/EtOAc=1.1) afforded N—((S)-(4,4-difluorocyclohexyl)(3-((4,4-difluoropiperidin-1-yl)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (25.0 mg, 34.2 μmol, 60% yield) as a white solid.

### Example 310. Preparation of N—((S)-(4,4-difluorocyclohexyl)(3-((2,2-dimethylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 351)

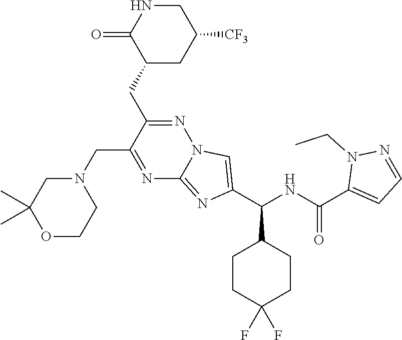 [SMILES: CCn1nccc1C(=O)N[C@H](c1cn2nc(C[C@H]3C[C@@H](C(F)(F)F)CNC3=O)c(CN3CCOC(C)(C)C3)nc2n1)C1CCC(F)(F)CC1]

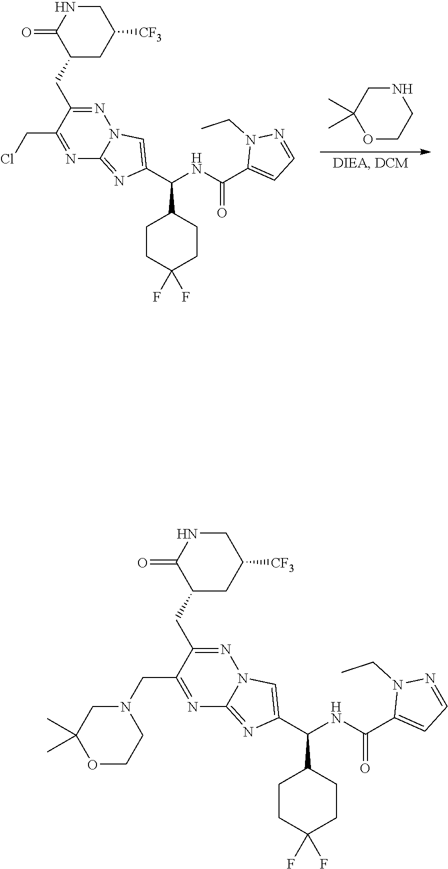

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (35.0 mg, 56.7 μmol, 1.00 eq) and 2,2-dimethylmorpholine (13.1 mg, 113 μmol, 2.00 eq). Purification by Prep-TLC (SiO₂, EtOAc) afforded N—((S)-(4,4-difluorocyclohexyl)(3-((2,2-dimethylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (24.94 mg, 34.4 μmol, 61% yield) as a light-yellow solid.

### Example 311. Preparation of N-((1S)-(4,4-difluorocyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 231 and 232)

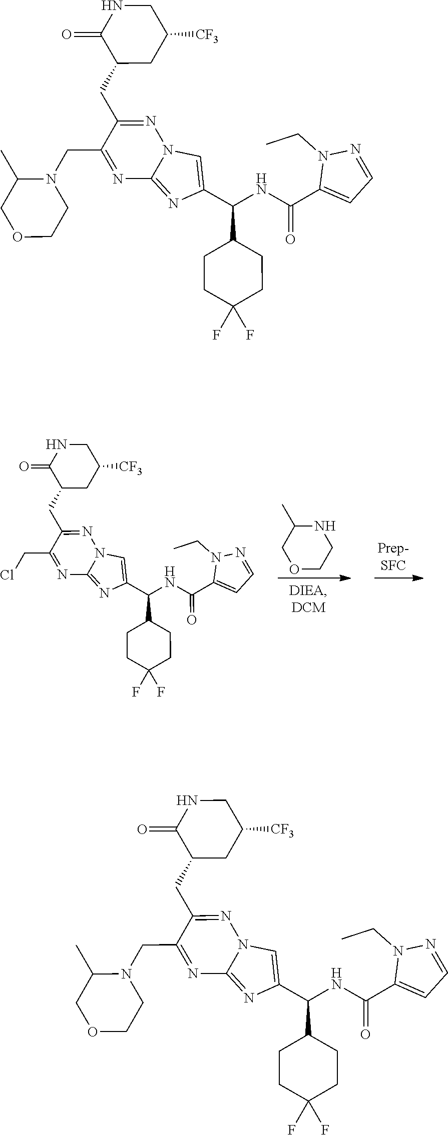

Nucleophilic substitution was carried out following General Procedure 15 using N—((S)-(3-(chloromethyl)-2-(((3R,5R)-2-oxo-5-(trifluoromethyl)piperidin-3-yl)methyl)imidazo[1,2-b][1,2,4]triazin-6-yl)(4,4-difluorocyclohexyl)methyl)-1-ethyl-1H-pyrazole-5-carboxamide (35.0 mg, 56.7 μmol, 1.00 eq) and 3-methylmorpholine (11.5 mg, 113 μmol, 2.00 eq). Purification by Prep-TLC (SiO₂, EtOAc) afforded N-((1S)-(4,4-difluorocyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (23.63 mg, 33.3 μmol, 59% yield) as a light-yellow solid.

The residue was purified by Prep-SFC (column: DAICEL CHIRALPAK IG (250 mm*30 mm, 10 um); mobile phase: [CO₂-EtOH]; B %:60%, isocratic elution mode), and the resulting solution was adjusted to pH=4 with 1 M HCl, then concentrated under the vacuum to afford Peaks 1 and 2. Peak 1 was further purified with Prep-HPLC (column: Phenomenex luna C18 150*25 mm*10 um; mobile phase: [water (FA)—ACN]; gradient: 20%-40% B over 10 mins), Peak 2 was further purified with Prep-HPLC (column: Phenomenex luna C18 150*25 mm*10 um; mobile phase: [water (FA)—ACN]; gradient: 20%-40% B over 10 mins).

The first eluting single stereoisomer of N-((l S)-(4,4-difluorocyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 231 6.37 mg, 8.72 μmol, 30% yield) was obtained as an off-white solid.

The second eluting single stereoisomer of N-((1 S)-(4,4-difluorocyclohexyl)(3-((3-methylmorpholino)methyl)-2-(((3R,5R)-2-oxo-5-(trifluoromethyl)piperidin-3-yl)methyl)imidazo[1,2-b][1,2,4]triazin-6-yl)methyl)-1-ethyl-1H-pyrazole-5-carboxamide (Compound 232 4.98 mg, 7.24 μmol, 25% yield) was obtained as a white solid.

### Example 312. Preparation of N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-2,2-difluoropropanamide (Compound 438)

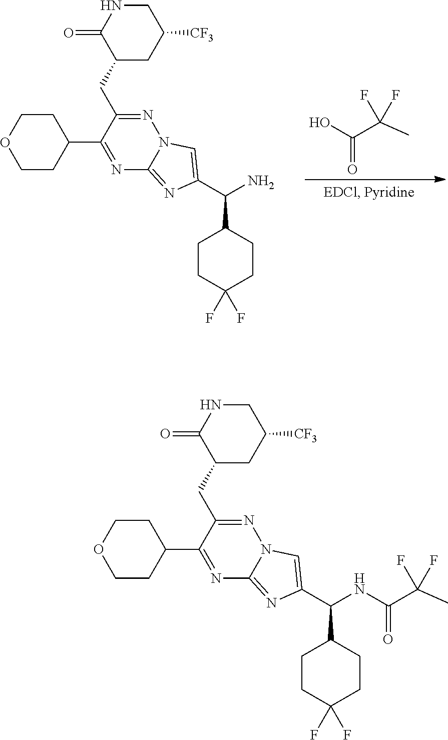

N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-2,2-difluoropropanamide was synthesized following the General Procedure 6 with (3R,5R)-3-((6-((S)-amino(4,4-difluorocyclohexyl)methyl)-3-(tetrahydro-2H-pyran-4-yl)imidazo[1,2-b][1,2,4]triazin-2-yl)methyl)-5-(trifluoromethyl)piperidin-2-one (100 mg, 188 μmol, 1.00 eq), 2,2-difluoropropanoic acid (103 mg, 942 μmol, 5.00 eq) and was purified by Prep-HPLC (column: Phenomenex luna C18 150 mm×25 mm, 10 um; mobile phase: [water (FA)—ACN]; gradient: 42%-62% B over 10 min) to afford N—((S)-(4,4-difluorocyclohexyl)(2-(((3R,5R)-2-oxo-5-(trifluoromethyl)piperidin-3-yl)methyl)-3-(tetrahydro-2H-pyran-4-yl)imidazo[1,2-b][1,2,4]triazin-6-yl)methyl)-2,2-difluoropropanamide (42.11 mg, 66.6 μmol, 35.3% yield, 98.5% purity) as a white solid.

### Example 313: Synthesis of Compounds 1-439

The compounds set forth in Table 1 were prepared using the synthetic procedures of Examples 1 to 312, or analogous procedures thereof. The compounds or salts in Table 1 represent single isolated isomers whose absolute stereochemistry may be unknown. Table 1 provides reported LCMS values, m/z as [M+H]⁺, for compounds 1-439.

TABLE 1
Structure and Spectroscopic Data for Compounds 1-439
Structure 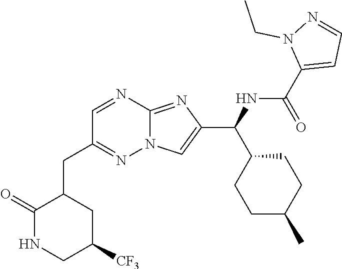 [SMILES: CCn1nccc1C(=O)N[C@H](c1cn2nc(CC3C[C@@H](C(F)(F)F)CNC3=O)cnc2n1)[C@H]1CC[C@H](C)CC1]
1 m/z = 547.3 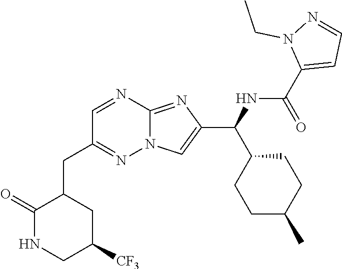 [SMILES: CCn1nccc1C(=O)N[C@H](c1cn2nc(CC3C[C@@H](C(F)(F)F)CNC3=O)cnc2n1)[C@H]1CC[C@H](C)CC1]
2 m/z = 547.3 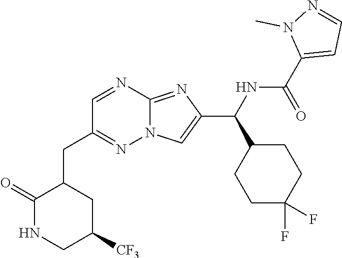 [SMILES: Cn1nccc1C(=O)N[C@H](c1cn2nc(CC3C[C@@H](C(F)(F)F)CNC3=O)cnc2n1)C1CCC(F)(F)CC1]
3 m/z = 555.1 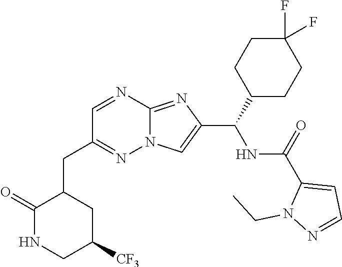 [SMILES: CCn1nccc1C(=O)N[C@H](c1cn2nc(CC3C[C@@H](C(F)(F)F)CNC3=O)cnc2n1)C1CCC(F)(F)CC1]
4 m/z = 569.2 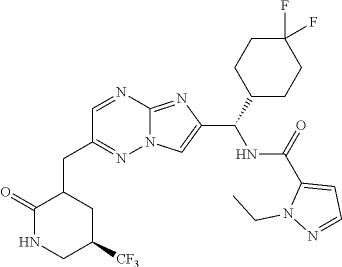 [SMILES: CCn1nccc1C(=O)N[C@H](c1cn2nc(CC3C[C@@H](C(F)(F)F)CNC3=O)cnc2n1)C1CCC(F)(F)CC1]
5 m/z = 569.2 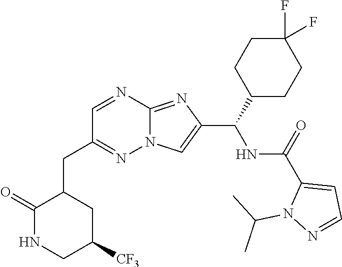 [SMILES: CC(C)n1nccc1C(=O)N[C@H](c1cn2nc(CC3C[C@@H](C(F)(F)F)CNC3=O)cnc2n1)C1CCC(F)(F)CC1]
6 m/z = 583.3 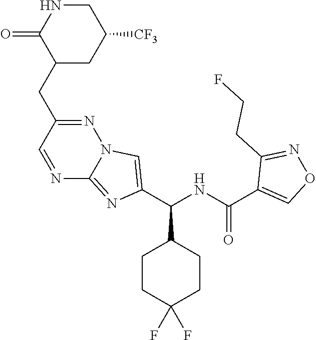 [SMILES: O=C(N[C@H](c1cn2nc(CC3C[C@@H](C(F)(F)F)CNC3=O)cnc2n1)C1CCC(F)(F)CC1)c1conc1CCF]
7 m/z = 588.3 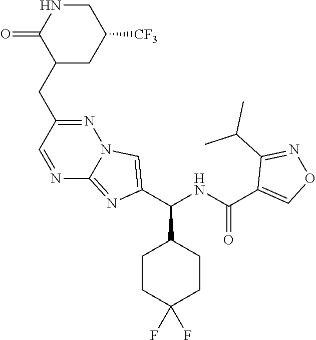 [SMILES: CC(C)c1nocc1C(=O)N[C@H](c1cn2nc(CC3C[C@@H](C(F)(F)F)CNC3=O)cnc2n1)C1CCC(F)(F)CC1]
8 m/z = 584.3 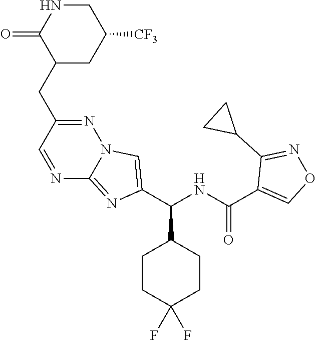 [SMILES: O=C(N[C@H](c1cn2nc(CC3C[C@@H](C(F)(F)F)CNC3=O)cnc2n1)C1CCC(F)(F)CC1)c1conc1C1CC1]
9 m/z = 582.3 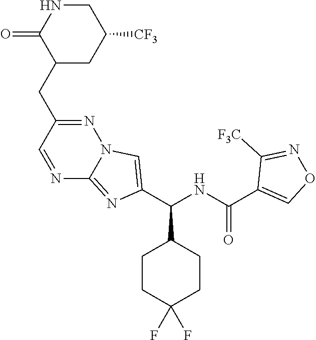 [SMILES: O=C(N[C@H](c1cn2nc(CC3C[C@@H](C(F)(F)F)CNC3=O)cnc2n1)C1CCC(F)(F)CC1)c1conc1C(F)(F)F]
10 m/z = 610.1 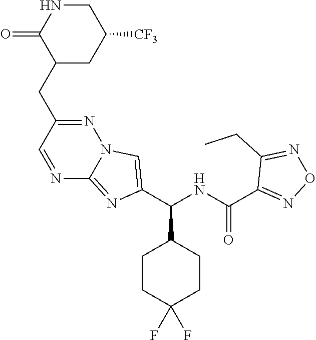 [SMILES: CCc1nonc1C(=O)N[C@H](c1cn2nc(CC3C[C@@H](C(F)(F)F)CNC3=O)cnc2n1)C1CCC(F)(F)CC1]
11 m/z = 571.3 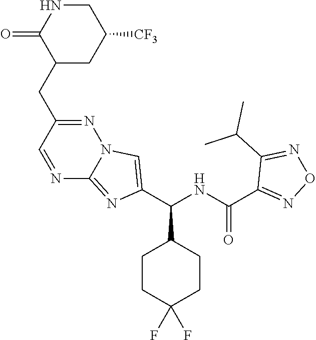 [SMILES: CC(C)c1nonc1C(=O)N[C@H](c1cn2nc(CC3C[C@@H](C(F)(F)F)CNC3=O)cnc2n1)C1CCC(F)(F)CC1]
12 m/z = 585.2 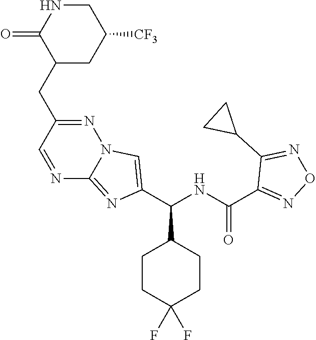 [SMILES: O=C(N[C@H](c1cn2nc(CC3C[C@@H](C(F)(F)F)CNC3=O)cnc2n1)C1CCC(F)(F)CC1)c1nonc1C1CC1]
13 m/z = 583.2 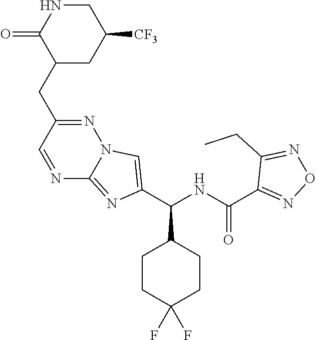 [SMILES: CCc1nonc1C(=O)N[C@H](c1cn2nc(CC3C[C@H](C(F)(F)F)CNC3=O)cnc2n1)C1CCC(F)(F)CC1]
14 m/z = 569.4 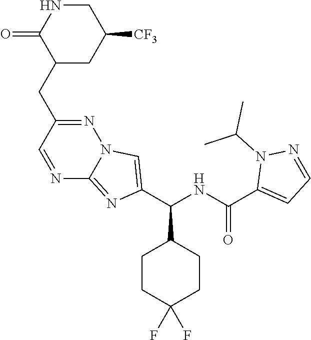 [SMILES: CC(C)n1nccc1C(=O)N[C@H](c1cn2nc(CC3C[C@H](C(F)(F)F)CNC3=O)cnc2n1)C1CCC(F)(F)CC1]
15 m/z = 583.4 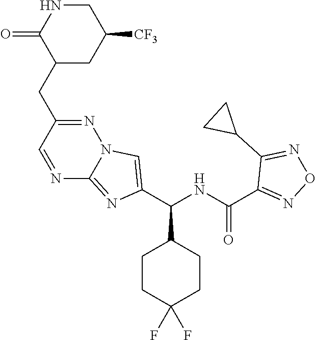 [SMILES: O=C(N[C@H](c1cn2nc(CC3C[C@H](C(F)(F)F)CNC3=O)cnc2n1)C1CCC(F)(F)CC1)c1nonc1C1CC1]
16 m/z = 583.4 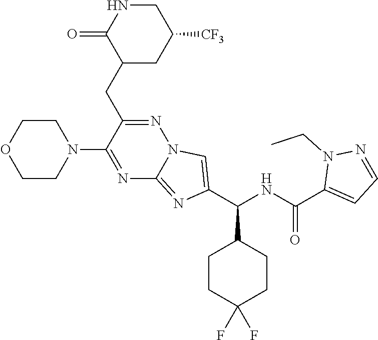 [SMILES: CCn1nccc1C(=O)N[C@H](c1cn2nc(CC3C[C@@H](C(F)(F)F)CNC3=O)c(N3CCOCC3)nc2n1)C1CCC(F)(F)CC1]
17 m/z = 654.4 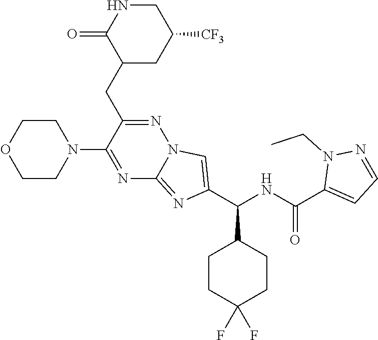 [SMILES: CCn1nccc1C(=O)N[C@H](c1cn2nc(CC3C[C@@H](C(F)(F)F)CNC3=O)c(N3CCOCC3)nc2n1)C1CCC(F)(F)CC1]
18 m/z = 654.4 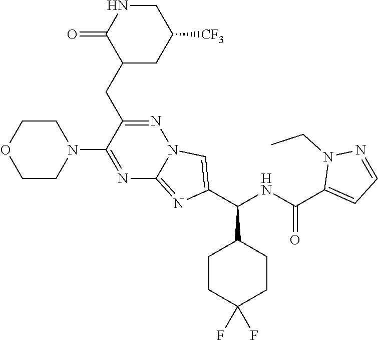 [SMILES: CCn1nccc1C(=O)N[C@H](c1cn2nc(CC3C[C@@H](C(F)(F)F)CNC3=O)c(N3CCOCC3)nc2n1)C1CCC(F)(F)CC1]
19 m/z = 654.4 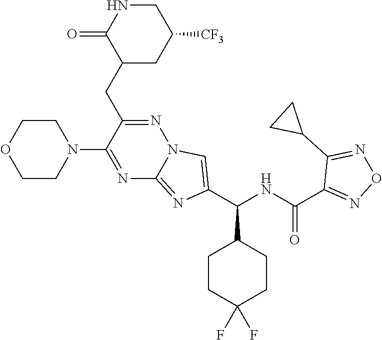 [SMILES: O=C(N[C@H](c1cn2nc(CC3C[C@@H](C(F)(F)F)CNC3=O)c(N3CCOCC3)nc2n1)C1CCC(F)(F)CC1)c1nonc1C1CC1]
20 m/z = 668.4 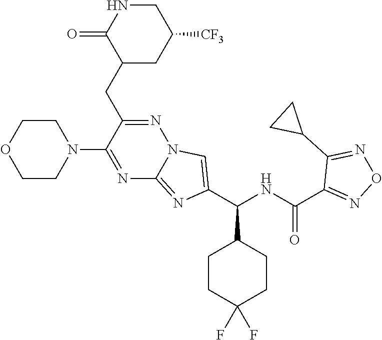 [SMILES: O=C(N[C@H](c1cn2nc(CC3C[C@@H](C(F)(F)F)CNC3=O)c(N3CCOCC3)nc2n1)C1CCC(F)(F)CC1)c1nonc1C1CC1]
21 m/z = 668.4 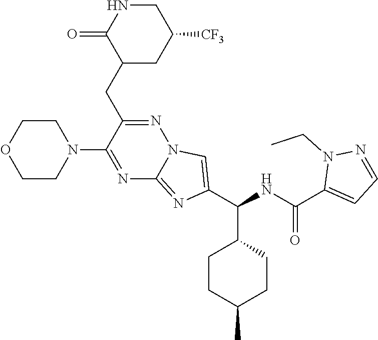 [SMILES: CCn1nccc1C(=O)N[C@H](c1cn2nc(CC3C[C@@H](C(F)(F)F)CNC3=O)c(N3CCOCC3)nc2n1)[C@H]1CC[C@H](C)CC1]
22 m/z = 632.4 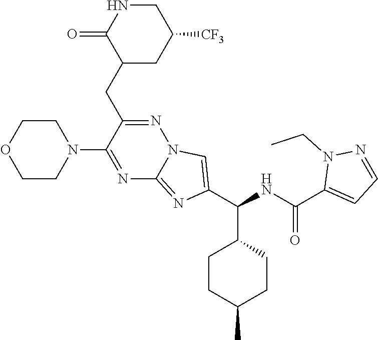 [SMILES: CCn1nccc1C(=O)N[C@H](c1cn2nc(CC3C[C@@H](C(F)(F)F)CNC3=O)c(N3CCOCC3)nc2n1)[C@H]1CC[C@H](C)CC1]
23 m/z = 632.4 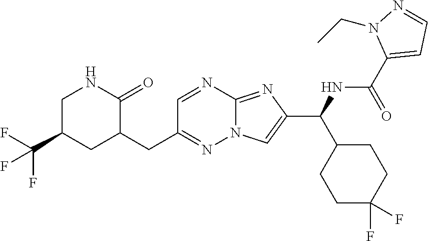 [SMILES: CCn1nccc1C(=O)N[C@H](c1cn2nc(CC3C[C@@H](C(F)(F)F)CNC3=O)cnc2n1)C1CCC(F)(F)CC1]
24 m/z = 569.5 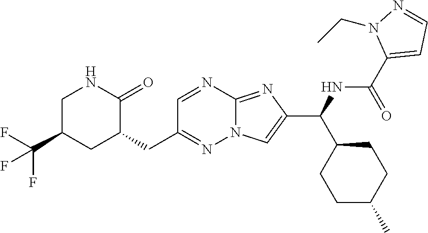 [SMILES: CCn1nccc1C(=O)N[C@H](c1cn2nc(C[C@@H]3C[C@@H](C(F)(F)F)CNC3=O)cnc2n1)[C@H]1CC[C@H](C)CC1]
25 m/z = 547.3 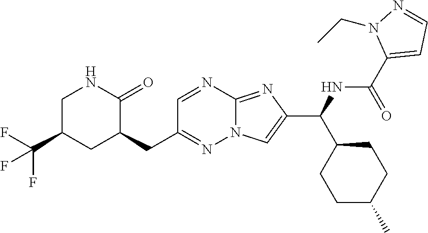 [SMILES: CCn1nccc1C(=O)N[C@H](c1cn2nc(C[C@H]3C[C@@H](C(F)(F)F)CNC3=O)cnc2n1)[C@H]1CC[C@H](C)CC1]
26 m/z = 547.3 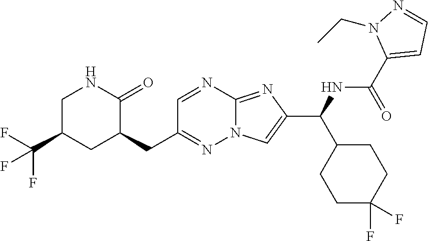 [SMILES: CCn1nccc1C(=O)N[C@H](c1cn2nc(C[C@H]3C[C@@H](C(F)(F)F)CNC3=O)cnc2n1)C1CCC(F)(F)CC1]
27 m/z = 569.3 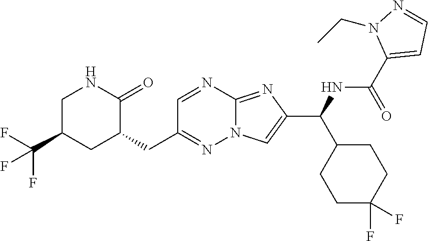 [SMILES: CCn1nccc1C(=O)N[C@H](c1cn2nc(C[C@@H]3C[C@@H](C(F)(F)F)CNC3=O)cnc2n1)C1CCC(F)(F)CC1]
28 m/z = 569.3 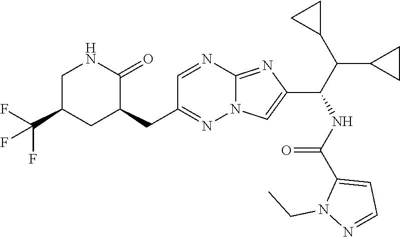 [SMILES: CCn1nccc1C(=O)N[C@H](c1cn2nc(C[C@H]3C[C@@H](C(F)(F)F)CNC3=O)cnc2n1)C(C1CC1)C1CC1]
29 m/z = 545.3 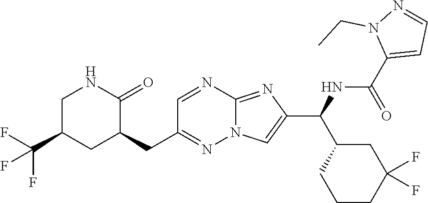 [SMILES: CCn1nccc1C(=O)N[C@H](c1cn2nc(C[C@H]3C[C@@H](C(F)(F)F)CNC3=O)cnc2n1)[C@H]1CCCC(F)(F)C1]
30 m/z = 569.3 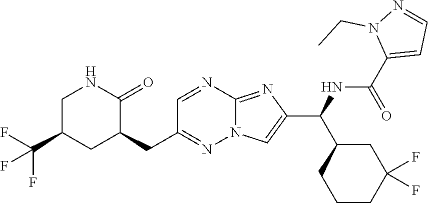 [SMILES: CCn1nccc1C(=O)N[C@H](c1cn2nc(C[C@H]3C[C@@H](C(F)(F)F)CNC3=O)cnc2n1)[C@@H]1CCCC(F)(F)C1]
31 m/z = 569.3 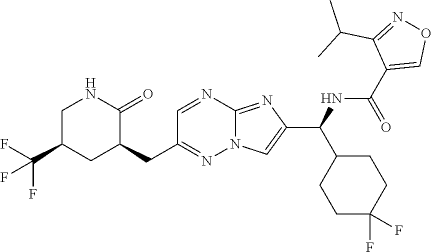 [SMILES: CC(C)c1nocc1C(=O)N[C@H](c1cn2nc(C[C@H]3C[C@@H](C(F)(F)F)CNC3=O)cnc2n1)C1CCC(F)(F)CC1]
32 m/z = 584.3 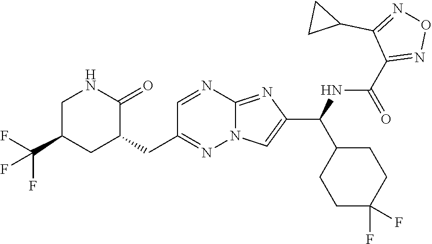 [SMILES: O=C(N[C@H](c1cn2nc(C[C@@H]3C[C@@H](C(F)(F)F)CNC3=O)cnc2n1)C1CCC(F)(F)CC1)c1nonc1C1CC1]
33 m/z = 583.2 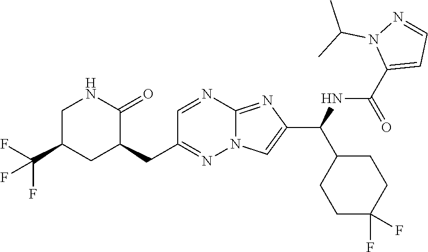 [SMILES: CC(C)n1nccc1C(=O)N[C@H](c1cn2nc(C[C@H]3C[C@@H](C(F)(F)F)CNC3=O)cnc2n1)C1CCC(F)(F)CC1]
34 m/z = 583.3 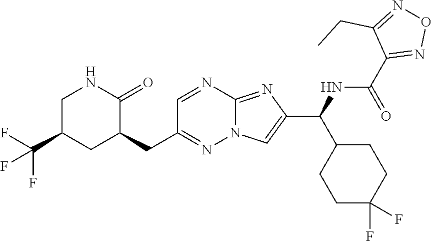 [SMILES: CCc1nonc1C(=O)N[C@H](c1cn2nc(C[C@H]3C[C@@H](C(F)(F)F)CNC3=O)cnc2n1)C1CCC(F)(F)CC1]
35 m/z = 571.3 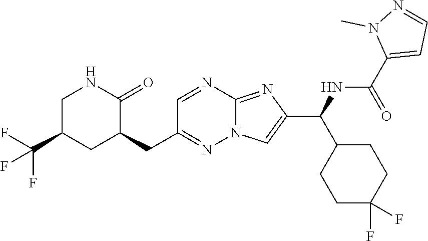 [SMILES: Cn1nccc1C(=O)N[C@H](c1cn2nc(C[C@H]3C[C@@H](C(F)(F)F)CNC3=O)cnc2n1)C1CCC(F)(F)CC1]
36 m/z = 555.2 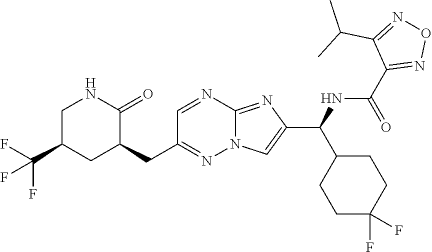 [SMILES: CC(C)c1nonc1C(=O)N[C@H](c1cn2nc(C[C@H]3C[C@@H](C(F)(F)F)CNC3=O)cnc2n1)C1CCC(F)(F)CC1]
37 m/z = 585.2 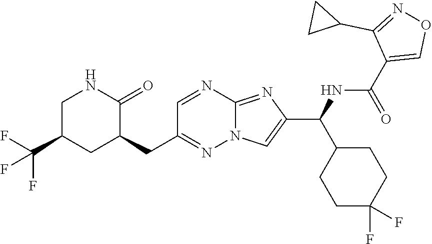 [SMILES: O=C(N[C@H](c1cn2nc(C[C@H]3C[C@@H](C(F)(F)F)CNC3=O)cnc2n1)C1CCC(F)(F)CC1)c1conc1C1CC1]
38 m/z = 582.3 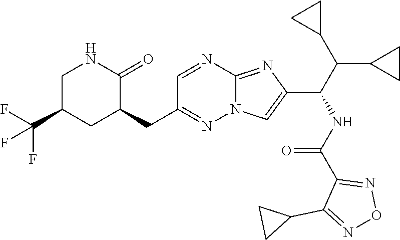 [SMILES: O=C(N[C@H](c1cn2nc(C[C@H]3C[C@@H](C(F)(F)F)CNC3=O)cnc2n1)C(C1CC1)C1CC1)c1nonc1C1CC1]
39 m/z = 559.2 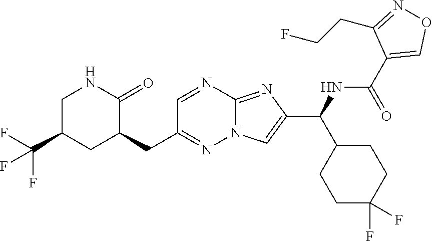 [SMILES: O=C(N[C@H](c1cn2nc(C[C@H]3C[C@@H](C(F)(F)F)CNC3=O)cnc2n1)C1CCC(F)(F)CC1)c1conc1CCF]
40 m/z = 588.3 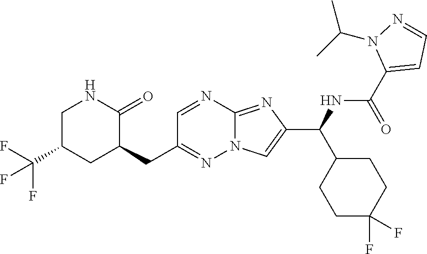 [SMILES: CC(C)n1nccc1C(=O)N[C@H](c1cn2nc(C[C@H]3C[C@H](C(F)(F)F)CNC3=O)cnc2n1)C1CCC(F)(F)CC1]
41 m/z = 583.4 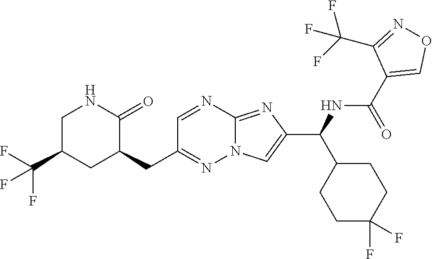 [SMILES: O=C(N[C@H](c1cn2nc(C[C@H]3C[C@@H](C(F)(F)F)CNC3=O)cnc2n1)C1CCC(F)(F)CC1)c1conc1C(F)(F)F]
42 m/z = 610.1 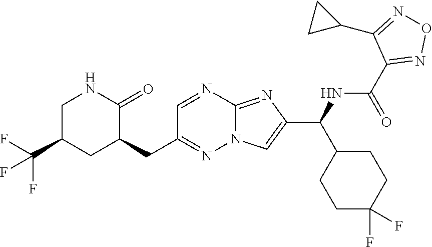 [SMILES: O=C(N[C@H](c1cn2nc(C[C@H]3C[C@@H](C(F)(F)F)CNC3=O)cnc2n1)C1CCC(F)(F)CC1)c1nonc1C1CC1]
43 m/z = 583.4 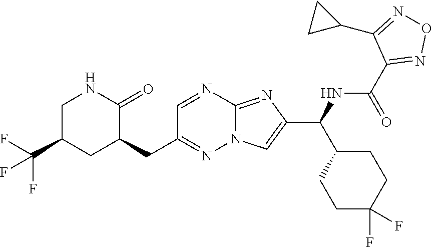
44 m/z = 583.3 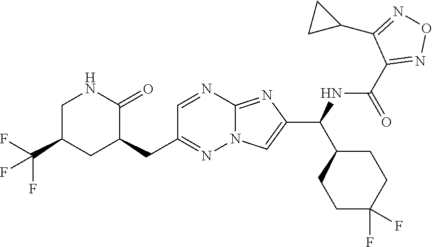
45 m/z = 583.4 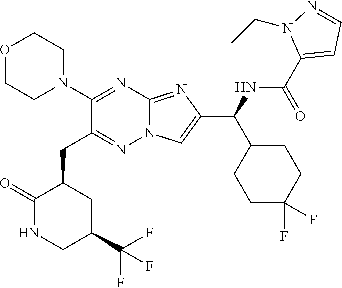 [SMILES: CCn1nccc1C(=O)N[C@H](c1cn2nc(C[C@H]3C[C@@H](C(F)(F)F)CNC3=O)c(N3CCOCC3)nc2n1)C1CCC(F)(F)CC1]
46 m/z = 654.4 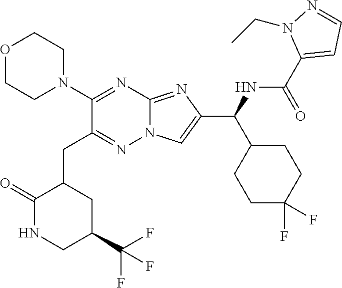 [SMILES: CCn1nccc1C(=O)N[C@H](c1cn2nc(CC3C[C@@H](C(F)(F)F)CNC3=O)c(N3CCOCC3)nc2n1)C1CCC(F)(F)CC1]
47 m/z = 654.4 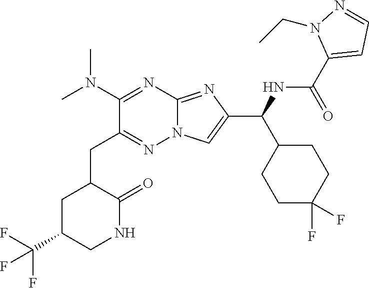 [SMILES: CCn1nccc1C(=O)N[C@H](c1cn2nc(CC3C[C@@H](C(F)(F)F)CNC3=O)c(N(C)C)nc2n1)C1CCC(F)(F)CC1]
48 m/z = 612.3 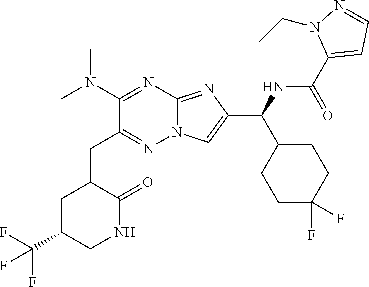 [SMILES: CCn1nccc1C(=O)N[C@H](c1cn2nc(CC3C[C@@H](C(F)(F)F)CNC3=O)c(N(C)C)nc2n1)C1CCC(F)(F)CC1]
49 m/z = 612.3 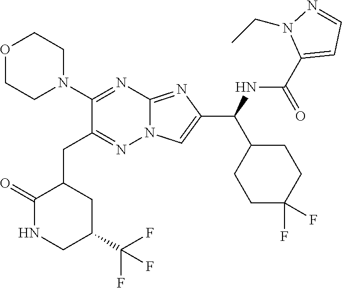 [SMILES: CCn1nccc1C(=O)N[C@H](c1cn2nc(CC3C[C@H](C(F)(F)F)CNC3=O)c(N3CCOCC3)nc2n1)C1CCC(F)(F)CC1]
50 m/z = 654.4 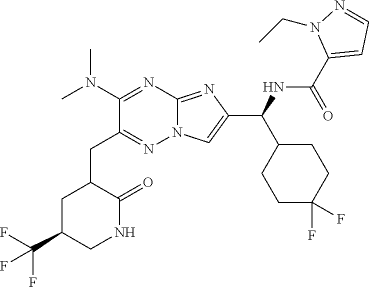 [SMILES: CCn1nccc1C(=O)N[C@H](c1cn2nc(CC3C[C@H](C(F)(F)F)CNC3=O)c(N(C)C)nc2n1)C1CCC(F)(F)CC1]
51 m/z = 612.3 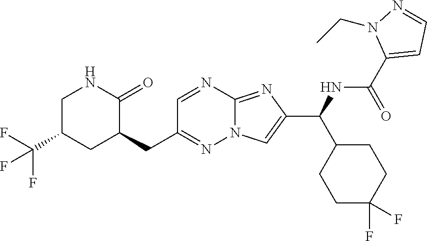 [SMILES: CCn1nccc1C(=O)N[C@H](c1cn2nc(C[C@H]3C[C@H](C(F)(F)F)CNC3=O)cnc2n1)C1CCC(F)(F)CC1]
52 m/z = 569.4 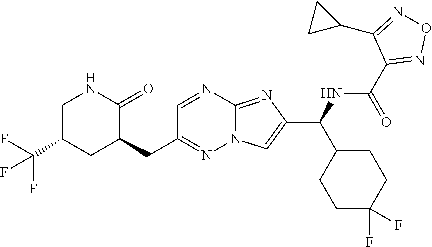 [SMILES: O=C(N[C@H](c1cn2nc(C[C@H]3C[C@H](C(F)(F)F)CNC3=O)cnc2n1)C1CCC(F)(F)CC1)c1nonc1C1CC1]
53 m/z = 583.4 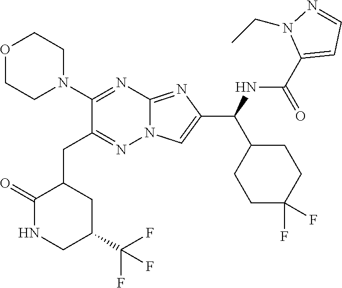 [SMILES: CCn1nccc1C(=O)N[C@H](c1cn2nc(CC3C[C@H](C(F)(F)F)CNC3=O)c(N3CCOCC3)nc2n1)C1CCC(F)(F)CC1]
54 m/z = 654.4 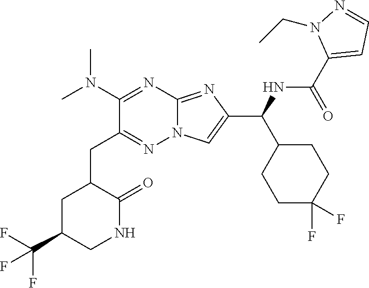 [SMILES: CCn1nccc1C(=O)N[C@H](c1cn2nc(CC3C[C@H](C(F)(F)F)CNC3=O)c(N(C)C)nc2n1)C1CCC(F)(F)CC1]
55 m/z = 612.2 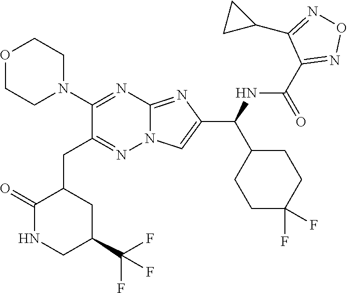 [SMILES: O=C(N[C@H](c1cn2nc(CC3C[C@@H](C(F)(F)F)CNC3=O)c(N3CCOCC3)nc2n1)C1CCC(F)(F)CC1)c1nonc1C1CC1]
56 m/z = 668.4 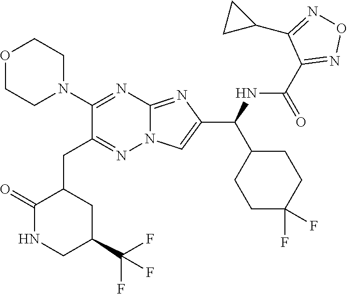 [SMILES: O=C(N[C@H](c1cn2nc(CC3C[C@@H](C(F)(F)F)CNC3=O)c(N3CCOCC3)nc2n1)C1CCC(F)(F)CC1)c1nonc1C1CC1]
57 m/z = 668.4 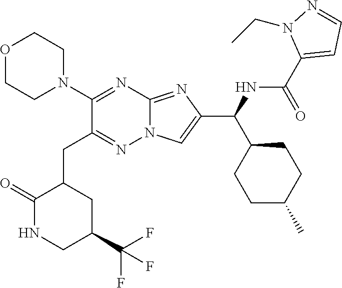 [SMILES: CCn1nccc1C(=O)N[C@H](c1cn2nc(CC3C[C@@H](C(F)(F)F)CNC3=O)c(N3CCOCC3)nc2n1)[C@H]1CC[C@H](C)CC1]
58 m/z = 632.4 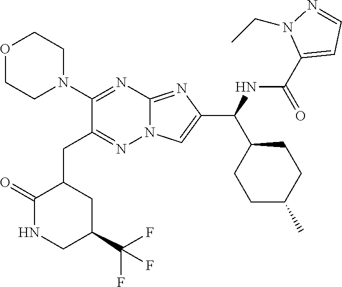 [SMILES: CCn1nccc1C(=O)N[C@H](c1cn2nc(CC3C[C@@H](C(F)(F)F)CNC3=O)c(N3CCOCC3)nc2n1)[C@H]1CC[C@H](C)CC1]
59 m/z = 632.4 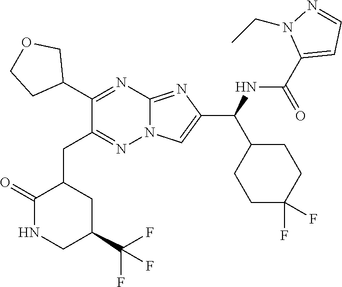 [SMILES: CCn1nccc1C(=O)N[C@H](c1cn2nc(CC3C[C@@H](C(F)(F)F)CNC3=O)c(C3CCOC3)nc2n1)C1CCC(F)(F)CC1]
60 m/z = 587.3 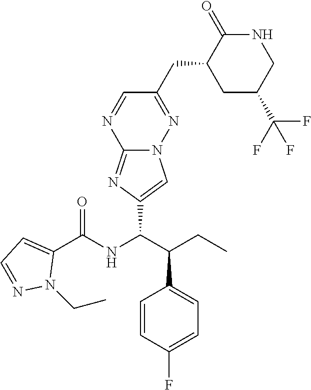 [SMILES: CC[C@@H](c1ccc(F)cc1)[C@H](NC(=O)c1ccnn1CC)c1cn2nc(C[C@H]3C[C@@H](C(F)(F)F)CNC3=O)cnc2n1]
61 m/z = 639.4 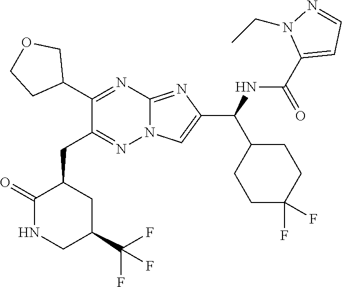 [SMILES: CCn1nccc1C(=O)N[C@H](c1cn2nc(C[C@H]3C[C@@H](C(F)(F)F)CNC3=O)c(C3CCOC3)nc2n1)C1CCC(F)(F)CC1]
62 m/z = 639.4 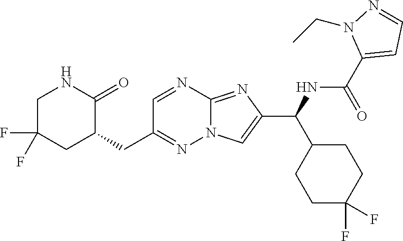 [SMILES: CCn1nccc1C(=O)N[C@H](c1cn2nc(C[C@@H]3CC(F)(F)CNC3=O)cnc2n1)C1CCC(F)(F)CC1]
63 m/z = 537.3 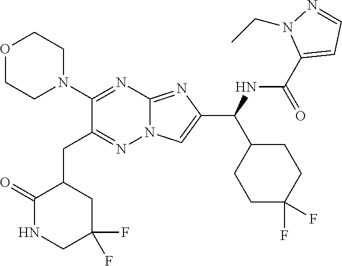 [SMILES: CCn1nccc1C(=O)N[C@H](c1cn2nc(CC3CC(F)(F)CNC3=O)c(N3CCOCC3)nc2n1)C1CCC(F)(F)CC1]
64 m/z = 622.4 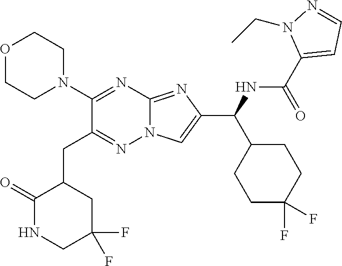 [SMILES: CCn1nccc1C(=O)N[C@H](c1cn2nc(CC3CC(F)(F)CNC3=O)c(N3CCOCC3)nc2n1)C1CCC(F)(F)CC1]
65 m/z = 622.3 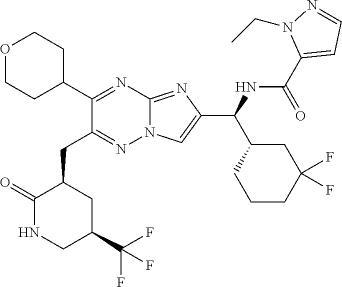 [SMILES: CCn1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]
66 m/z = 653.3 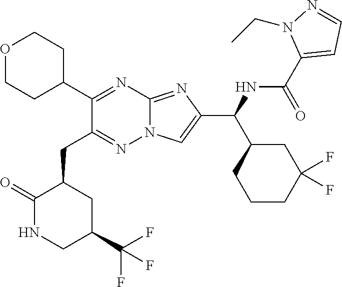 [SMILES: CCn1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]
67 m/z = 653.3 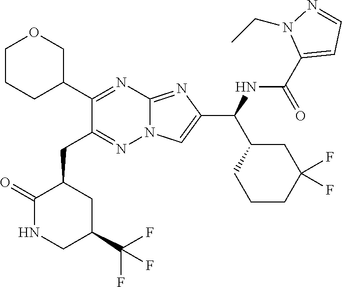 [SMILES: CCn1nccc1C(=O)N[C@H](c1cn2nc(C[C@H]3C[C@@H](C(F)(F)F)CNC3=O)c(C3CCCOC3)nc2n1)[C@H]1CCCC(F)(F)C1]
68 m/z = 653.2 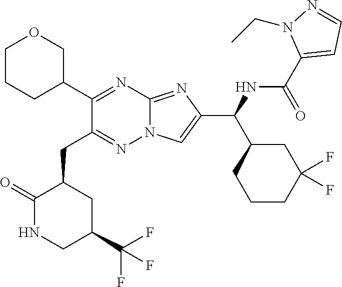 [SMILES: CCn1nccc1C(=O)N[C@H](c1cn2nc(C[C@H]3C[C@@H](C(F)(F)F)CNC3=O)c(C3CCCOC3)nc2n1)[C@@H]1CCCC(F)(F)C1]
69 m/z = 653.3 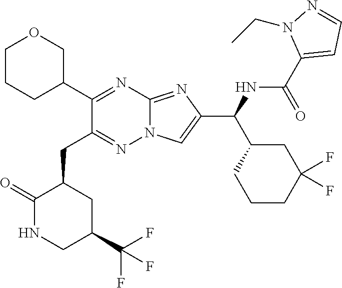 [SMILES: CCn1nccc1C(=O)N[C@H](c1cn2nc(C[C@H]3C[C@@H](C(F)(F)F)CNC3=O)c(C3CCCOC3)nc2n1)[C@H]1CCCC(F)(F)C1]
70 m/z = 653.2 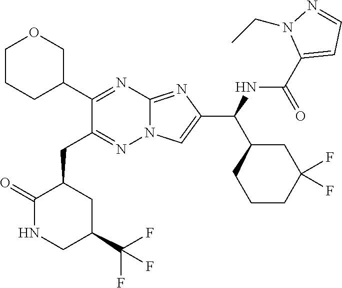 [SMILES: CCn1nccc1C(=O)N[C@H](c1cn2nc(C[C@H]3C[C@@H](C(F)(F)F)CNC3=O)c(C3CCCOC3)nc2n1)[C@@H]1CCCC(F)(F)C1]
71 m/z = 653.3 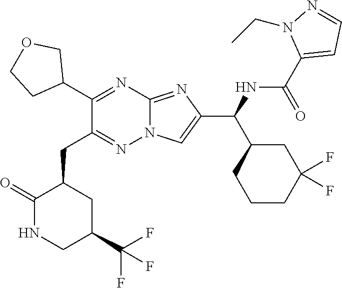 [SMILES: CCn1nccc1C(=O)N[C@H](c1cn2nc(C[C@H]3C[C@@H](C(F)(F)F)CNC3=O)c(C3CCOC3)nc2n1)[C@@H]1CCCC(F)(F)C1]
72 m/z = 639.4 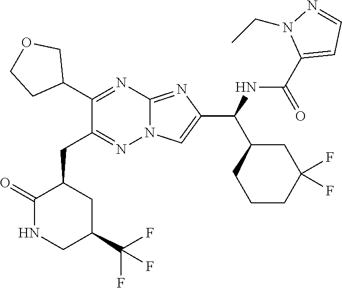 [SMILES: CCn1nccc1C(=O)N[C@H](c1cn2nc(C[C@H]3C[C@@H](C(F)(F)F)CNC3=O)c(C3CCOC3)nc2n1)[C@@H]1CCCC(F)(F)C1]
73 m/z = 639.4 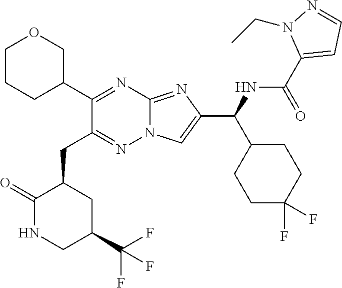 [SMILES: CCn1nccc1C(=O)N[C@H](c1cn2nc(C[C@H]3C[C@@H](C(F)(F)F)CNC3=O)c(C3CCCOC3)nc2n1)C1CCC(F)(F)CC1]
74 m/z = 653.4 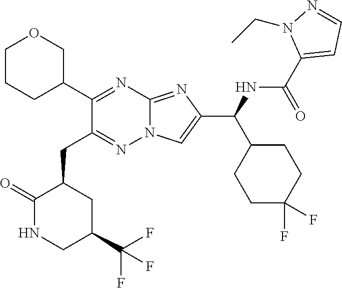 [SMILES: CCn1nccc1C(=O)N[C@H](c1cn2nc(C[C@H]3C[C@@H](C(F)(F)F)CNC3=O)c(C3CCCOC3)nc2n1)C1CCC(F)(F)CC1]
75 m/z = 653.4 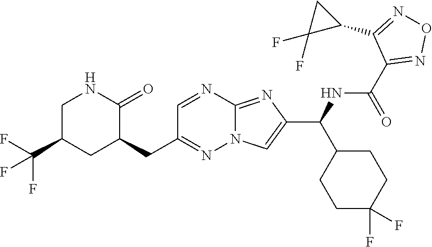 [SMILES: O=C(N[C@H](c1cn2nc(C[C@H]3C[C@@H](C(F)(F)F)CNC3=O)cnc2n1)C1CCC(F)(F)CC1)c1nonc1[C@H]1CC1(F)F]
76 m/z = 619.2 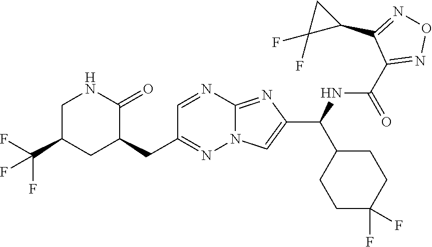 [SMILES: O=C(N[C@H](c1cn2nc(C[C@H]3C[C@@H](C(F)(F)F)CNC3=O)cnc2n1)C1CCC(F)(F)CC1)c1nonc1[C@@H]1CC1(F)F]
77 m/z = 619.2 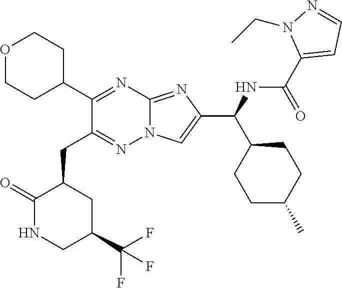 [SMILES: CCn1nccc1C(=O)N[C@H](c1cn2nc(C[C@H]3C[C@@H](C(F)(F)F)CNC3=O)c(C3CCOCC3)nc2n1)[C@H]1CC[C@H](C)CC1]
78 m/z = 631.5 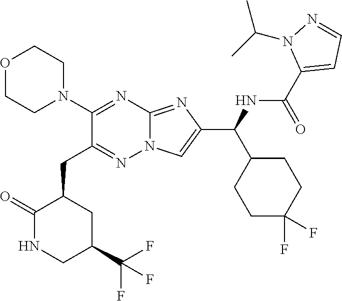 [SMILES: CC(C)n1nccc1C(=O)N[C@H](c1cn2nc(C[C@H]3C[C@@H](C(F)(F)F)CNC3=O)c(N3CCOCC3)nc2n1)C1CCC(F)(F)CC1]
79 m/z = 668.3 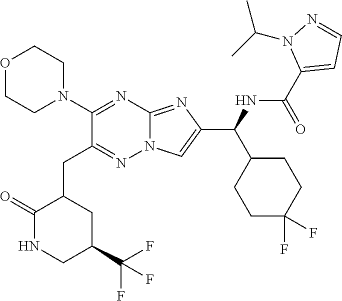 [SMILES: CC(C)n1nccc1C(=O)N[C@H](c1cn2nc(CC3C[C@@H](C(F)(F)F)CNC3=O)c(N3CCOCC3)nc2n1)C1CCC(F)(F)CC1]
80 m/z = 668.3 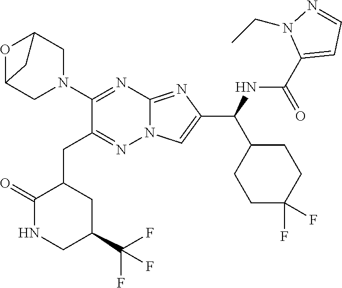 [SMILES: CCn1nccc1C(=O)N[C@H](c1cn2nc(CC3C[C@@H](C(F)(F)F)CNC3=O)c(N3CC4CC(C3)O4)nc2n1)C1CCC(F)(F)CC1]
81 m/z = 666.3 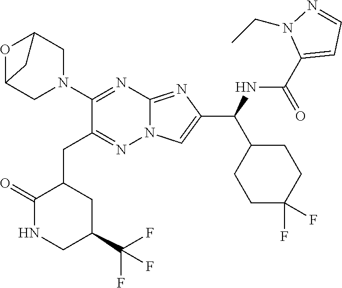 [SMILES: CCn1nccc1C(=O)N[C@H](c1cn2nc(CC3C[C@@H](C(F)(F)F)CNC3=O)c(N3CC4CC(C3)O4)nc2n1)C1CCC(F)(F)CC1]
82 m/z = 666.3 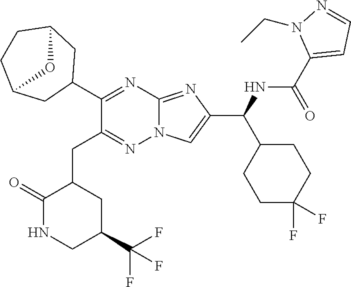 [SMILES: CCn1nccc1C(=O)N[C@H](c1cn2nc(CC3C[C@@H](C(F)(F)F)CNC3=O)c(C3C[C@H]4CC[C@@H](C3)O4)nc2n1)C1CCC(F)(F)CC1]
83 m/z = 680.3 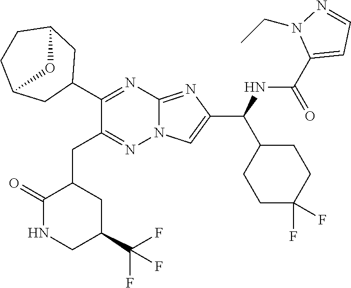 [SMILES: CCn1nccc1C(=O)N[C@H](c1cn2nc(CC3C[C@@H](C(F)(F)F)CNC3=O)c(C3C[C@H]4CC[C@@H](C3)O4)nc2n1)C1CCC(F)(F)CC1]
84 m/z = 680.3 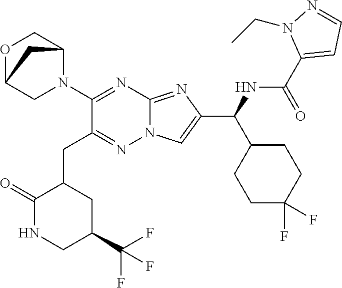 [SMILES: CCn1nccc1C(=O)N[C@H](c1cn2nc(CC3C[C@@H](C(F)(F)F)CNC3=O)c(N3C[C@H]4C[C@@H]3CO4)nc2n1)C1CCC(F)(F)CC1]
85 m/z = 666.4 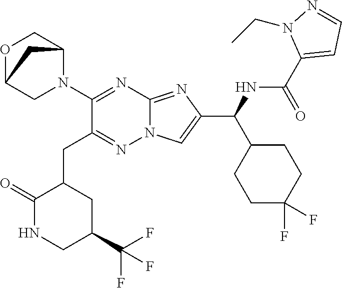 [SMILES: CCn1nccc1C(=O)N[C@H](c1cn2nc(CC3C[C@@H](C(F)(F)F)CNC3=O)c(N3C[C@H]4C[C@@H]3CO4)nc2n1)C1CCC(F)(F)CC1]
86 m/z = 666.4 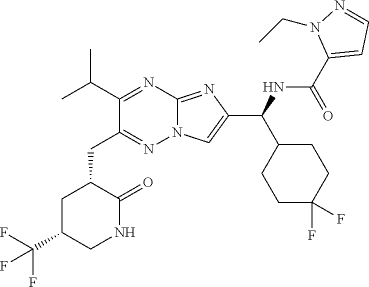 [SMILES: CCn1nccc1C(=O)N[C@H](c1cn2nc(C[C@H]3C[C@@H](C(F)(F)F)CNC3=O)c(C(C)C)nc2n1)C1CCC(F)(F)CC1]
87 m/z = 611.4 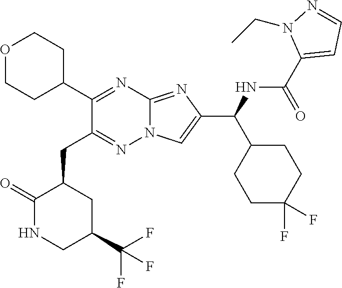 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
88 m/z = 653.4 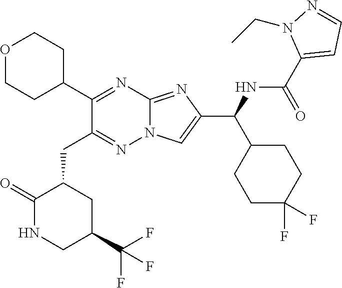 [SMILES: CCn1nccc1C(=O)N[C@H](c1cn2nc(C[C@@H]3C[C@@H](C(F)(F)F)CNC3=O)c(C3CCOCC3)nc2n1)C1CCC(F)(F)CC1]
89 m/z = 653.4 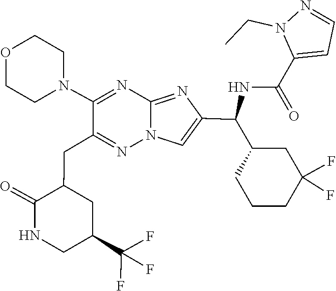 [SMILES: CCn1nccc1C(=O)N[C@H](c1cn2nc(CC3C[C@@H](C(F)(F)F)CNC3=O)c(N3CCOCC3)nc2n1)[C@H]1CCCC(F)(F)C1]
90 m/z = 654.3 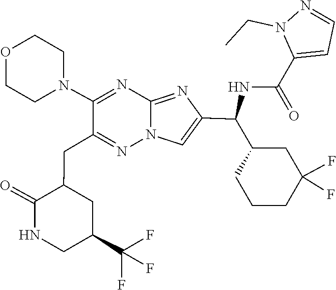 [SMILES: CCn1nccc1C(=O)N[C@H](c1cn2nc(CC3C[C@@H](C(F)(F)F)CNC3=O)c(N3CCOCC3)nc2n1)[C@H]1CCCC(F)(F)C1]
91 m/z = 654.4 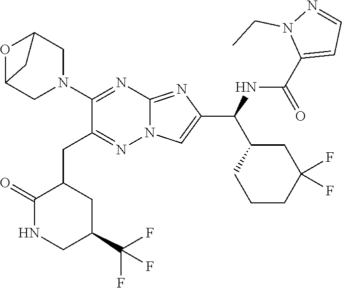 [SMILES: CCn1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]
92 m/z = 666.3 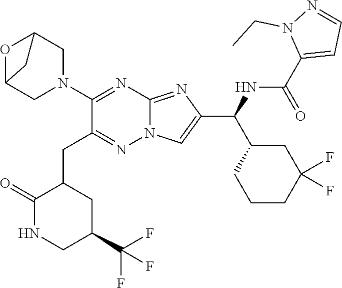 [SMILES: CCn1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]
93 m/z = 666.3 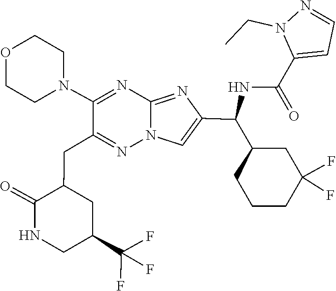 [SMILES: CCn1nccc1C(=O)N[C@H](c1cn2nc(CC3C[C@@H](C(F)(F)F)CNC3=O)c(N3CCOCC3)nc2n1)[C@@H]1CCCC(F)(F)C1]
94 m/z = 654.3 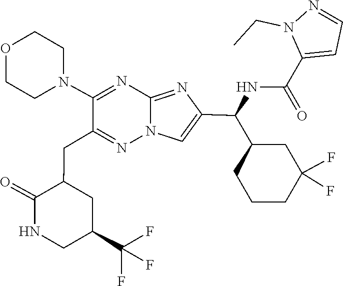 [SMILES: CCn1nccc1C(=O)N[C@H](c1cn2nc(CC3C[C@@H](C(F)(F)F)CNC3=O)c(N3CCOCC3)nc2n1)[C@@H]1CCCC(F)(F)C1]
95 m/z = 654.3 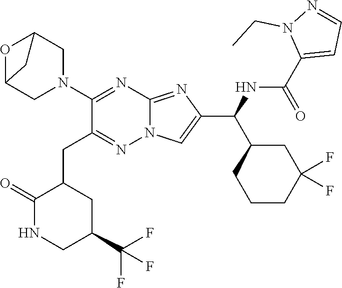 [SMILES: CCn1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]
96 m/z = 666.2 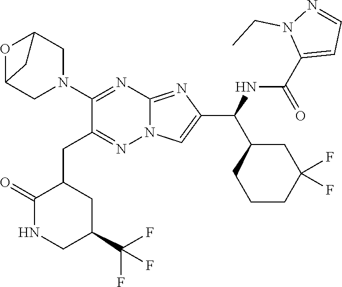 [SMILES: CCn1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]
97 m/z = 666.3 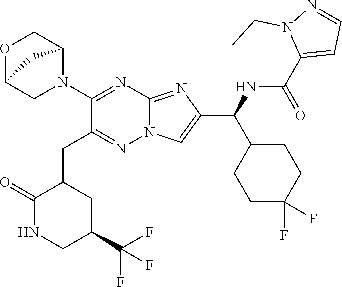 [SMILES: CCn1nccc1C(=O)N[C@H](c1cn2nc(CC3C[C@@H](C(F)(F)F)CNC3=O)c(N3C[C@@H]4C[C@H]3CO4)nc2n1)C1CCC(F)(F)CC1]
98 m/z = 666.3 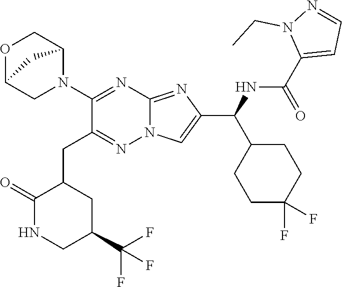 [SMILES: CCn1nccc1C(=O)N[C@H](c1cn2nc(CC3C[C@@H](C(F)(F)F)CNC3=O)c(N3C[C@@H]4C[C@H]3CO4)nc2n1)C1CCC(F)(F)CC1]
99 m/z = 666.4 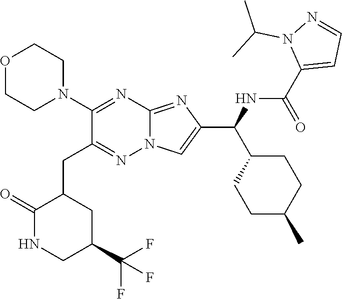 [SMILES: CC(C)n1nccc1C(=O)N[C@H](c1cn2nc(CC3C[C@@H](C(F)(F)F)CNC3=O)c(N3CCOCC3)nc2n1)[C@H]1CC[C@H](C)CC1]
100 m/z = 646.6 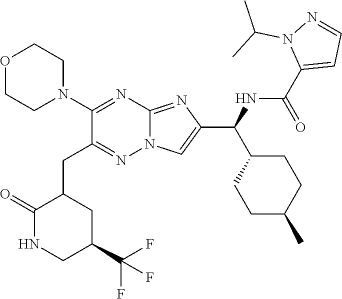 [SMILES: CC(C)n1nccc1C(=O)N[C@H](c1cn2nc(CC3C[C@@H](C(F)(F)F)CNC3=O)c(N3CCOCC3)nc2n1)[C@H]1CC[C@H](C)CC1]
101 m/z = 646.5 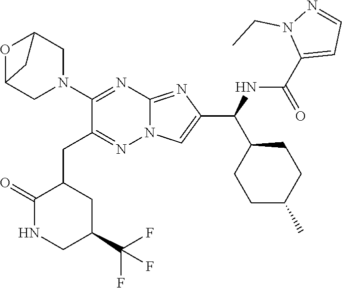 [SMILES: CCn1nccc1C(=O)N[C@H](c1cn2nc(CC3C[C@@H](C(F)(F)F)CNC3=O)c(N3CC4CC(C3)O4)nc2n1)[C@H]1CC[C@H](C)CC1]
102 m/z = 644.4 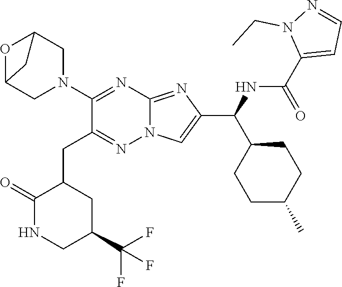 [SMILES: CCn1nccc1C(=O)N[C@H](c1cn2nc(CC3C[C@@H](C(F)(F)F)CNC3=O)c(N3CC4CC(C3)O4)nc2n1)[C@H]1CC[C@H](C)CC1]
103 m/z = 644.4 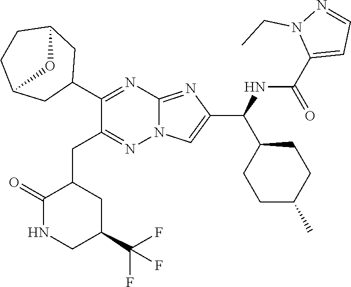 [SMILES: CCn1nccc1C(=O)N[C@H](c1cn2nc(CC3C[C@@H](C(F)(F)F)CNC3=O)c(C3C[C@H]4CC[C@@H](C3)O4)nc2n1)[C@H]1CC[C@H](C)CC1]
104 m/z = 658.3 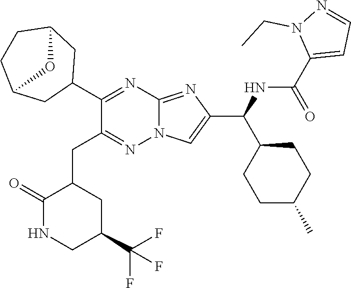 [SMILES: CCn1nccc1C(=O)N[C@H](c1cn2nc(CC3C[C@@H](C(F)(F)F)CNC3=O)c(C3C[C@H]4CC[C@@H](C3)O4)nc2n1)[C@H]1CC[C@H](C)CC1]
105 m/z = 658.3 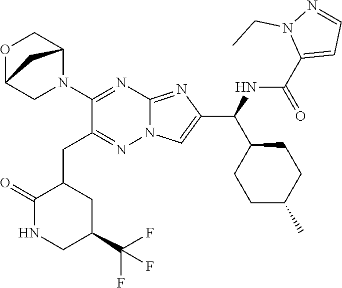 [SMILES: CCn1nccc1C(=O)N[C@H](c1cn2nc(CC3C[C@@H](C(F)(F)F)CNC3=O)c(N3C[C@H]4C[C@@H]3CO4)nc2n1)[C@H]1CC[C@H](C)CC1]
106 m/z = 644.4 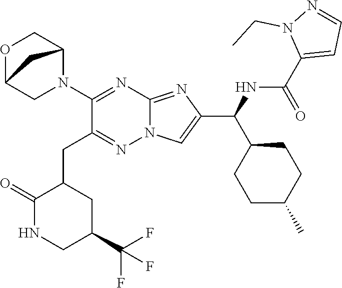 [SMILES: CCn1nccc1C(=O)N[C@H](c1cn2nc(CC3C[C@@H](C(F)(F)F)CNC3=O)c(N3C[C@H]4C[C@@H]3CO4)nc2n1)[C@H]1CC[C@H](C)CC1]
107 m/z = 644.4 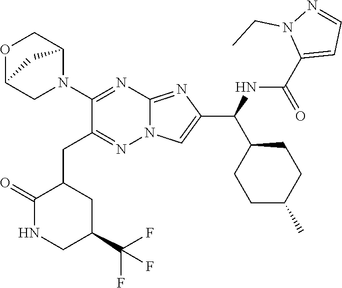 [SMILES: CCn1nccc1C(=O)N[C@H](c1cn2nc(CC3C[C@@H](C(F)(F)F)CNC3=O)c(N3C[C@@H]4C[C@H]3CO4)nc2n1)[C@H]1CC[C@H](C)CC1]
108 m/z = 644.3 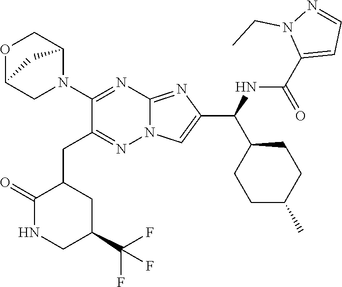 [SMILES: CCn1nccc1C(=O)N[C@H](c1cn2nc(CC3C[C@@H](C(F)(F)F)CNC3=O)c(N3C[C@@H]4C[C@H]3CO4)nc2n1)[C@H]1CC[C@H](C)CC1]
109 m/z = 644.3 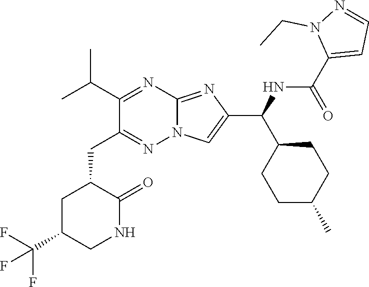 [SMILES: CCn1nccc1C(=O)N[C@H](c1cn2nc(C[C@H]3C[C@@H](C(F)(F)F)CNC3=O)c(C(C)C)nc2n1)[C@H]1CC[C@H](C)CC1]
110 m/z = 589.5 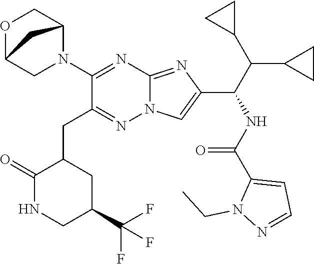 [SMILES: CCn1nccc1C(=O)N[C@H](c1cn2nc(CC3C[C@@H](C(F)(F)F)CNC3=O)c(N3C[C@H]4C[C@@H]3CO4)nc2n1)C(C1CC1)C1CC1]
111 m/z = 630.3 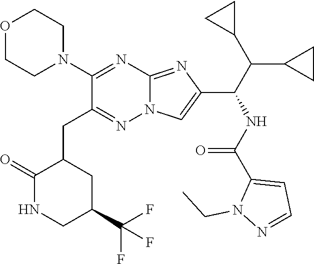 [SMILES: CCn1nccc1C(=O)N[C@H](c1cn2nc(CC3C[C@@H](C(F)(F)F)CNC3=O)c(N3CCOCC3)nc2n1)C(C1CC1)C1CC1]
112 m/z = 630.4 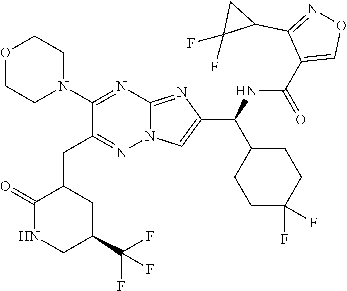 [SMILES: O=C(N[C@H](c1cn2nc(CC3C[C@@H](C(F)(F)F)CNC3=O)c(N3CCOCC3)nc2n1)C1CCC(F)(F)CC1)c1conc1C1CC1(F)F]
113 m/z = 703.2 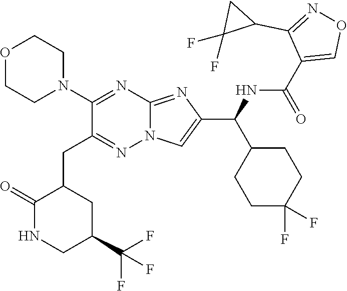 [SMILES: O=C(N[C@H](c1cn2nc(CC3C[C@@H](C(F)(F)F)CNC3=O)c(N3CCOCC3)nc2n1)C1CCC(F)(F)CC1)c1conc1C1CC1(F)F]
114 m/z = 703.3 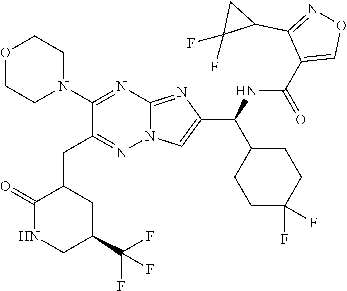 [SMILES: O=C(N[C@H](c1cn2nc(CC3C[C@@H](C(F)(F)F)CNC3=O)c(N3CCOCC3)nc2n1)C1CCC(F)(F)CC1)c1conc1C1CC1(F)F]
115 m/z = 703.2 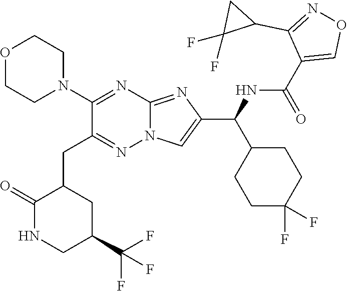 [SMILES: O=C(N[C@H](c1cn2nc(CC3C[C@@H](C(F)(F)F)CNC3=O)c(N3CCOCC3)nc2n1)C1CCC(F)(F)CC1)c1conc1C1CC1(F)F]
116 m/z = 703.2 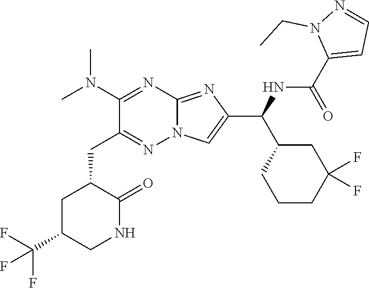 [SMILES: CCn1nccc1C(=O)N[C@H](c1cn2nc(C[C@H]3C[C@@H](C(F)(F)F)CNC3=O)c(N(C)C)nc2n1)[C@H]1CCCC(F)(F)C1]
117 m/z = 612.3 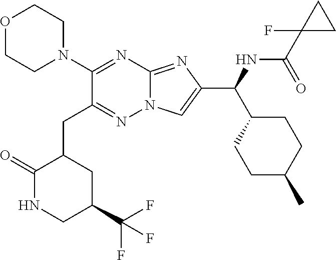 [SMILES: C[C@H]1CC[C@H]([C@H](NC(=O)C2(F)CC2)c2cn3nc(CC4C[C@@H](C(F)(F)F)CNC4=O)c(N4CCOCC4)nc3n2)CC1]
118 m/z = 596.4 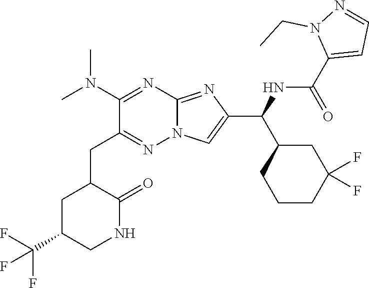 [SMILES: CCn1nccc1C(=O)N[C@H](c1cn2nc(CC3C[C@@H](C(F)(F)F)CNC3=O)c(N(C)C)nc2n1)[C@@H]1CCCC(F)(F)C1]
119 m/z = 612.4 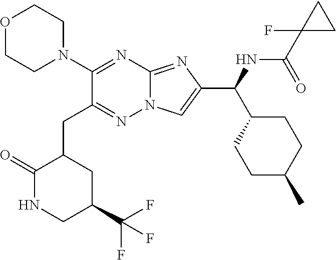 [SMILES: C[C@H]1CC[C@H]([C@H](NC(=O)C2(F)CC2)c2cn3nc(CC4C[C@@H](C(F)(F)F)CNC4=O)c(N4CCOCC4)nc3n2)CC1]
120 m/z = 596.4 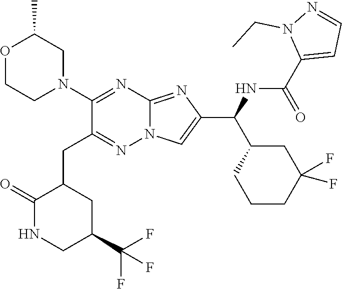 [SMILES: CCn1nccc1C(=O)N[C@H](c1cn2nc(CC3C[C@@H](C(F)(F)F)CNC3=O)c(N3CCO[C@H](C)C3)nc2n1)[C@H]1CCCC(F)(F)C1]
121 m/z = 668.5 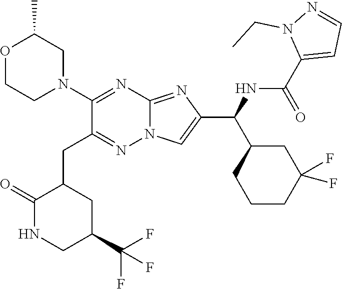 [SMILES: CCn1nccc1C(=O)N[C@H](c1cn2nc(CC3C[C@@H](C(F)(F)F)CNC3=O)c(N3CCO[C@H](C)C3)nc2n1)[C@@H]1CCCC(F)(F)C1]
122 m/z = 668.4 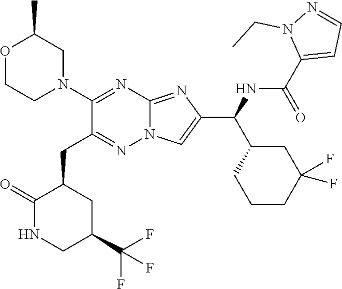 [SMILES: CCn1nccc1C(=O)N[C@H](c1cn2nc(C[C@H]3C[C@@H](C(F)(F)F)CNC3=O)c(N3CCO[C@@H](C)C3)nc2n1)[C@H]1CCCC(F)(F)C1]
123 m/z = 668.5 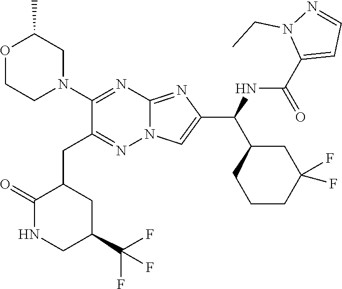 [SMILES: CCn1nccc1C(=O)N[C@H](c1cn2nc(CC3C[C@@H](C(F)(F)F)CNC3=O)c(N3CCO[C@H](C)C3)nc2n1)[C@@H]1CCCC(F)(F)C1]
124 m/z = 668.4 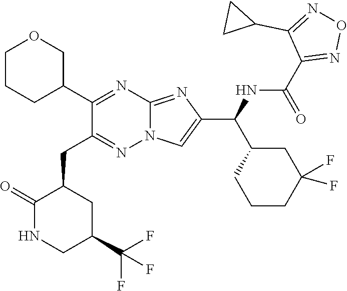 [SMILES: O=C(N[C@H](c1cn2nc(C[C@H]3C[C@@H](C(F)(F)F)CNC3=O)c(C3CCCOC3)nc2n1)[C@H]1CCCC(F)(F)C1)c1nonc1C1CC1]
125 m/z = 667.5 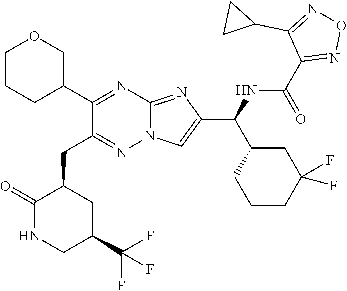 [SMILES: O=C(N[C@H](c1cn2nc(C[C@H]3C[C@@H](C(F)(F)F)CNC3=O)c(C3CCCOC3)nc2n1)[C@H]1CCCC(F)(F)C1)c1nonc1C1CC1]
126 m/z = 667.5 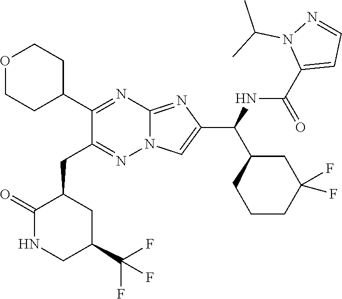 [SMILES: CC(C)n1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]
127 m/z = 667.4 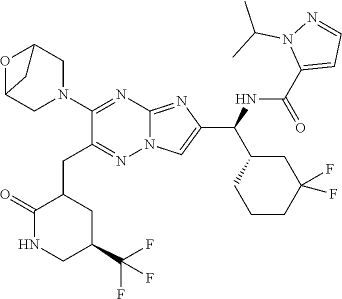 [SMILES: CC(C)n1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]
128 m/z = 680.4 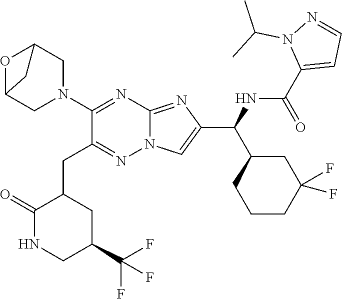 [SMILES: CC(C)n1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]
129 m/z = 680.3 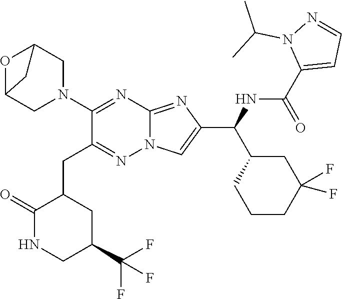 [SMILES: CC(C)n1nccc1C(=O)N[C@H](c1cn2nc(CC3C[C@@H](C(F)(F)F)CNC3=O)c(N3CC4CC(C3)O4)nc2n1)[C@H]1CCCC(F)(F)C1]
130 m/z = 680.4 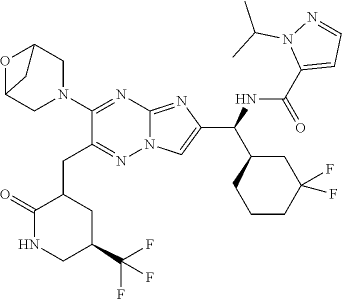 [SMILES: CC(C)n1nccc1C(=O)N[C@H](c1cn2nc(CC3C[C@@H](C(F)(F)F)CNC3=O)c(N3CC4CC(C3)O4)nc2n1)[C@@H]1CCCC(F)(F)C1]
131 m/z = 680.3 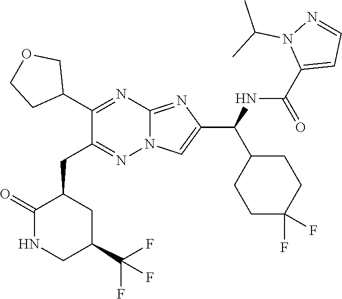 [SMILES: CC(C)n1nccc1C(=O)N[C@H](c1cn2nc(C[C@H]3C[C@@H](C(F)(F)F)CNC3=O)c(C3CCOC3)nc2n1)C1CCC(F)(F)CC1]
132 m/z = 653.4 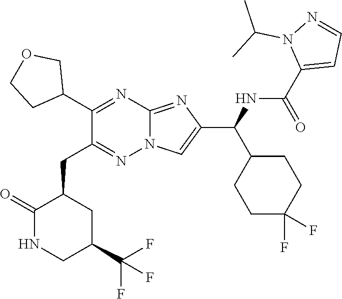 [SMILES: CC(C)n1nccc1C(=O)N[C@H](c1cn2nc(C[C@H]3C[C@@H](C(F)(F)F)CNC3=O)c(C3CCOC3)nc2n1)C1CCC(F)(F)CC1]
133 m/z = 653.4 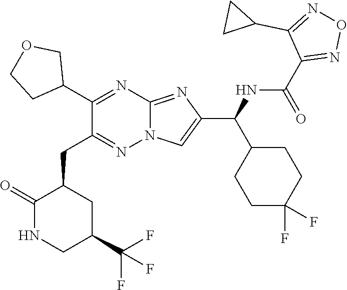 [SMILES: O=C(N[C@H](c1cn2nc(C[C@H]3C[C@@H](C(F)(F)F)CNC3=O)c(C3CCOC3)nc2n1)C1CCC(F)(F)CC1)c1nonc1C1CC1]
134 m/z = 653.4 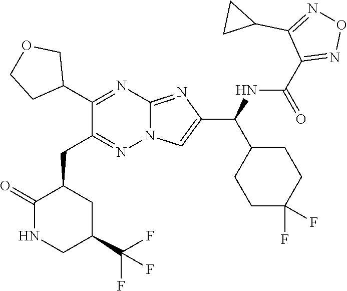 [SMILES: O=C(N[C@H](c1cn2nc(C[C@H]3C[C@@H](C(F)(F)F)CNC3=O)c(C3CCOC3)nc2n1)C1CCC(F)(F)CC1)c1nonc1C1CC1]
135 m/z = 653.4 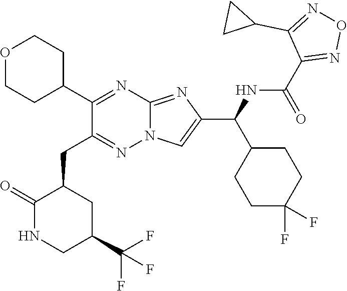 [SMILES: O=C(N[C@H](c1cn2nc(C[C@H]3C[C@@H](C(F)(F)F)CNC3=O)c(C3CCOCC3)nc2n1)C1CCC(F)(F)CC1)c1nonc1C1CC1]
136 m/z = 667.4 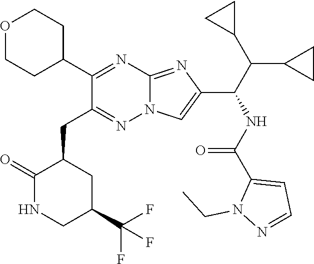 [SMILES: CCn1nccc1C(=O)N[C@H](c1cn2nc(C[C@H]3C[C@@H](C(F)(F)F)CNC3=O)c(C3CCOCC3)nc2n1)C(C1CC1)C1CC1]
137 m/z = 629.3 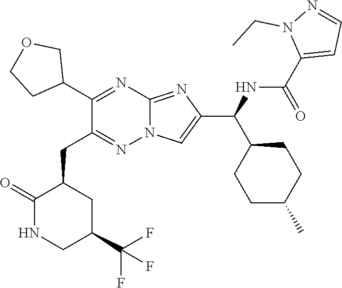 [SMILES: CCn1nccc1C(=O)N[C@H](c1cn2nc(C[C@H]3C[C@@H](C(F)(F)F)CNC3=O)c(C3CCOC3)nc2n1)[C@H]1CC[C@H](C)CC1]
138 m/z = 617.3 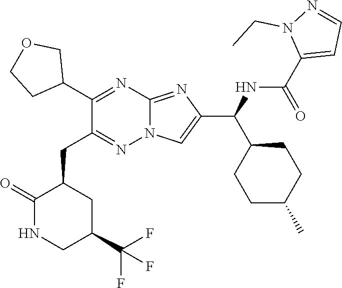 [SMILES: CCn1nccc1C(=O)N[C@H](c1cn2nc(C[C@H]3C[C@@H](C(F)(F)F)CNC3=O)c(C3CCOC3)nc2n1)[C@H]1CC[C@H](C)CC1]
139 m/z = 617.3 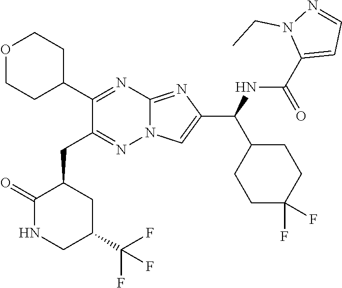 [SMILES: CCn1nccc1C(=O)N[C@H](c1cn2nc(C[C@H]3C[C@H](C(F)(F)F)CNC3=O)c(C3CCOCC3)nc2n1)C1CCC(F)(F)CC1]
140 m/z = 653.4 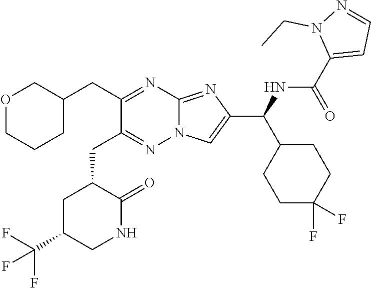 [SMILES: CCn1nccc1C(=O)N[C@H](c1cn2nc(C[C@H]3C[C@@H](C(F)(F)F)CNC3=O)c(CC3CCCOC3)nc2n1)C1CCC(F)(F)CC1]
141 m/z = 667.4 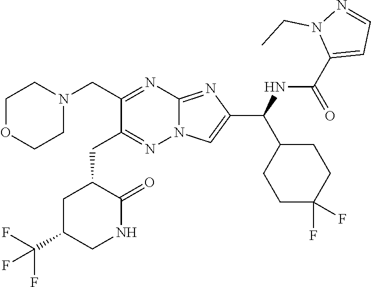 [SMILES: CCn1nccc1C(=O)N[C@H](c1cn2nc(C[C@H]3C[C@@H](C(F)(F)F)CNC3=O)c(CN3CCOCC3)nc2n1)C1CCC(F)(F)CC1]
142 m/z = 668.4 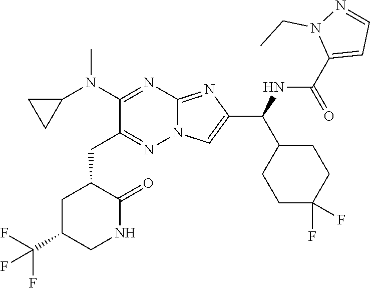 [SMILES: CCn1nccc1C(=O)N[C@H](c1cn2nc(C[C@H]3C[C@@H](C(F)(F)F)CNC3=O)c(N(C)C3CC3)nc2n1)C1CCC(F)(F)CC1]
143 m/z = 638.3 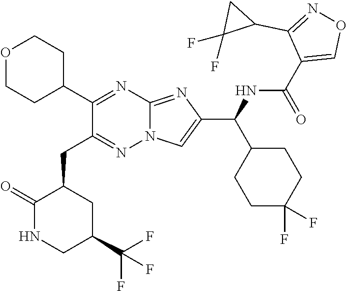 [SMILES: O=C(N[C@H](c1cn2nc(C[C@H]3C[C@@H](C(F)(F)F)CNC3=O)c(C3CCOCC3)nc2n1)C1CCC(F)(F)CC1)c1conc1C1CC1(F)F]
144 m/z = 702.3 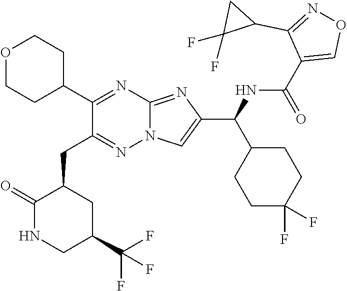 [SMILES: O=C(N[C@H](c1cn2nc(C[C@H]3C[C@@H](C(F)(F)F)CNC3=O)c(C3CCOCC3)nc2n1)C1CCC(F)(F)CC1)c1conc1C1CC1(F)F]
145 m/z = 702.3 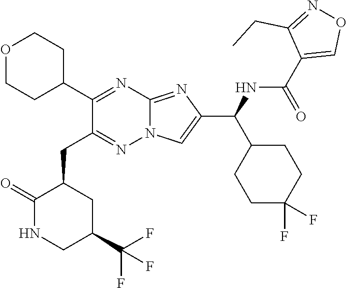 [SMILES: CCc1nocc1C(=O)N[C@H](c1cn2nc(C[C@H]3C[C@@H](C(F)(F)F)CNC3=O)c(C3CCOCC3)nc2n1)C1CCC(F)(F)CC1]
146 m/z = 654.5 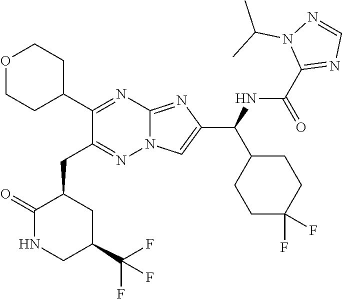 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]
147 m/z = 667.3 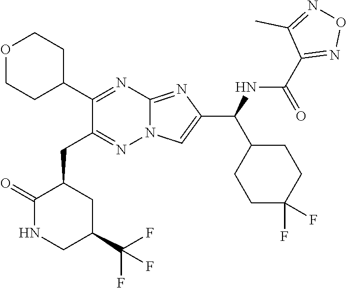 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]
148 m/z = 641.4 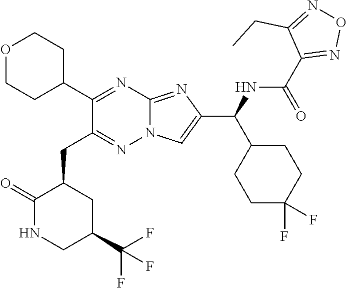 [SMILES: CCc1nonc1C(=O)N[C@H](c1cn2nc(C[C@H]3C[C@@H](C(F)(F)F)CNC3=O)c(C3CCOCC3)nc2n1)C1CCC(F)(F)CC1]
149 m/z = 655.5 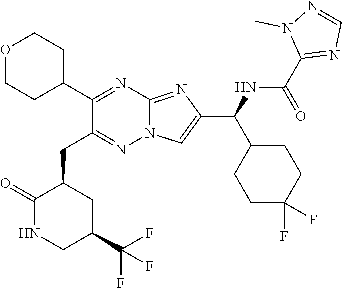 [SMILES: Cn1ncnc1C(=O)N[C@H](c1cn2nc(C[C@H]3C[C@@H](C(F)(F)F)CNC3=O)c(C3CCOCC3)nc2n1)C1CCC(F)(F)CC1]
150 m/z = 639.5 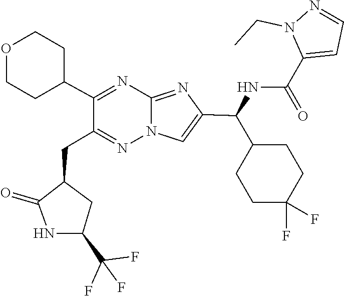 [SMILES: CCn1nccc1C(=O)N[C@H](c1cn2nc(C[C@H]3C[C@@H](C(F)(F)F)NC3=O)c(C3CCOCC3)nc2n1)C1CCC(F)(F)CC1]
151 m/z = 639.4 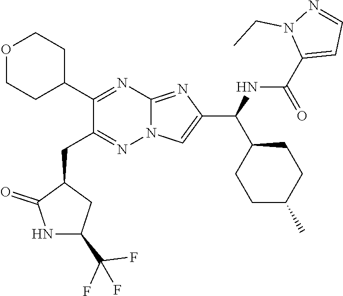 [SMILES: CCn1nccc1C(=O)N[C@H](c1cn2nc(C[C@H]3C[C@@H](C(F)(F)F)NC3=O)c(C3CCOCC3)nc2n1)[C@H]1CC[C@H](C)CC1]
152 m/z = 617.4 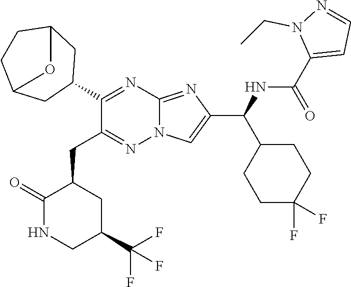
153 m/z = 679.4 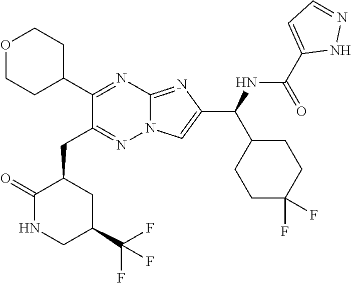 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccn[nH]1]
154 m/z = 625.3 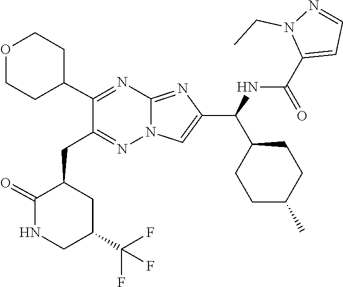 [SMILES: CCn1nccc1C(=O)N[C@H](c1cn2nc(C[C@H]3C[C@H](C(F)(F)F)CNC3=O)c(C3CCOCC3)nc2n1)[C@H]1CC[C@H](C)CC1]
155 m/z = 631.3 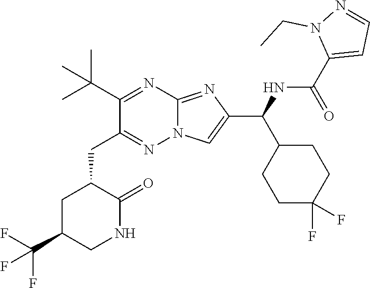 [SMILES: CCn1nccc1C(=O)N[C@H](c1cn2nc(C[C@H]3C[C@H](C(F)(F)F)CNC3=O)c(C(C)(C)C)nc2n1)C1CCC(F)(F)CC1]
156 m/z = 625.3 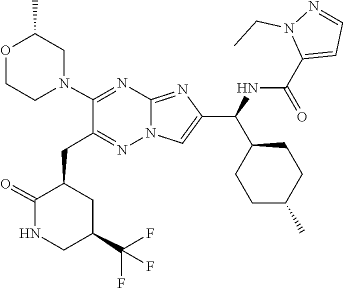 [SMILES: CCn1nccc1C(=O)N[C@H](c1cn2nc(C[C@H]3C[C@@H](C(F)(F)F)CNC3=O)c(N3CCO[C@H](C)C3)nc2n1)[C@H]1CC[C@H](C)CC1]
157 m/z = 646.4 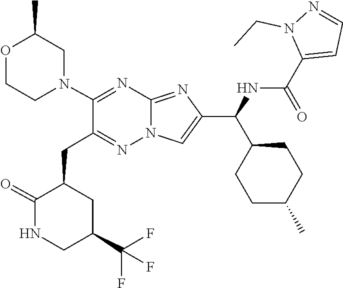 [SMILES: CCn1nccc1C(=O)N[C@H](c1cn2nc(C[C@H]3C[C@@H](C(F)(F)F)CNC3=O)c(N3CCO[C@@H](C)C3)nc2n1)[C@H]1CC[C@H](C)CC1]
158 m/z = 646.4 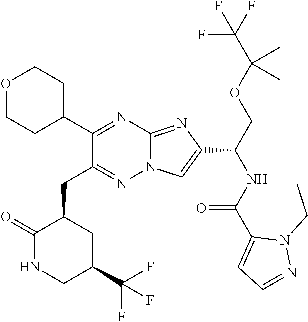 [SMILES: CCn1nccc1C(=O)N[C@@H](COC(C)(C)C(F)(F)F)c1cn2nc(C[C@H]3C[C@@H](C(F)(F)F)CNC3=O)c(C3CCOCC3)nc2n1]
159 m/z = 675.4 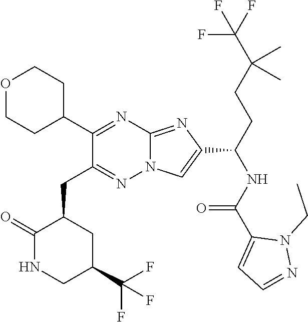 [SMILES: CCn1nccc1C(=O)N[C@@H](CCC(C)(C)C(F)(F)F)c1cn2nc(C[C@H]3C[C@@H](C(F)(F)F)CNC3=O)c(C3CCOCC3)nc2n1]
160 m/z = 673.3 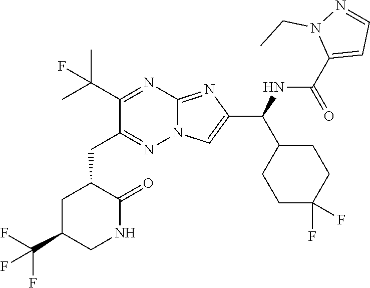 [SMILES: CCn1nccc1C(=O)N[C@H](c1cn2nc(C[C@H]3C[C@H](C(F)(F)F)CNC3=O)c(C(C)(C)F)nc2n1)C1CCC(F)(F)CC1]
161 m/z = 629.3 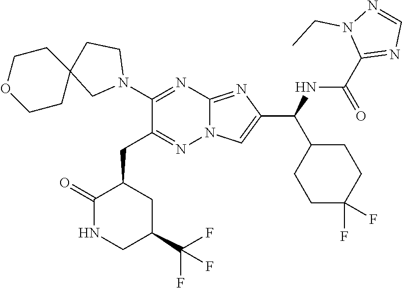 [SMILES: CCn1ncnc1C(=O)N[C@H](c1cn2nc(C[C@H]3C[C@@H](C(F)(F)F)CNC3=O)c(N3CCC4(CCOCC4)C3)nc2n1)C1CCC(F)(F)CC1]
162 m/z = 708.3 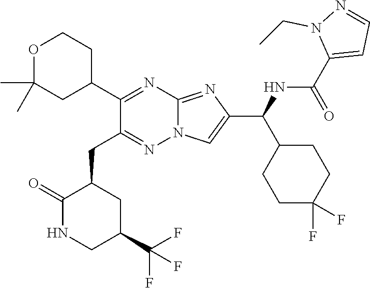 [SMILES: CCn1nccc1C(=O)N[C@H](c1cn2nc(C[C@H]3C[C@@H](C(F)(F)F)CNC3=O)c(C3CCOC(C)(C)C3)nc2n1)C1CCC(F)(F)CC1]
163 m/z = 681.3 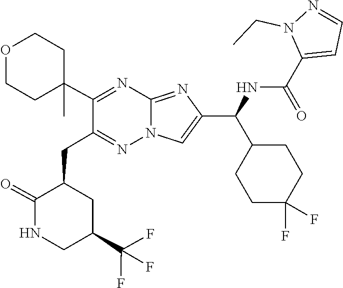 [SMILES: CCn1nccc1C(=O)N[C@H](c1cn2nc(C[C@H]3C[C@@H](C(F)(F)F)CNC3=O)c(C3(C)CCOCC3)nc2n1)C1CCC(F)(F)CC1]
164 m/z = 667.2 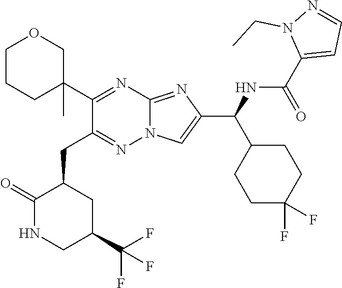 [SMILES: CCn1nccc1C(=O)N[C@H](c1cn2nc(C[C@H]3C[C@@H](C(F)(F)F)CNC3=O)c(C3(C)CCCOC3)nc2n1)C1CCC(F)(F)CC1]
165 m/z = 667.4 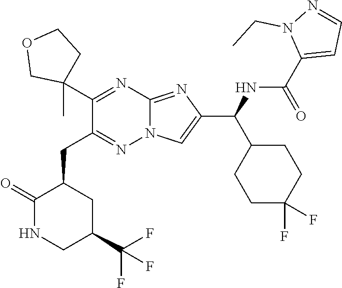 [SMILES: CCn1nccc1C(=O)N[C@H](c1cn2nc(C[C@H]3C[C@@H](C(F)(F)F)CNC3=O)c(C3(C)CCOC3)nc2n1)C1CCC(F)(F)CC1]
166 m/z = 653.3 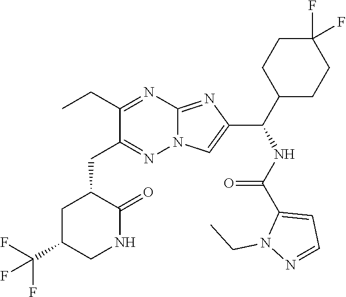 [SMILES: CCc1nc2nc([C@@H](NC(=O)c3ccnn3CC)C3CCC(F)(F)CC3)cn2nc1C[C@H]1C[C@@H](C(F)(F)F)CNC1=O]
167 m/z = 597.4 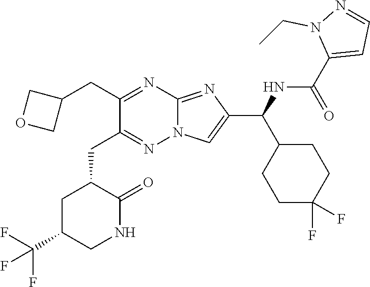 [SMILES: CCn1nccc1C(=O)N[C@H](c1cn2nc(C[C@H]3C[C@@H](C(F)(F)F)CNC3=O)c(CC3COC3)nc2n1)C1CCC(F)(F)CC1]
168 m/z = 639.3 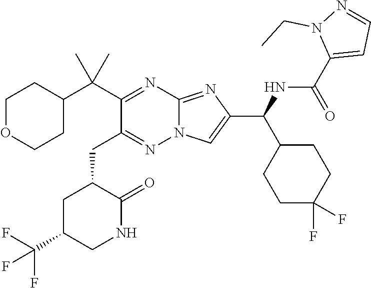 [SMILES: CCn1nccc1C(=O)N[C@H](c1cn2nc(C[C@H]3C[C@@H](C(F)(F)F)CNC3=O)c(C(C)(C)C3CCOCC3)nc2n1)C1CCC(F)(F)CC1]
169 m/z = 695.4 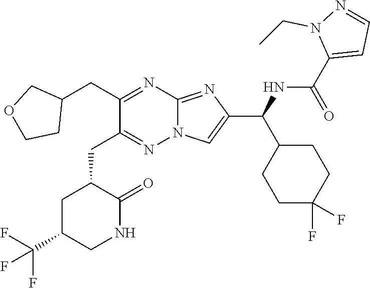 [SMILES: CCn1nccc1C(=O)N[C@H](c1cn2nc(C[C@H]3C[C@@H](C(F)(F)F)CNC3=O)c(CC3CCOC3)nc2n1)C1CCC(F)(F)CC1]
170 m/z = 653.4 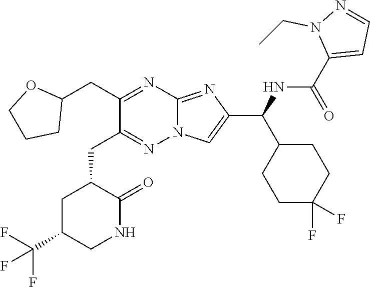 [SMILES: CCn1nccc1C(=O)N[C@H](c1cn2nc(C[C@H]3C[C@@H](C(F)(F)F)CNC3=O)c(CC3CCCO3)nc2n1)C1CCC(F)(F)CC1]
171 m/z = 653.3 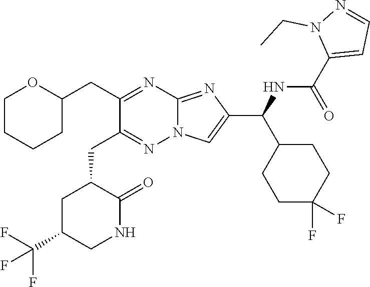 [SMILES: CCn1nccc1C(=O)N[C@H](c1cn2nc(C[C@H]3C[C@@H](C(F)(F)F)CNC3=O)c(CC3CCCCO3)nc2n1)C1CCC(F)(F)CC1]
172 m/z = 667.4 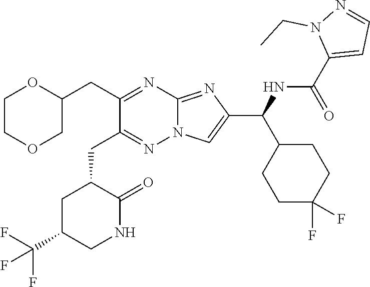 [SMILES: CCn1nccc1C(=O)N[C@H](c1cn2nc(C[C@H]3C[C@@H](C(F)(F)F)CNC3=O)c(CC3COCCO3)nc2n1)C1CCC(F)(F)CC1]
173 m/z = 669.4 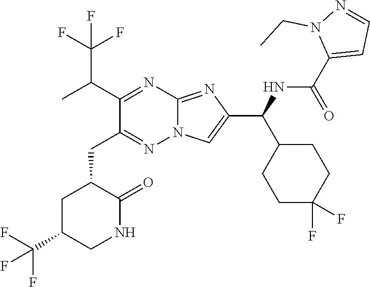 [SMILES: CCn1nccc1C(=O)N[C@H](c1cn2nc(C[C@H]3C[C@@H](C(F)(F)F)CNC3=O)c(C(C)C(F)(F)F)nc2n1)C1CCC(F)(F)CC1]
174 m/z = 665.5 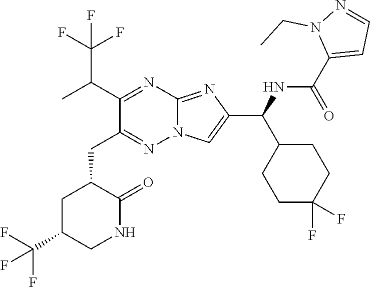 [SMILES: CCn1nccc1C(=O)N[C@H](c1cn2nc(C[C@H]3C[C@@H](C(F)(F)F)CNC3=O)c(C(C)C(F)(F)F)nc2n1)C1CCC(F)(F)CC1]
175 m/z = 665.4 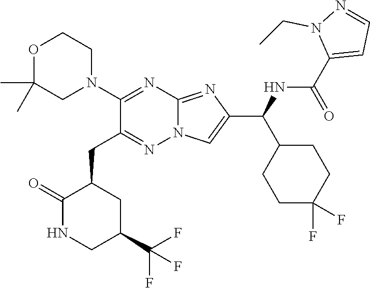 [SMILES: CCn1nccc1C(=O)N[C@H](c1cn2nc(C[C@H]3C[C@@H](C(F)(F)F)CNC3=O)c(N3CCOC(C)(C)C3)nc2n1)C1CCC(F)(F)CC1]
176 m/z = 681.3 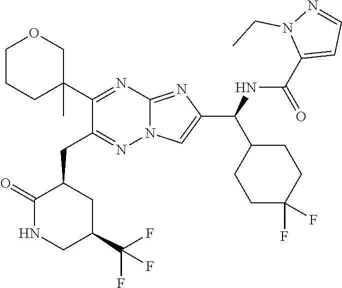 [SMILES: CCn1nccc1C(=O)N[C@H](c1cn2nc(C[C@H]3C[C@@H](C(F)(F)F)CNC3=O)c(C3(C)CCCOC3)nc2n1)C1CCC(F)(F)CC1]
177 m/z = 667.4 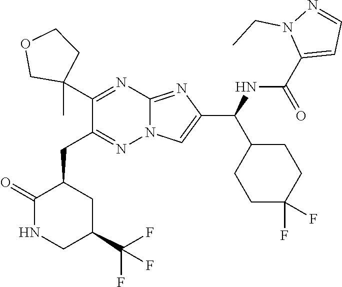 [SMILES: CCn1nccc1C(=O)N[C@H](c1cn2nc(C[C@H]3C[C@@H](C(F)(F)F)CNC3=O)c(C3(C)CCOC3)nc2n1)C1CCC(F)(F)CC1]
178 m/z = 653.3 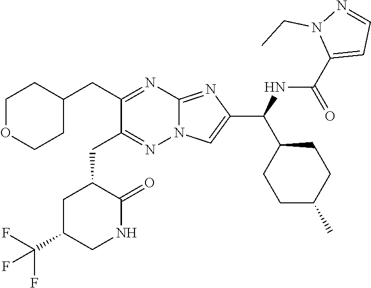 [SMILES: CCn1nccc1C(=O)N[C@H](c1cn2nc(C[C@H]3C[C@@H](C(F)(F)F)CNC3=O)c(CC3CCOCC3)nc2n1)[C@H]1CC[C@H](C)CC1]
179 m/z = 645.3 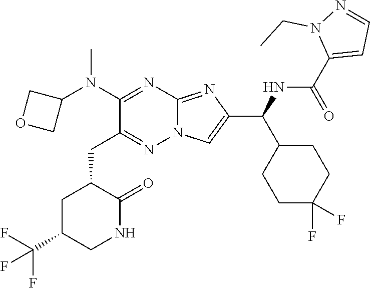 [SMILES: CCn1nccc1C(=O)N[C@H](c1cn2nc(C[C@H]3C[C@@H](C(F)(F)F)CNC3=O)c(N(C)C3COC3)nc2n1)C1CCC(F)(F)CC1]
180 m/z = 654.2 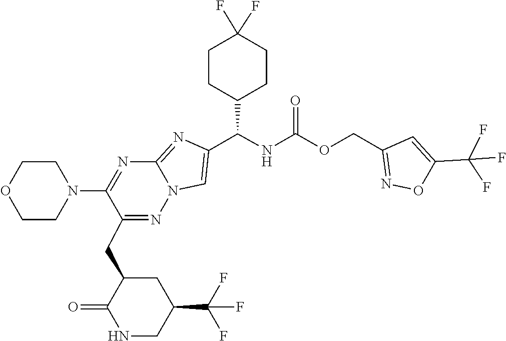 [SMILES: O=C(N[C@H](c1cn2nc(C[C@H]3C[C@@H](C(F)(F)F)CNC3=O)c(N3CCOCC3)nc2n1)C1CCC(F)(F)CC1)OCc1cc(C(F)(F)F)on1]
181 m/z = 725.2 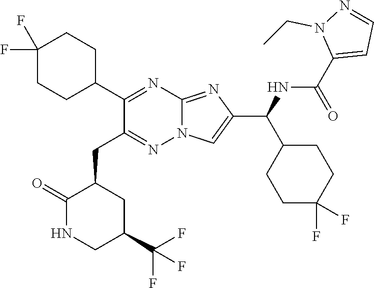 [SMILES: CCn1nccc1C(=O)N[C@H](c1cn2nc(C[C@H]3C[C@@H](C(F)(F)F)CNC3=O)c(C3CCC(F)(F)CC3)nc2n1)C1CCC(F)(F)CC1]
182 m/z = 687.3 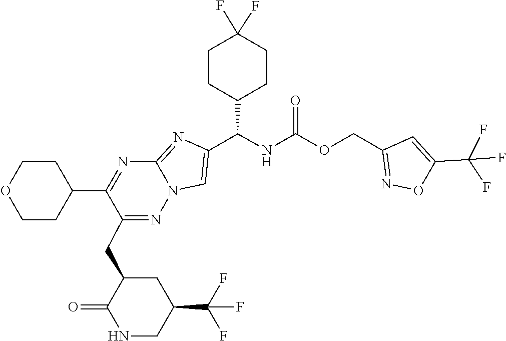 [SMILES: O=C(N[C@H](c1cn2nc(C[C@H]3C[C@@H](C(F)(F)F)CNC3=O)c(C3CCOCC3)nc2n1)C1CCC(F)(F)CC1)OCc1cc(C(F)(F)F)on1]
183 m/z = 724.2 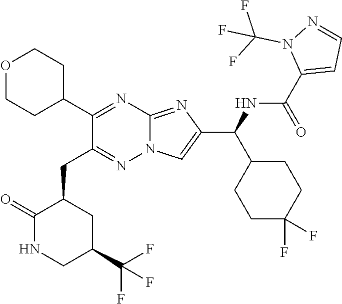 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1C(F)(F)F]
184 m/z = 693.3 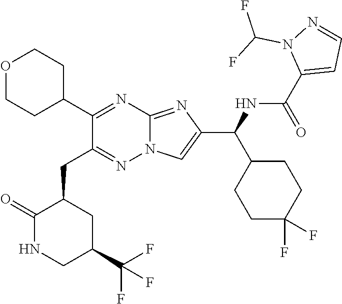 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1C(F)F]
185 m/z = 675.3 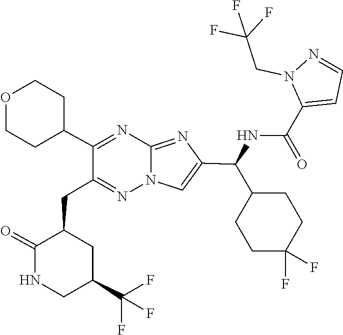 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1CC(F)(F)F]
186 m/z = 707.3 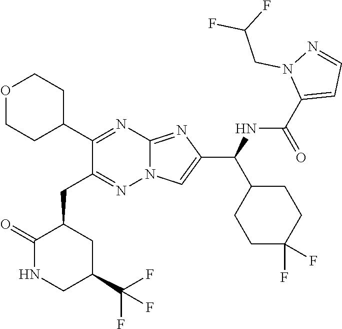 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1CC(F)F]
187 m/z = 689.3 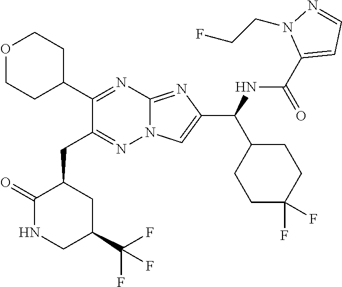 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1CCF]
188 m/z = 671.2 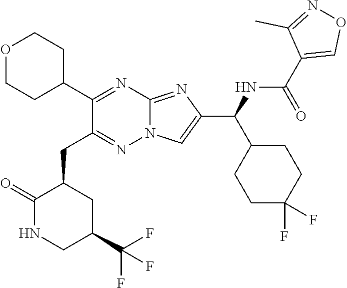 [SMILES: Cc1nocc1C(=O)N[C@H](c1cn2nc(C[C@H]3C[C@@H](C(F)(F)F)CNC3=O)c(C3CCOCC3)nc2n1)C1CCC(F)(F)CC1]
189 m/z = 640.2 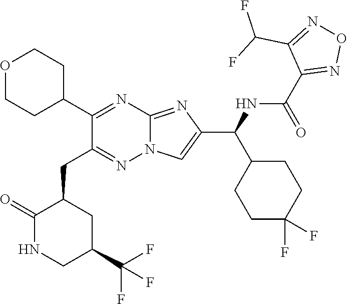 [SMILES: O=C(N[C@H](c1cn2nc(C[C@H]3C[C@@H](C(F)(F)F)CNC3=O)c(C3CCOCC3)nc2n1)C1CCC(F)(F)CC1)c1nonc1C(F)F]
190 m/z = 677.4 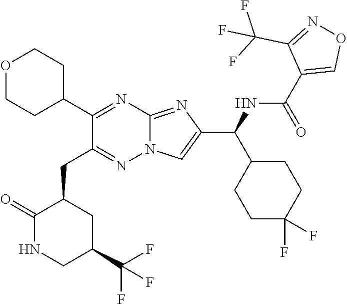 [SMILES: O=C(N[C@H](c1cn2nc(C[C@H]3C[C@@H](C(F)(F)F)CNC3=O)c(C3CCOCC3)nc2n1)C1CCC(F)(F)CC1)c1conc1C(F)(F)F]
191 m/z = 694.3 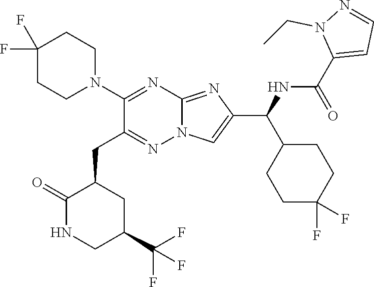 [SMILES: CCn1nccc1C(=O)N[C@H](c1cn2nc(C[C@H]3C[C@@H](C(F)(F)F)CNC3=O)c(N3CCC(F)(F)CC3)nc2n1)C1CCC(F)(F)CC1]
192 m/z = 688.3 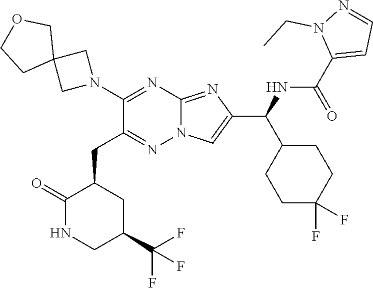 [SMILES: CCn1nccc1C(=O)N[C@H](c1cn2nc(C[C@H]3C[C@@H](C(F)(F)F)CNC3=O)c(N3CC4(CCOC4)C3)nc2n1)C1CCC(F)(F)CC1]
193 m/z = 680.3 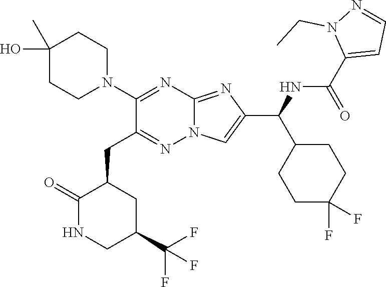 [SMILES: CCn1nccc1C(=O)N[C@H](c1cn2nc(C[C@H]3C[C@@H](C(F)(F)F)CNC3=O)c(N3CCC(C)(O)CC3)nc2n1)C1CCC(F)(F)CC1]
194 m/z = 682.3 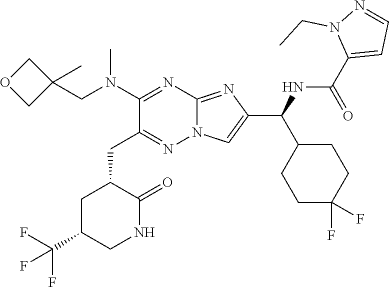 [SMILES: CCn1nccc1C(=O)N[C@H](c1cn2nc(C[C@H]3C[C@@H](C(F)(F)F)CNC3=O)c(N(C)CC3(C)COC3)nc2n1)C1CCC(F)(F)CC1]
195 m/z = 682.3 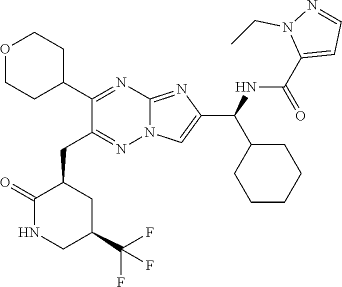 [SMILES: CCn1nccc1C(=O)N[C@H](c1cn2nc(C[C@H]3C[C@@H](C(F)(F)F)CNC3=O)c(C3CCOCC3)nc2n1)C1CCCCC1]
196 m/z = 617.3 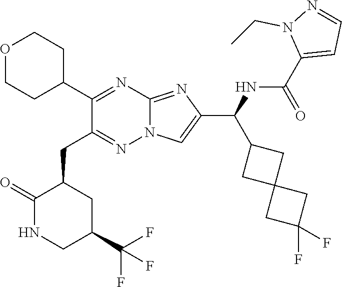 [SMILES: CCn1nccc1C(=O)N[C@H](c1cn2nc(C[C@H]3C[C@@H](C(F)(F)F)CNC3=O)c(C3CCOCC3)nc2n1)C1CC2(C1)CC(F)(F)C2]
197 m/z = 665.4 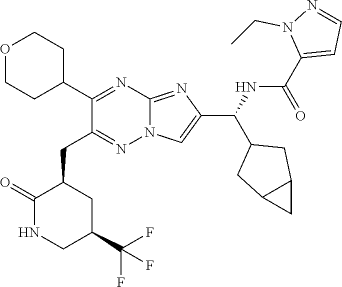 [SMILES: CCn1nccc1C(=O)N[C@@H](c1cn2nc(C[C@H]3C[C@@H](C(F)(F)F)CNC3=O)c(C3CCOCC3)nc2n1)C1CC2CC2C1]
198 m/z = 615.4 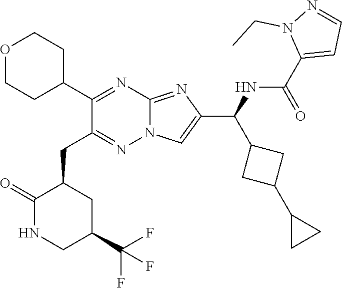 [SMILES: CCn1nccc1C(=O)N[C@H](c1cn2nc(C[C@H]3C[C@@H](C(F)(F)F)CNC3=O)c(C3CCOCC3)nc2n1)C1CC(C2CC2)C1]
199 m/z = 629.4 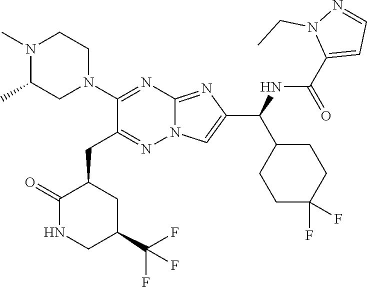 [SMILES: CCn1nccc1C(=O)N[C@H](c1cn2nc(C[C@H]3C[C@@H](C(F)(F)F)CNC3=O)c(N3CCN(C)[C@@H](C)C3)nc2n1)C1CCC(F)(F)CC1]
200 m/z = 681.4 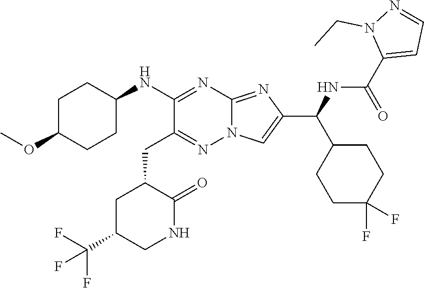 [SMILES: CCn1nccc1C(=O)N[C@H](c1cn2nc(C[C@H]3C[C@@H](C(F)(F)F)CNC3=O)c(N[C@H]3CC[C@@H](OC)CC3)nc2n1)C1CCC(F)(F)CC1]
201 m/z = 696.3 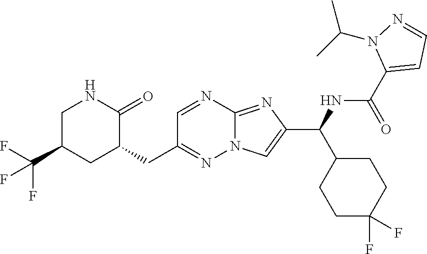 [SMILES: CC(C)n1nccc1C(=O)N[C@H](c1cn2nc(C[C@@H]3C[C@@H](C(F)(F)F)CNC3=O)cnc2n1)C1CCC(F)(F)CC1]
202 m/z = 583.3 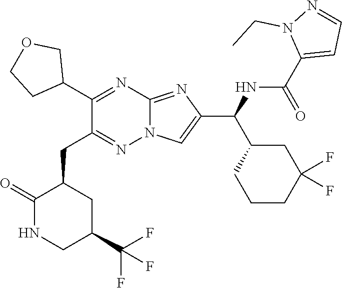 [SMILES: CCn1nccc1C(=O)N[C@H](c1cn2nc(C[C@H]3C[C@@H](C(F)(F)F)CNC3=O)c(C3CCOC3)nc2n1)[C@H]1CCCC(F)(F)C1]
203 m/z = 639.2 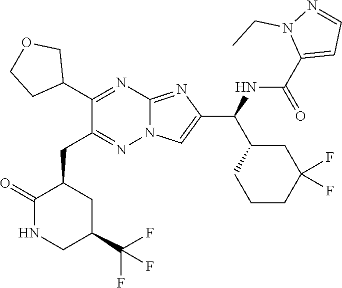 [SMILES: CCn1nccc1C(=O)N[C@H](c1cn2nc(C[C@H]3C[C@@H](C(F)(F)F)CNC3=O)c(C3CCOC3)nc2n1)[C@H]1CCCC(F)(F)C1]
204 m/z = 639.3 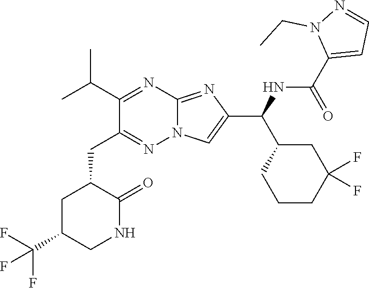 [SMILES: CCn1nccc1C(=O)N[C@H](c1cn2nc(C[C@H]3C[C@@H](C(F)(F)F)CNC3=O)c(C(C)C)nc2n1)[C@H]1CCCC(F)(F)C1]
205 m/z = 611.3 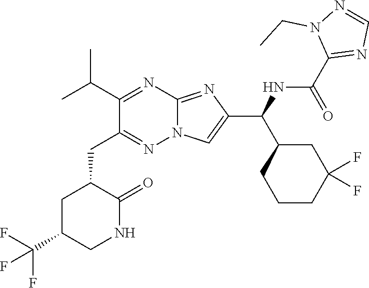 [SMILES: CCn1ncnc1C(=O)N[C@H](c1cn2nc(C[C@H]3C[C@@H](C(F)(F)F)CNC3=O)c(C(C)C)nc2n1)[C@@H]1CCCC(F)(F)C1]
206 m/z = 611.4 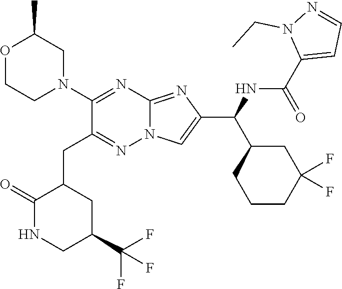 [SMILES: CCn1nccc1C(=O)N[C@H](c1cn2nc(CC3C[C@@H](C(F)(F)F)CNC3=O)c(N3CCO[C@@H](C)C3)nc2n1)[C@@H]1CCCC(F)(F)C1]
207 m/z = 668.4 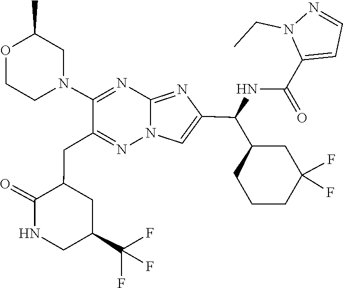 [SMILES: CCn1nccc1C(=O)N[C@H](c1cn2nc(CC3C[C@@H](C(F)(F)F)CNC3=O)c(N3CCO[C@@H](C)C3)nc2n1)[C@@H]1CCCC(F)(F)C1]
208 m/z = 668.4 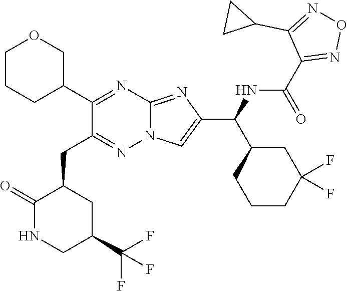 [SMILES: O=C(N[C@H](c1cn2nc(C[C@H]3C[C@@H](C(F)(F)F)CNC3=O)c(C3CCCOC3)nc2n1)[C@@H]1CCCC(F)(F)C1)c1nonc1C1CC1]
209 m/z = 667.4 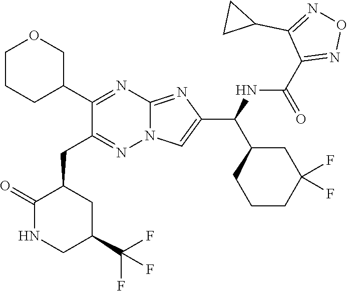 [SMILES: O=C(N[C@H](c1cn2nc(C[C@H]3C[C@@H](C(F)(F)F)CNC3=O)c(C3CCCOC3)nc2n1)[C@@H]1CCCC(F)(F)C1)c1nonc1C1CC1]
210 m/z = 667.4 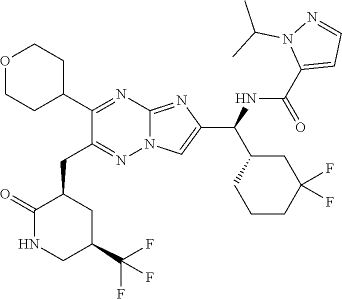 [SMILES: CC(C)n1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]
211 m/z = 667.4 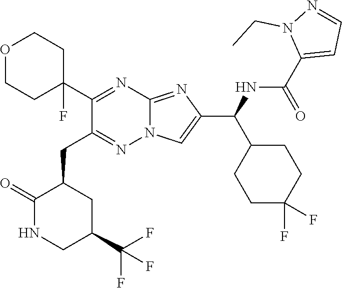 [SMILES: CCn1nccc1C(=O)N[C@H](c1cn2nc(C[C@H]3C[C@@H](C(F)(F)F)CNC3=O)c(C3(F)CCOCC3)nc2n1)C1CCC(F)(F)CC1]
212 m/z = 671.3 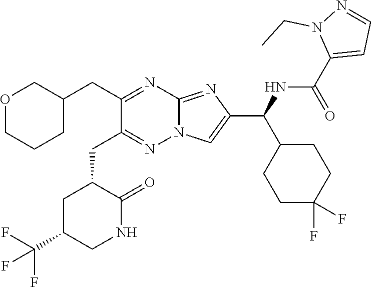 [SMILES: CCn1nccc1C(=O)N[C@H](c1cn2nc(C[C@H]3C[C@@H](C(F)(F)F)CNC3=O)c(CC3CCCOC3)nc2n1)C1CCC(F)(F)CC1]
213 m/z = 667.4 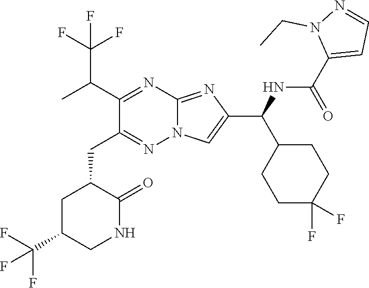 [SMILES: CCn1nccc1C(=O)N[C@H](c1cn2nc(C[C@H]3C[C@@H](C(F)(F)F)CNC3=O)c(C(C)C(F)(F)F)nc2n1)C1CCC(F)(F)CC1]
214 m/z = 665.4 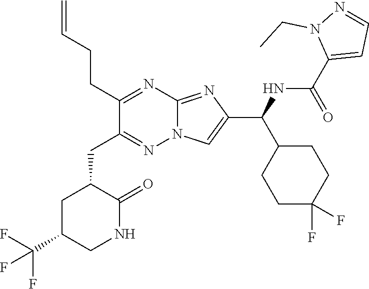 [SMILES: C=CCCc1nc2nc([C@@H](NC(=O)c3ccnn3CC)C3CCC(F)(F)CC3)cn2nc1C[C@H]1C[C@@H](C(F)(F)F)CNC1=O]
215 m/z = 623.5 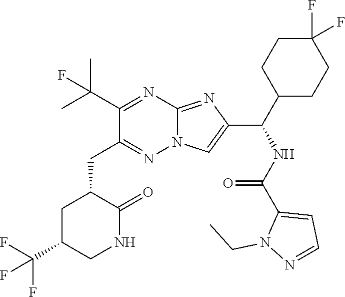 [SMILES: CCn1nccc1C(=O)N[C@H](c1cn2nc(C[C@H]3C[C@@H](C(F)(F)F)CNC3=O)c(C(C)(C)F)nc2n1)C1CCC(F)(F)CC1]
216 m/z = 629.2 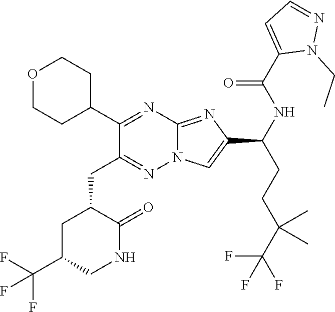 [SMILES: CCn1nccc1C(=O)N[C@@H](CCC(C)(C)C(F)(F)F)c1cn2nc(C[C@H]3C[C@@H](C(F)(F)F)CNC3=O)c(C3CCOCC3)nc2n1]
217 m/z = 617.3 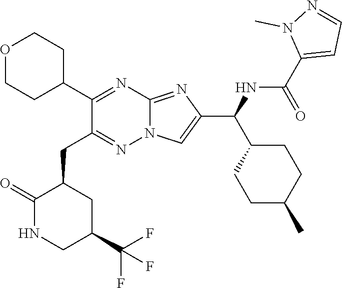 [SMILES: Cn1nccc1C(=O)N[C@H](c1cn2nc(C[C@H]3C[C@@H](C(F)(F)F)CNC3=O)c(C3CCOCC3)nc2n1)[C@H]1CC[C@H](C)CC1]
218 m/z = 617.3 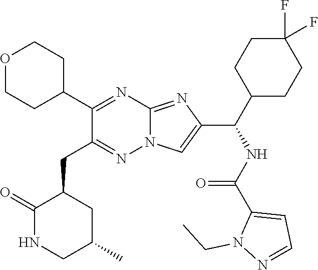 [SMILES: CCn1nccc1C(=O)N[C@H](c1cn2nc(C[C@H]3C[C@H](C)CNC3=O)c(C3CCOCC3)nc2n1)C1CCC(F)(F)CC1]
219 m/z = 599.3 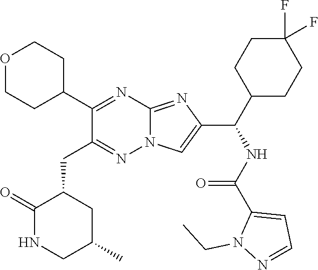 [SMILES: CCn1nccc1C(=O)N[C@H](c1cn2nc(C[C@@H]3C[C@H](C)CNC3=O)c(C3CCOCC3)nc2n1)C1CCC(F)(F)CC1]
220 m/z = 599.3 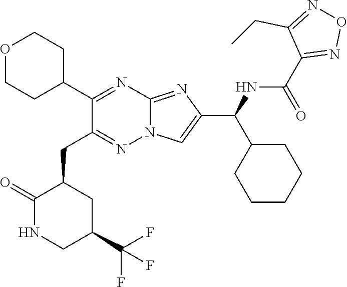 [SMILES: CCc1nonc1C(=O)N[C@H](c1cn2nc(C[C@H]3C[C@@H](C(F)(F)F)CNC3=O)c(C3CCOCC3)nc2n1)C1CCCCC1]
221 m/z = 619.4 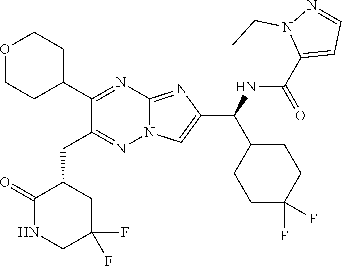 [SMILES: CCn1nccc1C(=O)N[C@H](c1cn2nc(C[C@@H]3CC(F)(F)CNC3=O)c(C3CCOCC3)nc2n1)C1CCC(F)(F)CC1]
222 m/z = 621.4 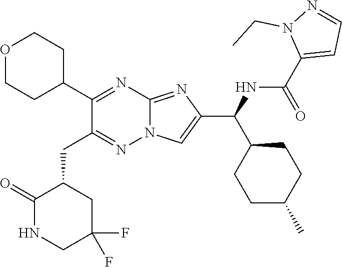 [SMILES: CCn1nccc1C(=O)N[C@H](c1cn2nc(C[C@@H]3CC(F)(F)CNC3=O)c(C3CCOCC3)nc2n1)[C@H]1CC[C@H](C)CC1]
223 m/z = 599.4 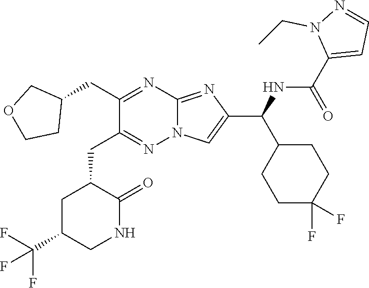 [SMILES: CCn1nccc1C(=O)N[C@H](c1cn2nc(C[C@H]3C[C@@H](C(F)(F)F)CNC3=O)c(C[C@@H]3CCOC3)nc2n1)C1CCC(F)(F)CC1]
224 m/z = 653.4 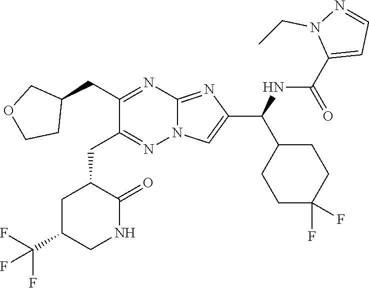 [SMILES: CCn1nccc1C(=O)N[C@H](c1cn2nc(C[C@H]3C[C@@H](C(F)(F)F)CNC3=O)c(C[C@H]3CCOC3)nc2n1)C1CCC(F)(F)CC1]
225 m/z = 653.4 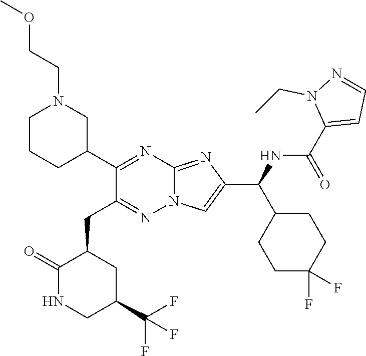 [SMILES: CCn1nccc1C(=O)N[C@H](c1cn2nc(C[C@H]3C[C@@H](C(F)(F)F)CNC3=O)c(C3CCCN(CCOC)C3)nc2n1)C1CCC(F)(F)CC1]
226 m/z = 710.3 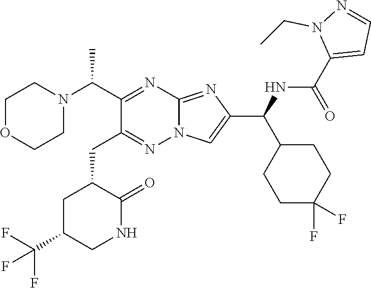 [SMILES: CCn1nccc1C(=O)N[C@H](c1cn2nc(C[C@H]3C[C@@H](C(F)(F)F)CNC3=O)c([C@@H](C)N3CCOCC3)nc2n1)C1CCC(F)(F)CC1]
227 m/z = 682.4 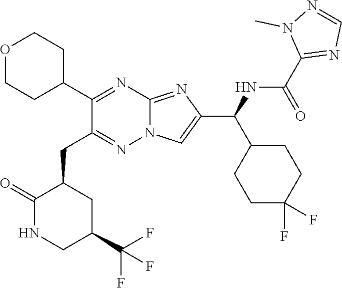 [SMILES: Cn1ncnc1C(=O)N[C@H](c1cn2nc(C[C@H]3C[C@@H](C(F)(F)F)CNC3=O)c(C3CCOCC3)nc2n1)C1CCC(F)(F)CC1]
228 m/z = 640.4 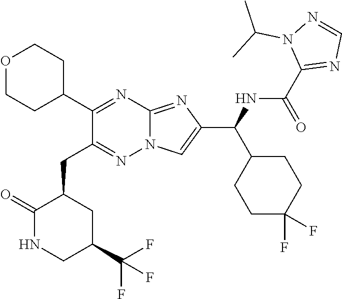 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]
229 m/z = 668.3 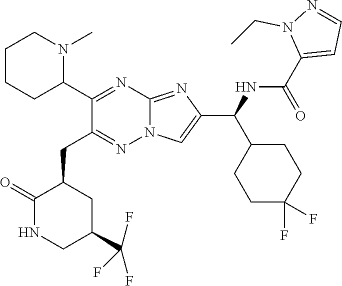 [SMILES: CCn1nccc1C(=O)N[C@H](c1cn2nc(C[C@H]3C[C@@H](C(F)(F)F)CNC3=O)c(C3CCCCN3C)nc2n1)C1CCC(F)(F)CC1]
230 m/z = 666.3 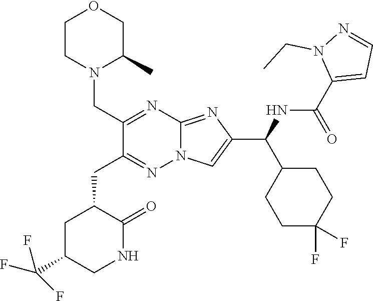 [SMILES: CCn1nccc1C(=O)N[C@H](c1cn2nc(C[C@H]3C[C@@H](C(F)(F)F)CNC3=O)c(CN3CCOC[C@H]3C)nc2n1)C1CCC(F)(F)CC1]
231 m/z = 682.6 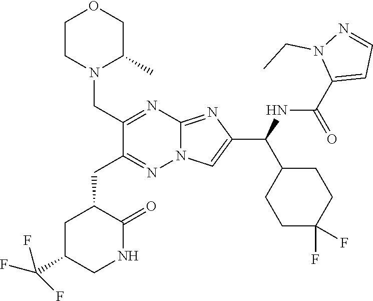 [SMILES: CCn1nccc1C(=O)N[C@H](c1cn2nc(C[C@H]3C[C@@H](C(F)(F)F)CNC3=O)c(CN3CCOC[C@@H]3C)nc2n1)C1CCC(F)(F)CC1]
232 m/z = 682.6 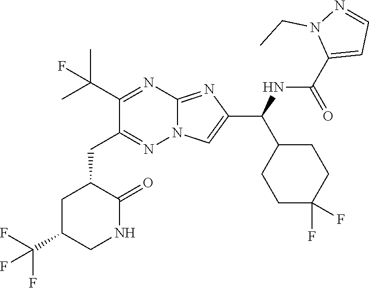 [SMILES: CCn1nccc1C(=O)N[C@H](c1cn2nc(C[C@H]3C[C@@H](C(F)(F)F)CNC3=O)c(C(C)(C)F)nc2n1)C1CCC(F)(F)CC1]
233 m/z = 629.3 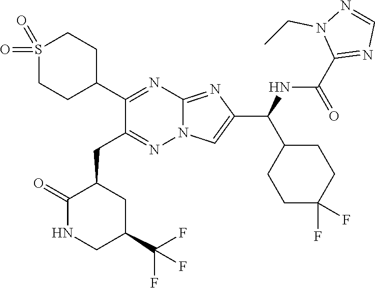 [SMILES: CCn1ncnc1C(=O)N[C@H](c1cn2nc(C[C@H]3C[C@@H](C(F)(F)F)CNC3=O)c(C3CCS(=O)(=O)CC3)nc2n1)C1CCC(F)(F)CC1]
234 m/z = 701.3 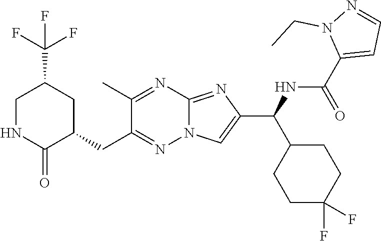 [SMILES: CCn1nccc1C(=O)N[C@H](c1cn2nc(C[C@H]3C[C@@H](C(F)(F)F)CNC3=O)c(C)nc2n1)C1CCC(F)(F)CC1]
235 m/z = 583.2 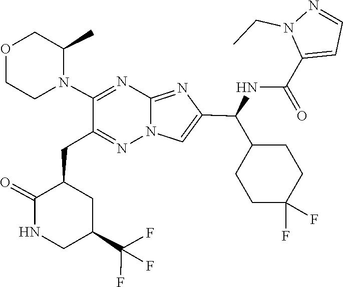 [SMILES: CCn1nccc1C(=O)N[C@H](c1cn2nc(C[C@H]3C[C@@H](C(F)(F)F)CNC3=O)c(N3CCOC[C@H]3C)nc2n1)C1CCC(F)(F)CC1]
236 m/z = 668.3 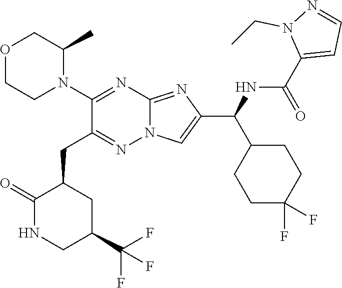 [SMILES: CCn1nccc1C(=O)N[C@H](c1cn2nc(C[C@H]3C[C@@H](C(F)(F)F)CNC3=O)c(N3CCOC[C@H]3C)nc2n1)C1CCC(F)(F)CC1]
237 m/z = 656.3 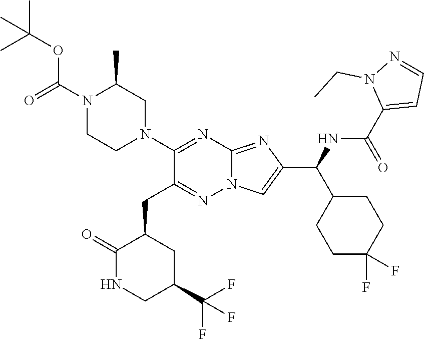 [SMILES: CCn1nccc1C(=O)N[C@H](c1cn2nc(C[C@H]3C[C@@H](C(F)(F)F)CNC3=O)c(N3CCN(C(=O)OC(C)(C)C)[C@@H](C)C3)nc2n1)C1CCC(F)(F)CC1]
238 m/z = 767.3 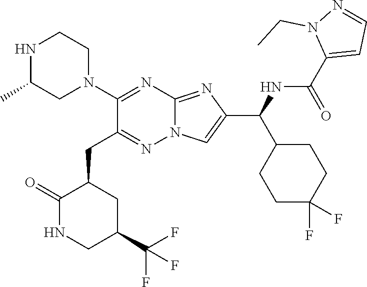 [SMILES: CCn1nccc1C(=O)N[C@H](c1cn2nc(C[C@H]3C[C@@H](C(F)(F)F)CNC3=O)c(N3CCN[C@@H](C)C3)nc2n1)C1CCC(F)(F)CC1]
239 m/z = 667.3 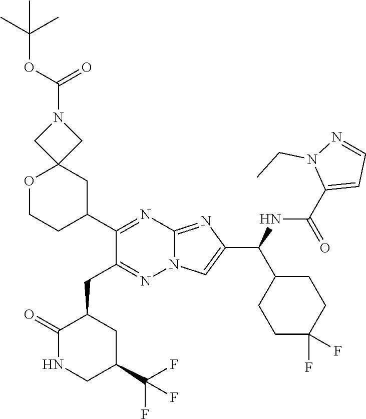 [SMILES: CCn1nccc1C(=O)N[C@H](c1cn2nc(C[C@H]3C[C@@H](C(F)(F)F)CNC3=O)c(C3CCOC4(C3)CN(C(=O)OC(C)(C)C)C4)nc2n1)C1CCC(F)(F)CC1]
240 m/z = 794.3 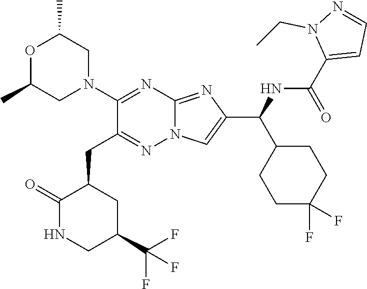 [SMILES: CCn1nccc1C(=O)N[C@H](c1cn2nc(C[C@H]3C[C@@H](C(F)(F)F)CNC3=O)c(N3C[C@@H](C)O[C@H](C)C3)nc2n1)C1CCC(F)(F)CC1]
241 m/z = 682.3 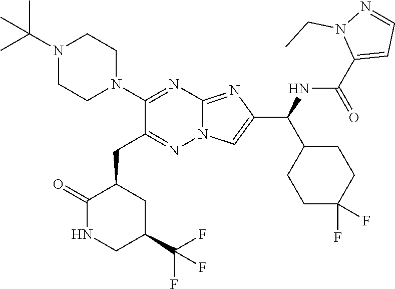 [SMILES: CCn1nccc1C(=O)N[C@H](c1cn2nc(C[C@H]3C[C@@H](C(F)(F)F)CNC3=O)c(N3CCN(C(C)(C)C)CC3)nc2n1)C1CCC(F)(F)CC1]
242 m/z = 709.3 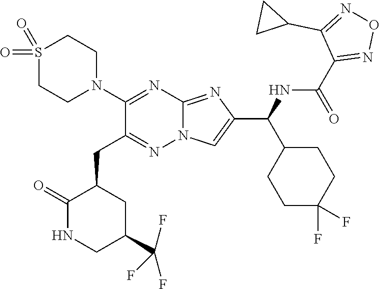 [SMILES: O=C(N[C@H](c1cn2nc(C[C@H]3C[C@@H](C(F)(F)F)CNC3=O)c(N3CCS(=O)(=O)CC3)nc2n1)C1CCC(F)(F)CC1)c1nonc1C1CC1]
243 m/z = 715.2 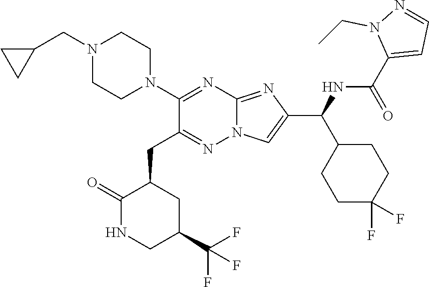 [SMILES: CCn1nccc1C(=O)N[C@H](c1cn2nc(C[C@H]3C[C@@H](C(F)(F)F)CNC3=O)c(N3CCN(CC4CC4)CC3)nc2n1)C1CCC(F)(F)CC1]
244 m/z = 707.3 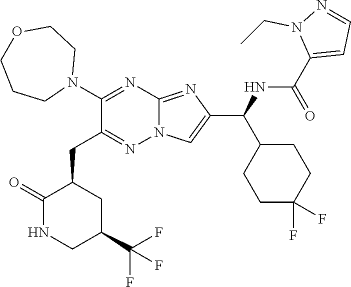 [SMILES: CCn1nccc1C(=O)N[C@H](c1cn2nc(C[C@H]3C[C@@H](C(F)(F)F)CNC3=O)c(N3CCCOCC3)nc2n1)C1CCC(F)(F)CC1]
245 m/z = 668.3 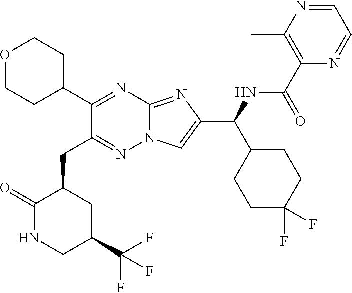 [SMILES: Cc1nccnc1C(=O)N[C@H](c1cn2nc(C[C@H]3C[C@@H](C(F)(F)F)CNC3=O)c(C3CCOCC3)nc2n1)C1CCC(F)(F)CC1]
246 m/z = 651.3 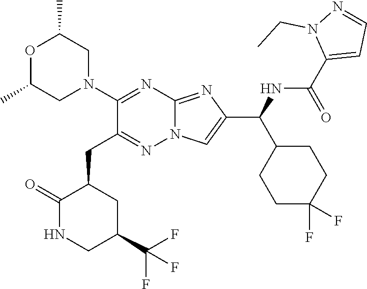 [SMILES: CCn1nccc1C(=O)N[C@H](c1cn2nc(C[C@H]3C[C@@H](C(F)(F)F)CNC3=O)c(N3C[C@@H](C)O[C@@H](C)C3)nc2n1)C1CCC(F)(F)CC1]
247 m/z = 682.3 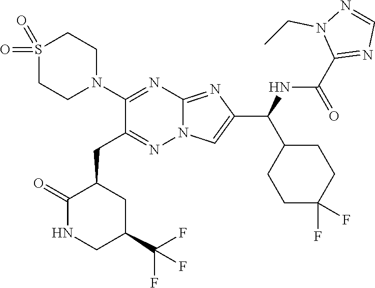 [SMILES: CCn1ncnc1C(=O)N[C@H](c1cn2nc(C[C@H]3C[C@@H](C(F)(F)F)CNC3=O)c(N3CCS(=O)(=O)CC3)nc2n1)C1CCC(F)(F)CC1]
248 m/z = 702.3 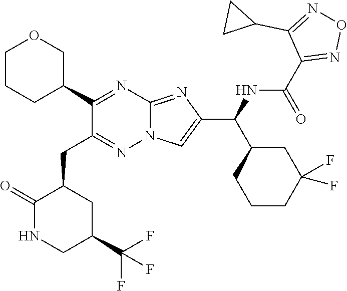 [SMILES: O=C(N[C@H](c1cn2nc(C[C@H]3C[C@@H](C(F)(F)F)CNC3=O)c([C@H]3CCCOC3)nc2n1)[C@@H]1CCCC(F)(F)C1)c1nonc1C1CC1]
249 m/z = 667.4 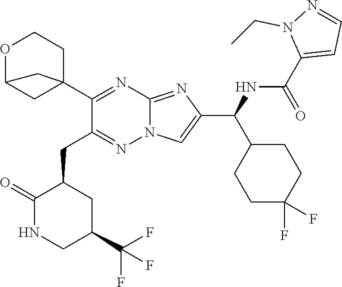 [SMILES: CCn1nccc1C(=O)N[C@H](c1cn2nc(C[C@H]3C[C@@H](C(F)(F)F)CNC3=O)c(C34CCOC(C3)C4)nc2n1)C1CCC(F)(F)CC1]
250 m/z = 665.3 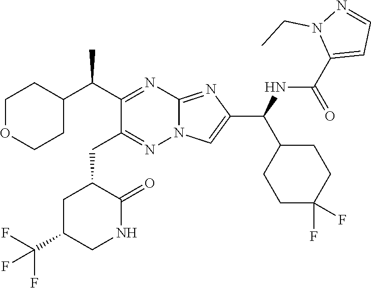 [SMILES: CCn1nccc1C(=O)N[C@H](c1cn2nc(C[C@H]3C[C@@H](C(F)(F)F)CNC3=O)c([C@H](C)C3CCOCC3)nc2n1)C1CCC(F)(F)CC1]
251 m/z = 681.3 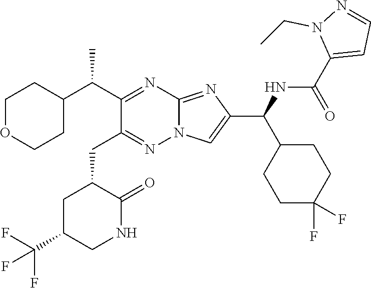 [SMILES: CCn1nccc1C(=O)N[C@H](c1cn2nc(C[C@H]3C[C@@H](C(F)(F)F)CNC3=O)c([C@@H](C)C3CCOCC3)nc2n1)C1CCC(F)(F)CC1]
252 m/z = 681.4 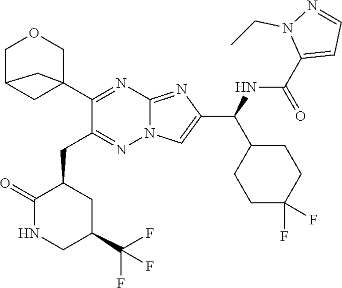 [SMILES: CCn1nccc1C(=O)N[C@H](c1cn2nc(C[C@H]3C[C@@H](C(F)(F)F)CNC3=O)c(C34COCC(C3)C4)nc2n1)C1CCC(F)(F)CC1]
253 m/z = 665.3 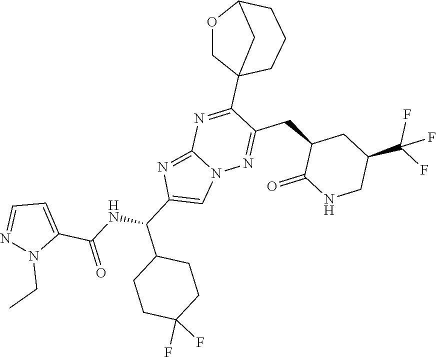 [SMILES: CCn1nccc1C(=O)N[C@H](c1cn2nc(C[C@H]3C[C@@H](C(F)(F)F)CNC3=O)c(C34CCCC(C3)OC4)nc2n1)C1CCC(F)(F)CC1]
254 m/z = 679.2 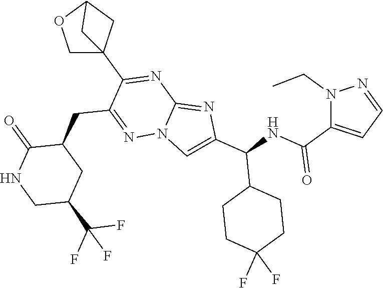 [SMILES: CCn1nccc1C(=O)N[C@H](c1cn2nc(C[C@H]3C[C@@H](C(F)(F)F)CNC3=O)c(C34COC(C3)C4)nc2n1)C1CCC(F)(F)CC1]
255 m/z = 651.3 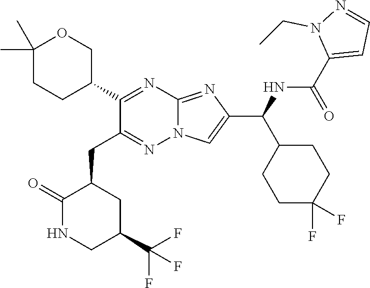 [SMILES: CCn1nccc1C(=O)N[C@H](c1cn2nc(C[C@H]3C[C@@H](C(F)(F)F)CNC3=O)c([C@@H]3CCC(C)(C)OC3)nc2n1)C1CCC(F)(F)CC1]
256 m/z = 681.4 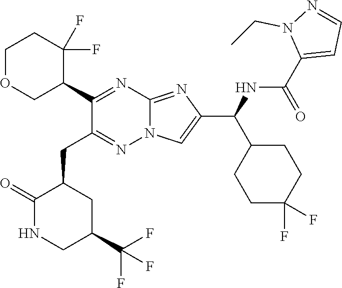 [SMILES: CCn1nccc1C(=O)N[C@H](c1cn2nc(C[C@H]3C[C@@H](C(F)(F)F)CNC3=O)c([C@H]3COCCC3(F)F)nc2n1)C1CCC(F)(F)CC1]
257 m/z = 689.3 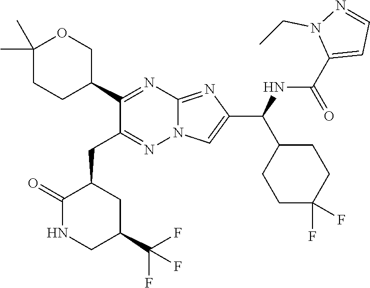 [SMILES: CCn1nccc1C(=O)N[C@H](c1cn2nc(C[C@H]3C[C@@H](C(F)(F)F)CNC3=O)c([C@H]3CCC(C)(C)OC3)nc2n1)C1CCC(F)(F)CC1]
258 m/z = 681.4 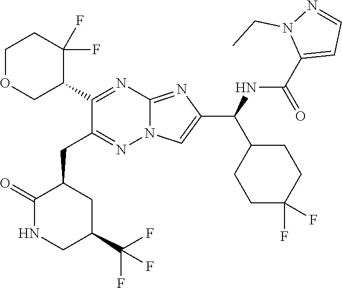 [SMILES: CCn1nccc1C(=O)N[C@H](c1cn2nc(C[C@H]3C[C@@H](C(F)(F)F)CNC3=O)c([C@@H]3COCCC3(F)F)nc2n1)C1CCC(F)(F)CC1]
259 m/z = 689.3 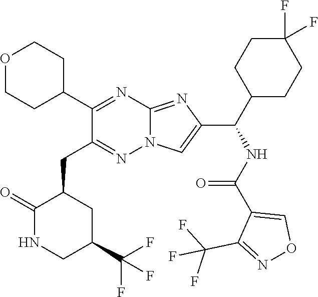 [SMILES: O=C(N[C@H](c1cn2nc(C[C@H]3C[C@@H](C(F)(F)F)CNC3=O)c(C3CCOCC3)nc2n1)C1CCC(F)(F)CC1)c1conc1C(F)(F)F]
260 m/z = 690.3 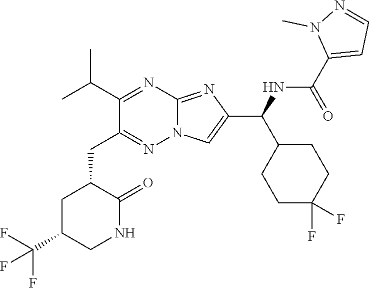 [SMILES: CC(C)c1nc2nc([C@@H](NC(=O)c3ccnn3C)C3CCC(F)(F)CC3)cn2nc1C[C@H]1C[C@@H](C(F)(F)F)CNC1=O]
261 m/z = 597.3 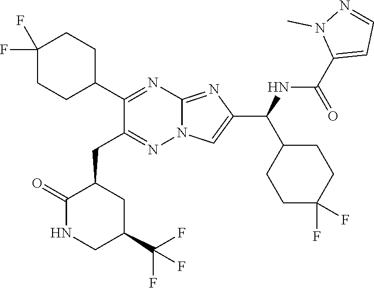 [SMILES: Cn1nccc1C(=O)N[C@H](c1cn2nc(C[C@H]3C[C@@H](C(F)(F)F)CNC3=O)c(C3CCC(F)(F)CC3)nc2n1)C1CCC(F)(F)CC1]
262 m/z = 673.3 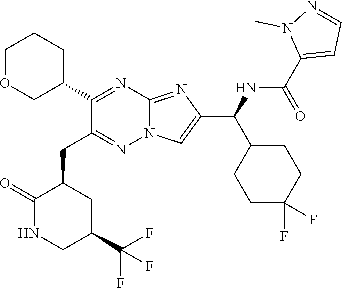 [SMILES: Cn1nccc1C(=O)N[C@H](c1cn2nc(C[C@H]3C[C@@H](C(F)(F)F)CNC3=O)c([C@H]3CCCOC3)nc2n1)C1CCC(F)(F)CC1]
263 m/z = 639.4 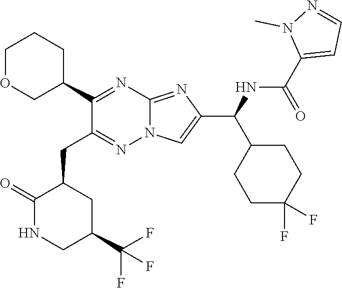 [SMILES: Cn1nccc1C(=O)N[C@H](c1cn2nc(C[C@H]3C[C@@H](C(F)(F)F)CNC3=O)c([C@@H]3CCCOC3)nc2n1)C1CCC(F)(F)CC1]
264 m/z = 639.2 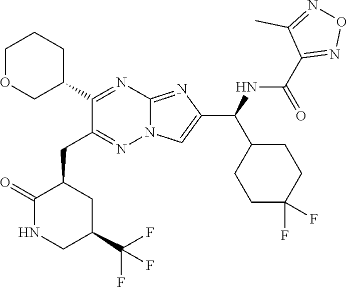 [SMILES: Cc1nonc1C(=O)N[C@H](c1cn2nc(C[C@H]3C[C@@H](C(F)(F)F)CNC3=O)c([C@H]3CCCOC3)nc2n1)C1CCC(F)(F)CC1]
265 m/z = 641.4 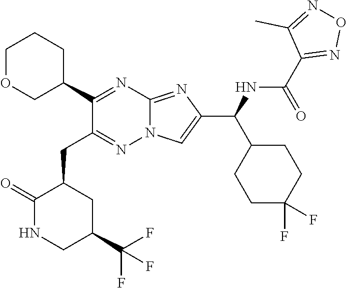 [SMILES: Cc1nonc1C(=O)N[C@H](c1cn2nc(C[C@H]3C[C@@H](C(F)(F)F)CNC3=O)c([C@@H]3CCCOC3)nc2n1)C1CCC(F)(F)CC1]
266 m/z = 641.4 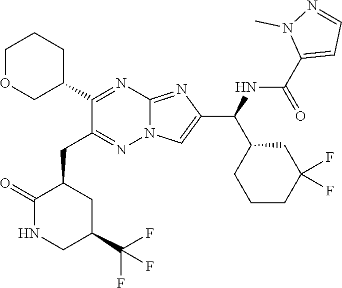 [SMILES: Cn1nccc1C(=O)N[C@H](c1cn2nc(C[C@H]3C[C@@H](C(F)(F)F)CNC3=O)c([C@H]3CCCOC3)nc2n1)[C@H]1CCCC(F)(F)C1]
267 m/z = 639.4 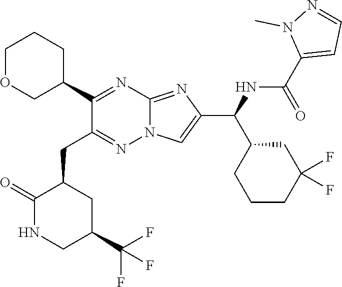 [SMILES: Cn1nccc1C(=O)N[C@H](c1cn2nc(C[C@H]3C[C@@H](C(F)(F)F)CNC3=O)c([C@@H]3CCCOC3)nc2n1)[C@H]1CCCC(F)(F)C1]
268 m/z = 639.4 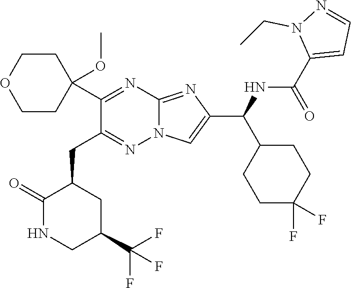 [SMILES: CCn1nccc1C(=O)N[C@H](c1cn2nc(C[C@H]3C[C@@H](C(F)(F)F)CNC3=O)c(C3(OC)CCOCC3)nc2n1)C1CCC(F)(F)CC1]
269 m/z = 683.3 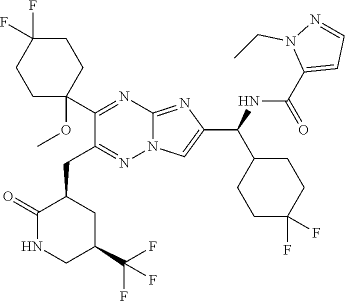 [SMILES: CCn1nccc1C(=O)N[C@H](c1cn2nc(C[C@H]3C[C@@H](C(F)(F)F)CNC3=O)c(C3(OC)CCC(F)(F)CC3)nc2n1)C1CCC(F)(F)CC1]
270 m/z = 717.4 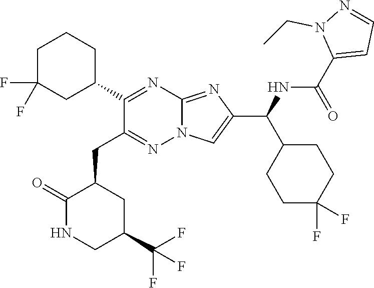 [SMILES: CCn1nccc1C(=O)N[C@H](c1cn2nc(C[C@H]3C[C@@H](C(F)(F)F)CNC3=O)c([C@H]3CCCC(F)(F)C3)nc2n1)C1CCC(F)(F)CC1]
271 m/z = 687.3 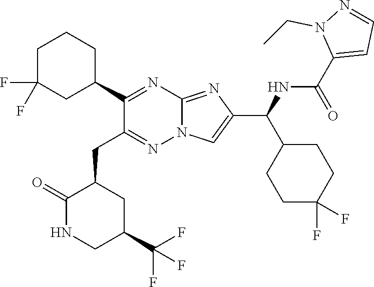 [SMILES: CCn1nccc1C(=O)N[C@H](c1cn2nc(C[C@H]3C[C@@H](C(F)(F)F)CNC3=O)c([C@@H]3CCCC(F)(F)C3)nc2n1)C1CCC(F)(F)CC1]
272 m/z = 687.3 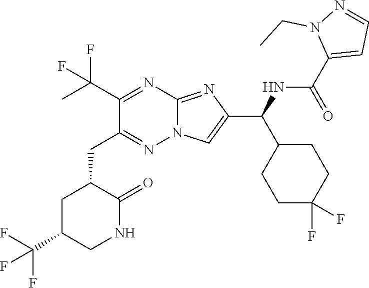 [SMILES: CCn1nccc1C(=O)N[C@H](c1cn2nc(C[C@H]3C[C@@H](C(F)(F)F)CNC3=O)c(C(C)(F)F)nc2n1)C1CCC(F)(F)CC1]
273 m/z = 633.2 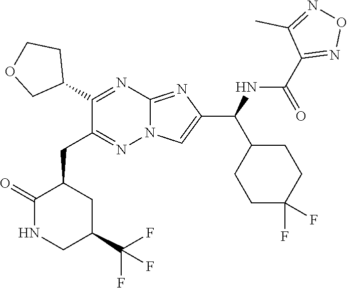 [SMILES: Cc1nonc1C(=O)N[C@H](c1cn2nc(C[C@H]3C[C@@H](C(F)(F)F)CNC3=O)c([C@H]3CCOC3)nc2n1)C1CCC(F)(F)CC1]
274 m/z = 627.3 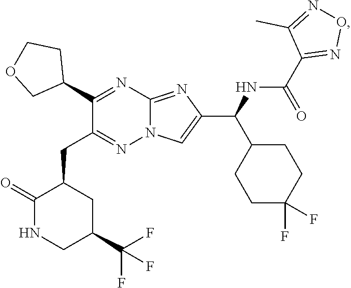 [SMILES: Cc1nonc1C(=O)N[C@H](c1cn2nc(C[C@H]3C[C@@H](C(F)(F)F)CNC3=O)c([C@@H]3CCOC3)nc2n1)C1CCC(F)(F)CC1]
275 m/z = 627.3 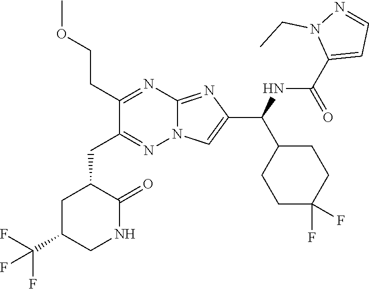 [SMILES: CCn1nccc1C(=O)N[C@H](c1cn2nc(C[C@H]3C[C@@H](C(F)(F)F)CNC3=O)c(CCOC)nc2n1)C1CCC(F)(F)CC1]
276 m/z = 627.4 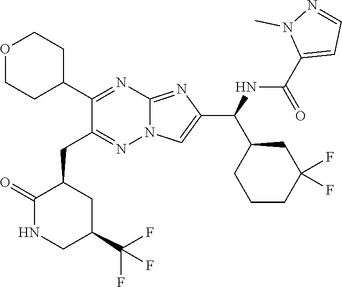 [SMILES: Cn1nccc1C(=O)N[C@H](c1cn2nc(C[C@H]3C[C@@H](C(F)(F)F)CNC3=O)c(C3CCOCC3)nc2n1)[C@@H]1CCCC(F)(F)C1]
277 m/z = 639.4 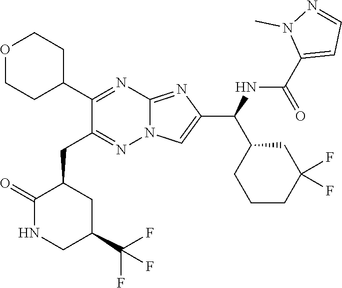 [SMILES: Cn1nccc1C(=O)N[C@H](c1cn2nc(C[C@H]3C[C@@H](C(F)(F)F)CNC3=O)c(C3CCOCC3)nc2n1)[C@H]1CCCC(F)(F)C1]
278 m/z = 639.4 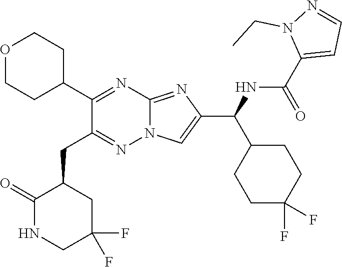 [SMILES: CCn1nccc1C(=O)N[C@H](c1cn2nc(C[C@H]3CC(F)(F)CNC3=O)c(C3CCOCC3)nc2n1)C1CCC(F)(F)CC1]
279 m/z = 621.4 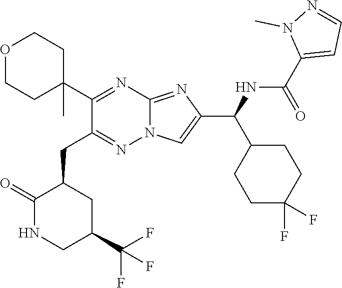 [SMILES: Cn1nccc1C(=O)N[C@H](c1cn2nc(C[C@H]3C[C@@H](C(F)(F)F)CNC3=O)c(C3(C)CCOCC3)nc2n1)C1CCC(F)(F)CC1]
280 m/z = 653.4 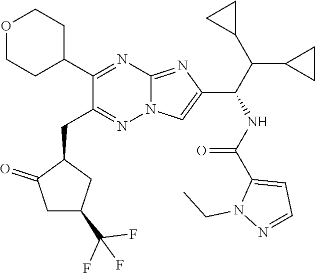 [SMILES: CCn1nccc1C(=O)N[C@H](c1cn2nc(C[C@H]3C[C@@H](C(F)(F)F)CC3=O)c(C3CCOCC3)nc2n1)C(C1CC1)C1CC1]
281 m/z = 615.4 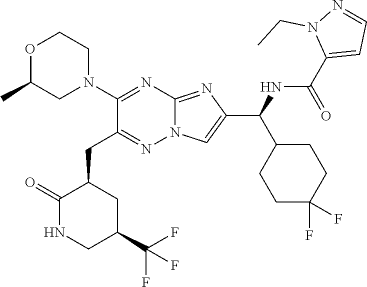 [SMILES: CCn1nccc1C(=O)N[C@H](c1cn2nc(C[C@H]3C[C@@H](C(F)(F)F)CNC3=O)c(N3CCO[C@H](C)C3)nc2n1)C1CCC(F)(F)CC1]
282 m/z = 668.4 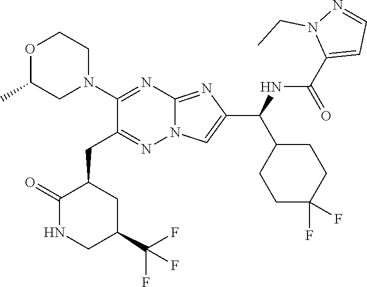 [SMILES: CCn1nccc1C(=O)N[C@H](c1cn2nc(C[C@H]3C[C@@H](C(F)(F)F)CNC3=O)c(N3CCO[C@@H](C)C3)nc2n1)C1CCC(F)(F)CC1]
283 m/z = 668.4 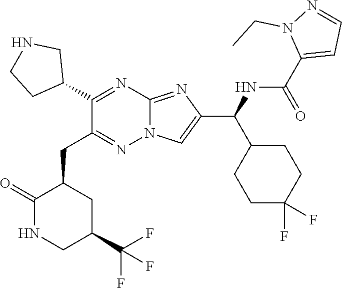 [SMILES: CCn1nccc1C(=O)N[C@H](c1cn2nc(C[C@H]3C[C@@H](C(F)(F)F)CNC3=O)c([C@@H]3CCNC3)nc2n1)C1CCC(F)(F)CC1]
284 m/z = 638.4 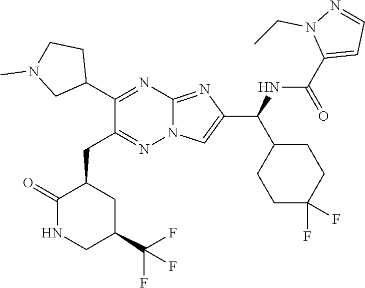 [SMILES: CCn1nccc1C(=O)N[C@H](c1cn2nc(C[C@H]3C[C@@H](C(F)(F)F)CNC3=O)c(C3CCN(C)C3)nc2n1)C1CCC(F)(F)CC1]
285 m/z = 652.4 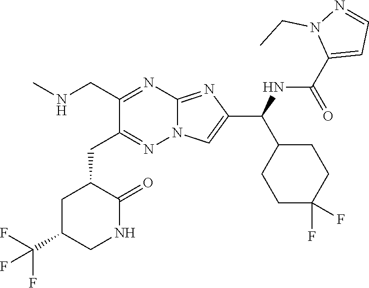 [SMILES: CCn1nccc1C(=O)N[C@H](c1cn2nc(C[C@H]3C[C@@H](C(F)(F)F)CNC3=O)c(CNC)nc2n1)C1CCC(F)(F)CC1]
286 m/z = 612.4 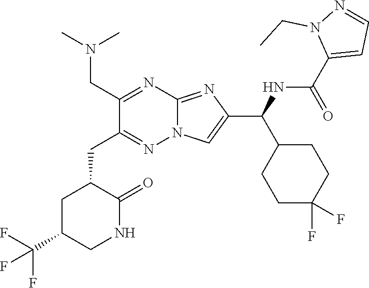 [SMILES: CCn1nccc1C(=O)N[C@H](c1cn2nc(C[C@H]3C[C@@H](C(F)(F)F)CNC3=O)c(CN(C)C)nc2n1)C1CCC(F)(F)CC1]
287 m/z = 626.3 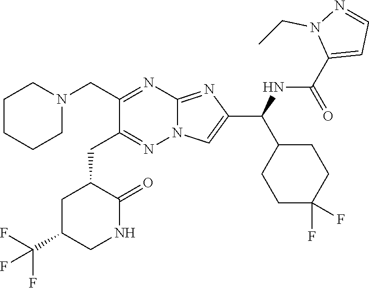 [SMILES: CCn1nccc1C(=O)N[C@H](c1cn2nc(C[C@H]3C[C@@H](C(F)(F)F)CNC3=O)c(CN3CCCCC3)nc2n1)C1CCC(F)(F)CC1]
288 m/z = 666.4 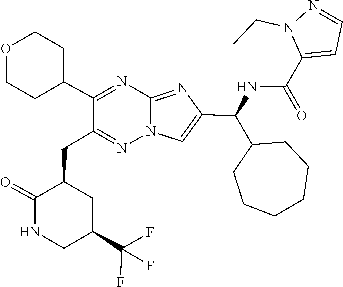 [SMILES: CCn1nccc1C(=O)N[C@H](c1cn2nc(C[C@H]3C[C@@H](C(F)(F)F)CNC3=O)c(C3CCOCC3)nc2n1)C1CCCCCC1]
289 m/z = 631.4 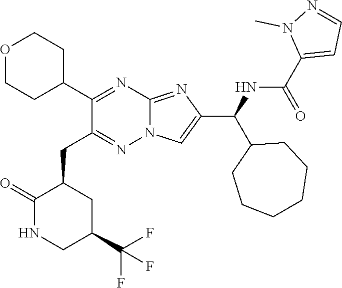 [SMILES: Cn1nccc1C(=O)N[C@H](c1cn2nc(C[C@H]3C[C@@H](C(F)(F)F)CNC3=O)c(C3CCOCC3)nc2n1)C1CCCCCC1]
290 m/z = 617.4 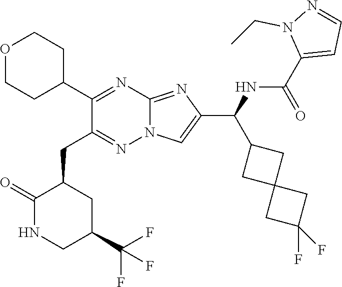 [SMILES: CCn1nccc1C(=O)N[C@H](c1cn2nc(C[C@H]3C[C@@H](C(F)(F)F)CNC3=O)c(C3CCOCC3)nc2n1)C1CC2(C1)CC(F)(F)C2]
291 m/z = 665.4 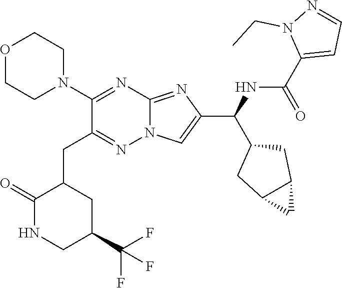 [SMILES: CCn1nccc1C(=O)N[C@H](c1cn2nc(CC3C[C@@H](C(F)(F)F)CNC3=O)c(N3CCOCC3)nc2n1)[C@H]1C[C@H]2C[C@H]2C1]
292 m/z = 615.4 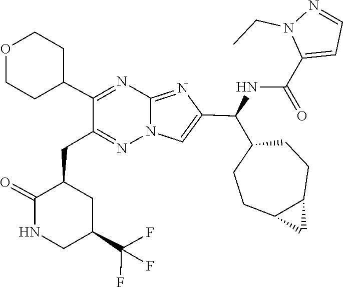 [SMILES: CCn1nccc1C(=O)N[C@H](c1cn2nc(C[C@H]3C[C@@H](C(F)(F)F)CNC3=O)c(C3CCOCC3)nc2n1)[C@@H]1CC[C@@H]2C[C@@H]2CC1]
293 m/z = 643.5 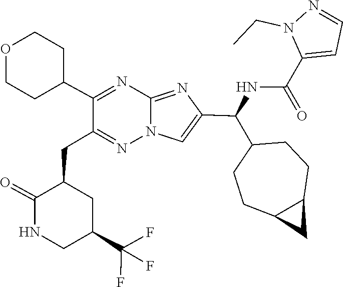 [SMILES: CCn1nccc1C(=O)N[C@H](c1cn2nc(C[C@H]3C[C@@H](C(F)(F)F)CNC3=O)c(C3CCOCC3)nc2n1)C1CC[C@@H]2C[C@@H]2CC1]
294 m/z = 643.5 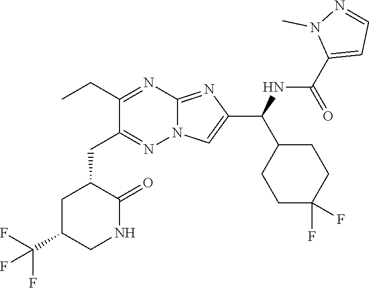 [SMILES: CCc1nc2nc([C@@H](NC(=O)c3ccnn3C)C3CCC(F)(F)CC3)cn2nc1C[C@H]1C[C@@H](C(F)(F)F)CNC1=O]
295 m/z = 583.4 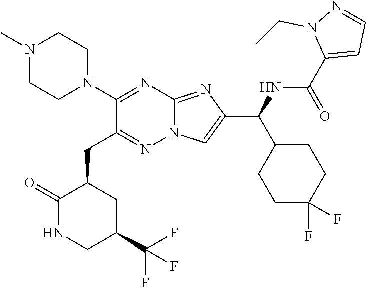 [SMILES: CCn1nccc1C(=O)N[C@H](c1cn2nc(C[C@H]3C[C@@H](C(F)(F)F)CNC3=O)c(N3CCN(C)CC3)nc2n1)C1CCC(F)(F)CC1]
296 m/z = 667.5 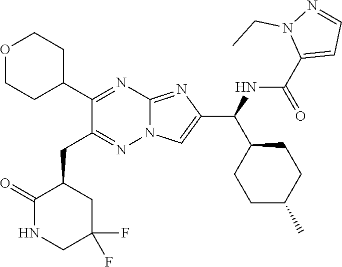 [SMILES: CCn1nccc1C(=O)N[C@H](c1cn2nc(C[C@H]3CC(F)(F)CNC3=O)c(C3CCOCC3)nc2n1)[C@H]1CC[C@H](C)CC1]
297 m/z = 599.4 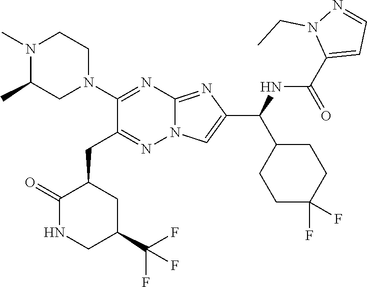 [SMILES: CCn1nccc1C(=O)N[C@H](c1cn2nc(C[C@H]3C[C@@H](C(F)(F)F)CNC3=O)c(N3CCN(C)[C@H](C)C3)nc2n1)C1CCC(F)(F)CC1]
298 m/z = 681.4 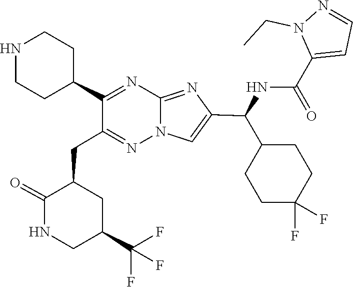
299 m/z = 652.4 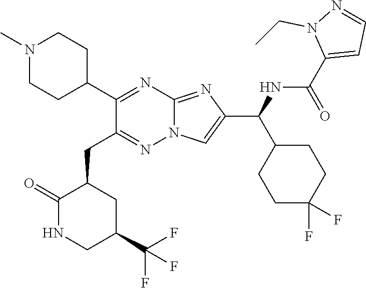 [SMILES: CCn1nccc1C(=O)N[C@H](c1cn2nc(C[C@H]3C[C@@H](C(F)(F)F)CNC3=O)c(C3CCN(C)CC3)nc2n1)C1CCC(F)(F)CC1]
300 m/z = 666.4 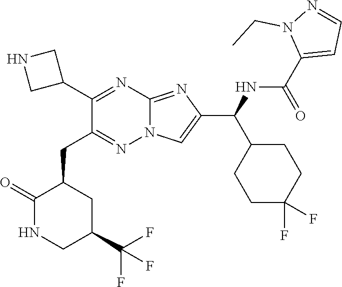 [SMILES: CCn1nccc1C(=O)N[C@H](c1cn2nc(C[C@H]3C[C@@H](C(F)(F)F)CNC3=O)c(C3CNC3)nc2n1)C1CCC(F)(F)CC1]
301 m/z = 624.3 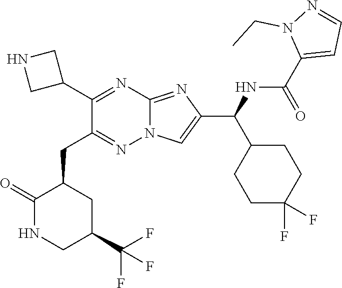 [SMILES: CCn1nccc1C(=O)N[C@H](c1cn2nc(C[C@H]3C[C@@H](C(F)(F)F)CNC3=O)c(C3CNC3)nc2n1)C1CCC(F)(F)CC1]
302 m/z = 638.3 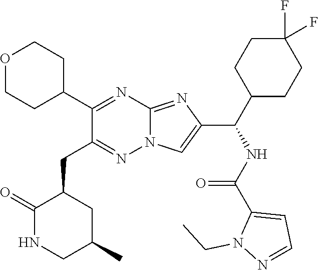 [SMILES: CCn1nccc1C(=O)N[C@H](c1cn2nc(C[C@H]3C[C@@H](C)CNC3=O)c(C3CCOCC3)nc2n1)C1CCC(F)(F)CC1]
303 m/z = 599.5 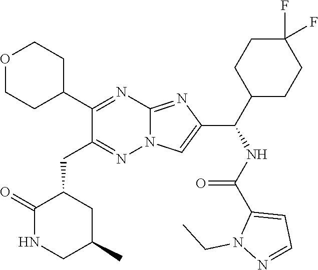 [SMILES: CCn1nccc1C(=O)N[C@H](c1cn2nc(C[C@@H]3C[C@@H](C)CNC3=O)c(C3CCOCC3)nc2n1)C1CCC(F)(F)CC1]
304 m/z = 599.5 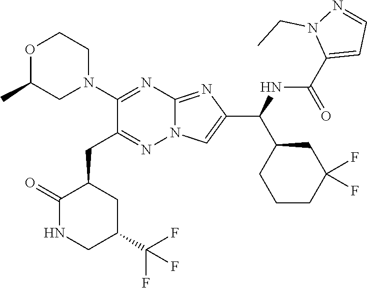 [SMILES: CCn1nccc1C(=O)N[C@H](c1cn2nc(C[C@H]3C[C@H](C(F)(F)F)CNC3=O)c(N3CCO[C@H](C)C3)nc2n1)[C@@H]1CCCC(F)(F)C1]
305 m/z = 668.4 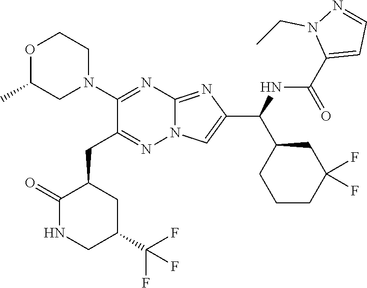 [SMILES: CCn1nccc1C(=O)N[C@H](c1cn2nc(C[C@H]3C[C@H](C(F)(F)F)CNC3=O)c(N3CCO[C@@H](C)C3)nc2n1)[C@@H]1CCCC(F)(F)C1]
306 m/z = 668.4 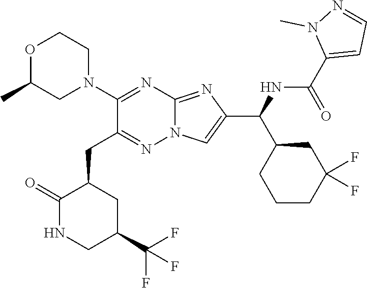 [SMILES: C[C@@H]1CN(c2nc3nc([C@@H](NC(=O)c4ccnn4C)[C@@H]4CCCC(F)(F)C4)cn3nc2C[C@H]2C[C@@H](C(F)(F)F)CNC2=O)CCO1]
307 m/z = 654.4 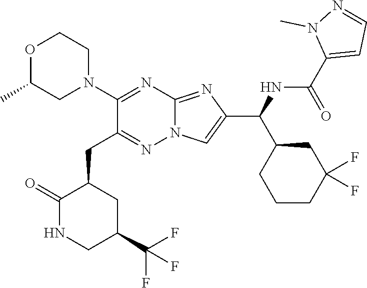 [SMILES: C[C@H]1CN(c2nc3nc([C@@H](NC(=O)c4ccnn4C)[C@@H]4CCCC(F)(F)C4)cn3nc2C[C@H]2C[C@@H](C(F)(F)F)CNC2=O)CCO1]
308 m/z = 654.4 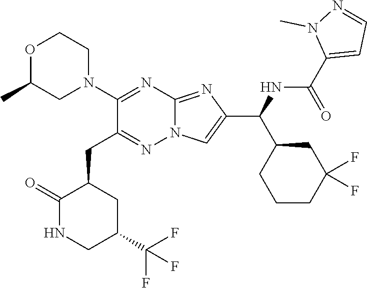 [SMILES: C[C@@H]1CN(c2nc3nc([C@@H](NC(=O)c4ccnn4C)[C@@H]4CCCC(F)(F)C4)cn3nc2C[C@H]2C[C@H](C(F)(F)F)CNC2=O)CCO1]
309 m/z = 654.4 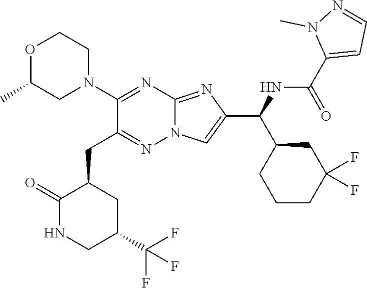 [SMILES: C[C@H]1CN(c2nc3nc([C@@H](NC(=O)c4ccnn4C)[C@@H]4CCCC(F)(F)C4)cn3nc2C[C@H]2C[C@H](C(F)(F)F)CNC2=O)CCO1]
310 m/z = 654.4 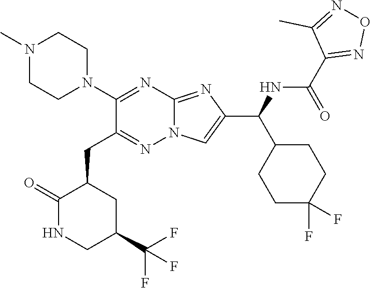 [SMILES: Cc1nonc1C(=O)N[C@H](c1cn2nc(C[C@H]3C[C@@H](C(F)(F)F)CNC3=O)c(N3CCN(C)CC3)nc2n1)C1CCC(F)(F)CC1]
311 m/z = 655.3 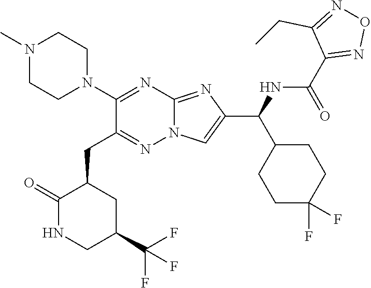 [SMILES: CCc1nonc1C(=O)N[C@H](c1cn2nc(C[C@H]3C[C@@H](C(F)(F)F)CNC3=O)c(N3CCN(C)CC3)nc2n1)C1CCC(F)(F)CC1]
312 m/z = 669.3 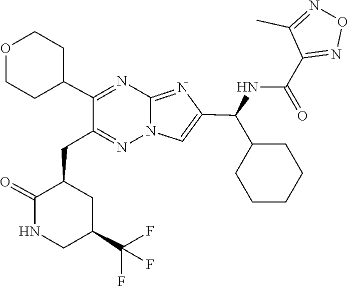 [SMILES: Cc1nonc1C(=O)N[C@H](c1cn2nc(C[C@H]3C[C@@H](C(F)(F)F)CNC3=O)c(C3CCOCC3)nc2n1)C1CCCCC1]
313 m/z = 605.4 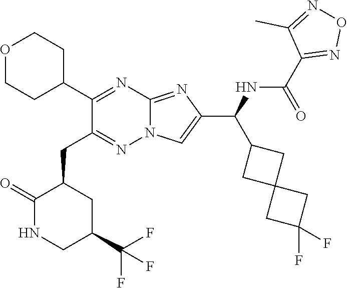 [SMILES: Cc1nonc1C(=O)N[C@H](c1cn2nc(C[C@H]3C[C@@H](C(F)(F)F)CNC3=O)c(C3CCOCC3)nc2n1)C1CC2(C1)CC(F)(F)C2]
314 m/z = 653.3 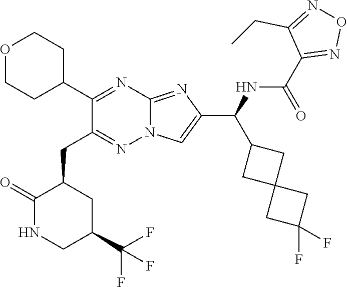 [SMILES: CCc1nonc1C(=O)N[C@H](c1cn2nc(C[C@H]3C[C@@H](C(F)(F)F)CNC3=O)c(C3CCOCC3)nc2n1)C1CC2(C1)CC(F)(F)C2]
315 m/z = 667.3 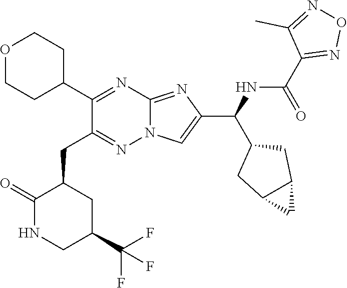 [SMILES: Cc1nonc1C(=O)N[C@H](c1cn2nc(C[C@H]3C[C@@H](C(F)(F)F)CNC3=O)c(C3CCOCC3)nc2n1)[C@H]1C[C@H]2C[C@H]2C1]
316 m/z = 603.3 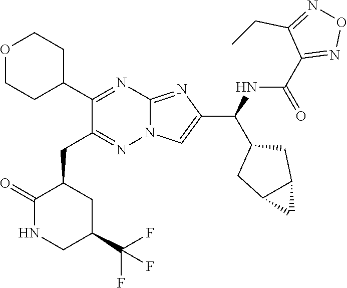 [SMILES: CCc1nonc1C(=O)N[C@H](c1cn2nc(C[C@H]3C[C@@H](C(F)(F)F)CNC3=O)c(C3CCOCC3)nc2n1)[C@H]1C[C@H]2C[C@H]2C1]
317 m/z = 617.3 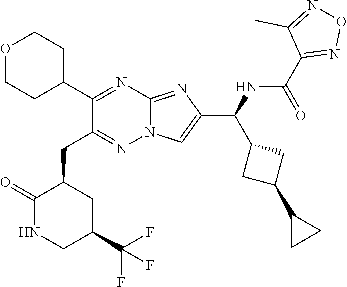 [SMILES: Cc1nonc1C(=O)N[C@H](c1cn2nc(C[C@H]3C[C@@H](C(F)(F)F)CNC3=O)c(C3CCOCC3)nc2n1)[C@H]1C[C@H](C2CC2)C1]
318 m/z = 617.3 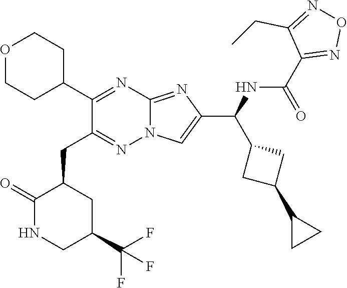 [SMILES: CCc1nonc1C(=O)N[C@H](c1cn2nc(C[C@H]3C[C@@H](C(F)(F)F)CNC3=O)c(C3CCOCC3)nc2n1)[C@H]1C[C@H](C2CC2)C1]
319 m/z = 631.4 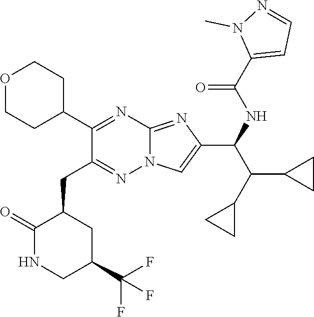 [SMILES: Cn1nccc1C(=O)N[C@H](c1cn2nc(C[C@H]3C[C@@H](C(F)(F)F)CNC3=O)c(C3CCOCC3)nc2n1)C(C1CC1)C1CC1]
320 m/z = 615.4 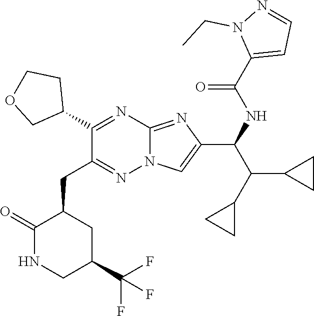 [SMILES: CCn1nccc1C(=O)N[C@H](c1cn2nc(C[C@H]3C[C@@H](C(F)(F)F)CNC3=O)c([C@H]3CCOC3)nc2n1)C(C1CC1)C1CC1]
321 m/z = 615.4 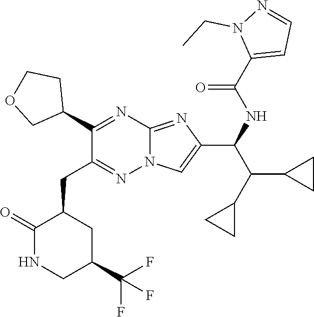 [SMILES: CCn1nccc1C(=O)N[C@H](c1cn2nc(C[C@H]3C[C@@H](C(F)(F)F)CNC3=O)c([C@@H]3CCOC3)nc2n1)C(C1CC1)C1CC1]
322 m/z = 615.4 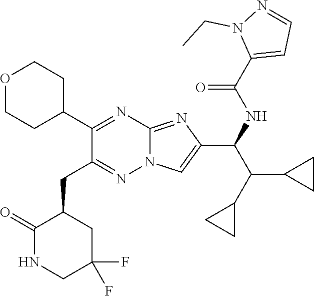 [SMILES: CCn1nccc1C(=O)N[C@H](c1cn2nc(C[C@H]3CC(F)(F)CNC3=O)c(C3CCOCC3)nc2n1)C(C1CC1)C1CC1]
323 m/z = 597.3 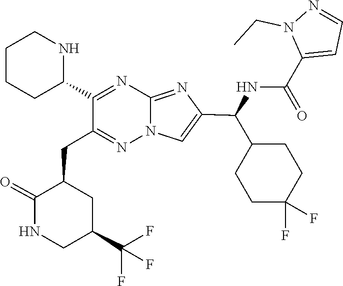 [SMILES: CCn1nccc1C(=O)N[C@H](c1cn2nc(C[C@H]3C[C@@H](C(F)(F)F)CNC3=O)c([C@@H]3CCCCN3)nc2n1)C1CCC(F)(F)CC1]
324 m/z = 652.4 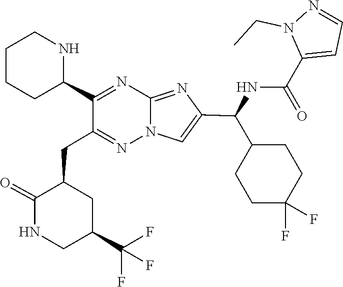 [SMILES: CCn1nccc1C(=O)N[C@H](c1cn2nc(C[C@H]3C[C@@H](C(F)(F)F)CNC3=O)c([C@H]3CCCCN3)nc2n1)C1CCC(F)(F)CC1]
325 m/z = 652.4 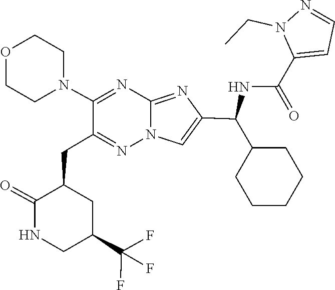 [SMILES: CCn1nccc1C(=O)N[C@H](c1cn2nc(C[C@H]3C[C@@H](C(F)(F)F)CNC3=O)c(N3CCOCC3)nc2n1)C1CCCCC1]
326 m/z = 618.4 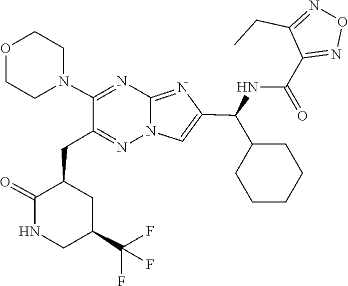 [SMILES: CCc1nonc1C(=O)N[C@H](c1cn2nc(C[C@H]3C[C@@H](C(F)(F)F)CNC3=O)c(N3CCOCC3)nc2n1)C1CCCCC1]
327 m/z = 620.4 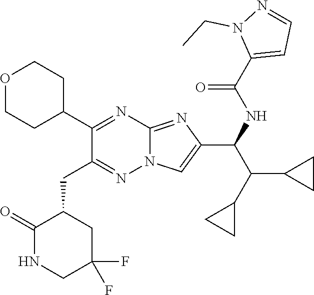 [SMILES: CCn1nccc1C(=O)N[C@H](c1cn2nc(C[C@@H]3CC(F)(F)CNC3=O)c(C3CCOCC3)nc2n1)C(C1CC1)C1CC1]
328 m/z = 597.3 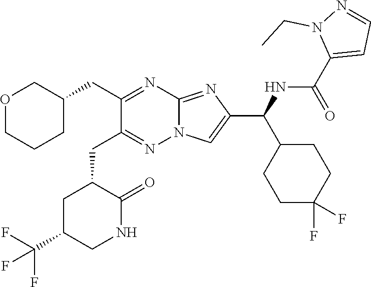 [SMILES: CCn1nccc1C(=O)N[C@H](c1cn2nc(C[C@H]3C[C@@H](C(F)(F)F)CNC3=O)c(C[C@@H]3CCCOC3)nc2n1)C1CCC(F)(F)CC1]
329 m/z = 667.4 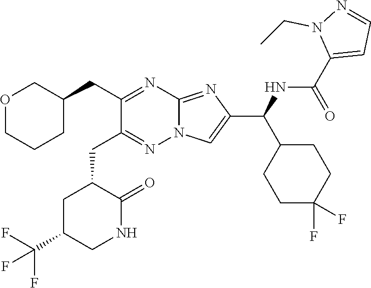 [SMILES: CCn1nccc1C(=O)N[C@H](c1cn2nc(C[C@H]3C[C@@H](C(F)(F)F)CNC3=O)c(C[C@H]3CCCOC3)nc2n1)C1CCC(F)(F)CC1]
330 m/z = 667.4 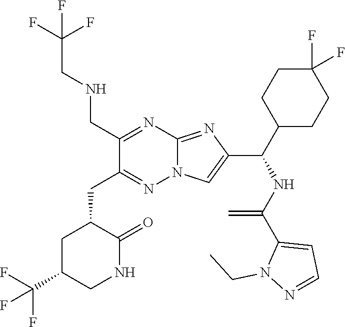 [SMILES: C=C(N[C@H](c1cn2nc(C[C@H]3C[C@@H](C(F)(F)F)CNC3=O)c(CNCC(F)(F)F)nc2n1)C1CCC(F)(F)CC1)c1ccnn1CC]
331 m/z = 680.3 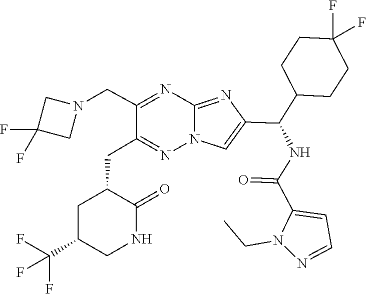 [SMILES: CCn1nccc1C(=O)N[C@H](c1cn2nc(C[C@H]3C[C@@H](C(F)(F)F)CNC3=O)c(CN3CC(F)(F)C3)nc2n1)C1CCC(F)(F)CC1]
332 m/z = 674.3 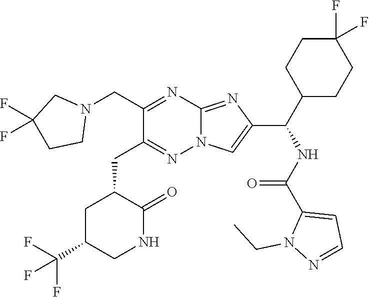 [SMILES: CCn1nccc1C(=O)N[C@H](c1cn2nc(C[C@H]3C[C@@H](C(F)(F)F)CNC3=O)c(CN3CCC(F)(F)C3)nc2n1)C1CCC(F)(F)CC1]
333 m/z = 688.3 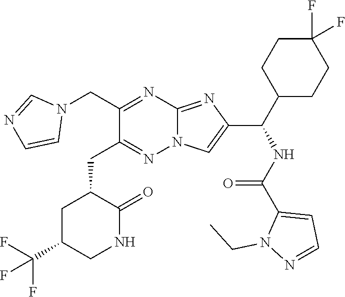 [SMILES: CCn1nccc1C(=O)N[C@H](c1cn2nc(C[C@H]3C[C@@H](C(F)(F)F)CNC3=O)c(Cn3ccnc3)nc2n1)C1CCC(F)(F)CC1]
334 m/z = 649.4 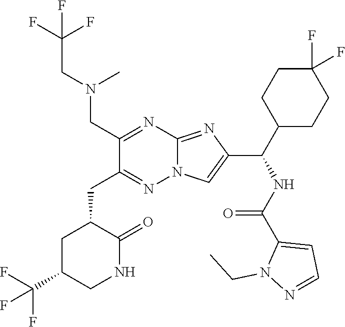 [SMILES: CCn1nccc1C(=O)N[C@H](c1cn2nc(C[C@H]3C[C@@H](C(F)(F)F)CNC3=O)c(CN(C)CC(F)(F)F)nc2n1)C1CCC(F)(F)CC1]
335 m/z = 694.3 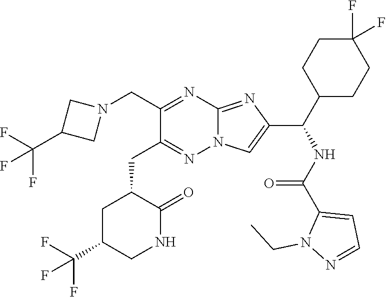 [SMILES: CCn1nccc1C(=O)N[C@H](c1cn2nc(C[C@H]3C[C@@H](C(F)(F)F)CNC3=O)c(CN3CC(C(F)(F)F)C3)nc2n1)C1CCC(F)(F)CC1]
336 m/z = 706.2 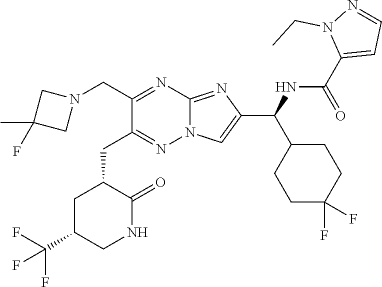 [SMILES: CCn1nccc1C(=O)N[C@H](c1cn2nc(C[C@H]3C[C@@H](C(F)(F)F)CNC3=O)c(CN3CC(C)(F)C3)nc2n1)C1CCC(F)(F)CC1]
337 m/z = 670.4 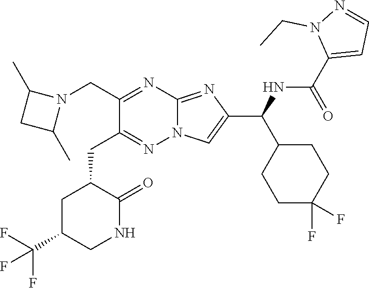 [SMILES: CCn1nccc1C(=O)N[C@H](c1cn2nc(C[C@H]3C[C@@H](C(F)(F)F)CNC3=O)c(CN3C(C)CC3C)nc2n1)C1CCC(F)(F)CC1]
338 m/z = 666.4 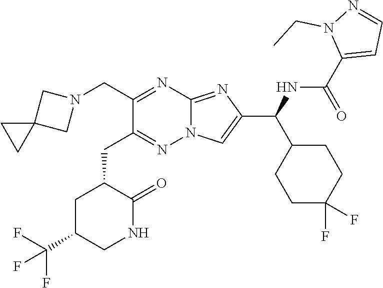 [SMILES: CCn1nccc1C(=O)N[C@H](c1cn2nc(C[C@H]3C[C@@H](C(F)(F)F)CNC3=O)c(CN3CC4(CC4)C3)nc2n1)C1CCC(F)(F)CC1]
339 m/z = 664.4 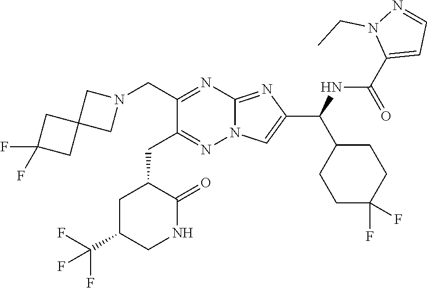 [SMILES: CCn1nccc1C(=O)N[C@H](c1cn2nc(C[C@H]3C[C@@H](C(F)(F)F)CNC3=O)c(CN3CC4(C3)CC(F)(F)C4)nc2n1)C1CCC(F)(F)CC1]
340 m/z = 714.3 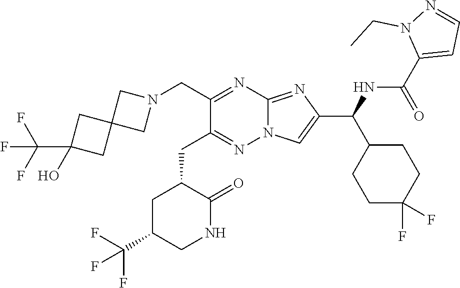 [SMILES: CCn1nccc1C(=O)N[C@H](c1cn2nc(C[C@H]3C[C@@H](C(F)(F)F)CNC3=O)c(CN3CC4(C3)CC(O)(C(F)(F)F)C4)nc2n1)C1CCC(F)(F)CC1]
341 m/z = 762.4 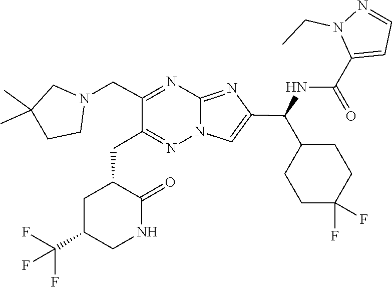 [SMILES: CCn1nccc1C(=O)N[C@H](c1cn2nc(C[C@H]3C[C@@H](C(F)(F)F)CNC3=O)c(CN3CCC(C)(C)C3)nc2n1)C1CCC(F)(F)CC1]
342 m/z = 680.5 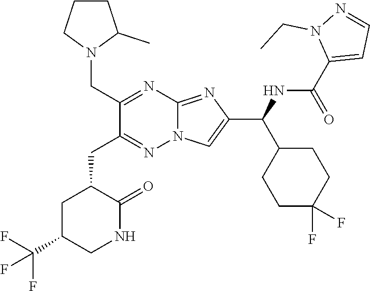 [SMILES: CCn1nccc1C(=O)N[C@H](c1cn2nc(C[C@H]3C[C@@H](C(F)(F)F)CNC3=O)c(CN3CCCC3C)nc2n1)C1CCC(F)(F)CC1]
343 m/z = 666.5 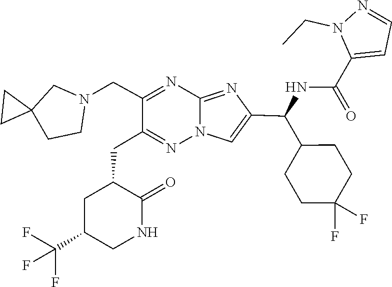 [SMILES: CCn1nccc1C(=O)N[C@H](c1cn2nc(C[C@H]3C[C@@H](C(F)(F)F)CNC3=O)c(CN3CCC4(CC4)C3)nc2n1)C1CCC(F)(F)CC1]
344 m/z = 678.3 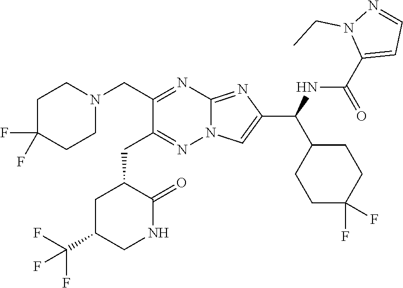 [SMILES: CCn1nccc1C(=O)N[C@H](c1cn2nc(C[C@H]3C[C@@H](C(F)(F)F)CNC3=O)c(CN3CCC(F)(F)CC3)nc2n1)C1CCC(F)(F)CC1]
345 m/z = 702.3 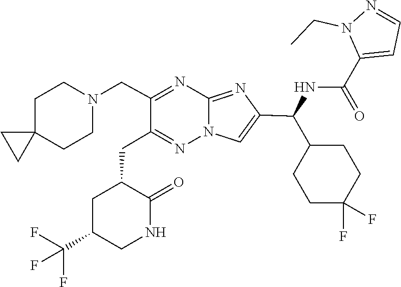 [SMILES: CCn1nccc1C(=O)N[C@H](c1cn2nc(C[C@H]3C[C@@H](C(F)(F)F)CNC3=O)c(CN3CCC4(CC3)CC4)nc2n1)C1CCC(F)(F)CC1]
346 m/z = 692.4 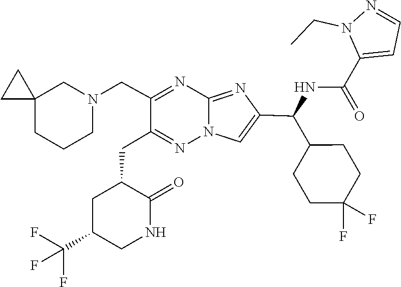 [SMILES: CCn1nccc1C(=O)N[C@H](c1cn2nc(C[C@H]3C[C@@H](C(F)(F)F)CNC3=O)c(CN3CCCC4(CC4)C3)nc2n1)C1CCC(F)(F)CC1]
347 m/z = 692.4 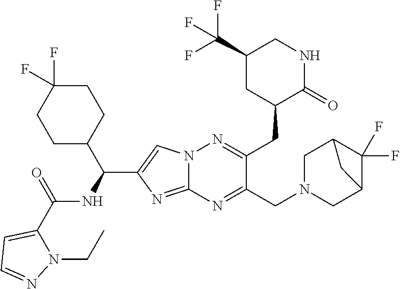 [SMILES: CCn1nccc1C(=O)N[C@H](c1cn2nc(C[C@H]3C[C@@H](C(F)(F)F)CNC3=O)c(CN3CC4CC(C3)C4(F)F)nc2n1)C1CCC(F)(F)CC1]
348 m/z = 714.3 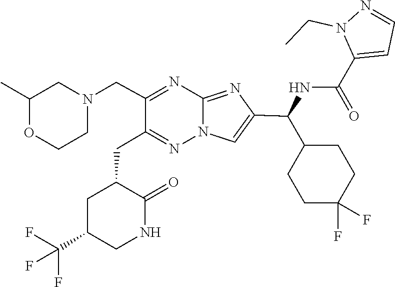 [SMILES: CCn1nccc1C(=O)N[C@H](c1cn2nc(C[C@H]3C[C@@H](C(F)(F)F)CNC3=O)c(CN3CCOC(C)C3)nc2n1)C1CCC(F)(F)CC1]
349 m/z = 682.3 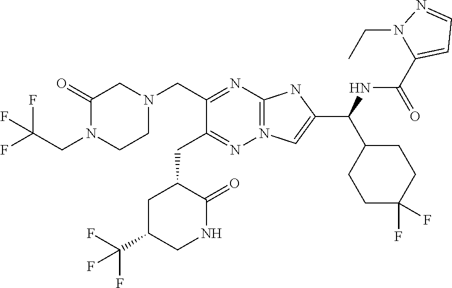
350 m/z = 682.4 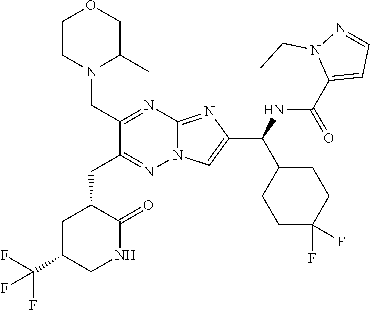 [SMILES: CCn1nccc1C(=O)N[C@H](c1cn2nc(C[C@H]3C[C@@H](C(F)(F)F)CNC3=O)c(CN3CCOCC3C)nc2n1)C1CCC(F)(F)CC1]
351 m/z = 696.4 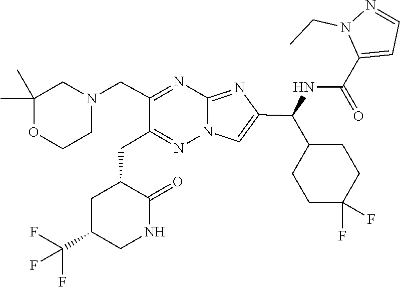 [SMILES: CCn1nccc1C(=O)N[C@H](c1cn2nc(C[C@H]3C[C@@H](C(F)(F)F)CNC3=O)c(CN3CCOC(C)(C)C3)nc2n1)C1CCC(F)(F)CC1]
352 m/z = 763.3 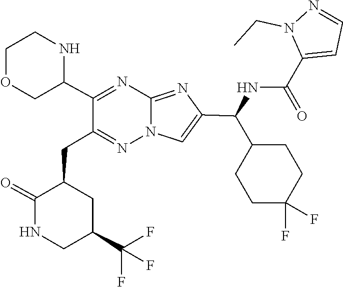 [SMILES: CCn1nccc1C(=O)N[C@H](c1cn2nc(C[C@H]3C[C@@H](C(F)(F)F)CNC3=O)c(C3COCCN3)nc2n1)C1CCC(F)(F)CC1]
353 m/z = 654.4 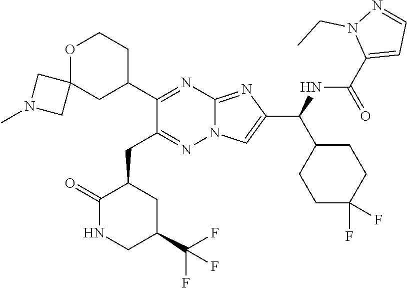 [SMILES: CCn1nccc1C(=O)N[C@H](c1cn2nc(C[C@H]3C[C@@H](C(F)(F)F)CNC3=O)c(C3CCOC4(C3)CN(C)C4)nc2n1)C1CCC(F)(F)CC1]
354 m/z = 708.4 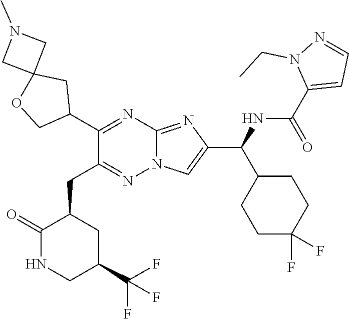 [SMILES: CCn1nccc1C(=O)N[C@H](c1cn2nc(C[C@H]3C[C@@H](C(F)(F)F)CNC3=O)c(C3COC4(C3)CN(C)C4)nc2n1)C1CCC(F)(F)CC1]
355 m/z = 694.5 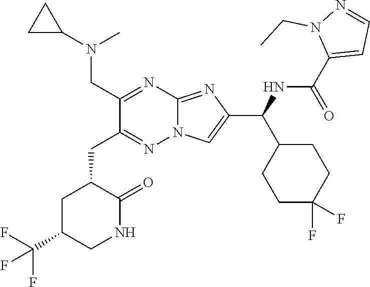 [SMILES: CCn1nccc1C(=O)N[C@H](c1cn2nc(C[C@H]3C[C@@H](C(F)(F)F)CNC3=O)c(CN(C)C3CC3)nc2n1)C1CCC(F)(F)CC1]
356 m/z = 652.4 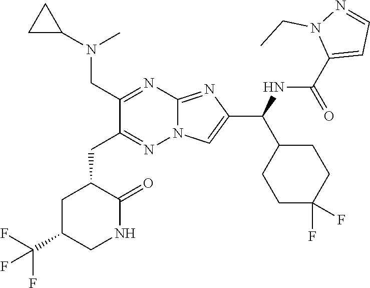 [SMILES: CCn1nccc1C(=O)N[C@H](c1cn2nc(C[C@H]3C[C@@H](C(F)(F)F)CNC3=O)c(CN(C)C3CC3)nc2n1)C1CCC(F)(F)CC1]
357 m/z = 693.6 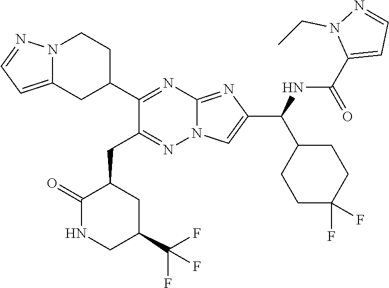 [SMILES: CCn1nccc1C(=O)N[C@H](c1cn2nc(C[C@H]3C[C@@H](C(F)(F)F)CNC3=O)c(C3CCn4nccc4C3)nc2n1)C1CCC(F)(F)CC1]
358 m/z = 689.3 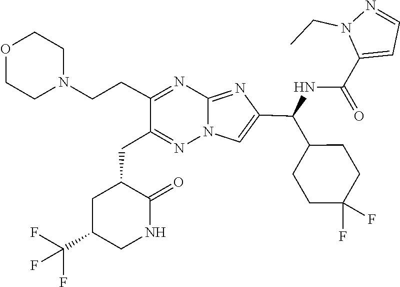 [SMILES: CCn1nccc1C(=O)N[C@H](c1cn2nc(C[C@H]3C[C@@H](C(F)(F)F)CNC3=O)c(CCN3CCOCC3)nc2n1)C1CCC(F)(F)CC1]
359 m/z = 682.3 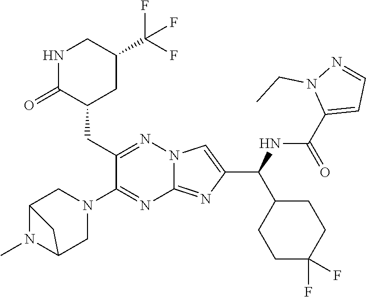 [SMILES: CCn1nccc1C(=O)N[C@H](c1cn2nc(C[C@H]3C[C@@H](C(F)(F)F)CNC3=O)c(N3CC4CC(C3)N4C)nc2n1)C1CCC(F)(F)CC1]
360 m/z = 679.3 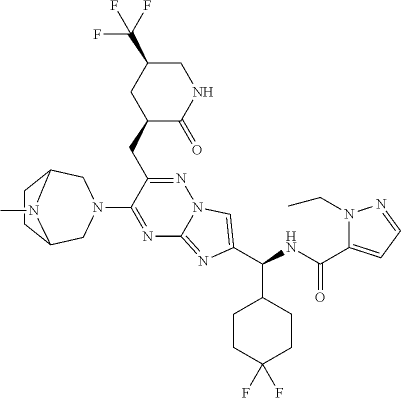 [SMILES: CCn1nccc1C(=O)N[C@H](c1cn2nc(C[C@H]3C[C@@H](C(F)(F)F)CNC3=O)c(N3CC4CCC(C3)N4C)nc2n1)C1CCC(F)(F)CC1]
361 m/z = 693.2 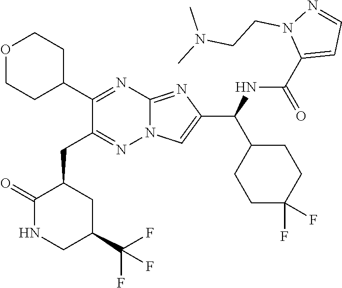 [SMILES: CN(C)CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
362 m/z = 696.3 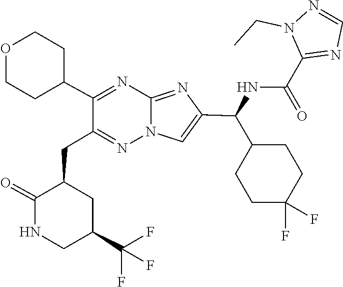 [SMILES: CCn1ncnc1C(=O)N[C@H](c1cn2nc(C[C@H]3C[C@@H](C(F)(F)F)CNC3=O)c(C3CCOCC3)nc2n1)C1CCC(F)(F)CC1]
363 m/z = 654.3 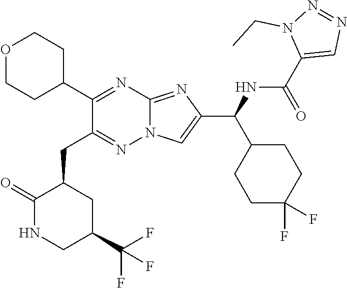 [SMILES: CCn1nncc1C(=O)N[C@H](c1cn2nc(C[C@H]3C[C@@H](C(F)(F)F)CNC3=O)c(C3CCOCC3)nc2n1)C1CCC(F)(F)CC1]
364 m/z = 654.4 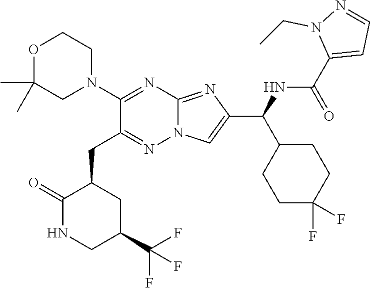 [SMILES: CCn1nccc1C(=O)N[C@H](c1cn2nc(C[C@H]3C[C@@H](C(F)(F)F)CNC3=O)c(N3CCOC(C)(C)C3)nc2n1)C1CCC(F)(F)CC1]
365 m/z = 682.3 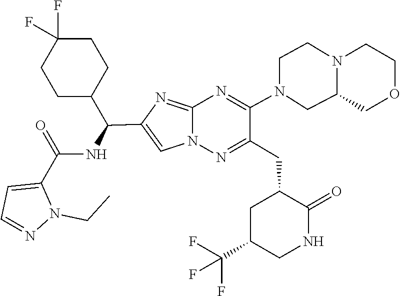 [SMILES: CCn1nccc1C(=O)N[C@H](c1cn2nc(C[C@H]3C[C@@H](C(F)(F)F)CNC3=O)c(N3CCN4CCOC[C@@H]4C3)nc2n1)C1CCC(F)(F)CC1]
366 m/z = 709.4 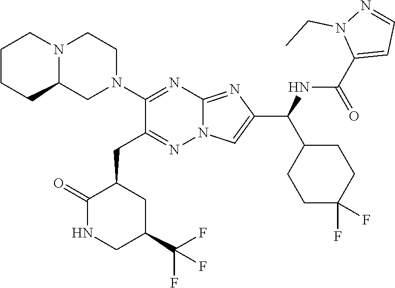 [SMILES: CCn1nccc1C(=O)N[C@H](c1cn2nc(C[C@H]3C[C@@H](C(F)(F)F)CNC3=O)c(N3CCN4CCCC[C@@H]4C3)nc2n1)C1CCC(F)(F)CC1]
367 m/z = 707.4 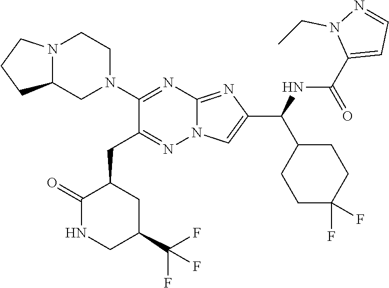 [SMILES: CCn1nccc1C(=O)N[C@H](c1cn2nc(C[C@H]3C[C@@H](C(F)(F)F)CNC3=O)c(N3CCN4CCC[C@@H]4C3)nc2n1)C1CCC(F)(F)CC1]
368 m/z = 693.4 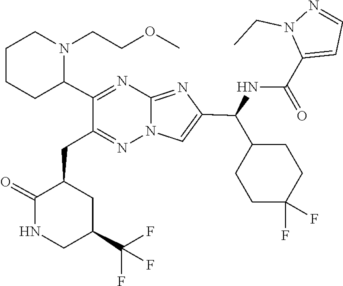 [SMILES: CCn1nccc1C(=O)N[C@H](c1cn2nc(C[C@H]3C[C@@H](C(F)(F)F)CNC3=O)c(C3CCCCN3CCOC)nc2n1)C1CCC(F)(F)CC1]
369 m/z = 710.4 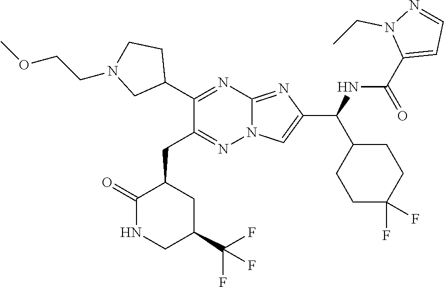 [SMILES: CCn1nccc1C(=O)N[C@H](c1cn2nc(C[C@H]3C[C@@H](C(F)(F)F)CNC3=O)c(C3CCN(CCOC)C3)nc2n1)C1CCC(F)(F)CC1]
370 m/z = 696.4 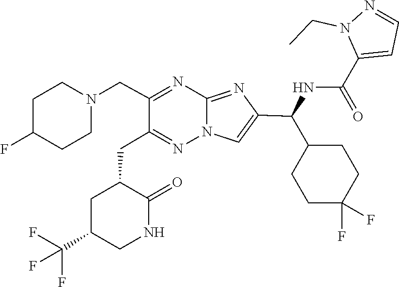 [SMILES: CCn1nccc1C(=O)N[C@H](c1cn2nc(C[C@H]3C[C@@H](C(F)(F)F)CNC3=O)c(CN3CCC(F)CC3)nc2n1)C1CCC(F)(F)CC1]
371 m/z = 684.4 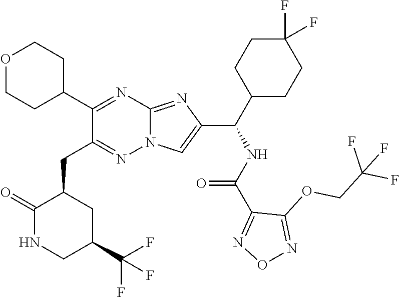 [SMILES: O=C(N[C@H](c1cn2nc(C[C@H]3C[C@@H](C(F)(F)F)CNC3=O)c(C3CCOCC3)nc2n1)C1CCC(F)(F)CC1)c1nonc1OCC(F)(F)F]
372 m/z = 725.3 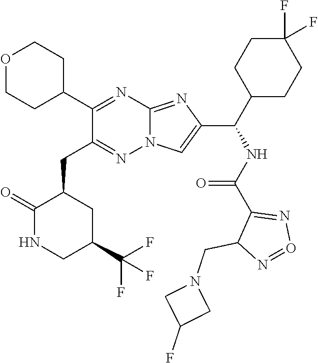
373 m/z = 714.4 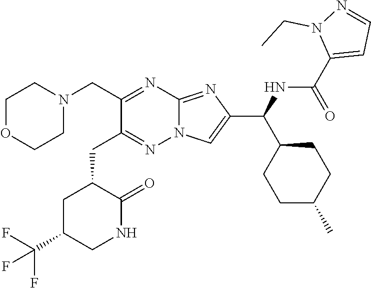 [SMILES: CCn1nccc1C(=O)N[C@H](c1cn2nc(C[C@H]3C[C@@H](C(F)(F)F)CNC3=O)c(CN3CCOCC3)nc2n1)[C@H]1CC[C@H](C)CC1]
374 m/z = 646.5 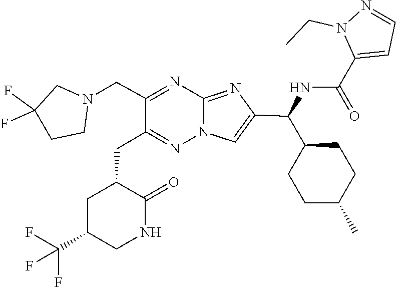 [SMILES: CCn1nccc1C(=O)N[C@H](c1cn2nc(C[C@H]3C[C@@H](C(F)(F)F)CNC3=O)c(CN3CCC(F)(F)C3)nc2n1)[C@H]1CC[C@H](C)CC1]
375 m/z = 666.4 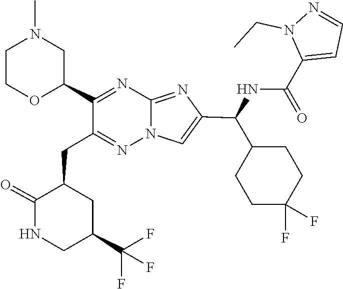 [SMILES: CCn1nccc1C(=O)N[C@H](c1cn2nc(C[C@H]3C[C@@H](C(F)(F)F)CNC3=O)c([C@@H]3CN(C)CCO3)nc2n1)C1CCC(F)(F)CC1]
376 m/z = 668.3 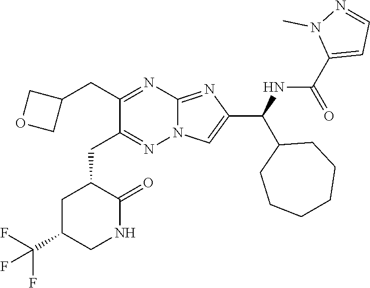 [SMILES: Cn1nccc1C(=O)N[C@H](c1cn2nc(C[C@H]3C[C@@H](C(F)(F)F)CNC3=O)c(CC3COC3)nc2n1)C1CCCCCC1]
377 m/z = 603.3 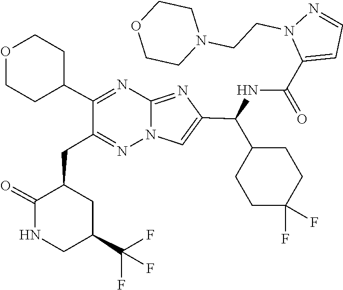 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1CCN1CCOCC1]
378 m/z = 738.5 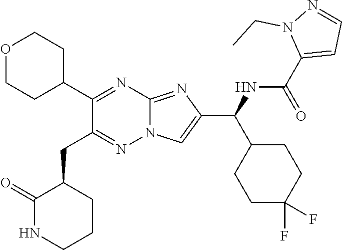 [SMILES: CCn1nccc1C(=O)N[C@H](c1cn2nc(C[C@H]3CCCNC3=O)c(C3CCOCC3)nc2n1)C1CCC(F)(F)CC1]
379 m/z = 585.4 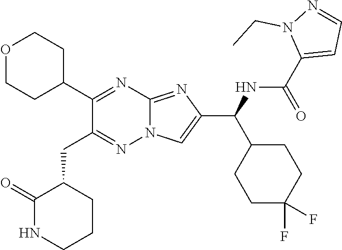 [SMILES: CCn1nccc1C(=O)N[C@H](c1cn2nc(C[C@@H]3CCCNC3=O)c(C3CCOCC3)nc2n1)C1CCC(F)(F)CC1]
380 m/z = 585.4 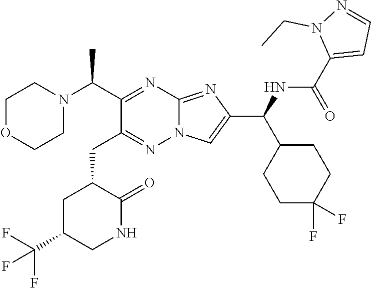 [SMILES: CCn1nccc1C(=O)N[C@H](c1cn2nc(C[C@H]3C[C@@H](C(F)(F)F)CNC3=O)c([C@H](C)N3CCOCC3)nc2n1)C1CCC(F)(F)CC1]
381 m/z = 682.4 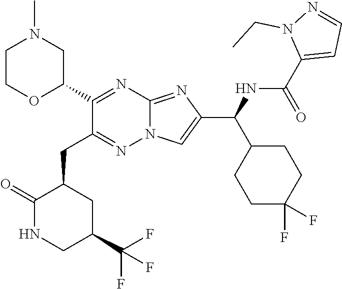 [SMILES: CCn1nccc1C(=O)N[C@H](c1cn2nc(C[C@H]3C[C@@H](C(F)(F)F)CNC3=O)c([C@H]3CN(C)CCO3)nc2n1)C1CCC(F)(F)CC1]
382 m/z = 668.3 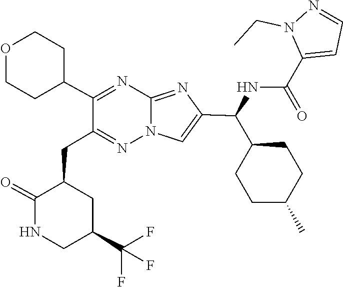 [SMILES: CCn1nccc1C(=O)N[C@H](c1cn2nc(C[C@H]3C[C@@H](C(F)(F)F)CNC3=O)c(C3CCOCC3)nc2n1)[C@H]1CC[C@H](C)CC1]
383 m/z = 632.4 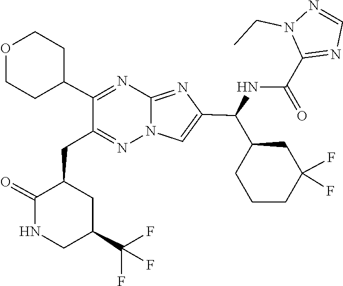 [SMILES: CCn1ncnc1C(=O)N[C@H](c1cn2nc(C[C@H]3C[C@@H](C(F)(F)F)CNC3=O)c(C3CCOCC3)nc2n1)[C@@H]1CCCC(F)(F)C1]
384 m/z = 654.3 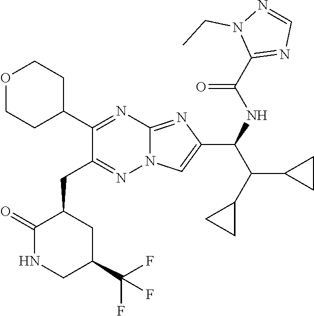 [SMILES: CCn1ncnc1C(=O)N[C@H](c1cn2nc(C[C@H]3C[C@@H](C(F)(F)F)CNC3=O)c(C3CCOCC3)nc2n1)C(C1CC1)C1CC1]
385 m/z = 630.4 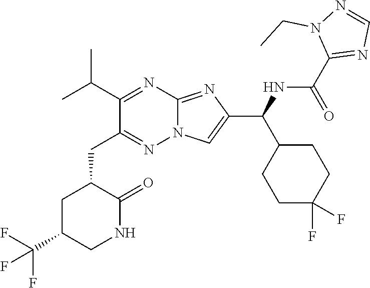 [SMILES: CCn1ncnc1C(=O)N[C@H](c1cn2nc(C[C@H]3C[C@@H](C(F)(F)F)CNC3=O)c(C(C)C)nc2n1)C1CCC(F)(F)CC1]
386 m/z = 612.3 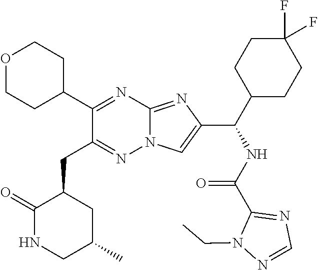 [SMILES: CCn1ncnc1C(=O)N[C@H](c1cn2nc(C[C@H]3C[C@H](C)CNC3=O)c(C3CCOCC3)nc2n1)C1CCC(F)(F)CC1]
387 m/z = 600.4 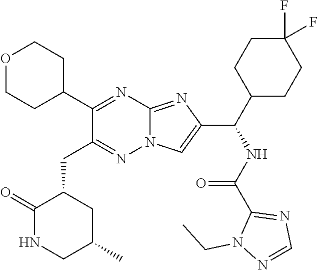 [SMILES: CCn1ncnc1C(=O)N[C@H](c1cn2nc(C[C@@H]3C[C@H](C)CNC3=O)c(C3CCOCC3)nc2n1)C1CCC(F)(F)CC1]
388 m/z = 600.4 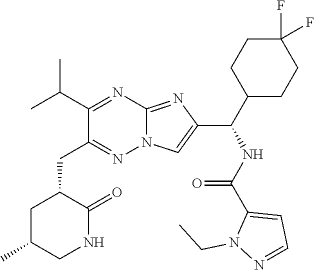 [SMILES: CCn1nccc1C(=O)N[C@H](c1cn2nc(C[C@H]3C[C@@H](C)CNC3=O)c(C(C)C)nc2n1)C1CCC(F)(F)CC1]
389 m/z = 557.3 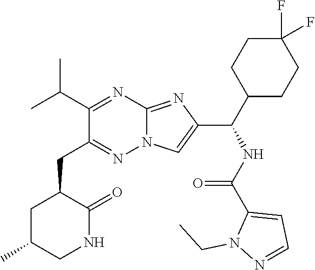 [SMILES: CCn1nccc1C(=O)N[C@H](c1cn2nc(C[C@@H]3C[C@@H](C)CNC3=O)c(C(C)C)nc2n1)C1CCC(F)(F)CC1]
390 m/z = 557.3 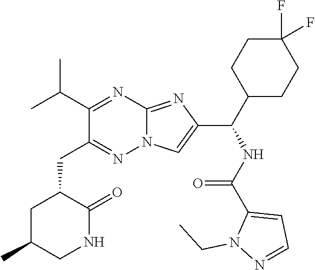 [SMILES: CCn1nccc1C(=O)N[C@H](c1cn2nc(C[C@H]3C[C@H](C)CNC3=O)c(C(C)C)nc2n1)C1CCC(F)(F)CC1]
391 m/z = 557.4 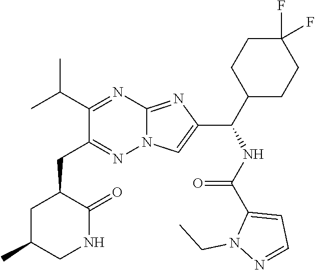 [SMILES: CCn1nccc1C(=O)N[C@H](c1cn2nc(C[C@@H]3C[C@H](C)CNC3=O)c(C(C)C)nc2n1)C1CCC(F)(F)CC1]
392 m/z = 557.4 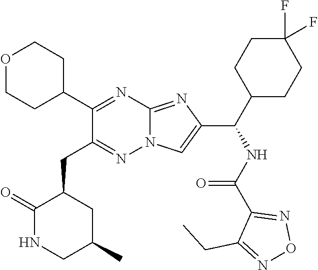 [SMILES: CCc1nonc1C(=O)N[C@H](c1cn2nc(C[C@H]3C[C@@H](C)CNC3=O)c(C3CCOCC3)nc2n1)C1CCC(F)(F)CC1]
393 m/z = 601.3 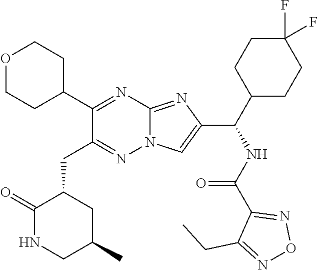 [SMILES: CCc1nonc1C(=O)N[C@H](c1cn2nc(C[C@@H]3C[C@@H](C)CNC3=O)c(C3CCOCC3)nc2n1)C1CCC(F)(F)CC1]
394 m/z = 601.3 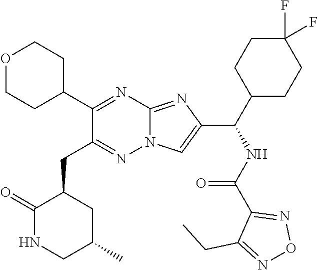 [SMILES: CCc1nonc1C(=O)N[C@H](c1cn2nc(C[C@H]3C[C@H](C)CNC3=O)c(C3CCOCC3)nc2n1)C1CCC(F)(F)CC1]
395 m/z = 601.6 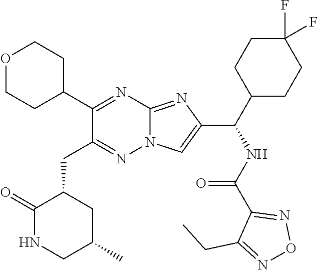 [SMILES: CCc1nonc1C(=O)N[C@H](c1cn2nc(C[C@@H]3C[C@H](C)CNC3=O)c(C3CCOCC3)nc2n1)C1CCC(F)(F)CC1]
396 m/z = 601.4 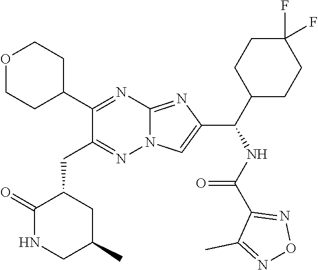 [SMILES: Cc1nonc1C(=O)N[C@H](c1cn2nc(C[C@@H]3C[C@@H](C)CNC3=O)c(C3CCOCC3)nc2n1)C1CCC(F)(F)CC1]
397 m/z = 587.4 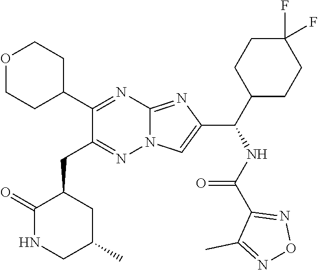 [SMILES: Cc1nonc1C(=O)N[C@H](c1cn2nc(C[C@H]3C[C@H](C)CNC3=O)c(C3CCOCC3)nc2n1)C1CCC(F)(F)CC1]
398 m/z = 587.4 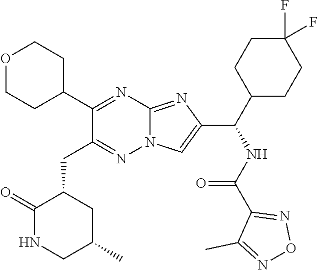 [SMILES: Cc1nonc1C(=O)N[C@H](c1cn2nc(C[C@@H]3C[C@H](C)CNC3=O)c(C3CCOCC3)nc2n1)C1CCC(F)(F)CC1]
399 m/z = 587.4 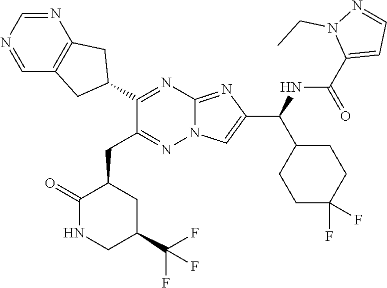 [SMILES: CCn1nccc1C(=O)N[C@H](c1cn2nc(C[C@H]3C[C@@H](C(F)(F)F)CNC3=O)c([C@@H]3Cc4cncnc4C3)nc2n1)C1CCC(F)(F)CC1]
400 m/z = 687.4 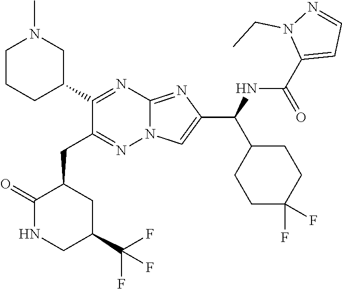 [SMILES: CCn1nccc1C(=O)N[C@H](c1cn2nc(C[C@H]3C[C@@H](C(F)(F)F)CNC3=O)c([C@@H]3CCCN(C)C3)nc2n1)C1CCC(F)(F)CC1]
401 m/z = 666.4 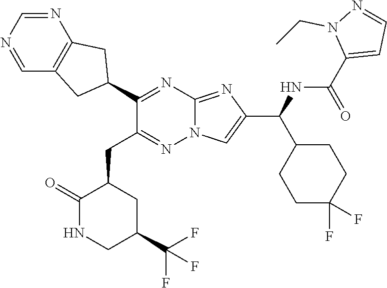 [SMILES: CCn1nccc1C(=O)N[C@H](c1cn2nc(C[C@H]3C[C@@H](C(F)(F)F)CNC3=O)c([C@H]3Cc4cncnc4C3)nc2n1)C1CCC(F)(F)CC1]
402 m/z = 687.4 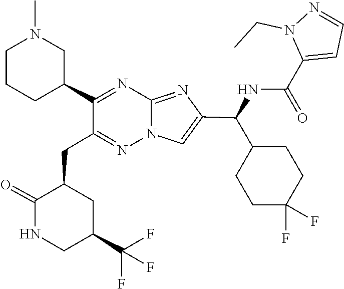 [SMILES: CCn1nccc1C(=O)N[C@H](c1cn2nc(C[C@H]3C[C@@H](C(F)(F)F)CNC3=O)c([C@H]3CCCN(C)C3)nc2n1)C1CCC(F)(F)CC1]
403 m/z = 666.4 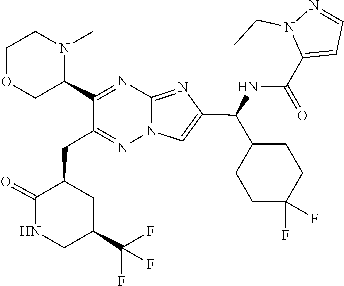 [SMILES: CCn1nccc1C(=O)N[C@H](c1cn2nc(C[C@H]3C[C@@H](C(F)(F)F)CNC3=O)c([C@H]3COCCN3C)nc2n1)C1CCC(F)(F)CC1]
404 m/z = 668.4 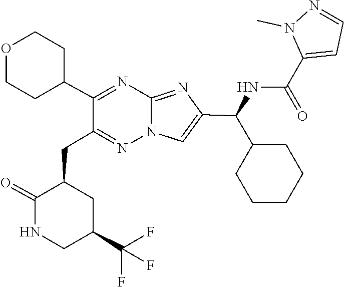 [SMILES: Cn1nccc1C(=O)N[C@H](c1cn2nc(C[C@H]3C[C@@H](C(F)(F)F)CNC3=O)c(C3CCOCC3)nc2n1)C1CCCCC1]
405 m/z = 603.4 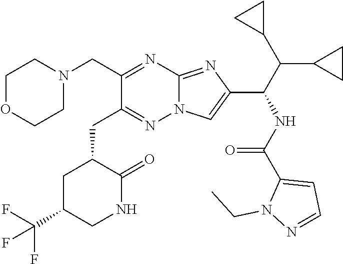 [SMILES: CCn1nccc1C(=O)N[C@H](c1cn2nc(C[C@H]3C[C@@H](C(F)(F)F)CNC3=O)c(CN3CCOCC3)nc2n1)C(C1CC1)C1CC1]
406 m/z = 644.3 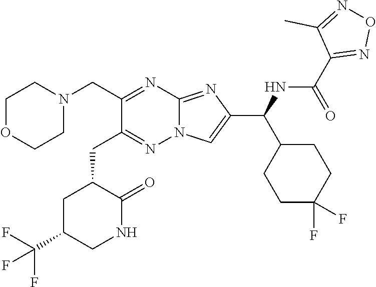 [SMILES: Cc1nonc1C(=O)N[C@H](c1cn2nc(C[C@H]3C[C@@H](C(F)(F)F)CNC3=O)c(CN3CCOCC3)nc2n1)C1CCC(F)(F)CC1]
407 m/z = 656.2 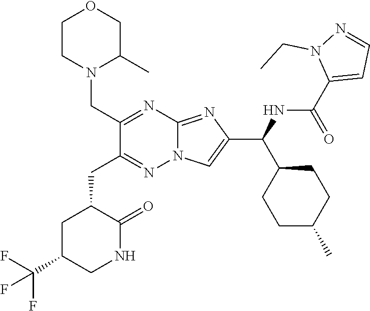 [SMILES: CCn1nccc1C(=O)N[C@H](c1cn2nc(C[C@H]3C[C@@H](C(F)(F)F)CNC3=O)c(CN3CCOCC3C)nc2n1)[C@H]1CC[C@H](C)CC1]
408 m/z = 660.5 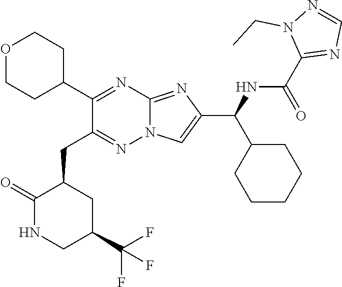 [SMILES: CCn1ncnc1C(=O)N[C@H](c1cn2nc(C[C@H]3C[C@@H](C(F)(F)F)CNC3=O)c(C3CCOCC3)nc2n1)C1CCCCC1]
409 m/z = 618.4 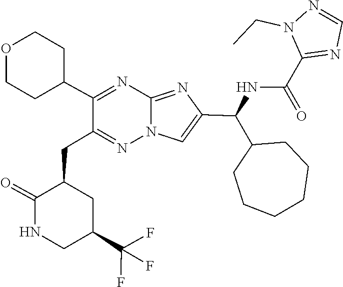 [SMILES: CCn1ncnc1C(=O)N[C@H](c1cn2nc(C[C@H]3C[C@@H](C(F)(F)F)CNC3=O)c(C3CCOCC3)nc2n1)C1CCCCCC1]
410 m/z = 632.3 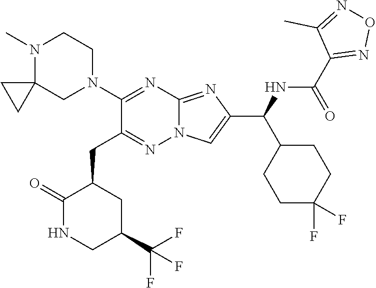 [SMILES: Cc1nonc1C(=O)N[C@H](c1cn2nc(C[C@H]3C[C@@H](C(F)(F)F)CNC3=O)c(N3CCN(C)C4(CC4)C3)nc2n1)C1CCC(F)(F)CC1]
411 m/z = 681.3 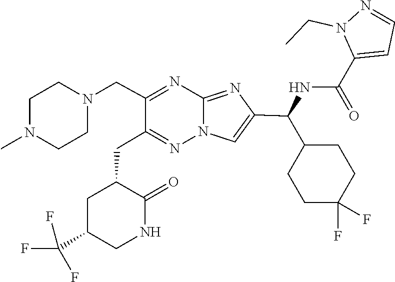 [SMILES: CCn1nccc1C(=O)N[C@H](c1cn2nc(C[C@H]3C[C@@H](C(F)(F)F)CNC3=O)c(CN3CCN(C)CC3)nc2n1)C1CCC(F)(F)CC1]
412 m/z = 681.6 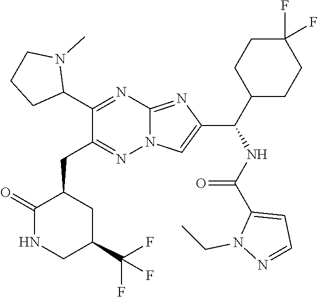 [SMILES: CCn1nccc1C(=O)N[C@H](c1cn2nc(C[C@H]3C[C@@H](C(F)(F)F)CNC3=O)c(C3CCCN3C)nc2n1)C1CCC(F)(F)CC1]
413 m/z = 652.3 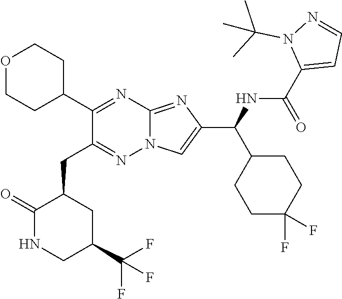 [SMILES: CC(C)(C)n1nccc1C(=O)N[C@H](c1cn2nc(C[C@H]3C[C@@H](C(F)(F)F)CNC3=O)c(C3CCOCC3)nc2n1)C1CCC(F)(F)CC1]
414 m/z = 681.4 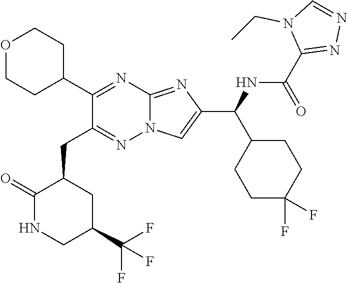 [SMILES: CCn1cnnc1C(=O)N[C@H](c1cn2nc(C[C@H]3C[C@@H](C(F)(F)F)CNC3=O)c(C3CCOCC3)nc2n1)C1CCC(F)(F)CC1]
415 m/z = 654.2 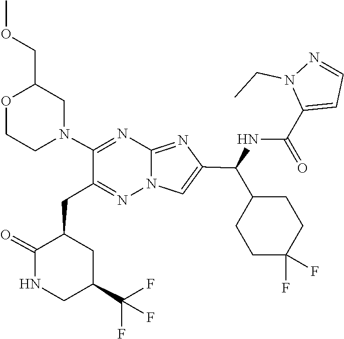 [SMILES: CCn1nccc1C(=O)N[C@H](c1cn2nc(C[C@H]3C[C@@H](C(F)(F)F)CNC3=O)c(N3CCOC(COC)C3)nc2n1)C1CCC(F)(F)CC1]
416 m/z = 698.3 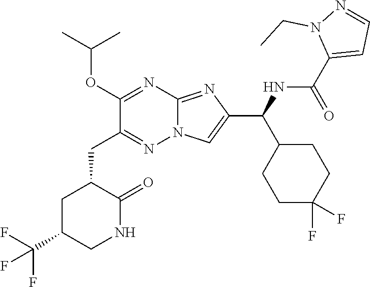 [SMILES: CCn1nccc1C(=O)N[C@H](c1cn2nc(C[C@H]3C[C@@H](C(F)(F)F)CNC3=O)c(OC(C)C)nc2n1)C1CCC(F)(F)CC1]
417 m/z = 627.3 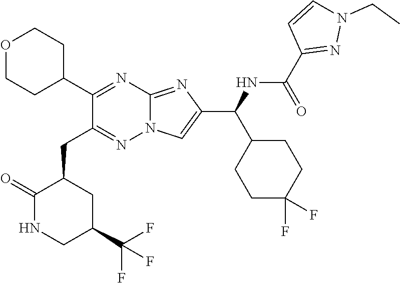 [SMILES: CCn1ccc(C(=O)N[C@H](c2cn3nc(C[C@H]4C[C@@H](C(F)(F)F)CNC4=O)c(C4CCOCC4)nc3n2)C2CCC(F)(F)CC2)n1]
418 m/z = 653.3 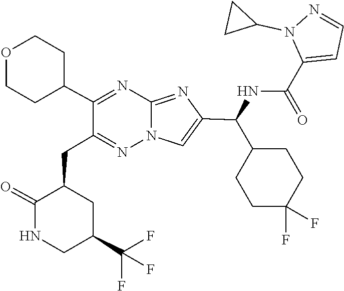 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1C1CC1]
419 m/z = 665.3 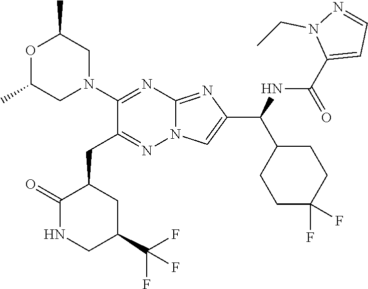 [SMILES: CCn1nccc1C(=O)N[C@H](c1cn2nc(C[C@H]3C[C@@H](C(F)(F)F)CNC3=O)c(N3C[C@H](C)O[C@@H](C)C3)nc2n1)C1CCC(F)(F)CC1]
420 m/z = 682.3 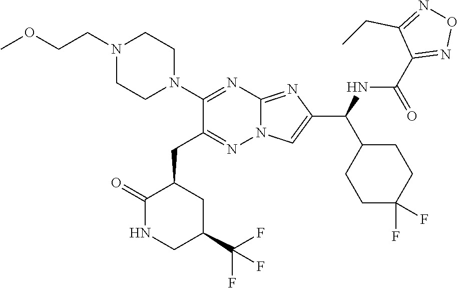 [SMILES: CCc1nonc1C(=O)N[C@H](c1cn2nc(C[C@H]3C[C@@H](C(F)(F)F)CNC3=O)c(N3CCN(CCOC)CC3)nc2n1)C1CCC(F)(F)CC1]
421 m/z = 713.5 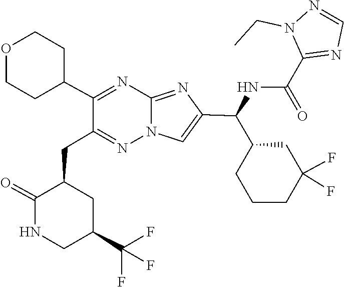 [SMILES: CCn1ncnc1C(=O)N[C@H](c1cn2nc(C[C@H]3C[C@@H](C(F)(F)F)CNC3=O)c(C3CCOCC3)nc2n1)[C@H]1CCCC(F)(F)C1]
422 m/z = 654.3 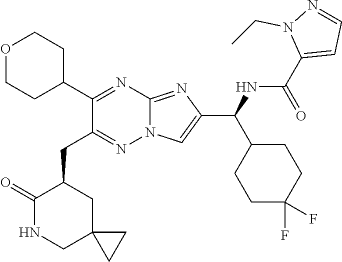 [SMILES: CCn1nccc1C(=O)N[C@H](c1cn2nc(C[C@H]3CC4(CC4)CNC3=O)c(C3CCOCC3)nc2n1)C1CCC(F)(F)CC1]
423 m/z = 611.5 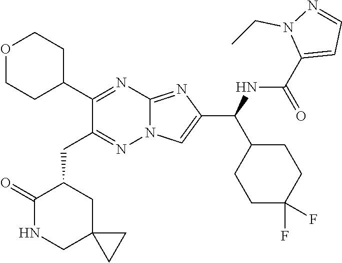 [SMILES: CCn1nccc1C(=O)N[C@H](c1cn2nc(C[C@@H]3CC4(CC4)CNC3=O)c(C3CCOCC3)nc2n1)C1CCC(F)(F)CC1]
424 m/z = 611.4 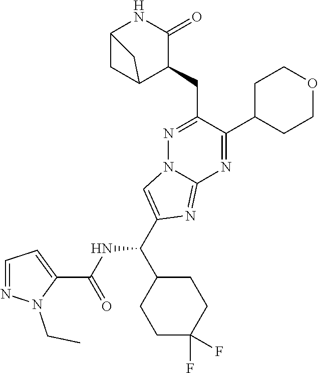 [SMILES: CCn1nccc1C(=O)N[C@H](c1cn2nc(C[C@@H]3C(=O)NC4CC3C4)c(C3CCOCC3)nc2n1)C1CCC(F)(F)CC1]
+NL425 m/z = 597.4 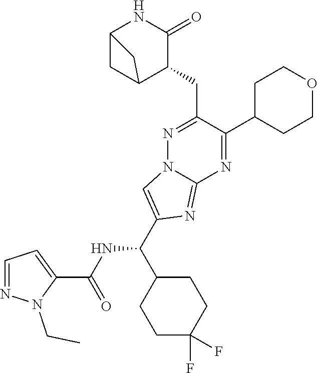 [SMILES: CCn1nccc1C(=O)N[C@H](c1cn2nc(C[C@H]3C(=O)NC4CC3C4)c(C3CCOCC3)nc2n1)C1CCC(F)(F)CC1]
426 m/z = 597.5 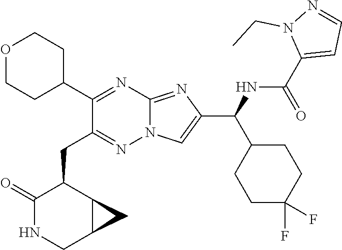 [SMILES: CCn1nccc1C(=O)N[C@H](c1cn2nc(C[C@@H]3C(=O)NC[C@@H]4C[C@@H]43)c(C3CCOCC3)nc2n1)C1CCC(F)(F)CC1]
427 m/z = 597.3 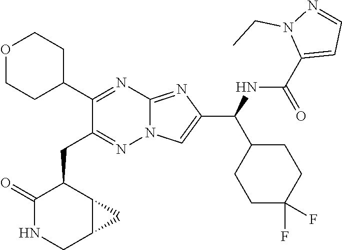 [SMILES: CCn1nccc1C(=O)N[C@H](c1cn2nc(C[C@@H]3C(=O)NC[C@H]4C[C@H]43)c(C3CCOCC3)nc2n1)C1CCC(F)(F)CC1]
428 m/z = 597.4 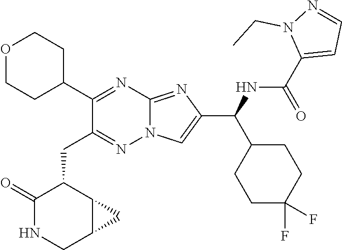 [SMILES: CCn1nccc1C(=O)N[C@H](c1cn2nc(C[C@H]3C(=O)NC[C@H]4C[C@H]43)c(C3CCOCC3)nc2n1)C1CCC(F)(F)CC1]
429 m/z = 597.3 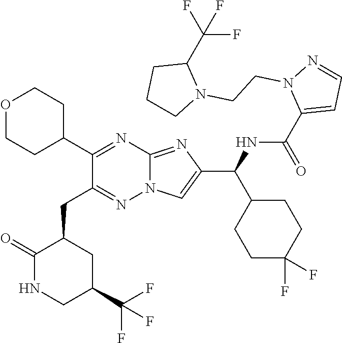 [SMILES: O=C(N[C@H](c1cn2nc(C[C@H]3C[C@@H](C(F)(F)F)CNC3=O)c(C3CCOCC3)nc2n1)C1CCC(F)(F)CC1)c1ccnn1CCN1CCCC1C(F)(F)F]
430 m/z = 790.2 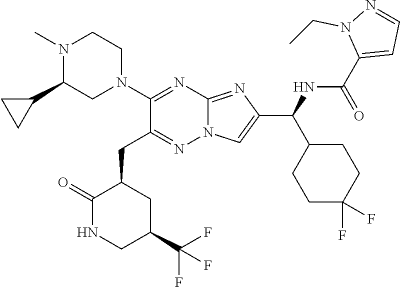 [SMILES: CCn1nccc1C(=O)N[C@H](c1cn2nc(C[C@H]3C[C@@H](C(F)(F)F)CNC3=O)c(N3CCN(C)[C@H](C4CC4)C3)nc2n1)C1CCC(F)(F)CC1]
431 m/z = 707.3 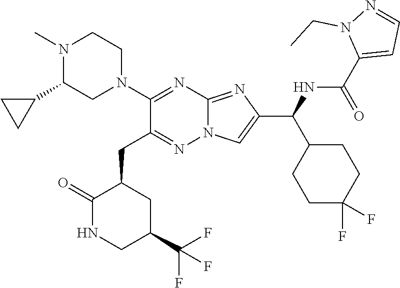 [SMILES: CCn1nccc1C(=O)N[C@H](c1cn2nc(C[C@H]3C[C@@H](C(F)(F)F)CNC3=O)c(N3CCN(C)[C@@H](C4CC4)C3)nc2n1)C1CCC(F)(F)CC1]
432 m/z = 707.3 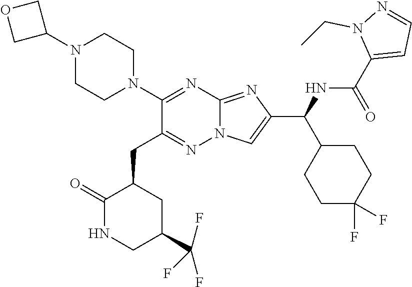 [SMILES: CCn1nccc1C(=O)N[C@H](c1cn2nc(C[C@H]3C[C@@H](C(F)(F)F)CNC3=O)c(N3CCN(C4COC4)CC3)nc2n1)C1CCC(F)(F)CC1]
433 m/z = 709.4 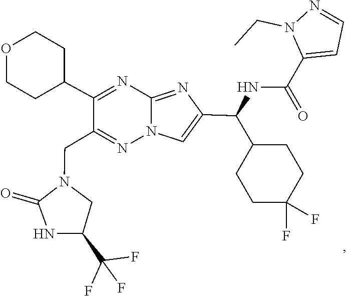 [SMILES: CCn1nccc1C(=O)N[C@H](c1cn2nc(CN3C[C@@H](C(F)(F)F)NC3=O)c(C3CCOCC3)nc2n1)C1CCC(F)(F)CC1]
434 m/z = 640.5 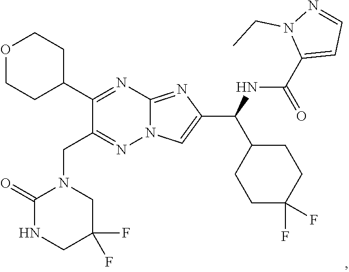 [SMILES: CCn1nccc1C(=O)N[C@H](c1cn2nc(CN3CC(F)(F)CNC3=O)c(C3CCOCC3)nc2n1)C1CCC(F)(F)CC1]
435 m/z = 622.4 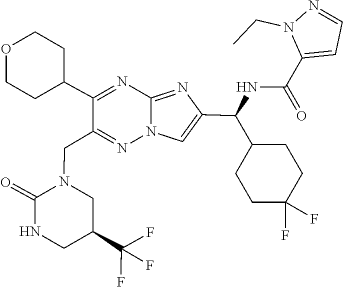 [SMILES: CCn1nccc1C(=O)N[C@H](c1cn2nc(CN3C[C@@H](C(F)(F)F)CNC3=O)c(C3CCOCC3)nc2n1)C1CCC(F)(F)CC1]
436 m/z = 654.4 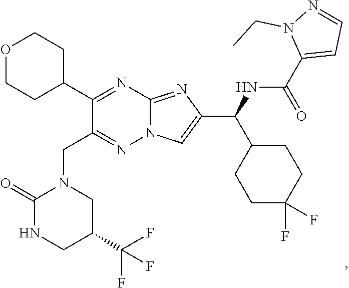 [SMILES: CCn1nccc1C(=O)N[C@H](c1cn2nc(CN3C[C@H](C(F)(F)F)CNC3=O)c(C3CCOCC3)nc2n1)C1CCC(F)(F)CC1]
437 m/z = 654.4 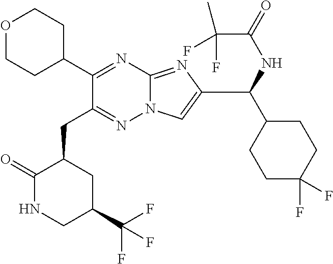 [SMILES: CC(F)(F)C(=O)N[C@H](c1cn2nc(C[C@H]3C[C@@H](C(F)(F)F)CNC3=O)c(C3CCOCC3)nc2n1)C1CCC(F)(F)CC1]
438 m/z = 623.2 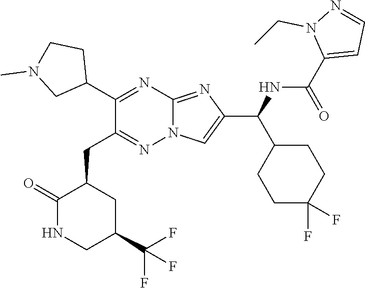 [SMILES: CCn1nccc1C(=O)N[C@H](c1cn2nc(C[C@H]3C[C@@H](C(F)(F)F)CNC3=O)c(C3CCN(C)C3)nc2n1)C1CCC(F)(F)CC1]
439 m/z = 652.4

### Example 314: IL-17A/A HEK-Blue Cell Assay

The HEK-Blue IL-17A reporter cell line (Fisher #NC 1408637) was used for cell-based IL-17A/A inhibition assays. Cells were grown and prepared for assays according to the manufacturer's instructions. This cell line consists of HEK 293 cells that were designed to expressed IL-17RA, IL-17RC, and the Act1 adapter molecule, the combination of which, when stimulated by IL-17A/A or IL17A/F activates a NFαB promoter and drives expression of the recombinant Secreted Alkaline Phosphatase (SEAP) gene. Media from the cells is then added to a development reagent (Quanti-Blue Substrate, Fisher #NC971 1613), and read at A₆₃₀.

Compounds were dispensed in DMSO to an empty clear 384-well tissue culture plate in a titration ranging from 10 μM to 27 pM, with DMSO added to every well to a final concentration of 0.100. Cells were then added to the plate (45 μL/well at a concentration of 280,000 cells/mL). IL17A/A (Genscript #Z03228) or IL17A/F (R&D Systems) was added to the plate to a final concentration of 5 ng/mL and a final well volume of 50 μL. The cells, compound, and IL-17A/A or IL17A/F were then incubated for 20 hours before media was removed for SEAP analysis. The resulting inhibition curve was then analyzed using Dotmatics' integrated 4-parameter fit screening protocol to generate IC₅₀ values.

Table 2 includes IC₅₀ values for IL-17A/A and IL-17A/F inhibition of selected compounds; with compounds having a IC₅₀ of A<100 nM; B 100-1000 nM; and C>1000 nM.

TABLE 2
IL-17 A/A and IL-17 A/F Inhibition Data
for selected compounds

| | HEK- | HEK- |
|---|---|---|
| Compound | Blue | Blue |
| No. | IL17AA | IL17AF |
| 1 | A | C |
| 2 | B | C |
| 3 | B | C |
| 4 | A | C |
| 5 | B | C |
| 6 | A | C |
| 7 | B | C |
| 8 | B | C |
| 9 | A | C |
| 10 | B | C |
| 11 | B | C |
| 12 | A | C |
| 13 | A | C |
| 14 | B | |
| 15 | B | C |
| 16 | B | C |
| 17 | A | B |
| 18 | A | C |
| 19 | B | C |
| 20 | A | C |
| 21 | A | C |
| 22 | A | B |
| 23 | A | C |
| 24 | B | C |
| 25 | B | C |
| 26 | A | C |
| 27 | A | C |
| 28 | B | C |
| 29 | A | C |
| 30 | A | C |
| 31 | B | C |
| 32 | B | C |
| 33 | B | C |
| 34 | A | C |
| 35 | B | C |
| 36 | B | C |
| 37 | A | C |
| 38 | B | C |
| 39 | A | C |
| 40 | B | C |
| 41 | B | C |
| 42 | B | C |
| 43 | A | C |
| 44 | A | C |
| 45 | B | C |
| 46 | A | B |
| 47 | A | C |
| 48 | A | B |
| 49 | A | C |
| 50 | B | C |
| 51 | B | C |
| 52 | B | |
| 53 | B | C |
| 54 | A | B |
| 55 | A | B |
| 56 | A | C |
| 57 | A | C |
| 58 | A | B |
| 59 | A | C |
| 60 | A | C |
| 61 | A | B |
| 62 | A | B |
| 63 | B | C |
| 64 | A | C |
| 65 | A | C |
| 66 | A | A |
| 67 | A | A |
| 68 | A | A |
| 69 | A | B |
| 70 | A | A |
| 71 | A | B |
| 72 | A | B |
| 73 | A | B |
| 74 | A | B |
| 75 | A | B |
| 76 | B | C |
| 77 | A | C |
| 78 | A | A |
| 79 | A | B |
| 80 | A | C |
| 81 | A | B |
| 82 | A | C |
| 83 | A | B |
| 84 | A | B |
| 85 | B | C |
| 86 | A | B |
| 87 | A | B |
| 88 | A | B |
| 89 | A | C |
| 90 | A | B |
| 91 | A | B |
| 92 | A | B |
| 93 | A | B |
| 94 | A | B |
| 95 | A | B |
| 96 | A | B |
| 97 | A | C |
| 98 | A | A |
| 99 | A | C |
| 100 | A | A |
| 101 | A | B |
| 102 | A | A |
| 103 | A | B |
| 104 | A | A |
| 105 | A | B |
| 106 | A | C |
| 107 | A | B |
| 108 | A | A |
| 109 | A | B |
| 110 | A | B |
| 111 | A | B |
| 112 | A | B |
| 113 | A | B |
| 114 | A | C |
| 115 | A | C |
| 116 | A | C |
| 117 | A | B |
| 118 | B | C |
| 119 | A | B |
| 120 | B | C |
| 121 | A | A |
| 122 | A | A |
| 123 | A | B |
| 124 | A | B |
| 125 | A | C |
| 126 | A | B |
| 127 | A | B |
| 128 | A | B |
| 129 | A | B |
| 130 | A | C |
| 131 | A | C |
| 132 | A | B |
| 133 | A | B |
| 134 | A | C |
| 135 | A | C |
| 136 | A | C |
| 137 | A | B |
| 138 | A | A |
| 139 | A | A |
| 140 | A | B |
| 141 | A | A |
| 142 | A | C |
| 143 | A | B |
| 144 | A | B |
| 145 | A | B |
| 146 | A | B |
| 147 | A | B |
| 148 | A | C |
| 149 | A | B |
| 150 | A | B |
| 151 | A | B |
| 152 | A | B |
| 153 | A | B |
| 154 | A | C |
| 155 | A | B |
| 156 | A | C |
| 157 | A | A |
| 158 | A | B |
| 159 | A | C |
| 160 | A | B |
| 161 | A | C |
| 162 | A | B |
| 163 | A | B |
| 164 | A | B |
| 165 | A | B |
| 166 | A | B |
| 167 | A | B |
| 168 | A | B |
| 169 | A | C |
| 170 | A | B |
| 171 | A | B |
| 172 | A | B |
| 173 | B | C |
| 174 | A | B |
| 175 | A | B |
| 176 | A | A |
| 177 | A | B |
| 178 | A | C |
| 179 | A | A |
| 180 | B | C |
| 181 | A | C |
| 182 | A | A |
| 183 | A | B |
| 184 | A | C |
| 185 | A | B |
| 186 | A | B |
| 187 | A | B |
| 188 | A | B |
| 189 | A | B |
| 190 | A | C |
| 191 | A | B |
| 192 | A | B |
| 193 | A | B |
| 194 | A | B |
| 195 | B | C |
| 196 | A | A |
| 197 | A | C |
| 198 | A | B |
| 200 | A | B |
| 201 | A | B |
| 202 | B | |
| 203 | A | A |
| 204 | A | B |
| 205 | A | A |
| 206 | A | B |
| 207 | A | B |
| 208 | A | B |
| 209 | A | C |
| 210 | A | B |
| 211 | A | A |
| 212 | A | B |
| 213 | A | B |
| 214 | A | B |
| 215 | A | B |
| 216 | A | B |
| 217 | A | C |
| 218 | A | A |
| 219 | A | B |
| 220 | A | B |
| 221 | A | B |
| 222 | A | C |
| 223 | A | B |
| 224 | A | B |
| 225 | A | B |
| 226 | A | C |
| 227 | A | B |
| 228 | A | C |
| 229 | A | C |
| 230 | B | C |
| 231 | A | B |
| 232 | A | B |
| 233 | A | B |
| 234 | A | B |
| 235 | B | C |
| 236 | A | B |
| 237 | A | B |
| 238 | B | B |
| 239 | B | C |
| 240 | B | B |
| 241 | A | A |
| 242 | A | B |
| 243 | A | C |
| 244 | A | B |
| 245 | A | B |
| 246 | B | C |
| 247 | A | B |
| 248 | A | C |
| 249 | A | B |
| 250 | A | B |
| 251 | A | C |
| 252 | A | B |
| 253 | A | B |
| 254 | A | B |
| 255 | A | B |
| 256 | A | B |
| 257 | A | B |
| 258 | A | B |
| 259 | A | A |
| 260 | A | B |
| 261 | A | B |
| 262 | A | B |
| 263 | A | C |
| 264 | A | B |
| 265 | A | C |
| 266 | A | C |
| 267 | A | B |
| 268 | A | B |
| 269 | A | C |
| 270 | A | C |
| 271 | A | B |
| 272 | A | A |
| 273 | A | C |
| 274 | A | C |
| 275 | A | C |
| 276 | A | B |
| 277 | A | B |
| 278 | A | B |
| 279 | A | C |
| 280 | A | C |
| 281 | A | B |
| 282 | A | A |
| 283 | A | B |
| 284 | B | C |
| 285 | B | C |
| 286 | B | C |
| 287 | B | C |
| 288 | A | C |
| 289 | A | A |
| 290 | A | A |
| 291 | A | C |
| 292 | A | C |
| 293 | A | A |
| 294 | A | A |
| 295 | A | C |
| 296 | A | B |
| 297 | A | B |
| 298 | A | A |
| 299 | B | C |
| 300 | B | C |
| 301 | A | C |
| 302 | A | C |
| 303 | A | C |
| 304 | A | C |
| 305 | A | A |
| 306 | A | B |
| 307 | A | A |
| 308 | A | B |
| 309 | A | B |
| 310 | A | B |
| 311 | A | C |
| 312 | A | C |
| 313 | A | C |
| 314 | A | C |
| 315 | A | C |
| 316 | B | C |
| 317 | B | C |
| 318 | A | C |
| 319 | A | C |
| 320 | A | B |
| 321 | A | B |
| 322 | A | B |
| 323 | A | C |
| 324 | B | C |
| 325 | B | C |
| 326 | A | B |
| 327 | A | C |
| 328 | A | B |
| 329 | A | B |
| 330 | A | B |
| 331 | A | B |
| 332 | A | C |
| 333 | A | C |
| 334 | A | B |
| 335 | A | B |
| 336 | A | B |
| 337 | A | C |
| 338 | A | C |
| 339 | B | C |
| 340 | A | C |
| 341 | A | C |
| 342 | B | C |
| 343 | A | C |
| 344 | B | C |
| 345 | A | C |
| 346 | A | C |
| 347 | A | C |
| 348 | A | B |
| 349 | A | C |
| 350 | A | B |
| 351 | A | C |
| 352 | B | C |
| 353 | A | C |
| 354 | B | C |
| 355 | B | C |
| 356 | A | B |
| 357 | A | B |
| 358 | A | B |
| 359 | A | B |
| 360 | B | B |
| 361 | A | B |
| 362 | B | C |
| 363 | A | C |
| 364 | A | C |
| 365 | A | B |
| 366 | A | A |
| 367 | A | A |
| 368 | A | B |
| 369 | B | C |
| 370 | B | C |
| 371 | A | B |
| 372 | A | B |
| 373 | A | B |
| 374 | A | B |
| 375 | A | B |
| 376 | A | C |
| 377 | A | B |
| 378 | A | C |
| 379 | A | C |
| 380 | A | C |
| 381 | B | C |
| 382 | A | C |
| 383 | A | B |
| 384 | A | C |
| 385 | A | C |
| 386 | A | C |
| 387 | B | C |
| 388 | A | C |
| 389 | B | C |
| 390 | A | B |
| 391 | A | C |
| 392 | A | C |
| 393 | B | C |
| 394 | A | C |
| 395 | A | C |
| 396 | A | C |
| 397 | A | C |
| 398 | B | C |
| 399 | A | C |
| 400 | A | B |
| 401 | B | C |
| 402 | A | B |
| 403 | A | C |
| 404 | B | C |
| 405 | A | B |
| 406 | A | B |
| 407 | B | C |
| 408 | A | A |
| 409 | A | C |
| 410 | A | B |
| 411 | A | C |
| 412 | B | C |
| 413 | B | C |
| 414 | A | C |
| 415 | B | C |
| 416 | A | B |
| 417 | A | C |
| 418 | A | C |
| 419 | A | B |
| 420 | A | B |
| 421 | A | C |
| 422 | A | B |
| 423 | A | C |
| 424 | A | B |
| 425 | B | C |
| 426 | A | C |
| 427 | B | C |
| 428 | A | C |
| 429 | B | C |
| 430 | A | B |
| 431 | A | B |
| 432 | A | B |
| 433 | A | B |
| 434 | B | C |
| 435 | B | C |
| 436 | B | C |
| 437 | B | C |
| 438 | B | |
| 439 | B | C |

The data provided in Table 2 demonstrate that the compounds identified in Table 2 inhibit IL-17A/A and/or IL-17 A/F mediated signaling in the HEK-Blue Cell Assay as described in Example 314.

### Example 315: Mouse Oral Bioavailability

The test compound is administered to C57BL/6J mice intravenously (IV) at 1 mg/kg or orally (PO) at 10 mg/kg using vehicles composed of 10% DMA, 70% Polyethylene glycol 400, and 20% purified water; or 10% DMA, 50% Polyethylene glycol 400, and 40% purified water. Serial blood collections occur at 0.08, 0.25, 0.5, 1, 2, 4, 8 and 24 h post IV bolus dose administration, and 0.25, 0.5, 1, 2, 4, 8 and 24 h post oral dose administration. Blood samples are treated with EDTA anticoagulant. Plasma is isolated by centrifugation and stored at −70° C. until analysis by LC-MS/MS. Test article concentration is determined in plasma and uploaded into the Phoenix WinNonlin system where noncompartmental analysis is used to calculate Area Under the Curve (AUC) for both IV and PO arms. Bioavailability (% F) is calculated via the following equation,

% F=(AUC_{PO}×Dose_{IV})/(AUC_{IV}×Dose_{PO})×100

Table 3 indicates the mouse oral bioavailability of selected compounds.

TABLE 3
Mouse Oral Bioavailability (% F) at 10 mg/kg
of selected compounds

| Compound No. | % F (Mouse) |
|---|---|
| 56 | 87% |
| 69 | 40% |
| 70 | 45% |
| 204 | 27% |
| 75 | 58% |
| 88 | 69% |
| 122 | 51% |
| 148 | 91% |
| 153 | 43% |
| 229 | 83% |

The data provided in Table 3 illustrate the pharmacologically advantageous oral bioavailability of the compounds of the invention.

What is claimed is:

1. A compound represented by the Formula: 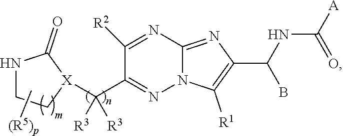
or a pharmaceutically acceptable salt thereof wherein:
X is selected from CH or N;
A is selected from): 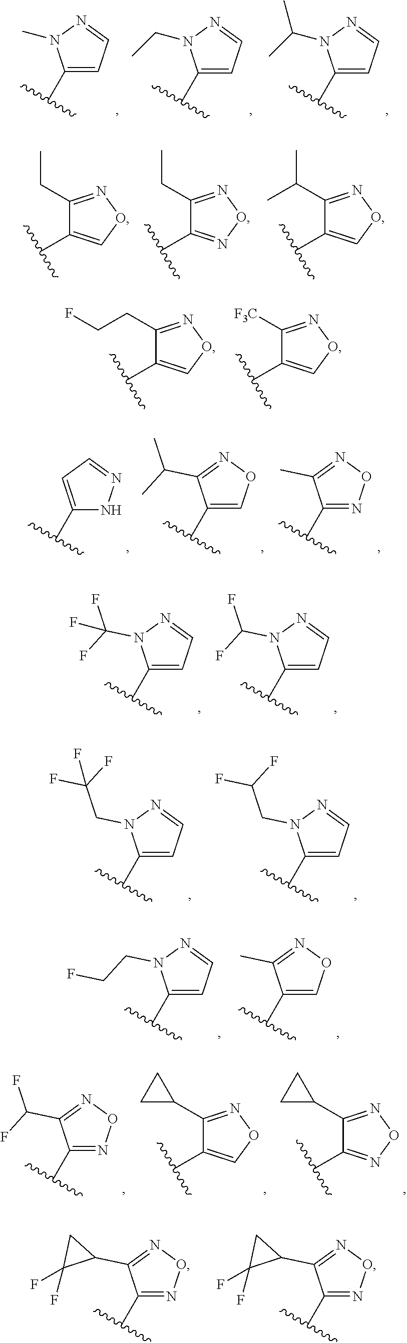 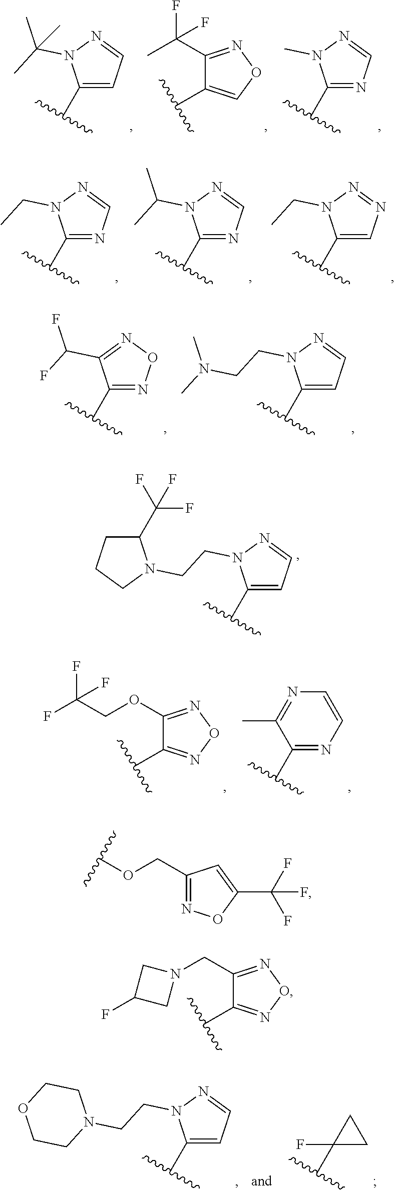
B is selected from 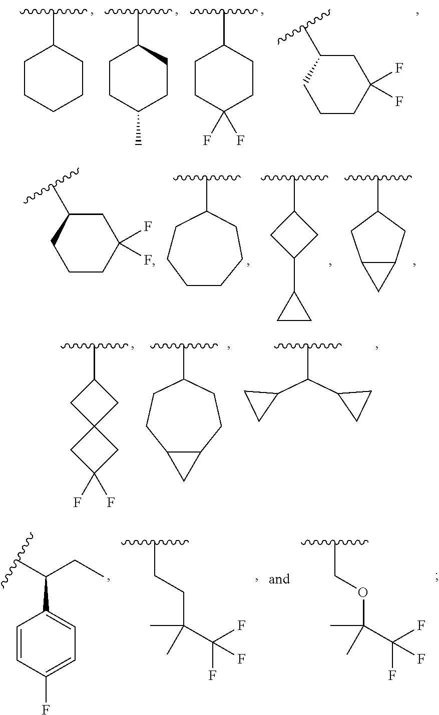
R¹ is
hydrogen;
R² is selected from:
hydrogen, 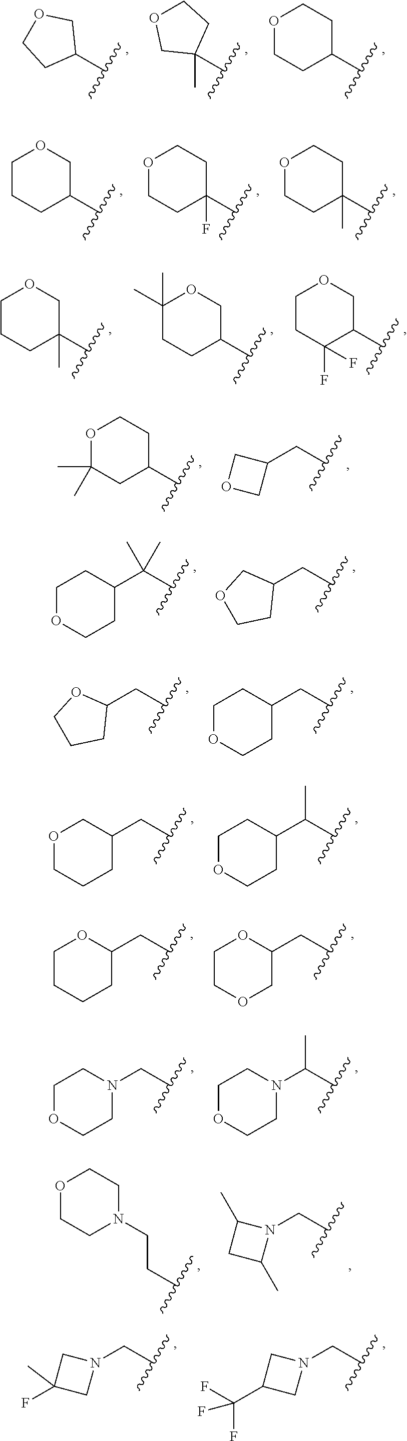 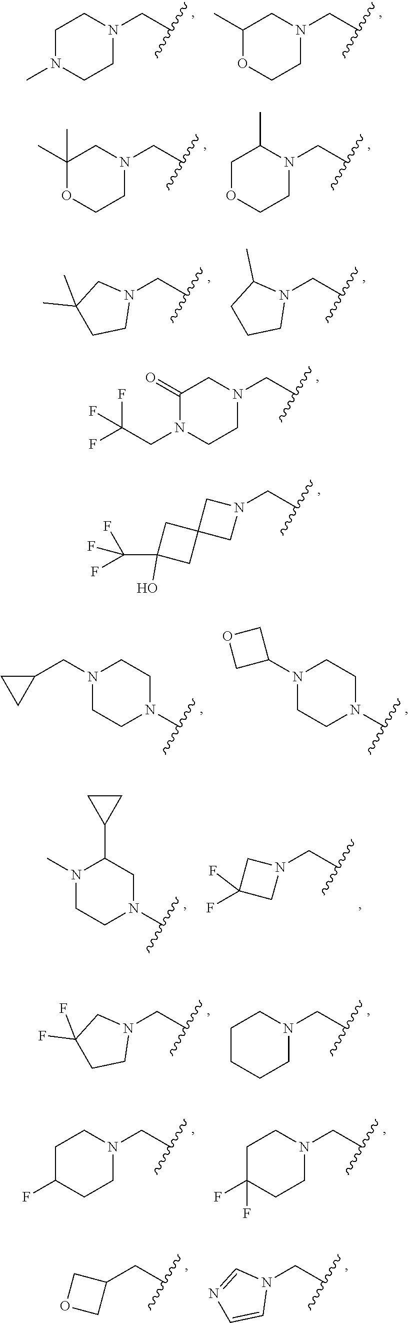 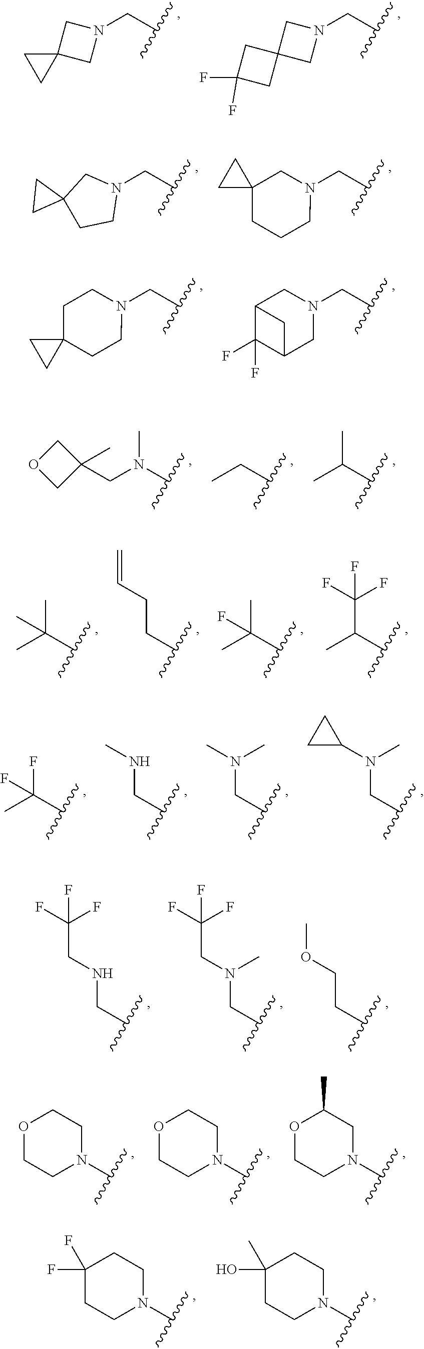 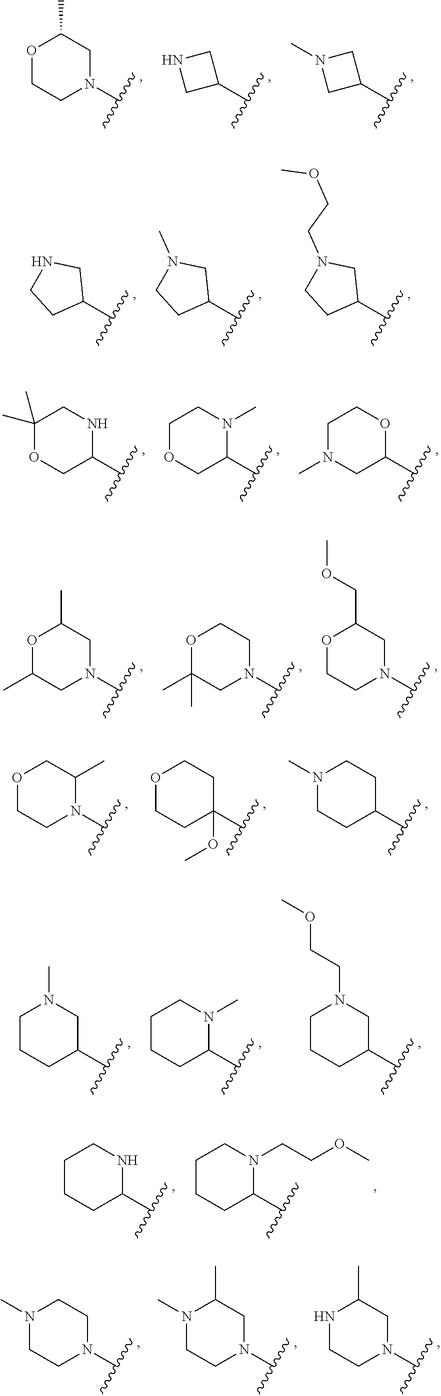 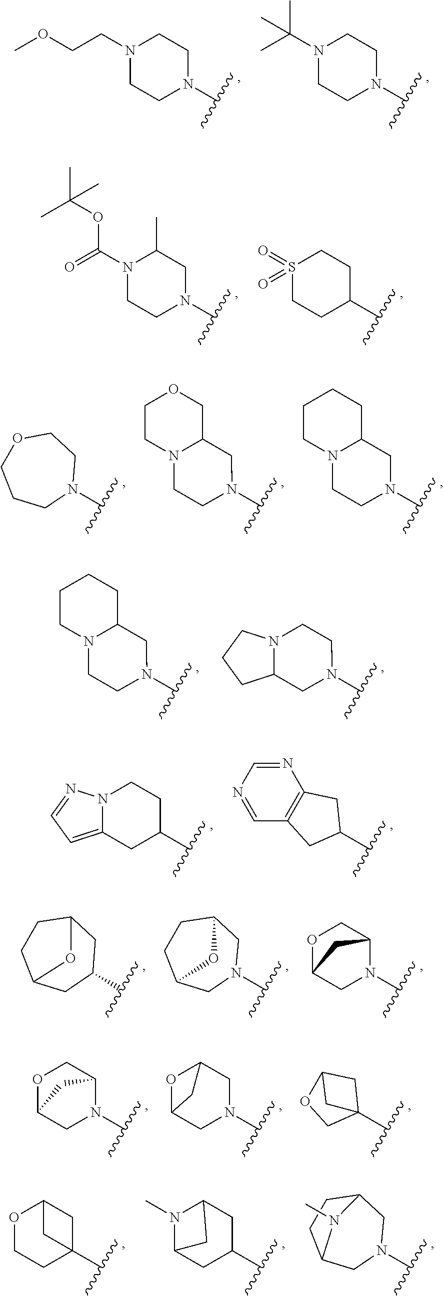 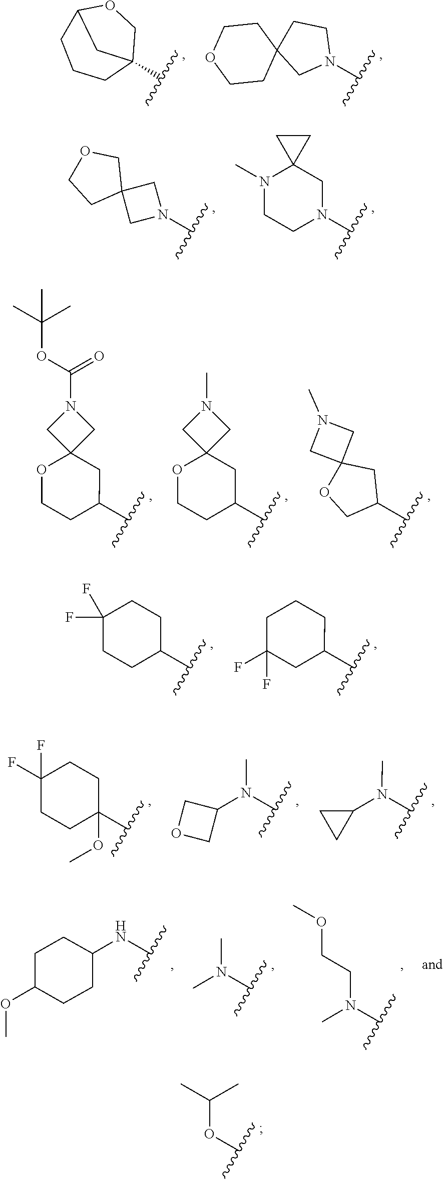
n is 1;
each R³ is hydrogen; and 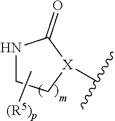
is selected from:

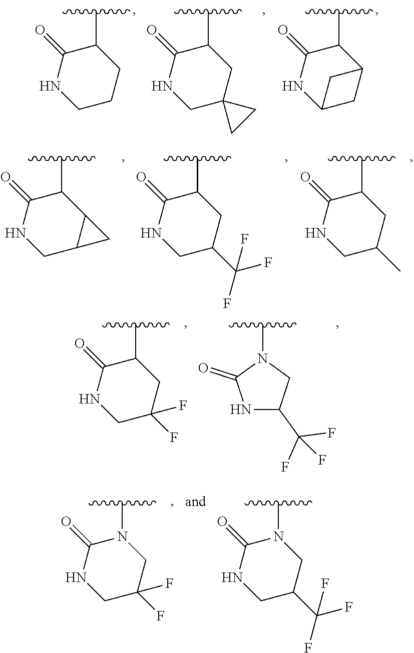

2.-147. (canceled)

148. The compound or salt of claim 1, wherein the compound
is selected from: 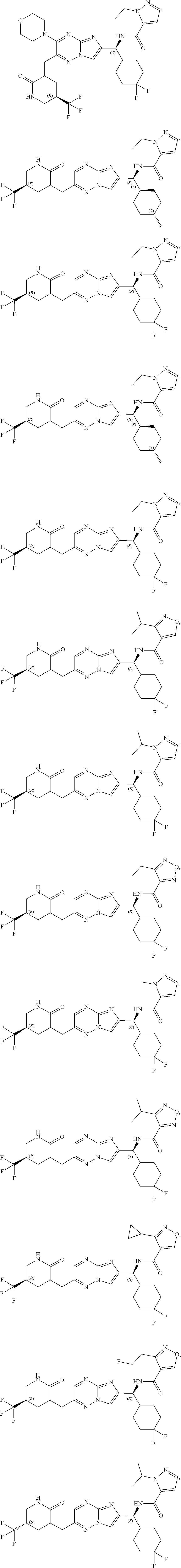 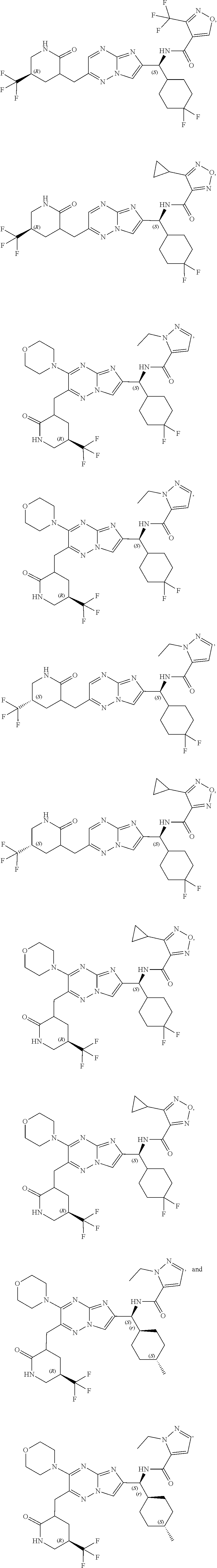
or pharmaceutically acceptable salts thereof.

149. The compound or salt of claim 1, wherein the compound is selected from the group consisting of structure 1-439 described in Table 1, or pharmaceutically acceptable salts thereof.

150. The compound or salt of claim 1, of the formula: 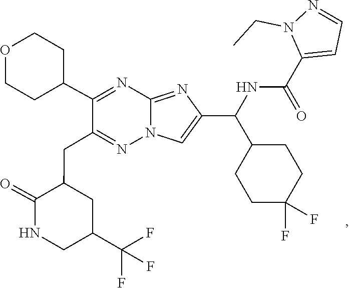 [SMILES: CCn1nccc1C(=O)NC(c1cn2nc(CC3CC(C(F)(F)F)CNC3=O)c(C3CCOCC3)nc2n1)C1CCC(F)(F)CC1]
or a pharmaceutically acceptable salt thereof.

151. The compound or salt of claim 150 of the formula: 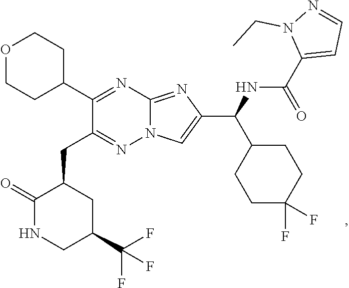 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

152. The compound of claim 151 of formula:

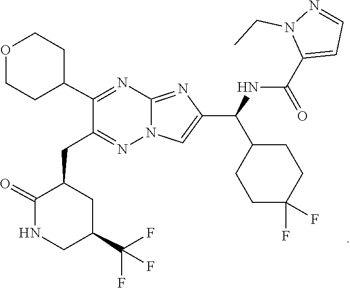 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]

153. The compound or salt of claim 150 of the formula: 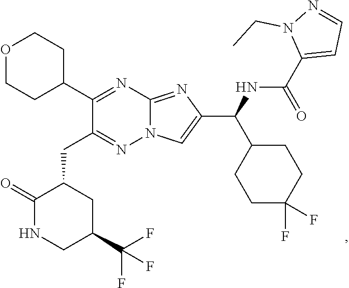 [SMILES: CCn1nccc1C(=O)N[C@H](c1cn2nc(C[C@@H]3C[C@@H](C(F)(F)F)CNC3=O)c(C3CCOCC3)nc2n1)C1CCC(F)(F)CC1]
or a pharmaceutically acceptable salt thereof.

154. The compound or salt of claim 150 of the formula: 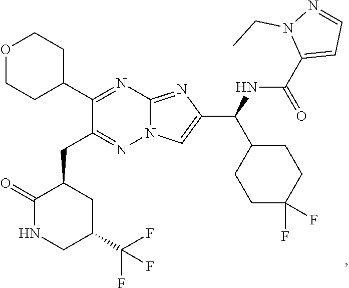 [SMILES: CCn1nccc1C(=O)N[C@H](c1cn2nc(C[C@H]3C[C@H](C(F)(F)F)CNC3=O)c(C3CCOCC3)nc2n1)C1CCC(F)(F)CC1]
or a pharmaceutically acceptable salt thereof.

155. The compound or salt of claim 1, of the formula: 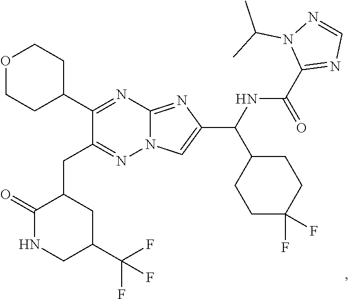 [SMILES: CC(C)n1ncnc1C(=O)NC(c1cn2nc(CC3CC(C(F)(F)F)CNC3=O)c(C3CCOCC3)nc2n1)C1CCC(F)(F)CC1]
or a pharmaceutically acceptable salt thereof.

156. The compound or salt of claim 155 of the formula: 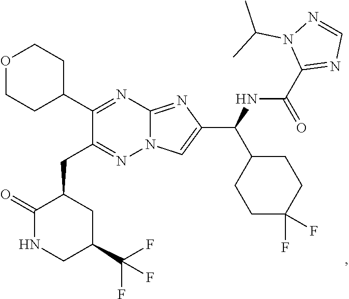 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

157. The compound of claim 156 of the formula:

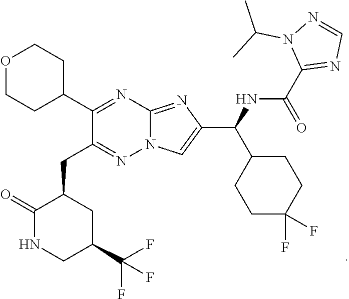 [SMILES: CC(C)n1ncnc1C(=O)N[C@H](c1cn2nc(C[C@H]3C[C@@H](C(F)(F)F)CNC3=O)c(C3CCOCC3)nc2n1)C1CCC(F)(F)CC1]

158. The compound or salt of claim 1, of the formula: 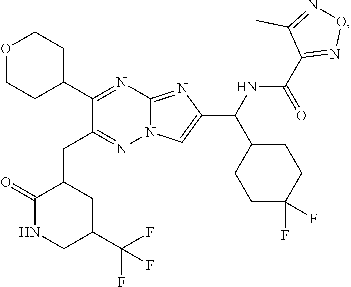 [SMILES: Cc1nonc1C(=O)NC(c1cn2nc(CC3CC(C(F)(F)F)CNC3=O)c(C3CCOCC3)nc2n1)C1CCC(F)(F)CC1]
or a pharmaceutically acceptable salt thereof.

159. The compound or salt of claim 158 of formula: 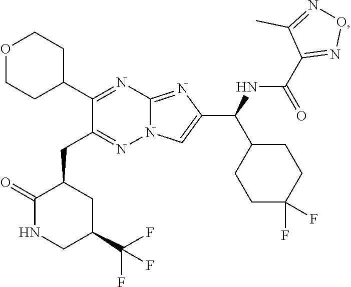 [SMILES: Cc1nonc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

160. A pharmaceutical composition comprising a compound or salt of claim 1 and a pharmaceutically acceptable excipient.

161. (canceled)

162. A method of treating an inflammatory disease or condition comprising administering to a subject in need thereof a compound or salt of claim 1.

163. The method of claim 162, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

164. The method of claim 163, wherein the method comprises administering to a subject in need thereof a compound of the formula: 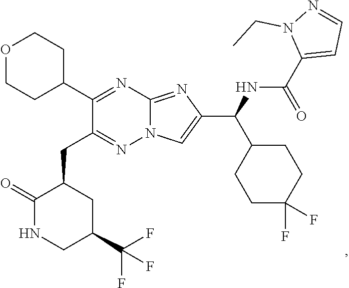 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

165. (canceled)

166. The method of claim 162 wherein the inflammatory disease or condition is psoriasis, further comprising administering to a subject in need thereof a compound of the formula: 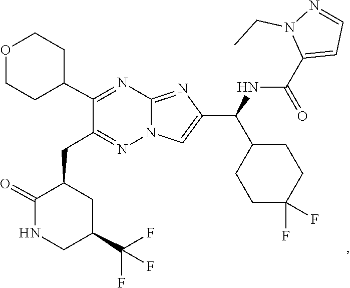 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

167. (canceled)

168. The method of claim 162 wherein the inflammatory disease or condition is psoriatic arthritis, further comprising administering to a subject in need thereof a compound of the formula: 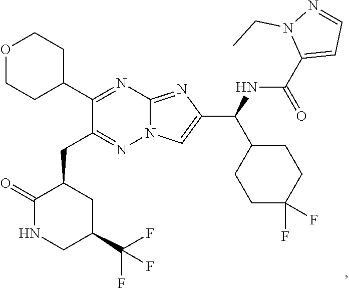 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

169. (canceled)

170. The method of claim 162 wherein the inflammatory disease or condition is ankylosing spondylitis, further comprising administering to a subject in need thereof a compound of the formula: 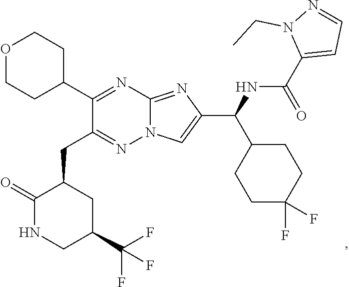 [SMILES: CCn1nccc1C(=O)N[C@H](c1cn2nc(C[C@H]3C[C@@H](C(F)(F)F)CNC3=O)c(C3CCOCC3)nc2n1)C1CCC(F)(F)CC1]
or a pharmaceutically acceptable salt thereof.

171. (canceled)
